(19) United States
(12) Patent Application Publication
(10) Pub. No.: US 2007/0293530 A1
(43) Pub. Date: Dec. 20, 2007

(54) SULFAMIDE AND SULFAMATE DERIVATIVES AS HISTONE DEACETYLASE INHIBITORS

(75) Inventors: David Smil, Montreal (CA); Silvana Leit, Kirkland (CA); Alain Ajamian, Montreal (CA); Martin Allan, Montreal (CA); Yves Andre Chantigny, Pincourt (CA); Robert Deziel, Mount-Royal (CA); Eric Therrien, Laval (CA); Amal Wahhab, Laval (CA); Sukhdev Manku, L'lle Perrot (CA)

Correspondence Address: MCDONNELL BOEHNEN HULBERT & BERGHOFF LLP, 300 S. WACKER DRIVE, 32ND FLOOR, CHICAGO, IL, 60606, US

(73) Assignee: METHYLGENE INC., Montreal (CA)

(21) Appl. No.: 11/762,874

(22) Filed: Jun. 14, 2007

Related U.S. Application Data
(60) Provisional application No. 60/804,719, filed on Jun. 14, 2006.

(51) Int. Cl. C07D 471/02; A61K 31/4745

(52) U.S. Cl. 514/292; 546/82

(57) ABSTRACT

This invention relates to compounds for the inhibition of histone deacetylase. More particularly, the invention provides for compounds of formula (I), and racemic and scalemic mixtures, diastereomers and enantiomers thereof: 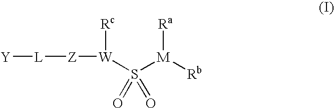
or an N-oxide, hydrate, solvate, pharmaceutically acceptable salt, prodrug or complex thereof, wherein Y, L, Z, W, M, R^{a}, R^{b} and R^{c} are as defined in the specification.

### BACKGROUND OF THE INVENTION

1. Field of the Invention

This invention relates to compounds for the inhibition of histone deacetylase.

2. Description of Related Art

In eukaryotic cells, nuclear DNA associates with histones to form a compact complex called chromatin. The histones constitute a family of basic proteins which are generally highly conserved across eukaryotic species. The core histones, termed H2A, H₂B, H3, and H4, associate to form a protein core. DNA winds around this protein core, with the basic amino acids of the histones interacting with the negatively charged phosphate groups of the DNA. Approximately 146 base pairs of DNA wrap around a histone core to make up a nucleosome particle, the repeating structural motif of chromatin.

Csordas, Biochem. J., 286: 23-38 (1990) teaches that histones are subject to posttranslational acetylation of the α,ε-amino groups of N-terminal lysine residues, a reaction that is catalyzed by histone acetyl transferase (HAT1). Acetylation neutralizes the positive charge of the lysine side chain, and is thought to impact chromatin structure. Indeed, Taunton et al., Science, 272: 408-411 (1996), teaches that access of transcription factors to chromatin templates is enhanced by histone hyperacetylation. Taunton et al. further teaches that an enrichment in underacetylated histone H4 has been found in transcriptionally silent regions of the genome.

Histone acetylation is a reversible modification, with deacetylation being catalyzed by a family of enzymes termed histone deacetylases (HDACs). The molecular cloning of gene sequences encoding proteins with HDAC activity has established the existence of a set of discrete HDAC enzyme isoforms. Grozinger et al., Proc. Natl. Acad. Sci. USA, 96:4868-4873 (1999), teaches that HDACs may be divided into two classes, the first represented by yeast Rpd3-like proteins, and the second represented by yeast Hd1-like proteins. Grozinger et al. also teaches that the human HDAC-1, HDAC-2, and HDAC-3 proteins are members of the first class of HDACs, and discloses new proteins, named HDAC-4, HDAC-5, and HDAC-6, which are members of the second class of HDACs. Kao et al., Gene & Development 14:55-66 (2000), discloses an additional member of this second class, called HDAC-7. More recently, Hu, E. et al. J. Bio. Chem. 275:15254-13264 (2000) disclosed another member of the first class of histone deacetylases, HDAC-8. Zhou et al., Proc. Natl. Acad. Sci. U.S.A., 98: 10572-10577 (2001) teaches the cloning and characterization of a new histone deacetylase, HDAC-9. Kao et al., J. Biol. Chem., 277:187-93 (2002) teaches the isolation and characterization of mammalian HDAC10, a novel histone deacetylase. Gao et al, J. Biol. Chem. 77(28):25748-55 (2002) teaches the cloning and functional characterization of HDAC11, a novel member of the human histone deacetylase family. Shore, Proc. Natl. Acad. Sci. U.S.A. 97: 14030-2 (2000) discloses a third class of deacetylase activity, the Sir2 protein family. It has been unclear what roles these individual HDAC enzymes play.

Studies utilizing known HDAC inhibitors have established a link between acetylation and gene expression. Numerous studies have examined the relationship between HDAC and gene expression. Taunton et al., Science 272:408-411 (1996), discloses a human HDAC that is related to a yeast transcriptional regulator. Cress et al., J. Cell. Phys. 184:1-16 (2000), discloses that, in the context of human cancer, the role of HDAC is as a corepressor of transcription. Ng et al., TIBS 25: March (2000), discloses HDAC as a pervasive feature of transcriptional repressor systems. Magnaghi-Jaulin et al., Prog. Cell Cycle Res. 4:41-47 (2000), discloses HDAC as a transcriptional co-regulator important for cell cycle progression.

Richon et al., Proc. Natl. Acad. Sci. USA, 95: 3003-3007 (1998), discloses that HDAC activity is inhibited by trichostatin A (TSA), a natural product isolated from Streptomyces hygroscopicus, which has been shown to inhibit histone deacetylase activity and arrest cell cycle progression in cells in the G1 and G2 phases (Yoshida et al., J. Biol. Chem. 265: 17174-17179, 1990; Yoshida et al, Exp. Cell Res. 177: 122-131, 1988), and by a synthetic compound, suberoylanilide hydroxamic acid (SAHA). Yoshida and Beppu, Exper. Cell Res., 177: 122-131 (1988), teaches that TSA causes arrest of rat fibroblasts at the G₁ and G₂ phases of the cell cycle, implicating HDAC in cell cycle regulation. Indeed, Finnin et al., Nature, 401: 188-193 (1999), teaches that TSA and SAHA inhibit cell growth, induce terminal differentiation, and prevent the formation of tumors in mice. Suzuki et al, U.S. Pat. No. 6,174,905 and EP 0847992, disclose benzamide derivatives that induce cell differentiation and inhibit HDAC. Delorme et al., WO 01/38322 and PCT/IB01/00683, disclose additional compounds that serve as HDAC inhibitors. Other inhibitors of histone deacetylase activity, including trapoxin, depudecin, FR901228 (Fujisawa Pharmaceuticals), and butyrate, have been found to similarly inhibit cell cycle progression in cells (Taunton et al., Science 272: 408-411, 1996; Kijima et al, J. Biol. Chem. 268(30):22429-22435, 1993; Kwon et al, Proc. Natl. Acad. Sci. USA 95(7):3356-61, 1998).

It would be highly desirable to have additional inhibiters of histone deacetylase.

### SUMMARY OF THE INVENTION

The present invention provides compounds for the inhibition of histone deacetylase.

In a first aspect, the present invention provides compounds that are useful as inhibitors of histone deacetylase that have the formula (I), and racemic and scalemic mixtures, diastereomers and enantiomers thereof: 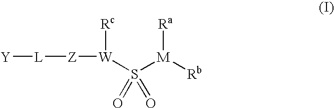
and N-oxides, hydrates, solvates, pharmaceutically acceptable salts, prodrugs and complexes thereof, wherein Y, L, Z, W, M, R^{a}, R^{b} and R^{c} are as defined below. In this first aspect, the invention provides compounds of formula I that are useful as HDAC inhibitors and, therefore, are useful research tools for the study of the role of histone deacetylases in both normal and disease states.

In a second aspect, the invention provides a composition comprising a compound according to the present invention. In a preferred embodiment, the composition further comprises an additional inhibitory agent.

In a third aspect, the invention provides a method of inhibiting histone deacetylase, the method comprising contacting the histone deacetylase or a cell containing histone deacetylase activity, with a histone deacetylase inhibiting amount of a compound according to the first aspect or a composition according to second aspect.

The foregoing merely summarizes the above aspects of the invention and is not intended to be limiting in nature. These aspects and other aspects and embodiments are described more fully below. The patent and scientific literature referred to herein establishes knowledge that is available to those with skill in the art. The issued patents, applications, and references that are cited herein are hereby incorporated by reference to the same extent as if each was specifically and individually indicated to be incorporated by reference. In the case of inconsistencies, the present disclosure will prevail.

### DETAILED DESCRIPTION OF THE INVENTION

The present invention provides compounds that are useful as inhibitors of histone deacetylase.

In one aspect, the invention provides compounds of the formula (I), and racemic and scalemic mixtures, diastereomers and enantiomers thereof: 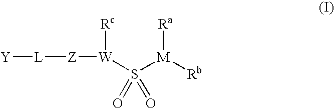
and N-oxides, hydrates, solvates, pharmaceutically acceptable salts, prodrugs and complexes thereof, wherein Y, L, Z, W, M, R^{a}, R^{b} and R^{c} are as defined herein.

In the second aspect, the invention provides a composition comprising a compound according to the first aspect or a preferred embodiment thereof and a pharmaceutically acceptable carrier.

In a third aspect, the invention provides a method of inhibiting histone deacetylase, the method comprising contacting the histone deacetylase or a cell containing histone deacetylase activity with an inhibition effective amount of a compound according to the present invention, or with an inhibition effective amount of a composition according to the present invention. Inhibition of histone deacetylase activity can be in a cell or a multicellular organism. If in a multicellular organism, the method according to this aspect of the invention comprises administering to the organism an inhibition effective amount of a compound according to the present invention, or an inhibition effective amount of a composition according to the present invention. Preferably the organism is a mammal, more preferably a human. In a preferred embodiment, the method further comprises concurrently or sequentially contacting the histone deacetylase, or the cell, with an effective amount of an additional HDAC inhibitory agent, or if in a multicellular organism, concurrently or sequentially administering an inhibition effective amount of an additional HDAC inhibitory agent.

For purposes of the present invention, the following definitions will be used (unless expressly stated otherwise).

As used herein, the terms “histone deacetylase” and “HDAC” are intended to refer to any one of a family of enzymes that remove acetyl groups from amino groups of proteins, including but not limited to amino groups of lysine residues at the N-terminus of a histone. Unless otherwise indicated by context, the term “histone” is meant to refer to any histone protein, including H1, H2A, H₂B, H3, H4, and H5, from any species. Preferred histone deacetylases include class I and class II enzymes. Other preferred histone deacetylases include class III enzymes. Preferably the histone deacetylase is a human HDAC, including, but not limited to, HDAC-1, HDAC-2, HDAC-3, HDAC-4, HDAC-5, HDAC-6, HDAC-7, HDAC-8, HDAC-9, HDAC-10 and HDAC-11. In some other preferred embodiments, the histone deacetylase is derived from a protozoal or fungal source.

The terms “histone deacetylase inhibitor” and “inhibitor of histone deacetylase” are intended to mean a compound having a structure as defined herein, which is capable of interacting with a histone deacetylase and inhibiting its enzymatic activity.

The term “inhibiting histone deacetylase enzymatic activity” is intended to mean reducing the ability of a histone deacetylase to remove an acetyl group from a protein, including but not limited to a histone. The concentration of inhibitor which reduces the activity of a histone deacetylase to 50% of that of the uninhibited enzyme is determined as the IC₅₀ value.

Preferably, such inhibition is specific, i.e., the histone deacetylase inhibitor reduces the ability of a histone deacetylase to remove an acetyl group from a protein, including but not limited to a histone, at a concentration that is lower than the concentration of the inhibitor that is required to produce another, unrelated biological effect. Preferably, the concentration of the inhibitor required for histone deacetylase inhibitory activity is at least 2-fold lower, more preferably at least 5-fold lower, even more preferably at least 10-fold lower, and most preferably at least 20-fold lower than the concentration required to produce an unrelated biological effect.

Reference to “a compound of the formula (I), formula (II), etc.,” (or equivalently, “a compound according to the first aspect”, or “a compound of the present invention”, and the like), herein is understood to include reference to N-oxides, hydrates, solvates, pharmaceutically acceptable salts, prodrugs and complexes thereof, and racemic and scalemic mixtures, diastereomers, enantiomers and tautomers thereof and unless otherwise indicated.

For simplicity, chemical moieties are defined and referred to throughout primarily as univalent chemical moieties (e.g., alkyl, aryl, etc.). Nevertheless, such terms are also used to convey corresponding multivalent moieties under the appropriate structural circumstances clear to those skilled in the art. For example, while an “alkyl” moiety generally refers to a monovalent radical (e.g. CH₃—CH₂—), in certain circumstances a bivalent linking moiety can be “alkyl,” in which case those skilled in the art will understand the alkyl to be a divalent radical (e.g., —CH₂—CH₂—), which is equivalent to the term “alkylene.” (Similarly, in circumstances in which a divalent moiety is required and is stated as being “aryl,” those skilled in the art will understand that the term “aryl” refers to the corresponding divalent moiety, arylene). All atoms are understood to have their normal number of valences for bond formation (i.e., 4 for carbon, 3 for N, 2 for 0, and 2, 4, or 6 for S, depending on the oxidation state of the S). On occasion a moiety may be defined, for example, as (A)ₐ-B-, wherein a is 0 or 1. In such instances, when a is 0 the moiety is B- and when a is 1 the moiety is A-B-. Also, a number of moietes disclosed here may exist in multiple tautomeric forms, all of which are intended to be encompassed by any given tautomeric structure.

For simplicity, reference to a “Cₙ-Cₘ” heterocyclyl or “Cₙ-Cₘ” heteroaryl means a heterocyclyl or heteroaryl having from “n” to “m” annular atoms, where “n” and “m” are integers. Thus, for example, a C₅-C₆-heterocyclyl is a 5- or 6-membered ring having at least one heteroatom, and includes pyrrolidinyl (C₅) and piperidinyl (C₆); C₆-hetoaryl includes, for example, pyridyl and pyrimidyl.

The term “hydrocarbyl” refers to a straight, branched, or cyclic alkyl, alkenyl, or alkynyl, each as defined herein. A “C₀” hydrocarbyl is used to refer to a covalent bond. Thus, “C₀-C₃-hydrocarbyl” includes a covalent bond, methyl, ethyl, ethenyl, ethynyl, propyl, propenyl, propynyl, and cyclopropyl.

The term “aliphatic” is intended to mean both saturated and unsaturated, straight chain or branched aliphatic hydrocarbons. As will be appreciated by one of ordinary skill in the art, “aliphatic” is intended herein to include, but is not limited to, alkyl, alkenyl or alkynyl moieties.

The term “alkyl” is intended to mean a straight chain or branched aliphatic group having from 1 to 12 carbon atoms, preferably 1-8 carbon atoms, and more preferably 1-6 carbon atoms. Other preferred alkyl groups have from 2 to 12 carbon atoms, preferably 2-8 carbon atoms and more preferably 2-6 carbon atoms. Preferred alkyl groups include, without limitation, methyl, ethyl, propyl, isopropyl, butyl, isobutyl, sec-butyl, tert-butyl, pentyl, hexyl and the like. A “C₀” alkyl (as in “C₀-C₃alkyl”) is a covalent bond.

The term “alkenyl” is intended to mean an unsaturated straight chain or branched aliphatic group with one or more carbon-carbon double bonds, having from 2 to 12 carbon atoms, preferably 2-8 carbon atoms, and more preferably 2-6 carbon atoms. Preferred alkenyl groups include, without limitation, ethenyl, propenyl, butenyl, pentenyl, and hexenyl.

The term “alkynyl” is intended to mean an unsaturated straight chain or branched aliphatic group with one or more carbon-carbon triple bonds, having from 2 to 12 carbon atoms, preferably 2-8 carbon atoms, and more preferably 2-6 carbon atoms. Preferred alkynyl groups include, without limitation, ethynyl, propynyl, butynyl, pentynyl, and hexynyl.

The terms “alkylene,” “alkenylene,” or “alkynylene” as used herein are intended to mean an alkyl, alkenyl, or alkynyl group, respectively, as defined hereinabove, that is positioned between and serves to connect two other chemical groups. Preferred alkylene groups include, without limitation, methylene, ethylene, propylene, and butylene. Preferred alkenylene groups include, without limitation, ethenylene, propenylene, and butenylene. Preferred alkynylene groups include, without limitation, ethynylene, propynylene, and butynylene.

The term “azolyl” as employed herein is intended to mean a five-membered saturated or unsaturated heterocyclic group containing two or more hetero-atoms, as ring atoms, selected from the group consisting of nitrogen, sulfur and oxygen, wherein at least one of the hetero-atoms is a nitrogen atom. Preferred azolyl groups include, but are not limited to, optionally substituted imidazolyl, oxazolyl, thiazolyl, pyrazolyl, isoxazolyl, isothiazolyl, 1,3,4-thiadiazolyl, 1,2,4-thiadiazolyl, 1,2,4-oxadiazolyl, and 1,3,4-oxadiazolyl.

The term “carbocycle” as employed herein is intended to mean a cycloalkyl or aryl moiety. The term “carbocycle” also includes a cycloalkenyl moiety having at least one carbon-carbon double bond.

The term “cycloalkyl” is intended to mean a saturated or unsaturated mono-, bi-, tri- or poly-cyclic hydrocarbon group having about 3 to 15 carbons, preferably having 3 to 12 carbons, preferably 3 to 8 carbons, more preferably 3 to 6 carbons, and more preferably still 5 or 6 carbons. In certain preferred embodiments, the cycloalkyl group is fused to an aryl, heteroaryl or heterocyclic group. Preferred cycloalkyl groups include, without limitation, cyclopenten-2-enone, cyclopenten-2-enol, cyclohex-2-enone, cyclohex-2-enol, cyclopropyl, cyclobutyl, cyclobutenyl, cyclopentyl, cyclopentenyl, cyclohexyl, cyclohexenyl, cycloheptyl, cyclooctyl, etc.

The term “heteroalkyl” is intended to mean a saturated or unsaturated, straight chain or branched aliphatic group, wherein one or more carbon atoms in the group are independently replaced by a moiety selected from the group consisting of O, S, N,N-alkyl, —S(O)—, —S(O)₂—, —S(O)₂NH—, or —NHS(O)₂—

The term “aryl” is intended to mean a mono-, bi-, tri- or polycyclic aromatic moiety, preferably a C₆-C₁₄aromatic moiety, preferably comprising one to three aromatic rings. Preferably, the aryl group is a C₆-C₁₀aryl group, more preferably a C₆aryl group. Preferred aryl groups include, without limitation, phenyl, naphthyl, anthracenyl, and fluorenyl.

The terms “aralkyl” or “arylalkyl” are intended to mean a group comprising an aryl group covalently linked to an alkyl group. If an aralkyl group is described as “optionally substituted”, it is intended that either or both of the aryl and alkyl moieties may independently be optionally substituted or unsubstituted. Preferably, the aralkyl group is (C₁-C₆)alk(C₆-C₁₀)aryl, including, without limitation, benzyl, phenethyl, and naphthylmethyl. For simplicity, when written as “arylalkyl” this term, and terms related thereto, is intended to indicate the order of groups in a compound as “aryl-alkyl”. Similarly, “alkyl-aryl” is intended to indicate the order of the groups in a compound as “alkyl-aryl”.

The terms “heterocyclyl”, “heterocyclic” or “heterocycle” are intended to mean a group which is a mono-, bi-, or polycyclic structure having from about 3 to about 14 atoms, wherein one or more atoms are independently selected from the group consisting of N, O, and S. The ring structure may be saturated, unsaturated or partially unsaturated. In certain preferred embodiments, the heterocyclic group is non-aromatic, in which case the group is also known as a heterocycloalkyl. In certain preferred embodiments, the heterocyclic group is a bridged heterocyclic group (for example, a bicyclic moiety with a methylene, ethylene or propylene bridge). In a bicyclic or polycyclic structure, one or more rings may be aromatic; for example one ring of a bicyclic heterocycle or one or two rings of a tricyclic heterocycle may be aromatic, as in indan and 9,10-dihydro anthracene. Preferred heterocyclic groups include, without limitation, epoxy, aziridinyl, tetrahydrofuranyl, pyrrolidinyl, piperidinyl, piperazinyl, thiazolidinyl, oxazolidinyl, oxazolidinonyl, and morpholino. In certain preferred embodiments, the heterocyclic group is fused to an aryl, heteroaryl, or cycloalkyl group. Examples of such fused heterocycles include, without limitation, tetrahydroquinoline and dihydrobenzofuran. Specifically excluded from the scope of this term are compounds where an annular O or S atom is adjacent to another O or S atom.

In certain preferred embodiments, the heterocyclic group is a heteroaryl group. As used herein, the term “heteroaryl” is intended to mean a mono-, bi-, tri- or polycyclic group having 5 to 18 ring atoms, preferably 5 to 14 ring atoms, more preferably 5, 6, 9, or 10 ring atoms; preferably having 6, 10, or 14 pi electrons shared in a cyclic array; and having, in addition to carbon atoms, between one or more heteroatoms selected from the group consisting of N, O, and S. The term “heteroaryl” is also intended to encompass the N-oxide derivative (or N-oxide derivatives, if the heteroaryl group contains more than one nitrogen such that more than one N-oxide derivative may be formed) of a nitrogen-containing heteroaryl group. For example, a heteroaryl group may be pyrimidinyl, pyridinyl, benzimidazolyl, thienyl, benzothiazolyl, benzofuranyl and indolinyl. Preferred heteroaryl groups include, without limitation, thienyl, benzothienyl, furyl, benzofuryl, dibenzofuryl, pyrrolyl, imidazolyl, pyrazolyl, pyridyl, pyrazinyl, pyrimidinyl, indolyl, quinolyl, isoquinolyl, quinoxalinyl, tetrazolyl, oxazolyl, thiazolyl, isoxazolyl, benzo[b]thienyl, naphtha[2,3-b]thianthrenyl, zanthenyl, quinolyl, benzothiazolyl, benzimidazolyl, beta-carbolinyl and perimidinyl. Illustrative examples of N-oxide derivatives of heteroaryl groups include, but are not limited to, pyridyl N-oxide, pyrazinyl N-opxide, pyrimidinyl N-oxide, pyridazinyl N-oxide, triazinyl N-oxide, isoquinolyl N-oxide and quinolyl N-oxide.

The terms “arylene,” “heteroarylene,” or “heterocyclylene” are intended to mean an aryl, heteroaryl, or heterocyclyl group, respectively, as defined hereinabove, that is positioned between and serves to connect two other chemical groups.

A heteroalicyclic group refers specifically to a non-aromatic heterocyclyl radical. A heteroalicyclic may contain unsaturation, but is not aromatic.

A heterocyclylalkyl group refers to a residue in which a heterocyclyl is attached to a parent structure via one of an alkylene, alkylidene, or alkylidyne radical. Examples include (4-methylpiperazin-1-yl)methyl, (morpholin-4-yl)methyl, (pyridine-4-yl)methyl, 2-(oxazolin-2-yl)ethyl, 4-(4-methylpiperazin-1-yl)-2-butenyl, and the like. If a heterocyclylalkyl is described as “optionally substituted” it is meant that both the heterocyclyl and the corresponding alkylene, alkylidene, or alkylidyne radical portion of a heterocyclylalkyl group may be optionally substituted. A “lower heterocyclylalkyl” refers to a heterocyclylalkyl where the “alkyl” portion of the group has one to six carbons.

A heteroalicyclylalkyl group refers specifically to a heterocyclylalkyl where the heterocyclyl portion of the group is non-aromatic.

Preferred heterocyclyls and heteroaryls include, but are not limited to, azepinyl, azetidinyl, acridinyl, azocinyl, benzidolyl, benzimidazolyl, benzofuranyl, benzofurazanyl, benzofuryl, benzothiofuranyl, benzothiophenyl, benzoxazolyl, benzothiazolyl, benzothienyl, benztriazolyl, benztetrazolyl, benzisoxazolyl, benzisothiazolyl, benzimidazolinyl, benzoxazolyl, benzoxadiazolyl, benzopyranyl, carbazolyl, 4aH-carbazolyl, carbolinyl, chromanyl, chromenyl, cinnolinyl, coumarinyl, decahydroquinolinyl, dibenzofuryl, 1,3-dioxolane, 2H,6H-1,5,2-dithiazinyl, dihydrofuro[2,3-b]tetrahydrofuran, dihydroisoindolyl, dihydroquinazolinyl (such as 3,4-dihydro-4-oxo-quinazolinyl), furanyl, furopyridinyl (such as fuor[2,3-c]pyridinyl, furo[3,2-b]pyridinyl or furo[2,3-b]pyridinyl), furyl, furazanyl, hexahydrodiazepinyl, imidazolidinyl, imidazolinyl, imidazolyl, indazolyl, 1H-indazolyl, indolenyl, indolinyl, indolizinyl, indolyl, 3H-indolyl, isobenzofuranyl, isochromanyl, isoindazolyl, isoindolinyl, isoindolyl, isoquinolyl, isoquinolinyl, isothiazolidinyl, isothiazolyl, isoxazolinyl, isoxazolyl, methylenedioxyphenyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxazolidinyl, oxetanyl, 2-oxoazepinyl, 2-oxopiperazinyl, 2-oxopiperidinyl, 2-oxopyrrolodinyl, pyrimidinyl, phenanthridinyl, phenanthrolinyl, phenazinyl, phenothiazinyl, phenoxathiinyl, phenoxazinyl, phthalazinyl, piperazinyl, piperidinyl, piperidonyl, 4-piperidonyl, piperonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, pyrrolopyridyl, 2H-pyrrolyl, pyrrolyl, quinazolinyl, quinolyl, quinolinyl, 4H-quinolizinyl, quinoxalinyl, quinuclidinyl, tetrahydro-1,1-dioxothienyl, tetrahydrofuranyl, tetrahydrofuryl, tetrahydroisoquinolinyl, tetrahydroquinolinyl, tetrahydropyranyl, tetrazolyl, thiazolidinyl, 6H-1,2,5-thiadiazinyl, thiadiazolyl (e.g., 1,2,3-thiadiazolyl, 1,2,4-thiadiazolyl, 1,2,5-thiadiazolyl, 1,3,4-thiadiazolyl), thiamorpholinyl, thiamorpholinyl sulfoxide, thiamorpholuiyl sulfone, thianthrenyl, thiazolyl, thienyl, thienothiazolyl, thienooxazolyl, thienoimidazolyl, thiophenyl, triazinyl, triazinylazepinyl, triazolyl (e.g., 1,2,3-triazolyl, 1,2,4-triazolyl, 1,2,5-triazolyl, 1,3,4-triazolyl), and xanthenyl.

A “halohydrocarbyl” as employed herein is a hydrocarbyl moiety, in which from one to all hydrogens have been replaced with an independently selected halo.

As employed herein, and unless stated otherwise, when a moiety (e.g., alkyl, heteroalkyl, cycloalkyl, aryl, heteroaryl, heterocyclyl, etc.) is described as “optionally substituted” it is meant that the group optionally has from one to four, preferably from one to three, more preferably one or two, independently selected non-hydrogen substituents. Suitable substituents include, without limitation, halo, hydroxy, oxo (e.g., an annular —CH— substituted with oxo is —C(O)—) nitro, halohydrocarbyl, hydrocarbyl, alkyl, cycloalkyl, heterocyclyl, aryl, heteroaryl, aralkyl, alkoxy, aryloxy, amino, acylamino, alkylcarbamoyl, arylcarbamoyl, aminoalkyl, acyl, carboxy, hydroxyalkyl, alkanesulfonyl, arenesulfonyl, alkanesulfonamido, arenesulfonamido, aralkylsulfonamido, alkylcarbonyl, acyloxy, cyano, and ureido groups. Preferred substituents, which are themselves not further substituted (unless expressly stated otherwise) are:
(a) halo, hydroxy, cyano, oxo, carboxy, formyl, nitro, amino, amidino, guanidino,
(b) C₁-C₅alkyl or alkenyl or arylalkyl imino, carbamoyl, azido, carboxamido, mercapto, hydroxy, hydroxyalkyl, alkylaryl, arylalkyl, C₁-C₈alkyl, C₁-C₈alkenyl, C₁-C₈alkoxy, C₁-C₈alkyamino, C₁-C₈alkoxycarbonyl, aryloxycarbonyl, C₂-C₈acyl, —C(O)—N(R³⁰)-alkyl-cycloalkyl, —C(O)—N(R³⁰)-alkyl-heterocyclyl, —C(O)—N(R³⁰)-alkyl-aryl, —C(O)—N(R³⁰)-alkyl-heteroaryl, —C(O)-cycloalkyl, —C(O)-heterocyclyl, —C(O)-aryl, —C(O)-heteroaryl, C₂-C₈acylamino, C₁-C₈alkylthio, arylalkylthio, arylthio, C₁-C₈alkylsulfinyl, arylalkylsulfinyl, arylsulfinyl, C₁-C₈alkylsulfonyl, arylalkylsulfonyl, arylsulfonyl, C₀-C₆N-alkyl carbamoyl, C₂-C₁₅N,N-dialkylcarbamoyl, C₃-C₇ cycloalkyl, aroyl, aryloxy, arylalkyl ether, aryl, aryl fused to a cycloalkyl or heterocycle or another aryl ring, C₃-C₇heterocycle, C₅-C₁₅heteroaryl or any of these rings fused or spiro-fused to a cycloalkyl, heterocyclyl, or aryl, wherein each of the foregoing is further optionally substituted with one more moieties listed in (a), above; and
(c) —(CR³²R³³)ₛ—NR³⁰R³¹, wherein s is from 0 (in which case the nitrogen is directly bonded to the moiety that is substituted) to 6, R³² and R³³ are each independently hydrogen, halo, hydroxyl or C₁-C₄alkyl, and R³⁰ and R³¹ are each independently hydrogen, cyano, oxo, hydroxyl, C₁-C₈alkyl, C₁-C₈heteroalkyl, C₁-C₈alkenyl, carboxamido, C₁-C₃alkyl-carboxamido, carboxamido-C₁-C₃alkyl, amidino, C₂-C₈hydroxyalkyl, C₁-C₃alkylaryl, aryl-C₁-C₃alkyl, C₁-C₃alkylheteroaryl, heteroaryl-C₁-C₃alkyl, C₁-C₃alkylheterocyclyl, heterocyclyl-C₁-C₃alkyl C₁-C₃alkylcycloalkyl, cycloalkyl-C₁-C₃alkyl, C₂-C₈alkoxy, C₂-C₈alkoxy-C₁-C₄alkyl, C₁-C₈alkoxycarbonyl, aryloxycarbonyl, aryl-C₁-C₃alkoxycarbonyl, heteroaryloxycarbonyl, heteroaryl-C₁-C₃alkoxycarbonyl, C₁-C₈acyl, C₀-C₈alkyl-carbonyl, aryl-C₀-C₈alkyl-carbonyl, heteroaryl-C₀-C₈alkyl-carbonyl, cycloalkyl-C₀-C₈alkyl-carbonyl, heterocyclyl-C₀-C₈alkyl-carbonyl, C₀-C₈alkyl-NH-carbonyl, aryl-C₀-C₈alkyl-NH-carbonyl, heteroaryl-C₀-C₈alkyl-NH-carbonyl, cycloalkyl-C₀-C₈alkyl-NH-carbonyl, heterocylclyl-C₀-C₈alkyl-NH-carbonyl, cycloalkyl-S(O)₂—, heterocyclyl-S(O)₂—, aryl-S(O)₂—, heteroaryl-S(O)₂—, C₀-C₈alkyl-O-carbonyl, aryl-C₀-C₈alkyl-O-carbonyl, heteroaryl-C₀-C₈alkyl-O-carbonyl, cycloalkyl-C₀-C₈alkyl-O-carbonyl, heterocyclyl-C₀-C₈alkyl-O-carbonyl, C₁-C₈alkylsulfonyl, arylalkylsulfonyl, arylsulfonyl, heteroarylalkylsulfonyl, heteroarylsulfonyl, C₁-C₈alkyl-NH-sulfonyl, arylalkyl-NH-sulfonyl, aryl-NH-sulfonyl, heteroarylalkyl-NH-sulfonyl, heteroaryl-NH-sulfonyl aroyl, aryl, cycloalkyl, heterocyclyl, heteroaryl, aryl-C₁-C₃alkyl-, cycloalkyl-C₁-C₃alkyl-, heterocyclyl-C₁-C₃alkyl-, heteroaryl-C₁-C₃alkyl-, or a protecting group, wherein each of the foregoing is further optionally substituted with one more moieties listed in (a), above; or
   R³⁰ and R³¹ taken together with the N to which they are attached form a heterocyclyl or heteroaryl, each of which is optionally substituted with from 1 to 3 substituents selected from the group consisting of (a) above, a protecting group, and (X³⁰—Y³¹—), wherein said heterocyclyl may also be bridged (forming a bicyclic moiety with a methylene, ethylene or propylene bridge); wherein
   X³⁰ is selected from the group consisting of H, C₁-C₈alkyl, C₂-C₈alkenyl-, C₂-C₈alkynyl-, —C₀-C₃alkyl-C₂-C₅alkenyl-C₀-C₃alkyl, C₀-C₃alkyl-C₂-C₅alkynyl-C₀-C₃alkyl, C₀-C₃alkyl-O—C₀-C₃alkyl-, HO—C₀-C₃alkyl-, C₀-C₄alkyl-N(R³⁰)—C₀-C₃alkyl-, N(R³⁰)(R³¹)—C₀-C₃alkyl-, N(R³⁰)(R³¹)—C₀-C₃alkenyl-, N(R³⁰)(R³¹)—C₀-C₃alkynyl-, (N(R³⁰)(R³¹))₂—C═N—, C₀-C₃alkyl-S(O)₀₋₂—C₀-C₃alkyl-, CF₃—C₀-C₃alkyl-, C₁-C₈heteroalkyl, aryl, cycloalkyl, heterocyclyl, heteroaryl, aryl-C₁-C₃alkyl-, cycloalkyl-C₁-C₃alkyl-, heterocyclyl-C₁-C₃alkyl-, heteroaryl-C₁-C₃alkyl-, N(R³⁰)(R³¹)-heterocyclyl-C₁-C₃alkyl-, wherein the aryl, cycloalkyl, heteroaryl and heterocycyl are optionally substituted with from 1 to 3 substituents from (a); and
   Y³¹ is selected from the group consisting of a direct bond, —O—, —N(R³⁰)—, —C(O)—, —O—C(O)—, —C(O)—O—, —N(R³⁰)—C(O)—, —C(O)—N(R³⁰)—, —N(R³⁰)—C(S)—, —C(S)—N(R³⁰)—, —N(R³⁰)—C(O)—N(R³¹)—, —N(R³⁰)—C(NR³⁰)—N(R³¹)—, —N(R³⁰)—C(NR³¹)—, —C(NR³¹)—N(R³⁰)—, —N(R³⁰)—C(S)—N(R³¹)—, —N(R³⁰)—C(O)—O—, —O—C(O)—N(R³¹)—, —N(R³⁰)—C(S)—O—, —O—C(S)—N(R³¹)—, —S(O)₀₋₂—, —SO₂N(R³¹)—, —N(R³¹)—SO₂— and —N(R³⁰)—SO₂N(R³¹)—.

A moiety that is substituted is one in which one or more (preferably one to four, preferably from one to three and more preferably one or two), hydrogens have been independently replaced with another chemical substituent. As a non-limiting example, substituted phenyls include 2-fluorophenyl, 3,4-dichlorophenyl, 3-chloro-4-fluoro-phenyl, 2-fluoro-3-propylphenyl. As another non-limiting example, substituted n-octyls include 2,4-dimethyl-5-ethyl-octyl and 3-cyclopentyl-octyl. Included within this definition are methylenes (—CH₂—) substituted with oxygen to form carbonyl —CO—.

When there are two optional substituents bonded to adjacent atoms of a ring structure, such as for example a phenyl, thiophenyl, or pyridinyl, the substituents, together with the atoms to which they are bonded, optionally form a 5- or 6-membered cycloalkyl or heterocycle having 1, 2, or 3 annular heteroatoms.

In a preferred embodiment, a group, such as a hydrocarbyl, heteroalkyl, heterocyclic and/or aryl group is unsubstituted.

In other preferred embodiments, a group, such as a hydrocarbyl, heteroalkyl, heterocyclic and/or aryl group is substituted with from 1 to 4 (preferably from one to three, and more preferably one or two) independently selected substituents.

Preferred substituents on alkyl groups include, but are not limited to, hydroxyl, halogen (e.g., a single halogen substituent or multiple halo substituents; in the latter case, groups such as —CF₃ or an alkyl group bearing Cl₃), oxo, cyano, nitro, alkyl, cycloalkyl, alkenyl, cycloalkenyl, alkynyl, heterocycle, aryl, —OR^{a}, —SR^{a}, —S(═O)R^{e}, —S(═O)₂R^{e}, —P(═O)₂R^{e}, —S(═O)₂OR^{e}, —P(═O)₂OR^{e}, —NR^{b}R^{c}, —NR^{b}S(═O)₂R^{e}, —NR^{b}P(═O)₂R^{e}, —S(═O)₂NR^{b}R^{c}, —P(═O)₂NR^{b}R^{c}, —C(═O)OR^{e}, —C(═O)R^{a}, —C(═O)NR^{b}R^{c}, —OC(═O)R^{a}, —OC(═O)NR^{b}R^{c}, —NR^{b}C(═O)OR^{e}, —NR^{d}C(═O)NR^{b}R^{c}, —NR^{d}S(═O)₂NR^{b}R^{c}, —NR^{d}P(═O)₂NR^{b}R^{c}, —NR^{b}C(═O)R^{a} or —NR^{b}P(═O)₂R^{e}, wherein R^{a} is hydrogen, alkyl, cycloalkyl, alkenyl, cycloalkenyl, alkynyl, heterocycle or aryl; R^{b}, R^{c} and R^{d} are independently hydrogen, alkyl, cycloalkyl, heterocycle or aryl, or said R^{b} and R^{c} together with the N to which they are bonded optionally form a heterocycle; and R^{e} is alkyl, cycloalkyl, alkenyl, cycloalkenyl, alkynyl, heterocycle or aryl. In the aforementioned exemplary substituents, groups such as alkyl, cycloalkyl, alkenyl, alkynyl, cycloalkenyl, heterocycle and aryl can themselves be optionally substituted.

Preferred substituents on alkenyl and alkynyl groups include, but are not limited to, alkyl or substituted alkyl, as well as those groups recited as preferred alkyl substituents.

Preferred substituents on cycloalkyl groups include, but are not limited to, nitro, cyano, alkyl or substituted alkyl, as well as those groups recited about as preferred alkyl substituents. Other preferred substituents include, but are not limited to, spiro-attached or fused cyclic substituents, preferably spiro-attached cycloalkyl, spiro-attached cycloalkenyl, spiro-attached heterocycle (excluding heteroaryl), fused cycloalkyl, fused cycloalkenyl, fused heterocycle, or fused aryl, where the aforementioned cycloalkyl, cycloalkenyl, heterocycle and aryl substituents can themselves be optionally substituted.

Preferred substituents on cycloalkenyl groups include, but are not limited to, nitro, cyano, alkyl or substituted alkyl, as well as those groups recited as preferred alkyl substituents. Other preferred substituents include, but are not limited to, spiro-attached or fused cyclic substituents, especially spiro-attached cycloalkyl, spiro-attached cycloalkenyl, spiro-attached heterocycle (excluding heteroaryl), fused cycloalkyl, fused cycloalkenyl, fused heterocycle, or fused aryl, where the aforementioned cycloalkyl, cycloalkenyl, heterocycle and aryl substituents can themselves be optionally substituted.

Preferred substituents on aryl groups include, but are not limited to, nitro, cycloalkyl or substituted cycloalkyl, cycloalkenyl or substituted cycloalkenyl, cyano, alkyl or substituted alkyl, as well as those groups recited above as preferred alkyl substituents. Other preferred substituents include, but are not limited to, fused cyclic groups, especially fused cycloalkyl, fused cycloalkenyl, fused heterocycle, or fused aryl, where the aforementioned cycloalkyl, cylcoalkenyl, heterocycle and aryl substituents can themselves be optionally substituted. Still other preferred substituents on aryl groups (phenyl, as a non-limiting example) include, but are not limited to, haloalkyl and those groups recited as preferred alkyl substituents.

Preferred substituents on heterocylic groups include, but are not limited to, cycloalkyl, substituted cycloalkyl, cycloalkenyl, substituted cycloalkenyl, nitro, oxo (i.e., ═O), cyano, alkyl, substituted alkyl, as well as those groups recited as preferred alkyl substituents. Other preferred substituents on heterocyclic groups include, but are not limited to, spiro-attached or fused cylic substituents at any available point or points of attachment, more preferably spiro-attached cycloalkyl, spiro-attached cycloalkenyl, spiro-attached heterocycle (excluding heteroaryl), fused cycloalkyl, fused cycloakenyl, fused heterocycle and fused aryl, where the aforementioned cycloalkyl, cycloalkenyl, heterocycle and aryl substituents can themselves be optionally substituted.

In certain preferred embodiments, a heterocyclic group is substituted on carbon, nitrogen and/or sulfur at one or more positions. Preferred substituents on carbon include those groups recited as preferred alkyl substituents. Preferred substituents on nitrogen include, but are not limited to alkyl, aryl, aralkyl, alkylcarbonyl, alkylsulfonyl, arylcarbonyl, arylsulfonyl, alkoxycarbonyl, or aralkoxycarbonyl. Preferred substituents on sulfur include, but are not limited to, oxo and C₁₋₆alkyl. In certain preferred embodiments, nitrogen and sulfur heteroatoms may independently be optionally oxidized and nitrogen heteroatoms may independently be optionally quaternized.

Especially preferred substituents on ring groups, such as aryl, heteroaryl, cycloalkyl and heterocyclyl, include halogen, alkoxy and alkyl.

Especially preferred substituents on alkyl groups include halogen and hydroxy.

Preferred substituents on aromatic polycycles including, but not limited to, naphthyl and quinoline, include C₁-C₆alkyl, cycloalkylalkyl (e.g. cyclopropylmethyl), oxyalkyl, halo, nitro, amino, alkylamino, aminoalkyl, alkyl ketones, nitrile, carboxyalkyl, alkylsulfonyl, arylsulfonyl, aminosulfonyl and OR^{aa}, such as alkoxy, wherein R^{aa} is selected from the group consisting of H, C₁-C₆alkyl, C₄-C₉cycloalkyl, C₄-C₉heterocycloalkyl, aryl, heteroaryl, arylalkyl, heteroarylalkyl and —(CH₂)₀₋₆Z^{a}R^{bb}, wherein Z^{a} is selected from the group consisting of O, NR^{cc}, S and S(O), and R^{bb} is selected from the group consisting of H, C₁-C₆alkyl, C₄-C₉cycloalkyl, C₄-C₉heterocycloalkyl, C₄-C₉heterocycloalkylalkyl, aryl, mixed aryl and non-aryl polycycle, heteroaryl, arylalkyl, (e.g. benzyl), and heteroarylalkyl (e.g. pyridylmethyl); and R^{cc} is selected from the group consisting of H, C₁-C₆alkyl, C₄-C₉cycloalkyl, C₄-C₉heterocycloalkyl, aryl, heteroaryl, arylalkyl (e.g. benzyl), heteroarylalkyl (e.g. pyridylmethyl) and amino acyl.

Preferred non-aromatic polycycles include, but are not limited to, bicyclic and tricyclic fused ring systems where each ring can be 4-9 membered and each ring can contain zero, 1 or more double and/or triple bonds. Suitable examples of non-aromatic polycycles include, but are not limited to, decalin, octahydroindene, perhydrobenzocycloheptene and perhydrobenzo-[f]-azulene. Such groups are optionally substituted with for example, but not limited to, C₃-C₉cycloalkyl groups, such as cyclopropyl, cyclobutyl, cyclopentyl, cyclohexyl and the like. Unless otherwise noted, non-aromatic polycycles include both unsubstituted cycloalkyl groups and cycloalkyl groups that are substituted by one or more suitable substituents, including but not limited to, C₁-C₆alkyl, halo, hydroxy, aminoalkyl, oxyalkyl, alkylamino and OR^{aa}, such as alkoxy. Preferred substituents for such cycloalkyl groups include halo, hydroxy, alkoxy, oxyalkyl, alkylamino and aminoalkyl.

Preferred mixed aryl and non-aryl polycycles include bicyclic and tricylic fused ring systems where each ring can be 4-9 membered and at least one ring is aromatic. Suitable examples of mixed aryl and non-aryl polycycles include methylenedioxyphenyl, bis-methylenedioxyphenyl, 1,2,3,4-tetrahydronaphthalene, dibenzosuberane dihydroanthracene and 9H-fluorene. Such groups are unsubstituted or substituted by nitro or as described above for non-aromatic polycycles.

Polyheteroaryls include bicyclic and tricyclic fused rings systems where each ring can independently be 5 or 6 membered and contain one or more heteroatom, for example, 1, 2, 3 or 4 heteroatoms, chosen from O, N or S such that the fused ring system is aromatic. Suitable examples of polyheteroaryl ring systems include quinoline, isoquinoline, pyridopyrazine, pyrrolopyridine, furopyridine, indole, benzofuran, benzothiofuran, benzindole, benzoxazole, pyrroloquinoline, and the like. Unless otherwise noted, polyheteroaryls are unsubstituted or substituted on a carbon atom by one or more suitable substituents, including but not limited to, straight and branched optionally substituted C₁-C₆alkyl, unsaturation (i.e., there are one or more double or triple C—C bonds), acyl, cycloalkyl, halo, oxyalkyl, alkylamino, aminoalkyl, acylamino and OR^{aa}, for example alkoxy, and a substituent of the formula —O—(CH₂CH═CH(CH₃)(CH₂))₁₋₃H. Examples of suitable straight and branched C₁-C₆alkyl substituents include but are not limited to methyl, ethyl, n-propyl, 2-propyl, n-butyl, sec-butyl, t-butyl and the like. Preferred substituents include halo, hydroxy, alkoxy, oxyalkyl, alkylamino and aminoalkyl. Nitrogen atoms are unsubstituted or substituted, for example by R^{cc}. Preferred substituents on such nitrogen atoms include H, C₁-C₄alkyl, acyl, aminoacyl and sulfonyl.

Preferred non-aromatic polyheterocyclics include but are not limited to bicyclic and tricyclic ring systems where each ring can be 4-9 membered, contain one or more heteroatom, for example 1, 2, 3 or 4 heteroatoms, chosen from O, N or S and contain zero, or one or more C—C double or triple bonds. Suitable examples of non-aromatic polyheterocycles include but are not limited to, hexitol, cis-perhydro-cyclohepta[b]pyridinyl, decahydro-benzo[f][1,4]oxazepinyl, 2,8-dioxabicyclo[3.3.0]octane, hexahydro-thieno[3,2-b]thiophene, perhydropyrrolo[3,2-b]pyrrole, perhydronaphthyridine, perhydrop-1H-dicyclopenta[b,e]pyran. Unless otherwise noted, non-aromatic polyheterocyclics are unsubstituted or substituted on a carbon atom by one or more substituents, including but not limited to straight and branched optionally substituted C₁-C₆alkyl, unsaturation (i.e., there are one or more double or triple C—C bonds), acyl, cycloalkyl, halo, oxyalkyl, alkylamino, aminoalkyl, acylamino and OR^{aa}, for example alkoxy. Examples of suitable straight and branched C₁-C₆alkyl substituents include but are not limited to methyl, ethyl, n-propyl, 2-propyl, n-butyl, sec-butyl, t-butyl and the like. Preferred substituents include halo, hydroxy, alkoxy, oxyalkyl, alkylamino and aminoalkyl. Nitrogen atoms are unsubstituted are substituted, for example, by R^{cc}. Preferred N substituents include H, C₁-C₄ alkyl, acyl, aminoacyl and sulfonyl.

Preferred mixed aryl and non-aryl polyheterocycles include but are not limited to bicyclic and tricyclic fused ring systems where each ring can be 4-9 membered, contain one or more heteroatom chosen from O, N or S and at least one of the rings must be aromatic. Suitable examples of mixed aryl and non-aryl polyheteorcycles include 2,3-dihydroindole, 1,2,3,4-tetrahydroquinoline, 5,11-dihydro-10H-dibenz[b,e][1,4]diazepine, 5H-dibenzo[b,e][1,4]diazepine, 1,2-dihydropyrrolo[3,4-b][1,5]benzodiazepine, 1,5-dihydropyrido[2,3-b][1,4]diazepin-4-one, 1,2,3,4,6,11-hexhydro-benzo[b]pyrido[2,3-e][1,4]diazepine-5-one. Unless otherwise noted, mixed aryl and non-aryl polyheterocyclics are unsubstituted or substituted on a carbon atom by one or more suitable substituents including but not limited to —N—OH, ═N—OH, optionally substituted alkyl unsaturation (i.e., there are one or more double or triple C—C bonds), acyl, cycloalkyl, halo, oxyalkyl, alkylamino, aminoalkyl, acylamino and OR^{aa}, for example alkoxy. Nitrogen atoms are unsubstituted or substituted, for example, by R^{cc}. Preferred N substituents include H, C₁₋₄alkyl, acyl aminoacyl and sulfonyl.

The term “halogen” or “halo” as employed herein refers to chlorine, bromine, fluorine, or iodine. As herein employed, the term “acyl” refers to an alkylcarbonyl or arylcarbonyl substituent. The term “acylamino” refers to an amide group attached at the nitrogen atom (i.e., R—CO—NH—). The term “carbamoyl” refers to an amide group attached at the carbonyl carbon atom (i.e., NH₂—CO—). The nitrogen atom of an acylamino or carbamoyl substituent is additionally optionally substituted. The term “sulfonamido” refers to a sulfonamide substituent attached by either the sulfur or the nitrogen atom. The term “amino” is meant to include NH₂, alkylamino, di-alkyl-amino, arylamino, and cyclic amino groups. The term “ureido” as employed herein refers to a substituted or unsubstituted urea moiety.

The term “radical” as used herein means a chemical moiety comprising one or more unpaired electrons.

Where optional substituents are chosen from “one or more” groups it is to be understood that this definition includes all substituents being chosen from one of the specified groups or the substituents being chosen from two or more of the specified groups.

In addition, substituents on cyclic moieties (i.e., cycloalkyl, heterocyclyl, aryl, heteroaryl) include 5- to 6-membered mono- and 9- to 14-membered bi-cyclic moieties fused to the parent cyclic moiety to form a bi- or tri-cyclic fused ring system. Substituents on cyclic moieties also include 5- to 6-membered mono- and 9- to 14-membered bi-cyclic moieties attached to the parent cyclic moiety by a covalent bond to form a bi- or tri-cyclic bi-ring system. For example, an optionally substituted phenyl includes, but is not limited to, the following:

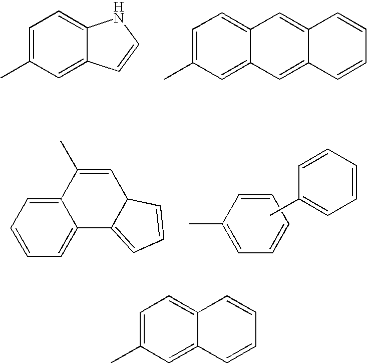

A saturated or unsaturated three- to eight-membered carbocyclic ring is preferably a four- to seven-membered, more preferably five- or six-membered, saturated or unsaturated carbocyclic ring. Examples of saturated or unsaturated three- to eight-membered carbocyclic rings include phenyl, cyclopropyl, cyclobutyl, cyclopentyl, cyclohexyl, and cycloheptyl.

A saturated or unsaturated three- to eight-membered heterocyclic ring contains at least one heteroatom selected from oxygen, nitrogen, and sulfur atoms. The saturated or unsaturated three- to eight-membered heterocyclic ring preferably contains one or two heteroatoms with the remaining ring-constituting atoms being carbon atoms. The saturated or unsaturated three- to eight-membered heterocyclic ring is preferably a saturated or unsaturated four- to seven-membered heterocyclic ring, more preferably a saturated or unsaturated five- or six-membered heterocyclic ring. Examples of saturated or unsaturated three- to eight-membered heterocyclic groups include thienyl, pyridyl, 1,2,3-triazolyl, imidazolyl, isoxazolyl, pyrazolyl, piperazinyl, piperazino, piperidyl, piperidino, morpholinyl, morpholino, homopiperazinyl, homopiperazino, thiomorpholinyl, thiomorpholino, tetrahydropyrrolyl, and azepanyl.

A saturated or unsaturated carboxylic and heterocyclic group may condense with another saturated or heterocyclic group to form a bicyclic group, preferably a saturated or unsaturated nine- to twelve-membered bicyclic carbocyclic or heterocyclic group. Bicyclic groups include naphthyl, quinolyl, 1,2,3,4-tetrahydroquinolyl, 1,4-benzoxanyl, indanyl, indolyl, and 1,2,3,4-tetrahydronaphthyl.

When a carbocyclic or heterocyclic group is substituted by two C₁₋₆ alkyl groups, the two alkyl groups may combine together to form an alkylene chain, preferably a C₁₋₃ alkylene chain. Carbocyclic or heterocyclic groups having this crosslinked structure include bicyclo[2.2.2]octanyl and norbornanyl.

The terms “protect”, “protected”, and “protecting” are intended to refer to a process in which a functional group in a chemical compound is selectively masked by a non-reactive functional group in order to allow a selective reaction(s) to occur elsewhere on said chemical compound. Such non-reactive functional groups are herein termed “protecting groups”. For example, the term “nitrogen protecting group”, is intended to mean a group capable of selectively masking the reactivity of a nitrogen (N) group. The term “suitable protecting group” is intended to mean a protecting group useful in the preparation of the compounds of the present invention. Such groups are generally able to be selectively introduced and removed using mild reaction conditions that do not interfere with other portions of the subject compounds. Protecting groups that are suitable for use in the processes and methods of the present invention are well known, such as but not limited to, Bn- (or —CH₂Ph), —CHPh₂, alloc (or CH₂═CH—CH₂—O—C(O)—), BOC-, -Cbz (or Z-), —F-moc, —C(O)—CF₃, N-Phthalimide, 1-Adoc-, TBDMS-, TBDPS-, TMS-, TIPS—, IPDMS-, —SiR₃, SEM-, t-Bu-, Tr-, THP- and Allyl-. These protecting groups may be removed at a convenient stage using methods known from the art. The chemical properties of such protecting groups, methods for their introduction and their removal art known in the art and can be found for example in T. Greene and P. Wuls, Protective Groups in Organic Synthesis (3rd ed.), John Wiley & Sons, NY (1999), herein incorporated by reference in its entirety. The terms “deprotect”, “deprotected”, and “deprotecting” are intended to refer to the process of removing a protecting group from a compound.

Some compounds of the invention may have chiral centers and/or geometric isomeric centers (E- and Z-isomers), and it is to be understood that the invention encompasses all such optical, enantiomeric, diastereoisomeric and geometric isomers. The invention also comprises all tautomeric forms of the compounds disclosed herein. Where compounds of the invention include chiral centers, the invention encompasses the enantiomerically and/or diasteromerically pure isomers of such compounds, the enantiomerically and/or diastereomerically enriched mixtures of such compounds, and the racemic and scalemic mixtures of such compounds. For example, a composition may include a mixture of enantiomers or diastereomers of a compound of formula (I) in at least about 30% diastereomeric or enantiomeric excess. In certain embodiments of the invention, the compound is present in at least about 50% enantiomeric or diastereomeric excess, in at least about 80% enantiomeric or diastereomeric excess, or even in at least about 90% enantiomeric or diastereomeric excess. In certain more preferred embodiments of the invention, the compound is present in at least about 95%, even more preferably in at least about 98% enantiomeric or diastereomeric excess, and most preferably in at least about 99% enantiomeric or diastereomeric excess.

The chiral centers of the present invention may have the S or R configuration. The racemic forms can be resolved by physical methods, such as, for example, fractional crystallization, separation or crystallization of diastereomeric derivates or separation by chiral column chromatography. The individual optical isomers can be obtained either starting from chiral precursors/intermediates or from the racemates by any suitable method, including without limitation, conventional methods, such as, for example, salt formation with an optically active acid followed by crystallization.

The term “prodrug” is intended to mean a derivative of a compound of the present invention that requires a transformation, for example, within the body, to release the active compound. Prodrugs are frequently, although not necessarily, pharmacologically inactive until converted to the parent compound. A hydroxyl containing compound may be converted to, for example, a sulfonate, ester or carbonate prodrug, which may be hydrolyzed in vivo to provide the hydroxyl compound. An amino containing compound may be converted, for example, to a carbamate, amide, enamine, imine, N-phosphonyl, N-phosphoryl or N-sulfenyl prodrug, which may be hydrolyzed in vivo to provide the amino compound. A carboxylic acid compound may be converted to an ester (including silyl esters and thioesters), amide or hydrazide prodrug, which be hydrolyzed in vivo to provide the carboxylic acid compound. Prodrugs for drugs which have functional groups different than those listed above are well known to the skilled artisan. Additionally, prodrugs can be converted to the compounds of the present invention by chemical or biochemical methods in an ex vivo environment. For example, prodrugs can be slowly converted to the compounds of the present invention when placed in a transdermal patch reservoir with a suitable enzyme or chemical reagent.

Typically, in a prodrug, a polar functional group (e.g., a carboxylic acid, an amino group, a hydroxyl group, etc.) is masked by a promoiety, which is labile under physiological conditions. “Promoiety” refers to a form of protecting group that when used to mask a functional group within a compound molecule converts the drug into a prodrug. Typically, the promoiety will be attached to the compound via bond(s) that are cleaved by enzymatic or non-enzymatic means in vivo.

For example, the compounds of the invention may be in a form as is or in the form of an in vivo hydrolyzable ester or in vivo hydrolyzable amide. An in vivo hydrolyzable ester of a compound of the invention containing carboxy or hydroxy group is, for example, a pharmaceutically acceptable ester which is hydrolyzed in the human or animal body to produce the parent acid or alcohol. Suitable pharmaceutically acceptable esters for carboxy include C₁₋₆-alkoxymethyl esters (e.g., methoxymethyl), C₁₋₆-alkanoyloxymethyl esters (e.g., for example pivaloyloxymethyl), phthalidyl esters, C₃₋₈-cycloalkoxycarbonyloxyC₁₋₆-alkyl esters (e.g., 1-cyclohexylcarbonyloxyethyl); 1,3-dioxolen-2-onylmethyl esters (e.g., 5-methyl-1,3-dioxolen-2-onylmethyl; and C₁₋₆-alkoxycarbonyloxyethyl esters (e.g., 1-methoxycarbonyloxyethyl) and may be formed at any carboxy group in the compounds of this invention.

An in vivo hydrolyzable ester of a compound of the invention containing a hydroxy group includes inorganic esters such as phosphate esters and a-acyloxyalkyl ethers and related compounds which as a result of the in vivo hydrolysis of the ester breakdown to give the parent hydroxy group. Examples of α-acyloxyalkyl ethers include acetoxymethoxy and 2,2-dimethylpropionyloxy-methoxy. A selection of in vivo hydrolyzable ester forming groups for hydroxy include alkanoyl, benzoyl, phenylacetyl and substituted benzoyl and phenylacetyl, alkoxycarbonyl (to give alkyl carbonate esters), dialkylcarbamoyl and N—(N,N-dialkylaminoethyl)-N-alkylcarbamoyl (to give carbamates), N,N-dialkylaminoacetyl and carboxyacetyl. Examples of substituents on benzoyl include morpholino and piperazino linked from a ring nitrogen atom via a methylene group to the 3- or 4-position of the benzoyl ring. A suitable value for an in vivo hydrolyzable amide of a compound of the invention containing a carboxy group is, for example, a N—C₁₋₆-alkyl or N,N-di-C₁₋₆-alkyl amide such as N-methyl, N-ethyl, N-propyl, N,N-dimethyl, N-ethyl-N-methyl or N,N-diethyl amide.

Upon administration to a subject, the prodrug undergoes chemical conversion by metabolic or chemical processes to yield a compound of the present invention, or a salt and/or solvate thereof. Solvates of the compounds of the present invention include, for example, hydrates.

Throughout the specification, preferred embodiments of one or more chemical substituents are identified. Also preferred are combinations of preferred embodiments. For example, the invention describes preferred embodiments of L in the compounds and describes preferred embodiments of group Y. Thus, as an example, also contemplated as within the scope of the invention are compounds in which preferred examples of L are as described and in which preferred examples of group Y are as described.

The foregoing merely summarizes one aspect and embodiments of the invention and is not intended to be limiting in nature. This aspect and embodiments are described more fully below.

Compounds

In one aspect, the invention provides compounds of the formula (I), and racemic and scalemic mixtures, diastereomers and enantiomers thereof: 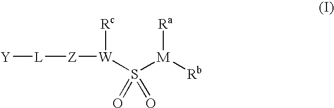
and N-oxides, hydrates, solvates, pharmaceutically acceptable salts, prodrugs and complexes thereof, wherein
M is nitrogen or oxygen; wherein when M is oxygen, R^{b} is absent and W is nitrogen;
W is nitrogen or oxygen; wherein when W is oxygen, R^{c} is absent and M is nitrogen;
R^{a} is independently selected from the group consisting of —H, —C₁-C₆alkyl, a protecting group, —C₁-C₆alkyl-aryl, aryl, —C₁-C₆alkyl-heteroaryl, heteroaryl, —C₁-C₆alkyl-cycloalkyl, cycloalkyl, —C₁-C₆alkyl-heterocyclyl, heterocyclyl, —C(O)—O—C₁-C₆alkyl, —C(O)—O—C₁-C₆alkyl-heterocyclyl, —C(O)—O—C₁-C₆alkyl-alkenyl, —C(O)—O—C₁-C₆alkyl-aryl, —CO—CF₃ and 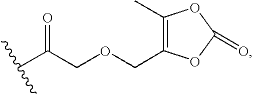
   each of which is optionally substituted; and
when M is nitrogen, R^{a} is further selected from —C(O)—H;
R^{b} and R^{c}, when present, are independently selected from the group consisting of —H, —OH, —CN, —O-alkyl, —C₁-C₆alkyl, —C(O)-alkyl, —NH₂, —NH-alkyl, —C(O)—H, a protecting group, —C₁-C₆alkyl-aryl, aryl, —C₁-C₆alkyl-heteroaryl, -heteroaryl, —C₁-C₆alkyl-cycloalkyl, cycloalkyl, —C₁-C₆alkyl-heterocyclyl, heterocyclyl, —C(O)—C₀-C₃alkyl-aryl, —C(O)—C₀-C₃alkyl-heteroaryl, —C(O)—C₀-C₃alkyl-cycloalkyl, —C(O)—C₀-C₃alkyl-heterocyclyl, —C(O)—O—C₁-C₆alkyl, —C(O)—O—C₁-C₆alkyl-heterocyclyl, —C(O)—O—C₁-C₆alkyl-alkenyl, —C(O)—O—C₁-C₆alkyl-aryl, —CO—CF₃ and 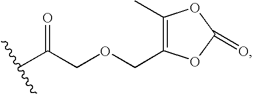
   each of which is optionally substituted;
wherein, when M is nitrogen, R^{a} and R^{b} together with the nitrogen atom to which they are attached optionally form a 3 to 9-membered heterocyclyl, heteroaryl, heterocyclyl-aryl or heterocyclyl-heteroaryl, each of which is optionally substituted; and
wherein, when R^{a} is —C₁-C₆alkyl, —C₁-C₆alkyl-aryl, —C₁-C₆alkyl-heteroaryl, —C₁-C₆alkyl-heterocyclyl, or —C₁-C₆alkyl-cycloalkyl and R^{c} is —C₁-C₆alkyl, then R^{a} and R^{c} are optionally connected with a carbon atom to form a 5 to 10-membered heterocyclyl, heterocyclyl-aryl, heterocyclyl-heteroaryl, heterocyclyl-heterocyclyl or heterocyclyl-cycloalkyl, each of which is optionally substituted;
Z is selected from the group consisting of a covalent bond, —C₃-C₈alkyl-, —C₀-C₃alkyl-C₁-C₈heteroalkyl-C₀-C₃alkyl-, —C₀-C₃alkyl-C₂-C₈alkenyl-C₀-C₃alkyl-, —C₀-C₃alkyl-C₂-C₈alkynyl-C₀-C₃alkyl-, —C₀-C₆alkyl-aryl-C₀-C₆alkyl-, —C₀-C₆alkyl-aryl-C₂-C₆heteroalkyl-, —C₀-C₆alkyl-cycloalkyl-C₀-C₆alkyl-, —C₀-C₆alkyl-cycloalkyl-C₂-C₆heteroalkyl-, —C₄-C₆heterocyclyl-aryl-C₀-C₆alkyl-, —C₄-C₆heterocyclyl-aryl-C₀-C₆heteroalkyl-, —C₀-C₆alkyl-C₄-C₆-heterocyclyl-C₀-C₆alkyl-, —C₀-C₆alkyl-C₄-C₆-heterocyclyl-C₀-C₆heteroalkyl-, —C₀-C₆-alkyl-heteroaryl-C₀-C₆alkyl-, —C₀-C₆-alkyl-heteroaryl-C₀-C₆heteroalkyl-, —C₄-C₆heterocyclyl-heteroaryl-C₀-C₆alkyl-, —C₄-C₆heterocyclyl-heteroaryl-C₀-C₆heteroalkyl-, —C₀-C₆alkyl-aryl-C₃-C₆alkynyl-, —C₀-C₆alkyl-heteroaryl-C₂-C₆alkynyl-, —C₀-C₆alkyl-cycloalkyl-C₃-C₆alkynyl-, —C₀-C₆alkyl-heterocyclyl-C₃-C₆alkynyl-, —C₀-C₆alkyl-aryl-C₂-C₆alkynyl-C₂-C₆alkenyl-, —C₀-C₆alkyl-aryl-C₂-C₆alkenyl-, —C₀-C₆alkyl-heteroaryl-C₂-C₆alkenyl-, —C₀-C₆alkyl-cycloalkyl-C₂-C₆alkenyl-, —C₀-C₆alkyl-heterocyclyl-C₂-C₆alkenyl-, —C₀-C₆alkyl-aryl-aryl-C₀-C₆alkyl-, —C₀-C₆alkyl-aryl-heteroaryl-C₀-C₆alkyl-, —C₀-C₆alkyl-heteroaryl-aryl-C₀-C₆-alkyl-, —C₀-C₆alkyl-heteroaryl-heteroaryl-C₀-C₆-alkyl-, —C₀-C₃alkyl-heteroaryl-C₀-C₃alkyl-C₂-C₈ alkenyl-C₀-C₃-alkyl-, —CO—C₃alkyl-aryl-C₀-C₃alkyl-C₂-C₈ alkenyl-C₀-C₃-alkyl-, —C₀-C₃alkyl-heterocyclyl-C₀-C₃alkyl-C₂-C₈ alkenyl-C₀-C₃-alkyl-, —C₀-C₃alkyl-cycloalkyl-C₀-C₃alkyl-C₂-C₈ alkenyl-C₀-C₃-alkyl-, —C₀-C₃alkyl-heteroaryl-C₀-C₃alkyl-C₂-C₈ alkynyl-C₀-C₃-alkyl-, —C₀-C₃alkyl-aryl-C₀-C₃alkyl-C₂-C₈ alkynyl-C₀-C₃-alkyl-, —C₀-C₃alkyl-heterocyclyl-C₀-C₃alkyl-C₂-C₈ alkynyl-C₀-C₃-alkyl- and —C₀-C₃alkyl-cycloalkyl-C₀-C₃alkyl-C₂-C₈ alkynyl-C₀-C₃-alkyl-, wherein each alkyl, alkenyl, alkynyl, heteroalkyl, aryl, heteroaryl, heterocyclyl, and cycloalkyl moiety is optionally substituted; and
when W is N, Z is further selected from the group consisting of —C₁-C₈alkyl-C(O)—, —C₁-C₈ alkyl-S(O)₂—, -aryl-C₀-C₈ alkyl-S(O)₂—, -heteroaryl-C₀-C₈ alkyl-S(O)₂—, -cycloalkyl-C₀-C₈ alkyl-S(O)₂—, -heterocycloalkyl-C₀-C₈ alkyl-S(O)₂—, —C₁-C₈ alkyl-CH═, —C₁-C₈ alkyl-C(CH₃)═, —C₀-C₆alkyl-CH═CH—C(O), —C₀-C₄alkyl-C(O)—, —C₁-C₈ alkyl-(NH₂)C═, —C₀-C₃alkyl-C₁-C₈heteroalkyl-C₀-C₃alkyl-C(O)—, —C₀-C₃alkyl-C₂-C₈alkenyl-C₀-C₃alkyl-C(O)—, —C₀-C₃alkyl-C₂-C₅alkynyl-C₀-C₃alkyl-C(O)—, —C₀-C₆alkyl-aryl-C₀-C₆alkyl-C(O)—, —C₀-C₆alkyl-aryl-C₂-C₆heteroalkyl-C(O)—, —C₀-C₆alkyl-cycloalkyl-C₀-C₆alkyl-C(O)—, —C₀-C₆alkyl-cycloalkyl-C₂-C₆heteroalkyl-C(O)—, —C₄-C₆heterocyclyl-aryl-C₀-C₆alkyl-C(O)—, —C₄-C₆heterocyclyl-aryl-C₀-C₆heteroalkyl-C(O)—, —C₀-C₆alkyl-C₄-C₆-heterocyclyl-C₀-C₆alkyl-C(O)—, —C₀-C₆alkyl-C₄-C₆-heterocyclyl-C₀-C₆heteroalkyl-C(O)—, —C₀-C₆-alkyl-heteroaryl-C₀-C₆alkyl-C(O)—, —C₀-C₆-alkyl-heteroaryl-C₀-C₆heteroalkyl-C(O)—, —C₄-C₆heterocyclyl-heteroaryl-C₀-C₆alkyl-C(O)—, —C₄-C₆heterocyclyl-heteroaryl-C₀-C₆heteroalkyl-C(O)—, —C₀-C₆alkyl-aryl-C₃-C₆alkynyl-C(O)—, —C₀-C₆alkyl-heteroaryl-C₂-C₆alkynyl-C(O)—, —C₀-C₆alkyl-cycloalkyl-C₃-C₆alkynyl-C(O)—, —C₀-C₆alkyl-heterocyclyl-C₃-C₆alkynyl-C(O)—, —C₀-C₆alkyl-aryl-C₂-C₆alkynyl-C₂-C₆alkenyl-C(O)—, —C₀-C₆alkyl-aryl-C₂-C₆alkenyl-C(O)—, —C₀-C₆alkyl-heteroaryl-C₂-C₆alkenyl-C(O)—, —C₀-C₆alkyl-cycloalkyl-C₂-C₆alkenyl-C(O)—, —C₀-C₆alkyl-heterocyclyl-C₂-C₆alkenyl-C(O)—, —C₀-C₆alkyl-aryl-aryl-C₀-C₆alkyl-C(O)—, —C₀-C₆alkyl-aryl-heteroaryl-C₀-C₆alkyl-C(O)—, —C₀-C₆alkyl-heteroaryl-aryl-C₀-C₆-alkyl-C(O)— and —C₀-C₆alkyl-heteroaryl-heteroaryl-C₀-C₆-alkyl-C(O)—; or
Z-W is selected from the group consisting of —C₁-C₈ alkyl-(NH₂)C═N—, —C₁₋₈alkyl-C═N— and —C₁₋₈alkyl-C(CH₃)═N—, when R^{c} is absent;
L is selected from the group consisting of a covalent bond, —C₁-C₆alkyl-, —C₀-C₆alkyl-(CR³═CR³)₁₋₂—C₀-C₆alkyl-, —C₀-C₆alkyl-(C≡C)₁₋₂—C₀-C₆alkyl-, —C₀-C₆alkyl-aryl-C₀-C₆alkyl-, —C₀-C₆alkyl-heteroaryl-C₀-C₆alkyl-, —C₀-C₃alkyl-heterocyclyl-C₀-C₃alkyl-, —C₀-C₃alkyl-cycloalkyl-C₀-C₃alkyl-, —C₀-C₆alkyl-aryl-(CR³═CR³)₁₋₂—C₀-C₆alkyl-, —C₀-C₆ alkyl-aryl-(C≡C)₁₋₂—C₀-C₆alkyl-, —C₀-C₆alkyl-heteroaryl-(CR³═CR³)₁₋₂—C₀-C₆alkyl-, —C₀-C₆ alkyl-heteroaryl-(C≡C)₁₋₂—C₀-C₆alkyl-, —C₀-C₆alkyl-N(R³)—C(O)—C₀-C₃alkyl-, —C₀-C₆alkyl-N(R³)—C(S)—C₀-C₃alkyl-, —C₀-C₆alkyl-N(R³)—C(O)-alkenyl-C₀-C₃alkyl-, —C₀-C₆alkyl-N(R³)—C(S)-alkenyl-C₀-C₃alkyl-, —C₀-C₆alkyl-N(R³)—C(O)-alkynyl-C₀-C₃alkyl-, —C₀-C₆alkyl-N(R³)—C(S)-alkynyl-C₀-C₃alkyl-, —C₀-C₆alkyl-N(R³)—C(O)—C₁-C₃alkyl-N(R³)—C(O)—, —C₀-C₆alkyl-N(R³)—C(S)—C₁-C₃alkyl-N(R³)—C(O)—, —C₀-C₆alkyl-N(R³)—C(O)—C₁-C₃alkyl-N(R³)—C(O)—C₀-C₃alkyl-, —C₀-C₆alkyl-N(R³)—C(S)—C₁-C₃alkyl-N(R³)—C(O)—C₀-C₃alkyl-, —C₀-C₆alkyl-N(R³)—C(O)—C₁-C₃alkyl-N(R³)—C(S)—C₀-C₃alkyl-, —C₀-C₆alkyl-N(R³)—C(S)—C₁-C₃alkyl-N(R³)—C(S)—C₀-C₃alkyl-, —C₀-C₆alkyl-C(O)—N(R³)—C₀-C₃alkyl-, —C₀-C₆alkyl-C(S)—N(R³)—C₀-C₃alkyl-, —C₀-C₆alkyl-C(O)—N(R³)—C₂-C₄alkyl-O—C₀-C₃alkyl-, —C₀-C₆alkyl-C(S)—N(R³)—C₂-C₄alkyl-O—C₀-C₃alkyl-, —C₀-C₆alkyl-C(O)—N(R³)—C₂-C₄alkyl-N(R³)—C₀-C₃alkyl-, —C₀-C₆alkyl-C(S)—N(R³)—C₂-C₄alkyl-N(R³)—C₀-C₃alkyl-, —C₀-C₆alkyl-C(O)—C₀-C₃alkyl-, —C₀-C₆alkyl-C(NOH)—C₀-C₃alkyl-, —C₀-C₆alkyl-C(O)-alkenyl-C₀-C₃alkyl-, —C₀-C₆alkyl-C(NOH)-alkenyl-C₀-C₃alkyl-, —C₀-C₆alkyl-S(O)₂—N(R³)—C₀-C₃alkyl-, —C₀-C₆alkyl-N(R³)—S(O)₂—C₀-C₃alkyl-, —C₀-C₃alkyl-N(R³)—S(O)₂—N(R³)—C₀-C₃alkyl-, —C₀-C₆alkyl-C(O)—N(R³)—S(O)₂—C₀-C₃alkyl-, —C₀-C₆alkyl-N(R³)—C₀-C₃alkyl-, —C₀-C₆alkyl-N(R³)—C₁-C₃alkyl-alkenyl-, —C₀-C₆alkyl-N(R³)—C₁-C₃alkyl-alkynyl-, —C₀-C₆alkyl-N(R⁷)—C₀-C₃alkyl-, —C₀-C₆alkyl-N(R⁷)—C₁-C₃alkyl-alkenyl-, —C₀-C₆alkyl-N(R⁷)—C₁-C₃alkyl-alkynyl-, —C₀-C₆alkyl-N(R³)—C₂-C₄alkyl-N(R³)—C(O)-alkyl-, —C₀-C₆alkyl-N(R³)—C₂-C₄alkyl-N(R³)—C(S)-alkyl-, —C₀-C₆alkyl-O—C₂-C₄alkyl-N(R³)C(O)—C₀-C₃alkyl-, —C₀-C₆alkyl-S—C₂-C₄alkyl-N(R³)C(O)—C₀-C₃alkyl-, —C₀-C₆alkyl-O—C₂-C₄alkyl-N(R³)C(S)—C₀-C₃alkyl-, —C₀-C₆alkyl-S—C₂-C₄alkyl-N(R³)C(S)—C₀-C₃alkyl-, —C₀-C₆alkyl-N(R³)—C₂-C₃heteroalkyl-, —C₀-C₆alkyl-N(R⁷)—C₂-C₃heteroalkyl-, —C₀-C₆alkyl-C(O)—N(R³)—C₂-C₃heteroalkyl-, —C₀-C₆alkyl-C(S)—N(R³)—C₂-C₃heteroalkyl-, —C₀-C₆alkyl-C(O)—N(R⁷)—C₂-C₃heteroalkyl-, —C₀-C₆alkyl-C(S)—N(R⁷)—C₂-C₃heteroalkyl-, —C₀-C₆alkyl-N(R³)—C(O)—C₀-C₃heteroalkyl-, —C₀-C₆alkyl-N(R³)—C(S)—C₀-C₃heteroalkyl-, —C₀-C₆alkyl-N(R⁷)—C(O)—C₀-C₃heteroalkyl-, —C₀-C₆alkyl-N(R⁷)—C(S)—C₀-C₃heteroalkyl-, —C₀-C₆alkyl-S—C₀-C₃alkyl-, —C₀-C₆alkyl-O—C₀-C₃alkyl-, —C₀-C₆alkyl-S(O)—C₀-C₃alkyl-, —C₀-C₆alkyl-S(O)₂—C₀-C₃alkyl-, —C₀-C₆alkyl-N(R³)—C(O)—N(R³)—C₀-C₃alkyl-, —C₀-C₆alkyl-N(R³)—C(S)—N(R³)—C₀-C₃alkyl-, —C₀-C₆alkyl-O—C₀-C₃alkyl-heterocyclyl-, —C₀-C₆alkyl-O—C₀-C₃alkyl-cycloalkyl-, —C₀-C₆alkyl-S(O)₀₋₂—C₀-C₃alkyl-heterocycly-, —C₀-C₆alkyl-S(O)₀₋₂—C₀-C₃alkyl-cycloalkyl-, —C₀-C₆alkyl-N(R³)—C₀-C₃alkyl-heterocycly-, —C₀-C₆alkyl-N(R³)—C₀-C₃alkyl-cycloalkyl-, -heterocyclyl-C₀-C₆alkyl-O—C₀-C₃alkyl-, -cycloalkyl-C₀-C₆alkyl-O—C₀-C₃alkyl-, -heterocyclyl-C₀-C₆alkyl-S(O)₀₋₂—C₀-C₃alkyl-, -cycloalkyl-C₀-C₆alkyl-S(O)₀₋₂—C₀-C₃alkyl-, -heterocyclyl-C₀-C₆alkyl-N(R³)—C₀-C₃alkyl-, -cycloalkyl-C₀-C₆alkyl-N(R³)—C₀-C₃alkyl-, —C₀-C₆alkyl-N(R³)—C(O)—O—C₀-C₃alkyl-, —C₀-C₆alkyl-N(R³)—C(S)—O—C₀-C₃alkyl-, —C₀-C₆alkyl-O—C(O)—N(R³)—C₀-C₃alkyl-, —C₀-C₆alkyl-O—C(S)—N(R³)—C₀-C₃alkyl-, —C₀-C₃alkyl-N(R³)—C(O)—O-heterocyclyl-C₀-C₃alkyl-, —C₀-C₃alkyl-N(R³)—C(S)—O-heterocyclyl-C₀-C₃alkyl-, —C₀-C₃alkyl-N(R³)—C(O)—O-cycloalkyl-C₀-C₃alkyl-, —C₀-C₃alkyl-N(R³)—C(S)—O-cycloalkyl-C₀-C₃alkyl-, —C₀-C₆alkyl-S(O)₂-heterocyclyl-C₀-C₃alkyl-, —C₀-C₆alkyl-S(O)₂-cycloalkyl-C₀-C₃alkyl-, —C₀-C₆ alkyl-N(R³)—S(O)₂-heterocyclyl-C₀-C₃alkyl-, —C₀-C₆ alkyl-N(R³)—S(O)₂-cycloalkyl-C₀-C₃alkyl-, —C₀-C₃alkyl-heterocyclyl-C₀-C₃alkyl-O—C₀-C₃alkyl-, —C₀-C₃alkyl-cycloalkyl-C₀-C₃alkyl-O—C₀-C₃alkyl-, —C₀-C₃alkyl-heterocyclyl-C₀-C₅alkyl-N(R³)—C₀-C₃alkyl-, —C₀-C₃alkyl-cycloalkyl-C₀-C₅alkyl-N(R³)—C₀-C₃alkyl-, —C₀-C₃alkyl-O—C₀-C₃alkyl-heterocyclyl-C₀-C₃alkyl-, —C₀-C₃alkyl-O—C₀-C₃alkyl-cycloalkyl-C₀-C₃alkyl-, —C₀-C₃alkyl-heterocyclyl-C₀-C₃alkyl-S—C₀-C₃alkyl-, —C₀-C₃alkyl-cycloalkyl-C₀-C₃alkyl-S—C₀-C₃alkyl-, —C₀-C₃alkyl-N(R³)—C₀-C₃alkyl-heterocyclyl-C₀-C₃alkyl-, —C₀-C₃alkyl-N(R³)—C₀-C₃alkyl-cycloalkyl-C₀-C₃alkyl-, —C₀-C₃alkyl-S—C₀-C₃alkyl-heterocyclyl-C₀-C₃alkyl-, —C₀-C₃alkyl-S—C₀-C₃alkyl-cycloalkyl-C₀-C₃alkyl-, —C₀-C₃alkyl-S(O)₂N(R³)—C₀-C₃alkyl-heterocyclyl-C₀-C₃alkyl-, —C₀-C₃alkyl-S(O)₂N(R³)—C₀-C₃alkyl-cycloalkyl-C₀-C₃alkyl-, —C₀-C₃alkyl-C(O)—N(R³)—C₀-C₃alkyl-heterocyclyl-C₀-C₃alkyl-, —C₀-C₃alkyl-C(O)—N(R³)—C₀-C₃alkyl-cycloalkyl-C₀-C₃alkyl-, —C₀-C₃alkyl-C(S)—N(R³)—C₀-C₃alkyl-heterocyclyl-C₀-C₃alkyl-, —C₀-C₃alkyl-C(S)—N(R³)—C₀-C₃alkyl-cycloalkyl-C₀-C₃alkyl-, —C₀-C₃alkyl-C(O)-heterocyclyl-C₀-C₃alkyl-, —C₀-C₃alkyl-C(O)-cycloalkyl-C₀-C₃alkyl-, —C₀-C₃alkyl-O—C(O)-heterocyclyl-C₀-C₃alkyl-, —C₀-C₃alkyl-O—C(O)-cycloalkyl-C₀-C₃alkyl-, —C₀-C₃alkyl-O—C(O)—N(R³)—C₀-C₃alkyl-heterocyclyl-C₀-C₃alkyl-, —C₀-C₃alkyl-O—C(O)—N(R³)—C₀-C₃alkyl-cycloalkyl-C₀-C₃alkyl-, —C₀-C₃alkyl-O—C(S)—N(R³)—C₀-C₃alkyl-heterocyclyl-C₀-C₃alkyl-, —C₀-C₃alkyl-O—C(S)—N(R³)—C₀-C₃alkyl-cycloalkyl-C₀-C₃alkyl-, —C₀-C₃alkyl-N(R³)—C(O)—N(R³)—C₀-C₃alkyl-heterocyclyl-C₀-C₃alkyl-, —C₀-C₃alkyl-N(R³)—C(O)—N(R³)—C₀-C₃alkyl-cycloalkyl-C₀-C₃alkyl-, —C₀-C₃alkyl-N(R³)—C(S)—N(R³)—C₀-C₃alkyl-heterocyclyl-C₀-C₃alkyl-, —C₀-C₃alkyl-N(R³)—C(S)—N(R³)—C₀-C₃alkyl-cycloalkyl-C₀-C₃alkyl-, —C₀-C₃alkyl-N(R³)—C(O)-heterocyclyl-C₀-C₃alkyl-, —C₀-C₃alkyl-N(R³)—C(O)-cycloalkyl-C₀-C₃alkyl-, —C₀-C₃alkyl-N(R³)—C(S)-heterocyclyl-C₀-C₃alkyl-, —C₀-C₃alkyl-N(R³)—C(S)-cycloalkyl-C₀-C₃alkyl-, —C₀-C₃alkyl-N(R³)—S(O)₂—N(R³)—C₀-C₃alkyl-heterocyclyl-C₀-C₃alkyl-, —C₀-C₃alkyl-N(R³)—S(O)₂—N(R³)—C₀-C₃alkyl-cycloalkyl-C₀-C₃alkyl-, —C₀-C₆alkyl-heterocyclyl-C(O)—C₀-C₆alkyl-, —C₀-C₆ alkyl-cycloalkyl-C(O)—C₀-C₆alkyl-, —C₀-C₆alkyl-N(R³)—C(O)-heterocyclyl-C(O)—C₀-C₆alkyl-, —C₀-C₆alkyl-N(R³)—C(O)-cycloalkyl-C(O)—C₀-C₆alkyl-, —C₀-C₆alkyl-N(R³)—C(S)-heterocyclyl-C(O)—C₀-C₆alkyl-, —C₀-C₆alkyl-N(R³)—C(S)-cycloalkyl-C(O)—C₀-C₆alkyl-, —C₀-C₆alkyl-heterocyclyl-S(O)₂—C₀-C₆alkyl-, —C₀-C₆alkyl-cycloalkyl-S(O)₂—C₀-C₆alkyl-, —C₀-C₆alkyl-heterocyclyl-N(R³)—C(O)—C₀-C₆alkyl-, —CO—C₆alkyl-cycloalkyl-N(R³)—C(O)—C₀-C₆alkyl-, —C₀-C₆alkyl-heterocyclyl-O—C(O)—C₀-C₆alkyl-, —C₀-C₆alkyl-cycloalkyl-O—C(O)—C₀-C₆alkyl-, —C₀-C₆alkyl-heterocyclyl-N(R³)—C(S)—C₀-C₆alkyl-, —C₀-C₆alkyl-cycloalkyl-N(R³)—C(S)—C₀-C₆alkyl-, —C₀-C₆alkyl-heterocyclyl-O—C(S)—C₀-C₆alkyl-, —C₀-C₆alkyl-cycloalkyl-O—C(S)—C₀-C₆alkyl-, —C₀-C₃alkyl-CH═N—O—C₀-C₃alkyl-, —C₀-C₃alkyl-C═N(OH)C(O)—N(R³)—C₂-C₄-alkyl-S—S-alkyl-, —C₀-C₆alkyl-heteroalkyl-C₀-C₆alkyl-C(O)—N(R³)—C₀-C₃alkyl-, —C₀-C₆alkyl-heteroalkyl-C₀-C₆alkyl-C(S)—N(R³)—C₀-C₃alkyl-, —C₀-C₆alkyl-aryl-heteroaryl-C₀-C₆alkyl-, —C₀-C₆alkyl-heteroaryl-aryl-C₀-C₆alkyl-, —C₀-C₆alkyl-heteroaryl-heteroaryl-C₀-C₆alkyl-, —C₀-C₆alkyl-aryl-aryl-C₀-C₆alkyl-, —C₁-C₃alkyl-N(R³)—C(O)—C₁-C₇alkyl-, —C₁-C₃alkyl-N(R³)—C(S)—C₁-C₇alkyl-, —C₀-C₃alkyl-alkenyl-C(O)—C₀-C₆alkyl-, —C₀-C₆alkyl-N(R³)—C(O)—N(R³)—S(O)₀₋₂—C₀-C₃alkyl-, —C₀-C₃alkyl-O-aryl-C₀-C₆ alkyl-N(R³)—C(O)—C₁-C₃ alkyl-, —C₀-C₃alkyl-S-aryl-C₀-C₆ alkyl-N(R³)C(O)—C₁-C₃ alkyl-, —C₀-C₃alkyl-O-aryl-C₀-C₆ alkyl-N(R³)C(S)—C₁-C₃ alkyl-, —C₀-C₃alkyl-S-aryl-C₀-C₆ alkyl-N(R³)C(S)—C₁-C₃ alkyl-, —C₀-C₃alkyl-O-heteroaryl-C₀-C₆ alkyl-N(R³)—C(O)—C₁-C₃alkyl-, —C₀-C₃alkyl-S-heteroaryl-C₀-C₆ alkyl-N(R³)—C(O)—C₁-C₃alkyl-, —C₀-C₃alkyl-O-heteroaryl-C₀-C₆ alkyl-N(R³)—C(S)—C₁-C₃alkyl-, —C₀-C₃alkyl-S-heteroaryl-C₀-C₆ alkyl-N(R³)—C(S)—C₁-C₃alkyl-, —C₀-C₃alkyl-O-heterocyclyl-C₀-C₆ alkyl-N(R³)—C(O)—C₁-C₃alkyl-, —C₀-C₃alkyl-S-heterocyclyl-C₀-C₆ alkyl-(NR³)—C(O)—C₁-C₃alkyl-, —C₀-C₃alkyl-O-heterocyclyl-C₀-C₆ alkyl-N(R³)—C(S)—C₁-C₃alkyl-, —C₀-C₃alkyl-S— heterocyclyl-C₀-C₆ alkyl-N(R³)—C(S)—C₁-C₃alkyl-, —C₀-C₃alkyl-O-cycloalkyl-C₀-C₆alkyl-N(R³)—C(O)—C₁-C₃alkyl-, —C₀-C₃alkyl-S-cycloalkyl-C₀-C₆alkyl-N(R³)— C(O)—C₁-C₃alkyl-, —C₀-C₃alkyl-O-cycloalkyl-C₀-C₆alkyl-N(R³)—C(S)—C₁-C₃alkyl-, —C₀-C₃alkyl-S-cycloalkyl-C₀-C₆alkyl-N(R³)—C(S)—C₁-C₃alkyl-, —C₀-C₃alkyl-N(R³)-aryl-C₀-C₆alkyl-N(R³)— C(O)—C₁-C₃alkyl-, —C₀-C₃alkyl-N(R³)-aryl-C₀-C₆alkyl-N(R³)—C(S)—C₁-C₃alkyl-, —C₀-C₃alkyl-N(R³)-heteroaryl-C₀-C₆alkyl-N(R³)—C(O)—C₁-C₃alkyl-, —C₀-C₃alkyl-N(R³)-heteroaryl-C₀-C₆alkyl-N(R³)—C(S)—C₁-C₃alkyl-, —C₀-C₃alkyl-N(R³)-heterocyclyl-C₀-C₆alkyl-N(R³)— C(O)—C₁-C₃ alkyl-, —C₀-C₃alkyl-N(R³)-heterocyclyl-C₀-C₆alkyl-N(R³)—C(S)—C₁-C₃ alkyl-, —C₀-C₃alkyl-N(R³)-cycloalkyl-C₀-C₆alkyl-N(R³)—C(O)—C₁-C₃alkyl-, —C₀-C₃alkyl-N(R³)-cycloalkyl-C₀-C₆alkyl-N(R³)—C(S)—C₁-C₃alkyl-, —C₀-C₃alkyl-N(R³)—C(O)—O-aryl-C₀-C₆alkyl-N(R³)— C(O)—C₁-C₃alkyl-, —C₀-C₃alkyl-N(R³)—C(S)—O-aryl-C₀-C₆alkyl-N(R³)—C(O)—C₁-C₃alkyl-, —C₀-C₃alkyl-N(R³)—C(O)—S-aryl-C₀-C₆alkyl-N(R³)—C(O)—C₁-C₃alkyl-, —C₀-C₃alkyl-N(R³)—C(O)—O-aryl-C₀-C₆alkyl-N(R³)—C(S)—C₁-C₃alkyl-, —C₀-C₃alkyl-N(R³)—C(S)—O-aryl-C₀-C₆alkyl-N(R³)—C(S)—C₁-C₃alkyl-, —C₀-C₃alkyl-N(R³)—C(O)—S-aryl-C₀-C₆alkyl-N(R³)—C(S)—C₁-C₃alkyl-, —C₀-C₃alkyl-N(R³)—C(O)—O-heteroaryl-C₀-C₆alkyl-N(R³)—C(O)—C₁-C₃alkyl-, —C₀-C₃alkyl-N(R³)—C(S)—O-heteroaryl-C₀-C₆alkyl-N(R³)—C(O)—C₁-C₃alkyl-, —C₀-C₃alkyl-N(R³)—C(O)—S-heteroaryl-C₀-C₆alkyl-N(R³)—C(O)—C₁-C₃alkyl-, —C₀-C₃alkyl-N(R³)—C(O)—O-heteroaryl-C₀-C₆alkyl-N(R³)—C(S)—C₁-C₃alkyl-, —C₀-C₃alkyl-N(R³)—C(S)—O-heteroaryl-C₀-C₆alkyl-N(R³)—C(S)—C₁-C₃alkyl-, —C₀-C₃alkyl-N(R³)—C(O)—O-heterocyclyl-C₀-C₆alkyl-N(R³)—C(O)—C₁-C₃alkyl-, —C₀-C₃alkyl-N(R³)—C(S)—O-heterocyclyl-C₀-C₆alkyl-N(R³)—C(O)—C₁-C₃alkyl-, —C₀-C₃alkyl-N(R³)—C(O)—O-heterocyclyl-C₀-C₆alkyl-N(R³)—C(S)—C₁-C₃alkyl-, —C₀-C₃alkyl-N(R³)—C(S)—O-heterocyclyl-C₀-C₆alkyl-N(R³)—C(S)—C₁-C₃alkyl-, —C₀-C₃alkyl-N(R³)—C(O)—O-cycloalkyl-C₀-C₆alkyl-N(R³)—C(O)—C₁-C₃alkyl-, —C₀-C₃alkyl-N(R³)—C(S)—O-cycloalkyl-C₀-C₆alkyl-N(R³)—C(O)—C₁-C₃alkyl-, —C₀-C₃alkyl-N(R³)—C(O)—O-cycloalkyl-C₀-C₆alkyl-N(R³)—C(S)—C₁-C₃alkyl-, —C₀-C₃alkyl-N(R³)—C(S)—O-cycloalkyl-C₀-C₆alkyl-N(R³)—C(S)—C₁-C₃alkyl-, —C₀-C₃alkyl-C(O)—N(R³)-aryl-C₀-C₆alkyl-N(R³)—C(O)—C₁-C₃alkyl-, —CO—C₃alkyl-C(S)—N(R³)-aryl-C₀-C₆alkyl-N(R³)—C(O)—C₁-C₃alkyl-, —C₀-C₃alkyl-C(O)—N(R³)-aryl-C₀-C₆alkyl-N(R³)—C(S)—C₁-C₃alkyl-, —C₀-C₃alkyl-C(S)—N(R³)-aryl-C₀-C₆alkyl-N(R³)—C(S)—C₁-C₃alkyl-, —C₀-C₃alkyl-C(O)—N(R³)-heteroaryl-C₀-C₆alkyl-N(R³)C(O)—C₁-C₃alkyl-, —C₀-C₃alkyl-C(S)—N(R³)-heteroaryl-C₀-C₆alkyl-N(R³)C(O)—C₁-C₃alkyl-, —C₀-C₃alkyl-C(O)—N(R³)-heteroaryl-C₀-C₆alkyl-N(R³)C(S)—C₁-C₃alkyl-, —C₀-C₃alkyl-C(S)—N(R³)-heteroaryl-C₀-C₆alkyl-N(R³)C(S)—C₁-C₃alkyl-, —C₀-C₃alkyl-C(O)—N(R³)-heterocyclyl-C₀-C₆alkyl-N(R³)—C(O)—C₁-C₃alkyl-, —C₀-C₃alkyl-C(S)—N(R³)-heterocyclyl-C₀-C₆alkyl-N(R³)—C(O)—C₁-C₃alkyl-, —C₀-C₃alkyl-C(O)—N(R³)-heterocyclyl-C₀-C₆alkyl-N(R³)—C(S)—C₁-C₃alkyl-, —C₀-C₃alkyl-C(S)—N(R³)-heterocyclyl-C₀-C₆alkyl-N(R³)—C(S)—C₁-C₃alkyl-, —C₀-C₃alkyl-C(O)—N(R³)-cycloalkyl-C₀-C₆alkyl-N(R³)—C(O)—C₁-C₃alkyl-, —C₀-C₃alkyl-C(S)—N(R³)-cycloalkyl-C₀-C₆alkyl-N(R³)—C(O)—C₁-C₃alkyl-, —C₀-C₃alkyl-C(O)—N(R³)— cycloalkyl-C₀-C₆alkyl-N(R³)—C(S)—C₁-C₃alkyl-, —C₀-C₃alkyl-C(S)—N(R³)-cycloalkyl-C₀-C₆alkyl-N(R³)—C(S)—C₁-C₃alkyl-, —C₀-C₃alkyl-O—C(O)—N(R³)-aryl-C₀-C₆alkyl-N(R³)—C(O)—C₁-C₃alkyl-, —C₀-C₃alkyl-O—C(S)—N(R³)-aryl-C₀-C₆alkyl-N(R³)— C(O)—C₁-C₃alkyl-, —C₀-C₃alkyl-O—C(O)—N(R³)— aryl-C₀-C₆alkyl-N(R³)—C(S)—C₁-C₃alkyl-, —C₀-C₃alkyl-O—C(S)—N(R³)-aryl-C₀-C₆alkyl-N(R³)—C(S)—C₁-C₃alkyl-, —C₀-C₃alkyl-O—C(O)—N(R³)-heteroaryl-C₀-C₆alkyl-N(R³)—C(O)—C₁-C₃alkyl-, —C₀-C₃alkyl-O—C(S)—N(R³)-heteroaryl-C₀-C₆alkyl-N(R³)—C(O)—C₁-C₃alkyl-, —C₀-C₃alkyl-O—C(O)—N(R³)-heteroaryl-C₀-C₆alkyl-N(R³)—C(S)—C₁-C₃alkyl-, —C₀-C₃alkyl-O—C(S)—N(R³)-heteroaryl-C₀-C₆alkyl-N(R³)—C(S)—C₁-C₃alkyl-, —C₀-C₃alkyl-O—C(O)—N(R³)-heterocyclyl-C₀-C₆alkyl-N(R³)—C(O)—C₁-C₃alkyl-, —C₀-C₃alkyl-O—C(S)—N(R³)-heterocyclyl-C₀-C₆alkyl-N(R³)—C(O)—C₁-C₃alkyl-, —C₀-C₃alkyl-O—C(O)—N(R³)-heterocylcyl-C₀-C₆alkyl-N(R³)—C(S)—C₁-C₃alkyl-, —C₀-C₃alkyl-O—C(S)—N(R³)-heterocyclyl-C₀-C₆alkyl-N(R³)—C(S)—C₁-C₃alkyl-, —C₀-C₃alkyl-O—C(O)—N(R³)-cycloalkyl-C₀-C₆alkyl-N(R³)—C(O)—C₁-C₃alkyl-, —C₀-C₃alkyl-O—C(S)—N(R³)-cycloalkyl-C₀-C₆alkyl-N(R³)—C(O)—C₁-C₃alkyl-, —C₀-C₃alkyl-O—C(O)—N(R³)-cycloalkyl-C₀-C₆alkyl-N(R³)—C(S)—C₁-C₃alkyl-, —C₀-C₃alkyl-O—C(S)—N(R³)-cycloalkyl-C₀-C₆alkyl-N(R³)—C(S)—C₁-C₃alkyl-, —C₀-C₃alkyl-S(O)₂—N(R³)-aryl-C₀-C₆alkyl-N(R³)—C(O)—C₁-C₃alkyl-, —C₀-C₃alkyl-S(O)₂—N(R³)-aryl-C₀-C₆alkyl-N(R³)—C(S)—C₁-C₃alkyl-, —C₀-C₃ alkyl-S(O)₂—N(R³)-heteroaryl-C₀-C₆alkyl-N(R³)—C(O)—C₁-C₃alkyl-, —C₀-C₃ alkyl-S(O)₂—N(R³)-heteroaryl-C₀-C₆alkyl-N(R³)—C(S)—C₁-C₃alkyl-, —C₀-C₃alkyl-S(O)₂—N(R³)-heterocyclyl-C₀-C₆alkyl-N(R³)—C(O)—C₁-C₃alkyl-, —C₀-C₃alkyl-S(O)₂—N(R³)-heterocyclyl-C₀-C₆alkyl-N(R³)—C(S)—C₁-C₃alkyl-, —C₀-C₃alkyl-S(O)₂—N(R³)-cycloalkyl-C₀-C₆alkyl-N(R³)—C(O)—C₁-C₃alkyl-, —C₀-C₃alkyl-S(O)₂—N(R³)-cycloalkyl-C₀-C₆alkyl-N(R³)—C(S)—C₁-C₃alkyl-, —C₀-C₃alkyl-N(R³)—S(O)₂—N(R³)-aryl-C₀-C₆alkyl-N(R³)—C(O)—C₁-C₃alkyl-, —C₀-C₃alkyl-N(R³)—S(O)₂—N(R³)-aryl-C₀-C₆alkyl-N(R³)—C(S)—C₁-C₃alkyl-, —C₀-C₃alkyl-N(R³)—S(O)₂—N(R³)-heteroaryl-C₀-C₆alkyl-N(R³)—C(O)—C₁-C₃alkyl-, —C₀-C₃alkyl-N(R³)—S(O)₂—N(R³)-heteroaryl-C₀-C₆alkyl-N(R³)—C(S)—C₁-C₃alkyl-, —C₀-C₃alkyl-N(R³)—S(O)₂—N(R³)-heterocyclyl-C₀-C₆alkyl-N(R³)—C(O)—C₁-C₃alkyl-, —C₀-C₃alkyl-N(R³)—S(O)₂—N(R³)-heterocyclyl-C₀-C₆alkyl-N(R³)—C(S)—C₁-C₃alkyl-, —C₀-C₃alkyl-N(R³)— S(O)₂—N(R³)-cycloalkyl-C₀-C₆alkyl-N(R³)—C(O)—C₁-C₃alkyl-, —C₀-C₃alkyl-N(R³)— S(O)₂—N(R³)-cycloalkyl-C₀-C₆alkyl-N(R³)—C(S)—C₁-C₃alkyl-, —C₀-C₃alkyl-heterocyclyl-O-aryl-C₀-C₆alkyl-N(R³)—C(O)—C₁-C₃alkyl-, —C₀-C₃alkyl-heterocyclyl-S-aryl-C₀-C₆alkyl-N(R³)—C(O)—C₁-C₃alkyl-, —CO—C₃alkyl-heterocyclyl-O-aryl-C₀-C₆alkyl-N(R³)—C(S)—C₁-C₃alkyl-, —C₀-C₃alkyl-heterocyclyl-S-aryl-C₀-C₆alkyl-N(R³)—C(S)—C₁-C₃alkyl-, —C₀-C₃alkyl-heterocyclyl-O-heteroaryl-C₀-C₆alkyl-N(R³)— C(O)—C₁-C₃alkyl-, —C₀-C₃alkyl-heterocyclyl-S-heteroaryl-C₀-C₆alkyl-N(R³)—C(O)—C₁-C₃alkyl-, —C₀-C₃alkyl-heterocyclyl-O-heteroaryl-C₀-C₆alkyl-N(R³)—C(S)—C₁-C₃alkyl-, —CO—C₃alkyl-heterocyclyl-S-heteroaryl-C₀-C₆alkyl-N(R³)—C(S)—C₁-C₃alkyl-, —C₀-C₃alkyl-heterocyclyl-O-heterocyclyl-C₀-C₆alkyl-N(R³)—C(O)—C₁-C₃alkyl-, —C₀-C₃alkyl-heterocyclyl-S-heterocyclyl-C₀-C₆alkyl-N(R³)—C(O)—C₁-C₃alkyl-, —C₀-C₃alkyl-heterocyclyl-O-heterocyclyl-C₀-C₆alkyl-N(R³)—C(S)—C₁-C₃alkyl-, —C₀-C₃alkyl-heterocyclyl-S-heterocyclyl-C₀-C₆alkyl-N(R³)—C(S)—C₁-C₃alkyl-, —C₀-C₃alkyl-heterocyclyl-O-cycloalkyl-C₀-C₆alkyl-N(R³)—C(O)—C₁-C₃alkyl-, —C₀-C₃alkyl-heterocyclyl-S-cycloalkyl-C₀-C₆alkyl-N(R³)—C(O)—C₁-C₃alkyl-, —C₀-C₃alkyl-heterocyclyl-O-cycloalkyl-C₀-C₆alkyl-N(R³)—C(S)—C₁-C₃alkyl-, —C₀-C₃alkyl-heterocyclyl-S-cycloalkyl-C₀-C₆alkyl-N(R³)—C(S)—C₁-C₃alkyl-, —C₀-C₃alkyl-C(O)-heterocyclyl-O-aryl-C₀-C₆alkyl-N(R³)—C(O)—C₁-C₃alkyl-, —C₀-C₃alkyl-C(S)-heterocyclyl-O-aryl-C₀-C₆alkyl-N(R³)—C(O)—C₁-C₃alkyl-, —C₀-C₃alkyl-C(O)-heterocyclyl-S-aryl-C₀-C₆alkyl-N(R³)— C(O)—C₁-C₃alkyl-, —C₀-C₃alkyl-C(O)-heterocyclyl-O-aryl-C₀-C₆alkyl-N(R³)—C(S)—C₁-C₃alkyl-, —C₀-C₃alkyl-C(S)-heterocyclyl-S-aryl-C₀-C₆alkyl-N(R³)—C(O)—C₁-C₃alkyl-, —C₀-C₃alkyl-C(S)-heterocyclyl-O-aryl-C₀-C₆alkyl-N(R³)—C(S)—C₁-C₃alkyl-, —C₀-C₃alkyl-C(O)-heterocyclyl-S-aryl-C₀-C₆alkyl-N(R³)—C(S)—C₁-C₃alkyl-, —C₀-C₃alkyl-C(S)-heterocyclyl-S-aryl-C₀-C₆alkyl-N(R³)—C(S)—C₁-C₃alkyl-, —C₀-C₃alkyl-C(O)-heterocyclyl-O-heteroaryl-C₀-C₆alkyl-N(R³)—C(O)—C₁-C₃alkyl-, —C₀-C₃alkyl-C(S)-heterocyclyl-O-heteroaryl-C₀-C₆alkyl-N(R³)—C(O)—C₁-C₃alkyl-, —C₀-C₃alkyl-C(O)-heterocyclyl-S-heteroaryl-C₀-C₆alkyl-N(R³)—C(O)—C₁-C₃alkyl-, —C₀-C₃alkyl-C(O)-heterocyclyl-O-heteroaryl-C₀-C₆alkyl-N(R³)—C(S)—C₁-C₃alkyl-, —C₀-C₃alkyl-C(S)-heterocyclyl-S-heteroaryl-C₀-C₆alkyl-N(R³)—C(O)—C₁-C₃alkyl-, —C₀-C₃alkyl-C(S)-heterocyclyl-O-heteroaryl-C₀-C₆alkyl-N(R³)—C(S)—C₁-C₃alkyl-, —C₀-C₃alkyl-C(O)-heterocyclyl-S-heteroaryl-C₀-C₆alkyl-N(R³)—C(S)—C₁-C₃alkyl-, —C₀-C₃alkyl-C(S)-heterocyclyl-S-heteroaryl-C₀-C₆alkyl-N(R³)—C(S)—C₁-C₃alkyl-, —C₀-C₃alkyl-C(O)-heterocyclyl-O-heterocyclyl-C₀-C₆alkyl-N(R³)—C(O)—C₁-C₃alkyl-, —C₀-C₃alkyl-C(S)-heterocyclyl-O-heterocyclyl-C₀-C₆alkyl-N(R³)—C(O)—C₁-C₃alkyl-, —C₀-C₃alkyl-C(O)-heterocyclyl-S-heterocyclyl-C₀-C₆alkyl-N(R³)—C(O)—C₁-C₃alkyl-, —C₀-C₃alkyl-C(O)-heterocyclyl-O-heterocyclyl-C₀-C₆alkyl-N(R³)—C(S)—C₁-C₃alkyl-, —C₀-C₃alkyl-C(S)-heterocyclyl-S-heterocyclyl-C₀-C₆alkyl-N(R³)—C(O)—C₁-C₃alkyl-, —C₀-C₃alkyl-C(S)-heterocyclyl-O-heterocyclyl-C₀-C₆alkyl-N(R³)—C(S)—C₁-C₃alkyl-, —C₀-C₃alkyl-C(O)-heterocyclyl-S-heterocyclyl-C₀-C₆alkyl-N(R³)—C(S)—C₁-C₃alkyl-, —C₀-C₃alkyl-C(S)-heterocyclyl-S-heterocyclyl-C₀-C₆alkyl-N(R³)—C(S)—C₁-C₃alkyl-, —C₀-C₃alkyl-C(O)-heterocyclyl-O-cycloalkyl-C₀-C₆alkyl-N(R³)—C(O)—C₁-C₃alkyl-, —C₀-C₃alkyl-C(S)-heterocyclyl-O-cycloalkyl-C₀-C₆alkyl-N(R³)—C(O)—C₁-C₃alkyl-, —C₀-C₃alkyl-C(O)-heterocyclyl-S-cycloalkyl-C₀-C₆alkyl-N(R³)—C(O)—C₁-C₃alkyl-, —C₀-C₃alkyl-C(O)-heterocyclyl-O-cycloalkyl-C₀-C₆alkyl-N(R³)—C(S)—C₁-C₃alkyl-, —C₀-C₃alkyl-C(S)-heterocyclyl-S-cycloalkyl-C₀-C₆alkyl-N(R³)—C(O)—C₁-C₃alkyl-, —C₀-C₃alkyl-C(S)-heterocyclyl-O-cycloalkyl-C₀-C₆alkyl-N(R³)—C(S)—C₁-C₃alkyl-, —C₀-C₃alkyl-C(O)-heterocyclyl-S-cycloalkyl-C₀-C₆alkyl-N(R³)—C(S)—C₁-C₃alkyl-, —C₀-C₃alkyl-C(S)-heterocyclyl-S-cycloalkyl-C₀-C₆alkyl-N(R³)—C(S)—C₁-C₃alkyl-, —C₀-C₃alkyl-N(R³)—C(O)-heterocyclyl-O-aryl-C₀-C₆alkyl-N(R³)—C(O)—C₁-C₃alkyl-, —C₀-C₃alkyl-N(R³)—C(S)-heterocyclyl-O-aryl-C₀-C₆alkyl-N(R³)—C(O)—C₁-C₃alkyl-, —C₀-C₃alkyl-N(R³)—C(O)-heterocyclyl-S-aryl-C₀-C₆alkyl-N(R³)—C(O)—C₁-C₃alkyl-, —C₀-C₃alkyl-N(R³)—C(O)-heterocyclyl-O-aryl-C₀-C₆alkyl-N(R³)—C(S)—C₁-C₃alkyl-, —C₀-C₃alkyl-N(R³)—C(S)-heterocyclyl-S-aryl-C₀-C₆alkyl-N(R³)—C(O)—C₁-C₃alkyl-, —C₀-C₃alkyl-N(R³)—C(S)-heterocyclyl-O-aryl-C₀-C₆alkyl-N(R³)—C(S)—C₁-C₃alkyl-, —C₀-C₃alkyl-N(R³)—C(O)-heterocyclyl-S-aryl-C₀-C₆alkyl-N(R³)—C(S)—C₁-C₃alkyl-, —C₀-C₃alkyl-N(R³)—C(S)-heterocyclyl-S-aryl-C₀-C₆alkyl-N(R³)—C(S)—C₁-C₃alkyl-, —C₀-C₃alkyl-N(R³)—C(O)-heterocyclyl—O-heteroaryl-C₀-C₆alkyl-N(R³)—C(O)—C₁-C₃alkyl-, —C₀-C₃alkyl-N(R³)—C(S)-heterocyclyl—O-heteroaryl-C₀-C₆alkyl-N(R³)—C(O)—C₁-C₃alkyl-, —C₀-C₃alkyl-N(R³)—C(O)-heterocyclyl-S-heteroaryl-C₀-C₆alkyl-N(R³)—C(O)—C₁-C₃alkyl-, —C₀-C₃alkyl-N(R³)—C(O)-heterocyclyl-O-heteroaryl-C₀-C₆alkyl-N(R³)—C(S)—C₁-C₃alkyl-, —C₀-C₃alkyl-N(R³)—C(S)-heterocyclyl-S-heteroaryl-C₀-C₆alkyl-N(R³)—C(O)—C₁-C₃alkyl-, —C₀-C₃alkyl-N(R³)—C(S)-heterocyclyl-O-heteroaryl-C₀-C₆alkyl-N(R³)—C(S)—C₁-C₃alkyl-, —C₀-C₃alkyl-N(R³)—C(O)-heterocyclyl-S-heteroaryl-C₀-C₆alkyl-N(R³)—C(S)—C₁-C₃alkyl-, —C₀-C₃alkyl-N(R³)—C(S)-heterocyclyl-S-heteroaryl-C₀-C₆alkyl-N(R³)—C(S)—C₁-C₃alkyl-, —C₀-C₃alkyl-N(R³)—C(O)-heterocyclyl-O-heterocyclyl-C₀-C₆alkyl-N(R³)—C(O)—C₁-C₃alkyl-, —C₀-C₃alkyl-N(R³)—C(S)-heterocyclyl-O-heterocyclyl-C₀-C₆alkyl-N(R³)—C(O)—C₁-C₃alkyl-, —C₀-C₃alkyl-N(R³)—C(O)-heterocyclyl-S-heterocyclyl-C₀-C₆alkyl-N(R³)—C(O)—C₁-C₃alkyl-, —C₀-C₃alkyl-N(R³)—C(O)-heterocyclyl-O-heterocyclyl-C₀-C₆alkyl-N(R³)—C(S)—C₁-C₃alkyl-, —C₀-C₃alkyl-N(R³)—C(S)-heterocyclyl-S-heterocyclyl-C₀-C₆alkyl-N(R³)—C(O)—C₁-C₃alkyl-, —C₀-C₃alkyl-N(R³)—C(S)-heterocyclyl-O-heterocyclyl-C₀-C₆alkyl-N(R³)—C(S)—C₁-C₃alkyl-, —C₀-C₃alkyl-N(R³)—C(O)-heterocyclyl-S-heterocyclyl-C₀-C₆alkyl-N(R³)—C(S)—C₁-C₃alkyl-, —C₀-C₃alkyl-N(R³)—C(S)-heterocyclyl-S-heterocyclyl-C₀-C₆alkyl-N(R³)—C(S)—C₁-C₃alkyl-, —C₀-C₃alkyl-N(R³)—C(O)-heterocyclyl-O-cycloalkyl-C₀-C₆alkyl-N(R³)—C(O)—C₁-C₃alkyl-, —C₀-C₃alkyl-N(R³)—C(S)-heterocyclyl-O-cycloalkyl-C₀-C₆alkyl-N(R³)—C(O)—C₁-C₃alkyl-, —C₀-C₃alkyl-N(R³)—C(O)-heterocyclyl-S-cycloalkyl-C₀-C₆alkyl-N(R³)—C(O)—C₁-C₃alkyl-, —C₀-C₃alkyl-N(R³)—C(O)-heterocyclyl-O-cycloalkyl-C₀-C₆alkyl-N(R³)—C(S)—C₁-C₃alkyl-, —C₀-C₃alkyl-N(R³)—C(S)-heterocyclyl-5-cycloalkyl-C₀-C₆alkyl-N(R³)—C(O)—C₁-C₃alkyl-, —C₀-C₃alkyl-N(R³)—C(S)-heterocyclyl-O-cycloalkyl-C₀-C₆alkyl-N(R³)—C(S)—C₁-C₃alkyl-, —C₀-C₃alkyl-N(R³)—C(O)-heterocyclyl-S-cycloalkyl-C₀-C₆alkyl-N(R³)—C(S)—C₁-C₃alkyl-, —C₀-C₃alkyl-N(R³)—C(S)-heterocyclyl-S-cycloalkyl-C₀-C₆alkyl-N(R³)—C(S)—C₁-C₃alkyl-, —C₀-C₃alkyl-S(O)₂-heterocyclyl-O-aryl-C₀-C₆alkyl-N(R³)—C(O)—C₁-C₃alkyl-, —C₀-C₃alkyl-S(O)₂-heterocyclyl-S-aryl-C₀-C₆alkyl-N(R³)—C(O)—C₁-C₃alkyl-, —C₀-C₃alkyl-S(O)₂-heterocyclyl-O-aryl-C₀-C₆alkyl-N(R³)—C(S)—C₁-C₃alkyl-, —C₁-C₃alkyl-S(O)₂-heterocyclyl-S-aryl-C₀-C₆alkyl-N(R³)—C(S)—C₁-C₃alkyl-, —C₀-C₃alkyl-S(O)₂-heterocyclyl-O-heteroaryl-C₀-C₆alkyl-N(R³)—C(O)—C₁-C₃alkyl-, —C₀-C₃alkyl-S(O)₂-heterocyclyl-S-heteroaryl-C₀-C₆alkyl-N(R³)—C(O)—C₁-C₃alkyl-, —C₀-C₃alkyl-S(O)₂-heterocyclyl-O-heteroaryl-C₀-C₆alkyl-N(R³)—C(S)—C₁-C₃alkyl-, —C₀-C₃alkyl-S(O)₂-heterocyclyl-S-heteroaryl-C₀-C₆alkyl-N(R³)—C(S)—C₁-C₃alkyl-, —C₀-C₃alkyl-S(O)₂-heterocyclyl-O-heterocyclyl-C₀-C₆alkyl-N(R³)—C(O)—C₁-C₃alkyl-, —C₀-C₃alkyl-S(O)₂-heterocyclyl-S-heterocyclyl-C₀-C₆alkyl-N(R³)—C(O)—C₁-C₃alkyl-, —C₀-C₃alkyl-S(O)₂-heterocyclyl-O-heterocyclyl-C₀-C₆alkyl-N(R³)—C(S)—C₁-C₃alkyl-, —C₀-C₃alkyl-S(O)₂-heterocyclyl-S-heterocyclyl-C₀-C₆alkyl-N(R³)—C(S)—C₁-C₃alkyl-, —C₀-C₃alkyl-S(O)₂-heterocyclyl-O-cycloalkyl-C₀-C₆alkyl-N(R³)—C(O)—C₁-C₃alkyl-, —C₀-C₃alkyl-S(O)₂-heterocyclyl-S-cycloalkyl-C₀-C₆alkyl-N(R³)—C(O)—C₁-C₃alkyl-, —C₀-C₃alkyl-S(O)₂-heterocyclyl-O-cycloalkyl-C₀-C₆alkyl-N(R³)—C(S)—C₁-C₃alkyl-, —C₀-C₃alkyl-S(O)₂-heterocyclyl-S-cycloalkyl-C₀-C₆alkyl-N(R³)—C(S)—C₁-C₃alkyl-, —C₀-C₃alkyl-heterocyclyl-N(R³)-aryl-C₀-C₆alkyl-N(R³)—C(O)—C₁-C₃alkyl-, —C₀-C₃alkyl-heterocyclyl-N(R³)-aryl-C₀-C₆alkyl-N(R³)—C(S)—C₁-C₃alkyl-, —C₀-C₃alkyl-heterocyclyl-N(R³)-heteroaryl-C₀-C₆alkyl-N(R³)— C(O)—C₁-C₃alkyl-, —C₁-C₃alkyl-heterocyclyl-N(R³)-heteroaryl-C₀-C₆alkyl-N(R³)—C(S)—C₁-C₃alkyl-, —C₀-C₃alkyl-heterocyclyl-N(R³)-heterocyclyl-C₀-C₆ alkyl-N(R³)—C(O)—C₁-C₃alkyl-, —C₀-C₃alkyl-heterocyclyl-N(R³)-heterocyclyl-C₀-C₆alkyl-N(R³)—C(S)—C₁-C₃alkyl-, —C₀-C₃alkyl-heterocyclyl-N(R³)-cycloalkyl-C₀-C₆alkyl-N(R³)—C(O)—C₁-C₃alkyl-, —C₀-C₃alkyl-heterocyclyl-N(R³)-cycloalkyl-C₀-C₆alkyl-N(R³)—C(S)—C₁-C₃alkyl-, —C₀-C₃alkyl-C(O)-heterocyclyl-N(R³)-aryl-C₀-C₆alkyl-N(R³)—C(O)—C₁-C₃alkyl-, —C₀-C₃alkyl-C(S)-heterocyclyl-N(R³)-aryl-C₀-C₆alkyl-N(R³)—C(O)—C₁-C₃alkyl-, —C₀-C₃alkyl-C(O)-heterocyclyl-N(R³)-aryl-C₀-C₆alkyl-N(R³)—C(S)—C₁-C₃alkyl-, —C₀-C₃alkyl-C(S)-heterocyclyl-N(R³)-aryl-C₀-C₆alkyl-N(R³)—C(S)—C₁-C₃alkyl-, —C₀-C₃alkyl-C(O)-heterocyclyl-N(R³)-heteroaryl-C₀-C₆alkyl-N(R³)—C(O)—C₁-C₃alkyl-, —C₀-C₃alkyl-C(S)-heterocyclyl-N(R³)-heteroaryl-C₀-C₆alkyl-N(R³)—C(O)—C₁-C₃alkyl-, —C₀-C₃alkyl-C(O)-heterocyclyl-N(R³)-heteroaryl-C₀-C₆alkyl-N(R³)—C(S)—C₁-C₃alkyl-, —C₀-C₃alkyl-C(S)-heterocyclyl-N(R³)-heteroaryl-C₀-C₆alkyl-N(R³)—C(S)—C₁-C₃alkyl-, —C₀-C₃alkyl-C(O)-heterocyclyl-N(R³)-heterocyclyl-C₀-C₆alkyl-N(R³)—C(O)—C₁-C₃alkyl-, —C₀-C₃alkyl-C(S)-heterocyclyl-N(R³)-heterocyclyl-C₀-C₆alkyl-N(R³)—C(O)—C₁-C₃alkyl-, —C₀-C₃alkyl-C(O)-heterocyclyl-N(R³)-heterocyclyl-C₀-C₆alkyl-N(R³)—C(S)—C₁-C₃alkyl-, —C₀-C₃alkyl-C(S)-heterocyclyl-N(R³)-heterocyclyl-C₀-C₆alkyl-N(R³)—C(S)—C₁-C₃alkyl-, —C₀-C₃alkyl-C(O)-heterocyclyl-N(R³)-cycloalkyl-C₀-C₆alkyl-N(R³)—C(O)—C₁-C₃alkyl-, —C₀-C₃alkyl-C(S)-heterocyclyl-N(R³)-cycloalkyl-C₀-C₆alkyl-N(R³)—C(O)—C₁-C₃alkyl-, —C₀-C₃alkyl-C(O)-heterocyclyl-N(R³)-cycloalkyl-C₀-C₆alkyl-N(R³)—C(S)—C₁-C₃alkyl-, —C₀-C₃alkyl-C(S)-heterocyclyl-N(R³)-cycloalkyl-C₀-C₆alkyl-N(R³)—C(S)—C₁-C₃alkyl-, —C₀-C₃alkyl-N(R³)—C(O)-heterocyclyl-N(R³)-aryl-C₀-C₆alkyl-N(R³)—C(O)—C₁-C₃alkyl-, —C₀-C₃alkyl-N(R³)—C(S)-heterocyclyl-N(R³)-aryl-C₀-C₆alkyl-N(R³)—C(O)—C₁-C₃alkyl-, —C₀-C₃alkyl-N(R³)—C(O)-heterocyclyl-N(R³)-aryl-C₀-C₆alkyl-N(R³)—C(S)—C₁-C₃alkyl-, —C₀-C₃alkyl-N(R³)—C(S)-heterocyclyl-N(R³)-aryl-C₀-C₆alkyl-N(R³)—C(S)—C₁-C₃alkyl-, —C₀-C₃alkyl-N(R³)—C(O)-heterocyclyl-N(R³)-heteroaryl-C₀-C₆alkyl-N(R³)—C(O)—C₁-C₃alkyl-, —C₀-C₃alkyl-N(R³)—C(S)-heterocyclyl-N(R³)-heteroaryl-C₀-C₆alkyl-N(R³)—C(O)—C₁-C₃alkyl-, —C₀-C₃alkyl-N(R³)—C(O)-heterocyclyl-N(R³)-heteroaryl-C₀-C₆alkyl-N(R³)—C(S)—C₁-C₃alkyl-, —C₀-C₃alkyl-N(R³)—C(S)-heterocyclyl-N(R³)-heteroaryl-C₀-C₆alkyl-N(R³)—C(S)—C₁-C₃alkyl-, —C₀-C₃alkyl-N(R³)—C(O)-heterocyclyl-N(R³)-heterocyclyl-C₀-C₆alkyl-N(R³)—C(O)—C₁-C₃alkyl-, —C₀-C₃alkyl-N(R³)—C(S)-heterocyclyl-N(R³)-heterocyclyl-C₀-C₆alkyl-N(R³)—C(O)—C₁-C₃alkyl-, —C₀-C₃alkyl-N(R³)—C(O)-heterocyclyl-N(R³)-heterocyclyl-C₀-C₆alkyl-N(R³)—C(S)—C₁-C₃alkyl-, —C₀-C₃alkyl-N(R³)—C(S)-heterocyclyl-N(R³)-heterocyclyl-C₀-C₆alkyl-N(R³)— C(S)—C₁-C₃alkyl-, —C₀-C₃alkyl-N(R³)—C(O)-heterocyclyl-N(R³)-cycloalkyl-C₀-C₆alkyl-N(R³)— C(O)—C₁-C₃alkyl-, —C₀-C₃alkyl-N(R³)—C(S)-heterocyclyl-N(R³)-cycloalkyl-C₀-C₆alkyl-N(R³)— C(O)—C₁-C₃alkyl-, —C₀-C₃alkyl-N(R³)—C(O)-heterocyclyl-N(R³)-cycloalkyl-C₀-C₆alkyl-N(R³)—C(S)—C₁-C₃alkyl-, —C₀-C₃alkyl-N(R³)—C(S)-heterocyclyl-N(R³)-cycloalkyl-C₀-C₆alkyl-N(R³)—C(S)—C₁-C₃alkyl-, —C₀-C₃alkyl-S(O)₂-heterocyclyl-N(R³)-aryl-C₀-C₆alkyl-N(R³)—C(O)—C₁-C₃alkyl-, —C₀-C₃alkyl-S(O)₂-heterocyclyl-N(R³)-aryl-C₀-C₆alkyl-N(R³)—C(S)—C₁-C₃alkyl-, —C₀-C₃alkyl-S(O)₂-heterocyclyl-N(R³)-heteroaryl-C₀-C₆alkyl-N(R³)—C(O)—C₁-C₃alkyl-, —C₀-C₃alkyl-S(O)₂-heterocyclyl-N(R³)-heteroaryl-C₀-C₆alkyl-N(R³)—C(S)—C₁-C₃alkyl-, —C₀-C₃alkyl-S(O)₂-heterocyclyl-N(R³)-heterocyclyl-C₀-C₆alkyl-N(R³)—C(O)—C₁-C₃alkyl-, —C₀-C₃alkyl —S(O)₂-heterocyclyl-N(R³)-heterocyclyl-C₀-C₆alkyl-N(R³)—C(S)—C₁-C₃alkyl-, —C₀-C₃alkyl-S(O)₂-heterocyclyl-N(R³)-cycloalkyl-C₀-C₆alkyl-N(R³)—C(O)—C₁-C₃alkyl-, —C₀-C₃alkyl-S(O)₂-heterocyclyl-N(R³)-cycloalkyl-C₀-C₆alkyl-N(R³)—C(S)—C₁-C₃alkyl-, —C₀-C₆alkyl-N(R³)—C(O)—C₀-C₆alkyl-heterocycloalkyl-C(O)—C₀-C₃alkyl-, —C₀-C₆alkyl-N(R³)—C(S)—C₀-C₆alkyl-heterocycloalkyl-C(O)—C₀-C₃alkyl-, —C₀-C₆alkyl-N(R³)—C(O)—C₀-C₆alkyl-heterocycloalkyl-C(S)—C₀-C₃alkyl-, —C₀-C₆alkyl-N(R³)—C(S)—C₀-C₆alkyl-heterocycloalkyl-C(S)—C₀-C₃alkyl-, —C₀-C₆alkyl-N(R³)—C(O)—C₀-C₆alkyl-heterocycloalkyl-C(O)—N(R³)—C₀-C₃alkyl-, —C₀-C₆alkyl-N(R³)—C(S)—C₀-C₆alkyl-heterocycloalkyl-C(O)—N(R³)—C₀-C₃alkyl-, —C₁-C₆alkyl-N(R³)—C(O)—C₀-C₆alkyl-heterocycloalkyl-C(S)—N(R³)—C₀-C₃alkyl-, —C₀-C₆alkyl-N(R³)—C(S)—C₀-C₆alkyl-heterocycloalkyl-C(S)—N(R³)—C₀-C₃alkyl-, —C₀-C₆alkyl-C(O)—C₀-C₆alkyl-heterocycloalkyl-C(O)—N(R³)—C₀-C₃alkyl-, —C₀-C₆alkyl-C(S)—C₀-C₆alkyl-heterocycloalkyl-C(O)—N(R³)—C₀-C₃alkyl-, —C₀-C₆alkyl-C(O)—C₀-C₆alkyl-heterocycloalkyl-C(S)—N(R³)—C₀-C₃alkyl-, —C₀-C₆alkyl-C(S)—C₀-C₆alkyl-heterocycloalkyl-C(S)—N(R³)—C₀-C₃alkyl-, —C₀-C₆alkyl-O—C₀-C₃alkyl-C(O)—N(R³)—C₀-C₃alkyl- and —C₀-C₆alkyl-O—C(S)—N(R³)—C₀-C₃alkyl-, wherein each alkyl, alkenyl, alkynyl, heteroalkyl, cycloalkyl, heterocycloalkyl, heterocyclyl, aryl and heteroaryl moiety of the aforementioned L are optionally substituted; wherein
Y is selected from the group consisting of H, alkyl, heteroalkyl, cycloalkyl, heterocyclyl, cycloalkylalkyl, heterocyclylalkyl, aryl, arylalkyl, heteroaryl, heteroarylalkyl, aryl-heteroaryl, aryl-heteroarylalkyl, heteroaryl-alkylaryl, aryl-aryl, aryl-arylalkyl, aryl-alkylaryl, aryl-C₀-C₃alkyl-O—C₀-C₃alkyl-aryl, aryl-C₀-C₃alkyl-S(O)₀₋₂—C₀-C₃alkyl-aryl, —C₀-C₃alkyl-S(O)₀₋₂—C₀-C₃alkyl-aryl, aryl-C₀-C₃alkyl-N(R³)—C₀-C₃alkyl-aryl, aryl-C₀-C₃alkyl-C(O)—N(R³)—C₀-C₃alkyl-aryl, aryl-C₀-C₃alkyl-C(S)—N(R³)—C₀-C₃alkyl-aryl, aryl-C₀-C₃alkyl-N(R³)—C(O)—C₀-C₃alkyl-aryl, aryl-C₀-C₃alkyl-N(R³)—C(S)—C₀-C₃alkyl-aryl, heteroaryl-heteroaryl, heteroaryl-aryl, heteroaryl-arylalkyl, aryl-alkylheteroaryl, heteroaryl-aryl-aryl, aryl-aryl-aryl, aryl-heteroaryl-aryl, aryl-heteroaryl-heteroaryl, heteroaryl-heteroaryl-heteroaryl, heteroaryl-heteroaryl-aryl, aryl-aryl-heteroaryl, heteroaryl-aryl-arylalkyl, aryl-aryl-alkylheteroaryl, heteroaryl-aryl-alkylaryl, aryl-aryl-alkylaryl, aryl-aryl-arylalkyl, aryl-aryl-heteroarylalkyl, heteroaryl-aryl-heteroaryl, heteroaryl-aryl-heteroarylalkyl, heteroaryl-aryl-alkylheteroaryl, heteroaryl-heteroarylalkyl, heteroaryl-alkylheteroaryl, heterocyclyl-heteroaryl, cycloalkyl-aryl, cycloalkyl-heteroaryl, heteroaryl-heterocyclyl, heteroaryl-cycloalkyl, aryl-cycloalkyl, heterocyclyl-aryl, aryl-heterocyclyl, heterocyclyl-alkyl-aryl, heterocyclyl-alkylheteroaryl, cycloalkyl-alkylaryl, cycloalkyl-alkylheteroaryl, aryl-alkyl-heterocyclyl, aryl-alkylcycloalkyl, heteroaryl-alkylcycloalkyl, heteroaryl-alkylheterocyclyl, arylalkyl-aryl, aryl-arylalkyl, aryl-heteroarylalkyl, heteroaryl-arylalkyl, heteroaryl-heteroarylalkyl, heteroaryl-C₀-C₃alkyl-O—C₀-C₃alkyl-aryl, heteroaryl-C₀-C₃alkyl-O—C₀-C₃alkyl-heteroaryl, aryl-C₀-C₃alkyl-O—C₀-C₃alkyl-heteroaryl, heteroaryl-C₀-C₃alkyl-N(R³)—C₀-C₃alkyl-aryl, aryl-C₀-C₃alkyl-N(R³)—C₀-C₃alkyl-heteroaryl, heteroaryl-C₀-C₃alkyl-N(R³)—C₀-C₃alkyl-heteroaryl, heteroaryl-C₀-C₃alkyl-C(O)—N(R³)—C₀-C₃alkyl-aryl, aryl-C₀-C₃alkyl-C(O)—N(R³)—C₀-C₃alkyl-heteroaryl, heteroaryl-C₀-C₃alkyl-C(O)—N(R³)—C₀-C₃alkyl-heteroaryl, aryl-C₀-C₃alkyl-C(S)—N(R³)—C₀-C₃alkyl-aryl, aryl-C₀-C₃alkyl-C(S)—N(R³)—C₀-C₃alkyl-heteroaryl, heteroaryl-C₀-C₃alkyl-C(S)—N(R³)—C₀-C₃alkyl-aryl, heteroaryl-C₀-C₃alkyl-C(S)—N(R³)—C₀-C₃alkyl-heteroaryl, heteroaryl-C₀-C₃alkyl-N(R³)—C(O)—C₀-C₃alkyl-aryl, heteroaryl-C₀-C₃alkyl-N(R³)—C(O)—C₀-C₃alkyl-heteroaryl, aryl-C₀-C₃alkyl-N(R³)—C(O)—C₀-C₃alkyl-heteroaryl, aryl-C₀-C₃alkyl-N(R³)—C(S)—C₀-C₃alkyl-aryl, aryl-C₀-C₃alkyl-N(R³)—C(S)—C₀-C₃alkyl-heteroaryl, heteroaryl-C₀-C₃alkyl-N(R³)—C(S)—C₀-C₃alkyl-aryl, heteroaryl-C₀-C₃alkyl-N(R³)—C(S)—C₀-C₃alkyl-heteroaryl, R³-heterocyclyl-C₀-C₃alkyl, R³-cycloalkyl-C₀-C₃alkyl, (R³)(R^{3a})N—C₂-C₄alkyl-O-aryl-, (R³)(R^{3a})N—C₂-C₄alkyl-S(O)₀₋₂-aryl-, (R³)(R^{3a})N—C₂-C₄alkyl-O-heteroaryl-, (R³)(R^{3a})N—C₂-C₄alkyl-S(O)₀₋₂-heteroaryl-, C₀-C₃alkyl-aryl-C₀-C₃alkyl, C₀-C₃alkyl-heteroaryl-C₀-C₃alkyl, aryl-C₁-C₃alkyl-heteroaryl, aryl-C₁-C₃alkyl-aryl, heteroaryl-C₁-C₃alkyl-aryl, heteroaryl-C₁-C₃alkyl-heteroaryl, R³-heterocyclyl-C₀-C₃alkyl-N(R³)—C(O)—N(R³)-heteroaryl-, R³-heterocyclyl-C₀-C₃alkyl-N(R³)—C(O)—N(R³)-aryl-, R³-cycloalkyl-C₀-C₃alkyl-N(R³)—C(O)—N(R³)-heteroaryl-, R³-cycloalkyl-C₀-C₃alkyl-N(R³)—C(O)—N(R³)-aryl-, R³-heterocyclyl-C₀-C₃alkyl-N(R³)—C(S)—N(R³)-heteroaryl-, R³-heterocyclyl-C₀-C₃alkyl-N(R³)—C(S)—N(R³)-aryl-, R³-cycloalkyl-C₀-C₃alkyl-N(R³)—C(S)—N(R³)-heteroaryl-, R³-cycloalkyl-C₀-C₃alkyl-N(R³)—C(S)—N(R³)-aryl-, heteroaryl-C(O)—C₀-C₆alkyl-heteroaryl-, heteroaryl-C(O)—C₀-C₆alkyl-aryl-, aryl-C(O)—C₀-C₆alkyl-aryl-, aryl-C(O)—C₀-C₆alkyl-heteroaryl-, heteroaryl-C(O)—N(R³)—C₀-C₆alkyl-aryl-, heteroaryl-C(O)—N(R³)—C₀-C₆alkyl-heteroaryl-, heteroaryl-S(O)₂—C₀-C₆alkyl-heteroaryl-, heteroaryl-S(O)₂—C₀-C₆alkyl-aryl-, aryl-S(O)₀₋₂—C₀-C₆alkyl-aryl, aryl-C₀-C₃alkyl-S(O)₀₋₂—C₀-C₆alkyl-heteroaryl, R³—O—C(O)—N(R³)—C₀-C₃alkyl-heteroaryl-, R³—O—C(O)—N(R³)—C₀-C₃alkyl-aryl-, R³—O—C(S)—N(R³)—C₀-C₃alkyl-heteroaryl-, R³—O—C(S)—N(R³)—C₀-C₃alkyl-aryl-, R³—C(O)-heterocyclyl-C₀-C₃alkyl-heteroaryl-, R³—C(O)-heterocyclyl-C₀-C₃alkyl-aryl-, R³—C(O)-cycloalkyl-C₀-C₃alkyl-heteroaryl-, R³—C(O)-cycloalkyl-C₀-C₃alkyl-aryl-, R³-heterocyclyl-C₀-C₃alkyl-N(R³)—C(O)—N(R³)—C₀-C₃alkyl-heteroaryl-, R³-heterocyclyl-C₀-C₃alkyl-N(R³)—S(O)₂—C₀-C₃alkyl-heteroaryl-, R³-heterocyclyl-C₀-C₃alkyl-N(R³)—S(O)₂—C₀-C₃alkyl-aryl-, R³-cycloalkyl-C₀-C₃alkyl-N(R³)—S(O)₂—C₀-C₃alkyl-heteroaryl-, R³-cycloalkyl-C₀-C₃alkyl-N(R³)—S(O)₂—C₀-C₃alkyl-aryl-, R³-heterocyclyl-C₀-C₃alkyl-N(R³)—C(O)—C₀-C₃alkyl-heteroaryl-, R³-heterocyclyl-C₀-C₃alkyl-N(R³)—C(O)—C₀-C₃alkyl-aryl-, R³-cycloalkyl-C₀-C₃alkyl-N(R³)—C(O)—C₀-C₃alkyl-heteroaryl-, R³-cycloalkyl-C₀-C₃alkyl-N(R³)—C(O)—C₀-C₃alkyl-aryl-, R³-heterocyclyl-C₀-C₃alkyl-N(R³)—C(S)—C₀-C₃alkyl-heteroaryl-, R³-heterocyclyl-C₀-C₃alkyl-N(R³)—C(S)—C₀-C₃alkyl-aryl-, R³-cycloalkyl-C₀-C₃alkyl-N(R³)—C(S)—C₀-C₃alkyl-heteroaryl-, R³-cycloalkyl-C₀-C₃alkyl-N(R³)—C(S)—C₀-C₃alkyl-aryl-, R³—C(O)—C₀-C₃alkyl-heteroaryl-, R³—C(O)—C₀-C₃alkyl-aryl-, heterocyclyl-C(O)—, an aromatic polycycle, a non-aromatic polycycle, a mixed aryl and non-aryl polycycle, a polyheteroaryl, a non-aromatic polyheterocycle, and a mixed aryl and non-aryl polyheterocycle, each of which is optionally substituted with one or more groups selected from R³, R⁴ or R⁷;
   or
Y is selected from the group consisting of
A^{2a}-aryl-C₀-C₃alkyl-N(R³)—C(O)—C₁-C₇alkyl-, wherein the C₁-C₇alkyl is optionally substituted with a moiety selected from the group consisting of —N(R³)—C(O)—C₁-C₅alkyl-(C₂-C₄alkenyl)₀₋₁C₁-C₃alkyl-O-A^{2b}, —N(R³)—C(O)—N(R³)—C₁-C₅alkyl-(C₂-C₄ alkenyl)₀₋₁-C₁-C₃alkyl-O-A^{2b}, —N(R³)—C(O)—O—C₁-C₅alkyl-(C₂-C₄alkenyl)₀₋₁-C₁-C₃alkyl-O-A^{2b}, —N(R³)—C₁-C₅alkyl-(C₂-C₄alkenyl)₀₋₁-C₁-C₃alkyl-O-A^{2b}, —N(R³)—S(O)₂—N(R³)—C₁-C₅alkyl-(C₂-C₄alkenyl)₀₋₁-C₁-C₃alkyl-O-A^{2b} and —N(R³)—S(O)₂—C₁-C₅alkyl-(C₂-C₄alkenyl)₀₋₁-C₁-C₃alkyl-O-A^{2b},
A^{2a}-heteroarylene-C₀-C₃alkyl-N(R³)—C(O)—C₁-C₇alkyl-, wherein the C₁-C₇alkyl is optionally substituted with a moeity selected from the group consisting of —N(R³)—C(O)—C₁-C₅alkyl-(C₂-C₄alkenyl)₀₋₁-C₃alkyl-O-A^{2b}, —N(R³)—C(O)—N(R³)—C₁-C₅alkyl-(C₂-C₄alkenyl)₀₋₁-C₁-C₃alkyl-O-A^{2b}, —N(R³)—C(O)—O—C₁-C₅alkyl-(C₂-C₄alkenyl)₀₋₁-C₁-C₃alkyl-O-A^{2b}, —N(R³)—C₁-C₅alkyl-(C₂-C₄alkenyl)₀₋₁-C₁-C₃alkyl-O-A^{2b}, —N(R³)—S(O)₂—N(R³)—C₁-C₅alkyl-(C₂-C₄alkenyl)₀₋₁-C₁-C₃alkyl-O-A^{2b} and —N(R³)—S(O)₂—C₁-C₅alkyl-(C₂-C₄alkenyl)₀₋₁-C₁-C₃alkyl-O-A^{2b} and
B²—B¹—N(R³)—C(O)—C₁-C₇alkyl-, wherein the C₁-C₇alkyl is optionally substituted with —NR³—B³ wherein the amine of B³ is connected with the acid of B² to form a peptide bond; and wherein
A^{2a} and A^{2b} together are a covalent bond and are attached to form a ring; and
B¹, B² and B³ are each independently a natural or synthetic amino acid and when any of B¹, B² and B³ are linked together they are linked together via a peptide bond;
each R³ and R^{3a} are independently selected from the group consisting of —H, —OH, —C(O)H, heterocyclyl, C₁-C₆alkyl, C₂-C₆alkenyl, C₂-C₆alkynyl, —C₂-C₄alkyl-OR^{a}, —C(O)—O—C₂-C₄alkyl-NR^{a}R^{a}, heteroalkyl, C₀-C₆alkylheteroaryl, C(O)CF₃, —C(O)—NH₂, —C(O)—NH—C₁-C₆alkyl, —NH₂, C₃-C₆cycloalkyl, —C₁-C₆alkylaryl, heteroaryl-aryl, aryl and alkylheteroaryl, wherein each alkyl, alkenyl, alkynyl, heteroalkyl, cycloalkyl, heterocyclyl, aryl and heteroaryl moiety is optionally substituted;
each R⁴ is independently selected from the group consisting of —H, —C(NR^{a})—N(R³)₂, C₁-C₆alkyl, C₂-C₆alkenyl, C₂-C₆alkynyl, —C₁-C₆alkyl-R^{a}, —C₀-C₆alkyl-O—R^{a}, —C₀-C₆alkyl-S(O)₀₋₂—R^{a}, —C₀-C₆alkyl-C(O)—OR^{a}, —C₀-C₆alkyl-C(O)—N(R³)(R^{3a}), —C₀-C₆alkyl-C(S)—N(R³)(R^{3a}), —CH═CH—C(O)—OR^{a}, —CH═CH—C(O)—N(R³)(R^{3a}), —CH═CH—C(S)—N(R³)(R^{3a}), —N(R³)—C(O)—CF₃, —N(R³)—C₂-C₆alkyl-N(R³)(R^{3a}), —C₀-C₆alkyl-N(R³)(R^{3a}), —N(R³)—C(O)—C₁-C₆alkyl-R³, —N(R³)—C(S)—C₁-C₆alkyl-R³, —N(R³)—S(O)₂—C₁-C₆alkyl-R³, —S(O)₂—N(R³)R^{3a}, —O—C₂-C₆alkyl-N(R³)(R^{3a}), —S(O)₀₋₂—C₂-C₆alkyl-N(R³)(R^{3a}), —S—R³, —S(O)₀₋₂—C₂-C₆alkyl-R³, —C₃-C₆cycloalkyl, —C₃-C₆cycloalkyl-R^{a}, heterocyclyl, —C₄-C₇heterocyclyl-R^{a}, —O—C₀-C₄alkyl-cycloalkyl, —S(O)₀₋₂—CO—C₄alkyl-cycloalkyl, —O—C₂-C₄alkyl-heterocyclyl (when the alkyl is linked via a N in the heterocyclyl), —O—C₀-C₄alkyl-heterocyclyl (when the alkyl is linked via a C in the heterocyclyl), —S(O)₀₋₁—C₀-C₄alkyl-heterocyclyl (when the alkyl is linked via a C in the heterocyclyl), —S(O)₀₋₁—C₂-C₄alkyl-heterocyclyl (when the alkyl is linked via a N in the heterocyclyl, —S(O)₂—C₀-C₄alkyl-heterocyclyl, —O—C₀-C₄alkyl-heterocyclyl-C(O)—OR^{a} (when the alkyl is linked via a C in the heterocyclyl, —O—C₂-C₄alkyl-heterocyclyl-C(O)—OR^{a} (when the alkyl is linked via a N in the heterocyclyl), —O-cycloalkyl-C(O)—OR^{a}, —O-aryl-C(O)—OR^{a}, —O-heteroaryl-C(O)—OR^{a}, —S(O)₀₋₁—C₀-C₄alkyl-heterocyclyl-C(O)—OR^{a} (when the alkyl is linked via a C in the heterocyclyl), —S(O)₀₋₁—C₂-C₄alkyl-heterocyclyl-C(O)—OR^{a} (when the alkyl is linked via a N in the heterocyclyl), —S(O)₂—C₀-C₄alkyl-heterocyclyl-C(O)—OR^{a}, —S(O)₀₋₂-cycloalkyl-C(O)—OR^{a}, —S(O)₀₋₂-aryl-C(O)—OR^{a}, —S(O)₀₋₂-heteroaryl-C(O)—OR^{a}, —O—C₀-C₄alkyl-aryl, —S(O)₀₋₂—C₀-C₄alkyl-aryl, —O—C₀-C₄alkyl-heteroaryl, —S(O)₀₋₂—C₀-C₄alkyl-heteroaryl, —O—C(O)—N(R³)—C₀-C₄alkyl-aryl, —O—C(S)—N(R³)—C₀-C₄alkyl-aryl, —O—C(O)—N(R³)—C₀-C₄alkyl-heteroaryl, —O—C(S)—N(R³)—C₀-C₄alkyl-heteroaryl, —O—C(O)—N(R³)—C₀-C₄alkyl-cycloalkyl, —O—C(S)—N(R³)—C₀-C₄alkyl-cycloalkyl, —O—C(O)—N(R³)—C₀-C₄alkyl-heterocyclyl, —O—C(S)—N(R³)—C₀-C₄alkyl-heterocyclyl, —O—C₀-C₄alkyl-heterocyclyl-aryl (when the alkyl is linked via a C in the heterocyclyl), —O—C₂-C₄alkyl-heterocyclyl-aryl (when the alkyl is linked via a N in the heterocyclyl), —O—C₀-C₄alkyl-heterocyclyl-heteroaryl (when the alkyl is linked via a C in the heterocyclyl), —O—C₂-C₄alkyl-heterocyclyl-heteroaryl (when the alkyl is linked via a N in the heterocyclyl), —O—C₀-C₄alkyl-heterocyclyl-cycloalkyl (when the alkyl is linked via a C in the heterocyclyl), —O—C₂-C₄alkyl-heterocyclyl-cycloalkyl (when the alkyl is linked via a N in the heterocyclyl), —O—C₀-C₄alkyl-heterocyclyl-heterocyclyl (when the alkyl is linked via a C in the heterocyclyl), —O—C₂-C₄alkyl-heterocyclyl-heterocyclyl (when the alkyl is linked via a N in the heterocyclyl), —S(O)₀₋₁—C₀-C₄alkyl-heterocyclyl-aryl (when the alkyl is linked via a C in the heterocyclyl), —S(O)₀₋₁—C₂-C₄alkyl-heterocyclyl-aryl (when the alkyl is linked via a N in the heterocyclyl), —S(O)₂—C₀-C₄alkyl-heterocyclyl-aryl, —S(O)₀₋₁—C₀-C₄alkyl-heterocyclyl-heteroaryl (when the alkyl is linked via a C in the heterocyclyl), —S(O)₀₋₁—C₂-C₄alkyl-heterocyclyl-heteroaryl (when the alkyl is linked via a N in the heterocyclyl), —S(O)₂—C₀-C₄alkyl-heterocyclyl-heteroaryl, —S(O)₀₋₁—C₀-C₄alkyl-heterocyclyl-cycloalkyl (when the alkyl is linked via a C in the heterocyclyl), —S(O)₀₋₁—C₂-C₄alkyl-heterocyclyl-cycloalkyl (when the alkyl is linked via a N in the heterocyclyl), —S(O)₂—C₀-C₄alkyl-heterocyclyl-cycloalkyl, —S(O)₀₋₁—C₀-C₄alkyl-heterocyclyl-heterocyclyl (when the alkyl is linked via a C in the heterocyclyl), —S(O)₀₋₁—C₂-C₄alkyl-heterocyclyl-heterocyclyl (when the alkyl is linked via a N in the heterocyclyl), —S(O)₂—C₀-C₄alkyl-heterocyclyl-heterocyclyl, —N(R³)—C₂-C₄alkyl-heterocyclyl, —N(R³)—C₂-C₄alkyl-cycloalkyl, —N(R³)—C₂-C₄alkyl-heteroaryl, —N(R³)—C₂-C₄alkyl-aryl, —N(R³)—C(O)—N(R³)—C₀-C₄alkyl-heterocyclyl-R³, —N(R³)—C(S)—N(R³)—C₀-C₄alkyl-heterocyclyl-R³, —N(R³)—C(O)—N(R³)—C₀-C₄alkyl-cycloalkyl-R³, —N(R³)—C(S)—N(R³)—C₀-C₄alkyl-cycloalkyl-R³, —N(R³)—C(O)—N(R³)—C₀-C₄alkyl-aryl-R³, —N(R³)—C(S)—N(R³)—C₀-C₄alkyl-aryl-R³, —N(R³)—C(O)—N(R³)—C₀-C₄alkyl-heteroaryl-R³, —N(R³)—C(S)—N(R³)—C₀-C₄alkyl-heteroaryl-R^{a}, —C₀-C₄alkyl-O—C(O)—R^{a}, —C₀-C₄alkyl-N(R³)—C(O)—O—R^{a}, —C₀-C₄alkyl-N(R³)—C(S)—O—R^{a}, —C₀-C₄alkyl-heterocyclyl-C(O)—O—R^{a}, —C₀-C₄alkyl-cycloalkyl-C(O)—O—R^{a}, —C₀-C₄alkyl-heteroaryl-C(O)—O—R^{a}, —C₀-C₄alkyl-aryl-C(O)—O—R^{a}, —N(R³)—C₂-C₄alkyl-heterocyclyl, —N(R³)—C₂-C₄alkyl-cycloalkyl, —N(R³)—C₂-C₄alkyl-heteroaryl, —N(R³)—C₂-C₄alkyl-aryl, F, Cl, Br, I, —CF₃, —SO₃H, —CN, aryl, heteroaryl, cycloalkyl and heterocyclyl, wherein each alkyl, alkenyl, alkynyl, cycloalkyl, heterocyclyl, aryl and heteroaryl moeity of the aforementioned R⁴ is optionally substituted;
each R⁷ and R^{7a} is independently selected from the group consisting of —H, C₁-C₆alkyl-, C₂-C₆alkenyl, C₂-C₆alkynyl, C₁-C₆heteroalkyl, R³—O—C₂-C₆alkyl-, R^{a}—S(O)₀₋₂—C₂-C₆alkyl-, N(R³)(R^{3a})—C₂-C₆alkyl-, a protecting group, C₁-C₆alkyl-O—C(O)—, aryl-C₀-C₄alkyl-O—C(O)—, heteroaryl-C₀-C₄alkyl-O—C(O)—, benzyl-O—C(O)—, heterocyclyl-C₁-C₆alkyl-, cycloalkyl-C₁-C₆alkyl-, heteroaryl-C₁-C₆alkyl-, aryl-C₁-C₆alkyl-, wherein each alkyl, alkenyl, alkynyl, heteroalkyl, aryl, heteroaryl, cycloalkyl, benzyl and heterocyclyl moiety is independently optionally substituted; provided that
R⁷ is —OR^{a} when attached to the N atom of an indolyl moiety; and
wherein in a —N(R³)(R^{3a}) group, optionally the R³ and R^{3a} together with the nitrogen atom to which they are attached form a heterocyclyl group;

with the proviso that Formula (I) excludes compounds of Formula (X-1), Formula (X-2), Formula (X-3) and Formula (X-4): 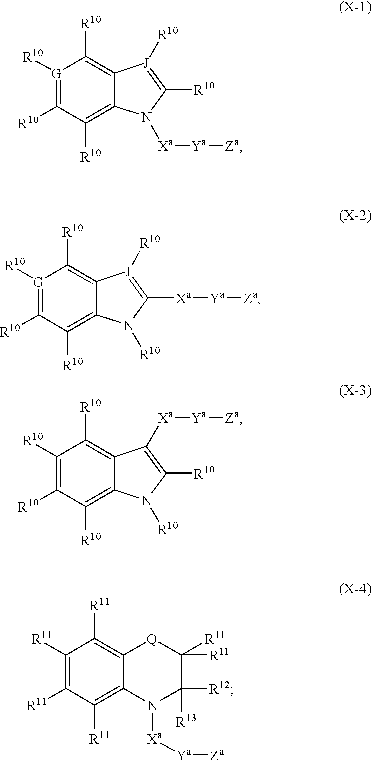
wherein,
G is N or C, subject to the proviso that R¹⁰ is absent when G is N;
J is N or C, subject to the proviso that R¹⁰ is absent when J is N;
Q is selected from the group consisting of S, O, SO₂ and NR¹¹;
X^{a} is —C(O)—, —S(O)₂— or a covalent bond;
Y^{a} is selected from the group consisting of alkyl, alkenyl, cycloalkyl, alkylcycloalkyl, alkylcycloalkylalkyl, alkyloxyalkyl, aryl, alkyaryl, alkylarylalkyl, arylalkyl, cycloalkylalkyl, alkylheterocycle, heterocyclealkyl, alkylheterocyclealkyl, heterocycle, aminoalkyl, oxyalkyl, aminoaryl and oxyaryl when 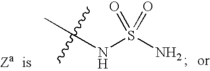
Y^{a} is selected from the group consisting of aminoalkyl and aminoaryl when Z^{a} is selected from the group consisting of 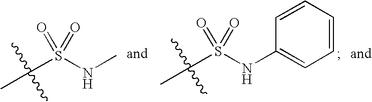
each of R¹⁰, R¹¹, R¹² and R¹³ is independently selected from the group consisting of R⁴; or
R¹² and R¹³ together are ═O or ═S; and

with the proviso that Formula (I) excludes compounds of Formula (X-5): 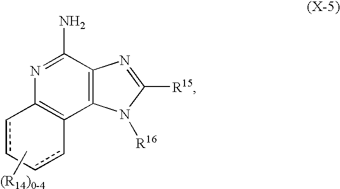
wherein each R¹⁴ is independently selected from the group consisting of C₁₋₁₀alkyl, C₁₋₁₀alkoxy, halogen and trifluoromethyl;
R¹⁵ is selected from the group consisting of R⁴;
R¹⁶ is -alkyl-N(R¹⁷)—S(O)₂—N(R¹⁹)—R¹⁸ or -alkenyl-N(R¹⁷)—S(O)₂—N(R¹⁹)—R¹⁸;
R¹⁷ is hydrogen or C₁₋₁₀alkyl;
R¹⁸ is selected from the group consisting of aryl, heteroaryl, heterocyclyl, alkyl or alkenyl, each of which is optionally substituted; and
R¹⁹ is selected from the group consisting of H and C₁₋₁₀alkyl, or R¹⁸ and R¹⁹ can combine to form a 3 to 7 membered heterocyclic or substituted heterocyclyl ring; and

with the proviso that Formula (I) excludes compounds of Formula (X-6): 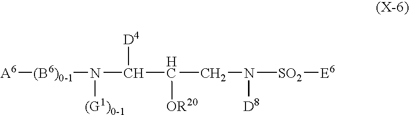
wherein
A⁶ is selected from the group consisting of C₃₋₇cycloalkyl, C₅₋₇cycloakenyl, C₆₋₁₀aryl and a 5-7 membered saturated or unsaturated heterocycle, each optionally substituted;
B⁶ is selected from the group consisting of —N(R²¹)—C(R²²)₂—C(O)—;
R²¹ is selected from H and C₁₋₄alkyl optionally substituted with a 3-7 membered saturated or unsaturated carbocyclic ring system or a 5-7 membered saturated or unsaturated heterocyclic ring;
each R²² is independently selected from the group consisting of H, C₃₋₇cycloalkyl, C₅₋₇cycloalkenyl, C₆₋₁₀aryl, 5-7 membered saturated or unsaturated heterocycle, C₁₋₆alkyl, C₂₋₆alkenyl, each of which except H is optionally substituted;
G¹ is selected from H and C₁₋₄alkyl;
D⁴ and D⁸ are independently selected from a 3-7 membered saturated or unsaturated optionally substituted carbocyclic ring system, a 5-7 membered saturated or unsaturated optionally substituted heterocyclic ring, C₁₋₆alkyl, which is optionally substituted with one or more groups selected from C₃₋₆cycloalkyl, —OR²¹, R²², —O-(3-7 membered saturated or unsaturated optionally substituted carbocyclic ring system), —O-(5-7 membered saturated or unsaturated optionally substituted heterocyclic ring), 3-7 membered saturated or unsaturated optionally substituted carbocyclic ring system, and 5-7 membered saturated or unsaturated optionally substituted heterocyclic ring; C₂₋₄alkenyl, which is substituted with one or more groups selected from C₃₋₆cycloalkyl, —OR²¹, —R²², —O-(3-7 membered saturated or unsaturated optionally substituted carbocyclic ring system), —O-(5-7 membered saturated or unsaturated optionally substituted heterocyclic ring), 3-7 membered saturated or unsaturated optionally substituted carbocyclic ring system, and 5-7 membered saturated or unsaturated optionally substituted heterocyclic ring; C₃₋₆cycloalkyl, which is optionally substituted with or fused to a 5-7 membered saturated or unsaturated optionally substituted heterocyclic ring or a 3-7 membered saturated or unsaturated optionally substituted carbocyclic ring system; and C₅₋₆cycloalkenyl, optionally substituted with or fused to a 3-7 membered saturated or unsaturated optionally substituted carbocyclic ring system, or a 5-7 membered saturated or unsaturated optionally substituted heterocyclic ring; 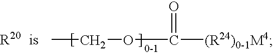
R²⁴ is C(R²¹)2, O or N(R²¹);
M⁴ is selected from the group consisting of H, optionally substituted C₁₋₁₂alkyl, optionally substituted C₂₋₁₂alkenyl, a 5-6 membered saturated or unsaturated optionally substituted carbocyclic or heterocyclic ring, an 8-10 membered saturated or unsaturated optionally substituted bicyclic ring system, wherein 1 to 4-CH₂ radicals of the alkyl or alkenyl group is optionally replaced by a heteroatom group selected from O, S, S(O), S(O)₂ and N(R²¹); and
E⁶ is selected from the group consisting of —O—R²² and —N(R²¹)(R²²); and

with the proviso that Formula (I) excludes compounds of Formula (X-7): 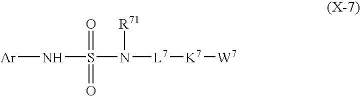
wherein,
Ar is selected from the group consisting of a mono-cyclic aryl, a mono-cyclic heteroaryl, a bicyclic aryl and a bicyclic heteroaryl;
R⁷¹ is selected from the group consisting of H and C₁₋₃ straight chained alkyl, wherein the alkyl is optionally substituted with a group selected from H, OR⁷⁵, CN, C₁₋₆alkyl, CH₂OR⁷⁵, CON(R⁷⁵)₂, CO₂R⁷⁵, phenyl, pyridyl, thiophenyl, and naphthyl;
each R⁷⁵ is independently selected from the group consisting of H, C₁₋₃alkyl, C₁₋₃monohaloalkyl and C₁₋₃polyhaloalkyl;
L⁷ is selected from the group consisting of —C₃₋₉alkyl-, —C₃₋₉alkenyl-, —C₃₋₉alkynyl-, 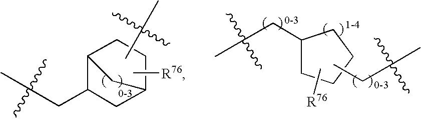
   and optionally substituted 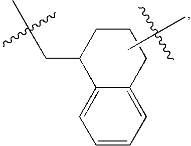
   wherein one dashed line is a double bond and the other dashed line is a single bond;
each R⁷⁶ is independently selected from the group consisting of H, CN, OR⁷⁵, C₁₋₅alkyl, CH₂OR⁷⁵, CON(R⁷⁵)₂, CO₂R⁷⁵, phenyl, pyridyl, thiophenyl and naphthyl, wherein the phenyl, pyridyl, thiophenyl and naphthyl is substituted with H, F, Cl, Br, I, CF₃, C₁₋₄alkyl, C₁₋₄alkoxy, C₁₋₄alkylthio or NO₂,
K⁷ is selected from the group consisting of —CH₂—NR⁷¹⁰—CHR⁷⁷—(CH₂)₀₋₃—, —CH₂—NR⁷¹⁰—CO—(CH₂)₀₋₃—, —CH₂—NH—CO—NH—(CH₂)₀₋₃—, —CO—NH—CHR⁷⁷—(CH₂)₀₋₃—, —CH₂—NR⁷¹⁰—CO—CHR⁷⁷—(CH₂)₀₋₃—, —CH₂—NR⁷¹⁰—CS—(CH₂)₀₋₃—, —CH₂—NH—CS—NH—(CH₂)₁₋₄, —CS—NH—CHR⁷⁷—(CH₂)₀₋₃—, —CH₂—NR⁷¹⁰—CS—CHR⁷⁷—(CH₂)₀₋₃— and —CH₂—N═CSR⁷¹—NH—(CH₂)₀₋₃—;
R⁷⁷ is selected from the group consisting of H, C₁₋₆alkyl, CH₂OR⁷⁵, —(CH₂)₀₋₂NHCO₂R⁷⁵, —(CH₂)₀₋₂NHSO₂R⁷⁵, CH₂N(R⁷¹¹)₂, phenyl, pyridyl, thiophenyl and naphthyl,
W⁷ is selected from the group consisting of a mono-cyclic aryl, a mono-cyclic heteroaryl, a bicyclic aryl, a bicyclic heteroaryl, a fused aryl-cycloalkyl, and a fused aryl-heterocyclyl, each of which is optionally substituted;
R⁷¹⁰ is H or C₁₋₆alkyl; and
R⁷¹¹ is selected from the group consisting of H, COR⁷⁵, COR⁷¹², SO₂R⁷⁵ and SO₂R⁷¹²; wherein
R⁷¹² is selected from the group consisting of phenoxy, phenyl, pyridyl, thiophenyl, and naphthyl, wherein the phenoxy, phenyl, pyridyl, thiophenyl and naphthyl is substituted with H, F, Cl, Br, I, CF₃, C₁₋₄alkyl, C₁₋₄alkoxy, C₁₋₄alkylthio, NO₂, phenyl, pyridyl, and thiophenyl; and

with the proviso that Formula (I) excludes the compounds: 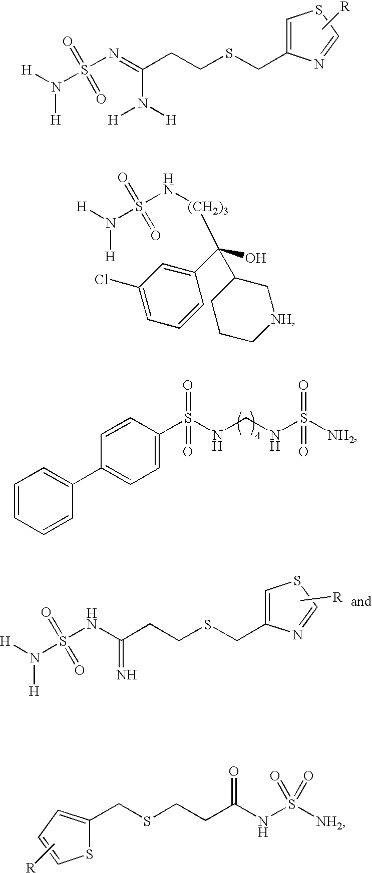
wherein R is a substituent; and

with the proviso that Formula (I) excludes compounds of Formula (X-8) and Formula (X-9): 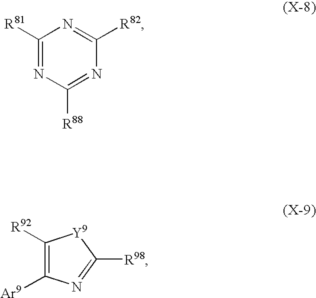
wherein
R⁸¹ is selected from the group consisting of F, Cl, Br, I, NR⁸³R⁸⁴, phenyl and heteroaryl, wherein the phenyl and heteroaryl may be substituted with one or more of F, Cl, Br, I, —CN, —NO₂, —NR⁸⁵R⁸⁶, —SO₂R⁸⁵, —(CH₂)₀₋₆C(Y⁸)R⁸⁷, —(CH₂)₀₋₆—Y⁸R⁸⁵, —(CH₂)₀₋₆C(Y⁸)NR⁸⁵R⁸⁶, —(CH₂)₀₋₆NR⁸⁵C(Y⁸)R⁸⁵, —(CH₂)₀₋₆CO₂R⁸⁵, —(CH2)₀₋₆SO₂NR⁸⁵R⁸⁶, a straight chained or branched C₁₋₇alkyl, monofluoroalkyl, polyfluoroalkyl, aminoalkyl, C₂₋₇alkenyl, C₂₋₇alkynyl, C₃₋₇cycloalkyl and C₃₋₇cycloalkenyl;
R⁸² is NR⁸³R⁸⁴;
R⁸³ is selected from the group consisting of H, —(CH₂)₂₋₄Y⁸R⁸⁵, —(CH₂)₁₋₄C(Y⁸)NR⁸⁵R⁸⁶, —(CH₂)₂₋₄NR⁸⁵C(Y⁸)R⁸⁵, —(CH₂)₁₋₄C(Y⁸)R⁸⁷, —(CH₂)₁₋₄CO₂R⁸⁵, —(CH₂)₂₋₄NR⁸⁵R⁸⁶, —(CH₂)₂₋₄CN, —C(Y⁸)R⁸⁵, —C(Y⁸)NR⁸⁵R⁸⁶, —CO₂R⁸⁵, straight chained or branched C₁₋₇alkyl, C₂₋₇alkenyl, C₂₋₇alkynyl, C₃₋₇cycloalkyl, C₃₋₇cycloalkenyl, phenyl, C₁₋₆-phenylalkyl and C₁₋₆heteroarylalkyl, wherein the phenyl, C₁₋₆phenylalkyl and C₁₋₆heteroarylalkyl may be substituted with one or more of F, Cl, Br, I, —CN, —NO₂, —NR⁸⁵R⁸⁶, —SO²R⁸⁵, —(CH₂)₀₋₆C(Y⁸)R⁸⁷, —(CH₂)₀₋₆—Y⁸R⁸⁵, —(CH₂)₀₋₆C(Y⁸)NR⁸⁵R⁸⁶, —(CH₂)₀₋₆NR⁸⁵C(Y⁸)R⁸⁵, —(CH₂)₀₋₆CO₂R⁸⁵, —(CH₂)₀₋₆SO₂NR⁸⁵R⁸⁶, a straight chained or branched C₁₋₇alkyl, monofluoroalkyl, polyfluoroalkyl, aminoalkyl, C₂₋₇alkenyl, C₂₋₇alkynyl, C₃₋₇cycloalkyl and C₃₋₇cycloalkenyl;
R⁸⁴ is selected from the group consisting of H, —(CH₂)₂₋₄Y⁸R⁸⁵, —(CH₂)₁₋₄C(Y⁸)NR⁸⁵R⁸⁶, —(CH₂)₂₋₄NR⁸⁵C(Y⁸)R⁸⁵, —(CH₂)₁₋₄C(Y⁸)R⁸⁷, —(CH₂)₁₋₄CO₂R⁸⁵, —(CH₂)₂₋₄NR⁸⁵R⁸⁶, —(CH₂)₂₋₄CN, straight chained or branched C₁₋₇alkyl, C₂₋₇alkenyl, C₂₋₇alkynyl, C₃₋₇cycloalkyl, C₃₋₇cycloalkenyl, phenyl, C₁₋₆phenylalkyl, wherein the phenyl and C₁₋₆phenylalkyl may be substituted with one or more of F, Cl, Br, I, —CN, —NO₂, —NR⁸⁵R⁸⁶, —SO₂R⁸⁵, —(CH₂)₀₋₆C(Y⁸)R⁸⁷, —(CH₂)₀₋₆—Y⁸R⁸⁵, —(CH₂)₀₋₆C(Y⁸)NR⁸⁵R⁸⁶, —(CH₂)₀₋₆NR⁸⁵C(Y⁸)R⁸⁵, —(CH₂)₀₋₆CO₂R⁸⁵, —(CH₂)₀₋₆SO₂NR⁸⁵R⁸⁶, a straight chained or branched C₁₋₇alkyl, monofluoroalkyl, polyfluoroalkyl, aminoalkyl, C₂₋₇alkenyl, C₂₋₇alkynyl, C₃₋₇cycloalkyl and C₃₋₇cycloalkenyl; or
R⁸³ and R⁸⁴ taken together with the nitrogen atom to which they are attached are 1-azetidinyl, 1-pyrrolidinyl, 1-piperidinyl or 1H-azepanyl, each of which is optionally substituted;
or R⁸³ and R⁸⁴ taken together with the nitrogen atom to which they are attached are morpholinyl, thiomorpholinyl, [1,4]oxazepanyl, [1,4]thiazepanyl, piperazinyl or [1,4]diazepanyl, each of which is optionally substituted;
each of R⁸⁵, R⁸⁶ and R⁸⁷ is independently selected from the group consisting of H and straight chained or branched C₁₋₇alkyl;
Y⁸ is O or S;
Y⁹ is selected from the group consisting of O, S and NH;
R⁸⁸ is selected from the group consisting of 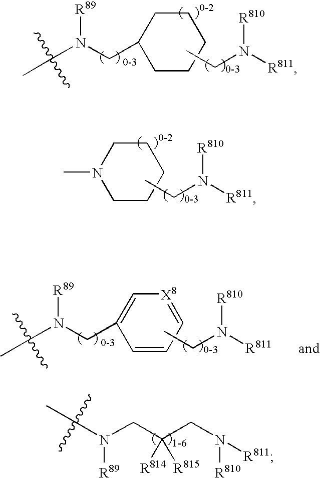
R⁹⁸ is selected from the group consisting of 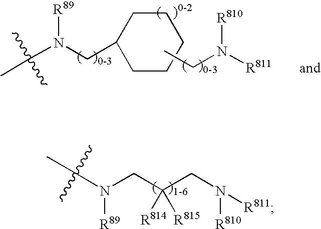
each of R⁸⁹ and R¹¹⁰ is independently H or a straight chained or branched C₁₋₄alkyl;
R⁸¹¹ is —SO₂—NR⁸³R⁸⁴;
R⁸¹⁴ is selected from the group consisting of H, straight chained or branched C₁₋₇alkyl, F and —(CH₂)₀₋₆OR⁸⁵;
R⁸¹⁵ is selected from the group consisting of H, straight chained or branched C₁₋₇alkyl and F;
Ar⁹ is a heteroaryl ring;
R⁹² is selected from the group consisting of H, straight chained or branched C₁₋₄alkyl, —(CH₂)₁₋₄OR⁹⁵, phenyl optionally substituted with one or more of F, Cl, Br, CF₃, CN, NO₂, NR⁹⁵R⁹⁶, —SO₂R⁹⁵, —(CH₂)₀₋₆OR⁹⁵ and straight chained or branched C₁₋₄alkyl;
X⁸ is N or C; and
R⁹⁵ and R⁹⁶ are independently H or straight chained or branched C₁₋₇alkyl; and

with the proviso that Formula (I) excludes those compounds wherein when: 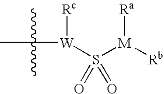
is NR^{c}—SO₂—NR^{a}R^{b}, wherein
each of R^{a}, R^{b} and R^{a} is independently selected from the group consisting of H and C₁₋₄alkyl; then Y-L-Z- is not 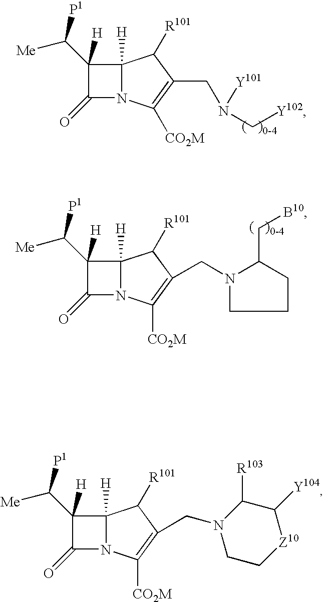
wherein
P¹ is H or hydroxy;
R¹⁰¹ is H or methyl;
Y¹⁰¹ is H or C₁₋₄alkyl;
the group CO₂M is selected from the group consisting of a carboxylic acid, a carboxylate anion, a pharmaceutically acceptable ester group, and a carboxylic acid protected by a protecting group;
Y¹⁰² is —C(O)— or absent and Y¹⁰² is the point of attachment to W of Formula (I);
Z¹⁰ is —O—, —S—, —N(H)— or —N(C₁-C₄alkyl)-;
B¹⁰ is —C(O)— or absent and B¹⁰ is the point of attachment to W of Formula (I); and
one of Y¹⁰³ and Y¹⁰⁴ is selected from the group consisting of H and —(CH₂)₀₋₄-A¹¹, wherein A¹⁰ is selected from the group consisting of —N(R¹⁰²)₂, —CO₂R¹⁰², —CO—N(R¹⁰²)₂, —CO—NR¹⁰² SO₂N(R¹⁰²)₂, —NR¹⁰²SO₂N(R¹⁰²)₂, —NH—C(═NR¹⁰²)—N(R¹⁰²)₂, and —S—C(═NR¹⁰²)—N(R¹⁰²)₂, and the other one of Y¹⁰³ and Y¹⁰⁴ is the point of attachment to W of Formula (I) and is either —C(O)— or absent, wherein each R¹⁰² is independently H or C₁₋₄alkyl; and

with the proviso that Formula (I) excludes those compounds wherein Y-L-Z- is selected from the group consisting of aryl-(CH₂)₂—, heteroaryl-(CH₂)₂—, heterocycle-(CH₂)₂— and cycloalkyl-(CH₂)₂—.

In a preferred embodiment of the present invention, each alkyl, alkenyl, alkynyl, heteroalkyl, cycloalkyl, heterocyclyl, aryl and heteroaryl moiety of Y, L, Z, R^{a}, R^{b}, R^{c}, R³ and R^{3a} is independently optionally substituted with one or more groups independently selected from

In a preferred embodiment of the present invention, each alkyl, alkenyl, alkynyl, heteroalkyl, cycloalkyl, heterocyclyl, aryl and heteroaryl moiety of Y, L, Z, R^{a}, R^{b}, R^{c}, R³ and R^{3a} is independently optionally substituted with one or more groups independently selected from oxo, —OH, —CN, C₁-C₆alkyl, C₁-C₆alkoxy, —NO₂, —N(R^{a})₂, —N(R⁷)(R^{7a}), R⁴, halo, —SH, —S—C₁-C₆alkyl, —S(O)—C₁-C₆alkyl, —S—C(O)—C₁-C₆alkyl and mono- to per-halogenated C₁-C₆alkyl.

In a preferred embodiment of the present invention, a C₁-C₆alkyl moiety of an R⁴ is optionally substituted with a substituent selected from the group consisting of —OH, —NO₂ and C₀-C₆ alkyl-C(O)—N(R³)(R^{3a}).

In a preferred embodiment of the present invention each alkyl, alkenyl, alkynyl, heteroalkyl, aryl, heteroaryl, cycloalkyl and heterocyclyl moiety of Z is independently optionally substituted with one or more substituents independently selected from the group consisting of oxo, —OH, —CN, C₁-C₆alkyl, C₁-C₆alkoxy, —NO₂, —N(R³)(R^{3a}), halo, —SH and mono- to per-halogenated C₁-C₆alkyl.

In a preferred embodiment of the present invention, L is selected from the group consisting of
—C₀-C₆alkyl-N(R³)—C₀-C₃alkyl-, wherein when the —C₀-C₆alkyl is —C₁-C₆alkyl it is optionally substituted with a substituent selected from the group consisting of —C₁-C₃alkyl-OR^{a}, —C₁-C₃alkyl-N(R³)(R^{3a}), —C₀-C₃alkyl-C(O)OR^{a} and C₀-C₃alkyl-C(O)—N(R³)(R^{3a});
—C₀-C₆alkyl-N(R³)—C(O)—C₀-C₃alkyl-, wherein when the —C₀-C₆alkyl is C₁-C₆alkyl it is optionally substituted with a substituent selected from the group consisting of —C₁-C₃alkyl-OR^{a}, —C₁-C₃alkyl-NR³R^{3a}, —C₀-C₃alkyl-C(O)OR^{a} and C₀-C₃alkyl-C(O)—N(R³)(R^{3a});
—C₀-C₆alkyl-N(R³)—C(O)—C₀-C₃alkyl-, wherein the —C₀-C₃alkyl is C₁-C₃alkyl it is optionally substituted with a substituent selected from the group consisting of —N(R³)—C(O)—C₀-C₃alkyl-Y, —N(R³)—C(S)—C₀-C₃alkyl-Y, —C(O)—N(R³)—C₀-C₃alkyl-Y, —C(S)—N(R³)—C₀-C₃alkyl-Y, —N(R³)—C(O)—C₀-C₃alkyl-C₄-C₆cycloalkyl-, —N(R³)—C(S)—C₀-C₃alkyl-C₄-C₆cycloalkyl-, —N(R³)—C₀-C₃alkyl-Y, —N(R³)(R^{3a}), —N(R³)—C₀-C₃alkyl-C₄-C₆heterocyclyl, —N(R³)—C₂-C₃alkyl-N(R³)(R³a), —N(R³)—C₂-C₃alkyl-OR^{a}, —N(R³)—C₀-C₃alkyl-C₀-C₃heteroalkyl-Y, —N(R³)—C(O)—O—C₀-C₃alkyl-Y, —N(R³)—C(S)—O—C₀-C₃alkyl-Y, —N(R³)—S(O)₂—C₀-C₃alkyl-Y, —N(R³)—C(O)—N(R³)—C₀-C₃alkyl-Y, —N(R³)—C(S)—N(R³)C₀-C₀-C₃alkyl-Y, —N(R³)—C(O)—C₀-C₃alkyl-C₀-C₃heterocyclyl, —N(R³)—C(S)—C₀-C₃alkyl-C₀-C₃heterocyclyl, —N(R³)—C(O)—C₀-C₃alkyl-C₀-C₃heterocyclyl-Y, —N(R³)—C(S)—C₀-C₃alkyl-C₀-C₃heterocyclyl-Y and —N(R³)—S(O)₂—N(R³)—C₀-C₃alkyl-Y;
—C₀-C₆alkyl-N(R³)—C(S)—C₀-C₃alkyl-, wherein the —C₀-C₃alkyl is C₁-C₃alkyl it is optionally substituted with a substituent selected from the group consisting of —N(R³)—C(O)—C₀-C₃alkyl-Y, —N(R³)—C(S)—C₀-C₃alkyl-Y, —C(O)—N(R³)—C₀-C₃alkyl-Y, —C(S)—N(R³)—C₀-C₃alkyl-Y, —N(R³)—C(O)—C₀-C₃alkyl-C₄-C₆cycloalkyl-, —N(R³)—C(S)—C₀-C₃alkyl-C₄-C₆cycloalkyl-, —N(R³)—C₀-C₃alkyl-Y, —N(R³)(R^{3a}), —N(R³)—C₀-C₃alkyl-C₄-C₆heterocyclyl, —N(R³)—C₂-C₃alkyl-N(R³)(R^{3a}), —N(R³)—C₂-C₃alkyl-OR^{a}, —N(R³)—C₀-C₃alkyl-C₀-C₃heteroalkyl-Y, —N(R³)—C(O)—O—C₀-C₃alkyl-Y, —N(R³)—C(S)—O—C₀-C₃alkyl-Y, —N(R³)—S(O)₂—C₀-C₃alkyl-Y, —N(R³)—C(O)—N(R³)—C₀-C₃alkyl-Y, —N(R³)—C(S)—N(R³)—C₀-C₃alkyl-Y, —N(R³)—C(O)—C₀-C₃alkyl-C₀-C₃heterocyclyl, —N(R³)—C(S)—C₀-C₃alkyl-C₀-C₃heterocyclyl, —N(R³)—C(O)—C₀-C₃alkyl-C₀-C₃heterocyclyl-Y, —N(R³)—C(S)—C₀-C₃alkyl-C₀-C₃heterocyclyl-Y and —N(R³)—S(O)₂—N(R³)—C₀-C₃alkyl-Y;
—C₀-C₆alkyl-C(O)—C₀-C₃alkyl-, wherein when the —C₀-C₆alkyl is C₁-C₃alkyl it is optionally substituted with a substituent selected from the group consisting of —N(R³)—C(O)—C₀-C₃alkyl-Y, —N(R³)—C(S)—C₀-C₃alkyl-Y, —C(O)—N(R³)(R^{3a}), —C(S)—N(R³)(R^{3a}), —C(O)—N(R³)—C₀-C₃alkyl-Y, —C(S)—N(R³)—C₀-C₃alkyl-Y, —N(R³)—C(O)—C₀-C₃alkyl-C₄-C₆cycloalkyl-, —N(R³)—C(S)—C₀-C₃alkyl-C₄-C₆cycloalkyl-, —N(R³)—C₀-C₃alkyl-Y, —N(R³)(R^{3a}), —N(R³)—C₀-C₃alkyl-C₄-C₆heterocyclyl, —N(R³)—C₂-C₃alkyl-N(R³)(R^{3a}), —N(R³)—C₂-C₃alkyl-OR^{a}—, —N(R³)—C₀-C₃heteroalkyl-Y, —N(R³)—C(O)—O—C₀-C₃alkyl-Y, —N(R³)—C(S)—O—C₀-C₃alkyl-Y, —N(R³)—S(O)₂—C₀-C₃alkyl-Y, —N(R³)—C(O)—N(R³)—C₀-C₃alkyl-Y, —N(R³)—C(S)—N(R³)—C₀-C₃alkyl-Y, —N(R³)—C(O)—C₀-C₃alkyl-C₀-C₃heterocyclyl, —N(R³)—C(S)—C₀-C₃alkyl-C₀-C₃heterocyclyl, —N(R³)—C(O)—C₀-C₃alkyl-C₀-C₃heterocyclyl-Y, —N(R³)—C(S)—C₀-C₃alkyl-C₀-C₃heterocyclyl-Y and —N(R³)—S(O)₂—N(R³)—C₀-C₃alkyl-Y;
—C₀-C₆alkyl-C(S)—C₀-C₃alkyl-, wherein when the —C₀-C₆alkyl is C₁-C₃alkyl it is optionally substituted with a substituent selected from the group consisting of —N(R³)—C(O)—C₀-C₃alkyl-Y, —N(R³)—C(S)—C₀-C₃alkyl-Y, —C(O)—N(R³)(R^{3a}), —C(S)—N(R³)(R^{3a}), —C(O)—N(R³)—C₀-C₃alkyl-Y, —C(S)—N(R³)—C₀-C₃alkyl-Y, —N(R³)—C(O)—C₀-C₃alkyl-C₄-C₆cycloalkyl-, —N(R³)—C(S)—C₀-C₃alkyl-C₄-C₆cycloalkyl-, —N(R³)—C₀-C₃alkyl-Y, —N(R³)(R^{3a}), —N(R³)—C₀-C₃alkyl-C₄-C₆heterocyclyl, —N(R³)—C₂-C₃alkyl-N(R³)(R^{3a}), —N(R³)—C₂-C₃alkyl-OR^{a}—, —N(R³)—C₀-C₃heteroalkyl-Y, —N(R³)—C(O)—O—C₀-C₃alkyl-Y, —N(R³)—C(S)—O—C₀-C₃alkyl-Y, —N(R³)—S(O)₂—C₀-C₃alkyl-Y, —N(R³)—C(O)—N(R³)—C₀-C₃alkyl-Y, —N(R³)—C(S)—N(R³)—C₀-C₃alkyl-Y, —N(R³)—C(O)—C₀-C₃alkyl-C₀-C₃heterocyclyl, —N(R³)—C(S)—C₀-C₃alkyl-C₀-C₃heterocyclyl, —N(R³)—C(O)—C₀-C₃alkyl-C₀-C₃heterocyclyl-Y, —N(R³)—C(S)—C₀-C₃alkyl-C₀-C₃heterocyclyl-Y and —N(R³)—S(O)₂—N(R³)—C₀-C₃alkyl-Y;
—C₀-C₆alkyl-, wherein when the —C₀-C₆alkyl is C₁-C₆alkyl it is optionally substituted with a substituent selected from the group consisting of —N(R³)—C(O)—C₀-C₃alkyl-Y, —N(R³)—C(O)—C₀-C₃alkyl-heterocyclyl and —N(R³)—C(O)—C₀-C₃alkyl-cycloalkyl;
—C₀-C₆alkyl-C(O)—N(R³)—C₀-C₃alkyl-, wherein when the —C₀-C₃alkyl is C₁-C₃alkyl it is optionally substituted with a substituent selected from the group consisting of —C(O)—N(R³)—C₀-C₃alkyl-Y, —C(O)-heterocyclyl, —C(O)—N(R³)(R^{3a}), aryl-aryl, aryl-heteroaryl, -heteroaryl-aryl, heteraryl-heteroaryl, heteroaryl, heterocyclyl-heteroaryl and heterocyclyl;
—C₀-C₆alkyl-heteroalkyl-C₀-C₆alkyl-C(O)—N(R³)—C₀-C₃alkyl-, wherein when the —C₀-C₃alkyl is C₁-C₃alkyl it is optionally substituted with a substituent selected from the group consisting of —C(O)—N(R³)—C₀-C₃alkyl-Y, —C(O)-heterocyclyl, —C(O)—N(R³)(R^{3a}), aryl-aryl, aryl-heteroaryl, -heteroaryl-aryl, heteraryl-heteroaryl, heteroaryl, heterocyclyl-heteroaryl and heterocyclyl;
—C₀-C₆alkyl-C(O)—N(R³)—C₀-C₃alkyl-, wherein when the —C₀-C₆alkyl is C₁-C₆alkyl it is optionally substituted with a substituent selected from the group consisting of —N(R⁷)(R^{7a}), —N(R³)(R^{3a}), —N(R³)—C(O)—C₀-C₃alkyl-Y, —N(R³)—C(O)—O—C₀-C₃alkyl-Y, —N(R³)—C(O)—N(R³)—C₀-C₃alkyl-Y and —N(R³)—S(O)₂—C₀-C₃alkyl-Y;
—C₀-C₆alkyl-heteroaryl-C₀-C₃alkyl-, wherein when the —C₀-C₃alkyl is C₁-C₃alkyl it is optionally substituted with a substituent selected from the group consisting of —C₀-C₃alkyl-N(R³)—C(O)—C₀-C₃alkyl-N(R³)(R^{3a}), —N(R³)—C(O)—C₀-C₃alkyl-Y, —N(R³)—C(O)—O—C₀-C₃alkyl-Y, —N(R³)—C(O)—N(R³)—C₀-C₃alkyl-Y and —N(R³)—S(O)₂—C₀-C₃alkyl-Y;
—C₀-C₆alkyl-aryl-C₀-C₃alkyl-, wherein when the —C₀-C₃alkyl is C₁-C₃alkyl it is optionally substituted with a substituent selected from the group consisting of —N(R³)—C(O)—C₀-C₃alkyl-Y, —N(R³)—C(O)—C₀-C₃alkyl-Y, —N(R³)—C(O)—O—C₀-C₃alkyl-Y, —N(R³)—C(O)—N(R³)—C₀-C₃alkyl-Y and —N(R³)—S(O)₂—C₀-C₃alkyl-Y;
—C₀-C₆alkyl-aryl-heteroaryl-C₀-C₃alkyl-, wherein when the —C₀-C₃alkyl is C₁-C₃alkyl it is optionally substituted with a substituent selected from the group consisting of —N(R³)—C(O)—C₀-C₃alkyl-Y, —N(R³)—C(O)—C₀-C₃alkyl-Y, —N(R³)—C(O)—O—C₀-C₃alkyl-Y, —N(R³)—C(O)—N(R³)—C₀-C₃alkyl-Y and —N(R³)—S(O)₂—C₀-C₃alkyl-Y;
—C₀-C₆alkyl-heteroaryl-heteroaryl-C₀-C₃alkyl-, wherein when the —C₀-C₃alkyl is C₁-C₃alkyl it is optionally substituted with a substituent selected from the group consisting of —N(R³)—C(O)—C₀-C₃alkyl-Y, —N(R³)—C(O)—C₀-C₃alkyl-Y, —N(R³)—C(O)—O—C₀-C₃alkyl-Y, —N(R³)—C(O)—N(R³)—C₀-C₃alkyl-Y and —N(R³)—S(O)₂—C₀-C₃alkyl-Y;
—C₀-C₆alkyl-heteroaryl-C₀-C₃alkyl-, wherein when the —C₀-C₃alkyl is C₁-C₃alkyl it is optionally substituted with a substituent selected from the group consisting of —N(R³)—C(O)—C₀-C₃alkyl-Y, —N(R³)—C(O)—C₀-C₃alkyl-Y, —N(R³)—C(O)—O—C₀-C₃alkyl-Y, —N(R³)—C(O)—N(R³)—C₀-C₃alkyl-Y and —N(R³)—S(O)₂—C₀-C₃alkyl-Y;
—C₀-C₆alkyl-O—C(O)—N(R³)—C₀-C₃alkyl-, wherein when the —C₀-C₃alkyl is C₁-C₃ alkyl it is optionally substituted with a group selected from —C(O)—OR^{a}, —C(S)—OR^{a}, —C(O)—N(R³)—C₁-C₃alkyl, —C(S)—N(R³)—C₁-C₃alkyl, —C(O)—N(R³)(R^{3a})—, —C(S)—N(R³)(R^{3a})—, —C(O)—N(R³)—C₀-C₃alkyl-aryl, —C(S)—N(R³)—C₀-C₃alkyl-aryl, —C(O)—N(R³)—C₀-C₃alkyl-heteroaryl, —C(S)—N(R³)—C₀-C₃alkyl-heteroaryl, —C(O)—N(R³)—C₀-C₃alkyl-cycloalkyl, —C(S)—N(R³)—C₀-C₃alkyl-cycloalkyl, —C(O)-heterocyclyl, —C(S)-heterocyclyl, —C(O)—N(R³)—C₀-C₃alkyl-Y, —C(S)—N(R³)—C₀-C₃alkyl-Y, —C(O)—N(R³)-heterocyclyl, —C(S)—N(R³)-heterocyclyl, —C(O)—N(R³)—C₀-C₃alkyl-heterocycloalkyl and —C(S)—N(R³)—C₀-C₃alkyl-heterocycloalkyl;
—C₀-C₆alkyl-O—C(S)—N(R³)—C₀-C₃alkyl-, wherein when the —C₀-C₃alkyl is C₁-C₃ alkyl it is optionally substituted with a group selected from —C(O)—OR^{a}, —C(S)—OR^{a}, —C(O)—N(R³)—C₁-C₃alkyl, —C(S)—N(R³)—C₁-C₃alkyl, —C(O)—N(R³)(R^{3a})—, —C(S)—N(R³)(R^{3a})—, —C(O)—N(R³)—C₀-C₃alkyl-aryl, —C(S)—N(R³)—C₀-C₃alkyl-aryl, —C(O)—N(R³)—C₀-C₃alkyl-heteroaryl, —C(S)—N(R³)—C₀-C₃alkyl-heteroaryl, —C(O)—N(R³)—C₀-C₃alkyl-cycloalkyl, —C(S)—N(R³)—C₀-C₃alkyl-cycloalkyl, —C(O)-heterocyclyl, —C(S)-heterocyclyl, —C(O)—N(R³)—C₀-C₃alkyl-Y, —C(S)—N(R³)—C₀-C₃alkyl-Y, —C(O)—N(R³)-heterocyclyl, —C(S)—N(R³)-heterocyclyl, —C(O)—N(R³)—C₀-C₃alkyl-heterocycloalkyl and —C(S)—N(R³)—C₀-C₃alkyl-heterocycloalkyl; and
—C₁-C₃alkyl-N(R³)—C(O)—C₁-C₇alkyl-, wherein the C₁-C₃alkyl is optionally substituted with —C(O)N(R³)—C₁-C₃alkyl-Ala and the C₁-C₇alkyl is optionally substituted with a substituent selected from the group consisting of —N(R³)—C(O)—O—C₁-C₃alkyl-A^{1b}, N(R³)—C(O)—C₁-C₃alkyl-A^{1b}, —N(R³)—S(O)₂—C₁-C₃alkyl-A^{1b}, —N(R³)—S(O)₂—N(R³)—C₁-C₃alkyl-A^{1b}, —N(R³)—C(O)—N(R³)—C₁-C₃alkyl-A^{1b} and —N(R³)—S(O)₂—N(R³)—C₁-C₃alkyl-A^{1b}, wherein
A^{1a} and A^{1b} are independently selected from the group consisting of alkyl, alkenyl and a protecting group; or
A¹a and A^{1b} together via a —C₂-C₆alkylene-, —C₂-C₆alkenylene-, —C₂-C₆alkynylene- or —C₀-C₃alky-heteroaryl-C₀-C₃alky-linker, form an optionally substituted ring.

In a preferred embodiment of the present invention, L is selected from the group consisting of
—C₁-C₆alkyl-N(R³)—C₀-C₃alkyl-, wherein the C₁-C₆alkyl is optionally substituted with a substitutent selected from the group consisting of —C₁-C₄ alkyl-OR^{a}, —C₁-C₆ alkyl-N(R³)(R^{3a})—, —C₀-C₄ alkyl-C(O)OR^{a} and —C₀-C₃alkyl-C(O)—N(R³)(R^{3a})—;
—C₀-C₆alkyl-N(R³)—C(O)—C₀-C₃alkyl-, wherein the C₁-C₆alkyl is optionally substituted with a substituent selected from the group consisting of —C₁-C₄alkyl-O(R^{a})—, —C₀-C₆alkyl-C(O)O(R^{a})— and —C₁-C₆alkyl-N(R³)(R^{3a})—; and
—C₀-C₆alkyl-C(O)—N(R³)—C₀-C₃alkyl-, wherein the C₁-C₆alkyl is optionally substituted with a substituent selected from the group consisting of —C₀-C₆alkyl-O(R^{a})—, —C₀-C₆alkyl-C(O)O(R^{a})—, —C₀-C₃alkyl-C(O)—N(R³)(R^{3a}) and —C₀-C₆alkyl-N(R³)(R^{3a})—.

In a preferred embodiment of the present invention, L is selected from the group consisting of
—C₀-C₆alkyl-N(R³)—C(O)—C₁-C₇alkyl-, wherein the C₁-C₇alkyl is optionally substituted with a substituent selected from the group consisting of —N(R⁷)(R^{7a}), —N(R³)C(O)—C₀-C₃alkyl-heterocyclyl, —N(R³)—C(O)—C₀-C₆alkylaryl-R^{a}, —N(R³)—C(O)—C₁-C₆alkyl-R^{a} and —N(R³)—C(O)—O—C₀-C₃alkyl-Y wherein heterocyclyl is optionally substituted;
—C₀-C₆alkyl-C(O)—N(R³)—C₁-C₇alkyl-, wherein the C₁-C₆alkyl is optionally substituted with —N(R⁷)(R^{7a});
—C₀-C₆alkyl-C(O)—N(R³)—C₁-C₇alkyl-, wherein the C₁-C₇alkyl is optionally substituted with a substituent selected from the group consisting of aryl-aryl, aryl-heteroaryl, heteroaryl-heteroaryl, heteroaryl-aryl and heteroaryl; and
—C₁-C₃alkyl-N(R³)—C(O)—C₁-C₇ alkyl-, wherein the C₁-C₃alkyl is optionally substituted with —C(O)N(R³)—C₁-C₃alkyl-Ala and the C₁-C₇alkyl is optionally substituted with a substituent selected from the group consisting of —N(R³)—C(O)O—C₁-C₃alkyl-A^{1b}, —N(R³)—C(O)—C₁-C₃alkyl-A^{1b}, —N(R³)—S(O)₂—C₁-C₃alkyl-A^{1b}, —N(R³)—C(O)—N(R³)—C₁-C₃alkyl-A^{1b} and —N(R³)—S(O)₂—N(R³)—C₁-C₃alkyl-A^{1b}, wherein
A₁ₐ and A_{1b} are independently selected from the group consisting of alkyl, alkenyl and a protecting group; or
A₁ₐ and A_{1b} together via a —C₂-C₆alkylene, —C₂-C₆alkenylene, —C₂-C₆alkynylene, —C₀-C₃alkyl-heteroaryl-C₀-C₃alkyl-linker or —C₀-C₃alkyl-aryl-C₀-C₃alkyl-linker, form an optionally substituted ring, and

In a preferred embodiment of the present invention, L is a selected from the group consisting of
—C₀-C₇alkyl-N(R₃)—C(O)-heterocyclyl-C₀-C₆alkyl-, wherein a C₁-C₇alkyl is optionally substituted with —C₀-C₃alkyl-C(O)ORₐ or —C₁-C₃alkyl-ORₐ; and
—C₀-C₇alkyl-O—C(O)-heterocyclyl-C₀-C₆alkyl-, wherein a C₁-C₇alkyl is optionally substituted with —C₀-C₃alkyl-C(O)ORₐ or —C₀-C₃alkyl-ORₐ.

In a preferred embodiment of the present invention, L is selected from the group consisting of a covalent bond, —(CH₂)₁₋₄—, —(CH₂)₀₋₄—(CR³═CR³)—(CH₂)₀₋₄—, —(CH₂)₀₋₄—(C≡C)—(CH₂)₀₋₄—, —(CH₂)₀₋₃ N(R³)C(O)—, —(CH₂)₀₋₃—C(O)N(R³)—, —(CH₂)₀₋₃ N(R³)C(O)—(CR³═CR³)—, —(CH₂)₀₋₃—N(R³)—(CH₂)₂₋₄ N(R³)C(O)—, —(CH₂)₀₋₃—O—(CH₂)₂₋₄—N(R³)C(O)—, —(CH₂)₀₋₃C(O)—(CH₂)₀₋₃—, —(CH₂)₀₋₃—(CR^{a}═CR^{a})—C(O)—(CH₂)₀₋₃—, —(CH₂)₀₋₃C(O)—(CR^{a}═CR^{a})—(CH₂)₀₋₃—, —C₀-C₆alkyl-N(R³)—C(O)-heterocyclyl-C₀-C₃alkyl-, —C₀-C₆alkyl-S(O)₂-heterocyclyl-C₀-C₃alkyl-, —(CH₂)₀₋₃—S(O)₂—N(R³)—(CH₂)₀₋₃—, —(CH₂)₀₋₃ N(R³)—S(O)₂—(CH₂)₀₋₃—, —(CH₂)₀₋₃N(R³)—(CH₂)₀₋₃—, —(CH₂)₀₋₃N(R³)—(CH₂)₁₋₃—(CR^{a}═CR^{a})—, —(CH₂)₀₋₃C═N—O—(CH₂)₀₋₃—, —(CH₂)₀₋₃N(R⁷)—(CH₂)₀₋₃—, —(CH₂)₀₋₃S—(CH₂)₀₋₃—, —(CH₂)₀₋₃O—(CH₂)₀₋₃—, —(CH₂)₀₋₃S(O)—(CH₂)₀₋₃—, —(CH₂)₀₋₃S(O)₂—(CH₂)₀₋₃—, —(CH₂)₀₋₃CH═CH—(CH₂)₂₋₃—, —(CH₂)₀₋₃N(R³)—C(O)—N(R³)—(CH₂)₀₋₃, —(CH₂)₀₋₃N(R³)—C(O)—O—(CH₂)₀₋₃, —(CH₂)₀₋₃O—C(O)—N(R³)—(CH₂)₀₋₃—, —(CH₂)₀₋₃ N(R³)—C(O)—N(R₃)—S(O)₂—(CH₂)₀₋₃—, and —(CH₂)₀₋₃N(R³)—C(O)—N(R³)—C(O)—(CH₂)₀₋₃—.

In a preferred embodiment of the present invention, B₁, B₂ and B₃ are independently selected from the group consisting of D-Gly, L-Gly, D-Pro, L-Pro, D-Tyr, L-Tyr, D-Tyr(ORₐ), L-Tyr(ORₐ), D-Phe, L-Phe, D-PheR₄, L-PheR₄, D-Aib, L-Aib, D-Ala, L-Ala, D-ProR₃, L-ProR₃, D-Ile, L-Ile, D-Leu, L-Leu D-PheR₃, L-PheR₃, D-Pip and L-Pip.

In a preferred embodiment of the present invention, each alkyl, alkenyl, alkynyl, heteroalkyl, benzyl and heterocyclyl moiety of R⁷ and R^{7a} is independently optionally substituted with one or more substituents selected from the group consisting of oxo, —OH, —CN, C₁-C₆alkyl, C₁-C₆alkoxy, —NO₂, —N(R³)(R^{3a}), halo, —SH and mono- to per-halogenated C₁-C₆alkyl.

In a preferred embodiment of the present invention, Y is selected from the group consisting of aromatic polycycle, non-aromatic polycycle, mixed aryl and non-aryl polycycle, polyheteroaryl, non-aromatic polyheterocycle, mixed aryl and non-aryl polyheterocycle, each of which is optionally substituted.

In a preferred embodiment of the present invention, Y is selected from the group consisting of aryl, aryl-aryl, heteroaryl, aryl-heteroaryl, heteroaryl-aryl, cycloalkyl, heterocyclyl and heterocyclyl-heteroaryl, each of which is optionally substituted.

In a preferred embodiment of the present invention, R^{a}, R^{b} and R^{c} are independently selected from the group consisting of —H, C₁-C₃alkyl, C₃-C₆cycloalkyl, aryl, heteroaryl, and aryl-C₁-C₃alkyl-.

In a preferred embodiment of the present invention, R^{a} and R^{b} together with the nitrogen atom to which they are attached form a 3 to 9-membered heterocyclyl, heteroaryl, or heterocyclyl-aryl, wherein each of the heterocyclyl, heteroaryl and heterocyclyl-aryl is optionally substituted.

In a preferred embodiment of the present invention, R³ and R^{3a} are independently selected from the group consisting of —H, OH, C₁-C₆alkyl, C₃-C₆cycloalkyl, —C(O)CF₃, —C(O)H, —C₁-C₄alkyl-C(O)OR^{a}, heterocyclyl, —C₂-C₄alkyl-OR^{a}, C₂-C₄alkylene; C₂-C₆alkenyl, C₂-C₆ hydroxyalkyl —C₁-C₆ alkylaryl, aryl, —C₀-C₆alkylheteroaryl, and —C₁-C₃alkyl-C(O)NR^{a}-heteroaryl.

In a preferred embodiment of the present invention, R³ and R^{3a} are independently selected from the group consisting of —C₁-C₆alkylaryl, t-butyl, benzyl and aryl.

In a preferred embodiment of the present invention, R³ and R^{3a} are independently selected from the group consisting of ethanol, tetrahydro-2H-pyran, phenyl and benzyl.

In a preferred embodiment of the present invention, R³ and R^{3a} are independently C₁-C₄ alkyl.

In a preferred embodiment of the present invention, in a —N(R³)(R^{3a}) group, the R³ and the R^{3a} together with the nitrogen atom to which they are attached optionally form a ring selected from the group consisting of morpholinyl, piperazinyl, piperidinyl, pyrrolydinyl, and azetidinyl.

In a preferred embodiment of the present invention, R⁴ is selected from the group consisting of —H, —CH₃, —S(O)₂—N(R³)(R^{3a}), —SO₃H, —O—C₂-C₄alkyl-heterocyclyl, —O—C₀-C₄alkyl-aryl, —O—C₀-C₄-alkyl-heteroaryl, —O—C(O)N(R³)—C₀-C₄alkyl-aryl, —O—C(O)N(R³)—C₀-C₄alkyl-heteroaryl, —O—C₀-C₄alkyl-heterocyclyl-aryl, —O—C₀-C₄alkyl-heterocyclyl-heteroaryl, —N(R³)—C₂-C₄alkyl-heterocyclyl, —(CH₂)₀₋₄OR^{a}, —(CH₂)₀₋₄N(R³)(R^{3a}), —F, —Cl, —Br, —CF₃, —CN, —CH₂OH, —OH, —OCH₃, —NO₂, Ph, aryl, heteroaryl, —N(R³)C(O)CH₂R³, —N(R³)SO₂CH₂R^{a}, —O(CH₂)₂₋₄N(R³)(R^{3a}), —SR^{a}, —S(O)CH₂R³, —SO₂CH₂R^{a}, —(CH₂)₀₋₄C(O)OR^{a}, —CH═CHC(O)OR^{a}, —CH═CHC(O)N(R³)(R^{3a}), —N(R³)C(O)CF₃ and —N(R³)(CH₂)₂N(R³)(R^{3a}).

In a preferred embodiment of the present invention, L is selected from the group consisting of 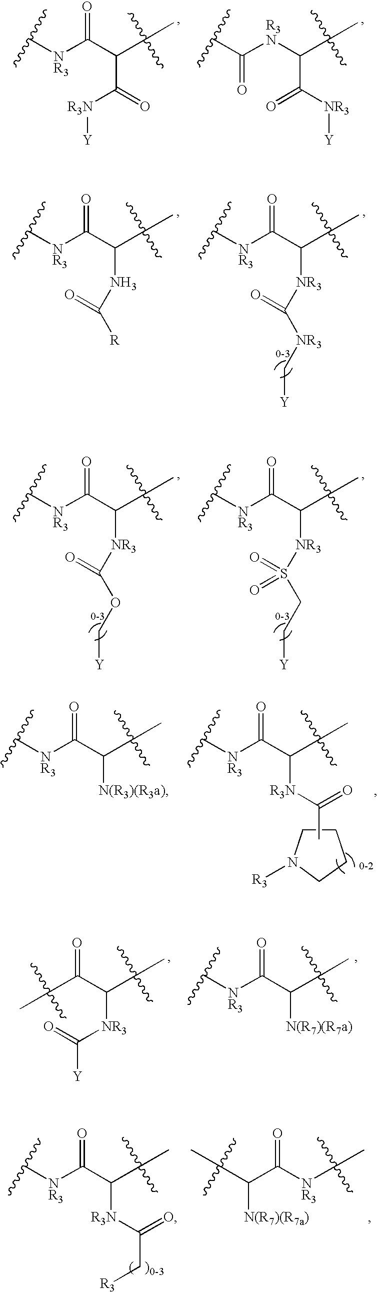 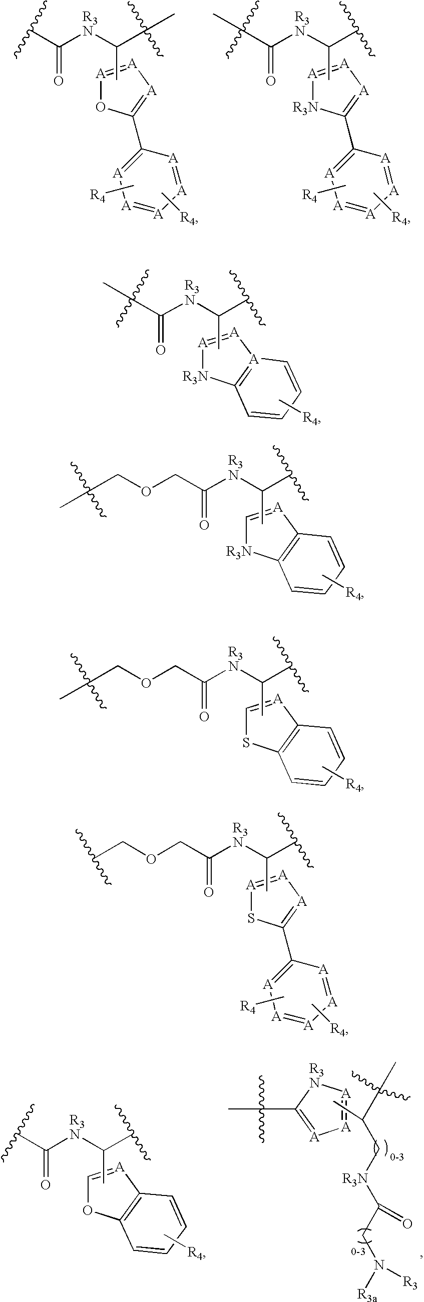 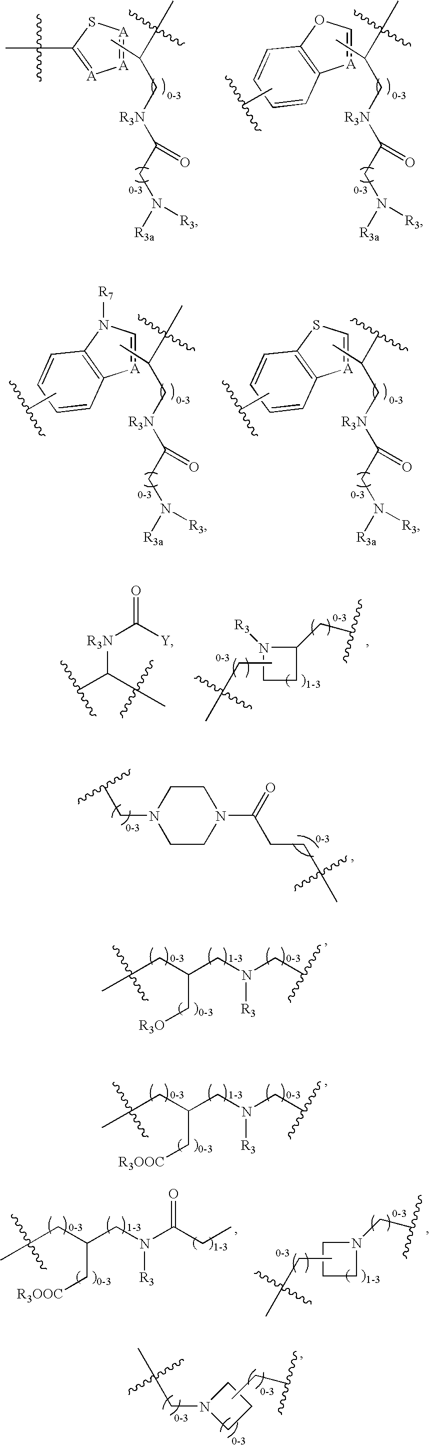 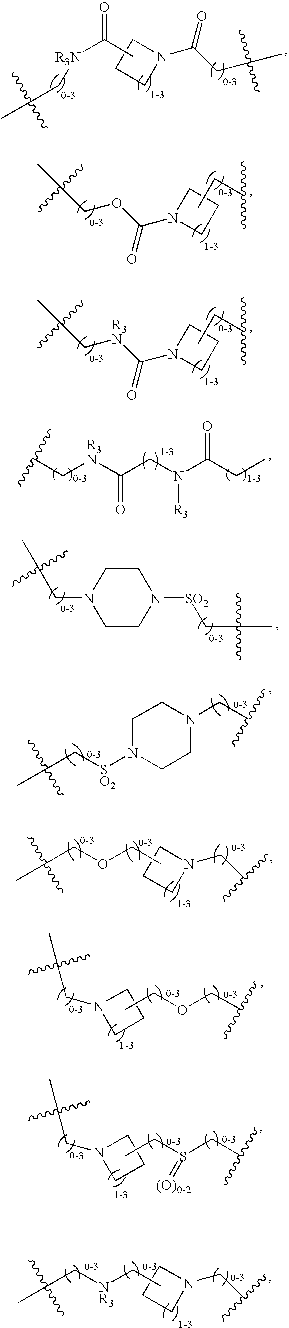 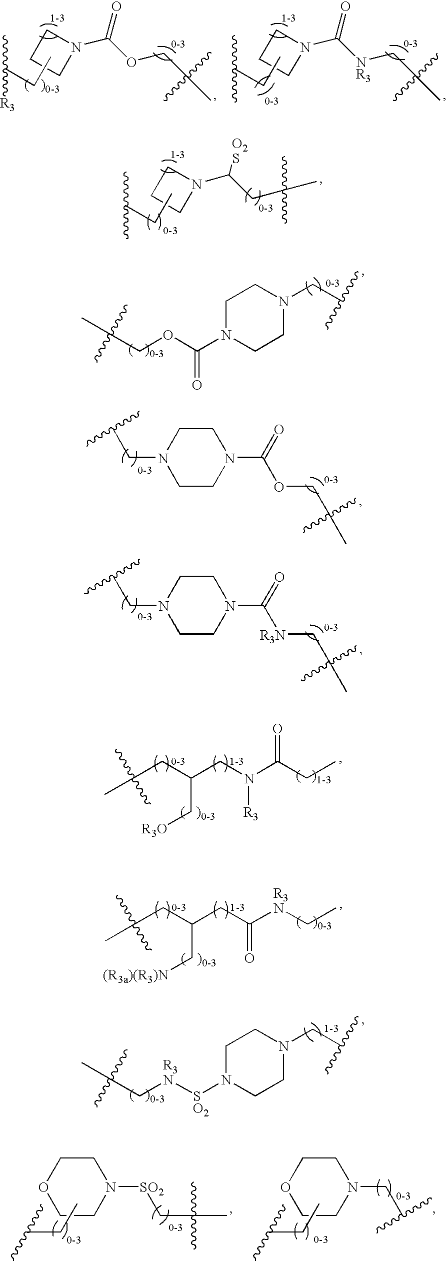 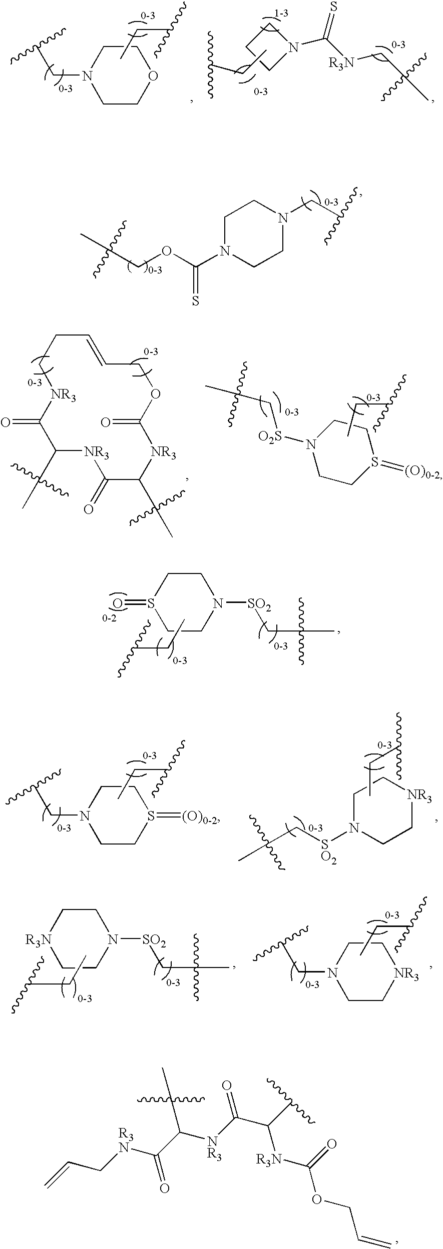 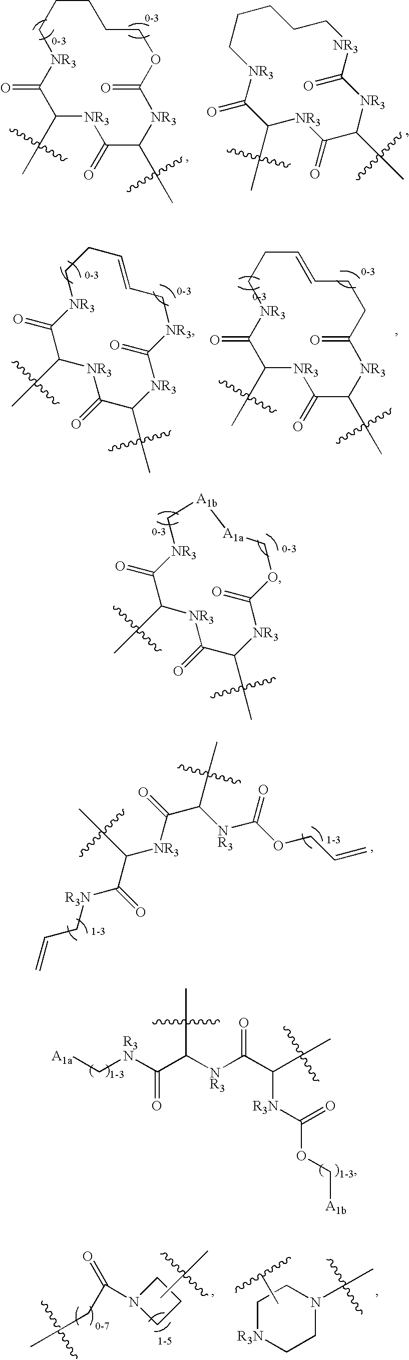 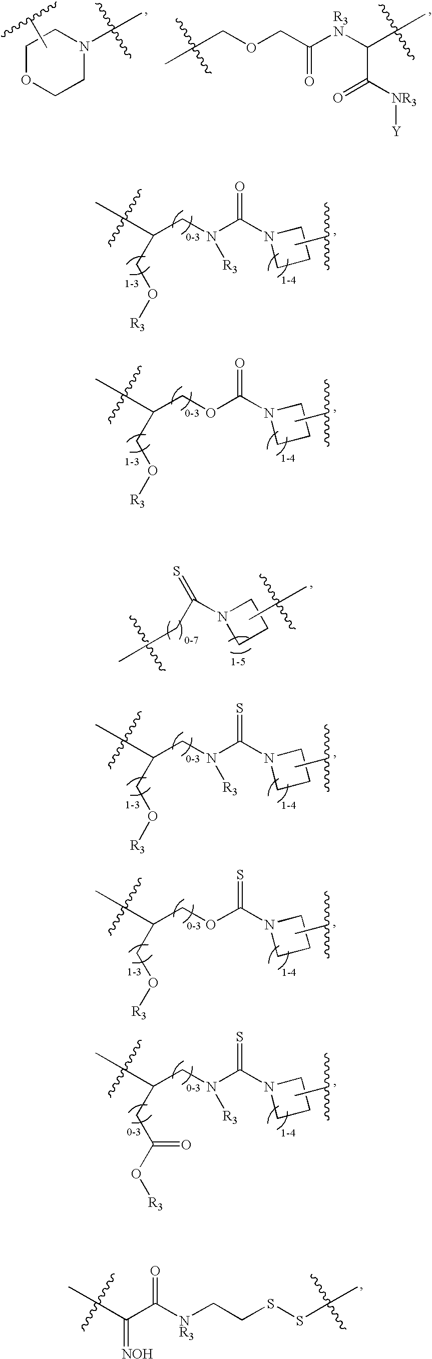 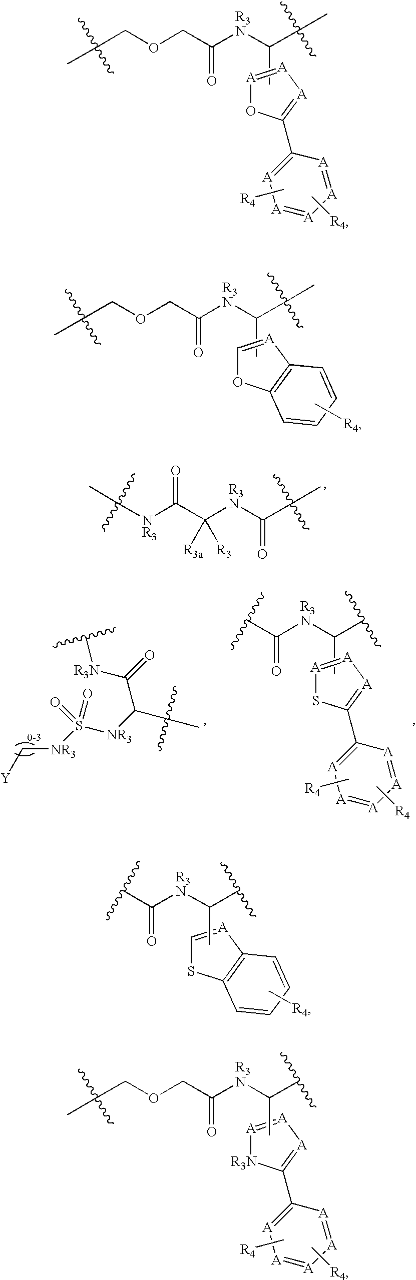 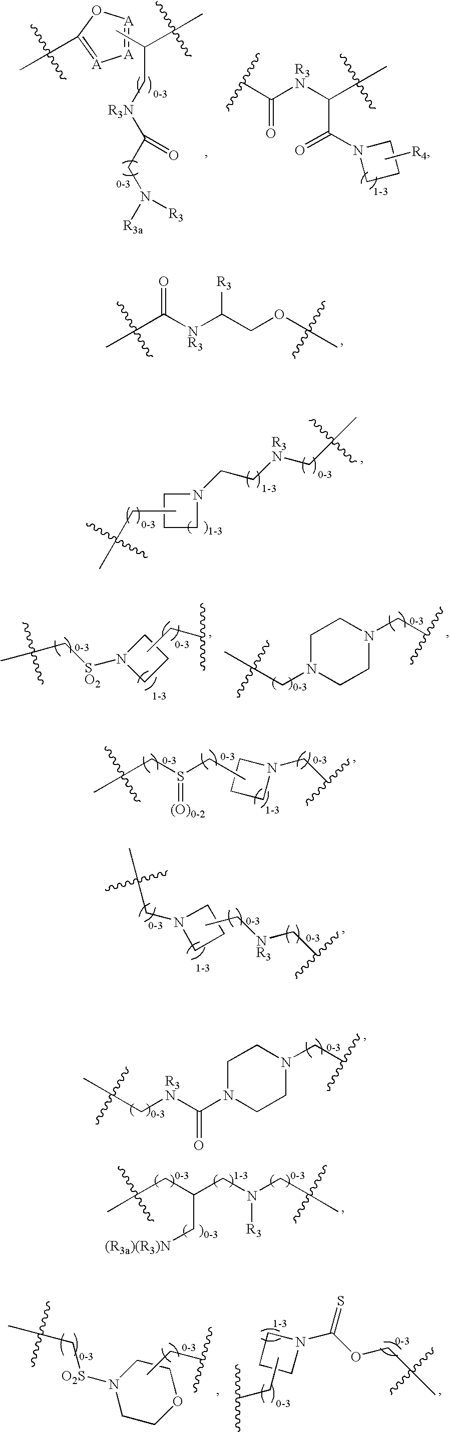 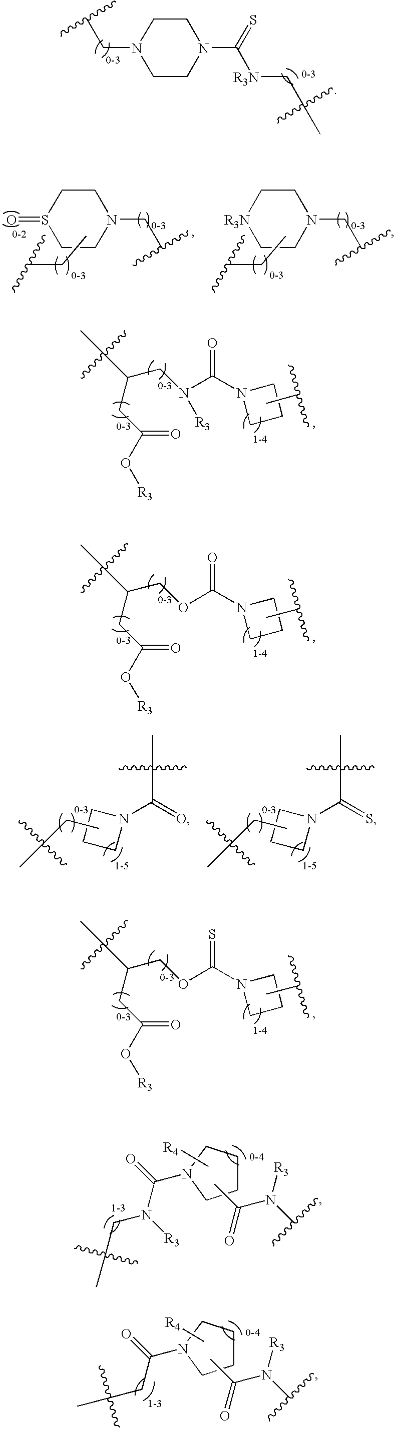 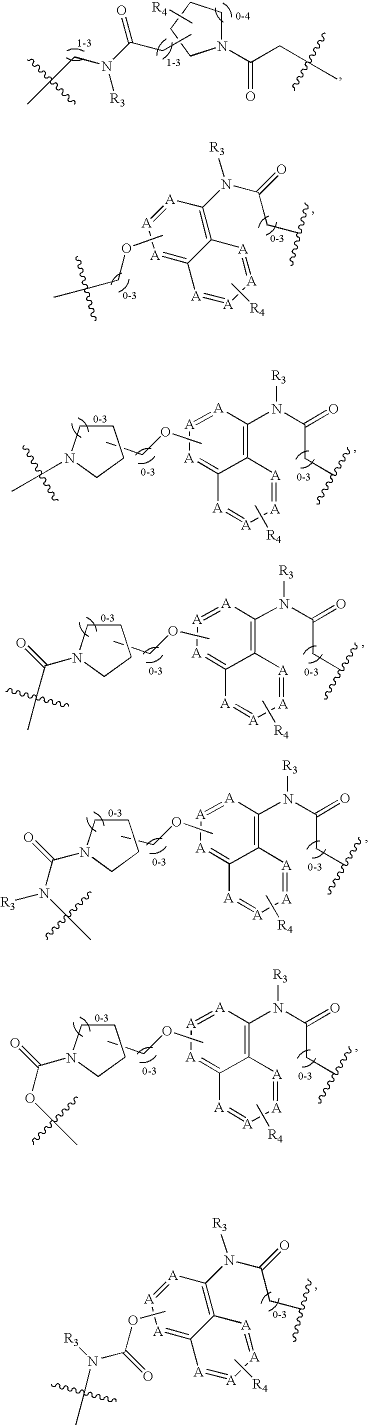 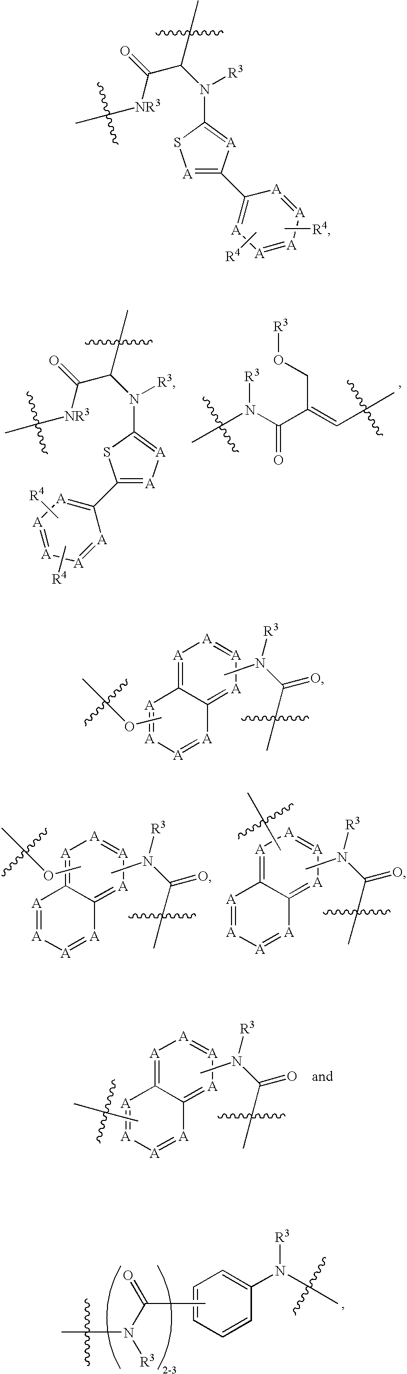
wherein
A₁ₐ and A_{1b} are independently selected from the group consisting of alkyl, alkenyl and protecting group, and
A is N, CH or C (when A is attached to Y or Z), wherein there may be 0, 1, 2 or 3 nitrogen.

In a preferred embodiment of the present invention, Z is selected from the group consisting of 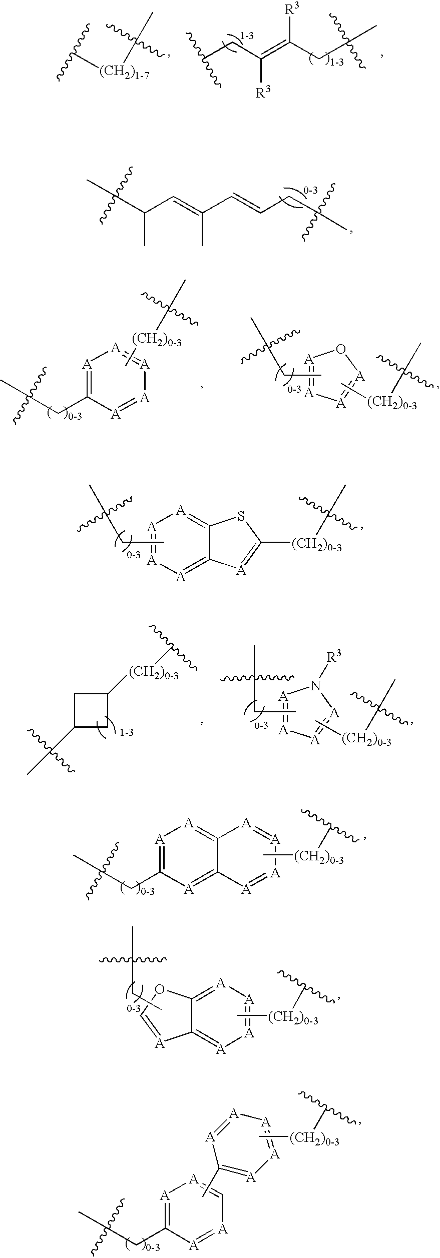 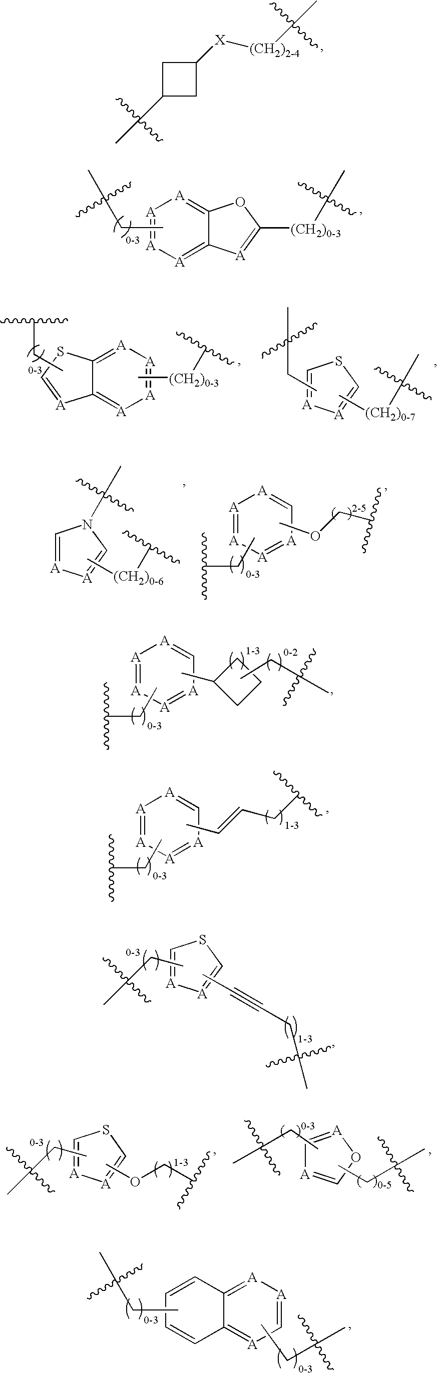 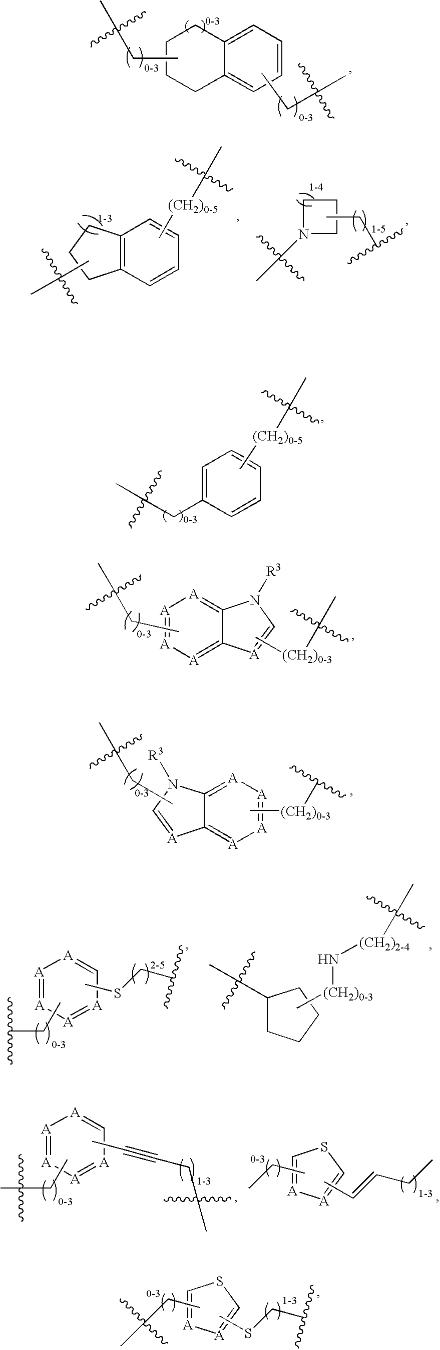 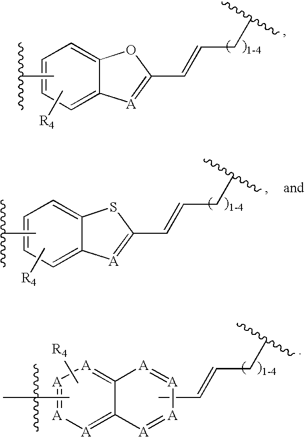
wherein A is nitrogen, —CH═ or —C(R⁴)═, wherein there may be 0, 1, 2 or 3 nitrogen.

In a preferred embodiment of the present invention, Y is selected from the group consisting of 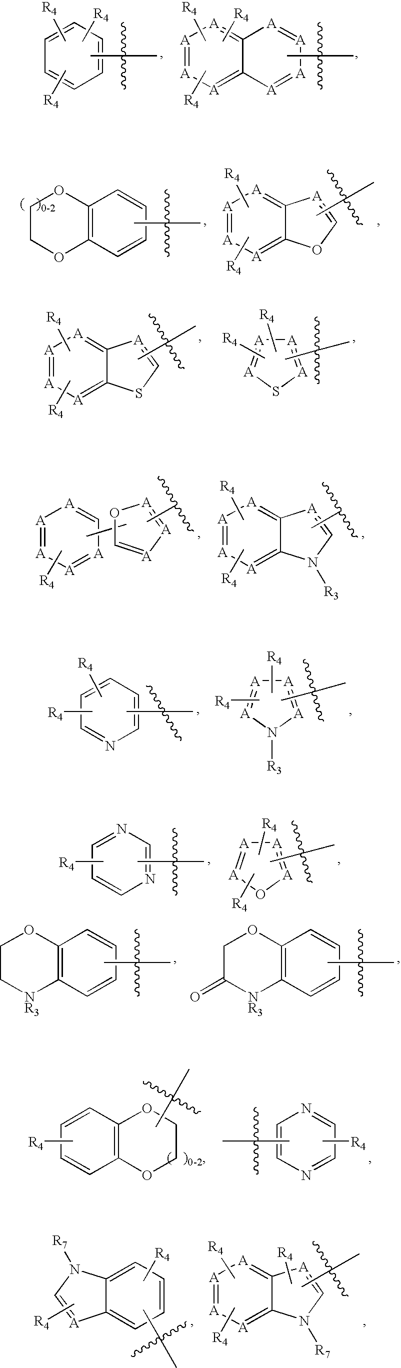 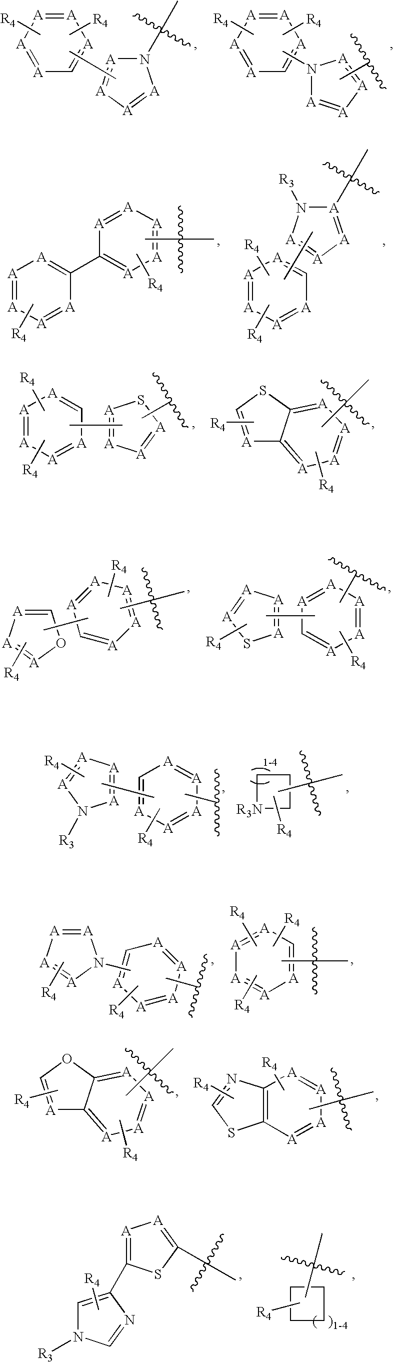 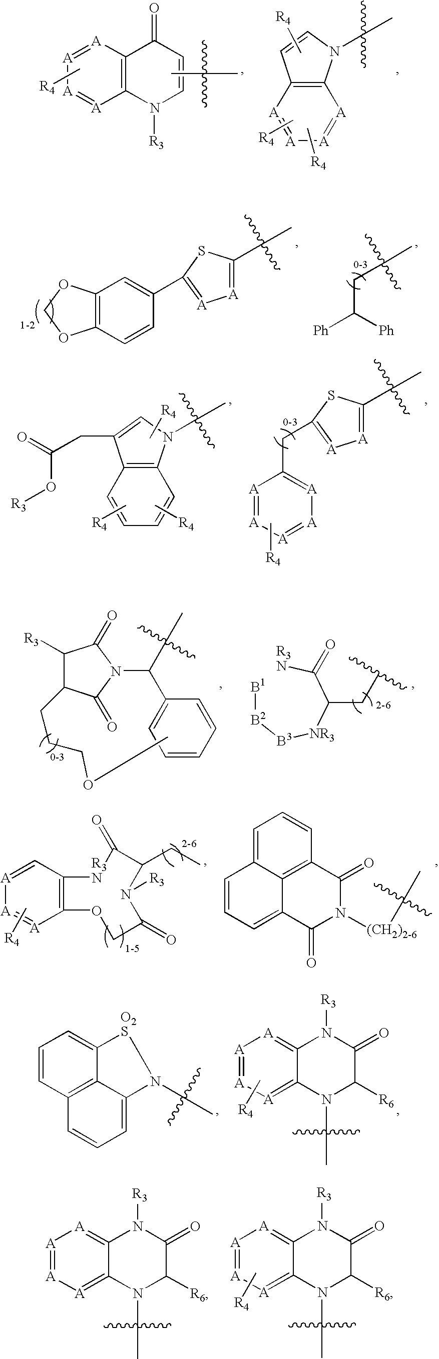 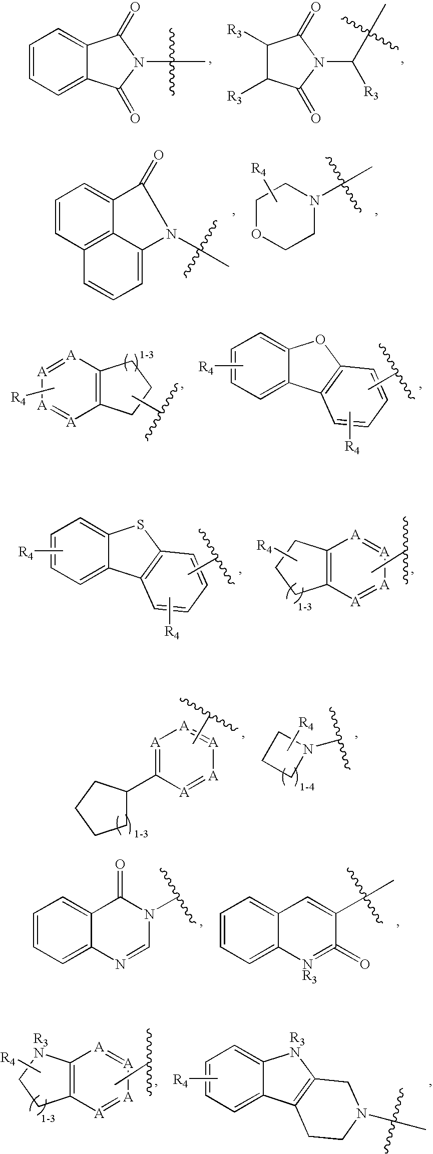 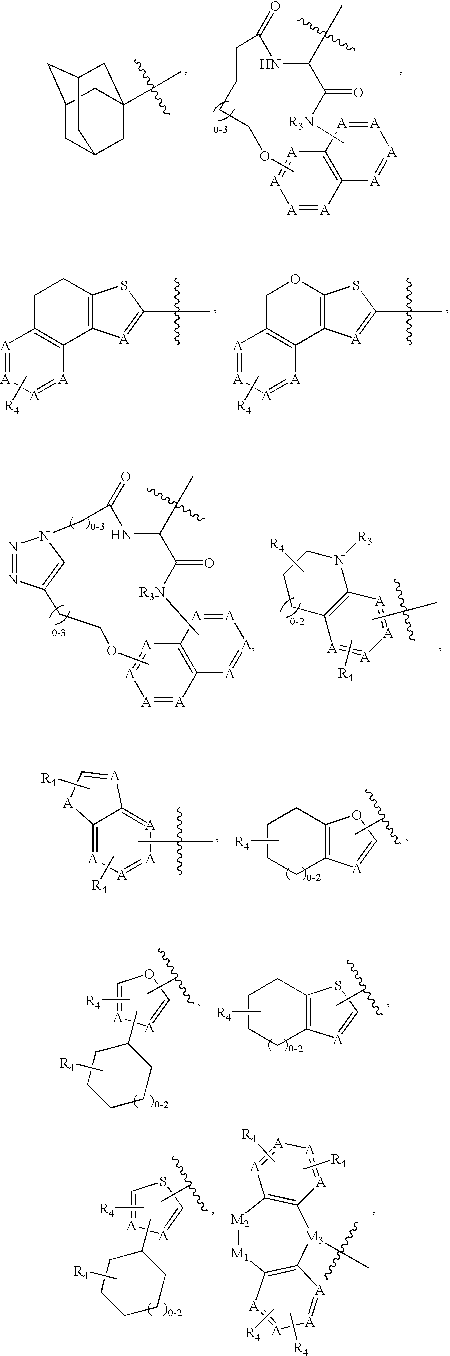 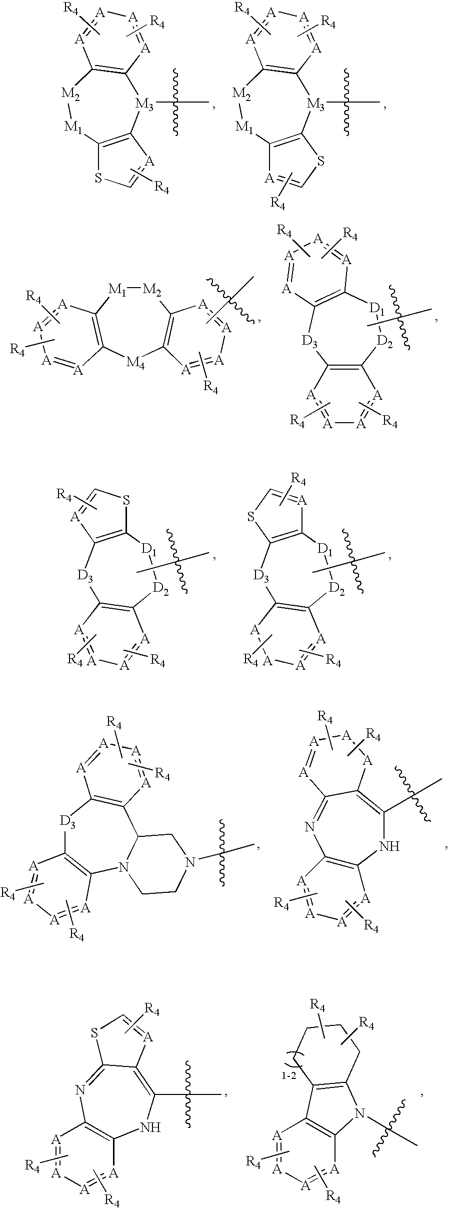 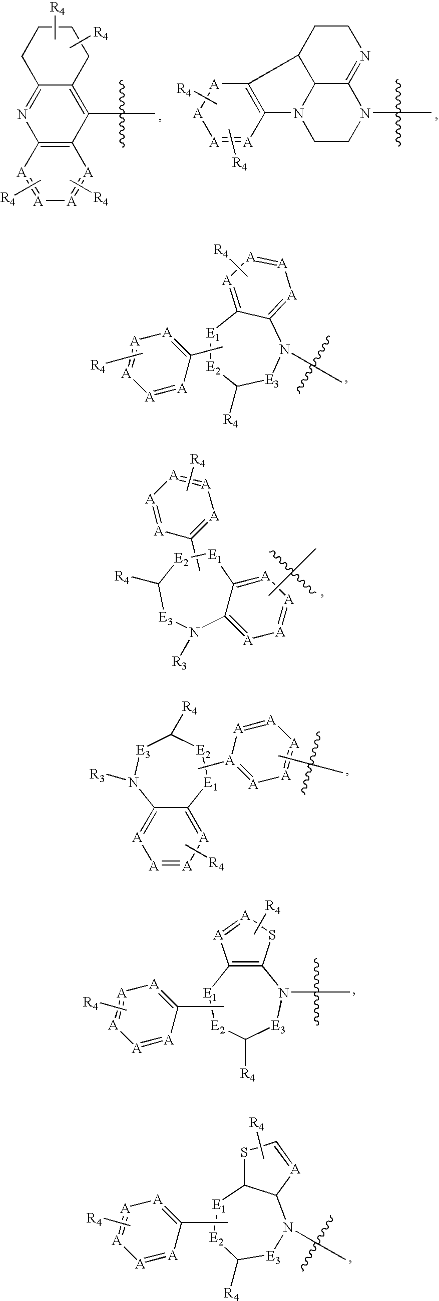 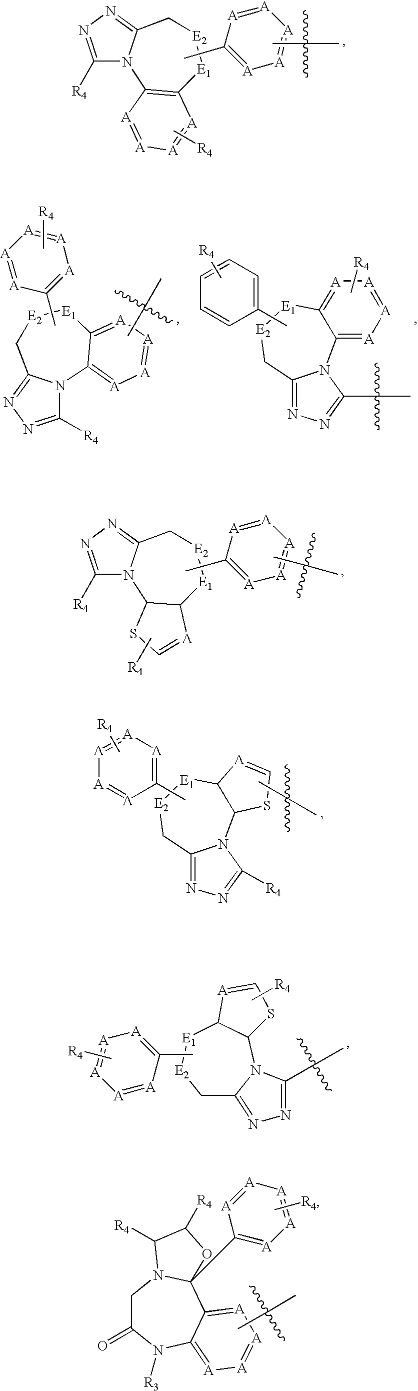 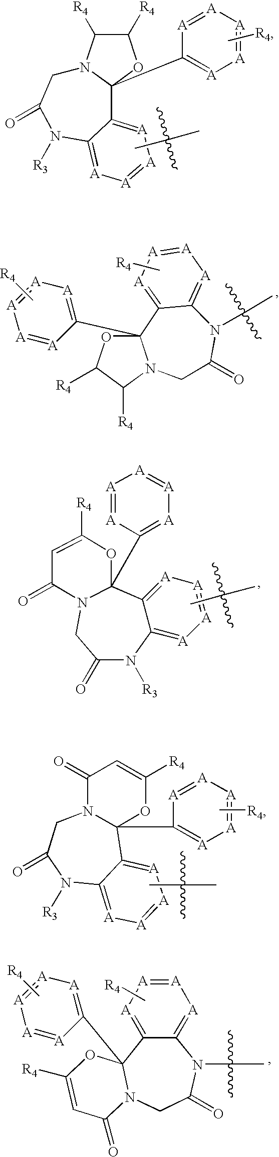 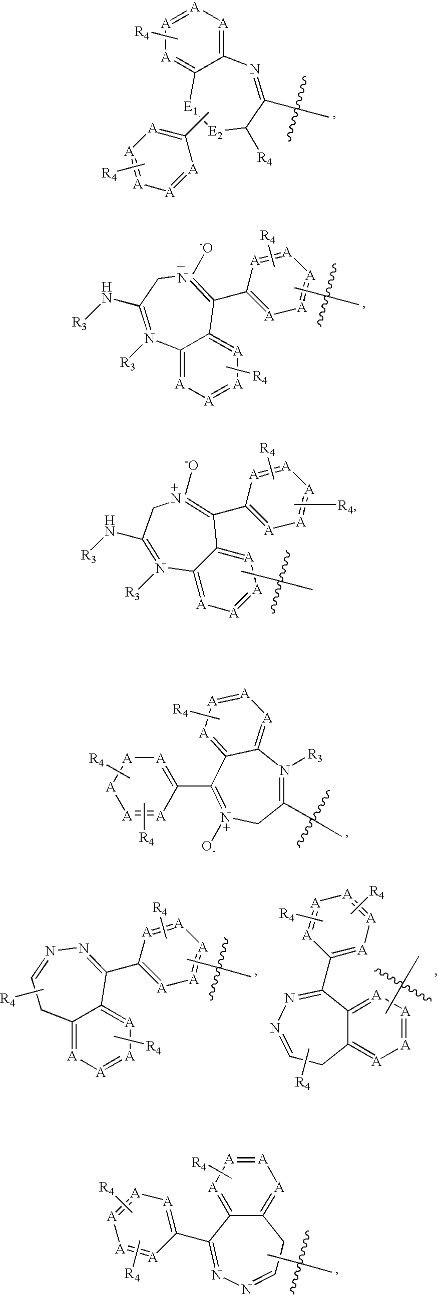 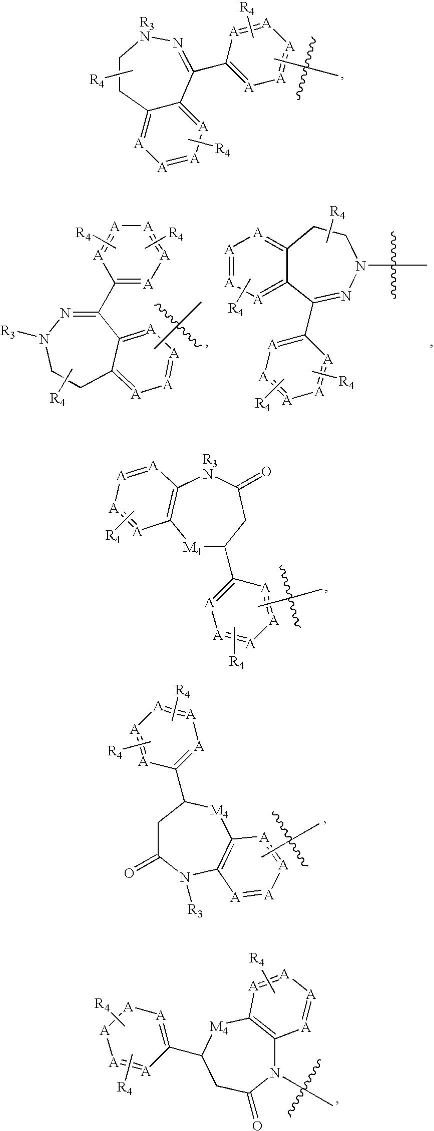 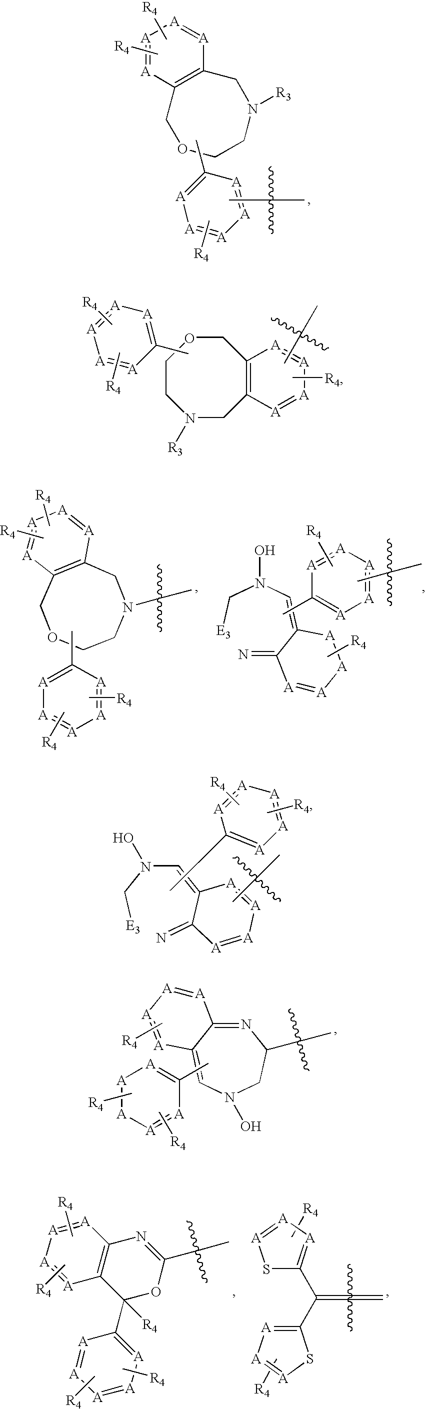 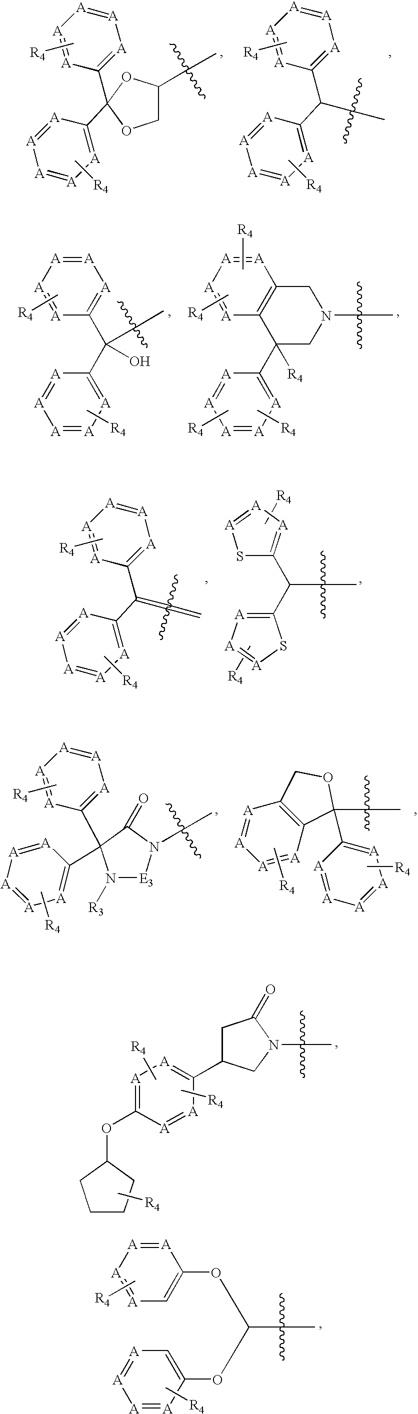 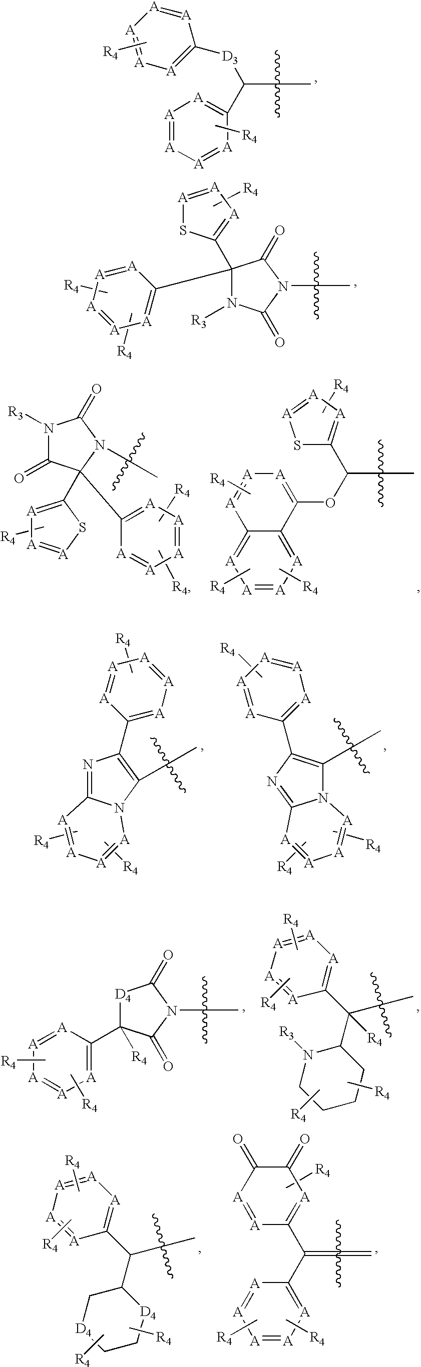 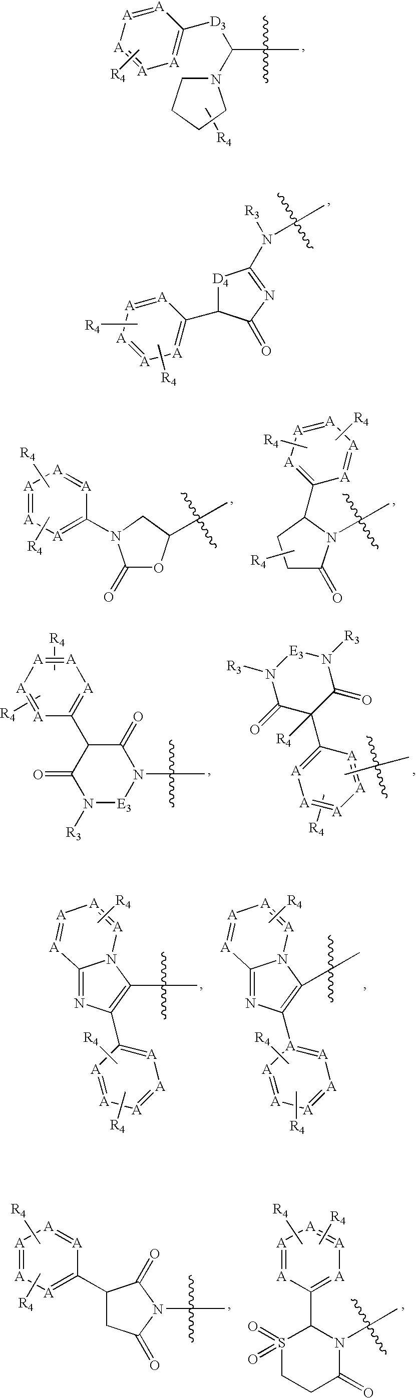 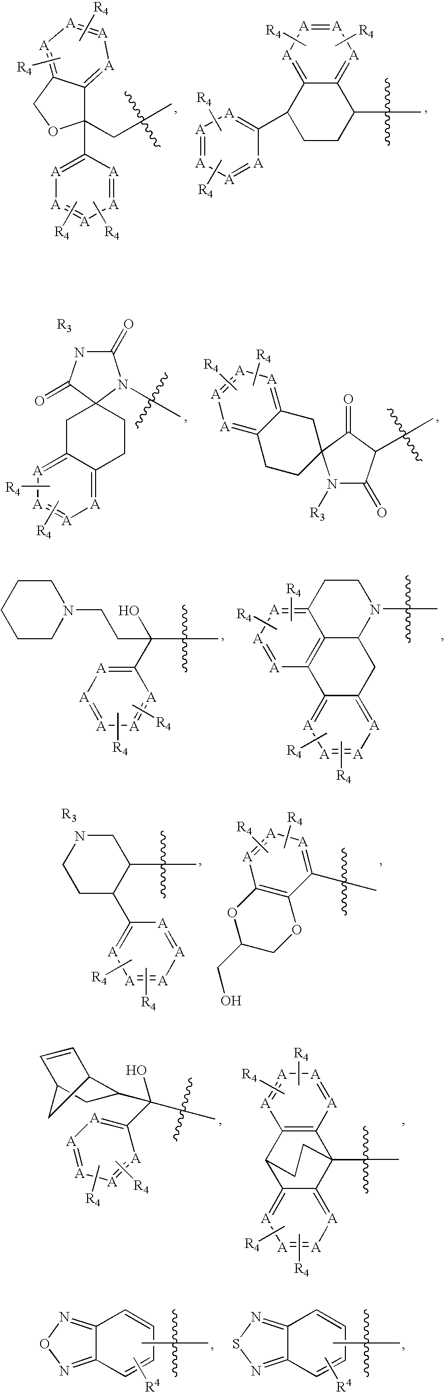 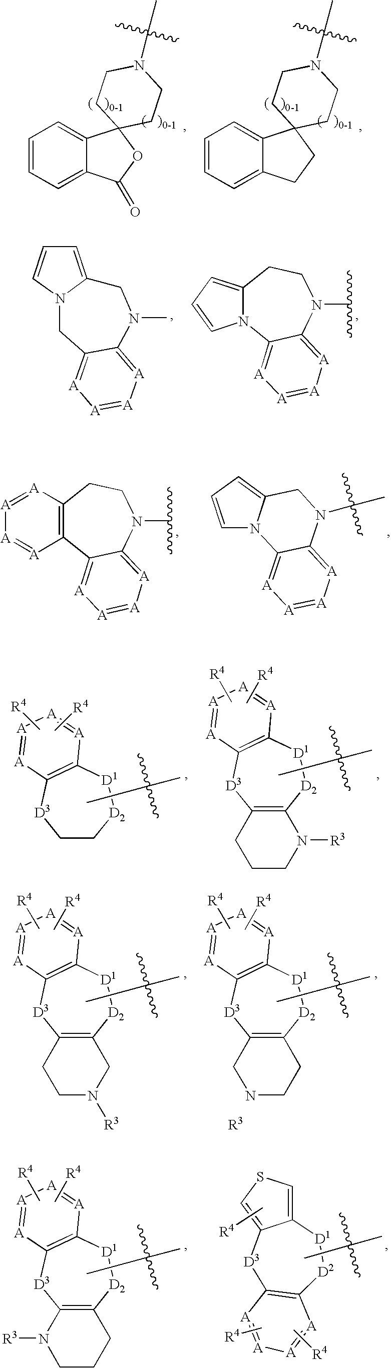 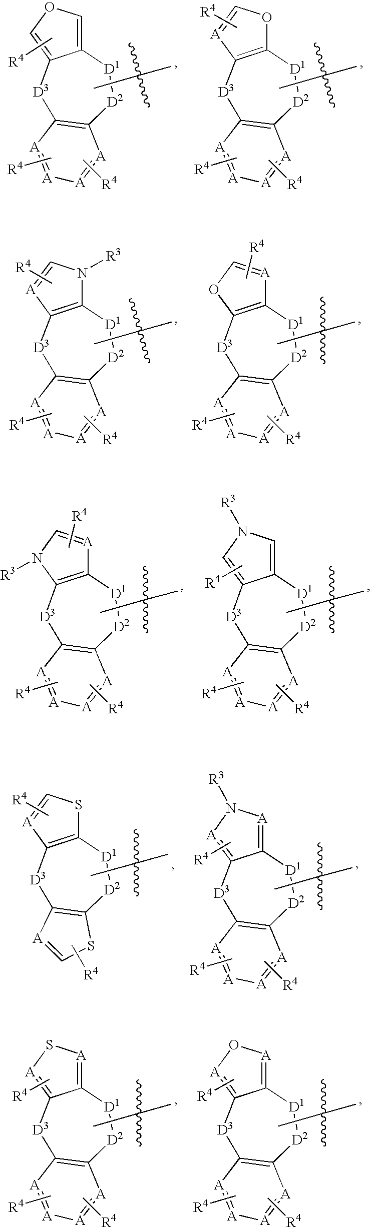 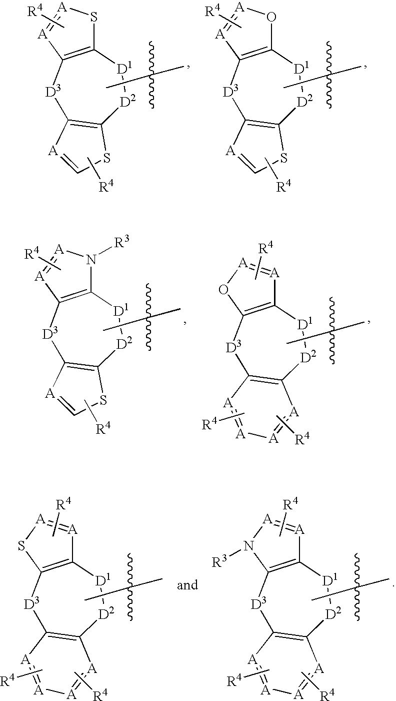
wherein
A is nitrogen, —C(H)═ or —C(R⁴)=5 wherein there may be 0, 1, 2 or 3 nitrogen;
B¹ 5B and B³ are each independently a natural or synthetic amino acid;
M₁-M₂ is selected from the group consisting of a covalent bond, —N(R³)CH₂—, —CH₂N(R³)—, —S(O)₀₋₂—CH₂—, —CH₂S(O)₀₋₂—, —O—CH₂—, —CH₂—O—, —C(O)N(R³)—, —N(R³)C(O)—, —SO₂N(R³)—, —N(R³)SO₂—, —CH(R^{a})CH₂—, —CH₂CH(R^{a})—, —N═C(R^{a})—, —C(R^{a})═N—, —CH₂—CH₂—, —CH═CH—, —CH(R^{a})—CH(R^{a})—, —C(R^{a})═C(R^{a})—, —CH₂—, —C(R^{a})(R^{a})—, —S—, —N(R³) and absent;
M₃ is selected from the group consisting of 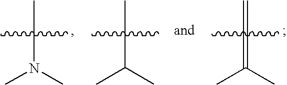
M₄ is selected from the group consisting of 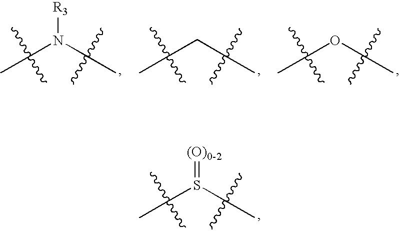
   and covalent bond;
   wherein, when M₁-M₂ is covalent bond, M₄ is 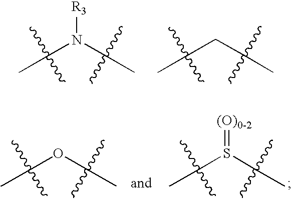
D₁-D₂ is selected from the group consisting of a 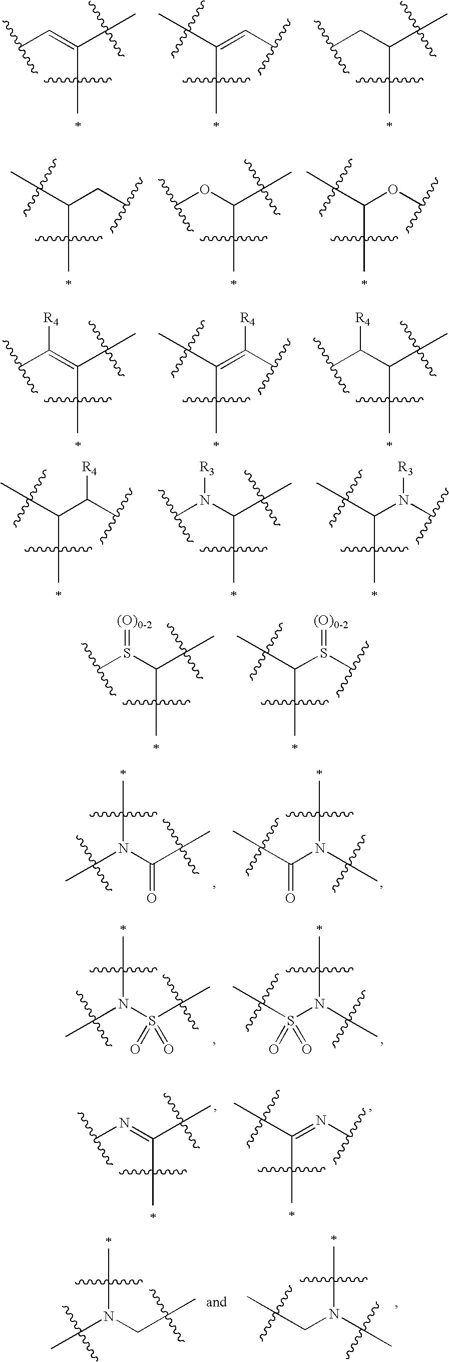
   wherein, * represents the point of attachment to Q;
D₃ is selected from the group consisting of a covalent bond, 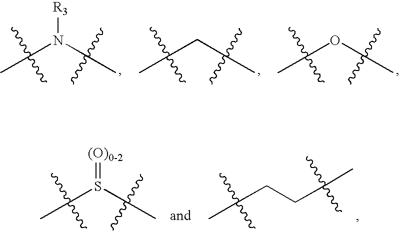
   wherein the 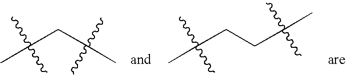
   optionally substituted
D₄ is selected from the group consisting of 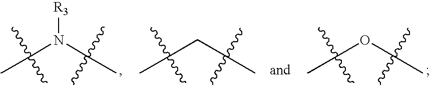
   wherein the 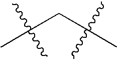
   is optionally substituted,
E₁-E₂ is selected from the group consisting of 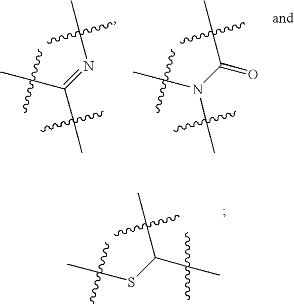
E₃ is selected from the group consisting of —C(O)—, —C(S)—, —CH₂—, —C(OH)₂— and —C═NR₃—; and
R⁶ is selected from the group consisting of —H, —C₁-C₆alkyl, —C₂-C₆alkenyl, —C₂-C₆alkynyl, —C₁-C₆heteroalkyl, heterocyclyl-C₀-C₆alkyl-, aryl-C₀-C₆alkyl-, heteroaryl-C₀-C₆alkyl-, C₃-C₆cycloalkyl-C₀-C₆alkyl-, N(R³)(R^{3a})—C₁-C₆alkyl-, N(R³)(R^{3a})—C(O)—C₁-C₆alkyl- and N(R³)(R^{3a})—C(S)—C₁-C₆alkyl-, wherein each alkyl, alkenyl, alkynyl, heteroalkyl, cycloalkyl, aryl, heteroaryl, or heterocyclyl moiety is optionally substituted.

In a preferred embodiment of the present invention, each alkyl, alkenyl, alkynyl, heteroalkyl, cycloalkyl, aryl, heteroaryl, and heterocyclyl moiety of R⁶ is independently optionally substituted with one or more groups independently selected from R⁴.

In a preferred embodiment of the present invention R⁶ is selected from the group consisting of

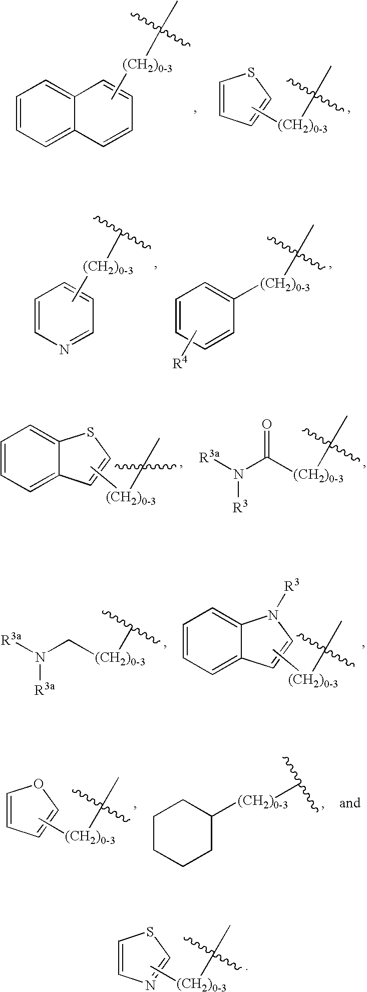

In a preferred embodiment according to the present invention, R⁷ is selected from the group consisting of —H, optionally substituted C₁-C₆ alkyl, —(CH₂)₂₋₄OR^{a}, —OMe, —(CH₂)₂₋₄N(R³)(R^{3a}), —C(O)Ot-butyl, —C(O)O-benzyl, —(CH₂)₂-morpholinyl and —(CH₂)₂-piperazynnyl.

In a preferred embodiment according to the present invention,

W and M are nitrogen;

R^{a}, R^{b} and R^{c} are —H;

Z is —C₁-C₈ alkyl-;

L is covalent bond, —C₀-C₆ alkyl-N(R³)C(O)—C₀-C₃ alkyl or —C₀-C₆ alkyl-C(O)N(R³)—C₀-C₃ alkyl, preferably —C₀-C₆ alkyl-N(R³)C(O)—C₀-C₃ alkyl; and

Y is selected from the group consisting of aryl, heteroaryl, aryl-aryl, heteroaryl-aryl-, aryl-heteroaryl- and polycycle, wherein each alkyl, aryl, heteroaryl and polycycle group is optionally substituted. Preferably, the alkyl, aryl, heteroaryl and polycycle groups are optionally substituted with aryl-C₀-C₆alkyl-O—, heteroaryl-C₀-C₆alkyl-O—, heteroaryl-O— or aryl-, said aryl-C₀-C₆alkyl-O—, heteroaryl-C₀-C₆alkyl-O—, heteroaryl-O— or aryl-groups being further optionally substituted, preferably with halo.

In a preferred embodiment according to the present invention, the compounds are represented by the formula (TI): 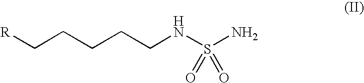
or an N-oxide, hydrate, solvate, pharmaceutically acceptable salt, prodrug or complex thereof, wherein R is selected from the group consisting of:

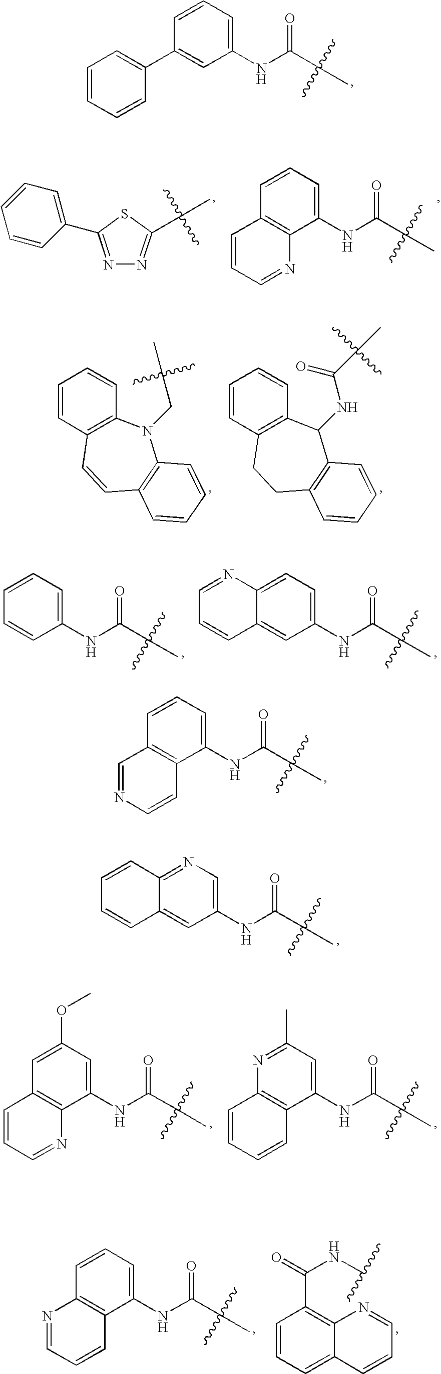

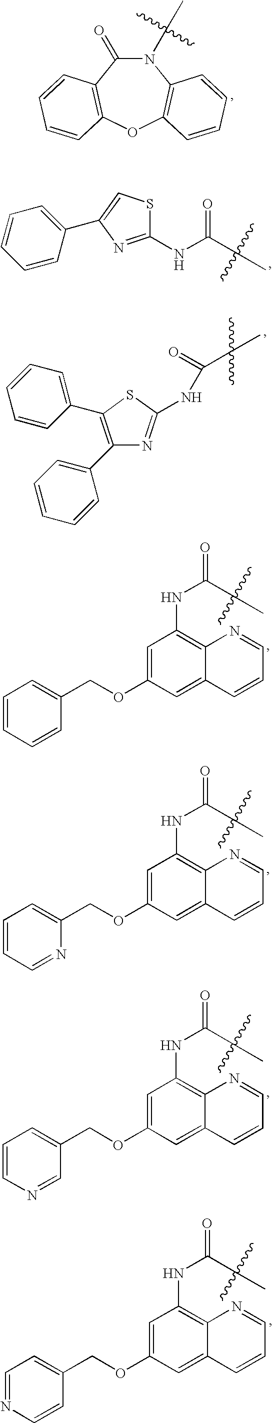

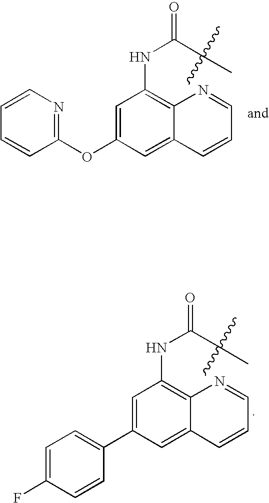

In a preferred embodiment according to the present invention,

W is nitrogen or oxygen;

M is nitrogen;

R^{a}, R^{b} and R^{c} are —H;

Z is —C₁-C₈ alkyl- or —C₁-C₈ alkyl-C(O)—;

L is —C₀-C₆ alkyl-N(R³)C(O)—C₀-C₃ alkyl; and

Y is aryl, heteroaryl, heteroaryl-aryl or aryl-heteroaryl, wherein the alkyl, aryl and heteroaryl groups are optionally substituted. Preferably, the alkyl, aryl and heteroaryl groups are optionally substituted with a substituent selected from the group consisting of alkoxy, alkyl, aryl, —O-alkyl-heteroaryl and —O-alkyl-aryl.

In a preferred embodiment according to the present invention, the compounds are represented by the formula (III): 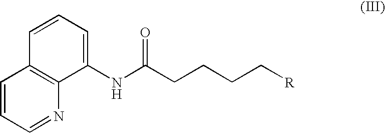
or an N-oxide, hydrate, solvate, pharmaceutically acceptable salt, prodrug or complex thereof, wherein R is selected from the group consisting of:

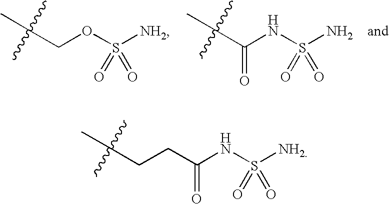

In a preferred embodiment according to the present invention, W and M are nitrogen;
R^{a}, R^{b} and R^{a} are —H;
R³ is —H or C₁-C₆alkyl;
Z is optionally substituted —C₁-C₈ alkyl-;
L is selected from the group consisting of
—C₀-C₆ alkyl-C(O)—N(R³)—C₀-C₃ alkyl-, —C₀-C₆alkyl-N(R³)—C(O)—N(R³)—C₀-C₃alkyl-, or —C₀-C₆alkyl-O—C₀-C₃alkyl-C(O)—N(R³)—C₀-C₃alkyl-, wherein when a C₀-C₃alkyl is C₁-C₃alkyl, the C₁-C₃ alkyl is optionally substituted with —C(O)—N(R³)—C₀-C₃ alkyl-Y, aryl, heteroaryl, -heteroaryl-aryl, -aryl-heteroaryl, -aryl-aryl, heteroaryl-heteroaryl, —N(R³)(R^{3a}) or —N(R³)—Y, wherein each heteroaryl or aryl moeity is optionally substituted;
—C₀-C₆ alkyl-heteroalkyl-C₀-C₆ alkyl-C(O)—N(R³)—C₀-C₃ alkyl-, wherein when the C₀-C₃alkyl is C₁-C₃alkyl, the C₁-C₃ alkyl is optionally substituted with heteroaryl, —N(R³)(R^{3a}) or —N(R³)—Y; and
—C₀-C₆ alkyl-N(R³)C(O)—C₀-C₃ alkyl-, wherein when the C₀-C₃alkyl is C₁-C₃alkyl, the C₁-C₃ alkyl is optionally substituted with —N(R³)(R^{3a}), —N(R³)—Y, —N(R³)—C(O)—O—C₀-C₃alkyl-Y, —NH₂, —NH—S(O)₂—Y, —NH—C(O)—NH—C₀-C₃alkyl-Y, —NH-heteroaryl-aryl or —N(R³)C(O)—C₀-C₃ alkyl-Y; and
Y is selected from the group consisting of H, cycloalkyl, aryl, heterocyclyl, heteroaryl-aryl, aryl-heteroaryl, heterocyclyl-O—, aryl-N(R³)—C(O)-heteroaryl, heteroaryl-N(R³)—C(O)-heteroaryl, aryl-N(R³)—C(O)-aryl, heterocyclyl-C₀-C₆alkyl-N(R³)—C(O)-heteroaryl and B²—B¹—N(R³)—C(O)—C₁-C₇ alkyl-, wherein the alkyl, cycloalkyl, aryl, heteroaryl and heterocyclyl moieties are optionally substituted and the C₁-C₇ alkyl is optionally substituted with —NR³—B³ and the amine of B³ is connected with the acid of B² to form a peptide bond, and wherein when Y is B²—B¹—N(R³)—C(O)—C₁-C₇ alkyl-, then Z and L are covalent bonds; wherein
when any of B¹, B² and B³ are attached together, they are attached by a peptide bond, and B¹, B² and B³ are independently selected from the group consisting of D-Pro, L-ile and D-Phe-4-CF₃.

In a preferred embodiment according to the present invention,
W and M are nitrogen;
R^{a}, R^{b} and R^{a} are —H;
R³ is —H or C₁-C₆alkyl;
Z is optionally substituted —C₁-C₈ alkyl-;
L is selected from the group consisting of
—C₀-C₆ alkyl-C(O)—N(R³)—C₀-C₃ alkyl-, —C₀-C₆alkyl-N(R³)—C(O)—N(R³)—C₀-C₃alkyl-, or —C₀-C₆alkyl-O—C₀-C₃alkyl-C(O)—N(R³)—C₀-C₃alkyl-, wherein when a C₀-C₃alkyl is C₁-C₃alkyl, the C₁-C₃ alkyl is optionally substituted with —C(O)—N(R³)—C₀-C₃ alkyl-Y, -heteroaryl-aryl, heteroaryl, heteroaryl-heteroaryl, —N(R³)(R^{3a}) or —N(R³)—Y, wherein each heteroaryl or aryl moeity is optionally substituted;
—C₀-C₆ alkyl-heteroalkyl-C₀-C₆ alkyl-C(O)—N(R³)—C₀-C₃ alkyl-, wherein when the C₀-C₃alkyl is C₁-C₃alkyl, the C₁-C₃ alkyl is optionally substituted with heteroaryl, —N(R³)(R^{3a}) or —N(R³)—Y; and
—C₀-C₆ alkyl-N(R³)C(O)—C₀-C₃ alkyl-, wherein when the C₀-C₃alkyl is C₁-C₃alkyl, the C₁-C₃ alkyl is optionally substituted with —N(R³)—C(O)—O—C₀-C₃alkyl-Y, —NH₂, —NH—S(O)₂—Y, —NH—C(O)—NH—C₀-C₃alkyl-Y, —NH-heteroaryl-aryl, —N(R³)C(O)—C₀-C₃ alkyl-Y, —N(R³)(R^{3a}) or —N(R³)—Y; and
Y is selected from the group consisting of H, cycloalkyl, aryl, heteroaryl, heteroaryl-aryl, aryl-heteroaryl, heterocyclyl-O—, aryl-N(R³)—C(O)-heteroaryl, heteroaryl-N(R³)—C(O)-heteroaryl, aryl-N(R³)—C(O)-aryl, heterocyclyl-C₀-C₆alkyl-N(R³)—C(O)-heteroaryl and B²—B¹—N(R³)—C(O)—C₁-C₇ alkyl-, wherein the cycloalkyl, aryl, heteroaryl, heterocyclyl and alkyl moieties are optionally substituted with one, two or three (preferably one or two, more preferably one) substituents selected from the group consisting of halo, alkoxy, optionally substituted C₁-C₆alkyl, alkoxycarbonyl-, —OH, —CN, —C(O)—OH, optionally substituted aryl, optionally substituted -alkylaryl, optionally substituted heteroaryl, optionally substituted —O—C₁-C₆alkyl-aryl, optionally substituted —C(O)—O—C₁-C₆alkyl, —NH₂, optionally substituted -aryl-heterocyclyl and optionally substituted fused heterocyle, and the C₁-C₇ alkyl is optionally substituted with —NR³—B³ and the amine of B³ is connected with the acid of B² to form a peptide bond, and wherein when Y is B²—B¹—N(R³)—C(O)—C₁-C₇ alkyl-, then Z and L are covalent bonds; wherein
when any of B¹, B² and B³ are attached together, they are attached by a peptide bond, and B¹, B² and B³ are independently selected from the group consisting of D-Pro, L-ile and D-Phe-4-CF₃.

In a preferred embodiment according to the present invention,
W and M are nitrogen;
R^{a}, R^{b} and R^{a} are —H;
R³ is —H or C₁-C₆alkyl;
Z is optionally substituted —C₁-C₈ alkyl-;
L is
—C₀-C₆ alkyl-N(R³)C(O)—C₀-C₃ alkyl-, wherein when the C₀-C₃alkyl is C₁-C₃alkyl, the C₁-C₃ alkyl is optionally substituted with —N(R³)—C(O)—O—C₀-C₃alkyl-Y, —NH₂, —NH—S(O)₂—Y, —NH—C(O)—NH—C₀-C₃alkyl-Y, —NH-heteroaryl-aryl, —N(R³)C(O)—C₀-C₃ alkyl-Y, —N(R³)(R^{3a}) or —N(R³)—Y; and
Y is selected from the group consisting of H, cycloalkyl, aryl, heteroaryl, heteroaryl-aryl, aryl-heteroaryl, heterocyclyl-O—, aryl-N(R³)—C(O)-heteroaryl, heteroaryl-N(R³)—C(O)-heteroaryl, aryl-N(R³)—C(O)-aryl, heterocyclyl-C₀-C₆alkyl-N(R³)—C(O)-heteroaryl and B²—B¹—N(R³)—C(O)—C₁-C₇ alkyl-, wherein the cycloalkyl, aryl, heteroaryl, heterocyclyl and alkyl groups are optionally substituted and the C₁-C₇ alkyl is optionally substituted with —NR³—B³ and the amine of B³ is connected with the acid of B² to form a peptide bond, and wherein when Y is B²—B¹—N(R³)—C(O)—C₁-C₇ alkyl-, then Z and L are covalent bonds; wherein
when any of B¹, B² and B³ are attached together, they are attached by a peptide bond, and B¹, B² and B³ are independently selected from the group consisting of D-Pro, L-ile and D-Phe-4-CF₃.

In a preferred embodiment according to the present invention,
W and M are nitrogen;
R^{a}, R^{b} and R^{a} are —H;
R³ is —H or C₁-C₆alkyl;
Z is optionally substituted —C₁-C₈ alkyl-;
L is
—C₀-C₆ alkyl-N(R³)C(O)—C₀-C₃ alkyl-, wherein when the C₀-C₃alkyl is C₁-C₃alkyl, the C₁-C₃ alkyl is optionally substituted with —N(R³)—C(O)—O—C₀-C₃alkyl-Y, —NH₂, —NH—S(O)₂—Y, —NH—C(O)—NH—C₀-C₃alkyl-Y, —NH-heteroaryl-aryl, —N(R³)C(O)—C₀-C₃ alkyl-Y.—N(R³)(R^{3a}) or —N(R³)—Y; and
Y is selected from the group consisting of H, cycloalkyl, aryl, heteroaryl, heteroaryl-aryl, aryl-heteroaryl, heterocyclyl-O—, aryl-N(R³)—C(O)-heteroaryl, heteroaryl-N(R³)—C(O)-heteroaryl, aryl-N(R³)—C(O)— aryl, heterocyclyl-C₀-C₆alkyl-N(R³)— C(O)-heteroaryl and B²—B¹—N(R³)—C(O)—C₁-C₇ alkyl-, wherein the cycloalkyl, aryl, heteroaryl, heterocyclyl and alkyl groups are optionally substituted with one, two or three (preferably one or two, more preferably one) substituents selected from the group consisting of halo, alkoxy, optionally substituted C₁-C₆alkyl, alkoxycarbonyl-, —OH, —CN, —C(O)—OH, optionally substituted aryl, optionally substituted -alkylaryl, optionally substituted heteroaryl, optionally substituted —O—C₁-C₆alkyl-aryl, optionally substituted —C(O)—O—C₁-C₆alkyl, —NH₂, optionally substituted -aryl-heterocyclyl and optionally substituted fused heterocyle, and the C₁-C₇ alkyl is optionally substituted with —NR³—B³ and the amine of B³ is connected with the acid of B² to form a peptide bond, and wherein when Y is B²—B¹—N(R³)—C(O)—C₁-C₇ alkyl-, then Z and L are covalent bonds; wherein
when any of B¹, B² and B³ are attached together, they are attached by a peptide bond, and B¹, B² and B³ are independently selected from the group consisting of D-Pro, L-ile and D-Phe-4-CF₃.

In a preferred embodiment according to the present invention,
W and M are nitrogen;
R^{a}, R^{b} and R^{c} are —H;
R³ is —H;
R⁴ is H or F;
Z is optionally substituted —C₁-C₈ alkyl-;
L is selected from the group consisting of
—C₀-C₆ alkyl-C(O)—N(R³)—C₀-C₃ alkyl-, wherein when the C₀-C₃alkyl is C₁-C₃alkyl, the C₁-C₃ alkyl is optionally substituted with —C(O)—N(R³)—C₀-C₃ alkyl-Y, -heteroaryl-aryl, heteroaryl, —N(R³)(R^{3a}) or —N(R³)—Y;
—C₀-C₆ alkyl-heteroalkyl-C₀-C₆ alkyl-C(O)—N(R³)—C₀-C₃ alkyl-, wherein when the C₀-C₃alkyl is C₁-C₃alkyl, the C₁-C₃ alkyl is optionally substituted with heteroaryl, —N(R³)(R^{3a}) or —N(R³)—Y; and
—C₀-C₆ alkyl-N(R³)C(O)—C₀-C₃ alkyl-, wherein when the C₀-C₃alkyl is C₁-C₃alkyl, the C₁-C₃ alkyl is optionally substituted with —N(R³)C(O)—C₀-C₃ alkyl-Y, —N(R³)(R^{3a}) or —N(R³)—Y; and
Y is selected from the group consisting of aryl, heteroaryl, heteroaryl-aryl, aryl-heteroaryl and B²—B¹—N(R³)—C(O)—C₁-C₇ alkyl-, wherein the aryl and heteroaryl are optionally substituted, and the C₁-C₇ alkyl is optionally substituted with —NR³—B³ and the amine of B³ is connected with the acid of B² to form a peptide bond, and wherein when Y is B 2-B¹—N(R³)—C(O)—C₁-C₇ alkyl-, then Z and L are covalent bonds; wherein
when any of B¹, B² and B³ are attached together, they are attached by a peptide bond, and
B¹, B² and B³ are independently selected from the group consisting of D-Pro, L-ile and D-Phe-4-CF₃.

In a preferred embodiment according to the present invention,
W and M are nitrogen;
R^{a}, R^{b} and R^{c} are —H;
R³ is —H;
R⁴ is H or F;
Z is optionally substituted —C₁-C₈ alkyl-;
L is selected from the group consisting of
—C₀-C₆ alkyl-C(O)—N(R³)—C₀-C₃ alkyl-, wherein when the C₀-C₃alkyl is C₁-C₃alkyl, the C₁-C₃ alkyl is optionally substituted with —C(O)—N(R³)—C₀-C₃ alkyl-Y, -heteroaryl-aryl, heteroaryl, —N(R³)(R^{3a}) or —N(R³)—Y;
—C₀-C₆ alkyl-heteroalkyl-C₀-C₆ alkyl-C(O)—N(R³)—C₀-C₃ alkyl-, wherein when the C₀-C₃alkyl is C₁-C₃alkyl, the C₁-C₃ alkyl is optionally substituted with heteroaryl, —N(R³)(R^{3a}) or —N(R³)—Y; and
—C₀-C₆ alkyl-N(R³)C(O)—C₀-C₃ alkyl-, wherein when the C₀-C₃alkyl is C₁-C₃alkyl, the C₁-C₃ alkyl is optionally substituted with —N(R³)C(O)—C₀-C₃ alkyl-Y, —N(R³)(R^{3a}) or —N(R³)—Y; and
Y is selected from the group consisting of aryl, heteroaryl, heteroaryl-aryl, aryl-heteroaryl and B²—B¹—N(R³)—C(O)—C₁-C₇ alkyl-, wherein the aryl and heteroaryl groups are optionally substituted with one, two or three (preferably one or two, more preferably one) substituents selected from the group consisting of halo, alkoxy, optionally substituted C₁-C₆alkyl, alkoxycarbonyl-, —OH, —CN, —C(O)—OH, optionally substituted aryl, optionally substituted -alkylaryl, optionally substituted heteroaryl, optionally substituted —O—C₁-C₆alkyl-aryl, optionally substituted —C(O)—O—C₁-C₆alkyl, —NH₂, optionally substituted -aryl-heterocyclyl and optionally substituted fused heterocyle, and the C₁-C₇ alkyl is optionally substituted with —NR³—B³ and the amine of B³ is connected with the acid of B² to form a peptide bond, and wherein when Y is B²—B¹—N(R³)—C(O)—C₁-C₇ alkyl-, then Z and L are covalent bonds; wherein
when any of B¹, B² and B³ are attached together, they are attached by a peptide bond, and B¹, B² and B³ are independently selected from the group consisting of D-Pro, L-ile and D-Phe-4-CF₃.

In a preferred embodiment according to the present invention,
W and M are nitrogen;
R^{a}, R^{b} and R^{a} are —H;
R³ is —H;
R⁴ is H or F;
Z is optionally substituted —C₁-C₈ alkyl-;
L is
—C₀-C₆ alkyl-N(R³)C(O)—C₀-C₃ alkyl-, wherein when the C₀-C₃alkyl is C₁-C₃alkyl, the C₁-C₃ alkyl is optionally substituted with —N(R³)C(O)—C₀-C₃ alkyl-Y, —N(R³)(R^{3a}) or —N(R³)—Y; and
Y is selected from the group consisting of aryl, heteroaryl, heteroaryl-aryl, aryl-heteroaryl and B²—B¹—N(R³)—C(O)—C₁-C₇ alkyl-, wherein the aryl and heteroaryl groups are optionally substituted, and the C₁-C₇ alkyl is optionally substituted with —NR³—B³ and the amine of B³ is connected with the acid of B² to form a peptide bond, and wherein when Y is B²—B¹—N(R³)—C(O)—C₁-C₇ alkyl-, then Z and L are covalent bonds; wherein
when any of B¹, B² and B³ are attached together, they are attached by a peptide bond, and
B¹, B² and B³ are independently selected from the group consisting of D-Pro, L-ile and D-Phe-4-CF₃.

In a preferred embodiment according to the present invention,
W and M are nitrogen;
R^{a}, R^{b} and R^{a} are —H;
R³ is —H;
R⁴ is H or F;
Z is optionally substituted —C₁-C₈ alkyl-;
L is
—C₀-C₆ alkyl-N(R³)C(O)—C₀-C₃ alkyl-, wherein when the C₀-C₃alkyl is C₁-C₃alkyl, the C₁-C₃ alkyl is optionally substituted with —N(R³)C(O)—C₀-C₃ alkyl-Y, —N(R³)(R^{3a}) or —N(R³)—Y; and
Y is selected from the group consisting of aryl, heteroaryl, heteroaryl-aryl, aryl-heteroaryl and B²—B¹—N(R³)—C(O)—C₁-C₇ alkyl-, wherein the aryl and heteroaryl groups are optionally substituted with one, two or three (preferably one or two, more preferably one) substituents selected from the group consisting of halo, alkoxy, optionally substituted C₁-C₆alkyl, alkoxycarbonyl-, —OH, —CN, —C(O)—OH, optionally substituted aryl, optionally substituted -alkylaryl, optionally substituted heteroaryl, optionally substituted —O—C₁-C₆alkyl-aryl, optionally substituted —C(O)—O—C₁-C₆alkyl, —NH₂, optionally substituted -aryl-heterocyclyl and optionally substituted fused heterocyle, and the C₁-C₇ alkyl is optionally substituted with —NR³—B³ and the amine of B³ is connected with the acid of B² to form a peptide bond, and wherein when Y is B²—B¹—N(R³)—C(O)—C₁-C₇ alkyl-, then Z and L are covalent bonds; wherein
   when any of B¹, B² and B³ are attached together, they are attached by a peptide bond, and B¹, B² and B³ are independently selected from the group consisting of D-Pro, L-ile and D-Phe-4-CF₃.

In a preferred embodiment according to the present invention, a substituent selected from the group consisting of optionally substituted aryl, optionally substituted -alkylaryl, optionally substituted heteroaryl, optionally substituted —O—C₁-C₆alkyl-aryl, optionally substituted —C(O)—O—C₁-C₆alkyl, optionally substituted -aryl-heterocyclyl and optionally substituted fused heterocyle is itself further optionally substituted on an alkyl, aryl, heteroaryl or heterocylclyl moiety with a substituent selected from the group consisting of —O—C₁-C₆alkyl-alkoxy, —CF₃, —O-aryl, alkoxy, —NH—C(O)—C₁-C₆alkyl, halogen, C₁-C₆alkyl, —O-(halo substituted alkyl) and —O-alkyl-N(alkyl)₂.

In a preferred embodiment according to the present invention, a substituent selected from the group consisting of optionally substituted aryl and optionally substituted heteroaryl, is itself further optionally substituted with a substituent selected from the group consisting of —O—C₁-C₆alkyl-alkoxy, —CF₃, —O-aryl, alkoxy, —NH—C(O)—C₁-C₆alkyl, halogen, C₁-C₆alkyl, —O-(halo substituted alkyl) and —O-alkyl-N(alkyl)₂.

In a preferred embodiment according to the present invention, Y is further selected from heterocyclyl.

In a preferred embodiment according to the present invention L is —C₀-C₆ alkyl-C(O)—N(R³)—C₀-C₃ alkyl-, wherein when the C₀-C₃alkyl is C₁-C₃alkyl, the C₁-C₃ alkyl is optionally substituted with aryl, heteroaryl, -heteroaryl-aryl, -aryl-heteroaryl, -aryl-aryl or heteroaryl-heteroaryl, wherein each heteroaryl or aryl moeity is optionally substituted; and

Y is aryl or heteroaryl, each of which is optionally substituted.

In a preferred embodiment according to the present invention the compound is selected from the group consisting of

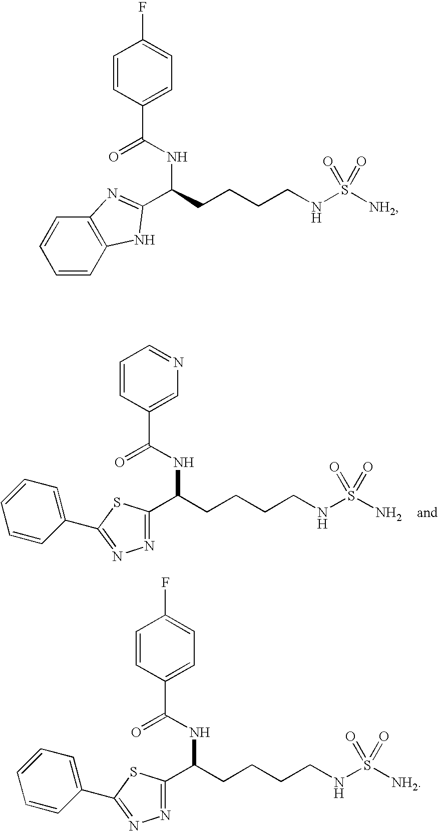

In a preferred embodiment according to the present invention
L is —C₀-C₆alkyl-O—C₀-C₁alkyl-C(O)—N(R³)—C₀-C₃alkyl-, wherein when the C₀-C₃alkyl is C₁-C₃alkyl, the C₁-C₃ alkyl is optionally substituted with -heteroaryl-aryl, -heteroaryl-heteroaryl, heteroaryl, -heteroaryl-heterocylcyl, wherein each heteroaryl and aryl moeity is optionally substituted; and
Y is optionally substituted aryl.

In a preferred embodiment according to the present invention when a C₀-C₃alkyl is C₁-C₃alkyl, the C₁-C₃ alkyl is optionally substituted with -heteroaryl-aryl, -heteroaryl-heteroaryl, heteroaryl, -heteroaryl-heterocylcyl, wherein each heteroaryl and aryl moeity is further optionally substituted with 1 to 3 of optionally substituted aryl, alkoxy, —N(alkyl)₂, halogen, alkyl, fused heterocyclyl, —CF₃, optionally substituted heterocyclyl, —O—C₁-C₆alkyl-N(alkyl)₂, —O—C₁-C₆alkyl-NH₂ and —NH-aryl.

In a preferred embodiment according to the present invention the compound is selected from the group consisting of

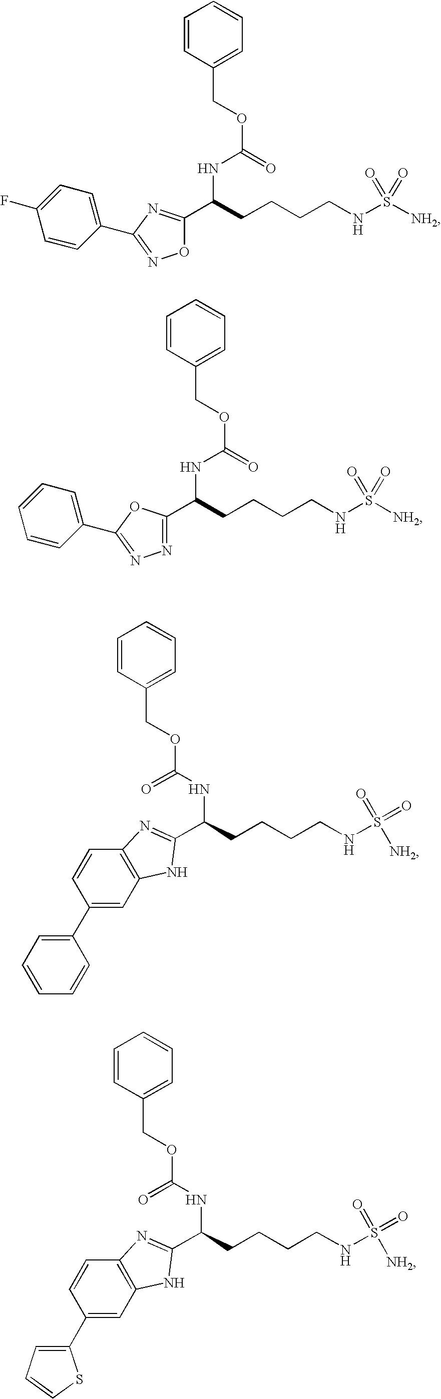

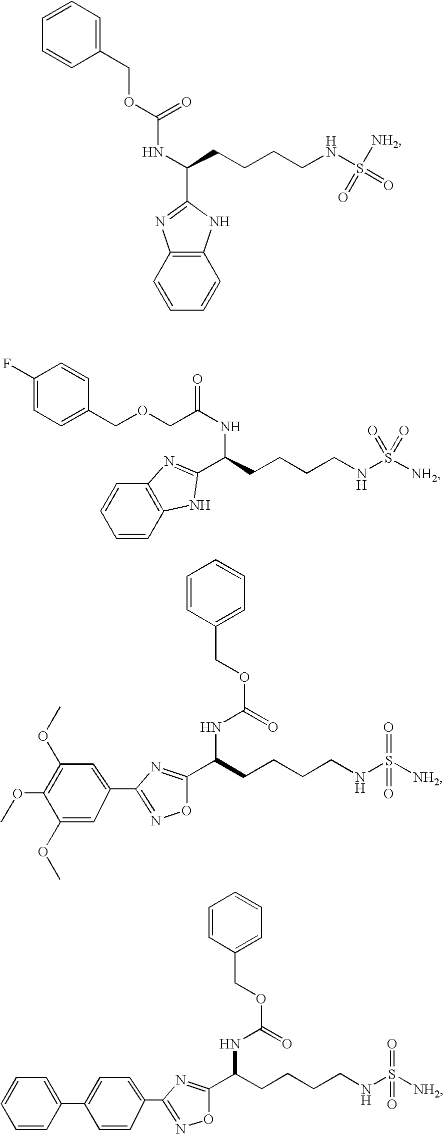

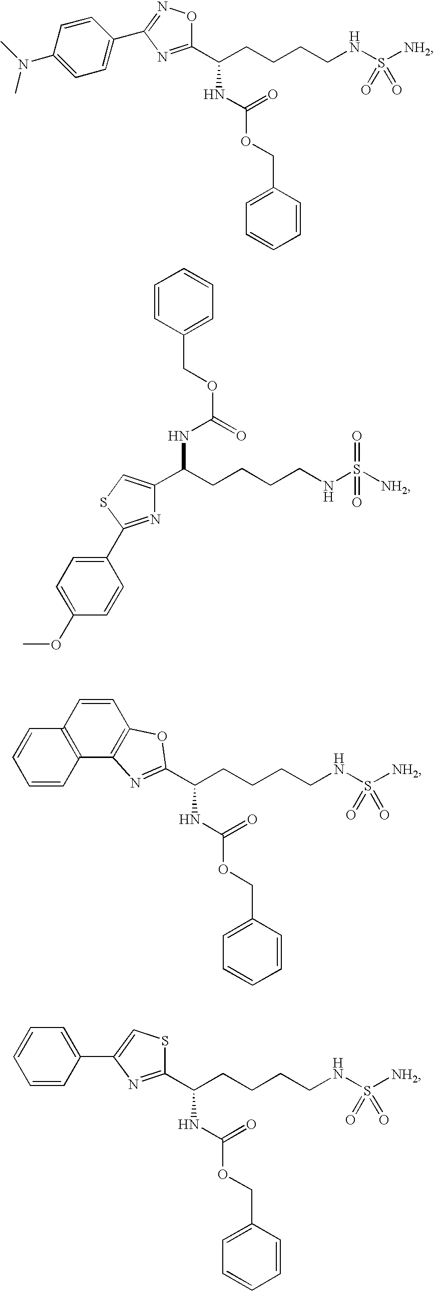

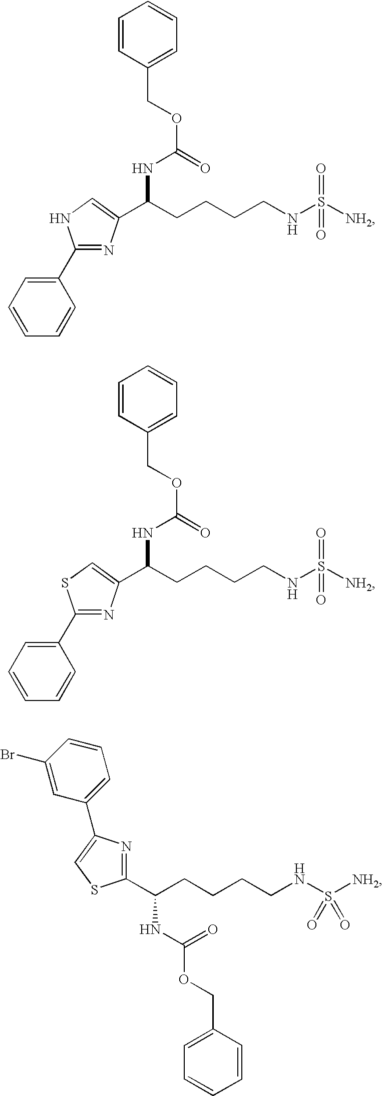

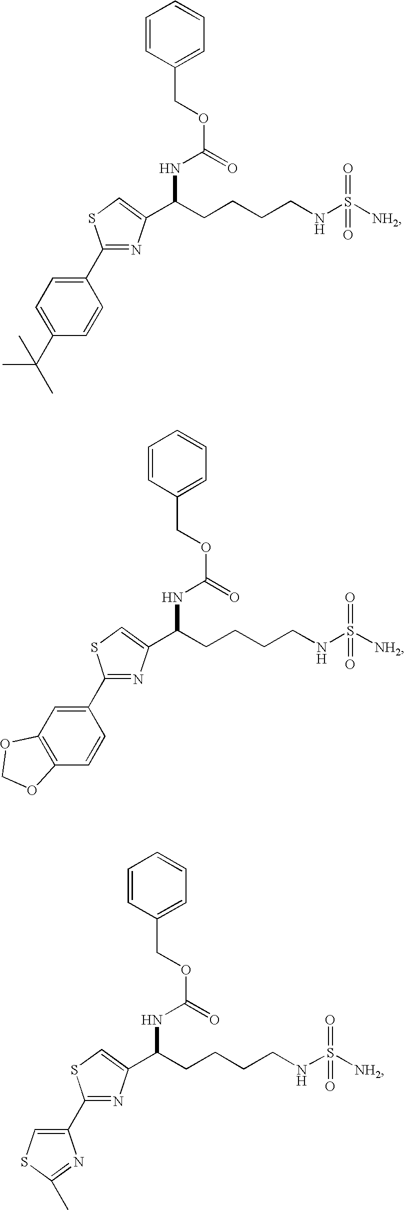

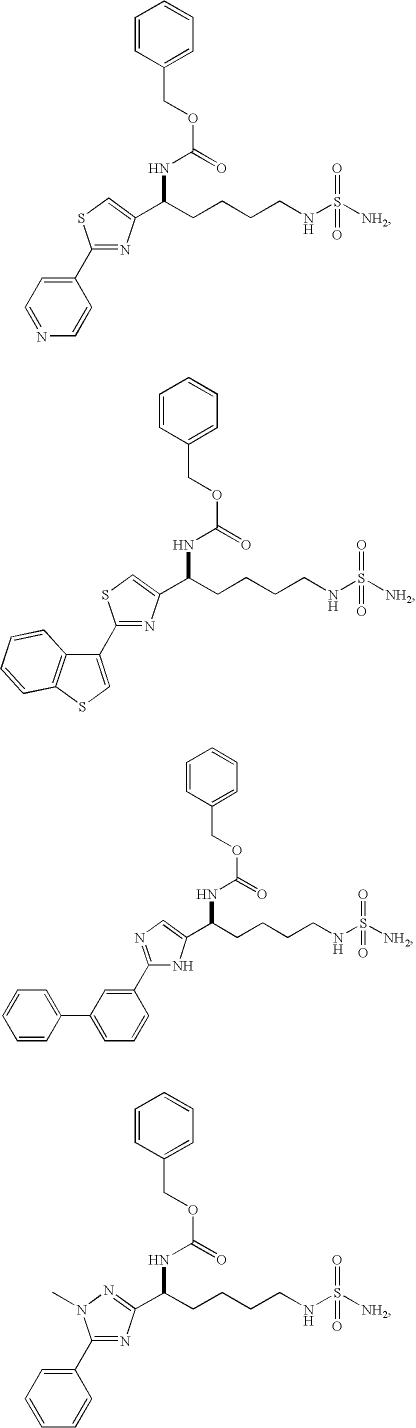

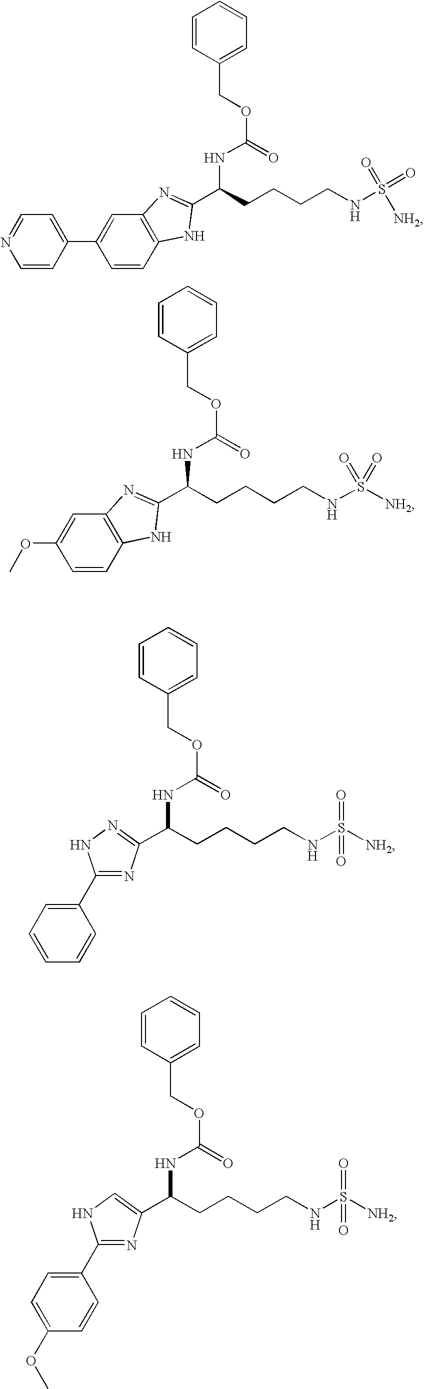

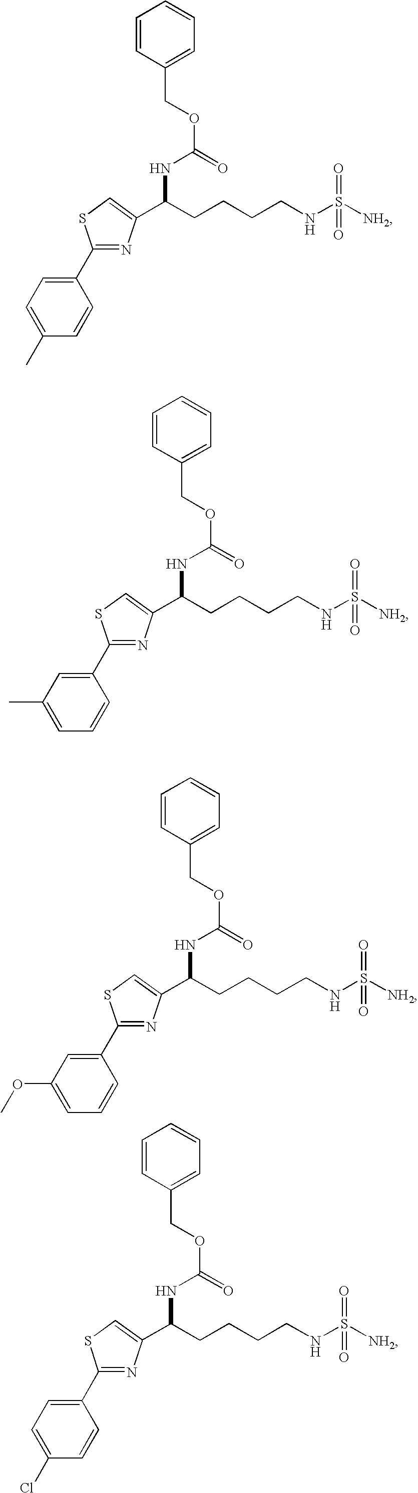

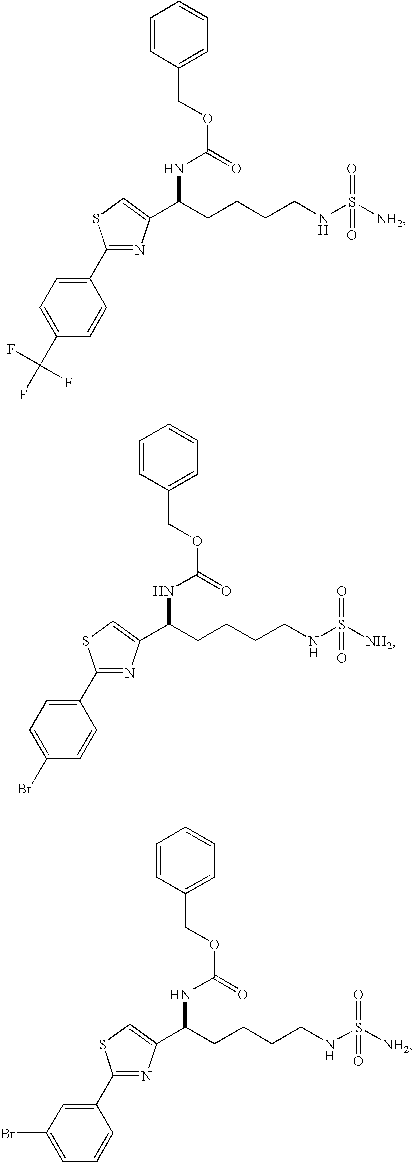

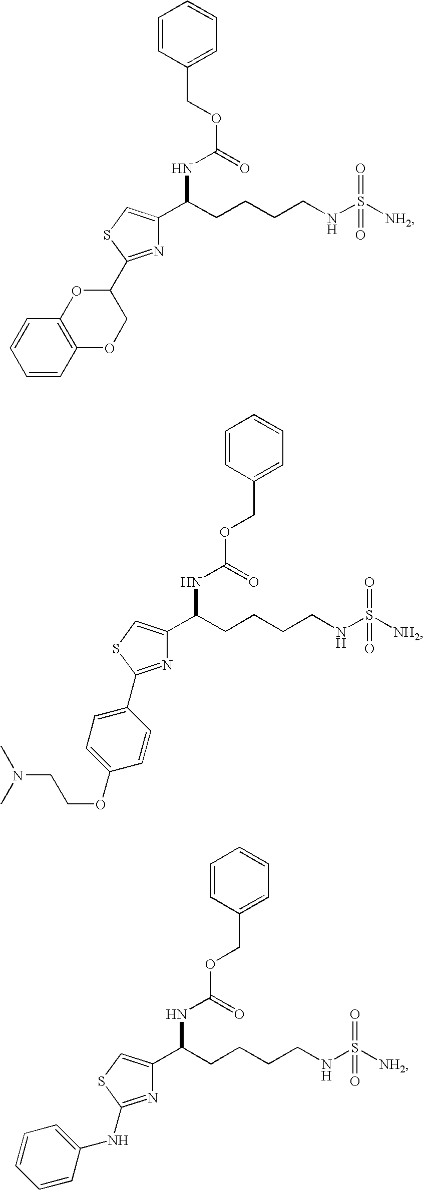

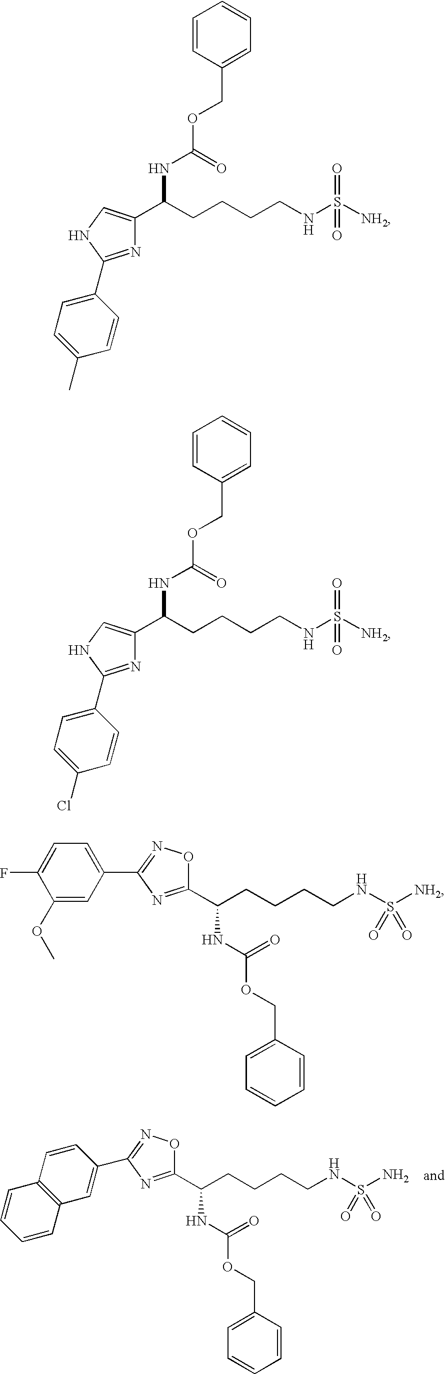

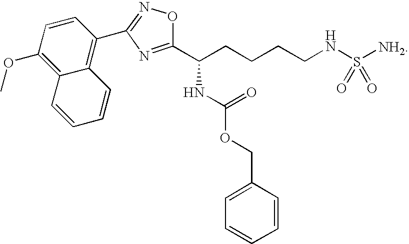

In a preferred embodiment according to the present invention
L is —C₀-C₆ alkyl-C(O)—N(R³)—C₀-C₃ alkyl-, wherein when the C₀-C₃alkyl is C₁-C₃alkyl, the C₁-C₃ alkyl is optionally substituted with —C(O)—N(R³)—C₀-C₃alkyl-heteroaryl, —C(O)—N(R³)—C₀-C₃alkyl-aryl, wherein each heteroaryl or aryl moeity is optionally substituted; and
Y is H, optionally substituted aryl or optionally substituted heterocyclyl.

In a preferred embodiment according to the present invention, Y is optionally substituted heteroaryl.

In a preferred embodiment according to the present invention, Y is optionally substituted aryl or optionally substituted heteroaryl, wherein each heteroaryl or aryl moeity is optionally substituted with 1 or 2 independently selected halogen, alkyl or alkoxy.

In a preferred embodiment according to the present invention, the compound is selected from the group consisting of

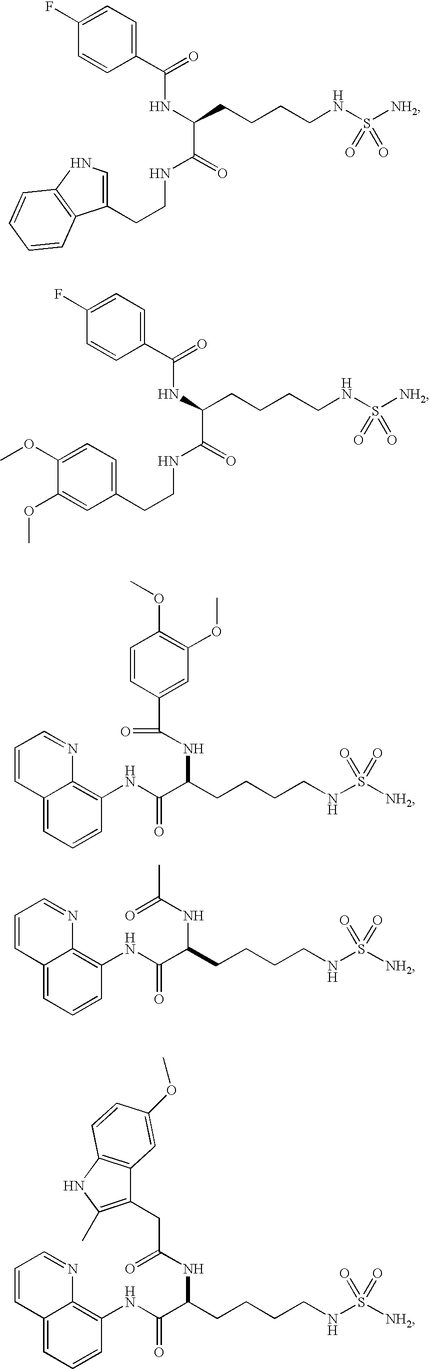

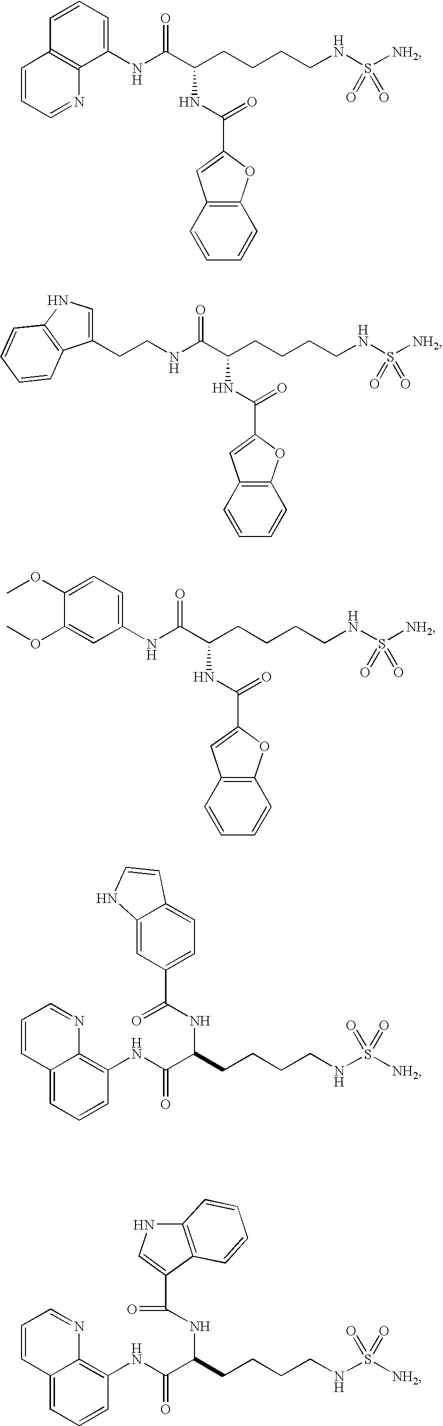

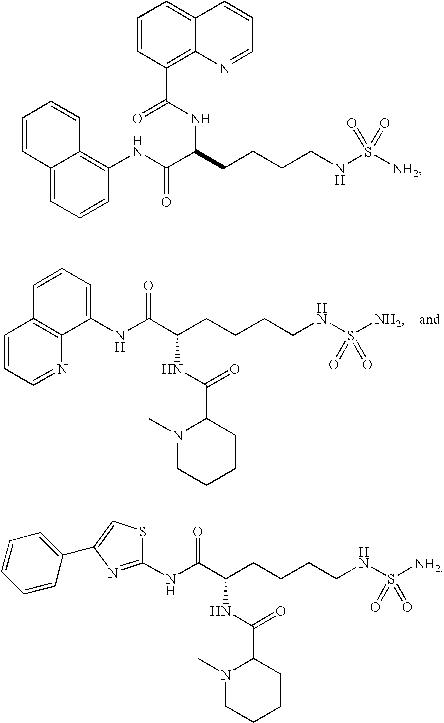

In a preferred embodiment according to the present invention,

L is —C₀-C₆alkyl-O—C₀-C₁alkyl-C(O)—N(R³)—C₀-C₃alkyl-, wherein when the C₀-C₃alkyl is C₁-C₃alkyl, the C₁-C₃ alkyl is optionally substituted with —C(O)—N(R³)—C₀-C₃ alkyl-heterocyclyl or —C(O)—N(R³)—C₀-C₃ alkyl-aryl, wherein each heterocyclyl or aryl moeity is optionally substituted; and

Y is optionally substituted aryl or optionally substituted heteroaryl.

In a preferred embodiment according to the present invention, Y is optionally substituted aryl.

In a preferred embodiment according to the present invention, —C(O)—N(R³)—C₀-C₃ alkyl-heterocyclyl is —C(O)—N(R³)—C₀-C₃ alkyl-heteroaryl.

In a preferred embodiment according to the present invention, Y-L- is phenyl-CH₂—O—C(O)—NH—.

In a preferred embodiment according to the present invention,

L is —C₀-C₆alkyl-O—C₀-C₁alkyl-C(O)—N(R³)—C₀-C₃alkyl-, wherein when the C₀-C₃alkyl is C₁-C₃alkyl, the C₁-C₃ alkyl is optionally substituted with —C(O)—N(R³)—C₀-C₃ alkyl-heteroaryl or —C(O)—N(R³)—C₀-C₃ alkyl-aryl, wherein each heteroaryl or aryl moeity is optionally substituted with 1 to 3 independent substituents selected from the group consisting of halogen, —OH, —NH₂, alkyl, —C(O)—OH, —C(O)—O-alkyl, —C(O)—NH-optionally substituted aryl, —C(O)—NH-optionally substituted heteroaryl, —C(O)—NH-alkyl-O-alkyl, —C(O)—NH-alkyl-heterocyclyl, -alkyl-optionally substituted aryl, alkoxy, optionally substituted aryl, optionally substituted heteroaryl.

In a preferred embodiment according to the present invention, wherein substituents selected from the group consisting of —C(O)—NH-optionally substituted aryl, —C(O)—NH-optionally substituted heteroaryl, -alkyl-optionally substituted aryl, optionally substituted aryl and optionally substituted heteroaryl, are optionally substituted with 1 or 2 independently selected substituents selected from the group consisting of halogen, alkoxy, alkyl, —O-aryl, —NH—C(O)-alkyl, oxo, —CN, heterocyclyl, —O-halosubstitutedalkyl, —CF₃ and —O-alkyl-O-alkyl.

In a preferred embodiment according to the present invention, L is phenyl-CH₂—O—C(O)—NH—C₁-C₃alkyl-, wherein the C₁-C₃ alkyl is substituted with —C(O)—NH-thiazolyl, wherein the thiazolyl is optionally substituted.

In a preferred embodiment according to the present invention,

L is phenyl-CH₂—O—C(O)—NH—C₁-C₃alkyl-, wherein the C₁-C₃ alkyl is substituted with —C(O)—NH-thiazolyl, wherein the thiazolyl is optionally substituted with 1 or 2 independently selected substituents selected from the group consisting of optionally substituted aryl, alkyl, —C(O)—O-alkyl, —C(O)—OH, —C(O)—NH-optionally substituted aryl, —C(O)—NH-optionally substituted heteroaryl, —C(O)—NH-alkyl-O-alkyl, —C(O)—NH-alkyl-heterocyclyl, fused optionally substituted cycloalkyl, fused optionally substituted heterocyclyl and fused optionally substituted aryl.

In a preferred embodiment according to the present invention, the compound is selected from the group consisting of

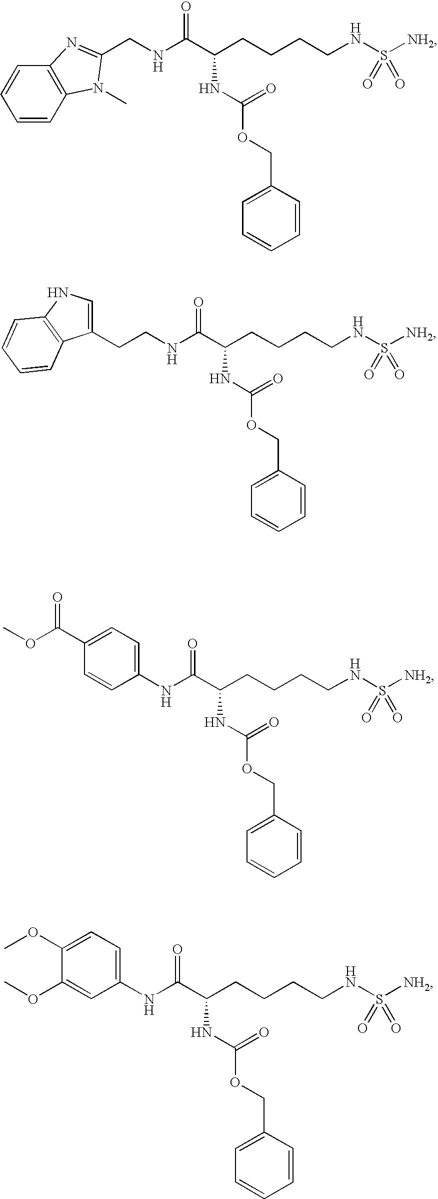

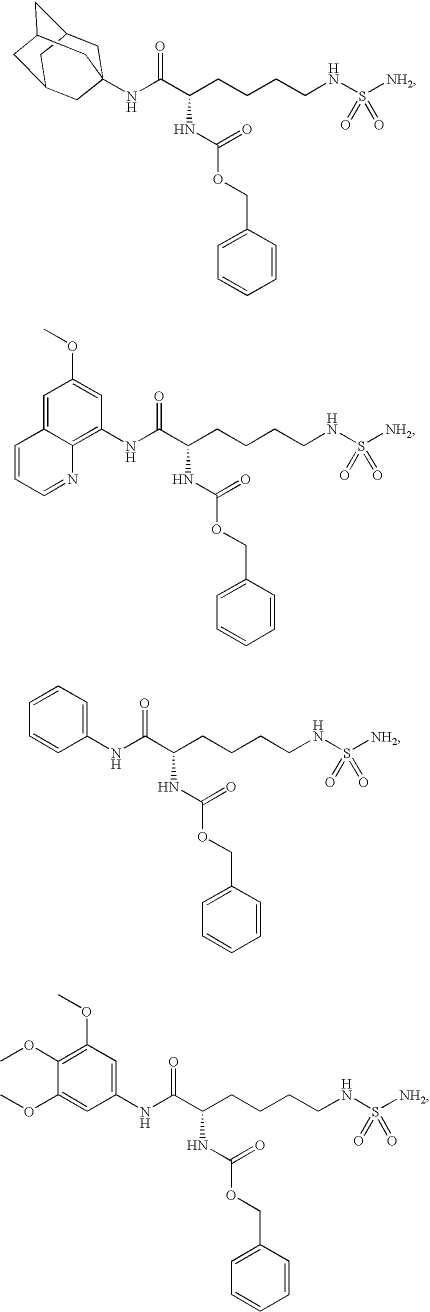

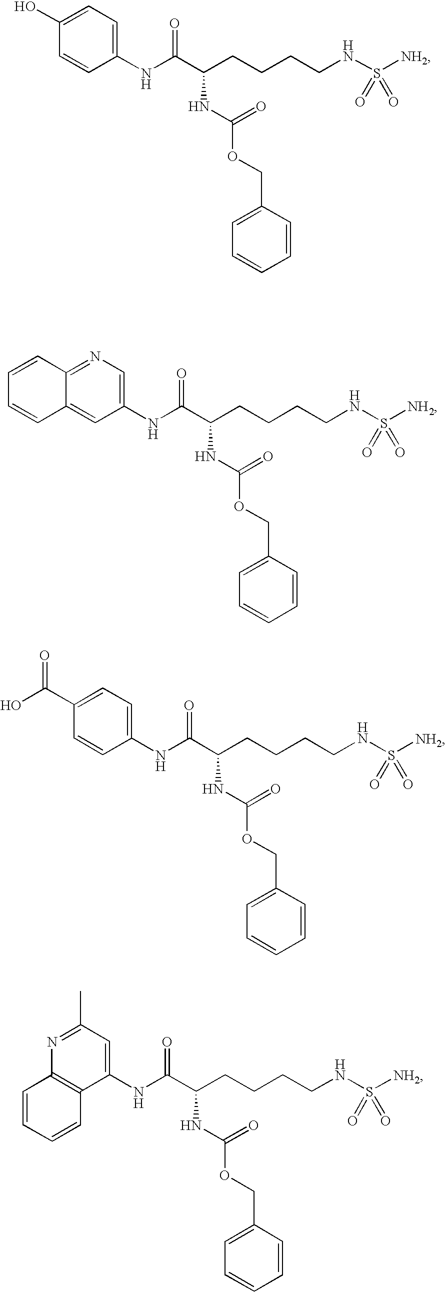

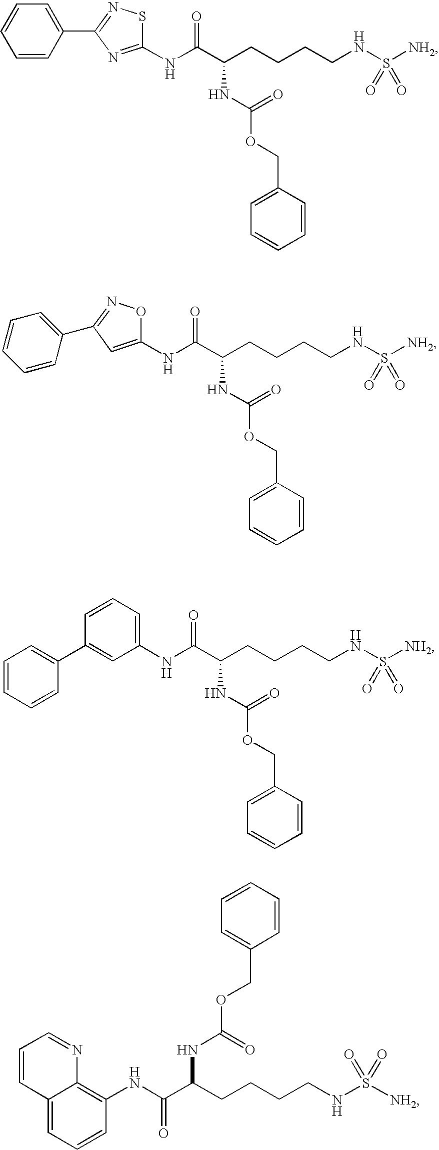

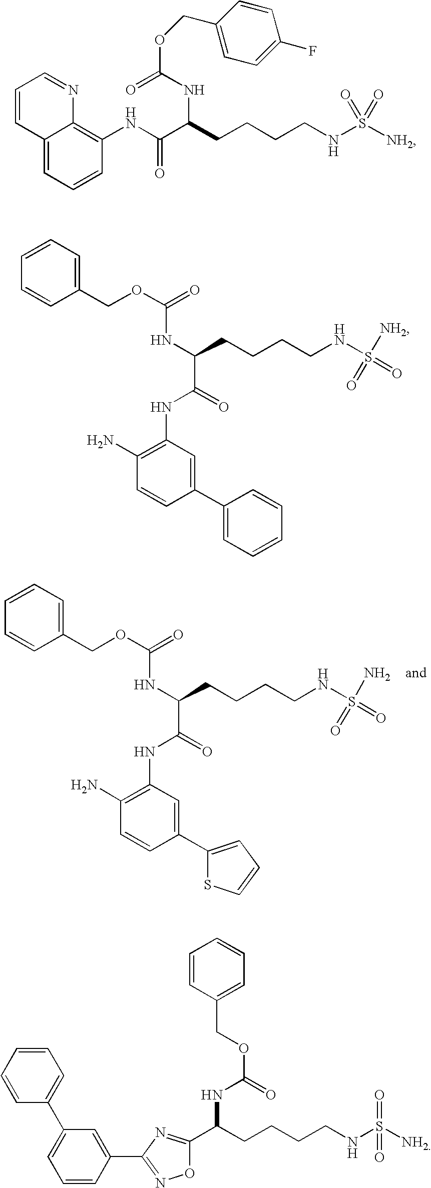

In a preferred embodiment according to the present invention, the compound is selected from the group consisting of

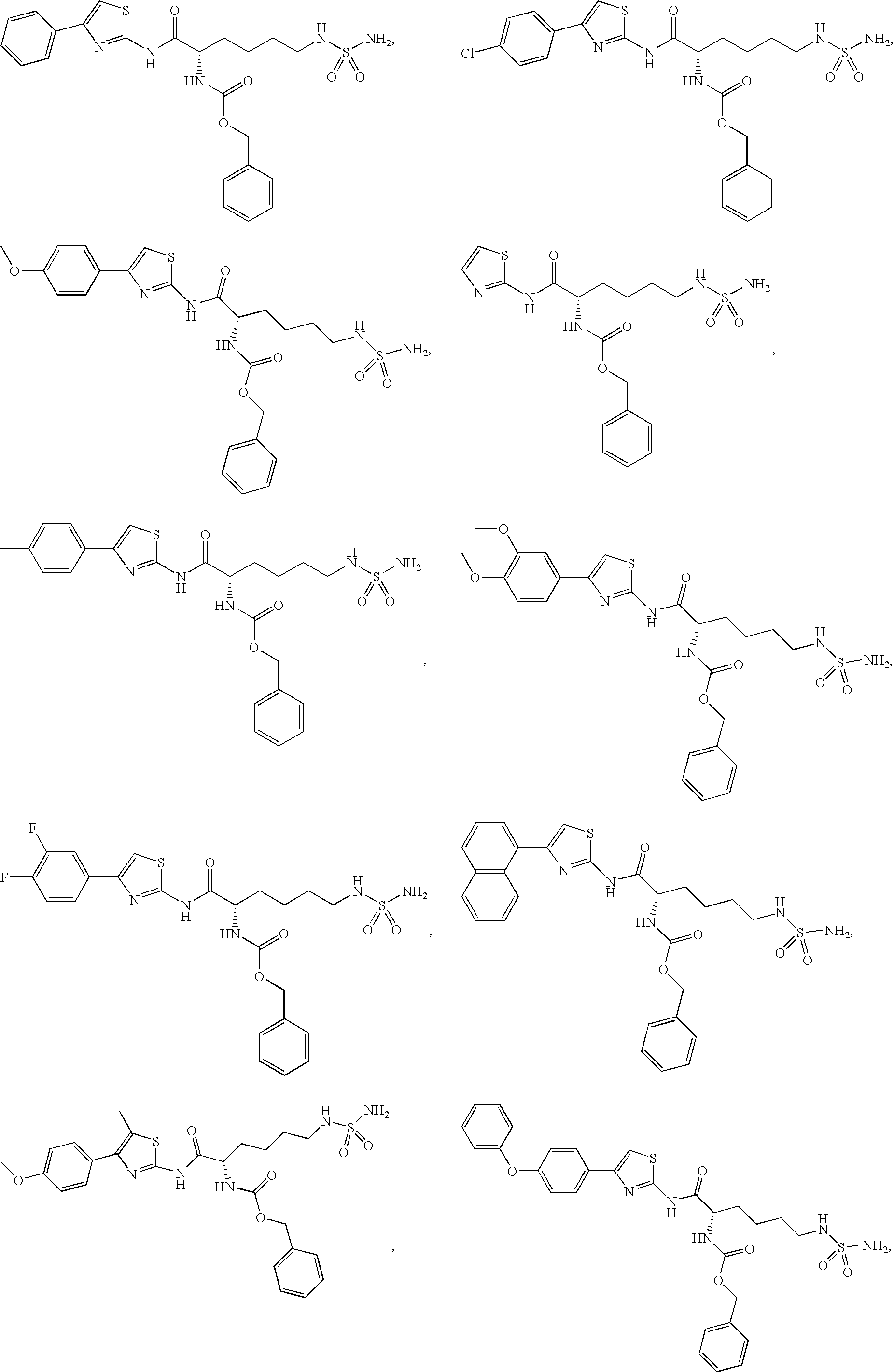

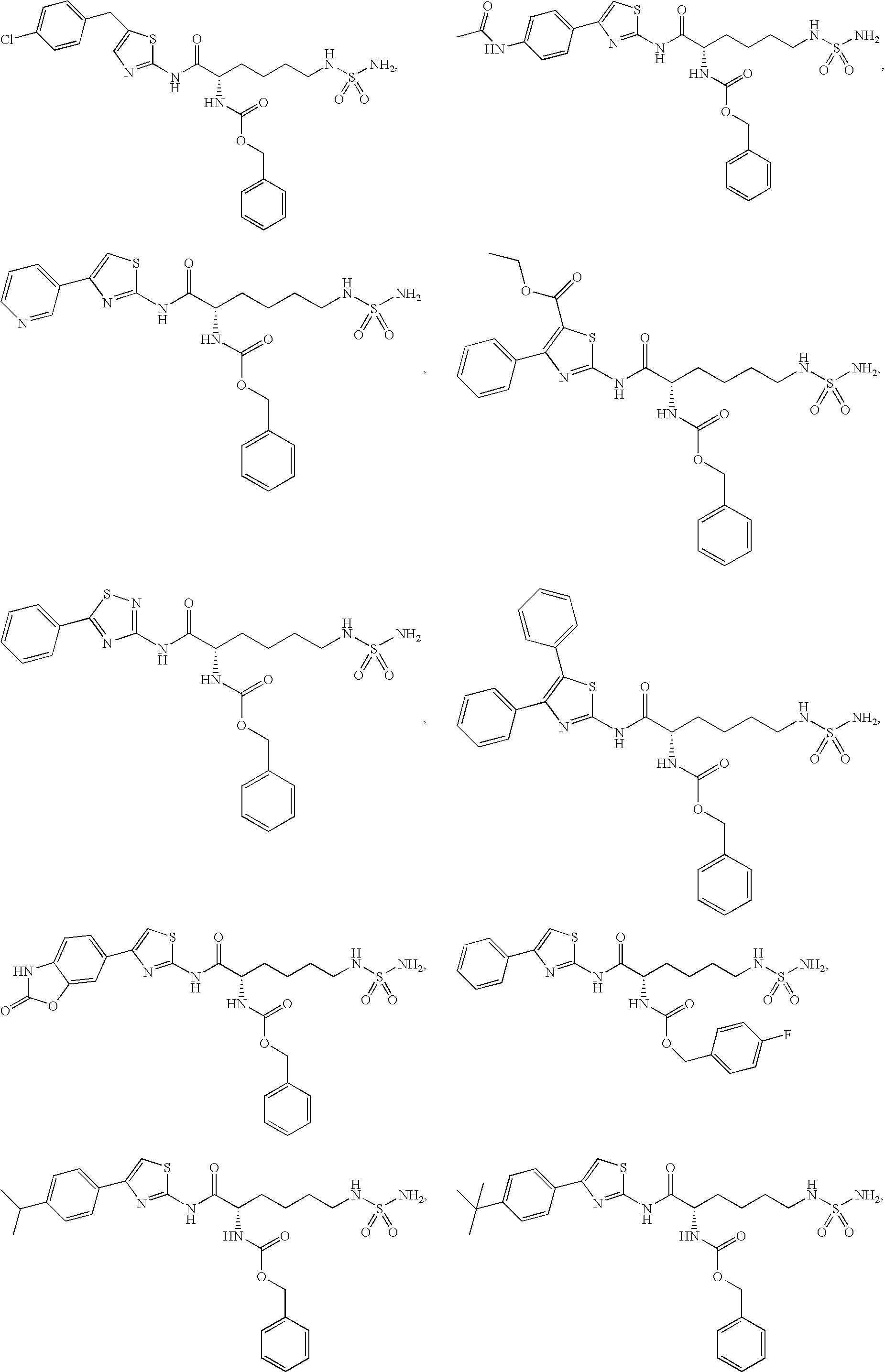

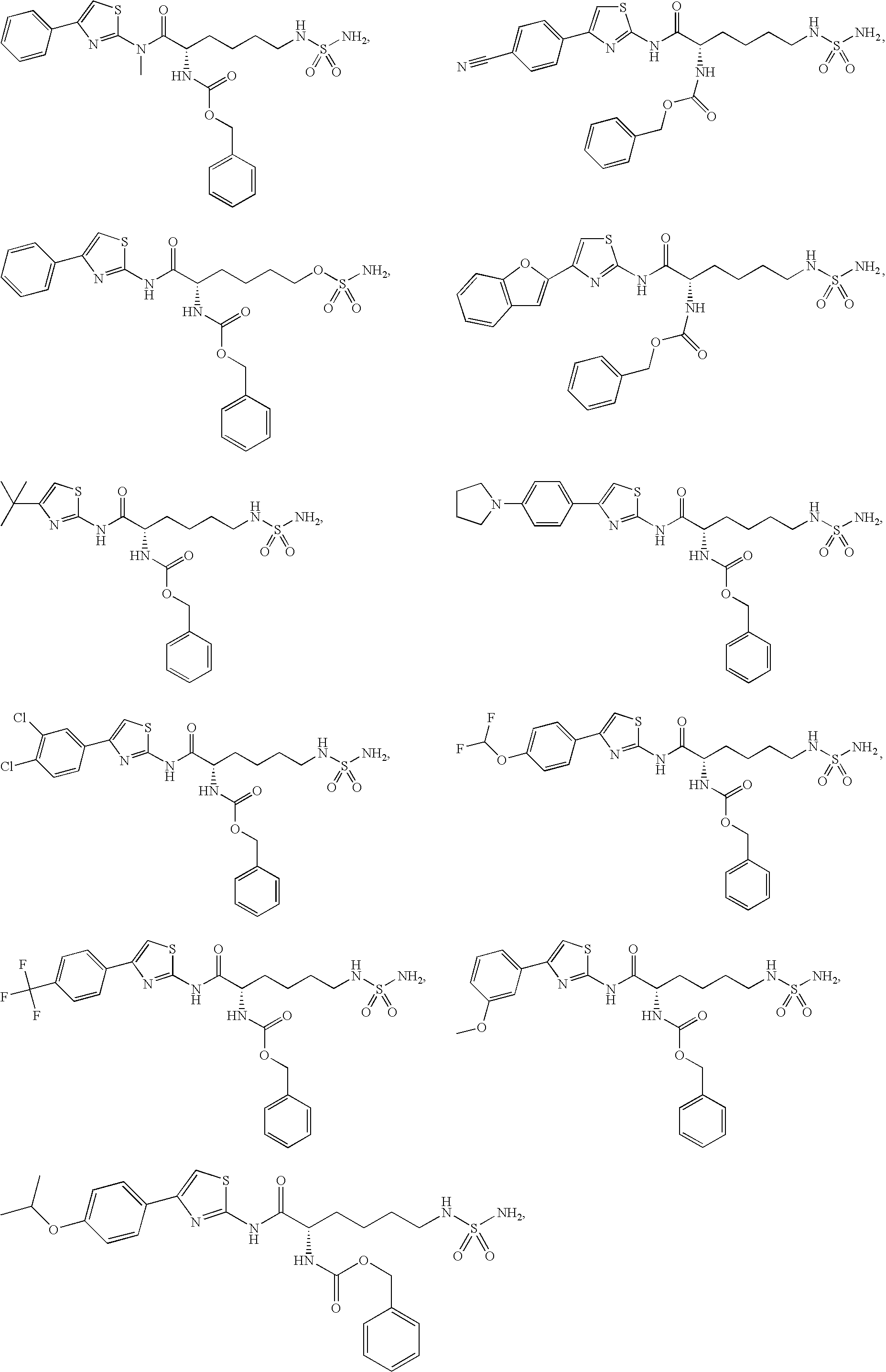

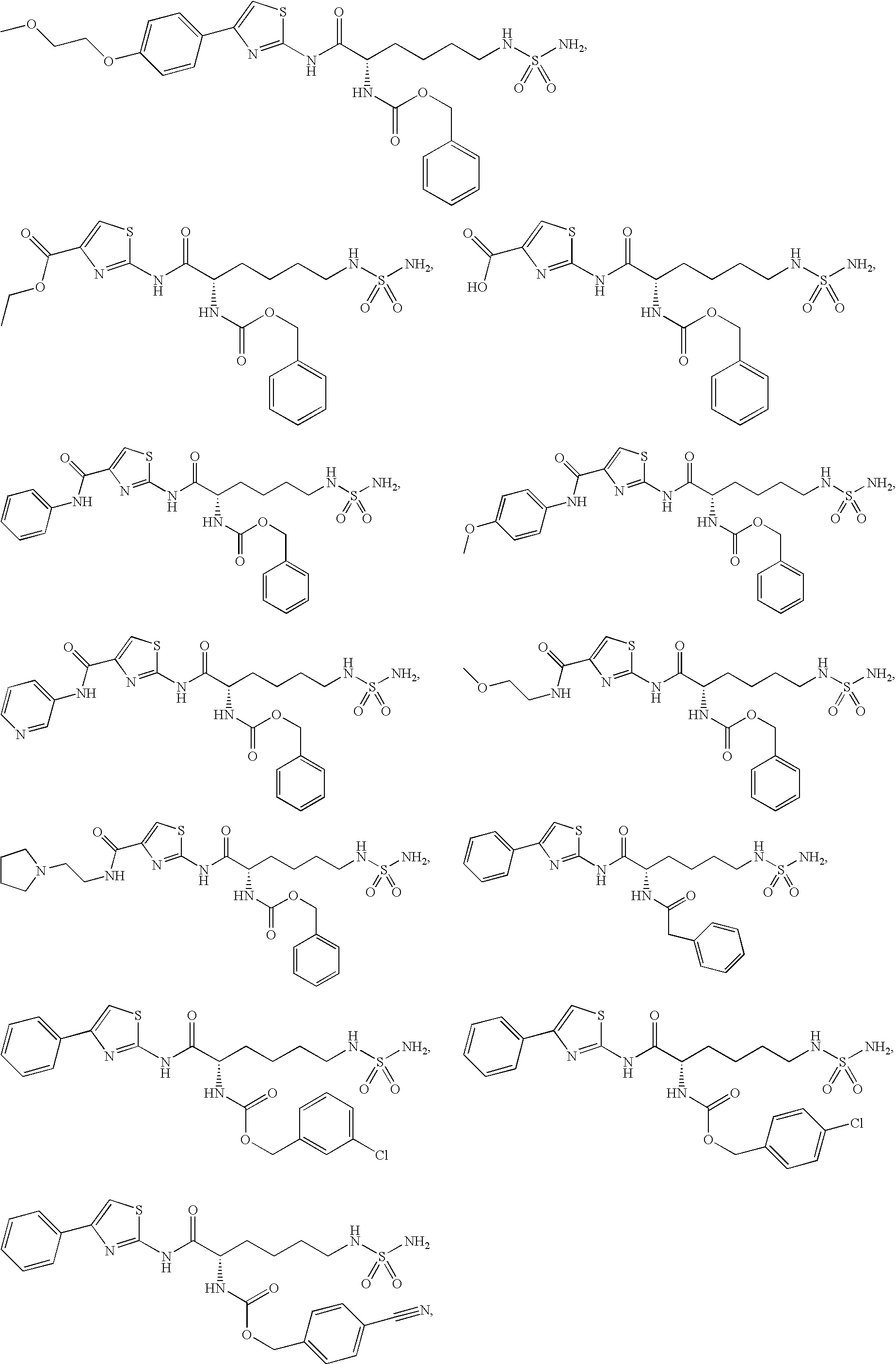

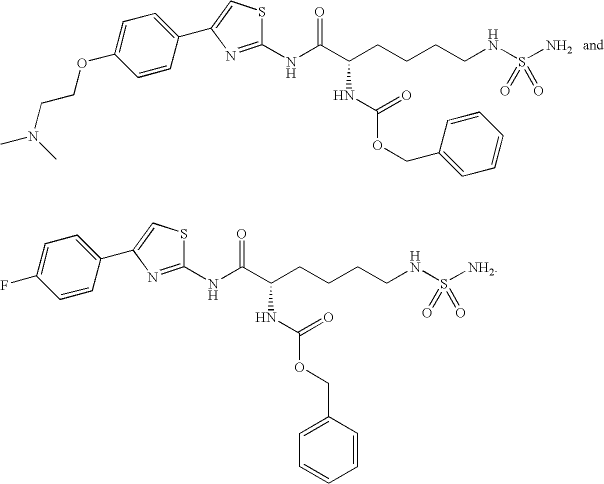

In a preferred embodiment according to the present invention, the compound is selected from the group consisting of

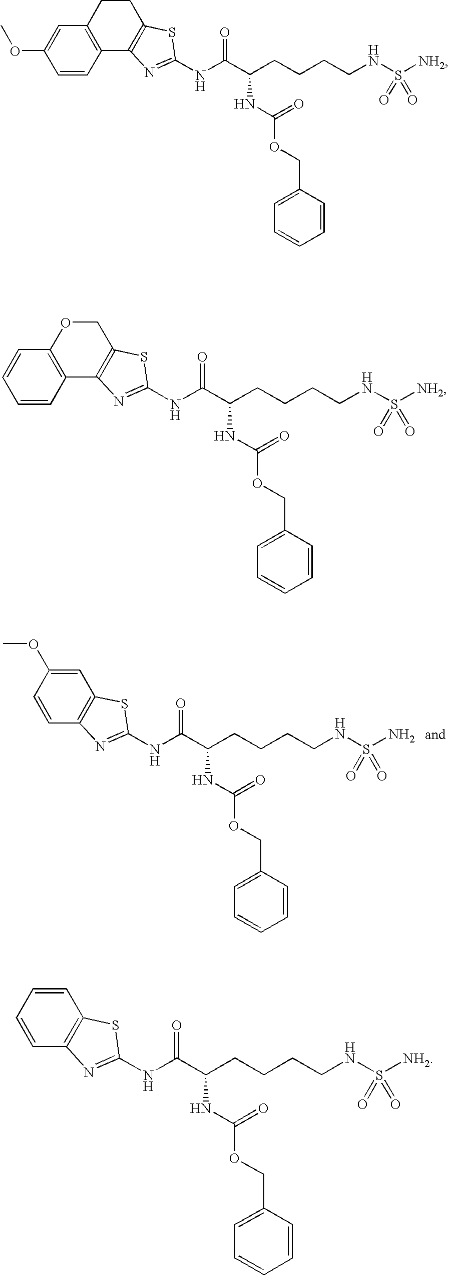

In a preferred embodiment according to the present invention, the compound is selected from the group consisting of

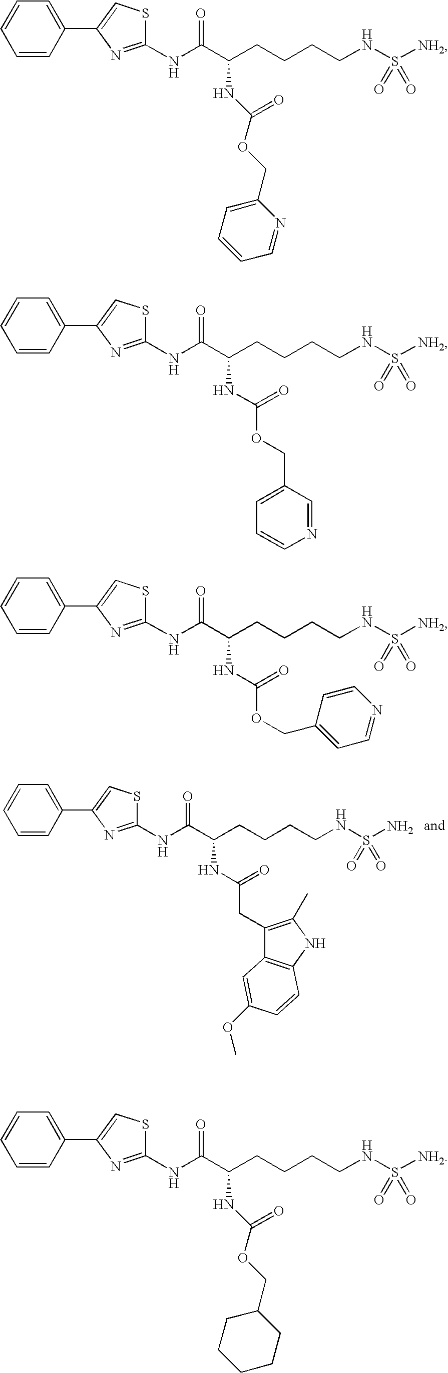

In a preferred embodiment according to the present invention,
L is —C₀-C₆alkyl-N(R³)—C(O)—N(R³)—C₀-C₃alkyl-,wherein when the C₀-C₃alkyl is C₁-C₃alkyl, the C₁-C₃ alkyl is optionally substituted with —C(O)—N(R³)—C₀-C₃alkyl-heteroaryl-aryl, —C(O)—N(R³)—C₀-C₃alkyl-heteroaryl, or —C(O)—N(R³)—C₀-C₃alkyl-aryl, wherein each heteroaryl or aryl moeity is optionally substituted; and
Y is optionally substituted aryl, optionally substituted heterocyclyl or optionally substituted cycloalkyl.

In a preferred embodiment according to the present invention, Y is an optionally substituted heteroaryl.

In a preferred embodiment according to the present invention, the compound is selected from the group consisting of

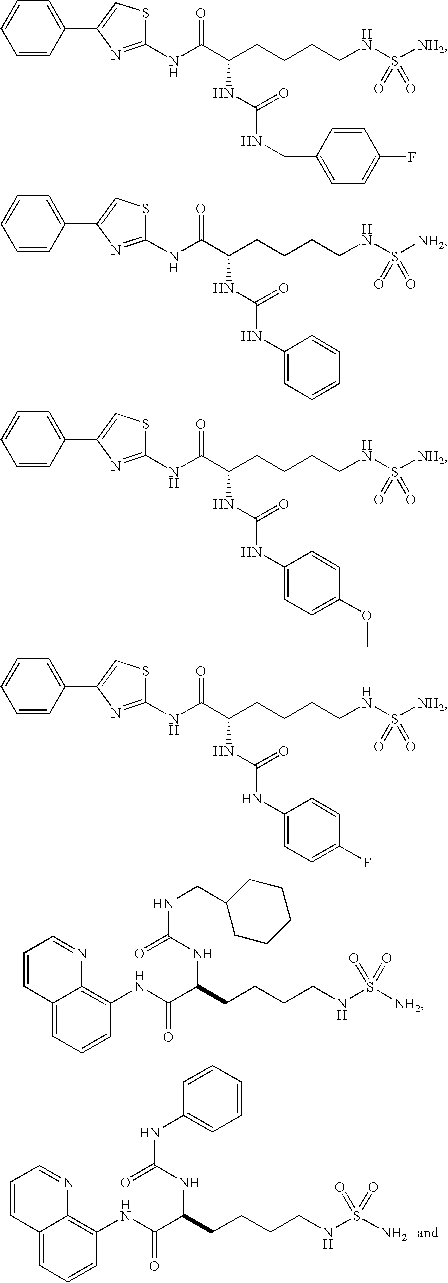

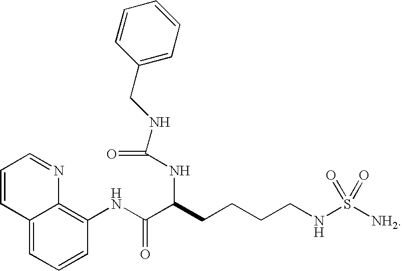

In a preferred embodiment according to the present invention,
L is —C₀-C₆alkyl-O—C₀-C₃alkyl-C(O)—N(R³)—C₀-C₃alkyl-, wherein when a C₀-C₃alkyl is C₁-C₃alkyl, the C₁-C₃ alkyl is optionally substituted with —C(O)—N(R³)—C₀-C₃alkyl-heteroaryl, —C(O)—N(R³)—C₀-C₃alkyl-aryl, —C(O)—N(R³)—C₀-C₃alkyl-heteroaryl-aryl, —C(O)—N(R³)—C₀-C₃alkyl-heteroaryl-heteroaryl, —C(O)—N(R³)—C₀-C₃alkyl-aryl-aryl and —C(O)—N(R³)—C₀-C₃alkyl-aryl-heteroaryl, wherein each heteroaryl or aryl moeity is optionally substituted; and
Y is H.

In a preferred embodiment according to the present invention, the compound is selected from the group consisting of

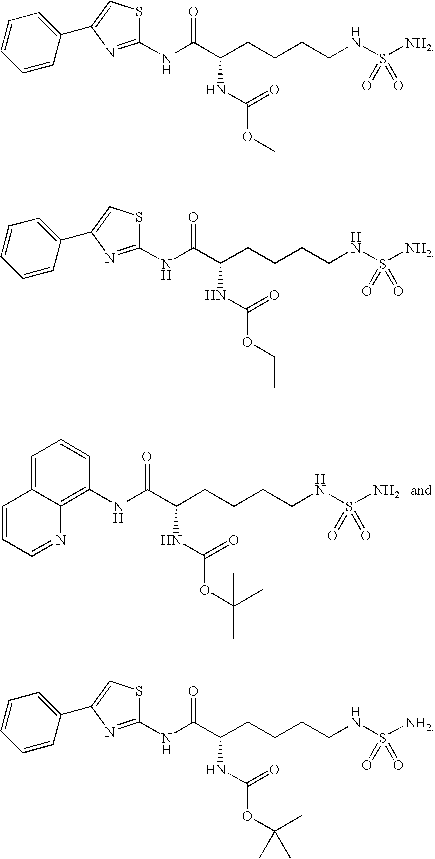

In a preferred embodiment according to the present invention, W is further selected from O.

In a preferred embodiment according to the present invention, the compounds are represented by the formula (IV):

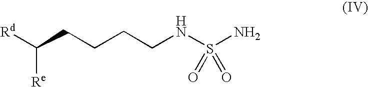

or an N-oxide, hydrate, solvate, pharmaceutically acceptable salt, prodrug or complex thereof, wherein R^{d} and R^{e} are any one of the following combinations:

| R^{d} | R^{e} |
|---|---|
| 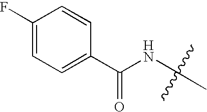 | 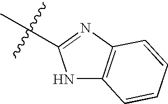 |
| 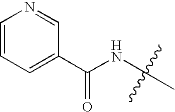 | 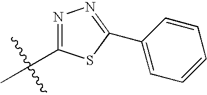 |
| 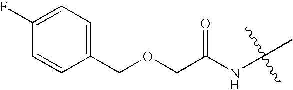 | 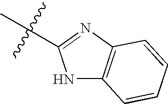 |
| 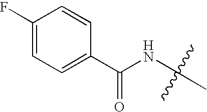 | 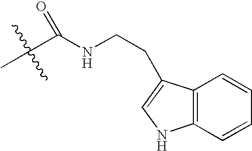 |
| 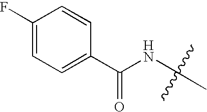 | 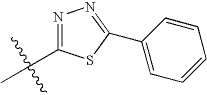 |
| 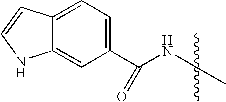 | 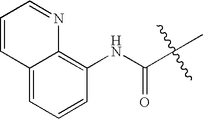 |
| 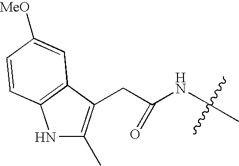 | 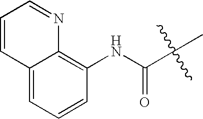 |

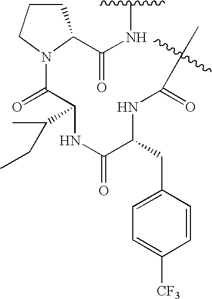

In a preferred embodiment according to the present invention, the compounds are represented by the formula (V) 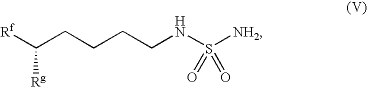
or an N-oxide, hydrate, solvate, pharmaceutically acceptable salt, prodrug or complex thereof wherein

R^{f} and R^{g} are selected from the group consisting of the following combinations:

| R^{g} | R^{f} |
|---|---|
| 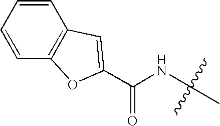 | 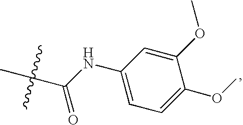 |
| 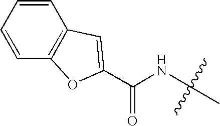 | 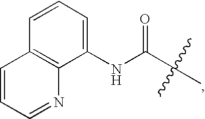 |
| 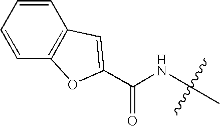 | 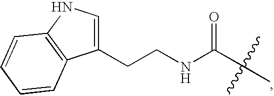 |
| 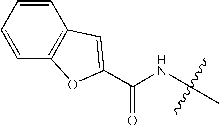 | 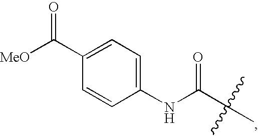 |
| 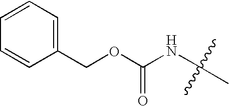 | 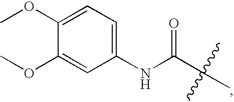 |
| 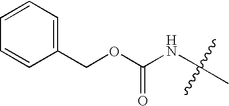 | 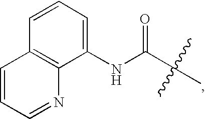 |
| 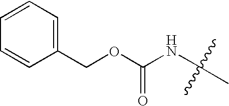 | 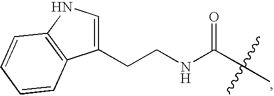 |
| 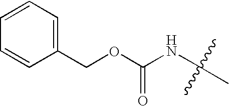 | 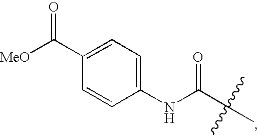 |
| 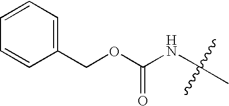 | 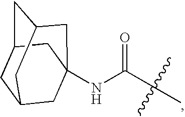 |
| 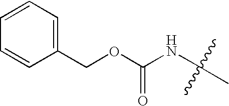 | 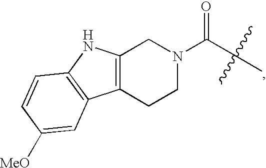 |
| 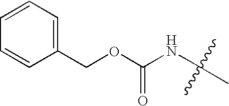 | 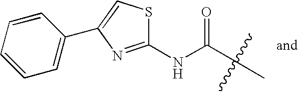 |
| 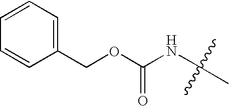 | 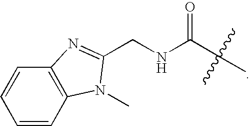 |

In a preferred embodiment according to the present invention, 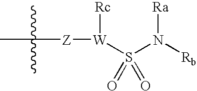
is the structure

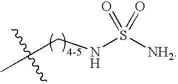

In a preferred embodiment according to the present invention, 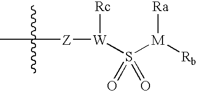
is the structure

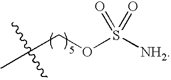

In a preferred embodiment according to the present invention, 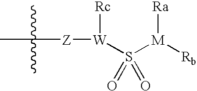
is the structure

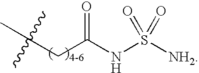

In a preferred embodiment according to the present invention, the compound is selected from the group consisting of

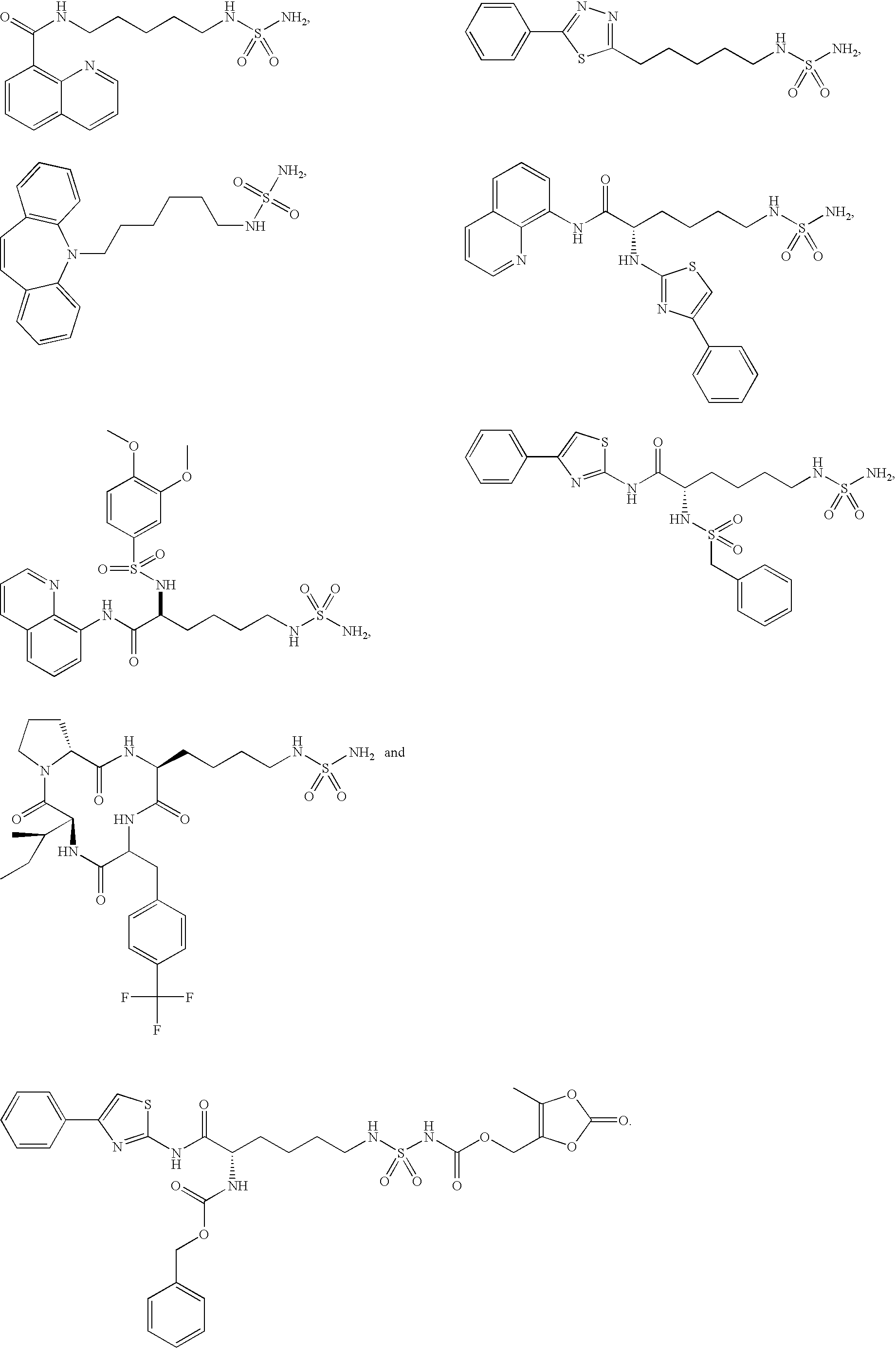

In a preferred embodiment according to the present invention, the compound is selected from the group consisting of

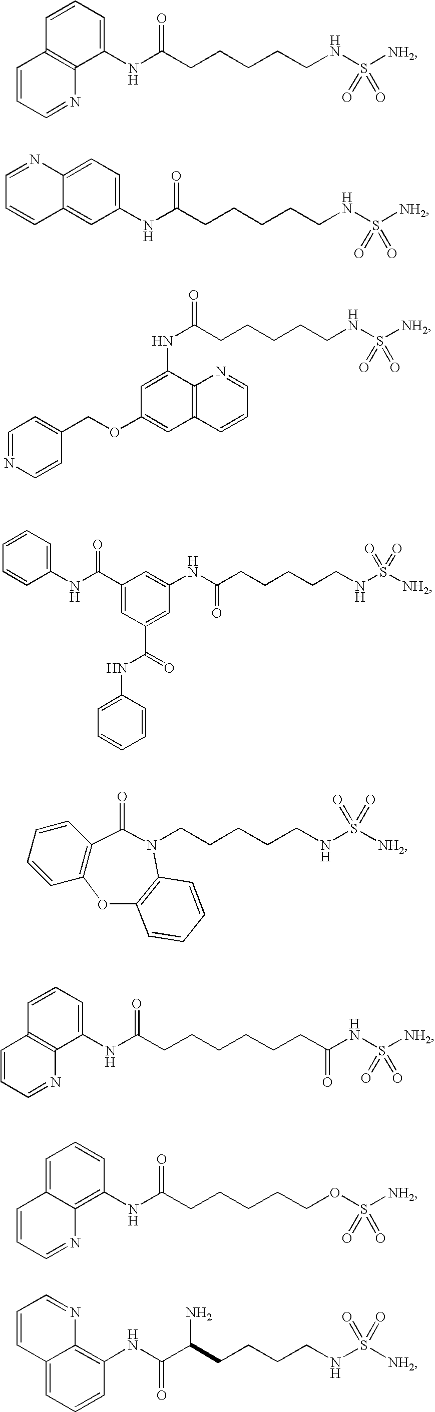

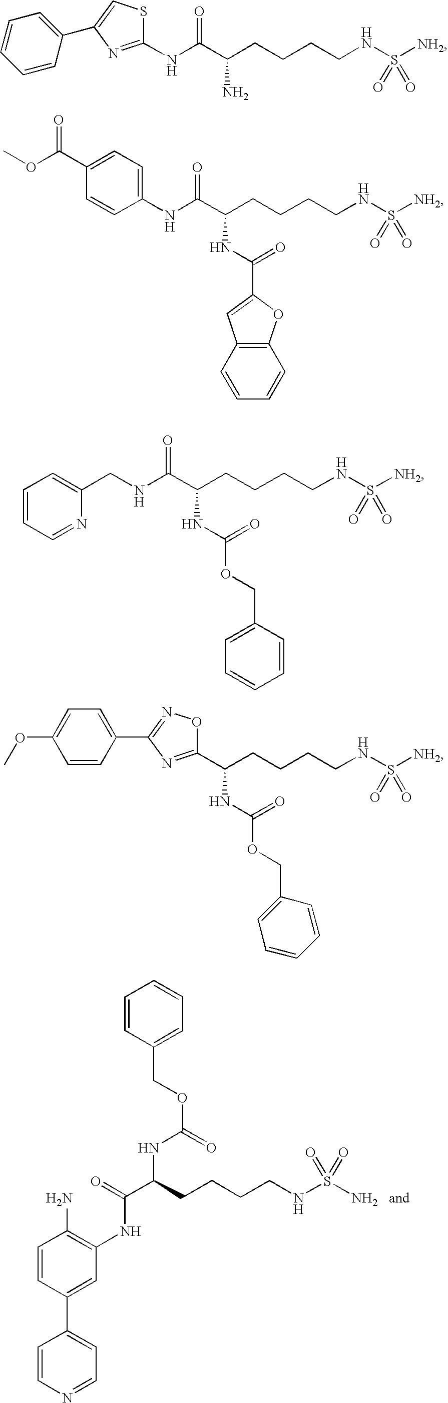

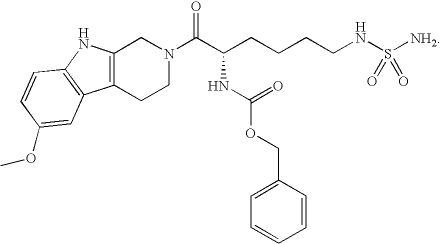

Some examples of the compounds according to the first aspect of the invention are listed in the table below. These examples merely serve to exemplify some of the compounds of the first aspect of the invention and do not limit the scope of the invention.

### SYNTHETIC SCHEMES AND EXPERIMENTAL PROCEDURES

The compounds of the invention can be prepared according to the reaction schemes for the examples illustrated below utilizing methods known to one of ordinary skill in the art. These schemes serve to exemplify some procedures that can be used to make the compounds of the invention. One skilled in the art will recognize that other general synthetic procedures may be used. The compounds of the invention can be prepared from starting components that are commercially available. Any kind of substitutions can be made to the starting components to obtain the compounds of the invention according to procedures that are well known to those skilled in the art.

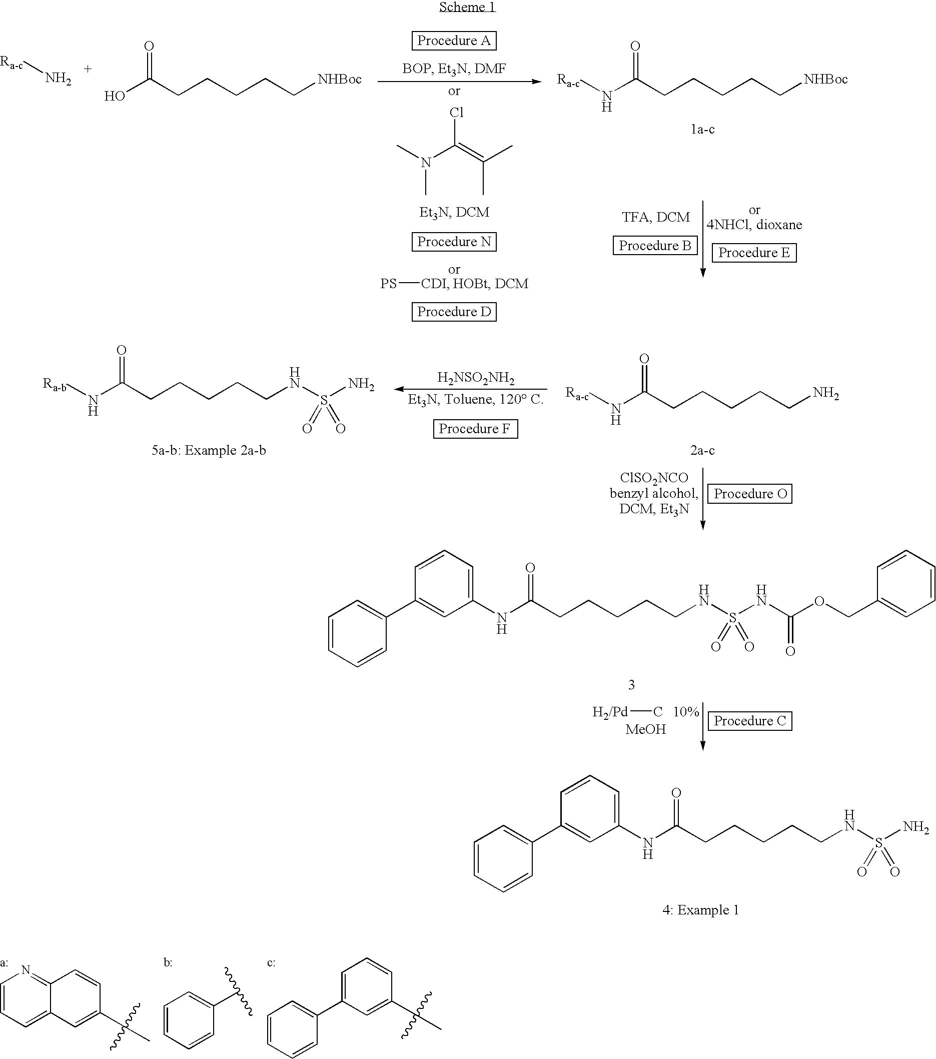

### Example 1

### N-(Biphenyl-3-yl)-6-(sulfamoylamino)hexanamide (4)

### Step 1: tert-Butyl 6-(biphenyl-3-ylamino)-6-oxohexylcarbamate (1c)

N-Boc-caproic acid (1.1 g, 4.77 mmol), 3-phenyl aniline (806 mg, 4.77 mmol) and BOP (2.11 g, 4.77 mmol) were dissolved in DMF (10 ml). Triethylamine (11.92 mmol, 1.66 ml) was added and the reaction was stirred for 3 hours at room temperature. The reaction was then quenched with water and extracted with ethyl acetate. The organic extract was dried (Na₂SO₄), filtered, and evaporated. The residue was purified by silica gel column chromatography with gradient of EtOAc (25-100%) in Hexane to afford 1c (1.65 g, 91%) as a beige solid. LRMS (ESI): (calc.) 382.2; (found) 383.3 (MH)⁺.

### Step 2: 6-Amino-N-(biphenyl-3-yl)hexanamide (2c)

To a solution of 1c (1.23 g, 3.22 mmol) in DCM (40 ml) was added TFA (6 ml). The mixture was stirred for 2 hours. The reaction was basified with NaHCO₃ (ss) and extracted with ethyl acetate. The organic layer was dried (Na₂SO₄), filtered, and evaporated to afford 2c (0.90 g, 98%) as viscous colorless oil. LRMS (ESI): (calc.) 282.5; (found) 283.0 (MH)⁺.

### Step 3: Benzyl N-(6-(biphenyl-3-ylamino)-6-oxohexyl)sulfamoylcarbamate (R³)

To a stirred solution of sulfurisocyanatidic chloride (0.10 ml, 1.16 mmol) in DCM (2 ml) at 0° C. was added benzyl alcohol (0.12 ml, 1.16 mmol). The resulting solution was stirred for 30 minutes prior to the addition of trieylamine (0.5 ml, 3.59 mmol) and 2c (328 mg, 1.16 mmol) in THF (2 ml). The solution was then stirred for 30 minutes at room temperature, diluted with brine, acidified to pH=1 with HCl, extracted with ethyl acetate. The organic layer was dried (Na₂SO₄), filtered, and evaporated. The residue was dissolved in acetone, and triturated with hexanes to afford 3 (204 mg, 35%) as a white solid. LRMS (ESI): (calc.) 495.6; (found) 496.2 (MH)⁺.

### Step 4: N-(Biphenyl-3-yl)-6-(sulfamoylamino)hexanamide (4)

To a solution of 3 (204 mg, 0.412 mmol) in MeOH (5 ml) was added 10% Pd/C (130 mg). The resulting mixture was stirred under hydrogen atmosphere for 1 hour, filtered through a pad of celite, and concentrated. The residue was take up in ethyl acetate, and triturated with hexanes to afford 4 (53 mg, 36%) as a light yellow solid. (MeOD-d4) δ (ppm) ¹H, 7.90-7.86 (m, 1H), 7.66-7.61 (m, 2H), 7.58-7.54 (m, 1H), 7.49-7.34 (m, 5H), 3.08 (t, J=7.0 Hz, 2H), 2.45 (t, J=7.4 Hz, 2H), 1.83-1.73 (m, 2H), 1.70-1.61 (m, 2H), 1.55-1.45 (m, 2H). LRMS (ESI): (calc) 361.5; (found) 362.2 (MH)⁺.

### Example 2a

### N-(Quinolin-6-yl)-6-(sulfamoylamino)hexanamide (5a)

### Step 1: tert-Butyl 6-oxo-6-(quinolin-6-ylamino)hexylcarbamate (1a)

To a solution of N-Boc caproic acid (0.23 g, 1.0 mmol) in DCM (5 ml) was added PS-carbodiiminde (0.8 g, 1.1 mmol). After 10 minutes 6-aminoquinoline (0.1 g, 0.7 mmol) and HOBt (0.13 g, 1.0 mmol) were added and stirred over night at 35° C. The mixture was filtered and concentrated. The residue was purified by silica gel column chromatography with gradient of EtOAc (20-100%) in Hexane to afford 1a (0.2 g, 56%) as a beige solid. LRMS (ESI): (calc.) 357.2; (found) 358.3 (MH)⁺.

### Step 2: 6-Amino-N-(quinolin-6-yl)hexanamide (2a)

Compound 1a (0.2 g, 0.56 mmol) was suspended in 4N HCl in dioxane (3 ml), stirred 3 h then concentrated. The residue was triturated with ethyl ether over night to afford 2a (0.164 g, quant.) as a beige solid. The solid was dissolved in DCM/MeOH (˜3 ml) and treated with MP-carbonate resine to afford the free base as an oils. LRMS (ESI): (calc.) 257.2; (found) 258.3 (MH)⁺.

### Step 3: N-(Quinolin-6-yl)-6-(sulfamoylamino)hexanamide (5a)

To a biphasic mixture of 2a (0.164 g, 0.56 mmol) in 10% Et₃N in toluene (2 ml) was added sulfamide (269 mg, 2.8 mmol). The mixture was heated to 130° C. in a sealed vial for 45 min then cooled to room temperature. HCl 1N (3 ml) was added and stirred 15 minutes. The mixture was basified with a saturated bicarbonate solution and extracted with ethyl acetate, washed with water, brine, dried (Na₂SO₄) filtered and concentrated. The residue was purified by silica gel column chromatography with gradient of EtOAc (20-100%) in hexanes then 10% MeOH in EtOAC to afford 5a (5 mg, 3%) as a white solid. (MeOD-d4) δ (ppm) ¹H, 8.76-8.70 (m, 1H), 8.37 (d, J=1.9 Hz, 1H), 8.26 (d, J=8.2 Hz, 1H), 7.95 (d, J=9.2 Hz, 1H), 7.78 (dd, J=9.1 and 2.3 Hz, 1H), 7.49 (dd, J=8.4 and 4.3 Hz, 1H), 3.05 (t, J=7.0 Hz, 2H), 2.46 (t, J=7.4 Hz, 2H), 1.82-1.72 (m, 2H), 1.68-1.58 (m, 2H), 1.54-1.43 (m, 2H). LRMS: 336.1 (calc), 335.0 (found).

### Example 2b

### N-Phenyl-6-(sulfamoylamino)hexanamide (5b)

### Step 1: tert-Butyl 6-oxo-6-(phenylamino)hexylcarbamate (1b)

To a stirred solution of N-Boc caproic acid (0.24 g, 1.1 mmol) in DCM (10 ml) was added 1-chloro-N,N,2-trimethylpropenylamine (0.14 g, 1.1 mmol) at 0° C. After 1.5 hour aniline (89 mg, 0.95 mmol) and triethylamine (114 mg, 1.43 mmol) were added and stirred for over night. The mixture was diluted with ethyl acetate and washed with 3N HCl, saturated bicarbonate solution and brine then, dried (Na₂SO₄), filtered and concentrated. The residue was purified by silica gel column chromatography with gradient of EtOAc (0-70%) in Hexane to afford 1b (67 mg, 23%) as an oil. LRMS: 306.2 (calc), 307.1 (found).

### Steps 2-3: N-Phenyl-6-(sulfamoylamino)hexanamide (5b)

The general Procedures E and F were followed to afford 5b (27 mg, 43%) as a beige solid. (MeOD-d4) δ (ppm) ¹H, 7.55-7.52 (m, 2H), 7.31-7.25 (m, 2H), 7.07 (t, J=7.4 Hz, 1H), 3.03 (t, J=7.0 Hz, 2H), 2.38 (t, J=7.5 Hz, 2H), 1.77-1.67 (m, 2H), 1.66-1.56 (m, 2H), 1.50-1.40 (m, 2H). LRMS: 285.11 (calc) 284.14 (found) (MH)⁺.

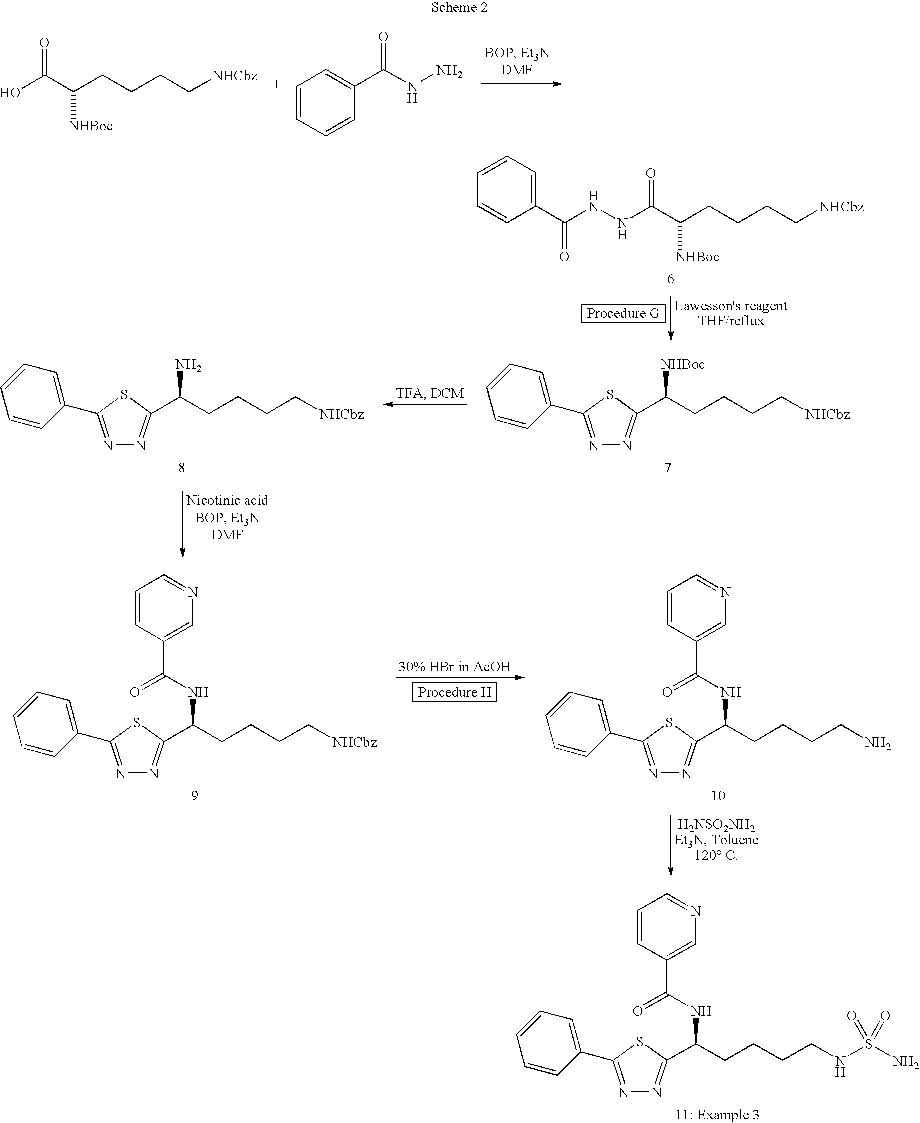

### Example 3

### (S)—N-(1-(5-Phenyl-1,3,4-thiadiazol-2-yl)-5-(sulfamoylamino)pentyl)nicotinamide (11)

### Step 1: (S)-Benzyl 5-(t-butylcarbamate)-6-(2-benzoylhydrazinyl)-6-oxohexylcarbamate (6)

The general Procedure A was followed to afford 6 (7.4 g, 80%) as a white solid. LRMS (ESI): (calc) 498.2 (found) 499.4 (MH)⁺.

### Step 2: (S)-Benzyl 5-(t-butylcarbamate)-5-(5-phenyl-1,3,4-thiadiazol-2-yl)pentylcarbamate (7)

To a stirred solution of 6 (4.60 g, 9.23 mmol) in THF (80 ml) at room temperature was added Lawesson's reagent (3.92 g, 9.69 mmol). The resulting solution was heated to 70° C. for 2 hours prior to cooling, removal of the solvent, and direct purification of the residue by silica gel column chromatography using EtOAc (30%) in hexanes to afford 7 (2.39 g, 46%) as a white solid. LRMS (ESI): (calc) 496.2 (found) 497.3 (MH)⁺.

### Step 3: (S)-Benzyl 5-amino-5-(5-phenyl-1,3,4-thiadiazol-2-yl)pentylcarbamate (8)

The general Procedure B was followed to afford 8 (2.39 g, 46%) as a white solid. LRMS (ESI): (calc) 496.2 (found) 497.3 (MH)⁺.

### Step 4: (S)-Benzyl 5-(nicotinamido)-5-(5-phenyl-1,3,4-thiadiazol-2-yl)pentylcarbamate (9)

The general Procedure A was followed to afford to afford 9 (256 mg, 92%) as a white foam. LRMS (ESI): (calc) 501.2 (found) 502.3 (MH)⁺.

### Step 5: (S)—N-(5-Amino-1-(5-phenyl-1,3,4-thiadiazol-2-yl)pentyl)nicotinamide (10)

Compound 9 (256 mg, 0.511 mmol) was dissolved in HBr 33% wt. in acetic acid (3.0 ml) and stirred 1 hour. Then the reaction mixture was concentrated and the orange solid obtained was washed with 30% ethyl acetate in hexanes. The solid was dissolved in water. A solution of 5% aqueous sodium hydroxide solution was added until pH=12. The product was extracted with DCM. The combined organic phases were dried (Na₂SO₄), filtered and evaporated to afford 10 (251 mg, quantitative) as a beige solid. LRMS (ESI): (calc) 367.1 (found) 368.3 (MH)⁺.

### Step 6: (S)—N-(1-(5-Phenyl-1,3,4-thiadiazol-2-yl)-5-(sulfamoylamino)pentyl)nicotinamide (11)

The general Procedure F was followed to afford 11 (89 mg, 21%) as a light yellow solid. (MeOD-d4) δ (ppm) ¹H, 9.07 (d, J=2.3 Hz, 1H), 8.75 (dd, J=4.9, 1.6 Hz, 1H), 8.37-8.32 (m, 1H), 8.02-7.97 (m, 2H), 7.64-7.52 (m, 4H), 5.65 (dd, J=9.0, 5.9 Hz, 1H), 3.11 (t, J=6.7 Hz, 2H), 2.40-2.18 (m, 2H), 1.80-1.58 (m, 4H). LRMS (ESI): (calc) 446.5; (found) 447.2 (MH)⁺.

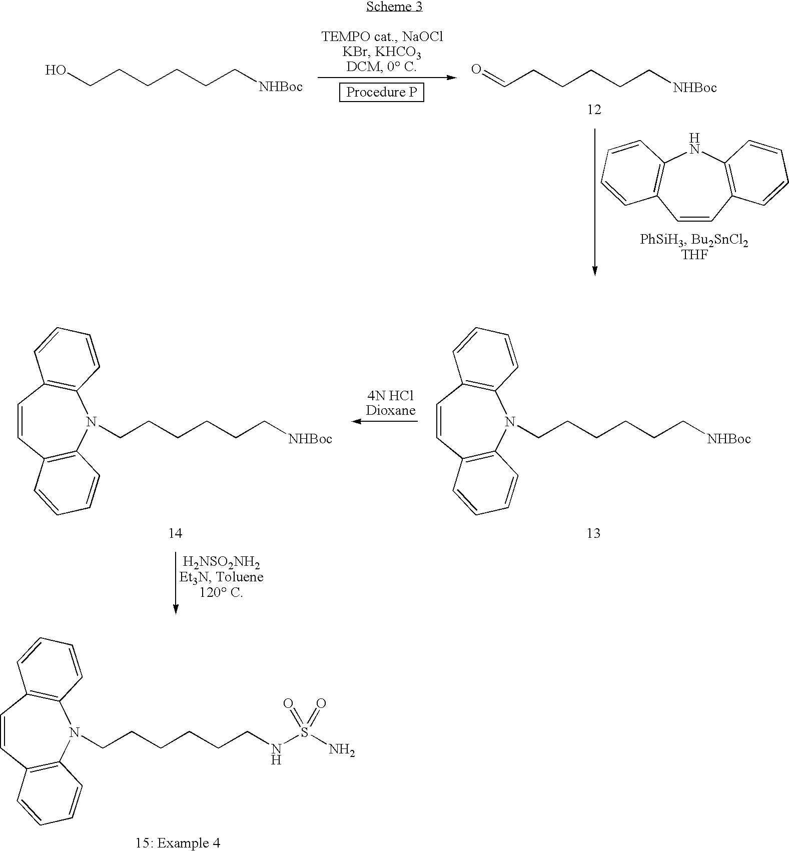

### Example 4

### (Z)-6-(5H-Dibenzo[b,f]azepin-5-yl)hexan-1-sulfamate (15)

### Step 1: tert-Butyl 6-oxohexylcarbamate (12)

To a stirred solution of 6-(Boc-amino)-hexanol (2.0 g, 9.2 mmol) and TEMPO (29 mg, 0.18 mmol) in DCM (20 ml) at 0° C. was added a 2.75 M KBr solution (7.3 ml) and a 1.6 M KHCO₃ solution (32 ml). Then a 5.8% NaOCl (Javex) solution (15.9 ml) was added dropwise and stirred for 1.2 hour. A saturated Na₂S₂O₃ solution was added and the mixture was extracted with DCM. The organic extract was dried (Na₂SO₄), filtered, and evaporated to afford 12 (2.0 g, quant.) as an oil. LRMS (ESI): (calc.) 215.2; (found) 216.2 (MH)⁺.

### Step 2: (Z)-tert-Butyl 6-(5H-dibenzo[b,f]azepin-5-yl)hexylcarbamate (13)

To a solution of iminostilbene (2.1 g, 9.2 mmol) in THF (10 ml) was added aldehyde 12 (2.0 g, 9.2 mmol), dibutyltin dichloride (0.93 g, 3 mmol) and triphenyl silane (2.0 g, 18.4 mmol). The resulting solution was stirred at room temperature over night and then evaporated. The residue was purified by silica gel column chromatography with gradient of EtOAc (0-50%) in Hexane to afford 13 (3.58 g, 99%) as an oil. LRMS (ESI): (calc.) 392.25; (found) 393.2 (MH)⁺.

### Step 3: (Z)-6-(5H-Dibenzo[b,f]azepin-5-yl)hexan-1-amine (14)

The general Procedure E was followed to afford to afford 14 (0.73 g, quant.) as a beige solid. LRMS (ESI): (calc.) 292.25; (found) 293.2 (MH)⁺.

### Step 4: (Z)-6-(5H-Dibenzo[b,f]azepin-5-yl)hexan-1-sulfamate (15)

The general Procedure F was followed to afford 15 (5 mg, 5%) as a white solid. (MeOD-d4) δ (ppm) ¹H, 7.27-7.20 (m, 2H), 7.06-7.01 (m, 6H), 6.95 (dt, J=7.3, 1.0 Hz, 2H), 6.70 (s, 2H), 3.71 (t, J=6.7 Hz, 2H), 2.94 (t, J=7.3 Hz, 2H), 1.58-1.35 (m, 6H), 1.33-1.21 (m, 2H). LRMS (ESI): (calc) 371.2 (found) 370.1 (MH)⁺.

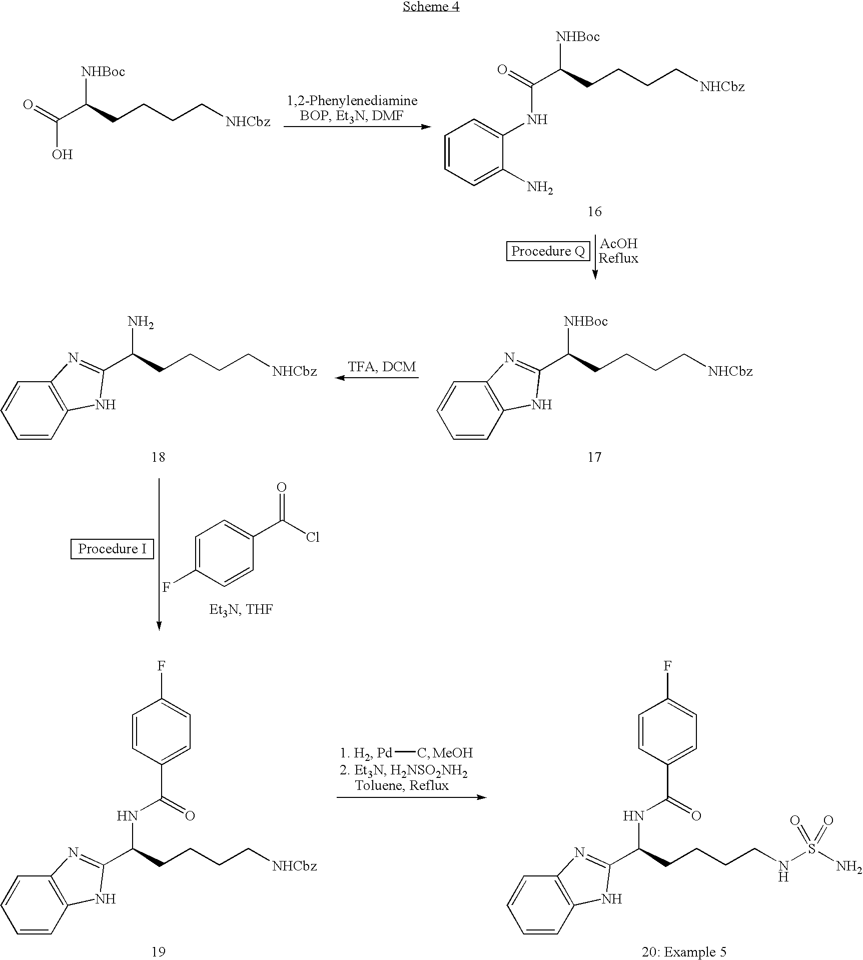

### Example 5

### (S)—N-(1-(1H-Benzo[d]imidazol-2-yl)-5-(sulfamoylamino)pentyl)-4-fluorobenzamide (20)

### Step 1: (S)-6-(Benzyloxycarbonylamino)-2-(tert-butoxycarbonylamino)hexanoic-2-aminobenzamide (16)

The general Procedure A was followed to afford 16 (2.1g, 68%) as a yellow oil. LRMS (ESI): (calc.) 470.2; (found) 471.2 (MH)⁺.

### Step 2: (S)-Benzyl 5-(tert-butoxycarbonylamino)-5-(1H-benzo[d]imidazol-2-yl)pentylcarbamate (17)

Acetic acid (6 ml) was added to 16 (1.55 g, 3.3 mmol) and heated at 90° C. for 45 minutes. The solvent was evaporated under reduced pressure. The residue was then purified by silica gel column chromatography with gradient of EtOAc (20-100%) in hexane to afford 17 (1.77 g, 88%) as a light yellow oil. LRMS (ESI): (calc.) 452.2; (found) 453.8 (MH)⁺.

### Step 3: (S)-Benzyl 5-amino-5-(1H-benzo[d]imidazol-2-yl)pentylcarbamate (18)

The general Procedure B was followed to afford 18 (1.32 g, 96%) as a yellow oil. LRMS (ESI): (calc.) 352.2; (found) 353.1 (MH)⁺.

### Step 4: (S)-Benzyl 5-(1H-benzo[d]imidazol-2-yl)-5-(4-fluorobenzamido)pentylcarbamate (19)

To a solution of 18 (535 mg) 1.52 mmol) in THF (3 ml) was added 4-fluorobenzoyl chloride (0.18 ml, 1.52 mmol) and Et₃N (0.4 ml). After stirring at room temperature for 30 min, the solution was diluted with brine, extracted with EtOAc. The organic extract was dried (Na₂SO₄), filtered, and evaporated. The residue was purified by silica gel column chromatography with gradient of EtOAc (20-100%) in Hexane to afford 19 (281 mg, 40%) as a deep yellow oil. LRMS (ESI): (calc.) 474.2; (found) 475.6 (MNa)⁺.

### Steps 5-6: (S)—N-(1-(1H-Benzo[d]imidazol-2-yl)-5-(sulfamoylamino)pentyl)-4-fluoro-benzamide (20)

The general Procedures C and F were followed to afford 20 (11 mg 21%) as white solid. (MeOD-d4) δ (ppm) ¹H, 7.98 (q, J=5.4, 8.8 Hz, 1H), 7.53 (bs, 2H), 7.2 (m, 4H), 5.39 (q, J=5.9, 8.8 Hz, 1H), 3.04 (t, J=6.6 Hz, 2H), 2.22 (m, 1H), 2.11 (m, 1H), 1.69-1.49 (m, 4H). LRMS: (calc.) 419.1; (found) 419.8 (MH)⁺.

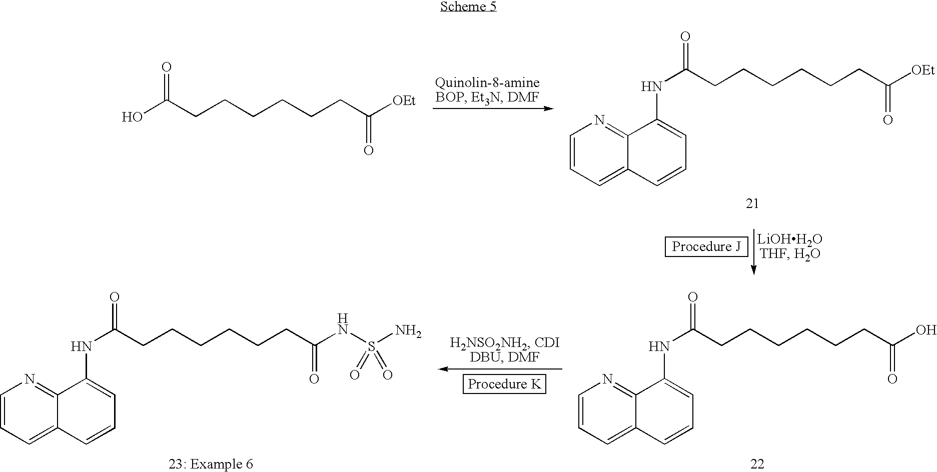

### Example 6

### N¹-(Quinolin-8-yl)-N⁸-sulfamoyloctanediamide (23)

### Step 1: Ethyl 8-oxo-8-(quinolin-8-ylamino)octanoate (21)

The general procedure A was followed to afford 21 (1.4 g, 56%) as a yellow solid LRMS (ESI): (calc) 328.1 (found) 329.0 (MH)⁺.

### Step 2: 8-Oxo-8-(quinolin-8-ylamino)octanoic acid (22)

To a solution of methyl ester 21 (500 mg, 1.52 mmol) in THF: water solvent mixture (2 ml), was added lithium hydroxide monohydrate (0.11 mg, 4.57 mmol). The reaction stirred vigorously for 1 hour, acidified with 1M HCl solution and the solvent evaporated under reduced pressure. The residue was purified by silica gel column chromatography with a gradient of MeOH (0-30%) in CH₂Cl₂ to afford 22 (210 mg, 46%) as a white solid. LRMS (ESI): (calc) 300.0 (found) 301.1 (MH)⁺.

### Step 3: N¹-(Quinolin-8-yl)-N⁸-sulfamoyloctanediamide (23)

CDI (45 mg, 0.28 mmol) was added to a solution of 22 (42 mg, 0.14 mmol) in DMF (1.5 ml) and the reaction stirred at room temperature for 1.5 h. The sulfamide (40 mg, 0.42 mmol) was then added to the reaction mixture followed by dropwise addition of DBU (0.063 ml, 0.42 mmol). The reaction was then stirred for an additional 15 min. The solvent was then evaporated reduced pressure and the residue was purified by silica gel column chromatography with a gradient of MeOH (0-5%) in CH₂Cl₂ to afford 23 (23 mg, 44%) as a white solid. (DMSO-d6) δ (ppm) ¹H: 11.33 (s, 1H), 10.04 (s, 1H), 8.91 (m, 1H), 8.60 (d, J=7.0 Hz, 1H), 8.39 (d, J=8.2 HZ, 1H), 7.63 (m, 2H), 7.55 (t, J=7.8 Hz, 1H) 7.28 (bs, 2H), 2.57 (t, J=6.6 Hz, 2H), 2.22 (t, J=7.2 Hz, 2H), 1.61 (m, 4H). LRMS (ESI): (calc) 378.1 (found) 379.1 (MH)⁺.
Scheme 6

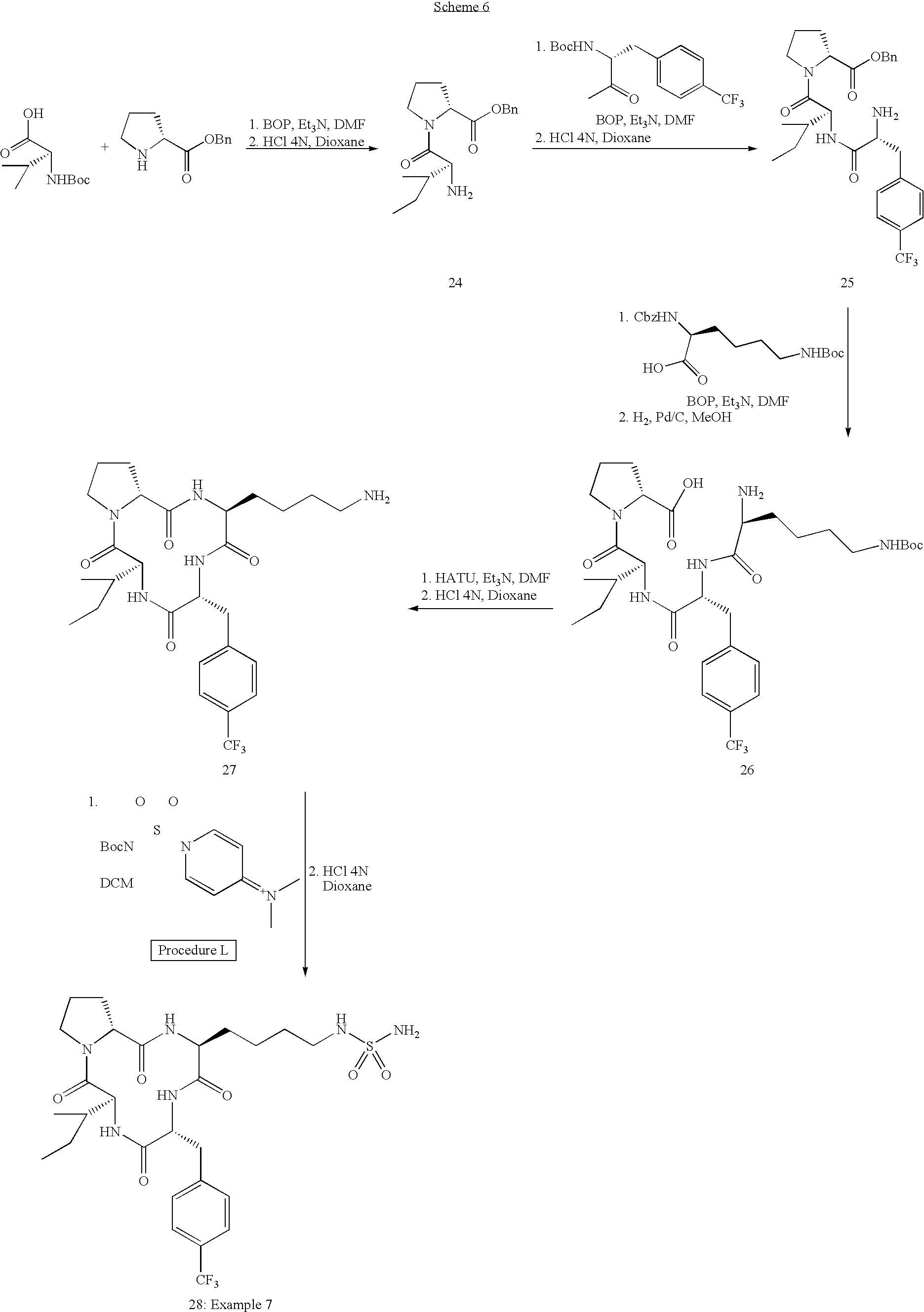

### Example 7

### N-(4-((3S,6R,9S,14aR)-9-sec-Butyl-1,4,7,10-tetraoxo-6-(4-(trifluoromethyl)benzyl)-tetradecahydropyrrolo[1,2-a][1,4,7,10]tetraazacyclododecin-3-yl)butyl)-sulfamide (28)

### Steps 1-6: (R)-1-((10S,13R,16S)-10-Amino-16-sec-butyl-2,2-dimethyl-4,11,14-trioxo-13-(4-(trifluoromethyl)benzyl)-3-oxa-5,12,15-triazaheptadecane)pyrrolidine-2-carboxylic acid (26)

General procedures A, E, A, E, A and C were followed to afford 26 (67 mg, 36%) as a white solid. LRMS (ESI): (calc.) 671.3; (found) 672.2 (MH)⁺.

### Steps 7-8: (3S,6R,9S,14aR)-3-(4-Aminobutyl)-9-sec-butyl-6-(4-(trifluoromethyl)benzyl)-decahydropyrrolo[1,2-a][1,4,7,10]tetraazacyclododecine-1,4,7,10-tetraone (27)

A solution of HATU (720 mg, 3.8 mmol) in DMF (35 ml) was added to a solution of 421 mg, 0.627 mmol) and Et₃N (870 μL) in DMF (15 ml).The reaction was stirred for 3 h, and then partitioned between EtOAc and H₂O. The organic phase was separated, dried (Na₂SO₄), filtered and concentrated under reduced pressure. The residue was purified by trituration with Et₂O to afford to afford tert-butyl 4-((3S,6R,9S,14aR)-9-sec-butyl-1,4,7,10-tetraoxo-6-(4-(trifluoromethyl)benzyl)-tetradecahydropyrrolo[1,2-a][1,4,7,10]-tetraazacyclododecin-3-yl)butylcarbamate (107 mg, 26%) as a white solid. LRMS (ESI): (calc.) 653.3; (found) 654.4 (MH)⁺. The general Procedure E was then followed to afford 27 (87 mg, 96%) as a white solid. LRMS (ESI): (calc.) 533.2; (found) 534.2 (MH)⁺.

### Steps 9-10: N-(4-((3S,6R,9S,14aR)-9-sec-Butyl-1,4,7,10-tetraoxo-6-(4-(trifluoromethyl)benzyl)-tetradecahydropyrrolo[1,2-a][1,4,7,10]tetraazacyclododecin-3-yl)butyl)-sulfamide (28)

N-(tert-butoxycarbonyl)-N-[4-(dimethylazaniumylidene)-1,4-dihydropyridin-1-ylsulfonyl]-azanide (47 mg, 0.16 mmol) was added to a solution of 27 (87 mg, 0.16 mmol) in DCM and the reaction stirred overnight. The solvent was then evaporated under reduced pressure. The residue was purified by silica gel column chromatography with a gradient of EtOAc (10-100%) in Hexane to afford tert-butyl N-(4-((3S,6R,9S,14aR)-9-sec-butyl-1,4,7,10-tetraoxo-6-(4-(trifluoromethyl)benzyl)tetradecahydropyrrolo[1,2-a][1,4,7,10]tetra-azacyclododecin-3-yl)butyl)sulfamoylcarbamate (49 mg, 43%) as a white solid. LRMS (ESI): (calc.) 732.3; (found) 755.5 (MNa)⁺. The general Procedure E was then followed to afford 28 (17 mg, 40%) as a white solid. (MeOD-d₄) δ (ppm) ¹H, 7.56 (d, J=8 Hz, 2H) 7.43 (d, J=8.4 Hz, 2H), 4.81 (d, J=7.6 HZ, 1H), 4.75 (t, J=7.6 Hz, 1H), 4.45 (d, J=10.8 Hz, 1H), 4.30 (d, J=7.6 Hz, 1H), 3.98 (td, J=3.6, 9.6 Hz, 1H), 3.57 (q, J=8.2 Hz, 1H), 3.21 (dd, J=7.6, 14 Hz, 1H) 2.96 (m, 3H), 2.34 (m, 1H), 2.19 (m, 1H), 2.00 (m, 1H), 1.88 (m, 2H), 1.62 (m, 5H), 1.28 (m, 2H), 1.12 (m, 1H), 0.85 (m, 6H), LRMS; 632.2 (calc) 631.5 (found) (MH)⁺.

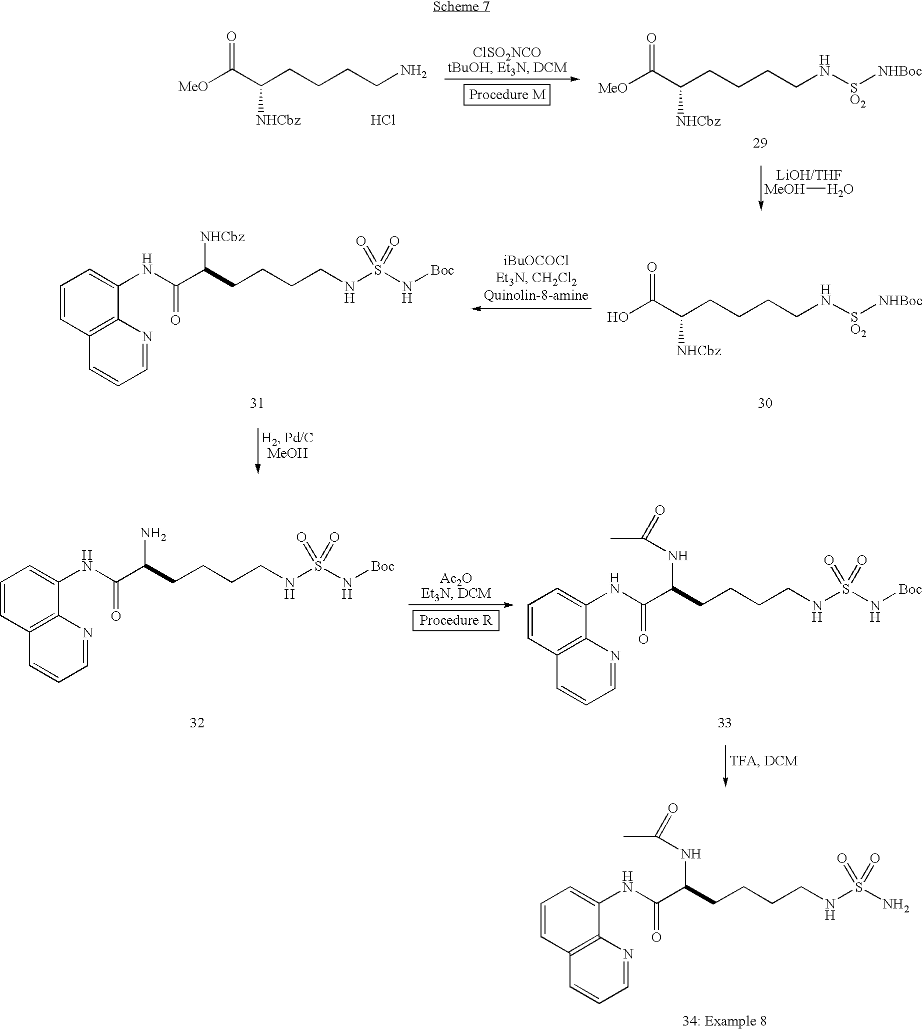

### Example 8

### (S)-2-Acetamido-N-(quinolin-8-yl)-6-(sulfamoylamino)hexanamide (34)

### Step 1: (S)-Methyl 2-(benzyloxycarbonylamino)-6-(N-(tert-butoxycarbonyl) sulfamoylamino)hexanoate (29)

To a stirred solution of chlorosulfonyl isocyanate (0.27 ml, 3.14 mmol)) in dichloromethane (4 ml) at 0° C. was added 2-methyl-2-propanol (0.30 ml, 3.14 mmol). The reaction was stirred at room temperature for 30 min. The mixture was added to a stirred solution of (S)-5-(benzyloxycarbonylamino)-6-methoxy-6-oxohexan-1-aminium chloride (1.079 g, 3.27 mmol) and triethylamine (1.31 ml, 9.41 mmol) in dichloromethane (4 ml) at 0° C. The reaction was stirred at room temperature for 1 h. The solvent was evaporated, water added and the aqueous phase was extracted with ethyl acetate. The organic extract was dried (MgSO₄), filtered, and evaporated. The residue was purified by silica gel column chromatography with gradient of methanol (0-5%) in dichloromethane to afford 29 (1.098 g, 74%) as a white solid. (DMSO-d₆) δ (ppm) ¹H, 10.76 (s, 1H), 7.71 (d, J=7.6 Hz, 1H), 7.51 (m, 1H), 7.38-7.26 (m, 5H), 5.01 (s, 2H), 3.97 (q, J=3.9 Hz, 1H), 3.61 (s, 3H), 2.82 (q, J=6.3 Hz, 2H), 1.68-1.50 (m, 2H), 1.43-1.22 (m, 4H), 1.40 (s, 9H). LRMS (ESI): (calc) 473.2; (found) 496.3 (M+Na)⁺.

### Step 2: (S)-2-(Benzyloxycarbonylamino)-6-(N-(tert-butoxycarbonyl)sulfamoylamino)hexanoic acid (30)

The general procedure J was followed to afford 30 (676 mg, quantitative). LRMS (ESI): (calc) 465.2; (found) 466.3 (MH)⁺.

### Step 3: (S)-Benzyl 1-oxo-1-(quinolin-8-ylamino)-6-(N-(tert-butoxycarbonyl)-sulfamoyl-amino)hexan-2-ylcarbamate (31)

To a stirred solution of the crude acid 30 (1.82 g, 3.98 mmol) in CH₂Cl₂ (15 ml) at 0° C. were added isobutyl chloroformate (0.57 ml, 4.39 mmol) and Et₃N (0.61 ml, 4.39 mmol). After 30 min, a solution of 8-aminoquinoline (633 mg, 4.39 mmol) and Et₃N (0.61 ml, 4.39 mmol) in CH₂Cl₂ (5 ml) was added drop-wise. The mixture was stirred at room temperature for 2 h and H₂O was added and the aqueous phase was extracted with CH₂Cl₂ and the organic extracts were combined and dried (Na₂SO₄). The residue was then purified by silica gel column chromatography with gradient of EtOAc (40-80%) in hexane to afford 31 (1.63 g, 70%) as a light yellow oil. (MeOD-d₄) δ (ppm) ¹H, 8.77 (d, J=4.0 Hz, 1H), 8.65 (d, J=7.2 Hz, 1H), 8.30 (d, J=8.0 Hz, 1H), 7.63 (d, J=8.0 Hz, 1H), 7.57-7.53 (m, 2H), 7.39 (d, J=6.4 Hz, 2H), 7.27 (d, J=6.4 Hz, 2H), 5.23-5.10 (m, 2H), 4.34 (q, J=4.8 Hz, 1H), 3.02 (t, J=6.4 Hz, 2H), 2.04-2.00 (M, 1H), 1.81-1.77 (m, 1H), 1.63-1.48 (m, 4H), 1.45 (s, 9H). LRMS (ESI): (calc) 585.7; (found) 586.5 (MH)⁺.

### Step 4: (S)-tert-Butyl N-(5-amino-6-oxo-6-(quinolin-8-ylamino)hexyl)sulfamoylcarbamate (32)

The general procedure C was followed to afford 32 (1.21 g, 96%) as a white solid. LRMS (ESI): (calc) 451.5; (found) 452.5 (M+Na)⁺.

### Step 5: (S)-tert-Butyl N-(5-acetamido-6-oxo-6-(quinolin-8-ylamino)hexyl)-sulfamoyl-carbamate (33)

To a stirred solution of 32 (206 mg, 0.46 mmol) in CH₂Cl₂ (2.5 ml) were added Et₃N (0.14 ml, 1.00 mmol) and Ac₂O (0.05 ml, 0.50 mmol). After stirring for 1 h, 10% HCl solution was added and the aqueous phase was extracted three times with DCM. The organic extracts were combined, dried over Na₂SO₄ and evaporated. The resulting residue was purified by silica gel column chromatography with gradient of EtOAc (60-100%) in hexane to afford 33 (184 mg, 82%) as a colorless oil. (MeOD-d4) δ (ppm): 8.86 (dd, J=1.6, 4.4 Hz, 1H), 8.62 (d, J=7.6 Hz, 1H), 8.29 (dd, J=1.6, 8.4 Hz, 1H), 7.62 (d, J=8.4 Hz, 1H), 7.56-7.52 (m, 2H), 4.57 (q, J=4.8 Hz, 1H), 3.03 (t, J=6.8 Hz, 2H), 2.12 (s, 3H), 2.04-1.79 (m, 1H), 1.87-1.79 (m, 1H), 1.65-1.49 (m, 4H), 1.45 (s, 9H). LRMS (ESI): (calc.) 493.2; (found) 494.5 (MH)⁺.

### Step 6: (S)-2-Acetamido-N-(quinolin-8-yl)-6-(sulfamoylamino)hexanamide (34)

The general procedure B was followed to afford 34 (95 mg, 66%) as a white foam. (MeOD-d4) δ (ppm): 8.67 (dd, J=1.6, 4.4 Hz, 1H), 8.62 (d, J=7.6 Hz, 1H), 8.30 (dd, J=1.6, 8.4 Hz, 1H), 7.63 (d, J=8.4 Hz, 1H), 7.57-7.53 (m, 2H), 4.57 (q, J=4.8 Hz, 1H), 3.05 (t, J=6.8 Hz, 2H), 2.12 (s, 3H), 2.09-1.97 (m, 1H), 1.82-1.79 (m, 1H), 1.64-1.55 (m, 4H). LRMS: 393.5 (calc) 394.4 (found) (MH)⁺.

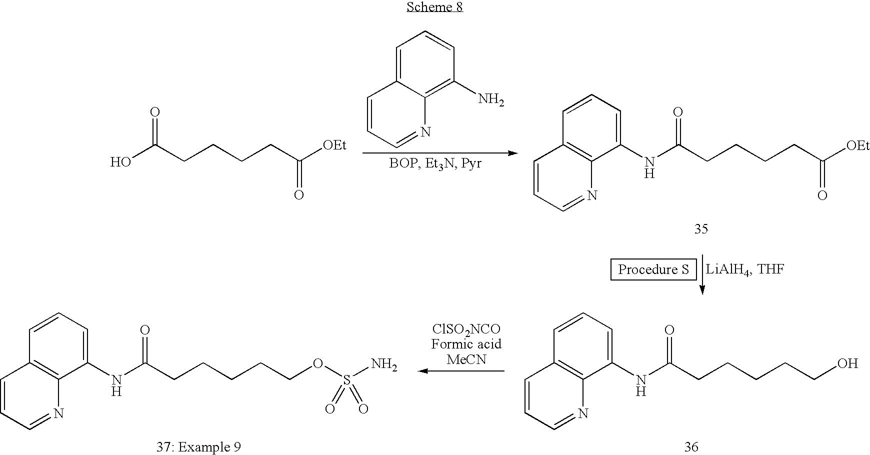

### Example 9

### 6-Oxo-6-(quinolin-8-ylamino)hexyl sulfamate (37)

### Step 1: Ethyl 6-oxo-6-(quinolin-8-ylamino)hexanoate (35)

The general Procedure A was followed to afford 35 (2.24 g, 72%) as a light yellow oil. LRMS (ESI): (calc) 300.3 (found) 301.2 (MH)⁺.

### Step 2: 6-Hydroxy-N-(quinolin-8-yl)hexanamide (36)

To a solution of 35 (1.12 g, 3.73 mmol) in THF (10 ml) was added LiAH₄ (425 mg, 11.2 mmol). The mixture was stirred for 15 minutes at 0° C. The mixture was quenched with aqueous solution of sodium sulfate (ss), filtered through a pad of celite and extracted with DCM. The organic extract was dried (Na₂SO₄), filtered, and evaporated. The residue was purified by silica gel column chromatography with ethyl acetate (0-100%) in hexanes to afford 36 (346 mg, 36%) as a light yellow oil. LRMS (ESI): (calc.) 258.2; (found) 259.2 (MH)⁺.

### Step 3: 6-Oxo-6-(quinolin-8-ylamino)hexyl sulfamate (37)

The general Procedure M was followed to afford 37 (35 mg, 8%) as a white solid. (DMSO-d6) δ (ppm) ¹H, 10.11 (s, 1H), 8.97 (d, J=2.7 Hz, 1H), 8.67 (d, J=7.2 Hz, 1H), 8.44 (d, J=8.2 Hz, 1H), 7.74-7.57 (m, 3H), 7.46 (s, 2H), 4.07 (t, J=6.1 Hz, 2H), 2.64 (t, J=7.4 Hz, 2H), 1.79-1.67 (m, 4H), 1.52-1.41 (m, 2H). LRMS (ESI): (calc) 337.4; (found) 338.1 (MH)⁺.

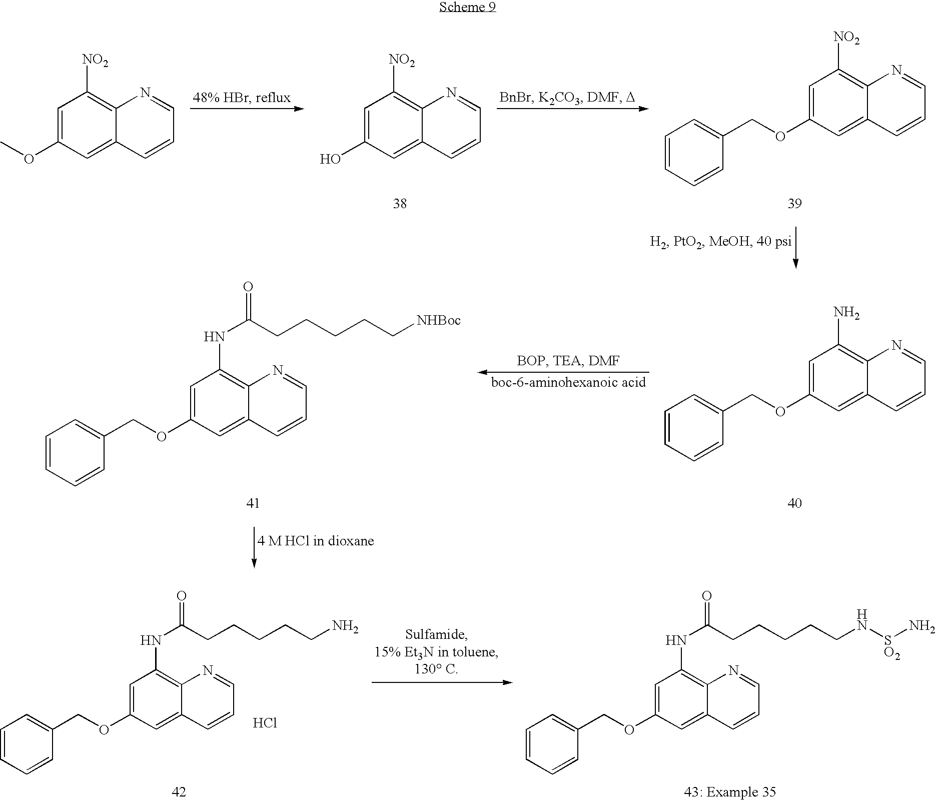

### Example 35

### N-(6-(benzyloxy)quinolin-8-yl)-6-(sulfamoylamino)hexanamide (43)

### Step 1: 8-nitroquinolin-6-ol (38)

6-methoxy-8-nitroquinoline (2.15 g, 10.51 mmol) was dissolved in 48% HBr (8.0 mL, 147.20 mmol) and refluxed for 16 hours. The resulting suspension of yellow crystals was cooled down and then filtered. The solid was diluted in water and 15% NaOH (aq) was added. The suspension was filtered and the aqueous layer was acidified to obtain crystals at pH˜6. Crystals were washed with water and filtered to afford compound 38 as yellow crystals (1.89 g, 95%). LRMS (ESI): (calc.) 190.04 (found) 191.1 (MH)⁺.

### Step 2: 6-(benzyloxy)-8-nitroquinoline (39)

Benzyl bromide (0.55 ml, 4.65 mmol) was added to a suspension of K₂CO₃ (2.14 g, 15.52 mmol) and compound 38 (0.59 g, 3.10 mmol) in DMF (20 mL) and stirred at 50° C. for 20 hours. The resulting mixture was cooled down and diluted with EtOAc. The organic layer was washed with water, brine, dried over Na₂SO₄ and evaporated under vacuum. The residue was purified by silica gel column chromatography (0-50% EtOAc/Hexanes) to afford 39 as light yellow crystals (832.0 mg, 96%). LRMS (ESI): (calc.) 280.1 (found) 281.2 (MH)⁺.

### Step 3: 6-(benzyloxy)quinolin-8-amine (40)

PtO₂ (27.0 mg, 0.12 mmol) was added to a solution of compound 39 (0.83 g, 2.97 mmol), in methanol (15 ml) and THF (3 ml) and shaked under H₂ atmosfer at 40 psi in a Parr equipment for 30 minutes. The resulting mixture was diluted with methanol and filtered through a nylon membrane. The solvents were evaporated under vacuum and the product was isolated by silica gel column chromatography (0-75% EtOAc/Hexanes) to afford 40 as an amber oil (499 mg, 67%). LRMS (ESI): (calc.) 250.1 (found) 251.2 (MH)⁺.

### Step 4: tert-butyl 6-(6-(benzyloxy)quinolin-8-ylamino)-6-oxohexylcarbamate (41)

Triethylamine (0.11 ml, 0.81 mmol) was added to a solution of boc-6-aminohexanoic acid (63 mg, 0.27 mmol), compound 40 (71 mg, 0.28 mmol) and BOP (0.13 g, 0.28 mmol) in DMF (2 ml) and stirred at room temperature for 56 hours. Additional Boc-6-aminohexanoic acid (63 mg, 0.27 mmol) and BOP (0.13 g, 0.28 mmol) was added stirred at room temperature for 18 hours. The resulting mixture was diluted with EtOAc and the organic layer was washed with water, brine, dried over Na₂SO₄ and evaporated under vacuum. The residue was purified by silica gel column chromatography (0-50% EtOAc/Hexanes) to afford 41 as a pink oil (76 mg, 61%). LRMS (ESI): (calc.) 463.6 (found) 464.5 (MH)+408.4 (M-tBu).

### Step 5: 6-amino-N-(6-(benzyloxy)quinolin-8-yl)hexanamide hydrochloride (42)

Compound 41 (0.31 g, 0.67 mmol) was dissolved in 4M HCl in dioxane (5 mL) and stirred for 1 hour. The solvents were evaporated under vacuum. The residue was triturated in ether and filtered to afford 42 as a light beige solid (257 mg, 96%). LRMS (ESI): (calc.) 363.2 (found) 364.3 (MH)⁺.

### Step 6: N-(6-(benzyloxy)quinolin-8-yl)-6-(sulfamoylamino)hexanamide (43)

Amine 42 (0.10 g, 0.25 mmol) was added to a solution of sulfamide (0.12 g, 1.25 mmol) in 15% triethylamine in toluene solution (4.6 mL) and stirred at 130° C. for 20 minutes. The resulting mixture was cooled down and the solvents were evaporated under vacuum. The residue was dissolved in 3N HCl and stirred for 30 minutes, then neutralized to pH˜7 with a saturated solution of NaHCO₃ and extracted with EtOAc. The combined organic extracts were washed with brine, dried over Na₂SO₄ and evaporated under vacuum. The residue was purified by silica gel column chromatography (50-75% EtOAc-Hexanes) to afford 43 as white crystals (11 mg, 10%). ¹H NMR (MeOD-d4) d(ppm): 8.70 (d, J=4.3 Hz, 1H), 8.43 (d, J=2.5 Hz, 1H), 8.17 (d, J=8.4 Hz, 1H), 7.51-7.33 (m, 6H), 7.07 (d, J=2.4 Hz, 1H), 5.22 (s, 2H), 3.05 (t, J=7.0 Hz, 2H), 2.62 (t, J=7.4 Hz, 2H), 1.83-1.52 (m, 6H). LRMS (ESI): (calc.) 442.2 (found) 441.1 (M-H).

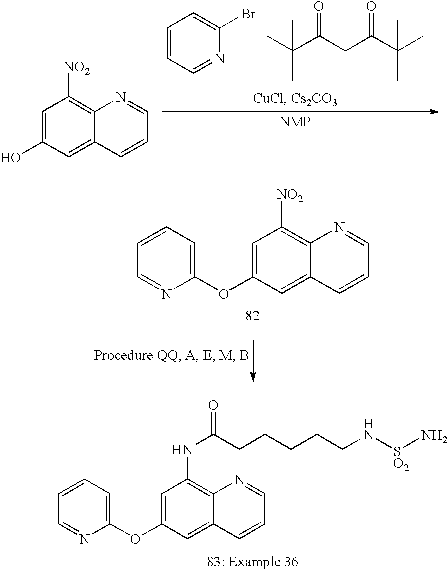

### Example 36

### N-(6-(pyridin-2-yloxy)quinolin-8-yl)-6-(sulfamoylamino)hexanamide (93)

### Step 1: 8-nitro-6-(pyridin-2-yloxy)quinoline (82)

NMP (4 mL) was added to a mixture of 2-bromopyridine (187 ul, 1.96 mmol), 8-nitroquinolin-6-ol (0.75 g, 3.92 mmol), 2,2,6,6-tetramethylheptane-3,5-dione (102 ul, 0.49 mmol) and cesium carbonate (1.28 g, 3.92 mmol) under nitrogen atmosphere. CuCl (97 mg, 0.98 mmol) was added to the reaction mixture and stirred at 130° C. for 1.5 hours. The crude was diluted in EtOAc, washed with 1N HCl, 15% NaOH (aq), NaHCO₃ (ss), dried over Na₂SO₄ and evaporated under vacuum. The desired product was purified by silica gel column chromatography (0-100% EtOAc/Hexanes) to afford (82) as a yellow oil (433 mg, 83%). LRMS (ESI): (calc.) 267.1 (found) 268.2 (MH)+.

### Step 2-6: N-(6-(pyridin-2-yloxy)quinolin-8-yl)-6-(sulfamoylamino)hexanamide (83)

Using procedure QQ, A, E, M and B from compounds (82) the title compound (83) was obtained (50 mg, 67%). ¹H NMR (MeOD-d4) d(ppm): 8.84 (dd, J=4.1, 1.4 Hz, 1H), 8.49 (d, J=2.5 Hz, 1H), 8.26 (dd, J=8.4, 1.4 Hz, 1H), 8.16 (dd, J=5.1, 1.6 Hz, 1H), 7.91-7.86 (m, 1H), 7.56 (q, J=4.3 Hz, 1H), 7.31 (d, J=2.5 Hz, 1H), 7.19-7.16 (m, 1H), 7.08 (d, J=8.4 Hz, 1H), 3.04 (t, J=7.0 Hz, 2H), 2.62 (t, J=7.4 Hz, 2H), 1.82-1.75 (m, 2H), 1.65-1.59 (m, 2H), 1.53-1.48 (m, 2H). LRMS (ESI): (calc.) 429.2 (found) 430.3 (MH)+.

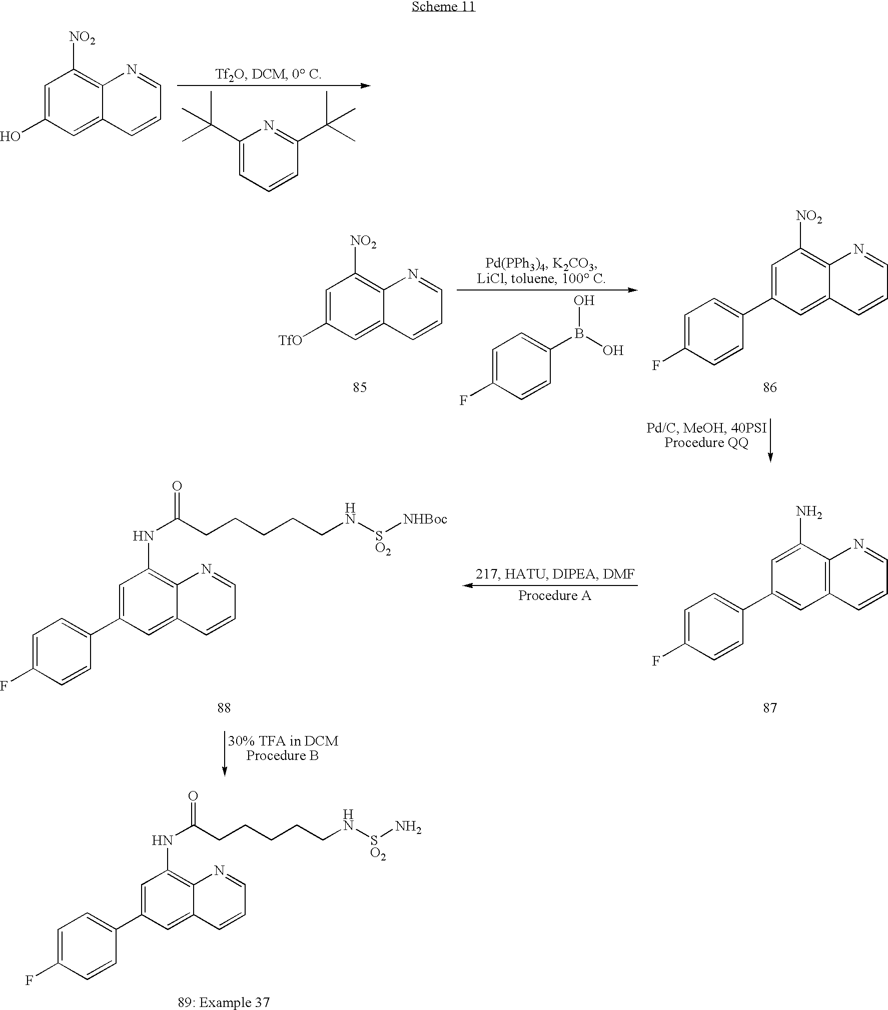

### Example 37

### N-(6-(4-fluorophenyl)quinolin-8-yl)-6-(sulfamoylamino)hexanamide (89)

### Step 1: 8-nitroquinolin-6-yl trifluoromethanesulfonate (85)

2,6-di-tert-butyl-4-methylpyridine (2.27 g, 11.04 mmol) was added to a suspension of 8-nitroquinolin-6-ol (1.40 g, 7.36 mmol) in DCM (20 ml) and cool down at 0° C. Then Tf₂O (1.49 ml, 8.83 mmol) in DCM (20 ml) was added at 0° C. and stirred at room temperature for 16 hours. A saturated solution of sodium bicarbonate was added to the reaction mixture, stirred for 15 minutes, extracted with DCM, dried over Na₂SO₄ and concentrated under vacuum. The residue was purified by silica gel column chromatography (0-100% EtOAc:Hexanes) to afford compound 85 a yellow oil (1.66 g, 70% yield). LRMS (ESI): (calc.) 322.0 (found) 323.1 (MH)+.

### Step 2: 6-(4-fluorophenyl)-8-nitroquinoline (86)

To a mixture of 8-nitroquinolin-6-yl trifluoromethanesulfonate (0.10 g, 0.31 mmol), 4-fluorobenzeneboronic acid (87 mg, 0.62 mmol), K₂CO₃ (0.06 g, 0.47 mmol), LiCl (15 mg, 0.34 mmol) was added 2 mL of toluene under nitrogen atmosphere. Pd(PPh₃)₄ (54 mg, 0.34 mmol) was added and stirred at 90° C. for 24 hours. The reaction mixture was cooled down to room temperature, diluted with EtOAc, washed with saturated Na₂CO₃, water and brine. The organic layer was dried over Na₂SO₄, and concentrated under vacuum. The residue was purified by silica gel column chromatography (0-35% EtOAc/Hexanes) to afford compound 86 a purple solid (73 mg, 94% yield). LRMS (ESI): (calc.) 268.1 (found) 269.2 (MH)+.

Step 3-5: N-(6-(4-fluorophenyl)quinolin-8-yl)-6-(sulfamoylamino)hexanamide (89)

Using procedure QQ, A and B with compound (86) the title compound (89) was obtained (36.5 mg, 47%). ¹H NMR (MeOD-d4) d(ppm): 8.97 (d, J=1.8 Hz, 1H), 8.87 (dd, J=4.3, 1.6 Hz, 1H), 8.37 (dd, J=8.3, 1.5 Hz, 1H), 7.84 (d, J=2.0 Hz, 1H), 7.79 (td, J=5.3, 2.2 Hz, 2H), 7.58 (q, J=4.2 Hz, 1H), 7.24 (t, J=8.7 Hz, 2H), 3.06 (t, J=6.9 Hz, 2H), 2.66 (t, J=7.4 Hz, 2H), 1.86-1.82 (m, 2H), 1.68-1.64 (m, 2H), 1.55-1.51 (m, 2H). LRMS (ESI): (calc.) 430.2 (found) 430.3 (MH)+.

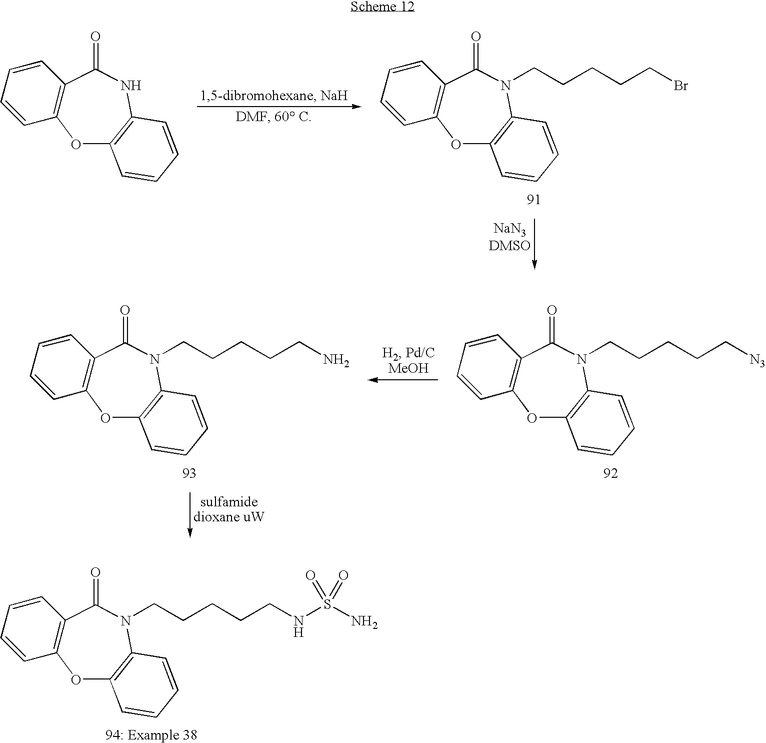

### Example 38

### Step 1: 10-(5-bromopentyl)dibenzo[b,f][1,4]oxazepin-11(10H)-one (91)

60% NaH in hexanes (1.25 equiv, 120 mg, 2.9 mmol) was added to a solution of dibenzo[b,f][1,4]oxazepin-11(10H)-one (1 equiv, 500 mg, 2.4 mmol) in DMF (6 mL) and stirred for 20 min. 1,5-dibromohexane (10 equiv, 5.4 g, 23.7 mmol) was then added and the reaction heated at 60° C. for 2 h. The mixture was cooled and then partitioned between water (15 mL) and Et₂O (5 mL). The organic layer was separated, dried over Na₂SO₄, filtered and concentrated. The residue was then purified by silica gel column chromatography eluting with 0-50% EtOAc in hexanes to afford 678 mg of compound 91 as a translucent oil (yield=79%). LRMS (ESI): (calc) 359.0 (found) 361.1H+

### Step 2: 10-(5-azidopentyl)dibenzo[b,f][1,4]oxazepin-11(10H)-one (92)

A 0.5 M solution of NaN₃ in DMSO (1.5 equiv, 6 mL, 2.8 mmol) was added to compound 91 (1 equiv, 678 mg, 1.9 mmol) and stirred overnight. The reaction was partitioned between EtOAc (4 mL) and H₂O (10 mL). The organic layer was separated, dried over Na₂SO₄, filtered and concentrated under reduced pressure to afford 527 mg of compound 92 as a clear translucent oil (yield=87%) LRMS (ESI): (calc) 322.1 (found) 323.2H+

### Step 3: 10-(5-aminopentyl)dibenzo[b,f][1,4]oxazepin-11(10H)-one (93)

Using procedure C compound 93 was afforded as a pale yellow oil 479 mg, (yield=99%). LRMS (ESI): (calc) 296.1 (found) 297.8H+

### Step 4: 10-(5-sulfamoylaminopentyl)dibenzo[b,f][1,4]oxazepin-11 (10H)-one (94)

In a 15 mL pressure vial, sulfamide (10 equiv, 486 mg, 5 mmol) and compound 93 (1 equiv, 150 mg, 0.5 mmol) were dissolved in 1 mL of dioxane. Once the sulfamide was completely dissolved the pressure vial was placed in the microwave and irradiated for 6 min. The reaction vial was cooled to 23° C. and the partitioned between EtOAc (2 mL) and water 2 mL). The organic phase was then separated, dried over Na₂SO₄, filtered and concentrated. The residue was purified by prep-HPLC using C18 reverse phase and eluting with 20-95% MeOH in H₂O to afford 5 mg of compound 94 as a white powder (yield=3%). LRMS (ESI): (calc) 375.1 (found) 376.2H+

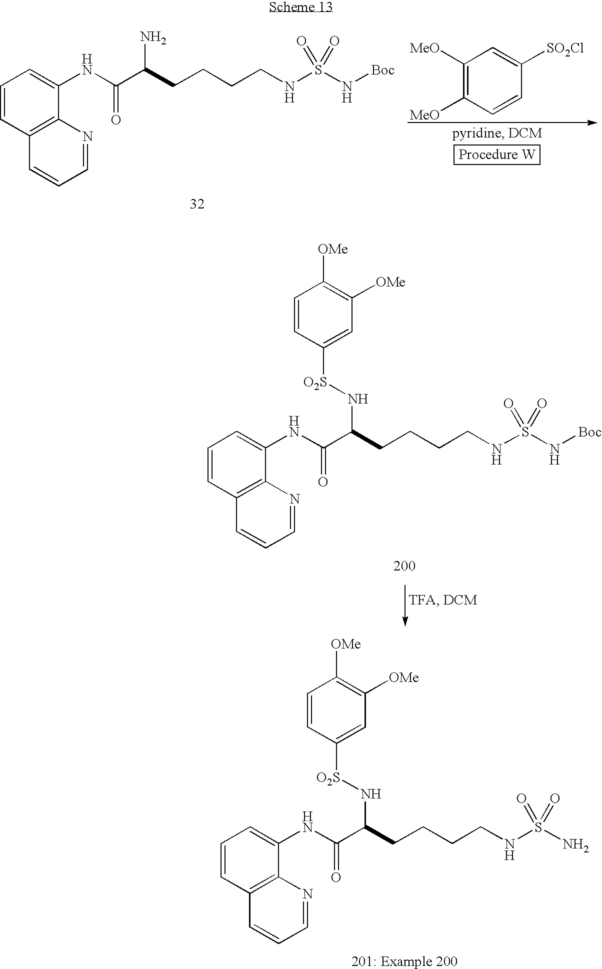

### Example 200

### (S)-2-(3,4-dimethoxyphenylsulfonamido)-N-(quinolin-8-yl)-6-(sulfamoylamino)hexanamide (201)

### Step 1: (S)-tert-butyl N-(5-(3,4-dimethoxyphenylsulfonamido)-6-oxo-6-(quinolin-8-ylamino)hexyl)sulfamoylcarbamate (200)

To a stirred solution of the amine 32 (127 mg, 0.28 mmol) in CH₂Cl₂ (3.5 mL) was added pyridine (0.03 mL) followed by 3,4-dimethoxybenzene-1-sulfonyl chloride (73 mg, 0.31 mmol). The solution was allowed to stir overnight and evaporated to dryness. The residue was purified by ISCO-40g with gradient of 30 to 100% EtOAC/hexanes. The title compound 200 (98 mg, 54%) was isolated as a white foam. LRMS: 651.8 (calc) 652.2 (found) (MH)+

### Step 2: (S)-2-(3,4-dimethoxyphenylsulfonamido)-N-(quinolin-8-yl)-6-(sulfamoylamino)hexanamide (201)

The general procedure B was used to convert carbamate 200 (98 mg, 0.15 mmol) to the crude sulfamide 201. Flash chromatography with 100% EtOAc gave 65 (46 mg, 56%) as a white solid. (DMSO-d6) δ (ppm): 10.58 (s, 1H), 8.89 (d, 1H, J=4.4 Hz), 8.51 (d, 1H, J=6.8 Hz), 8.41 (d, 1H, J=8.4 Hz), 8.35 (d, 1H, J=6.8 Hz), 7.66 (t, 1H, J=8.8 Hz), 7.63 (d, 1H, J=8.0 Hz), 7.54 (t, 1H, J=8.0 Hz), 7.37 (d, 1H, J=8.8 Hz), 7.28 (s, 1H), 6.92 (d, 1H, J=8.4 Hz), 6.42 (br, 2H), 6.36 (t, 1H, J=6.0 Hz), 3.88-3.86 (m, 1H), 3.69 (s, 3H), 3.66 (s, 3H), 2.70 (q, 2H, J=6.0 Hz), 1.66-1.54 (m, 2H), 1.32-1.11 (m, 4H). LRMS (ESI): (calc.) 551.2 (found) 552.4 (MH)+

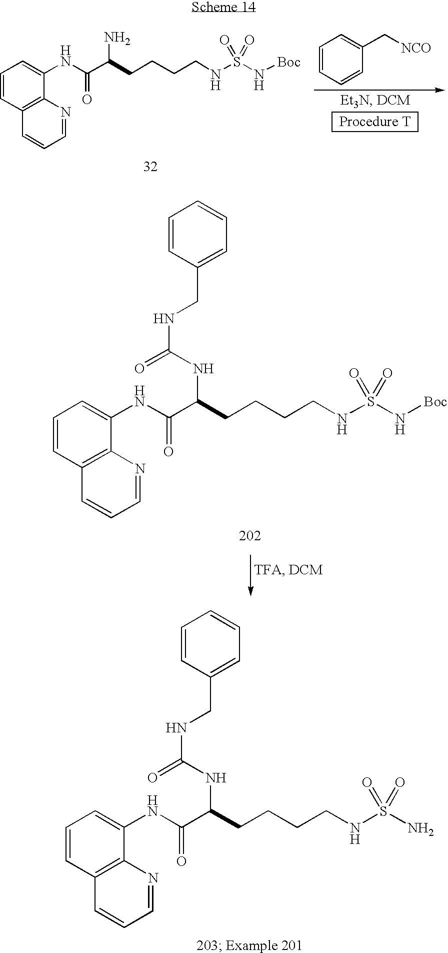

### Example 201

### (S)-2-(3-benzylureido)-N-(quinolin-8-yl)-6-(sulfamoylamino)hexanamide (203)

### Step 1: (S)-tert-butyl N-(5-(3-benzylureido)-6-oxo-6-(quinolin-8-ylamino)hexyl)sulfamoylcarbamate (202)

To a stirred solution of the amine 32 (127 mg, 0.28 mmol) in CH₂Cl₂ (3.5 mL) was added Et₃N (0.04 mL) followed by benzyl isocyanate (0.04 mL, 0.31 mmol). The solution was allowed to stir overnight and evaporated to dryness. The residue was purified by ISCO-40g with gradient of 30 to 100% EtOAC/hexanes. The title compound 202 (81 mg, 50%) was isolated as a white foam. LRMS: 584.7 (calc) 585.3 (found) (MH)+

### Step 2: (S)-2-(3-benzylureido)-N-(quinolin-8-yl)-6-(sulfamoylamino)hexanamide (203)

The general procedure B was used to convert carbamate 202 (81 mg, 0.14 mmol) to the sulfamide. Flash chromatography with 100% EtOAc gave the title compound 203 (38 mg, 56%) as a white solid. (CD₃OD) δ (ppm): 8.78 (d, 1H, J=4.4 Hz), 8.67 (d, 1H, J=7.6 Hz), 8.31 (d, 1H, J=8.0 Hz), 7.64 (d, 1H, J=7.6 Hz), 7.57-7.54 (m, 2H), 7.30 (d, 2H, J=6.4 Hz), 7.23-7.17 (m, 3H), 4.45 (d, 1H, J=15.2 Hz), 4.44 (q, 1H, J=4.8 Hz), 4.30 (d, 1H, J=15.6 Hz), 3.05 (t, 2H, J=7.2 Hz), 2.11-2.01 (m, 1H), 1.86-1.76 (m, 1H), 1.69-1.52 (m, 4H). LRMS (ESI): (calc.) 484.2 (found) 485.4 (MH)+

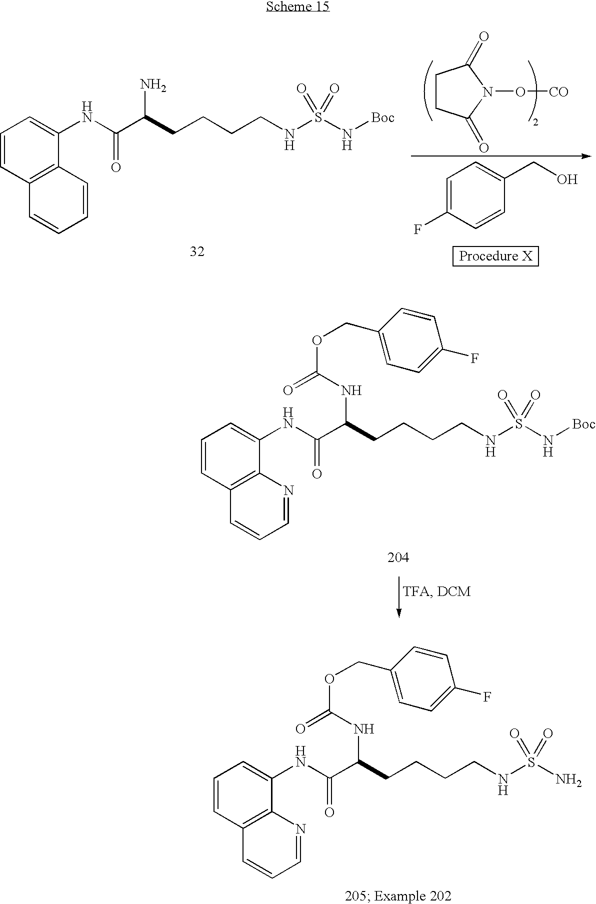

### Example 202

### (S)-4-fluorobenzyl 1-oxo-1-(quinolin-8-ylamino)-6-(sulfamoylamino)hexan-2-ylcarbamate (205)

### Step 1: (S)-4-fluorobenzyl 1-oxo-1-(quinolin-8-ylamino)-6-Boc-(sulfamoylamino)hexan-2-ylcarbamate (204)

p-F-BnOH (0.02 mL, 0.19 mmol) was dissolved in a 1:1 mixture of CH₂Cl₂-CH₃CN (10 mL) and iPr₂NEt (0.04 mL, 0.21 mmol) was added followed by disuccinimidyl carbonate (53 mg, 0.21 mmol) at 0° C. After stirring overnight, a solution of the amine 32 (85 mg, 0.19 mmol) in CH₂Cl₂ (0.5 mL) was added and the solution was stirred another hour at room temperature. The solution was evaporated and purified by ISCO-40g with gradient of 10 to 60% EtOAc/hexanes. The title compound 204 (43 mg, 38%) was isolated as a colorless oil. LRMS: 603.2 (calc) 604.4 (found) (MH)+

### Step 2: (S)-4-fluorobenzyl 1-oxo-1-(quinolin-8-ylamino)-6-(sulfamoylamino)hexan-2-ylcarbamate (205)

The general procedure B was used to convert carbamate 204 (43 mg, 0.07 mmol) to the crude sulfamide. Flash chromatography with 100% EtOAc gave the title compound 205 (22 mg, 62%) as a white solid. (CD₃OD) δ (ppm): 8.76 (dd, 1H, J=1.6, 4.0 Hz), 8.64 (d, 1H, J=7.2 Hz), 8.30 (dd, 1H, J=1.2, 8.0 Hz), 7.63 (d, 1H, J=8.0 Hz), 7.57-7.53 (m, 2H), 7.44-7.40 (m, 2H), 6.99 (t, 2H, J=8.4 Hz), 5.19 (d, 1H, J=12.4 Hz), 5.08 (d, 1H, J=12.4 Hz), 4.34 (q, 1H, J=4.8 Hz), 3.04 (t, 2H, J=6.8 Hz), 2.09-1.98 (m, 1H), 1.83-1.76 (m, 1H), 1.64-1.52 (m, 4H). LRMS (ESI): (calc.) 503.2 (found) 504.3 (MH)+.

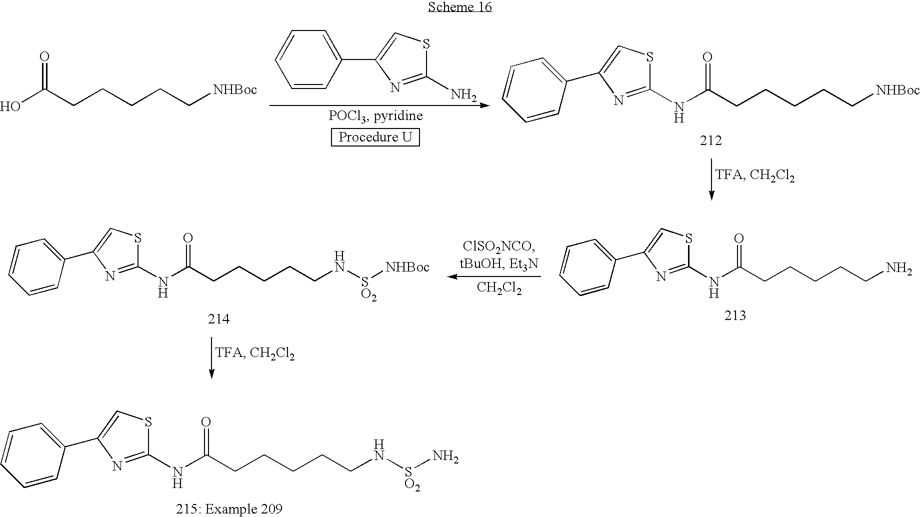

### Example 209

### N-(4-phenylthiazol-2-yl)-6-(sulfamoylamino)hexanamide (215)

### Step 1: tert-butyl 6-oxo-6-(4-phenylthiazol-2-ylamino)hexylcarbamate (212)

To a solution of 6-(tert-butoxycarbonylamino)hexanoic acid (463 mg, 2.0 mmol) in pyridine (8 ml) at 0° C. was added 2-amino-4-phenylthiazole hydrobromide monohydrate (660 mg, 2.4 mmol). Then phosphorous oxychloride (0.20 mL, 2.2 mmol) was added drop-wise. The mixture was stirred at 0° C. for 30 min, then at room temperature for 16 h. The mixture was quenched with water and extracted with ethyl acetate and the organic extract was washed with brine (X2), dried (MgSO₄), filtered, and evaporated. The residue was purified by silica gel column chromatography with ethyl acetate (40%) in hexanes to afford 212 (646 mg, 66%) as a white solid. LRMS (ESI): (calc.) 389.2; (found) 390.2 (MH)⁺.

### Step 2: 6-amino-N-(4-phenylthiazol-2-yl)hexanamide (213)

The general Procedure B was followed to afford 213 (340 mg, 71%) as a white solid. LRMS (ESI): (calc) 289.1; (found) 290.1 (MH)⁺.

### Step 3: tert-butyl N-(6-oxo-6-(4-phenylthiazol-2-ylamino)hexyl)sulfamoylcarbamate (214)

The general Procedure M was followed to afford 214 (211 mg, 38%) as a white solid. LRMS (ESI): (calc) 468.1; (found) 469.1 (MH)⁺.

### Step 4: N-(4-phenylthiazol-2-yl)-6-(sulfamoylamino)hexanamide (215)

The general Procedure B was followed to afford 215 (70 mg, 42%) as a white solid. (CD₃CN) δ (ppm) ¹H, 7.90 (dd, J=8.4, 1.4 Hz, 2H), 7.45 (t, J=7.2 Hz, 2H), 7.35 (t, J=7.4 Hz, 1H), 7.32 (s, 1H), 5.47 (bs, 2H), 5.00 (bt, 1H), 3.02 (q, J=6.8 Hz, 2H), 2.50 (t, J=7.2 Hz, 2H), 1.73 (qi, J=7.6 Hz, 2H), 1.59 (qi, J=7.2 Hz, 2H), 1.47-1.40 (m, 2H). LRMS (ESI): (calc) 368.1; (found) 369.2 (MH)⁺.

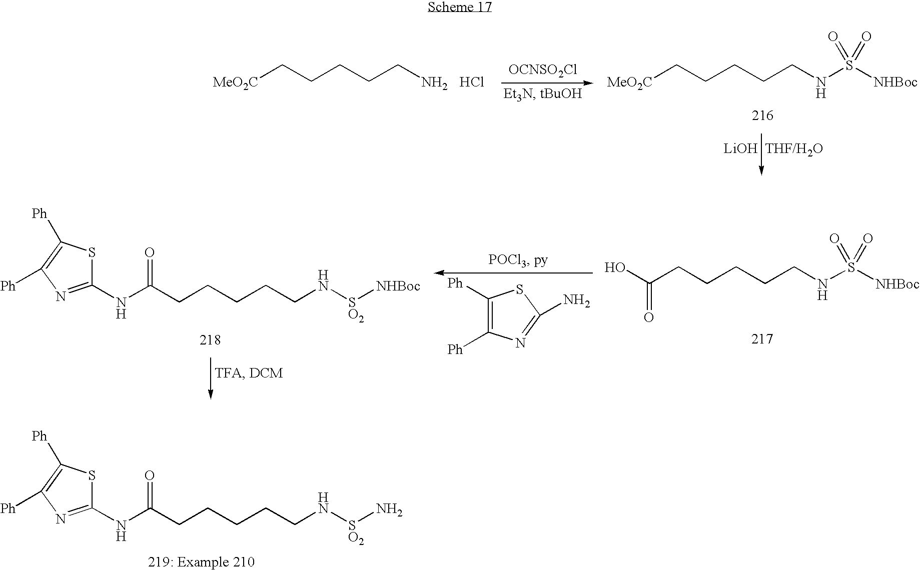

### Example 210

### N-(4,5-diphenylthiazol-2-yl)-6-(sulfamoylamino)hexanamide (219)

### Step 1: Methyl 6-(N-(tert-butoxycarbonyl)sulfamoylamino)hexanoate (216)

The general procedure M was used to prepare ester 216 (3.41 g, 96%) obtained as a white solid.

### Step 2: 6-(N-(tert-butoxycarbonyl)sulfamoylamino)hexanoic acid (217)

The general procedure J was used for the hydrolysis of ester 216 to give acid 217 (335 mg, 99%) as a white solid.

### Step 3: tert-butyl N-(6-(4,5-diphenylthiazol-2-ylamino)-6-oxohexyl)sulfamoylcarbamate (218)

The general procedure U was followed to afford 218 (25 mg, 57%) as a clear film. LRMS (ESI): (calc) 544.6; (found) 545.3 (MH)⁺.

### Step 4: N-(4,5-diphenylthiazol-2-yl)-6-(sulfamoylamino)hexanamide (219)

The general procedure B was followed to afford 219 (9 mg, 43%) as a white solid. (CD3CN) δ (ppm) 1H, 9.96 (bs, 1H), 7.47-7.43 (m, 2H), 7.37-7.34 (m, 5H), 7.33-7.29 (m, 3H), 5.14 (bs, 2H), 5.02 (bs, 1H), 3.02 (q, J=7.2 Hz, 2H), 2.50 (t, J=7.2 Hz, 1H), 1.79-1.69 (m, 2H), 1.62 (m, 2H), 1.48-1.41 (m, 2H). LRMS (ESI): (calc) 444.5; (found) 445.2 (MH)⁺.

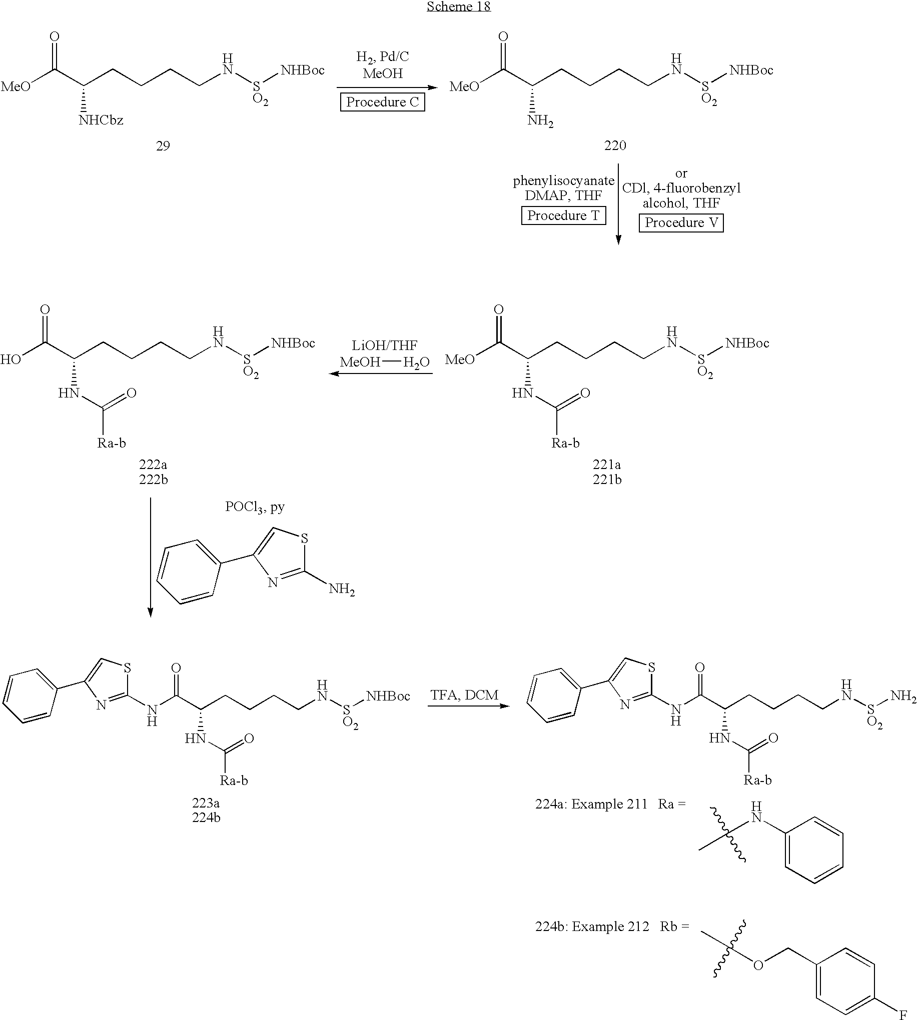

### Example 211

### (S)—N-(4-phenylthiazol-2-yl)-2-(3-phenylureido)-6-(sulfamoylamino)hexanamide (224a)

### Step 1: (S)-methyl 2-amino-6-(N-(tert-butoxycarbonyl)sulfamoylamino)hexanoate (220)

The general procedure C was followed to afford 220 (2.60 g, quant.) as a white solid. LRMS (ESI): (calc) 339.4; (found) 340.2 (MH)⁺.

### Step 2: (S)-methyl 6-(N-(tert-butoxycarbonyl)sulfamoylamino)-2-(3-phenylureido)hexanoate (221a)

The general procedure T was followed to afford 221a (405 mg, quant.) as a white solid. LRMS (ESI): (calc) 458.4; (found) 459.2 (MH)⁺.

### Step 3: (S)-6-(N-(tert-butoxycarbonyl)sulfamoylamino)-2-(3-phenylureido)hexanoic acid (222a)

The general procedure J was followed to afford 222a (404 mg, quant.) as a white solid. LRMS (ESI): (calc) 444.5; (found) 443.1 (MH)⁻.

### Step 4: (S)-tert-butyl N-(6-oxo-6-(4-phenylthiazol-2-ylamino)-5-(3-phenylureido)hexyl)sulfamoylcarbamate (223a)

The general procedure U was followed to afford 223a (20 mg, 4% over 3 steps) as a white solid.

LRMS (ESI): (calc) 602.7; (found) 603.2 (MH)⁺.

### Step 5: (S)—N-(4-phenylthiazol-2-yl)-2-(3-phenylureido)-6-(sulfamoylamino)hexanamide (224a)

The general procedure B was followed to afford 224a (16 mg, 96%) as a white solid. (CD₃CN) δ (ppm) 1H, 7.90-7.88 (m, 2H), 7.50 (bs, 1H), 7.45-7.41 (m, 4H), 7.36-7.27 (m, 4H), 7.03-6.99 (m, 1H), 5.81 (d, J=7.2 Hz, 1H), 5.16-5.07 (m, 3H), 4.51-4.45 (m, 1H), 3.04-3.00 (m, 2H), 1.98-1.90 (m, 1H), 1.82-1.74 (m, 1H), 1.65-1.46 (m, 4H). LRMS (ESI): (calc) 502.6; (found) 503.2 (MH)⁺.

### Example 212

### (S)-4-fluorobenzyl 1-oxo-1-(4-phenylthiazol-2-ylamino)-6-(sulfamoylamino)hexan-2-ylcarbamate (224b)

### Step 1: (S)-methyl 6-(N-(tert-butoxycarbonyl)sulfamoylamino)-2-((4-fluorobenzyloxy)carbonylamino)hexanoate (221b)

4-fluorobenzyl alcohol (96 mg, 0.88 mmol) was added to a suspension of CDI (143 mg, 0.88 mmol) in DCM (2 mL) at 0° C. The resulting solution was warmed to room temperature and stirred for 1 hour. Amine 220 (300 mg, 0.88 mmol) was added and the reaction stirred overnight. Water was added and the aqueous phase was extracted with EtOAc and the organic extract was dried (Na₂SO₄), filtered and evaporated. The residue was purified by silica gel chromatography with gradient of EtOAc (20-40%) in Hexane to afford 221b (117 mg, 27%). LRMS (ESI): (calc) 491.5; (found) 514.2 (M+Na)⁺.

### Step 2: (S)-6-(N-(tert-butoxycarbonyl)sulfamoylamino)-2-((4-fluorobenzyloxy)carbonylamino)hexanoic acid (222b)

The general procedure J was followed to afford 222b (51 mg, 45%) as a white solid. LRMS (ESI): (calc) 477.5; (found) 500.2 (MH)⁺.

### Step 3: (S)-6-(N-(tert-butoxycarbonyl)sulfamoylamino)-2-((4-fluorobenzyloxy)carbonylamino)hexanoic acid N-(4-phenylthiazol-2-yl)amide (223b)

The general procedure U was followed to afford 223b (31 mg, 32%) as a white solid. LRMS (ESI): (calc) 602.7; (found) 603.2 (MH)⁺.

### Step 4: (S)-4-fluorobenzyl 1-oxo-1-(4-phenylthiazol-2-ylamino)-6-(sulfamoylamino)hexan-2-ylcarbamate (224b)

The general procedure B was followed to afford 224b (23 mg, 88%) as a white solid. (CD₃CN) δ (ppm) 1H, 10.23 (bs, 1H), 7.91-7.88 (m, 2H), 7.46-7.40 (m, 4H), 7.37-7.33 (m, 2H), 7.14-7.10 (m, 2H), 6.1 (d, J=7.6 Hz, 1H), 5.16-5.03 (m, 5H), 4.35-4.32 (m, 1H), 3.00 (q, J=6.8 Hz, 2H), 1.90-1.86 (m, 1H), 1.76-1.71 (m, 1H), 1.60-1.43 (m, 4H). LRMS (ESI): (calc) 535.6; (found) 536.2 (MH)⁺.

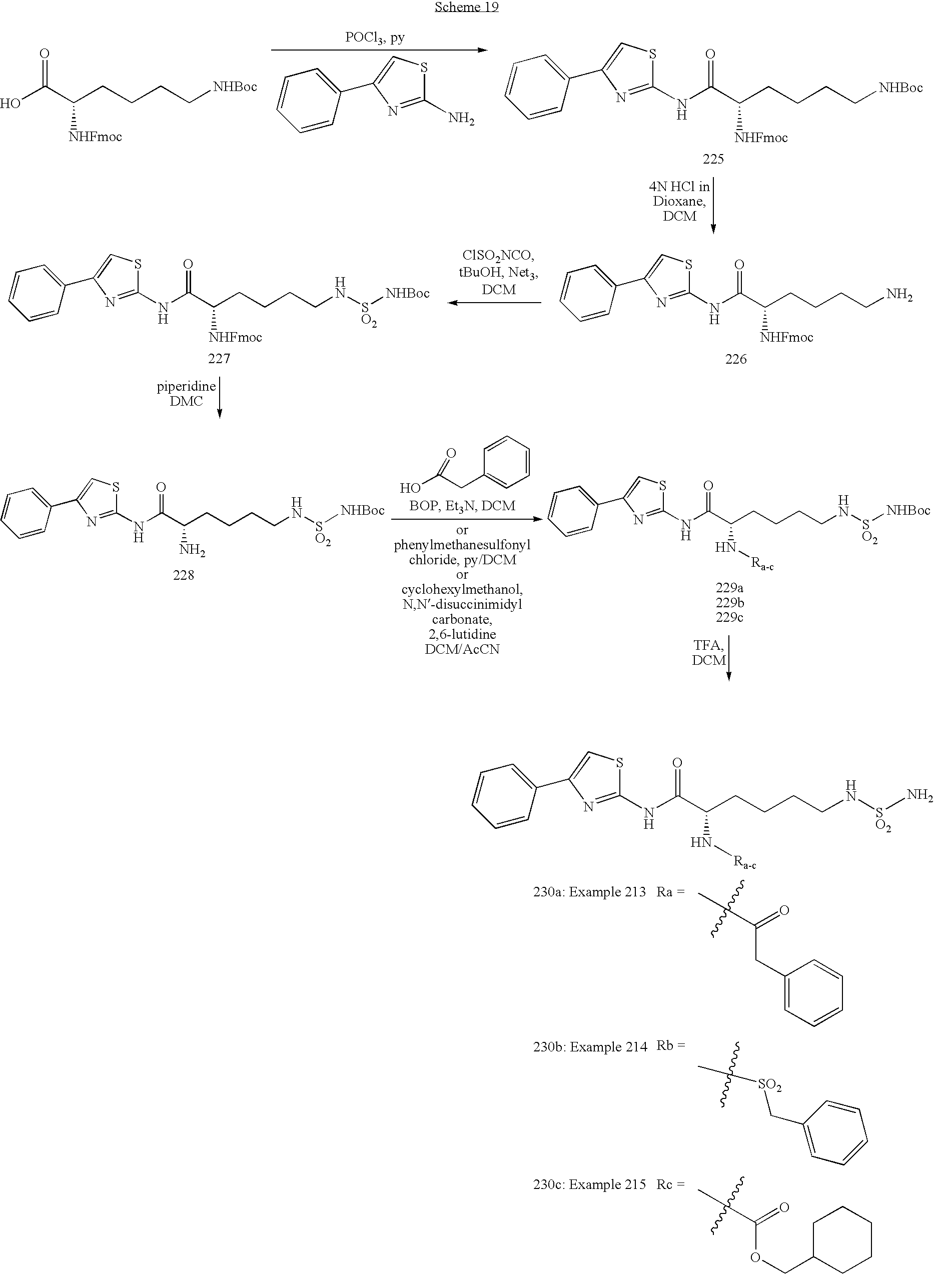

### Example 213

### (S)-2-(2-phenylacetamido)-N-(4-phenylthiazol-2-yl)-6-(sulfamoylamino)hexanamide (230a)

### Step 1: (S)-2-(((9H-fluoren-9-yl)methoxy)carbonylamino)-6-(tert-butoxycarbonylamino)hexanoic acid N-(4-phenylthiazol-2-yl)amide (225)

The general procedure U was followed to afford 225 (4.36 g, 65%) as a yellow foam. LRMS (ESI): (calc) 626.7; (found) 627.4 (MH)⁺.

### Step 2: (S)-(9H-fluoren-9-yl)methyl 6-amino-1-oxo-1-(4-phenylthiazol-2-ylamino)hexan-2-ylcarbamate (226)

The general procedure E was followed to afford 226 (895 mg, 100% crude).

LRMS (ESI): (calc) 526.6; (found) 527.3 (MH)⁺.

### Step 3: (S)-2-(((9H-fluoren-9-yl)methoxy)carbonylamino)-6-(N-(tert-butoxycarbonyl)sulfamoylamino)hexanoic acid N-(4-phenylthiazol-2-yl)amide (227)

The general procedure M was followed to afford 227 (3.95 g, 75%) as a yellow solid. LRMS (ESI): (calc) 705.8; (found) 706.2 (MH)⁺.

### Step 4: (S)-tert-butyl N-(5-amino-6-oxo-6-(4-phenylthiazol-2-ylamino)hexyl)sulfamoylcarbamate (228)

To a solution of 227 (720 mg, 1.02 mmol) in dichloromethane (4 ml) was added piperidine (1.0 mL). The mixture was stirred at room temperature for 15 min. The mixture was concentrated and the residue was purified by silica gel column chromatography with methanol (10%) in dichloromethane to afford 228 (426 mg, 69%) as a yellow solid.

LRMS (ESI): (calc) 483.6; (found) 484.2 (MH)⁺.

### Step 5: (S)-2-(2-phenylacetamido)-N-(4-phenylthiazol-2-yl)-6-(sulfamoylamino)hexanamide (230a)

The general procedure A was used to obtain compound 229a followed by procedure B to afford 230a (16 mg, 13%) as a white solid. (CD₃CN) δ (ppm) 1H: 7.91-7.88 (m, 2H), 7.47-7.42 (m, 2H), 7.38-7.34 (m, 5H), 7.33-7.00 (m, 1H), 6.99 (d, J=6.8 Hz, 1H), 5.13 (s, 2H), 5.00 (b-triplet, 1H), 4.54-4.48 (m, 1H), 3.60 (s, 2H), 2.99 (quartet, J=6.8 Hz, 2H), 1.92-1.87 (m, 1H), 1.79-1.70 (m, 1H), 1.60-1.52 (m, 2H), 1.48-1.39 (m, 2H). LRMS (ESI): (calc) 501.6; (found) 502.0 (MH)⁺.

### Example 214

### (S)-2-(phenylmethylsulfonamido)-N-(4-phenylthiazol-2-yl)-6-(sulfamoylamino)hexanamide (230b)

### Step 1: (S)-tert-butyl N-(6-oxo-5-(phenylmethylsulfonamido)-6-(4-phenylthiazol-2-ylamino)hexyl)sulfamoylcarbamate (229b)

The general procedure W was used to afford compound 229b. Silica gel chromatography with gradient of EtOAc (20-40%) in Hexane gave 229b (23 mg, 23%) as a white solid.

LRMS (ESI): (calc) 637.7; (found) 638.3 (MH)⁺.

### Step 2: (S)-2-(phenylmethylsulfonamido)-N-(4-phenylthiazol-2-yl)-6-(sulfamoylamino)hexanamide (230b)

The general procedure B was followed to afford 230b (4 mg, 21%) as a white solid. (CD3CN) δ (ppm) 1H: 7.91-7.88 (m, 2H), 7.55-7.29 (m, 9H), 5.95 (b-doublet, 1H), 5.13-4.95 (m, 3H), 4.39 (s, 2H), 1.07-4.00 (m, 1H), 3.04-2.97 (m, 2H), 1.96-1.65, 2H), 1.64-1.38 (m, 4H). LRMS (ESI): (calc) 537.6; (found) 538.2 (MH)⁺.

### Example 215

### (S)-cyclohexylmethyl 1-oxo-1-(4-phenylthiazol-2-ylamino)-6-(sulfamoylamino)hexan-2-ylcarbamate (230c)

### Step 1: (S)-6-(N-(tert-butoxycarbonyl)sulfamoylamino)-2-((cyclohexylmethoxy)carbonylamino)hexanoic acid N-(4-phenylthiazol-2-yl)amide (229c)

The general procedures X was followed to afford 229c. LRMS (ESI): (calc) 623.7; (found) 624.4 (MH)⁺.

### Step 2: (S)-cyclohexylmethyl 1-oxo-1-(4-phenylthiazol-2-ylamino)-6-(sulfamoylamino)hexan-2-ylcarbamate (230c)

The general procedure B was followed to afford 230c (13 mg, 60%) as a white solid. (CD₃CN) δ (ppm) 1H: 7.91-7.89 (m, 2H), 7.46-7.42 (m, 2H), 7.37-7.35 (m, 2H), 6.05 (bs, 1H), 5.16 (bs, 2H), 5.05 (bs, 1H), 4.35-4.28 (m, 1H), 3.88 (d, J=6.0 Hz, 2H), 3.00 (q, J=6.4 Hz, 2H), 1.93-1.88 (m, 1H), 1.75-1.41 (m, 11H), 1.32-1.17 (m, 3H), 1.03-0.98 (m, 2H). LRMS (ESI): (calc) 523.6; (found) 524.3 (MH)⁺.

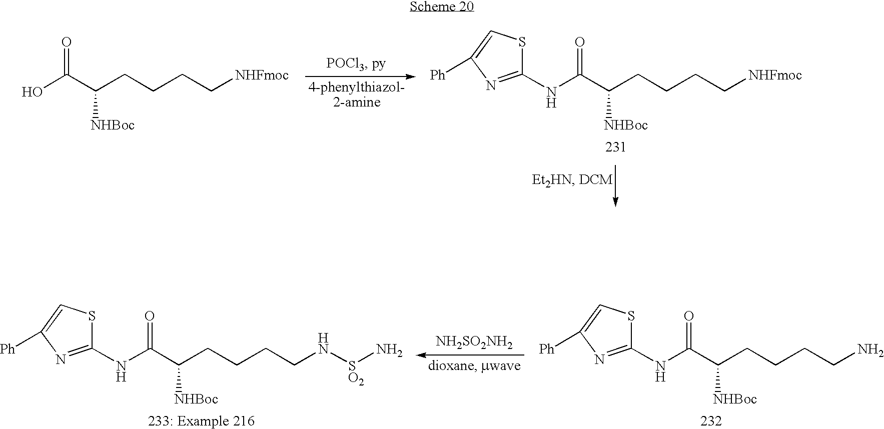

### Example 216

### (S)-tert-butyl 1-oxo-1-(4-phenylthiazol-2-ylamino)-6-(sulfamoylamino)hexan-2-ylcarbamate (233)

### Step 1: (S)-6-(((9H-fluoren-9-yl)methoxy)carbonylamino)-2-(tert-butoxycarbonylamino)hexanoic acid N-(4-phenylthiazol-2-yl)amide (231)

The general procedure U was followed to afford 231 (41 mg, 10%) as a clear film. LRMS (ESI): (calc) 626.7; (found) 627.4 (MH)⁺.

### Steps 2-3: (S)-tert-butyl 1-oxo-1-(4-phenylthiazol-2-ylamino)-6-(sulfamoylamino)hexan-2-ylcarbamate (233)

Diethylamine (2 mL) was added to a solution of 231 (81 mg, 0.13 mmol) in DCM (0.5 mL) and the mixture was stirred for 4 h at room temperature. The solvent was evaporated and crude 232 was dissolved in dioxane (0.50 mL) and added to a pressure tube containing sulfamide (118 mg, 1.23 mmol) in dioxane (0.50 mL). The mixture was placed in the microwave for 4 min. The reaction was cooled to room temperature, water was added and the aqueous phase was extracted with EtOAc. The organic extract was dried (Na₂SO₄), filtered and evaporated. The residue was purified by silica gel chromatography with gradient of EtOAc (40-60%) in Hexane to afford 233 (4 mg, 7%) as a white solid.

(CD₃CN) δ (ppm) 1H: 7.91-7.88 (m, 2H), 7.47-7.42 (m, 2H), 7.42-7.35 (m, 2H), 5.81 (d, J=5.6 Hz, 1H), 5.15 (bs, 2H), 5.03 (bm, 1H), 4.26 (bs, 2H), 3.01 (q, J=6.4 Hz, 2H), 1.95-1.81 (m, 1H), 1.73-1.68 (m, 1H), 1.62-1.56 (m, 2H), 1.54-1.44 (m, 11H). LRMS (ESI): (calc) 483.6; (found) 484.2 (MH)⁺.

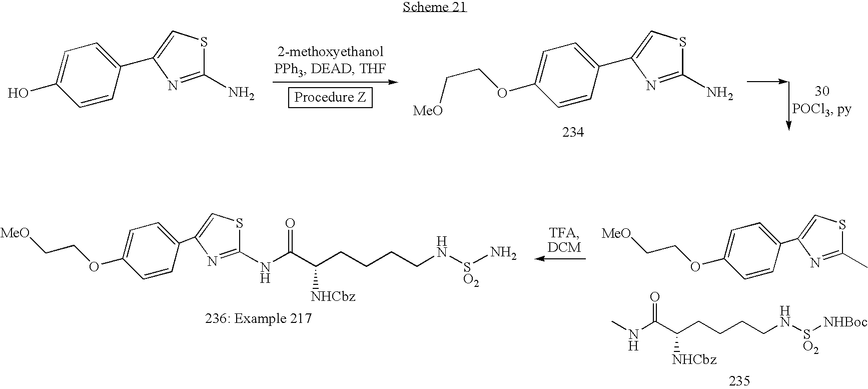

### Example 217

### (S)-benzyl 1-(4-(4-(2-methoxyethoxy)phenyl)thiazol-2-ylamino)-1-oxo-6-(sulfamoylamino)hexan-2-ylcarbamate (236)

### Step 1: 4-(4-(2-methoxyethoxy)phenyl)thiazol-2-amine (234)

To a solution 4-(2-aminothiazol-4-yl)phenol (200 mg, 1.04 mmol) and 2-methoxyethanol (0.082 mL, 1.04 mmol) in THF (7 mL) was added triphenylphosphine (409 mg, 1.56 mmol) and DEAD (0.19 mL, 1.24 mmol) sequentially. The reaction stirred at room temperature overnight. The solvent was evaporated and the residue was purified by silica gel chromatography with gradient of EtOAc (20-40%) in Hexane to afford 234 (67 mg, 26%) as a white solid.

LRMS (ESI): (calc) 250.3; (found) 251.2 (MH)⁺.

### Step 2-3: (S)-benzyl 1-(4-(4-(2-methoxyethoxy)phenyl)thiazol-2-ylamino)-1-oxo-6-(sulfamoylamino)hexan-2-ylcarbamate (236)

The general procedures U and B were followed in the order mentioned to afford 236 (38 mg, 15%) as a white solid.

(CD₃CN) δ (ppm) 1H, 7.82 (d, J=8.8 Hz, 2H), 7.40-7.36 (m, 5H), 7.21 (s, 1H), 6.98 (d, J=8.8 Hz, 2H), 6.17 (d, J=6.8 Hz, 1H), 5.14-5.03 (m, 5H), 4.33-4.30 (m, 1H), 4.16-4.13 (m, 2H), 3.73-3.71 (m, 2H), 3.39 (s, 3H), 3.00 (q, J=6.4 Hz, 2H), 1.94-1.89 (m, 1H), 1.76-1.72 (m, 1H), 1.58-1.43 (m, 4H). LRMS (ESI): (calc) 591.7; (found) 592.3 (MH)⁺.

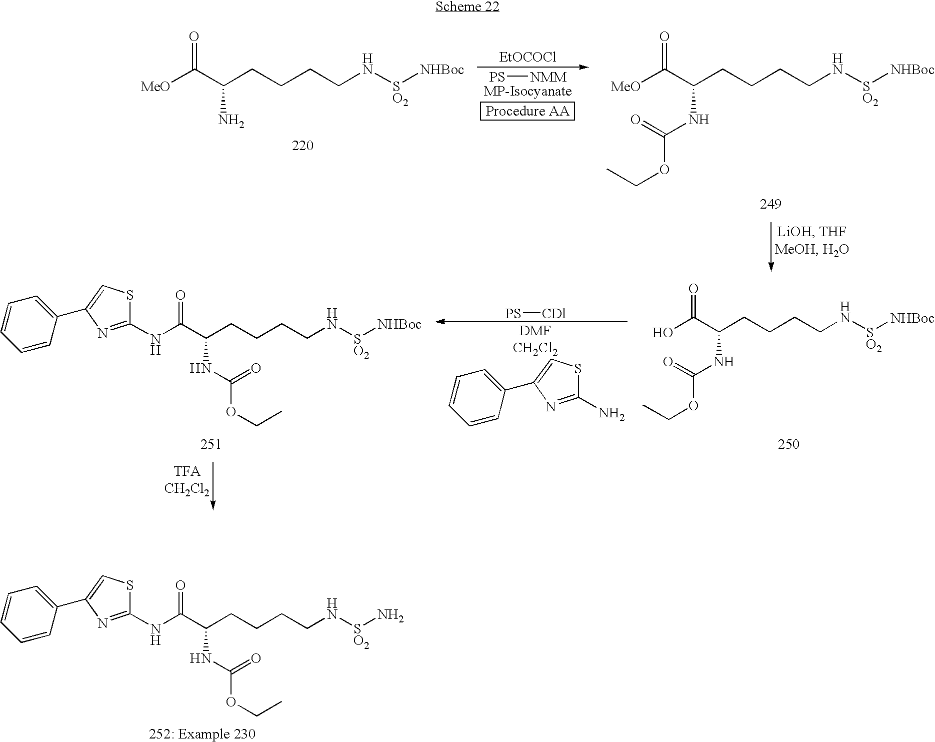

### Example 230

### (S)-ethyl 1-oxo-1-(4-phenylthiazol-2-ylamino)-6-(sulfamoylamino)hexan-2-ylcarbamate (252)

### Step 1: (S)-methyl 6-(N-(tert-butoxycarbonyl)sulfamoylamino)-2-(ethoxycarbonylamino)hexanoate (249)

To a solution of PS-NMM (877 mg, 2.0 mmol) in dichloromethane (6 mL) was added 220 (407 mg, 1.2 mmol). The mixture was cooled to 0° C., and then ethyl chloroformate (96 uL, 1.0 mmol) was added dropwise. The mixture was stirred at room temperature for 16 h. Then excess 220 was scavenged with MP-Isocyanate (1.39 g, 2.0 mmol). After 3h at room temperature the mixture was filtered and the solvent was evaporated to afford 249 (164 mg, 40%) as a white solid. LRMS (ESI): (calc.) 411.2; (found) 434.2 (M+Na)⁺.

### Step 2: (S)-6-(N-(tert-butoxycarbonyl)sulfamoylamino)-2-(ethoxycarbonylamino)hexanoic acid (250)

The general Procedure J was followed to afford 250 (151 mg, 95%) as a white solid. LRMS (ESI): (calc) 397.2; (found) 404.2 (M+Li)⁺.

### Step 3: (S)-6-(N-(tert-butoxycarbonyl)sulfamoylamino)-2-(ethoxycarbonylamino)hexanoic acid N-(4-phenylthiazol-2-yl)amide (251)

The general Procedure D was followed to afford 251 that was used crude for next step. LRMS (ESI): (calc) 555.2; (found) 556.3 (MH)⁺.

### Step 4: (S)-ethyl 1-oxo-1-(4-phenylthiazol-2-ylamino)-6-(sulfamoylamino)hexan-2-ylcarbamate (252)

The general Procedure B was followed to afford 252 (20 mg, 13%) as a white solid. (DMSO-d₆) δ (ppm) ¹H, 12.36 (s, 1H), 7.88 (d, J=7.0 Hz, 2H), 7.62 (s, 1H), 7.49 (d, J=7.2 Hz, 1H), 7.42 (t, J=7.2 Hz, 2H), 7.31 (t, J=7.2 Hz, 1H), 6.44 (s, 2H), 6.43 (t, J=6.5 Hz, 1H), 4.22 (q, J=5.1 Hz, 1H), 3.97 (q, J=7.0 Hz, 2H), 2.83 (q, J=6.3 Hz, 2H), 1.77-1.23 (m, 6H), 1.16 (t, J=7.0 Hz, 3H). LRMS (ESI): (calc) 455.1; (found) 456.2 (MH)⁺.

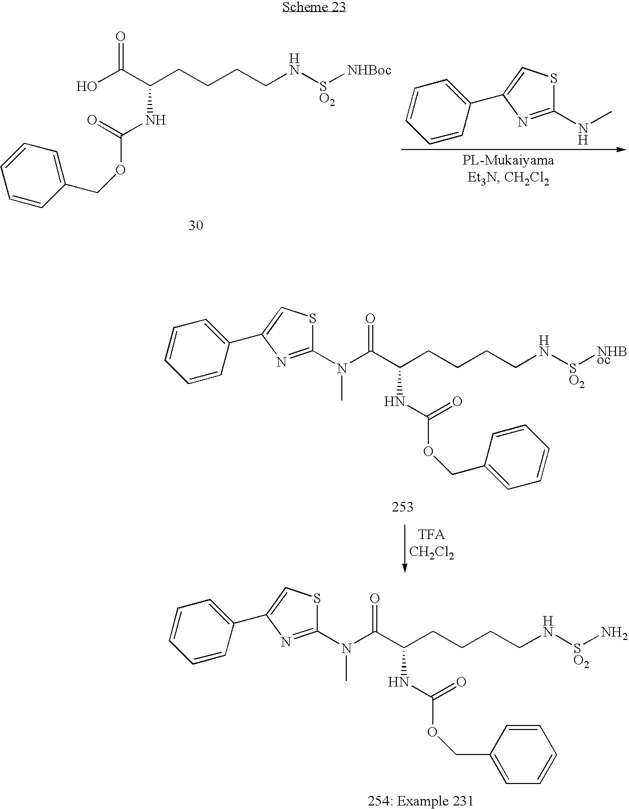

### Example 231

### (S)-benzyl 1-(methyl(4-phenylthiazol-2-yl)amino)-1-oxo-6-(sulfamoylamino)hexan-2-ylcarbamate (254)

### Step 1: (S)-6-(N-(tert-butoxycarbonyl)sulfamoylamino)-2-((benzyloxy)carbonylamino)hexanoic acid N-methyl-(4-phenylthiazol-2-yl)amide (253)

To a solution of 30 (92 mg, 0.20 mmol) in dichloromethane (3 mL) was added N-methyl-4-phenylthiazol-2-amine (38 mg, 0.20 mmol), triethylamine (84 uL, 0.60 mmol) and PL-Mukaiyama (378 mg, 0.40 mmol). The mixture was stirred at room temperature for 16 h. Then the mixture was filtered and a saturated solution of ammonium chloride added, followed by ethyl acetate extractions. The organic extract was dried (MgSO₄), filtered, and evaporated. The crude product was purified using SCX-2 cartridge (1 g) with dichloromethane to afford 253 (49 mg, 39%). LRMS (ESI): (calc.) 631.2; (found) 632.3 (MH)⁺.

### Step 2: (S)-benzyl 1-(methyl(4-phenylthiazol-2-yl)amino)-1-oxo-6-(sulfamoylamino)hexan-2-ylcarbamate (254)

The general Procedure B was followed to afford 254 (17 mg, 22%) as a white solid. (DMSO-d6) δ (ppm) ¹H, 7.95-7.90 (m, 3H), 7.70 (s, 1H), 7.42 (t, J=7.4 Hz, 2H), 7.38-7.28 (m, 5H), 7.12 (s, 1H), 6.45-6.42 (m, 3H), 5.03 (s, 2H), 4.73-4.67 (m, 1H), 3.82 (s, 3H), 2.84 (q, J=6.3 Hz, 2H), 1.80-1.55 (m, 2H), 1.55-1.32 (m, 4H). LRMS (ESI): (calc) 531.2; (found) 532.3 (MH)⁺.

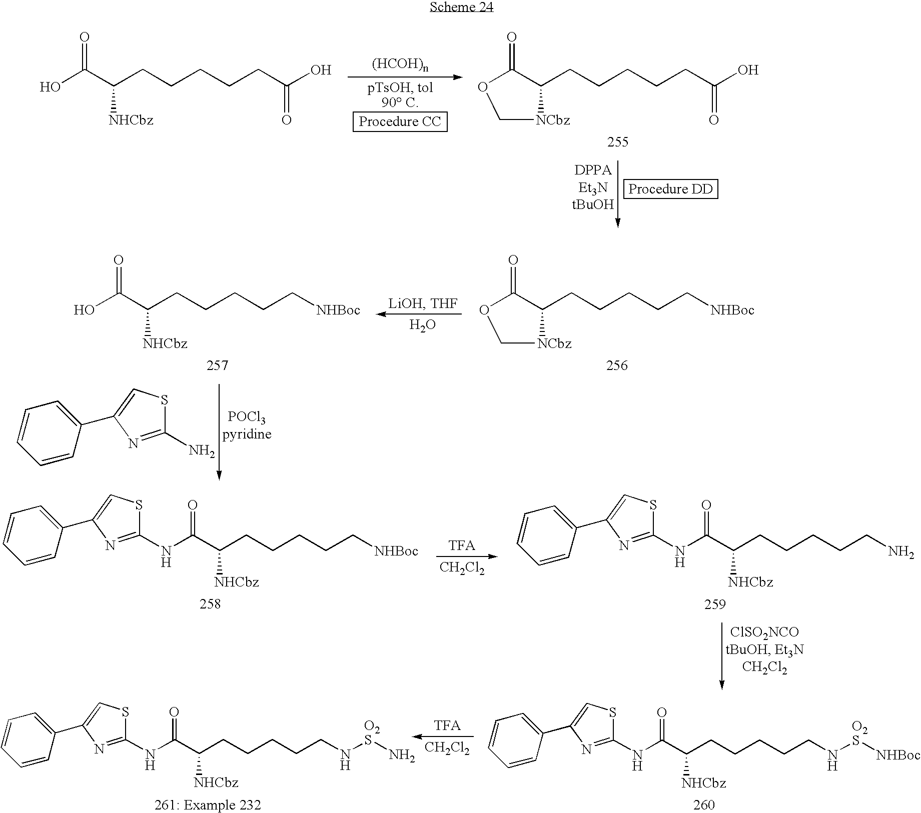

### Example 232

### (S)-benzyl 1-oxo-1-(4-phenylthiazol-2-ylamino)-7-(sulfamoylamino)heptan-2-ylcarbamate (261)

### Step 1: (S)-6-(3-(benzyloxycarbonyl)-5-oxooxazolidin-4-yl)hexanoic acid (255)

To a solution of (S)-2-(benzyloxycarbonylamino)octanedioic acid (1.49 g, 4.62 mmol) in toluene (12 ml) was added paraformaldehyde (149 mg) and p-toluenesulfonic acid monohydrate (88 mg, 0.46 mmol). The mixture was stirred for 30 min at 90° C. After cooling, the solvent was evaporated, water added and the mixture extracted with ethyl acetate. The organic extract was dried (MgSO₄), filtered, and evaporated. The residue was purified by silica gel column chromatography with ethyl acetate (40%) in hexanes to afford 255 (625 mg, 40%) as a colorless oil. LRMS (ESI): (calc.) 335.1; (found) 336.2 (MH)⁺.

### Step 2: (S)-benzyl 4-(5-(tert-butoxycarbonylamino)pentyl)-5-oxooxazolidine-3-carboxylate (256)

To a solution of 75 (625 mg, 1.87 mmol) in tBuOH (8 ml) was added triethylamine (0.26 mL, 1.87 mmol)) and diphenylphosphoryl azide (0.44 mL, 2.05 mmol). The mixture was stirred at for 16 h at 80° C. After cooling, the mixture was quenched with a saturated solution of ammonium chloride, and extracted with ethyl acetate. The organic extract was dried (MgSO₄), filtered, and evaporated. The residue was purified by silica gel column chromatography with ethyl acetate (40%) in hexanes to afford 256 (232 mg, 31%) as a colorless oil. LRMS (ESI): (calc.) 406.2; (found) 429.3 (M+Na)⁺.

### Step 3: (S)-2-(benzyloxycarbonylamino)-7-(tert-butoxycarbonylamino)heptanoic acid (257)

The general Procedure J was followed to afford 257 (212 mg, 94%) as a white solid. LRMS (ESI): (calc) 394.2; (found) 417.3 (M+Na)⁺.

### Step 4: (S)-2-(benzyloxycarbonylamino)-7-(tert-butoxycarbonylamino)heptanoic acid N-(4-phenylthiazol-2-yl)amide (258)

The general Procedure U was followed to afford 258 (282 mg, 90%) as a white solid. LRMS (ESI): (calc) 552.2; (found) 553.3 (MH)⁺.

### Step 5: (S)-benzyl 7-amino-1-oxo-1-(4-phenylthiazol-2-ylamino)heptan-2-ylcarbamate (259)

The general Procedure B was followed to afford 259 (220 mg, 95%) as a white solid. LRMS (ESI): (calc) 452.2; (found) 453.3 (MH)⁺.

### Step 6: (S)-2-(benzyloxycarbonylamino)-7-(N-(tert-butoxycarbonyl)sulfamoylamino)heptanoic acid N-(4-phenylthiazol-2-yl)amide (260)

The general Procedure M was followed to afford 260 (165 mg, 54%) as a white solid. LRMS (ESI): (calc) 631.2; (found) 632.3 (MH)⁺.

### Step 7: (S)-benzyl 1-oxo-1-(4-phenylthiazol-2-ylamino)-7-(sulfamoylamino)heptan-2-ylcarbamate (261)

The general Procedure B was followed to afford 261 (86 mg, 62%) as a white solid. (DMSO-d6) δ (ppm) ¹H, 12.39 (s, 1H), 7.89 (d, J=7.2 Hz, 2H), 7.71 (d, J=7.2 Hz, 1H), 7.63 (s, 1H), 7.42 (t, J=7.4 Hz, 2H), 7.36-7.10 (m, 6H), 6.42 (m, 3H), 5.021 (d, J=2.9 Hz, 2H), 4.29-4.25 (m, 1H), 2.82 (,t J=7.0 Hz, 2H), 1.76-1.57 (m, 2H), 1.51-1.21 (m, 6H). LRMS (ESI): (calc) 531.1; (found) 532.1 (MH)⁺.

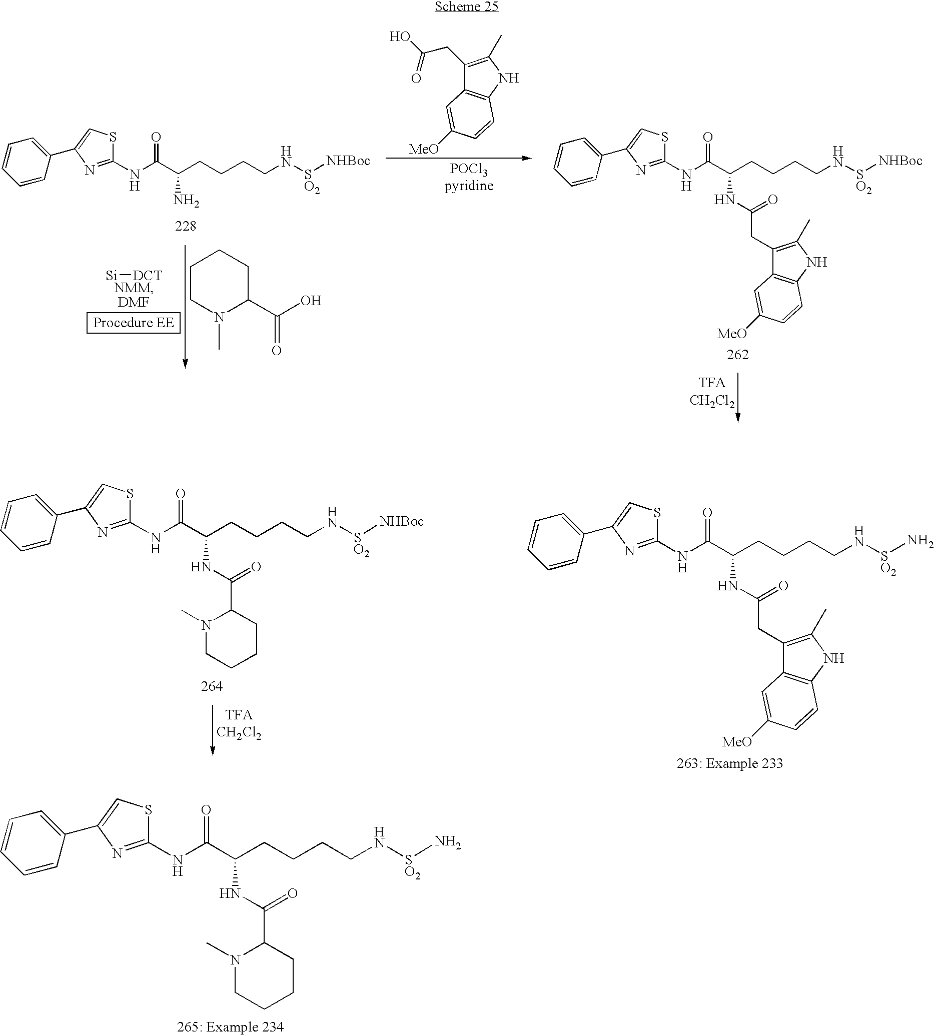

### Example 233

### (S)-2-(2-(5-methoxy-2-methyl-1H-indol-3-yl)acetamido)-N-(4-phenylthiazol-2-yl)-6-(sulfamoylamino)hexanamide (263)

### Step 1: (S)-tert-butyl N-(5-(2-(5-methoxy-2-methyl-1H-indol-3-yl)acetamido)-6-oxo-6-(4-phenylthiazol-2-ylamino)hexyl)sulfamoylcarbamate (262)

The general Procedure U was followed to afford 262 (117 mg, 34%) as a white solid. LRMS (ESI): (calc) 684.2; (found) 685.2 (MH)⁺.

### Step 2: (S)-2-(2-(5-methoxy-2-methyl-1H-indol-3-yl)acetamido)-N-(4-phenylthiazol-2-yl)-6-(sulfamoylamino)hexanamide (263)

The general Procedure B was followed to afford 263 (37 mg, 37%) as a white solid. (DMSO-d6) δ (ppm) ¹H, 12.36 (s, 1H), 10.57 (s, 1H), 8.30 (d, J=7.4 Hz, 1H), 7.87 (dd, J=8.4, 1.4 Hz, 2H), 7.61 (s, 1H), 7.41 (t, J=7.4 Hz, 2H), 7.30 (tt, J=7.2, 1.2 Hz, 1H), 7.07 (d, J=8.6 Hz, 1H), 7.01 (d, J=2.3 Hz, 1H), 6.58 (dd, J=8.6, 2.3 Hz, 1H), 6.43 (m, 3H), 4.45 (q, J=8.4 Hz, 1H), 3.72 (s, 3H), 3.48 (q, J=13 Hz, 2h), 2.84-2.78 (m, 2H), 2.30 (s, 3H), 1.81-1.60 (M, 2H), 1.49-1.22 (m, 4H). LRMS (ESI): (calc) 584.2; (found) 585.2 (MH)⁺.

### Example 234

### 1-methyl-N—((S)-1-oxo-1-(4-phenylthiazol-2-ylamino)-6-(sulfamoylamino)hexan-2-yl)piperidine-2-carboxamide (265)

### Step 1: tert-butyl N-((5S)-5-(1-methylpiperidine-2-carboxamido)-6-oxo-6-(4-phenylthiazol-2-ylamino)hexyl)sulfamoylcarbamate (264)

To a solution of 1-methylpiperidine-2-carboxylic acid (107 mg, 0.75 mmol) in DMF (3.3 ml) was added Si-DCT (1.7 g, 1.0 mmol, Silica-bond dichlorotriazine), 228 (242 mg, 0.5 mmol) and N-methylmorpholine (0.16 mL, 1.5 mmol). The mixture was stirred at room temperature for 16 h. The mixture was filtrated, then concentrated. The residue was diluted with dichloromethane and washed with brine, and then dried (MgSO₄), filtered, and evaporated. The residue was purified by silica gel column chromatography with methanol (10%) in dichloromethane to afford 264 (232 mg, 76%) as a light yellow solid. LRMS (ESI): (calc.) 608.2; (found) 609.2 (MH)⁺.

### Step 2: 1-methyl-N—((S)-1-oxo-1-(4-phenylthiazol-2-ylamino)-6-(sulfamoylamino)hexan-2-yl)piperidine-2-carboxamide (265)

The general Procedure B was followed to afford 265 (65 mg, 34%) as a white solid. (CD₃OD) δ (ppm) ¹H, 7.89 (d, J=7.8 Hz, 2H), 7.40-7.36 (m, 3H), 7.29 (t, J=7.4 Hz, 1H), 4.64 (dd, J=9.4, 4.7 Hz, 1Ha), 4.56 (dd, J=9.2, 4.9 Hz, 1Hb), 3.80-3.64 (m, 1H), 3.55-3.43 (m, 1H), 3.16-2.99 (m, 3H), 2.83-2.81 (m, 3H), 2.30-2.18 (m, 1H), 2.00-1.89 (m, 3H), 1.89-1.69 (m, 3H), 1.69-1.35 (m, 6H). LRMS (ESI): (calc) 508.2; (found) 509.2 (MH)⁺.

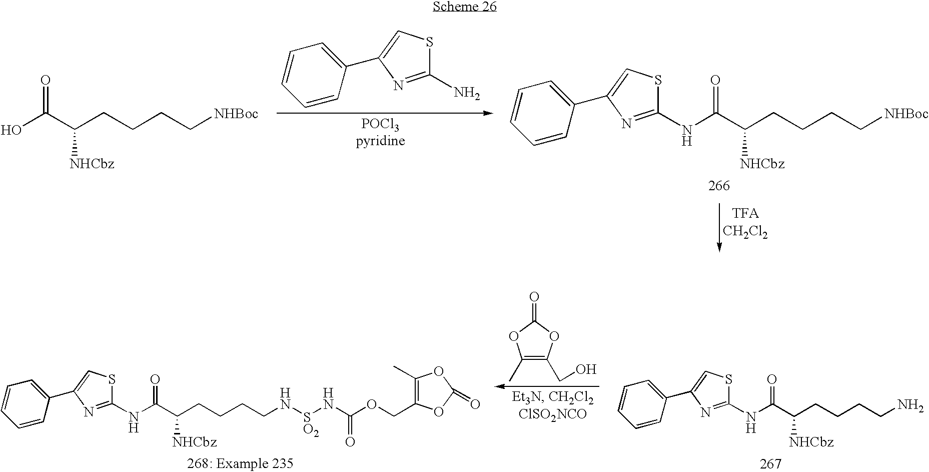

### Example 235

### (S)-2-(benzyloxycarbonylamino)-6-(N-(((5-methyl-2-oxo-1,3-dioxol-4-yl)methoxy)carbonyl)sulfamoylamino)hexanoic acid N-(4-phenylthiazol-2-yl)amide (268)

### Step 1: (S)-2-(benzyloxycarbonylamino)-6-(tert-butoxycarbonylamino)hexanoic acid N-(4-phenylthiazol-2-yl)amide (266)

The general Procedure U was followed to afford 266 (2.25 g, 84%) as a white solid. LRMS (ESI): (calc.) 538.2; (found) 539.3 (MH)⁺.

### Step 2: (S)-benzyl 6-amino-1-oxo-1-(4-phenylthiazol-2-ylamino)hexan-2-ylcarbamate (267)

The general Procedure B was followed to afford 267 as crude colorless oil. LRMS (ESI): (calc) 438.2; (found) 439.1 (MH)⁺.

### Step 3: (S)-2-(benzyloxycarbonylamino)-6-(N-(((5-methyl-2-oxo-1,3-dioxol-4-yl)methoxy)carbonyl)sulfamoylamino)hexanoic acid N-(4-phenylthiazol-2-yl)amide (268)

To a solution of chlorosulfonyl isocyanate (143 uL, 1.65 mmol) in dichloromethane (2 mL) at 0° C. was added 4-(hydroxymethyl)-5-methyl-1,3-dioxol-2-one (215 mg, 1.65 mmol). The mixture was stirred at 0° C. for 10 min, and then for 10 min at room temperature. That intermediate was added dropwise to a mixture of 267 (658 mg, 1.5 mmol) in dichloromethane (4 mL) at 0° C. The mixture was stirred at room temperature for 2 h. Solvent was evaporated, and the residue was purified by silica gel column chromatography with ethyl acetate (80%) in hexanes. The material was purified again by silica gel column chromatography with ethyl acetate (60%) in hexanes, followed by prep-hplc using aquasil C18 column, with acetonitrile (10-95%) in water to afford 268 (80 mg, 8%) as a white solid. (DMSO-d6) δ (ppm) ¹H, 12.40 (s, 1H), 11.33 (s, 1H), 7.89 (d, J=7.4 Hz, 2H), 7.82 (bs, 1H), 7.68 (d, J=7.4 Hz, 1H), 7.63 (s, 1H), 7.42 (t, J=7.4 Hz, 2H), 7.36-7.12 (m, 6H), 5.02 (d, J=2.0 Hz, 2H), 4.97 (s, 2H), 4.25 (q, J=3.5 Hz, 1H), 2.86 (q, J=6.3 Hz, 2H), 2.13 (s, 3H), 1.72-1.54 (m, 2H), 1.52-1.22 (m, 4H). LRMS (ESI): (calc.) 673.2; (found) 674.2 (MH)⁺.

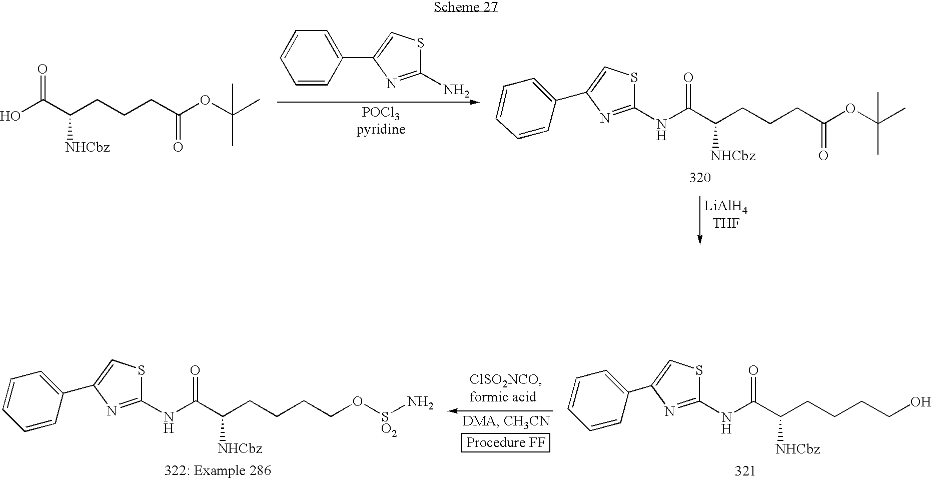

### Example 286

### (S)-5-(benzyloxycarbonylamino)-6-oxo-6-(4-phenylthiazol-2-ylamino)hexyl sulfamate (322)

### Step 1: (S)-tert-butyl 5-(benzyloxycarbonylamino)-6-oxo-6-(4-phenylthiazol-2-ylamino)hexanoate (320)

The general Procedure U was followed to afford 320 (437 mg, 86%) as a white solid. LRMS (ESI): (calc) 509.2; (found) 510.3 (MH)⁺.

### Step 2: (S)-benzyl 6-hydroxy-1-oxo-1-(4-phenylthiazol-2-ylamino)hexan-2-ylcarbamate (321)

The general Procedure S was followed to afford 321 (46 mg, 42%) as a white solid. LRMS (ESI): (calc) 439.2; (found) 440.1 (MH)⁺.

### Step 3: (S)-5-(benzyloxycarbonylamino)-6-oxo-6-(4-phenylthiazol-2-ylamino)hexyl sulfamate (322)

To chlorosulfonyl isocyanate (14 uL, 0.16 mmol) at 0° C. was added formic acid (6 uL, 0.16 mmol). The mixture was stirred until it solidified (5 min). Then acetonitrile (0.15 mL) was added and the mixture was stirred at room temperature for 16 h. The mixture was cooled to 0° C., and then a solution of 321 (46 mg, 0.11 mmol) in dimethylacetamide (0.35 mL) was added. The mixture was stirred at room temperature for 2 h. The mixture was quenched with water and extracted with ethyl acetate. The organic extract was dried (MgSO₄), filtered, and evaporated. The residue was purified by silica gel column chromatography with ethyl acetate (60%) in hexanes to afford 322 (23 mg, 43%) as a white solid. (DMSO-d6) δ (ppm) ¹H, 12.41 (s, 1H), 7.89 (d, J=7.2 Hz, 2H), 7.71 (m, 1H), 7.62 (s, 1H), 7.44-7.13 (m, 10H), 5.02 (d, J=1.6 Hz, 2H), 4.27 (q, J=4.9 Hz, 1H), 4.00 (t, J=6.5 Hz, 2H), 1.78-1.59 (m, 4H), 1.54-1.32 (m, 2H). LRMS (ESI): (calc) 518.1; (found) 519.2 (MH)⁺.

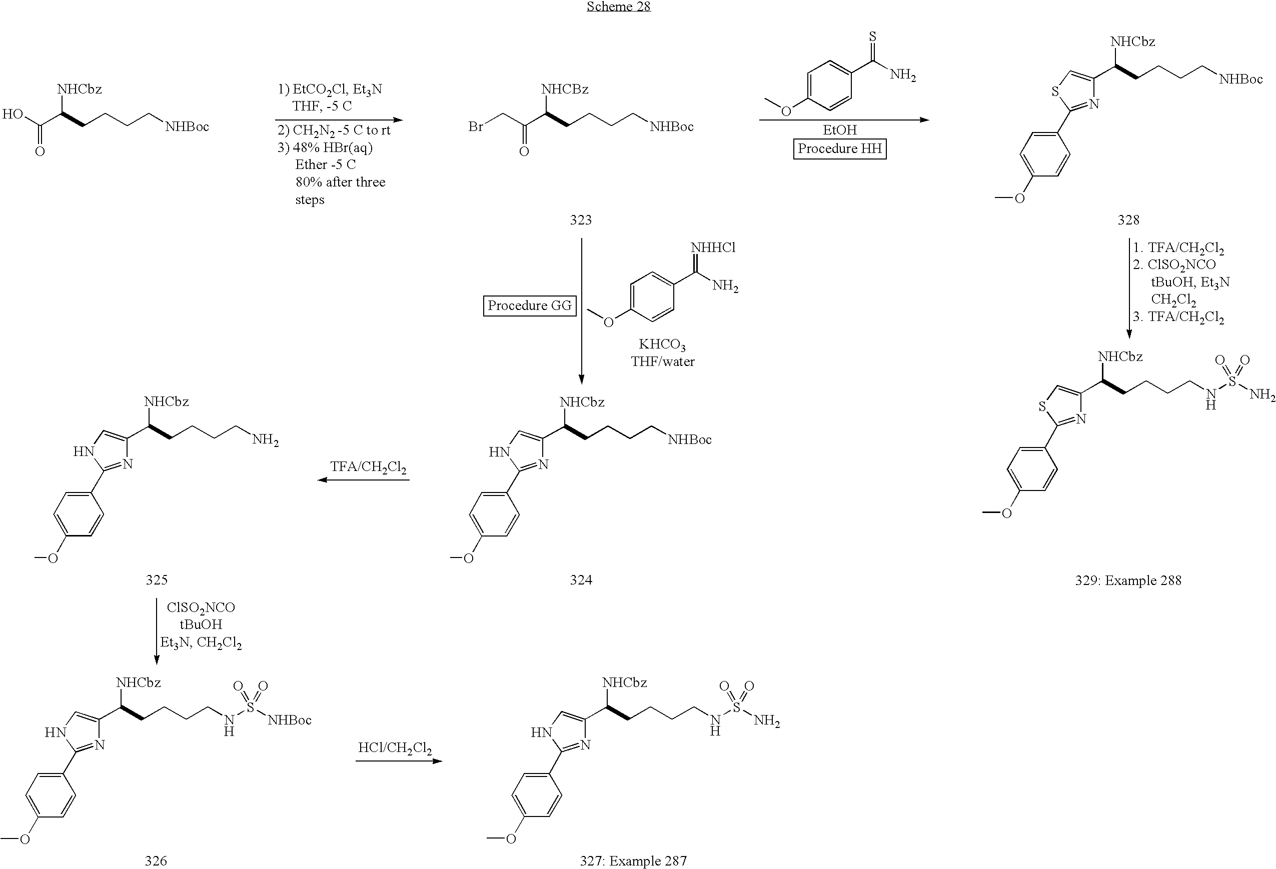

### Example 287

### (S)-benzyl 1-(2-(4-methoxyphenyl)-1H-imidazol-4-yl)-5-(sulfamoylamino)pentylcarbamate (327)

### Step 1: (S)-tert-butyl 5-(benzyloxycarbonylamino)-7-bromo-6-oxoheptylcarbamate (323)

The titled compound was prepared using the procedure reported by G. Abbenante et. al. (J. Am. Chem. Soc. 1995, 117, 10220-10226). To a solution of(S)-2-(benzyloxycarbonylamino)-6-(tert-butoxycarbonylamino)hexanoic acid (1.0 g, 2.63 mmol) in THF (10 mL) under nitrogen was added triethylamine (0.7 mL, 2.89 mmol). The solution was cooled to −10° C. and then ethylchloroformate (0.25 mL, 2.63 mmol) was added. After 30 minutes of stirring at −10° C., an ethereal solution of diazomethane [prepared freshly from N-nitroso-N-methylurea (1.35 g, 13.15 mmol) and KOH (2.21 g, 39.4 mmol) in ether (10 mL)] was added slowly to the reaction at −5° C. over 10 minutes. It was stirred at −5° C. for 30 minutes and then warmed to room temperature over 2 hours. Nitrogen gas was bubbled into the reaction mixture to remove the excess diazomethane. The reaction was diluted with ether and washed with water, NaHCO₃ (sat) and brine and then dried over anhydrous Na₂SO₄. Removal of the ether under reduced pressure gave the diazo-ketone intermediate as yellow oil (1.4 g). LRMS (ESI): (calc) 404.21 (found) 427.3 (MNa)⁺. The diazoketone was dissolved in ether (15 mL) and cooled to −5° C. 48% HBr in water (4×0.10 mL total, 3.54 mmol) was added in four portions over 30 minutes. Progress of the reaction was monitored by TLC, and the reaction was completed 10 minutes after the last addition of HBr. The reaction was diluted with ethyl acetate and washed with saturated NaHCO₃. The aqueous layers were back-extracted with more ethyl acetate. The organic phases were combined and washed with brine and dried over Na₂SO₄. Removal of the solvent gave clear oil which solidified on standing. The solid was purified by silica gel chromatography (Biotage 25M, 25-65% ethyl acetate in hexanes) to give 323 (0.998 g, 83%) as a white solid. LRMS (ESI): (calc) 456.13 (found) 479.1 (MNa)⁺.

### Step 2: (S)-tert-butyl 5-(benzyloxycarbonylamino)-5-(2-(4-methoxyphenyl)-1H-imidazol-4-yl)pentylcarbamate (324)

A mixture of 4-methoxybenzimidamide hydrochloride (229 mg, 1.224 mmol), and potassium hydrogen carbonate (0.161 mL, 3.50 mmol) in THF (2.5 mL) and water (0.4 mL) was heated to reflux then a solution of 323 (400 mg, 0.875 mmol) in 0.6 mL of THF was added over 40 minutes. After 2 hours, the reaction was cooled and the THF removed by evaporation under reduced pressure. The residue was then partitioned between water and DCM. The layers were separated and the water extracted with more DCM (2×). The DCM layers were combined and washed with water, dried over Na₂SO₄, filtered and then purified by silica gel chromatography (Biotage 25M, 30-50% ethyl acetate in DCM) to give 324 (114 mg, 25%) as a white amorphous solid. LRMS (ESI): (calc) 508.2 (found) 509.3 (MH)⁺.

### Step 3: (S)-benzyl 5-amino-1-(2-(4-methoxyphenyl)-1H-imidazol-4-yl)pentylcarbamate (325)

The general procedure B was followed to afford 325 (86 mg, 93%) as a clear film. LRMS (ESI): (calc) 408.2 (found) 409.2 (MH)⁺.

### Step 4: (S)-tert-butyl N-(5-(benzyloxycarbonylamino)-5-(2-(4-methoxyphenyl)-1H-imidazol-4-yl)pentyl)sulfamoylcarbamate (326)

The general procedure M was followed to afford 326 (112.2 mg, 85%) as a white solid. LRMS (ESI): (calc) 587.2 (found) 588.4 (MH)⁺.

### Step 5: (S)-benzyl 1-(2-(4-methoxyphenyl)-1H-imidazol-4-yl)-5-(sulfamoylamino)-pentylcarbamate (327)

The general procedure E was followed to afford 327 (57 mg, 53%) as a crusty white solid after silica gel chromatography (Biotage 12M, 0-10% methanol in DCM).

(CD₃OD) δ (ppm) ¹H, 7.81 (d, J=8.8 Hz, 2H), 7.35-7.27 (m, 5H), 7.21 (s, 1H), 7.09 (d, J=8.8 Hz, 2H), 5.10 (d, J=12.4 Hz, 1H), 5.08 (d, J=12.0 Hz, 2H), 4.77 (m, 1H), 3.87 (s, 3H), 3.03 (t, J=7.2 Hz, 2H), 1.98-1.88 (m, 1H), 1.86-1.74 (m, 1H), 1.66-1.56 (m, 2H), 1.52-1.38 (m, 2H). LRMS (ESI): (calc) 487.2 (found) 488.3 (MH)⁺.

### Example 288

### (S)-benzyl 1-(2-(4-methoxyphenyl)thiazol-4-yl)-5-(sulfamoylamino)pentylcarbamate (329)

### Step 1: (S)-tert-butyl 5-(benzyloxycarbonylamino)-5-(2-(4-methoxyphenyl)thiazol-4-yl)pentylcarbamate (328)

Compound 323 (200 mg, 0.439 mmol) and 4-methoxybenzothioamide (73 mg, 0.439 mmol) were stirred in ethanol (2 mL) for 16 hours at room temperature. Ethanol was then removed under reduced pressure and the residue was dissolved in ethyl acetate and washed with saturated NaHCO₃ (2×), dried over Na₂SO₄, filtered, concentrated and then purified by silica gel chromatography (Biotage 12M, 20-50% ethyl acetate and hexanes) to give 328 (161 mg, 70%) as an off white gum. LRMS (ESI): (calc) 525.2 (found) 526.3 (MH)⁺.

### Step 2: (S)-tert-butyl 5-(benzyloxycarbonylamino)-5-(2-(4-methoxyphenyl)thiazol-4-yl)pentylcarbamate (329)

The general procedures B, M and then B were followed to afford 329 (42 mg, 27%) as a white solid. (DMSO-d₆) δ (ppm) ¹H, 7.84 (d, J=8.8 Hz, 2H), 7.74 (d, J=8.8 Hz, 1H), 7.35-7.26 (m, 5H), 7.28 (s, 1H), 7.02 (d, J=8.8 Hz, 2H), 6.43-6.41 (m, 2H), 5.03 (s, 2H), 4.72-4.67 (m, 1H), 3.80 (s, 3H), 2.83 (t, J=6.0 Hz, 2H), 1.90-1.80 (m, 1H), 1.75-1.63 (m, 1H), 1.53-1.43 (m, 2H), 1.43-1.25 (m, 2H). LRMS (ESI): (calc) 504.2 (found) 505.2 (MH)⁺.

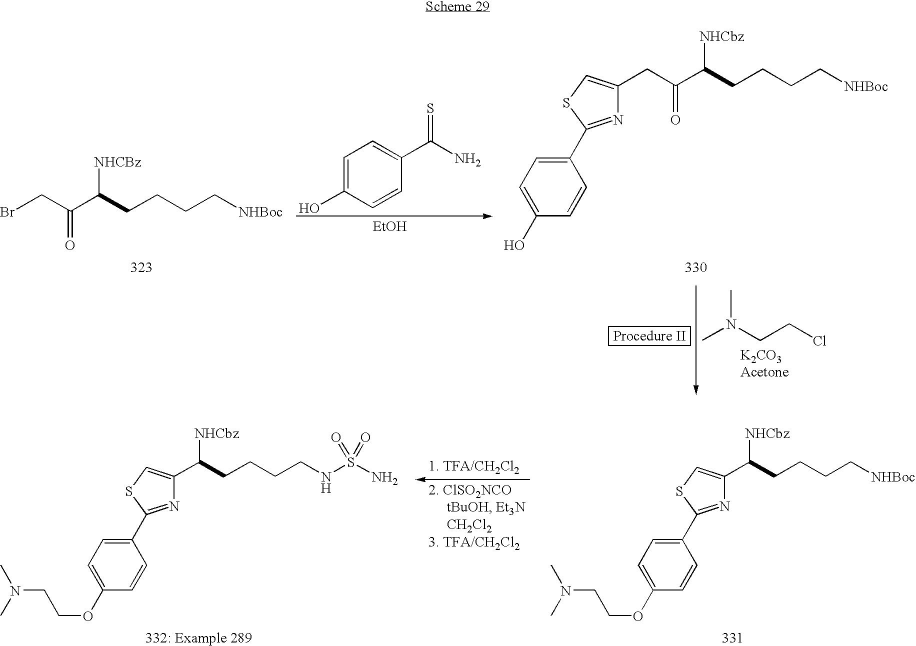

### (S)-benzyl 1-(2-(4-(2-(dimethylamino)ethoxy)phenyl)thiazol-4-yl)-5-(sulfamoylamino)pentyl-carbamate (332)

### Step 1: (S)-tert-butyl 5-(benzylcarboxyamino)-5-(2-(4-hydroxyphenyl)thiazol-4-yl)pentylcarbamate (330)

The general procedure HH was used to provide compound 330 (0.229, 100%). LRMS (ESI): (calc) 511.2 (found) 512.2 (MH)⁺.

### Step 2: (S)-tert-butyl 5-amino-5-(2-(4-(2-(dimethylamino)ethoxy)phenyl)thiazol-4-yl)pentylcarbamate (331)

A mixture of 330 (0.225 g, 0.439 mmol), dimethylaminoethyl chloride HCl (0.063 g, 0.439 mmol), and potassium carbonate (0.121 g, 0.878 mmol) were heated to reflux in acetone (4 mL) for 16 hours. The solids were then filtered and the filtrate concentrated and purified by silica gel chromatography (Biotage 25M column, 5 to 10 to 20% methanol in DCM) to give 331 as a yellow crusty solid (0.124g, 48.5%). LRMS (ESI): (calc) 582.3 (found) 583.5 (MH)⁺.

### Step 3: (S)-benzyl 1-(2-(4-(2-(dimethylamino)ethoxy)phenyl)thiazol-4-yl)-5-(sulfamoylamino)pentyl-carbamate (332)

The general procedures B, M and then B were followed to afford 332 (45 mg, 38%) as a white solid. (DMSO-d₆) δ (ppm) ¹H, 7.82 (d, J=8.8 Hz, 2H), 7.76 (d, J=8.4 Hz, 1H), 7.35-7.20 (m, 5H), 7.28 (s, 1H), 7.03 (d, J=9.2 Hz, 2H), 6.44-6.41 (m, 3H), 5.03 (s, 2H), 4.69 (dt, J=4.8, 8.4 Hz, 1H), 4.09 (t, J=5.6 Hz, 2H), 2.82 (q, J=7.2 Hz, 2H), 2.62 (t, J=5.6 Hz, 2H), 2.20 (s, 6H), 1.88-1.80 (m, 1H), 1.73-1.65 (m, 1H), 1.50-1.42 (m, 2H), 1.40-1.26 (m, 2H). LRMS (ESI): (calc) 561.2 (found) 562.3 (MH)⁺.

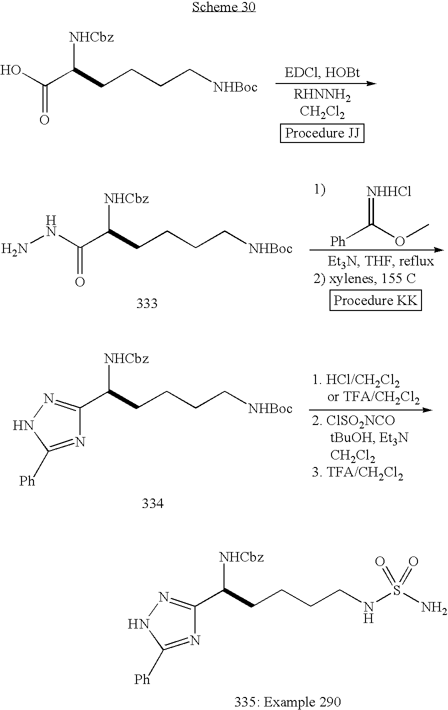

### Example 290

### (S)-benzyl 1-(5-phenyl-1H-1,2,4-triazol-3-yl)-5-(sulfamoylamino)pentylcarbamate (335)

### Step 1: (S)-tert-butyl 5-(benzyloxycarbonylamino)-6-hydrazinyl-6-oxohexylcarbamate (333)

To a suspension of (S)-2-(benzyloxycarbonylamino)-6-(tert-butoxycarbonylamino)hexanoic acid (0.750 g, 1.97 mmol) and HOBt (0.301 g, 1.97 mmol) in dichloromethane (10 mL) was added EDC (0.378 g, 1.97 mmol). The suspension turned to a clear solution after 30 minutes. The solution was cooled in an ice bath, hydrazine monohydrate (0.38 mL, 7.88 mmol) was added and the reaction was stirred in the ice bath for 1 hour, warmed to room temperature and stirred overnight. The dichloromethane was removed by evaporation and the residue redissolved in ethyl acetate. The ethyl acetate was washed with water (3×), saturated NaHCO₃, and brine, dried over Na₂SO₄ and then filtered. Removal of the solvent and silica gel chromatography (Biotage 25M, 5 to 15% methanol in dichloromethane) gave the compound 333 as a hard white solid (0.6312 g, 83%).

LRMS (ESI): (calc) 394.2 (found) 417.3 (MNa)⁺.

### Step 2: (S)-tert-butyl 5-(benzyloxycarbonylamino)-5-(5-phenyl-1H-1,2,4-triazol-3-yl)pentylcarbamate (334)

The reaction was done according to the procedure of B. Li et. al. (Org. Process Res. Dev. 2002, 6, 682-683). Coupling of the acyl hydrazine 333 (0.300 g, 0.761 mmol) and methyl benzimidate HCl (0.130 g, 0.761 mmol) to the acylamidrazones was done by heating a mixture of the two in the presence of triethylamine (0.11 mL, 0.761 mmol) in THF (4 mL) at reflux for 16 hours. The THF was then removed under reduced pressure and the white residue suspended in xylenes (5 mL). The cloudy suspension was heated to reflux for 30 minutes, then the xylenes were removed by evaporation. The residue was dissolved in methanol and dichloromethane, silica gel was added and the solvent evaporated. The brown silica gel was then applied to a 25M Biotage column and eluted with 10% to 30% ethyl acetate in dichloromethane to give 334 (198 mg, 55%) as a light brown solid. LRMS (ESI): (calc) 479.2 (found) 480.2 (MH)⁺.

### Step 3: (S)-benzyl 1-(5-phenyl-1H-1,2,4-triazol-3-yl)-5-(sulfamoylamino)pentyl-carbamate (335)

The general procedures E, M and then B were followed successively to afford 335 (25 mg, 14% over three steps) as a white solid. (CD₃OD) δ (ppm) ¹H, 7.98-7.96 (m, 2H), 7.51-7.46 (m, 3H), 7.38-7.20 (m, 5H), 5.14-5.06 (m, 2H), 4.69 (m in DMSO-d6, 1H), 3.02 (t, J=7.2 Hz, 2H), 2.05-1.97 (m, 1H), 1.95-1.87 (m, 2H), 1.63-1.58 (m, 2H), 1.56-1.43 (m, 2H). LRMS (ESI): (calc) 458.2 (found) 459.2 (MH)⁺.

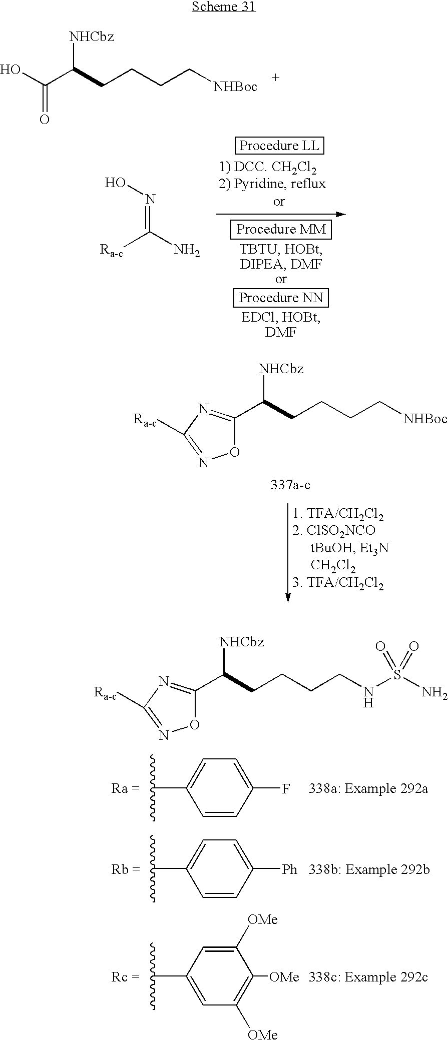

### Example 292a

### (S)-benzyl 1-(3-(4-fluorophenyl)-1,2,4-oxadiazol-5-yl)-5-(sulfamoylamino)pentyl-carbamate (338a)

### Step 1: (S)-tert-butyl 5-(benzyloxycarbonylamino)-5-(3-(4-fluorophenyl)-1,2,4-oxadiazol-5-yl)pentylcarbamate (337a)

The reaction was done following the procedure of S. Borg et al. (J. Org. Chem. 1995 60 3112-3120). The formation of the symmetric anhydride of (S)-2-(benzyloxycarbonylamino)-6-(tert-butoxycarbonylamino)hexanoic acid was done by adding DCC (0.136 g, 0.657 mmol) to a 0° C. solution of (S)-2-(benzyloxycarbonylamino)-6-(tert-butoxycarbonylamino)hexanoic acid (0.500 g, 1.314 mmol) in dichloromethane (5 mL). After 1 hour the resulting dicyclohexyl urea was filtered off and the filtrate concentrated to a white foam then it was redissolved in pyridine (5 mL) and a solution of the 4-fluoro-N′-hydroxybenzimidamide (0.125 g, 0.81 mmol) in pyridine (1 mL) was added. The reaction was then heated to reflux for 3 hours, pyridine was removed by under reduced pressure and the residue dissolved in ethyl acetate. It was then washed with 5% citric acid (aqueous), NaHCO₃ (sat'd), brine, dried over anhydrous sodium sulfate, and then filtered and concentrated. The crude was then purified by silica gel chromatography (Biotage 12M, 20 to 50% ethyl acetate in hexanes) to give 337a (0.136 g, 42%) as a white white solid. LRMS (ESI): (calc) 498.2 (found) 521.3 (MNa)⁺.

### Step 2: (S)-benzyl 1-(3-(4-fluorophenyl)-1,2,4-oxadiazol-5-yl)-5-(sulfamoylamino)pentyl-carbamate (338a)

The general procedures B, M and then B were followed successively to give 338a (35 mg, 28% over three steps) as a white solid. (DMSO-d₆) δ (ppm) ¹H, 8.22 (d, J=7.6 Hz, 1H), 8.05-8.02 (m, 2H), 7.43-7.37 (m, 2H), 7.36-7.29 (m, 5H), 6.45 (m, 3H), 5.05 (s, 2H), 4.94-4.88 (m, 1H), 2.84 (q, J=6.0 Hz, 2H), 1.97-1.80 (m, 2H), 1.50-1.30 (m, 4H).

LRMS (ESI): (calc) 477.15 (found) 478.3 (MH)⁺.

### Example 292b

### (S)-benzyl 1-(3-(biphenyl-4-yl)-1,2,4-oxadiazol-5-yl)-5-(sulfamoylamino)pentylcarbamate (338b)

### Step 1: (S)-tert-butyl 5-(benzyloxycarbonylamino)-5-(3-(biphenyl-4-yl)-1,2,4-oxadiazol-5-yl)pentylcarbamate (337b)

The reaction was done following the procedure of R. F. Poulain et al. (Tetrehedron Letters 2001, 42, 1495-1498). To a solution of the (S)-2-(benzyloxycarbonylamino)-6-(tert-butoxycarbonylamino)hexanoic acid (0.400 g, 1.05 mmol), TBTU (0.337 g, 1.05 mmol), HOBt (32 mg, 0.21 mmol) and DIPEA (0.44 mL, 5.25 mmol) in DMF (5 mL) was added the N′-hydroxybiphenyl-4-carboximidamide (0.222 g, 1.05 mmol). The reaction was stirred at room temperature for 1 hour and then heated to 110° C. for 2 hours. The reaction mixture was cooled to room temperature and diluted with water (100 mL) and extracted with ethyl acetate. The combined organic phases were then washed with 1N HCl(aq), NaHCO₃ (sat'd), brine and dried over anhydrous sodium sulfate. The solvent was removed and the crude purified by silica gel chromatography (Biotage 25M column, 20-50% ethyl acetate in hexanes) to give 337b (0.431 g, 74%) as a white foam. LRMS (ESI): (calc) 556.2 (found) 579.4 (MNa)⁺.

### Step 2: (S)-benzyl 1-(3-(4-fluorophenyl)-1,2,4-oxadiazol-5-yl)-5-(sulfamoylamino)pentyl-carbamate (338b)

The general procedures B, M and then B were followed successively to give 338b (119 mg, 29% over three steps) as a white solid. (DMSO-d₆) δ (ppm) ¹H, 8.23 (d, J=8.4 Hz, 1H), 8.06 (d, J=8.4 Hz, 2H), 7.87 (d, J=8.8 Hz, 2H), 7.74 (d, J=8.4 Hz, 2H), 7.52-7.48 (m, 2H), 7.43-7.39 (m, 1H), 7.39-7.31 (m, 5H, shows 4H because of broadening), 6.45 (m, 3H), 5.07 (m, 2H), 4.97-4.90 (m, 1H), 2.84 (q, J=6.4 Hz, 2H), 1.97-1.80 (m, 2H), 1.55-1.30 (m, 4H). LRMS (ESI): (calc) 535.13 (found) 536.3 (MH)⁺.

### Example 292c

### (S)-benzyl 5-(sulfamoylamino)-1-(3-(3,4,5-trimethoxyphenyl)-1,2,4-oxadiazol-5-yl)pentyl-carbamate (338c)

### Step 1: (S)-tert-butyl 5-(benzyloxycarbonylamino)-5-(3-(3,4,5-trimethoxyphenyl)-1,2,4-oxadiazol-5-yl)pentylcarbamate (337c)

In a 15 mL pressure tube, (S)-2-(benzyloxycarbonylamino)-6-(tert-butoxycarbonyl-amino)hexanoic acid (0.300 g, 0.789 mmol), EDCl (0.151 g, 0.789 mmol), and HOBT (0.119 g, 0.789 mmol) were stirred at room temperature in DMF (5 mL) for 30 minutes. N′-hydroxy-3,4,5-trimethoxybenzimidamide (0.178 g, 0.789 mmol) was then added and the reaction heated to 140° C. for 2 hours. The reaction was then diluted with water (˜100 mL) and extracted into ethyl acetate (4×). The organic phases were combined and washed with brine, dried over anhydrous sodium sulfate and concentrated to an oil and purified by silica gel chromatography (Biotage 25M, 20 to 60% ethyl acetate in hexanes) to give 337c (0.368 g, 82%) as a white foam. LRMS (ESI): (calc) 570.2 (found) 593.2 (MNa)⁺.

### Steps 2-4: (S)-benzyl 1-(3-(4-fluorophenyl)-1,2,4-oxadiazol-5-yl)-5-(sulfamoylamino)pentyl-carbamate (338c)

The general procedures B, M and then B were followed successively to give 338c (59 mg, 16% over three steps) as a white solid. (DMSO-d₆) δ (ppm) ¹H, 8.24 (d, J=7.2 Hz, 1H), 7.36-7.30 (m, 5H, shows 4H because of broadening), 7.23 (s, 2H), 6.45 (s, 3H), 5.05 (m, 2H), 4.94-4.84 (m, 1H), 3.84 (s, 6H), 3.72 (s, 3H), 2.84-2.83 (m, 2H), 1.97-1.80 (m, 2H), 1.51-1.30 (m, 4H). LRMS (ESI): (calc) 549.19 (found) 550.2 (MH)⁺.

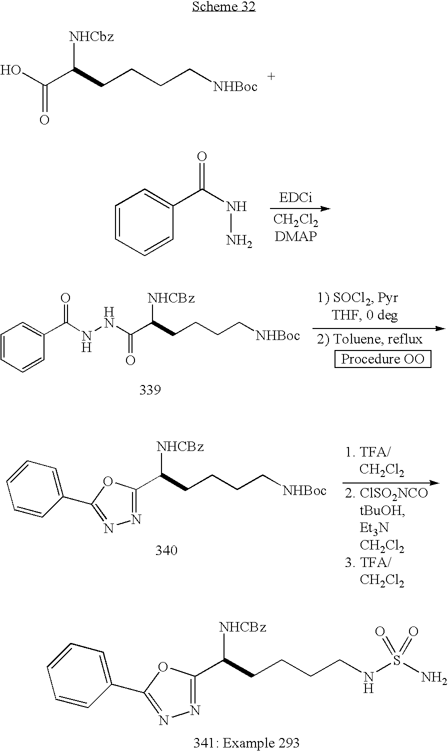

### Example 293

### (S)-benzyl 1-(5-phenyl-1,3,4-oxadiazol-2-yl)-5-(sulfamoylamino)-pentylcarbamate (341)

### Step 1: (S)-tert-butyl 5-(benzyloxycarbonylamino)-6-(2-benzoylhydrazinyl)-6-oxohexylcarbamate (339)

The general procedure JJ was used to generate compound 339 (1.29 g, 99%) as a white solid. LRMS (ESI): (calc) 498.2 (found) 499.5 (MH)⁺.

### Step 2: (S)-tert-butyl 5-(benzyloxycarbonylamino)-5-(5-phenyl-1,3,4-oxadiazol-2-yl)pentylcarbamate (340)

The reaction was done following the procedure of S. Borg et al. (J. Org. Chem. 1995 60 3112-3120). To a solution of 339 (0.400 g, 0.802 mmol) in THF (5 mL), cooled to 0° C. and under nitrogen was added thionyl chloride (0.081 mL, 1.04 mmol) followed by pyridine (0.16 mL, 2.00 mmol). The reaction was stirred at 0° C. for 2 hours. It was then filtered and the filtrate concentrated and the resulting oil was suspended in toluene (5 mL) and then heated to reflux for 1.5 hours. The toluene was then removed by evaporation under reduced pressure and the residue purified twice by silica gel chromatography (Biotage 25M, 20 to 50% ethyl acetate in hexanes) to give 340 (110 mg, 29%) as a clear gum that was about 75% pure by proton NMR. LRMS (ESI): (calc) 480.2 (found) 481.2 (MH)⁺.

### Step 3: (S)-benzyl 1-(5-phenyl-1,3,4-oxadiazol-2-yl)-5-(sulfamoylamino)pentylcarbamate (341)

The general procedures B, M and then B were followed to give 341 (13.7 mg, 15% over 3 steps) as a white solid. (DMSO-d₆) δ (Ppm) ¹H, 8.13 (d, J=8.0 Hz, 1H), 7.93 (d, J=8.4 Hz, 2H), 7.63-7.57 (m, 3H), 7.36-7.30 (m, 5H, shows 4H because of broadening), 6.45 (m, 3H), 5.05 (m, 2H), 4.94-4.84 (m, 1H), 2.85 (q, J=6.4 Hz, 2H), 2.00-1.80 (m, 2H), 1.53-1.30 (m, 4H). LRMS (ESI): (calc) 459.16 (found) 460.2 (MH)⁺.

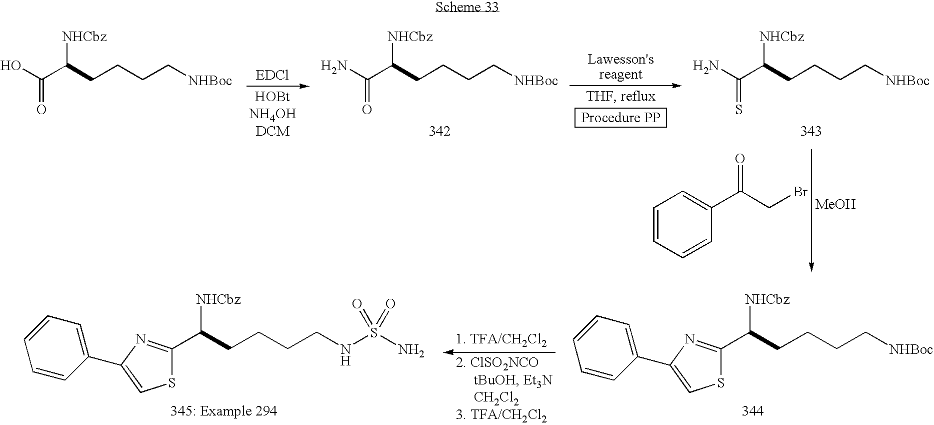

### Example 294

### (S)-benzyl 1-(4-phenylthiazol-2-yl)-5-(sulfamoylamino)pentylcarbamate (345)

### Step 1: (S)-tert-butyl 6-amino-5-(benzyloxycarbonylamino)-6-oxohexylcarbamate (342)

The general procedure JJ was used to afford 342 (2.00 g, 76%) as a white solid. LRMS (ESI): (calc) 379.2 (found) 402.2 (MNa)⁺.

### Step 2: (S)-tert-butyl 6-amino-5-(benzylcarbonylamino)-6-thioxohexylcarbamate (343)

A solution of compound 342 (2.00 g, 5.26 mmol) and Lawessons' reagent (2.34 g, 5.8 mmol) in THF (26 mL) was heated to reflux for 1 hour. The reaction mixture was then concentrated under reduced pressure and purified by silica gel chromatography (25-60% ethyl acetate in hexanes) to give 343 (1.71 g, 83%) as a white solid. LRMS (ESI): (calc) 395.2 (found) 418.2 (MNa)⁺.

### Step 3: (S)-tert-butyl 5-(benzyloxycarbonylamino)-5-(4-phenylthiazol-2-yl)pentylcarbamate (344)

Compound 343 (0.232 g, 0.587 mmol) and F-bromoacetophenone (0.116g, 0.587 mmol) were reacted following the general procedure HH to give product 344 (0.100 g, 34%) as a white solid. LRMS (ESI): (calc) 495.2 (found) 496.3 (MNa)⁺.

### Step 4: (S)-benzyl 1-(4-phenylthiazol-2-yl)-5-(sulfamoylamino)pentylcarbamate (345)

The general procedures B, M and then B were followed successively to give 345 (14 mg, 15% over 3 steps) as a white solid. (CD₃OD) δ (ppm) 1H, 7.80 (d, 2H, J=8.4 Hz), 7.57 (s, 1H), 7.18-7.33 (m, 8H), 5.04 (s, 2H), 4.90-4.95 (m, 1H), 2.94 (t, 2H, J=7.0 Hz), 2.00-2.08 (m, 1H), 1.75-1.84 (m, 1H), 1.39-1.59 (m, 4H). LRMS (ESI): (calc.) 474.60 (found) 475.2 (MH)⁺.

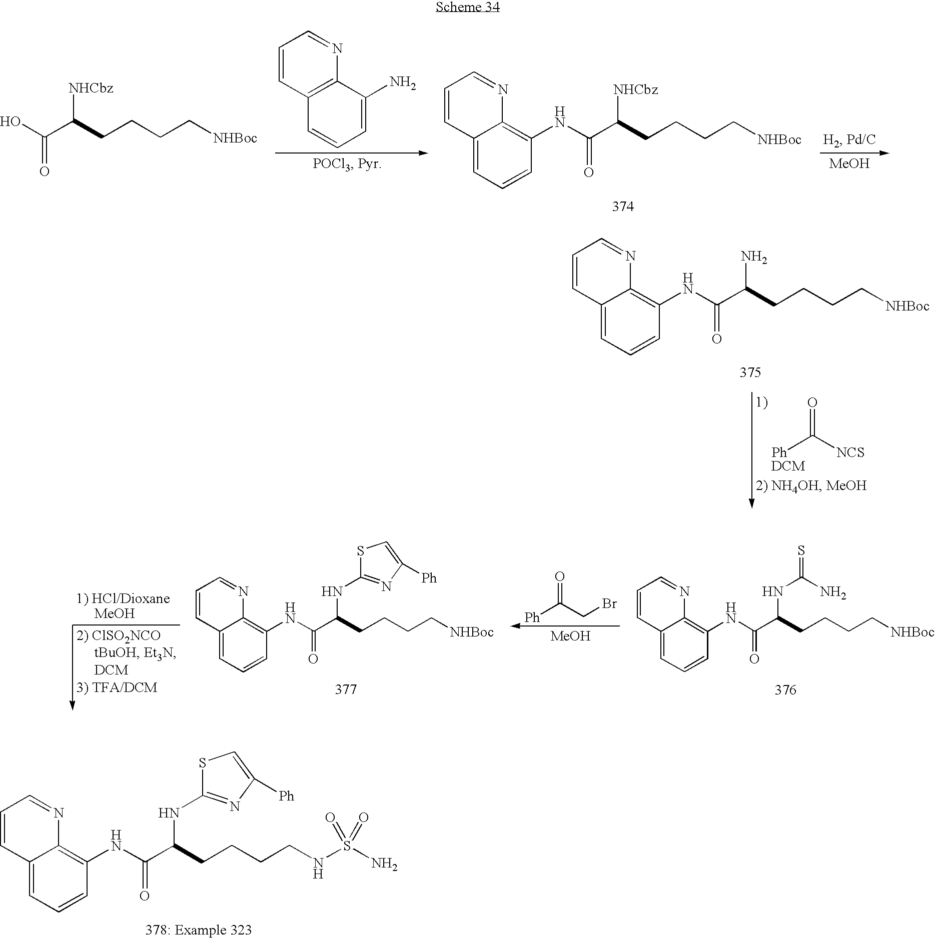

### Example 323

### (S)-2-(4-phenylthiazol-2-ylamino)-N-(quinolin-8-yl)-6-(sulfamoylamino)hexanamide (378)

### Step 1: (S)-tert-butyl 5-(benzyloxycarbonylamino)-6-oxo-6-(quinolin-8-ylamino)hexylcarbamate (374)

The general procedure U was used to give the titled compound 374 (2.73 g, 51%) as a white solid. LRMS (ESI): (calc) 506.2 (found) 507.4 (MH)⁺.

### Step 2: (S)-tert-butyl 5-amino-6-oxo-6-(quinolin-8-ylamino)hexylcarbamate (375)

The general procedure C was used to give the titled compound 375 (0.602 g, 82%) as a white solid. LRMS (ESI): (calc) 372.2 (found) 373.3 (MH)⁺.

### Step 3: (S)-tert-butyl 6-oxo-6-(quinolin-8-ylamino)-5-thioureidohexylcarbamate (376)

To a solution of 375 (0.602 g, 1.62 mmol) in DCM (8.0 mL) was added benzoylthioisocyanate (0.26 mL, 1.94 mmol). The reaction was stirred at room temperature for 1 hour, then DCM was removed under reduced pressure and the residue re-dissolved in methanol (8.0 mL) and ammonium hydroxide (4.5 mL). The mixture was stirred for 16 hours at room temperature. It was then concentrated and purified by silica gel flash chromatography (50 to 75% ethyl acetate in hexanes) to give 376 (0.275 g, 36%) as a brown solid. LRMS (ESI): (calc) 431.2 (found) 432.3 (MH)⁺.

### Step 4: (S)-tert-butyl 6-oxo-5-(4-phenylthiazol-2-ylamino)-6-(quinolin-8-ylamino)hexyl-carbamate (377)

The general procedure HH was employed to furnish 377 (0.338 g, 70%) as a red oil. LRMS (ESI): (calc) 531.2 (found) 532.3 (MH)⁺.

### Step 5: (S)-2-(4-phenylthiazol-2-ylamino)-N-(quinolin-8-yl)-6-(sulfamoylamino)-hexanamide (378)

The general procedures E, M, and then B were followed to synthesize 378 (1.4 mg, 0.6% over three steps) as a white solid. (CD₃OD) δ (ppm) 1H, 8.66-8.69 (m, 2H), 8.24 (dd, 1H, J=1.7, 8.4 Hz), 7.75-7.78 (m, 2H), 7.46-7.60 (m, 3H), 7.15-7.24 (m, 3H), 6.88 (s, 1H), 4.64-4.67 (m, 1H), 3.06 (t, 2H, J=6.4 Hz), 2.10-2.18 (m, 1H), 1.81-1.97 (m, 1H), 1.59-1.68 (m, 4H). LRMS (ESI): (calc) 510.15 (found) 511.1 (MH)⁺.

The general procedures A to QQ used to synthesize compounds of this invention are described in the Table 1. A specific example of each general procedure is provided in the indicated step of a particular example.

TABLE 1

| Procedure | Scheme | Ex. | Step | Reaction conditions |
|---|---|---|---|---|
| A | 1 | 1 | 1 | 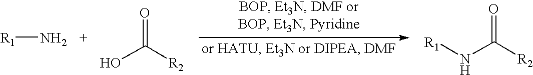 |
| B | 1 | 1 | 2 | 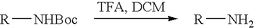 |
| | | | | 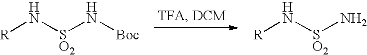 |
| C | 1 | 1 | 3 | 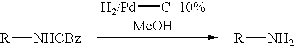 |
| D | 1 | 2a | 1 | 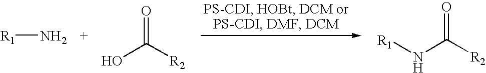 |
| E | 1 | 2a | 2 | 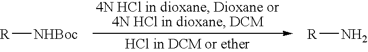 |
| F | 1 | 2a | 3 | 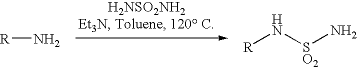 |
| G | 2 | 3 | 2 | 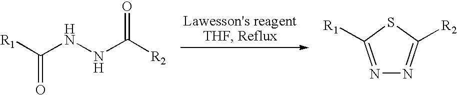 |
| H | 2 | 3 | 5 | 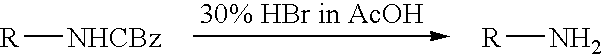 |
| I | 4 | 5 | 4 | 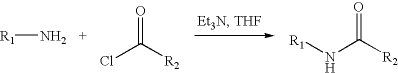 |
| J | 5 | 6 | 2 | 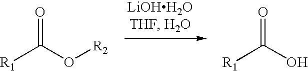 |
| K | 4 | 6 | 3 | 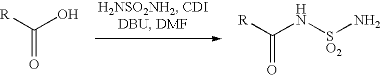 |
| L | 6 | 7 | 9 | 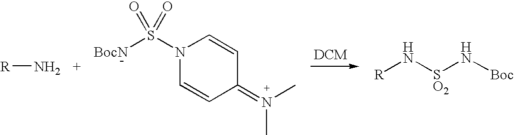 |
| M | 7 | 8 | 1 | 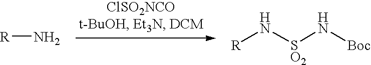 |
| N | 1 | 1c | 1 | 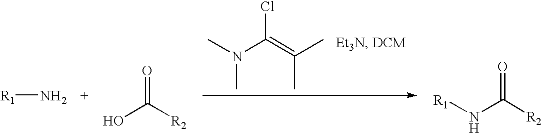 |
| O | 1 | 1c | 3 | 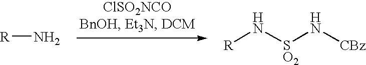 |
| P | 3 | 4 | 1 | 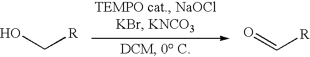 |
| Q | 4 | 5 | 2 | 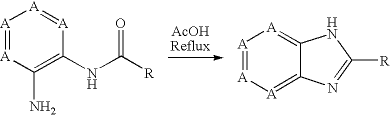 |
| R | 7 | 8 | 5 | 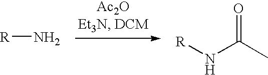 |
| S | 8 | 9 | 2 | 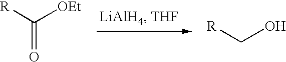 |
| T | 14 | 201 | 1 | 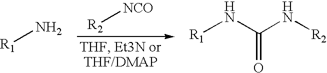 |
| U | 16 | 209 | 1 | 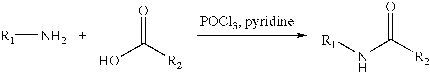 |
| V | 17 | 212 | 1 | 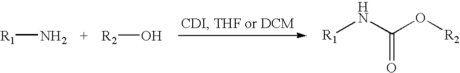 |
| W | 13 | 200 | 1 | 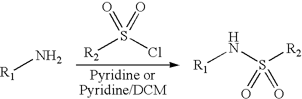 |
| X | 15 | 202 | 1 | 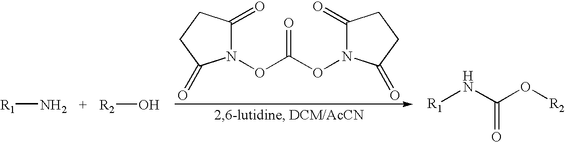 |
| Y | 19 | 213 | 4 | 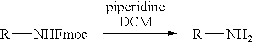 |
| Z | 21 | 217 | 1 | 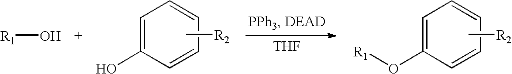 |
| AA | 22 | 230 | 1 | 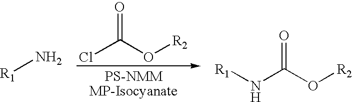 |
| BB | 23 | 231 | 1 | 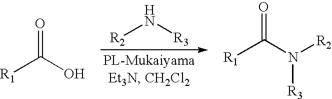 |
| CC | 24 | 232 | 1 | 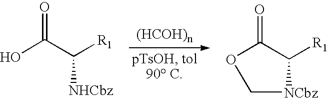 |
| DD | 24 | 232 | 2 | 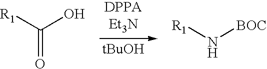 |
| EE | 25 | 234 | 1 | 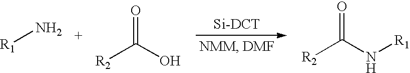 |
| FF | 27 | 286 | 3 | 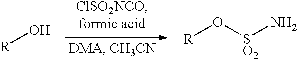 |
| GG | 28 | 287 | 2 | 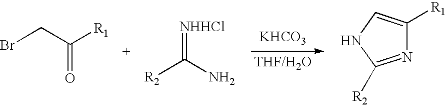 |
| HH | 28 | 288 | 1 | 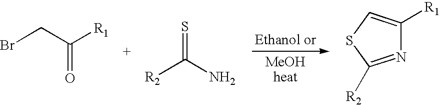 |
| II | 29 | 289 | 2 | 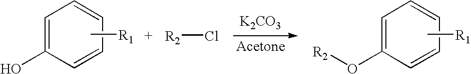 |
| | | | | 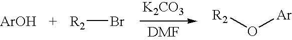 |
| JJ | 30 | 290 | 1 | 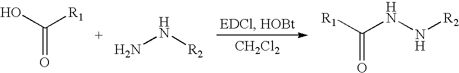 |
| KK | 30 | 290 | 2 | 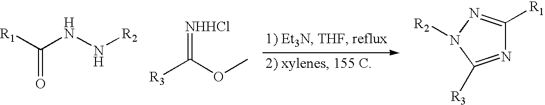 |
| LL | 31 | 292a | 1 | 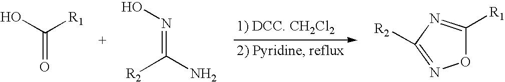 |
| MM | 31 | 292b | 1 | 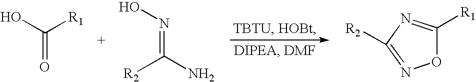 |
| NN | 31 | 292c | 1 | 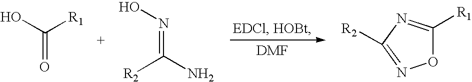 |
| OO | 32 | 293 | 2 | 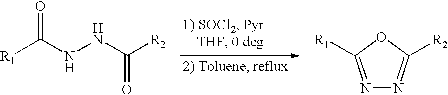 |
| PP | 33 | 294 | 2 | 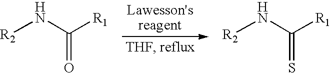 |
| QQ | 9 | 10 | 3 | 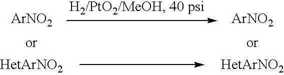 |

The compounds 38-379 described in this invention, Table 2, are prepared starting from the indicated starting material and following the given preparative sequence(s) utilizing the general procedures listed in Table 1.

TABLE 2

| | | | | | | Prep- |
|---|---|---|---|---|---|---|
| | | | | | | ara- |
| | | | | | | tive |
| | | | | | | se- |
| Ex | Cpd | Starting Material | Structure | Name | Characterization | quence |
| 39 | 38 | 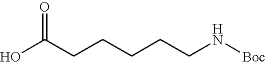 | 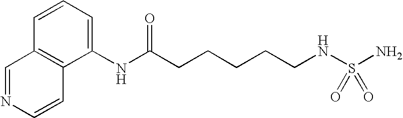 | N-(isoquinolin- 5-yl)-6- (sulfamoyl- amino)- hexanamide | (DMSO-d6) δ (ppm): 9.98 (s, 1 H), 9.30 (s, 1 H), 8.53 (d, J = 5.9 Hz, 1 H), 7.99 (d, J = 7.5 Hz, 1 H), 7.98-7.90 (m, 2 H), 7.65 (t, J = 7.8 Hz, 1 H), 6.46 (s, 2 H), 6.39 (s, 1 H), 2.92-2.83 (m, 2 H), 2.53-2.44 (m, 2 H + dmso), 1.70-1.60 (m, 2 H), 1.58-1.46 (m, 2 H), 1.44-1.34 (m, 2 H). LRMS: 336.13 (calc) 337.18 | |
| # (found) (MH)+ | A, E, F | | | | | |
| 10 | 39 | 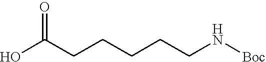 | 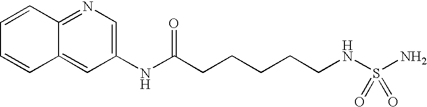 | N-(quinolin-3- yl)-6- (sulfamoyl- amino)- hexanamide | (MeOD-d4) δ (ppm): 8.89 (d, J = 2.5 Hz, 1 H), 8.71 (d, J = 2.6 Hz, 1 H), 7.96 (d, J = 8.4 Hz, 1 H), 7.86 (d, J = 7.2 Hz, 1 H), 7.69-7.65 (m, 1 H), 7.60-7.56 (m, 1 H), 3.31 (t, J = 7.1 Hz, 2 H), 2.49 (t, J = 7.5 Hz, 2 H), 1.82-1.72 (m, 2 H), 1.68-1.59 (m, 2 H), 1.54-1.44 (m, 2 H). LRMS: 336.13 (calc) 335.12 (found) | |
| # (MH)+ | A, E, F | | | | | |
| 11 | 40 | 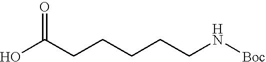 | 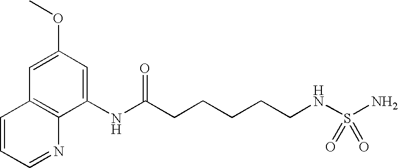 | N-(6- methoxy- quinolin-8- yl)-6- (sulfamoyl- amino)- hexanamide | (MeOD-d4) δ (ppm): 8.66 (dd, J = 4.1, 1.6 Hz, 1 H), 8.31 (d, J = 2.7 Hz, 1 H), 8.15 (dd, J = 8.3, 1.7 Hz, 1 H), 7.46 (dd, J = 8.3, 4.2 Hz, 1 H), 6.95 (d, J = 2.5 Hz, 1 H), 3.05 (t, J = 7.1 Hz, 2 H), 2.59 (t, J = 7.5 Hz, 2 H), 1.84-1.75 (m, 2 H), 1.68-1.59 (m, 2 H), 1.54-1.45 (m, 2 H). LRMS: | |
| # 366.14 (calc) 365.19 (found) (MH)+ | A, E, F | | | | | |
| 12 | 41 | 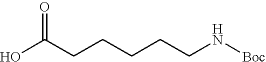 | 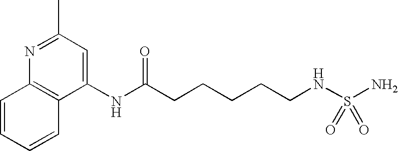 | N-(2- methyl- quinolin-4- yl)-6- (sulfamoyl- amino)- hexanamide | (MeOD-d4) δ (ppm): 8.13 (d, J = 8.4 Hz, 1 H), 8.01 (s, 1 H), 7.92 (d, J = 8.4 Hz, 1 H), 7.71 (t, J = 7.1 Hz, 1 H), 7.54 (t, J = 7.6 Hz, 1 H), 3.06 (t, J = 7.1 Hz, 2 H), 2.67 (s, 3 H), 2.61 (t, 7.4 Hz, 2 H), 1.84-1.74 (m, 2 H), 1.68-1.59 (m, 2 H), 1.55-1.45 (m, 2 H). LRMS: 350.14 (calc) 349.19 (found) | |
| # (MH)+ | A, E, F | | | | | |
| 13 | 42 | 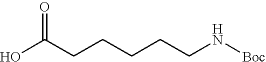 | 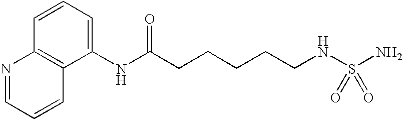 | N-(quinolin- 5-yl)-6- (sulfamoyl- amino)- hexanamide | (MeOD-d4) δ (ppm): 8.76 (d, J = 3.7 Hz, 1 H), 8.35 (d, J = 8.4 Hz, 1 H), 7.84 (d, J = 8.3 Hz, 1 H), 7.68 (t, J = 7.9 Hz, 1 H), 7.61 (d, J = 7.4 Hz, 1 H), (dd, J = 8.5, 4.2 Hz, 1 H), 2.99 (t, J = 6.9 Hz, 2 H), 2.48 (t, J = 7.4 Hz, 2 H), 1.78-1.68 (m, 2 H), 1.62-1.52 (m, 2 H), 1.51-1.41 (m, 2 H). LRMS: | |
| # 336.13 (calc) 335.09 (found) (MH)+ | A, E, F | | | | | |
| 14 | 43 | 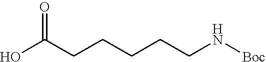 | 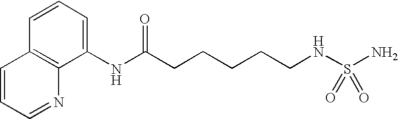 | N-(quinolin- 8-yl)-6- (sulfamoyl- amino)- hexanamide | (MeOD-d4) δ (ppm): 8.90 (dd, J = 4.1, 1.6 Hz, 1 H), 8.65 (d, J = 7.4 Hz, 1 H), 8.33 (dd, J = 8.2, 1.4 Hz, 1 H), 7.68-7.63 (m, 1 H), 7.61-7.54 (m, 2 H), 3.09 (t, J = 6.8 Hz, 2 H), 2.65 (t, J = 7.4 Hz, 2 H), 1.90-1.80 (m, 2 H), 1.73-1.63 (m, 2 H), 1.60-1.50 (m, 2 H). LRMS: 336.4 (calc) 337.1 (found) (MH)+ | A, E, F |
| 15 | 44 | 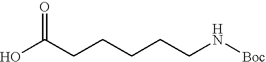 | 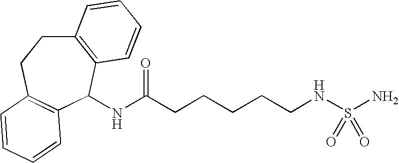 | N-(10, 11- dihydro- 5H- dibenzo[a,a]- cyclo- hepten-5-yl)-6- (sulfamoyl- amino)- hexanamide | (MeOD-d4) δ (ppm): 7.37-7.33 (m, 2 H), 7.19-7.11 (m, 6 H), 3.38-3.28 (m, 2 H), 3.16-3.06 (m, 2 H), 2.96 (t, J = 7.2 Hz, 2 H), 2.28 (t, J = 7.3 Hz, 2 H), 1.66-1.57 (m, 2 H), 1.56-1.48 (m, 2 H), 1.37-1.28 (m, 2 H). LRMS: 401.18 (calc), 400.16 (found) (MH)+ | A, E, F |
| 16 | 45 | 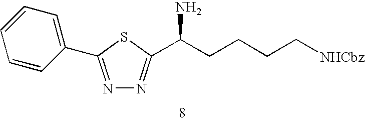 | 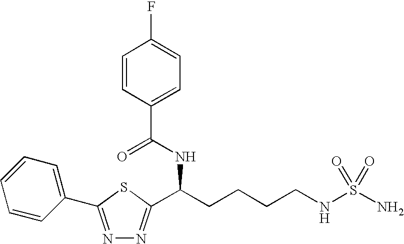 | (S)-4-fluoro- N-(1- (5-phenyl- 1,3,4- thiadiazol-2- yl)-5- (sulfamoyl- amino)- pentyl)- benzamide | (MeOD-d4) δ (ppm) ¹H: 8.20-7.40 (m, 4 H), 7.60-7.50 (m, 3 H), 7.29-7.22 (m, 2 H), 5.61 (dd, J = 9.4, 5.7 Hz, 1 H), 3.11 (t, J = 6.7 Hz, 2 H), 2.39-2.19 (m, 2 H), 1.78-1.56 (m, 4 H). LRMS (ESI): (calc) 463.5; (found) 464.3. (MH)+ | A, H, F |
| 17 | 46 | 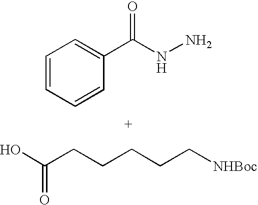 | 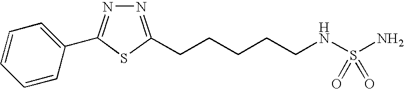 | 5-(5-phenyl- 1,3,4- thiadiazol-2- yl)pentan-1- sulfamate | (DMSO-d6) δ (ppm) ¹H: 8.02-7.94 (m, 1 H), 7.63-7.55 (m, 2 H), 6.56-6.42 (m, 5 H), 3.17 (t, J = 7.4 Hz, 2 H), 2.94-2.86 (m, 2 H), 1.86-1.75 (m, 2 H), 1.61-1.38 (m, 4 H). LRMS (ESI): (calc) 326.4; (found) 327.0. (MH)+ | A, G, B, F |
| 18 | 47 | 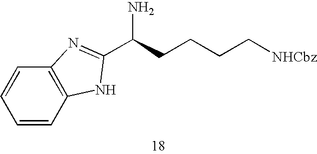 | 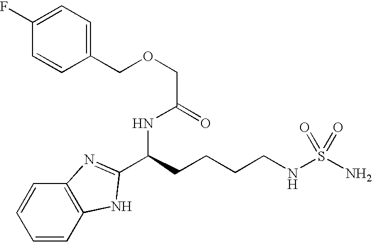 | (S)-N-(1-(1H- benzo[d]- imidazol- 2-yl)-5- (sulfamoyl- amino)- pentyl)-2-(4- fluoro- benzyloxy)- acetamide | (MeOD-d₄) δ (ppm) ¹H: 7.53 (bs, 1 H), 7.42 (t, J = 8.8 Hz, 2 H), 7.22 (m, 2 H), 7.06 (t, J =8.8 Hz, 2 H), 5.25 (t, J = 8.8 Hz, 1 H), 4.61 (s, 2 H), 4.05 (s, 2 H), 3.02 (t, J = 6.8 Hz, 2 H), 2.05 (m, 2 H), 1.63-1.24 (m, 4 H), LRMS: (calc.) | |
| # 463.1; (found) 464.3 (MH)⁻. | A, C, L, E | | | | | |
| 19 | 48 | 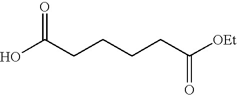 | 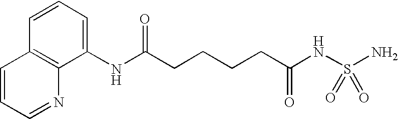 | N¹-(quinolin- 8-yl)- N6- sulfamoyl- adipamide | (DMSO-d₆) δ (ppm): 11.33 (s, 1 H), 10.04 (s, 1 H), 8.91 (m, 1 H), 8.60 (d, J = 7.0 Hz, 1 H), 8.39 (d, J = 8.2 Hz, 1 H), 7.63 (m, 2 H), 7.55 (t, J = 7.8 Hz, 1 H), 7.28 (bs, 2 H), 2.57 (t, J = 6.6 Hz, 2 H), 2.22 (t, J = 7.2 Hz, 2 H), 1.61 (m, 4 H). LRMS: (calc) 350.1, (found) 351.1 (MH)+ | A, J, K |
| 20 | 49 | 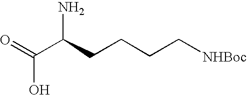 | 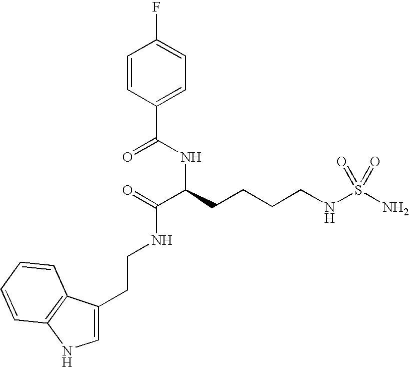 | (S)-N-(1-(2- (1H-indol-3- yl)ethylamino)- 1-oxo-6- (sulfamoyl- amino)- hexan-2-yl)-4- fluoro- benzamide | (MeOD-d₄) δ (ppm) ¹H: 7.88 (t, J = 5.6 Hz, 2 H), 7.56 (d, J = 8.0 Hz, 1 H), 7.31 (d, J =8.0 Hz, 1 H), 7.18 (t, J = 8.4 Hz, 2 H), 7.06 (m, 2 H), 6.97 (t, J = 7.6 Hz, 1 H), 4.45 (t, J = 8 Hz, 1 H), 3.53 (m, 2 H), 2.98 (m, 4 H), 1.75 (m, 2 H), 1.56 | |
| # (m, 2 H), 1.39 (m, 2 H). LRMS: (calc) 489.1; (found) 488.3 (M − H)⁻. | I, A, E, L, E | | | | | |
| 21 | 50 | 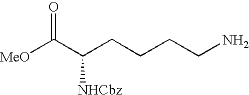 | 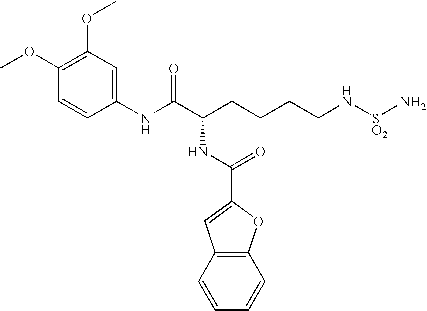 | (S)-N-(1-(3,4- dimethoxy- phenylamino)- 1-oxo-6- (sulfamoyl- amino)- hexan-2-yl)- benzofuran-2- carboxamide | (DMSO-d₆) δ (ppm) ¹H: 9.99 (s, 1 H), 8.68 (d, J = 7.8 Hz, 1 H), 7.77 (d, J = 7.8 Hz, 1 H), 7.68 (d, J = 8.4 Hz, 1 H), 7.64 (s, 1 H), 7.46 (t, J = 7.4 Hz, 1 H), 7.33 (t, J = 7.6 Hz, 1 H), 7.31 (d, J = 2.3 Hz, 1 H), 7.11 (dd, J = 8.8, 2.3 Hz, | |
| # 1 H), 6.87 (d, J = 8.8 Hz, 1 H), 6.46-6.43 (m, 3 H), 4.54 (q, J = 7.4 Hz, 1 H), 3.70 (s, 3 H), 3.69 (s, 3 H), 2.85 (q, J = 6.3 Hz, 2 H), 1.81 (q, J = 7.2 Hz, 2 H), 1.59-1.32 (m, 4 H). LRMS (ESI): (calc.) 504.2; (found) 505.2 (MH)⁺. | M, C, A, J, D, B | | | | | |
| 22 | 51 | 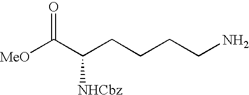 | 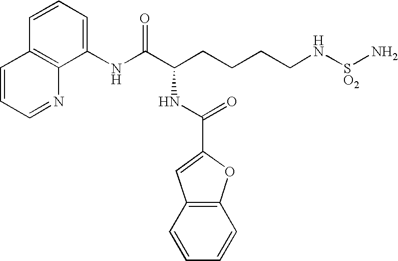 | (S)-N-(1-oxo- 1-(quinolin-8- ylamino)-6- (sulfamoyl- amino)- hexan-2-yl)- benzofuran-2- carboxamide | (DMSO-d₆) δ (ppm): 10.50 (s, 1 H), 9.24 (d, J = 7.6 Hz, 1 H), 8.76 (dd, J = 4.1, 1.6 Hz, 1 H), 8.62 (d, J = 7.8 Hz, 1 H), 8.39 (dd, J = 8.2, 1.6 Hz, 1 H), 7.81 (d, J = 8.0 Hz, 1 H), 7.72-7.66 (m, 3 H), 7.59-7.56 (m, 2 H), 7.49 (t, J = 7.4 Hz, 1 H), 7.35 | |
| # (t, J = 7.6 Hz, 1 H), 6.47-6.44 (m, 3 H), 4.78-4.72 (m, 1 H), 2.87 (q, J = 6.3 Hz, 2 H), 2.02-1.88 (m, 2 H), 1.60-1.40 (m, 4 H). LRMS: (calc.) 495.2 (found) 496.2 (MH)+ | M, C, A, J, D, B | | | | | |
| 23 | 52 | 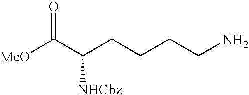 | 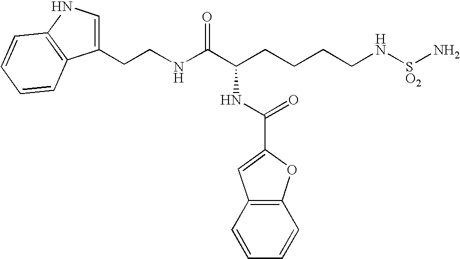 | (S)-N-(1-(2- (1H-indol-3- yl)ethylamino)- 1-oxo-6- (sulfamoyl- amino)- hexan-2-yl)- benzofuran-2- carboxamide | (DMSO-d₆) δ (ppm): 10.78 (s, 1 H), 8.51 (d, J = 8.2 Hz, 1 H), 8.16 (t, J = 5.7 Hz, 1 H), 7.77 (d, J = 7.0 Hz, 1 H), 7.67 (d, J = 8.2 Hz, 1 H), 7.62 (s, 1 H), 7.52 (d, J = 8.0 Hz, 1 H), 7.46 (t, J = 7.0 Hz, 1 H), 7.34 (d, J = 7.6 Hz, 1 H), | |
| # 7.30 (d, J = 7.0 Hz, 1 H), 7.13 (d, J = 1.8 Hz, 1 H), 7.03 (t, J = 7.0 Hz, 1 H), 6.94 (t, J = 7.8 Hz, 1 H), 6.43-6.41 (m, 3 H), 4.43-4.37 (m, 1 H), 2.84-2.79 (m, 4 H), 1.80-1.64 (m, 2 H), 1.53-1.22 (m, 6 H). LRMS: (calc.) 511.2 (found) 512.2 (MH)+ | M, C, A, J, D, B | | | | | |
| 24 | 53 | 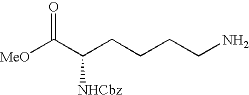 | 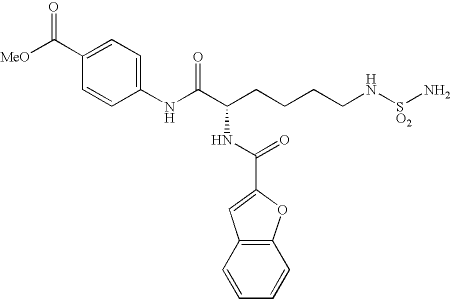 | (S)-N-(1-(3,4- dimethoxy- phenylamino)- 1-oxo-6- (sulfamoyl- amino)- hexan-2-yl)- benzofuran-2- carboxamide | (DMSO-d₆) δ (ppm): 10.50 (s, 1 H), 8.83 (d, J = 7.6 Hz, 1 H), 7.91 (d, J = 8.8 Hz, 2 H), 7.79-7.74 (m, 3 H), 7.68 (d, J = 8.6 Hz, 1 H), 7.64 (s, 1 H), 7.47 (t, J = 8.4 Hz, 1 H), 7.33 (t, J = 7.6 Hz, 1 H), 6.47-6.44 (m, 3 H), 4.58 (q, J = 6.8 Hz, 1 | |
| # H), 3.80 (s, 3 H), 2.85 (q, J = 6.3 Hz, 2 H), 1.87-1.78 (m, 2 H), 1.58-1.32 (m, 4 H). LRMS: (calc.) 502.2 (found) 503.2 (MH)+ | M, C, A, J, D, B | | | | | |
| 25 | 54 | 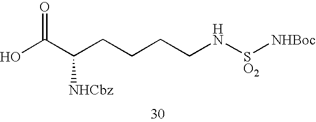 | 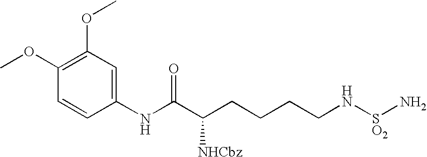 | (S)-benzyl 1- (3,4- dimethoxy- phenylamino)- 1-oxo-6- (sulfamoyl- amino)- hexan-2- ylcarbamate | (DMSO-d₆) δ (ppm) ¹H: 9.85 (s, 1 H), 7.50 (d, J = 7.6 Hz, 1 H), 7.35-7.20 (m, 6 H), 7.10 (dd, J = 8.6, 2.2 Hz, 1 H), 6.86 (d, J = 8.8 Hz, 1 H), 6.44 (s, 2 H), 6.42 (t, J = 6.1 Hz, 1 H), 5.01 (s, 2 H), 4.06 (q, J = 5.3 Hz, 1 H), 3.70 (s, 3 H), 3.69 (s, 3 H), | |
| # 2.82 (q, J = 6.5 Hz, 2 H), 1.68-1.52 (m, 2 H), 1.52-1.20 (m, 6 H). LRMS (ESI): (calc.) 494.2 (found) 495.3 (MH)+ | D, B | | | | | |
| 26 | 55 | 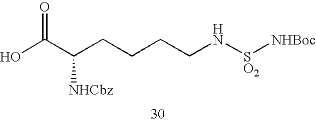 | 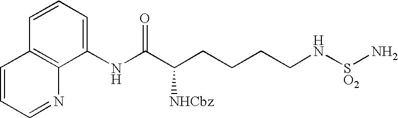 | (S)-benzyl 1- oxo-1- (quinolin-8- ylamino)-6- (sulfamoyl- amino)- hexan-2- ylcarbamate | (DMSO-d₆) δ (ppm): 10.46 (s, 1 H), 8.84 (dd, J = 4.3, 1.6 Hz, 1 H), 8.63 (dd, J = 7.6, 1.2 Hz, 1 H), 8.42 (dd, J = 8.2, 1.6 Hz, 1 H), 8.10 (d, J = 7.4 Hz, 1 H), 7.69-7.63 (m, 2 H), 7.58 (t, J = 7.8 Hz, 1 H), 7.37-7.35 (m, 2 H), 7.29-7.27 (m, 2 H), 6.44-6.41 (m, 3 H), 5.15-5.02 | |
| # (m, 2 H), 4.24-4.21 (m, 1 H), 2.84 (q, J = 6.5 Hz, 2 H), 1.91-1.77 (m, 1 H), 1.76-1.61 (m, 1 H), 1.58-1.38 (m, 4 H). LRMS: (calc.) (calc.) 485.2 (found) 486.2 (MH)+ | D, B | | | | | |
| 27 | 56 | 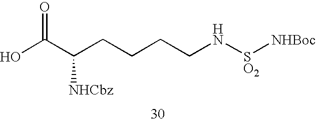 | 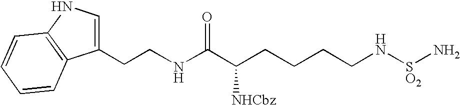 | (S)-benzyl 1- (2-(1H-indol- 3-yl)ethyl- amino)-1- oxo-6- (sulfamoyl- amino)- hexan-2- ylcarbamate | (DMSO-d₆) δ (ppm): 10.78 (s, 1 H), 7.97 (t, J = 5.3 Hz, 1 H), 7.52 (d, J = 7.8 Hz, 1 H), 7.35-7.27 (m, 7 H), 7.12 (d, J = 2.2 Hz, 1 H), 7.04 (td, J = 7.2, 1.4 Hz, 1 H), 6.95 (td, J = 8.0, 1.0 Hz, 1 H), 6.44 (s, 2 H), 6.41 (d, J = 6.1 Hz, 1 H), 5.05-4.97 (m, | |
| # 2 H), 3.90 (q, J = 4.7 Hz, 1 H), 3.38-3.23 (m, 2 H), 2.83-2.77 (m, 4 H), 1.62-1.39 (m, 4 H), 1.38-1.19 (m, 2 H). LRMS: (calc.) 501.2 (found) 502.3 (MH)+ | D, B | | | | | |
| 28 | 57 | 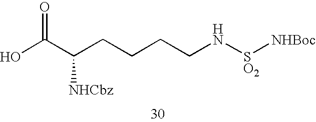 | 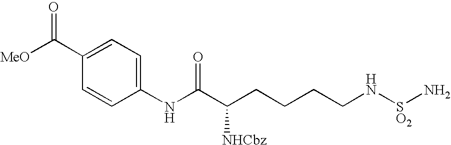 | (S)-methyl 4- (2-(benzyl- oxycarbonyl amino)-6- (sulfamoyl- amino)- hexanamido)- benzoate | (DMSO-d₆) δ (ppm): 10.38 (s, 1 H), 7.90 (d, J = 8.6 Hz, 2 H), 7.73 (d, J = 8.8 Hz, 2 H), 7.62 (d, J = 7.6 Hz, 1 H), 7.37-7.28 (m, 4 H), 7.24-7.10 (m, 1 H), 6.43 (s, 2 H), 6.43 (t, J = 6.3 Hz, 1 H), 5.01 (s, 2 H), 4.11 (q, J = 5.3 Hz, 1 H), 3.80 (s, 3 H), 2.83 (q, J = 6.7 | |
| # Hz, 2 H), 1.72-1.53 (m, 2 H), 1.53-1.23 (m, 4 H). LRMS: (calc.) 492.2 (found) 493.2 (MH)+ | D, B | | | | | |
| 29 | 58 | 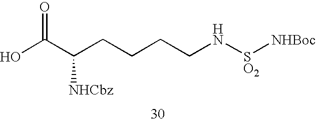 | 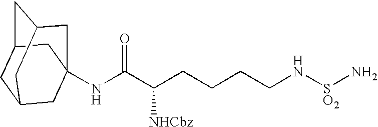 | (S)-benzyl 1- (adamantanyl- amino)-1-oxo- 6-(sulfamoyl- amino)- hexan-2- ylcarbamate | (DMSO-d₆) δ (ppm): 7.37-7.29 (m, 5 H), 7.23 (s, 1 H), 7.18 (d, J = 8.2 Hz, 1 H), 6.43 (s, 2 H), 6.40 (t, J = 6.3 Hz, 1 H), 5.00 (s, 2 H), 3.88 (q, J = 5.5 Hz, 1 H), 2.79 (q, J = 6.5 Hz, 2 H), 2.00-1.93 (m, 3 H), 1.93-1.78 (m, 6 H), 1.63-1.20 (m, 12 H). LRMS: (calc.) 492.2 (found) | |
| # 493.3 (MH)+ | D, B | | | | | |
| 30 | 59 | 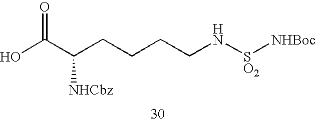 | 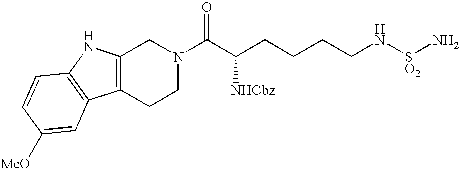 | (S)-benzyl 1- (6-methoxy- 3,4-dihydro- 1H-pyrido[3,4- b]indol- 2(9H)-yl)-1- oxo-6- (sulfamoyl- amino)- hexan-2- ylcarbamate | (DMSO-d₆) δ (ppm): 10.66 (s, 1 H), 7.57 (d, J = 8.2 Hz, 1 H),, 7.53-7.29 (m, 4 H), 7.20-7.10 (m, 2 H), 6.86 (d, J = 2.2 Hz, 1 H), 6.66 (dd, J = 8.6, 2.3 Hz, 1 H), 6.44-6.42 (m, 3 H), 4.99 (s, 2 H), 4.80-4.47 (m, 3 H), 3.91-3.72 (m, 2 H), | |
| # 3.73 (s, 3 H), 2.83 (q, J = 6.5 Hz, 2 H), 2.78-2.59 (m, 2 H), 1.62-1.23 (m, 6 H). LRMS: (calc.) 543.2 (found) 544.3 (MH)+ | D, B | | | | | |
| 31 | 60 | 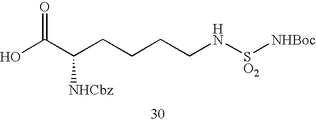 | 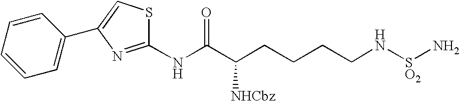 | (S)-benzyl 1- oxo-1-(4- phenylthiazol- 2-ylamino)-6- (sulfamoyl- amino)- hexan-2- ylcarbamate | (DMSO-d₆) δ (ppm): 12.39 (s, 1 H), 7.88 (d, J = 7.2 Hz, 2 H), 7.69 (d, J = 7.4 Hz, 1 H), 7.62 (s, 1 H), 7.42 (t, J = 7.4 Hz, 2 H), 7.38-7.28 (m, 5 H), 7.17-7.12 (m, 1 H), 6.44 (s, 2 H0, 6.43 (t, J = 6.1 Hz, 1 H), 5.02 (s, 2 H), 4.25 (q, J = 5.3 Hz, 1 H), 2.82 (q, J = | |
| # 6.3 Hz, 2 H), 1.78-1.59 (m, 2 H), 1.52-1.24 (m, 4 H). LRMS: (calc.) 517.1 (found) 518.2 (MH)+ | D, B | | | | | |
| 32 | 61 | 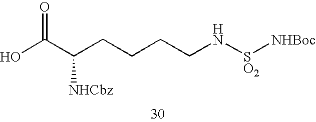 | 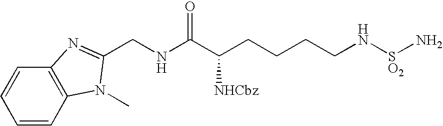 | (S)-benzyl 1- ((1- methyl-1H- benzo[d]- imidazol-2-yl)- methylamino)- 1-oxo-6- (sulfamoyl- amino)- hexan-2- ylcarbamate | (DMSO-d₆) δ (ppm): 8.71 (s, 1 H), 7.72 (d, J = 8.4 Hz, 1 H), 7.69 (d, J = 7.8 Hz, 1 H), 7.51 (d, J = 7.8 Hz, 1 H), 7.44-7.35 (m, 2 H), 7.33-7.23 (m, 5 H), 6.44-6.40 (m, 3 H), 5.00 (s, 2 H), 4.72-4.61 (m, 2 H), 3.98 (q, J = 4.9 Hz, 1 H), | |
| # 3.83 (s, 3 H), 2.80 (q, J = 6.1 Hz, 2 H), 1.69-1.59 (m, 1 H), 1.59-1.48 (m, 1 H), 1.48-1.20 (m, 4 H). LRMS: (calc.) 502.2 (found) 503.3 (MH)+ | D, B | | | | | |
| 33 | 62 | 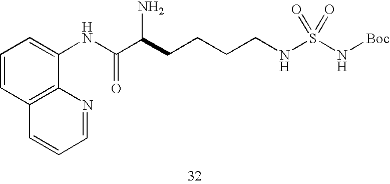 | 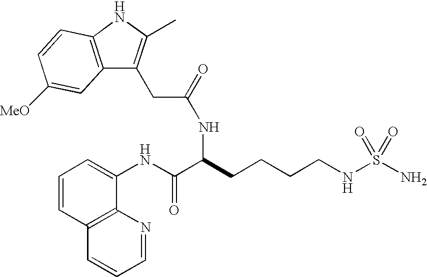 | (S)-2-(2-(5- methoxy-2- methyl- 1H-indol-3- yl)acetamido)- N-(quinolin-8- yl)-6- (sulfamoyl- amino)- hexanamide | (MeOD-d₄) δ (ppm): 8.76 (d, J = 4.4 Hz, 1 H), 8.58 (d, J =8.0 Hz, 1 H), 8.26 (d, J = 8.4 Hz, 1 H), 7.60 (d, J = 8.0 Hz, 1 H), 7.52 (d, J = 7.6 Hz, 1 H), 7.50 (d, J = 8.4 Hz, 1 H), 7.11 (d, J = 8.8 Hz, 1 H), 6.96 (d, J = 2.4 Hz, 1 H), | |
| # 6.63 (dd, J =2.4, 8.8 Hz, 1 H), 4.62 (q, J =4.8 Hz, 1 H), 3.81-3.67 (m, 2 H), 3.65 (s, 3 H), 2.95 (t, J =7.2 Hz, 2 H), 2.42 (s, 3 H), 2.01-1.96 (m, 1 H), 1.81-1.71 (m, 1 H), 1.58-1.47 (m, 2 H), 1.46-1.36 (m, 2 H). LRMS: 552.6 (calc) 553.4 (found) | A, B | | | | | |
| 34 | 63 | 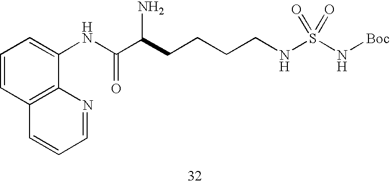 | 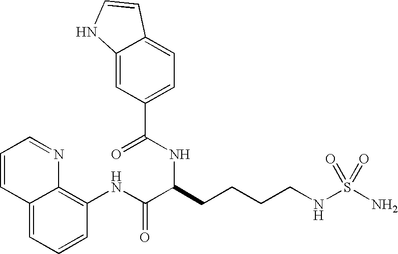 | (S)-N-(1-oxo- 1-(quinolin-8- ylamino)-6- (sulfamoyl- amino)- hexan-2-yl)- 1H-indole-6- carboxamide | (CD₃CN) δ (ppm) 1 H: 10.61 (br, 1 H), 9.70 (br, 1 H), 8.74-8.72 (m, 2 H), 8.29 (d, 1 H, J =8.4 Hz), 8.12 (s, 1 H), 7.69 (q, 2 H, J = 8.0 Hz), 7.62 (q, 2 H, J = 6.4 Hz), 7.56-7.51 (m, 3 H), 7.47 (t, 1 H, J = 2.8 Hz), 6.59 (s, 1 H), 5.16 (br, 2 H), 5.07 (br, 1 H), | |
| # 4.83-4.77 (m, 1 H), 3.06-3.02 (m, 2 H), 2.18-2.12 (m, 2 H), 1.69-1.56 (m, 4 H). LRMS (ESI): (calc.) 494.6 (found) 495.5 (MH)+ | A, B | | | | | |

| | | | | | | Prep- |
|---|---|---|---|---|---|---|
| | | | | | | ara- |
| | | | | | | tive |
| | | | | | | se- |
| Ex | Cpd | Starting Material | Structure | Name | Characterization | quence |
| 40 | 64 | 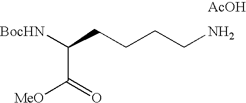 | 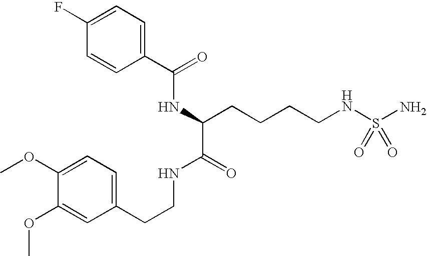 | (S)-N-(1- (3,4-dimeth- oxyphen- ethylamino)- 1-oxo-6- (sulfamoyl- amino)- hexan-2-yl)- 4-fluoro- benzamide | (MeOD-d₄) d (ppm) 1 H: 7.89 (m, 2 H), 7.19 (t, J = 8.8 Hz, 2 H), 6.79 (m, 3 H), 4.42 (dd, J = 6.0, 8.8 Hz, 1 H), 3.80 (s, 3 H), 3.75 (s, 3 H), 3.49 (m, 1 H), 3.38 (m, 1 H), 2.99 (t, J = 7.2 Hz, 2 H) 2.75 (t, J = 6.8 Hz, 2 H)., 1.80-1.70 | |
| # (m, 2 H), 1.59-1.52 (m, 2 H), 1.46-1.28 (m, 2 H) LRMS (ESI): (calc.) 510.2 (found) 509.1 (MH)− | O, E, I, J, A, C | | | | | |
| 41 | 65 | 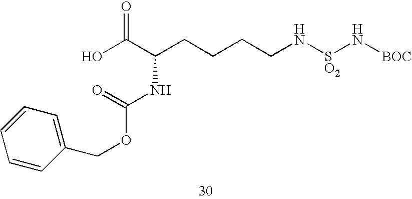 | 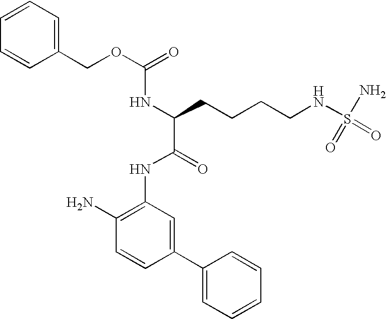 | (S)-benzyl 1- (4-amino- biphenyl-3- ylamino)-1- oxo-6- (sulfamoyl- amino) hexan-2- ylcarbamate | (CD₃OD) d (ppm) 1 H: 7.52 (d, J = 7.6 Hz, 2 H), 7.38-7.22 (m, 10 H), 6.90 (d, J = 8.4 Hz, 1 H), 5.12 (s, 2 H), 4.24 (dd, J = 4.0, 8.4 Hz, 1 H), 3.05 (t, J =6.4 Hz, 2 H), 1.91 (m, 1 H), 1.80 (m, 1 H), 1.65-1.52 (m, 4 H) LRMS (ESI): (calc.) 525.6 (found) | |
| #526.4 (MH)+ | D, E | | | | | |
| 42 | 66 | 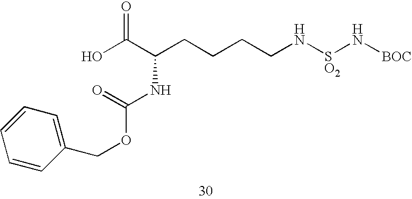 | 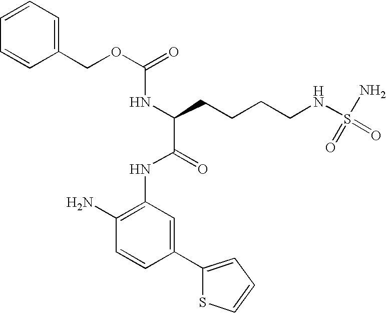 | (S)-benzyl 1- (2-amino-5- (thiophen-2- yl)phenyl- amino)-1- oxo-6-(sulf- amoylamino) hexan-2- ylcarbamate | (MeOD-d₄) d (ppm) 1 H: 7.39-7.17 (m, 9 H), 7.01 (t, J = 3.6 Hz, 1 H), 6.83 (d, J = 8.4 Hz, 1 H), 5.12 (s, 2 H), 4.23 (dd, J = 6.4, 8.8 Hz, 1 H), 3.05 (t, J =6.0 Hz, 2 H), 1.91 (m, 1 H), 1.79 (m, 1 H), 1.63-1.51 (m, 4 H) LRMS (ESI): (calc.) 531.6 | |
| # (found) 532.4 (MH)+ | D, E | | | | | |
| 43 | 67 | 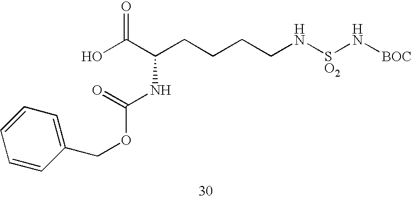 | 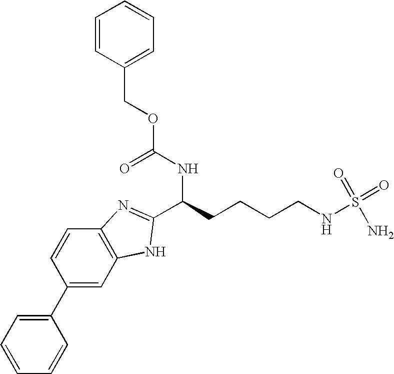 | (S)-benzyl 1- (5-phenyl- 1H-benzo- [d]imidazol- 2-yl)-5-(sulf- amoylamino) pentyl- carbamate | (CD₃OD) d (ppm) 1 H: 7.73-7.31 (m, 13 H), 5.11 (d, J = 12.8 Hz, 1 H), 5.07 (d, J = 12.4 Hz, 1 H), 4.93 (m, 1 H), 3.02 (t, J = 6.4 Hz, 2 H), 2.10 (m, 1 H), 1.96 (m, 1 H), 1.63-1.47 (m, 4 H) LRMS (ESI): (calc.) 507.6 (found) 508.4 (MH)+ | D, Q, E |
| 44 | 68 | 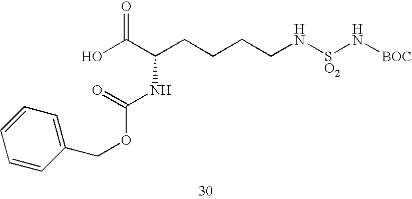 | 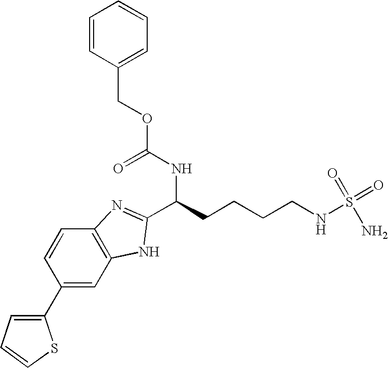 | (S)-benzyl 5- (sulfamoyl- amino)-1-(5- (thiophen-2- yl)-1H- benzo[d]- imidazol-2- yl)pentyl- carbamate | (MeOD-d₄) d (ppm) 1 H: 7.76 (s, 1 H), 7.53 (s, 2 H), 7.37-7.07 (m, 8 H), 5.14 (d, J = 11.6 Hz, 1 H), 5.08 (d, J = 13.2 Hz, 1 H), 4.93 (m, 1 H), 3.03 (t, J = 6.8 Hz, 2 H), 2.08 (m, 1 H), 1.96 (m, 1 H), 1.63-1.47 (m, 4 H) LRMS (ESI): (calc.) | |
| # 513.6 (found) 514.4 (MH)+ | D, Q, E | | | | | |
| 45 | 69 | 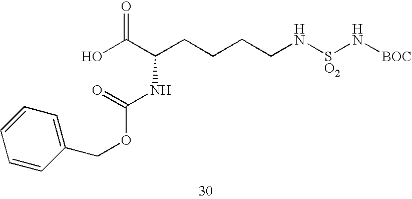 | 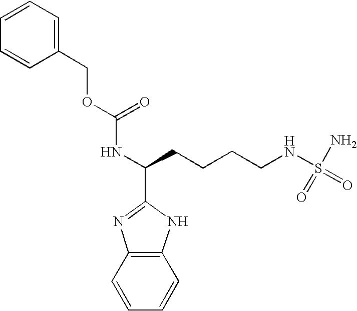 | (S)-benzyl 1- (1H-benzo- [d]imidazol- 2-yl)-5-(sulf- amoylamino) pentyl- carbamate | (CD₃OD) d (ppm) 1 H: 8.19 (s, 1 H), 7.52 (m, 2 H), 7.38-7.21 (m, 6 H), 7.01 (bs, 1 H), 5.13 (d, J = 10.0 Hz, 1 H), 5.07 (d, J =12.8 Hz, 1 H), 4.93 (t, J = 7.6 Hz, 1 H), 3.02 (t, J = 6.8 Hz, 2 H), 2.08 (m, 1 H), 1.95 (m, 1 H), 1.62-1.43 (m, 4 H) LRMS (ESI): | |
| # (calc.) 431.1 (found) 432.3 (MH)+ | D, Q, E | | | | | |
| 46 | 70 | 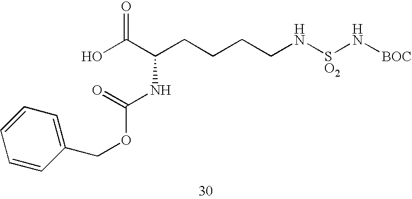 | 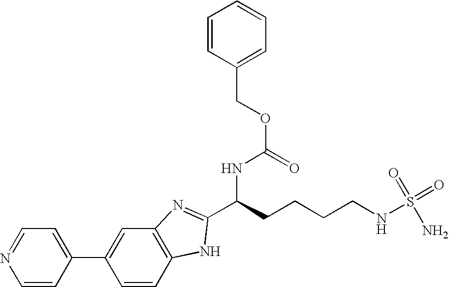 | (S)-benzyl 1- (5-(pyridin- 4-yl)-1H- benzo[d]- imidazol- 2-yl)-5-(sulf- amoylamino) pentyl- carbamate | (MeOD-d₄) d (ppm) 1 H: 8.56 (d, J =5.6 Hz, 2 H), 8.14 (m, 1 H), 7.93 (s, 1 H), 7.77 (d, J = 6.0 Hz, 2 H), 7.66 (s, 2 H), 7.35 (m, 4 H), 5.14 (d, J = 12.0 Hz, 1 H), 5.07 (d, J = 12.4 Hz, 2 H), 4.96 (m, 1 H), 3.03 (t, J = 6.8 Hz, 2 | |
| # H), 2.15-1.93 (m, 2 H), 1.63-1.48 (m, 4 H) LRMS (ESI): (calc.) 508.1 (found) 509.3 (MH)+ | D, Q, E | | | | | |
| 47 | 71 | 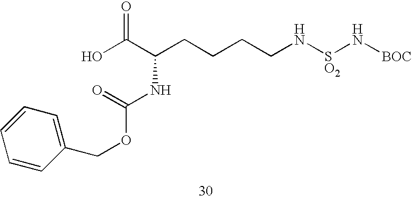 | 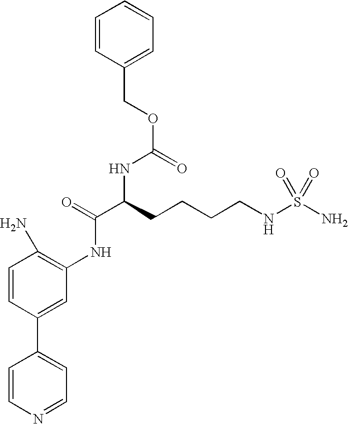 | (S)-benzyl 1- (2-amino-5- (pyridin-4- yl)phenyl- amino)-1- oxo-6-(sulf- amoylamino) hexan-2- ylcarbamate | (MeOD-d₄) d (ppm) 1 H: 8.46 (m, 2 H), 8.21 (m, 1 H), 7.68 (d, J = 6 Hz, 2 H), 7.53 (m, 2 H), 7.39-7.28 (m, 4 H), 6.92 (d, J = 8.4 Hz, 1 H), 5.12 (s, 2 H), 4.22 (t, J = 6.0 Hz, 1 H), 3.06 (t, J =6.0 Hz, 2 H), 1.93-1.54 (m, 6 H) LRMS (ESI): | |
| # (calc.) 526.2 (found) 527.3 (MH)+ | D, E | | | | | |
| 48 | 72 | 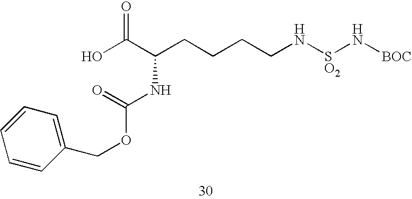 | 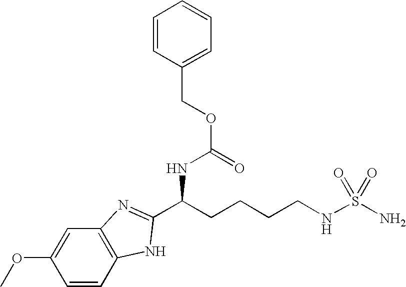 | (S)-benzyl 1- (5-methoxy- 1H-benzo- [d]imidazol- (sulfamoyl- amino) pentyl- carbamate | (MeOD-d₄) d (ppm) 1 H: 8.27 (m, 1 H), 7.33 (m, 5 H), 7.02 (m, 2 H), 6.85 (d, J = 8.4 Hz, 1 H), 5.13 (d, J = 12.8 Hz, 1 H), 5.07 (d, J = 12.8 Hz, 1 H), 4.93 (m, 1 H), 3.82 (s, 3 H), 3.01 (t, J =6.8 Hz, 2 H), 2.05-1.93 m, 2 H), 1.91-1.43 (m, 4 H). LRMS (ESI): | |
| # (calc.) 461.1 (found) 462.3 (MH)+ | D, Q, E | | | | | |
| 49 | 79 | 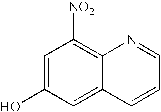 | 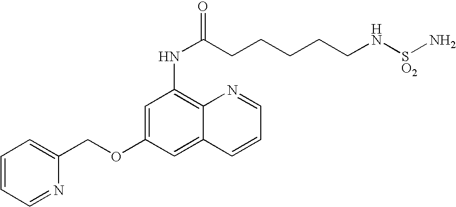 | N-(6- (pyridin-2- ylmethoxy)- quinolin-8- yl)-6-(sulfa- moylamino)- hexanamide | (DMSO-d₆) d (ppm): 10.04 (s, 1 H), 8.74 (dd, J =4.2, 1.5 Hz, 1 H), 8.59 (d, J = 4.3 HZ, 1 H), 8.43 (d, J =2.5 Hz, 1 H), 8.24 (8.2, 1.4 Hz, 1 H), 7.84 (td, J = 7.6, 1.6 Hz, 1 H), 7.58-7.54 (m, 2 H), 7.37-7.34 (m 1 H), 7.15 (d, J = 2.7 Hz, 1 H), 6.44 (s, 3 H), 5.29 | |
| # (s, 2 H), 2.87-2.82 (m, 2 H), 2.57 (t, J = 7.3 Hz, 2 H), 1.65-1.61 (m, 2 H), 1.51-1.48 (m, 2 H), 1.37-1.33 (m, 2 H). LRMS (ESI): (calc.) 443.2 (found) 444.3 (MH)+ | II, QQ, A, E, M, B | | | | | |
| 50 | 80 | 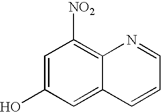 | 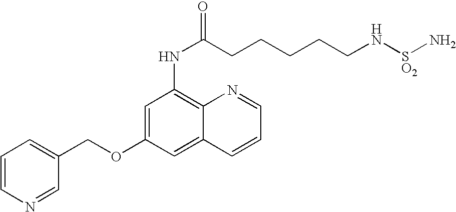 | N-(6- (pyridin-3- ylmethoxy)- quinolin-8- yl)-6-(sulfa- moylamino)- hexanamide | (MeOD-d₄) d (ppm): 8.72 (dd, J =4.3, 1.6 Hz, 1 H), 8.56 (dd, J = 4.7, 1.4 Hz, 2 H), 8.49 (d, J = 2.7 Hz, 1 H), 8.18 (dd, J = 8.2, 1.4 Hz, 1 H), 7.58 (d, J = 6.1 Hz, 2 H), 7.50 (q, J = 4.2, 1 H), 7.06 (d, J = 2.7 Hz, 1 H), 5.34 (s, 2 H), 3.05 (t, J = 6.9 | |
| #Hz, 2 H), 2.63 (t, J = 7.4 Hz, 2 H), 1.84-1.80 (m, 2 H), 1.67-1.63 (m, 2 H), 1.53-1.52 (m, 2 H). LRMS (ESI): (calc.) 443.5 (found) 444.3 (MH)+ | II, QQ, A, E, M, B | | | | | |
| 51 | 81 | 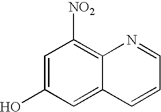 | 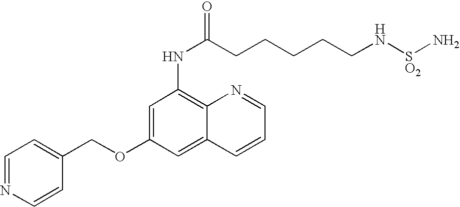 | N-(6- (pyridin-4- ylmethoxy)- quinolin-8- yl)-6-(sulfa- moylamino)- hexanamide | (MeOD-d₄) d (ppm): 8.72-8.70 (m, 2 H), 8.52 (d, J = 3.7 Hz, 1 H), 8.45 (d, J = 2.5 Hz, 1 H), 8.20 (dd, J =8.3, 1.5 Hz, 1 H), 8.02 (d, J = 7.8 Hz, 1 H), 7.52-7.49 (m, 2 H), 7.13 (d, J =2.7 Hz, 1 H), 5.31 (s, 2 H), 3.05 (t, J =6.9 Hz, 2 H), 2.62 (t, J = 7.4 Hz, | |
| # 2 H), 1.83-1.79 (m, 2 H), 1.66-1.63 (m, 2 H), 1.53-1.51 (m, 2 H). LRMS (ESI): (calc.) 443.5 (found) 444.4 (MH)+ | II, QQ, A, E, M, B | | | | | |
| 52 | 84 | 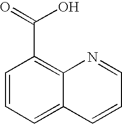 | 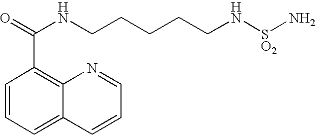 | N-(5-(sulfa- moylamino)- pentyl)-quinoline-8- carboxamide | (MeOD-d₄) d (ppm): 8.98 (dd, J =4.3 Hz, 1.8 Hz, 1 H), 8.63 (dd, J = 7.4 Hz, 1.6 Hz, 1 H), 8.43 (dd, J = 8.4 Hz, 1.8 Hz, 1 H), 8.10-8.08 (m, 1 H), 7.69 (t, J = 7.4 Hz, 1 H), 7.60-7.57 (m, 1 H), 3.58-3.54 (m, 2 H), 3.09-3.05 (m, 2 H), 1.81-1.56 (m, 6 H). LRMS (ESI): (calc.) 336.4 (found) 337.3 | |
| # (MH)+ | A, E, F | | | | | |
| 53 | 90 | 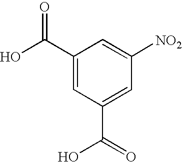 | 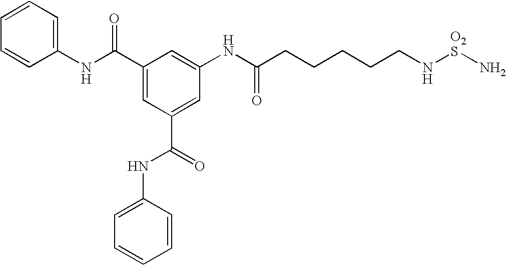 | N¹,N³- diphenyl-5- (6-(sulfa- moylamino) hexana- mido)iso- phthalamide | (MeOD-d₄) d (ppm) 1 H: 8.26 (d, J = 1.6 Hz, 2 H), 8.16 (t, J = 1.6 Hz, 1 H), 7.71-7.68 (m, 4 H), 7.36 (t, J =8.2 Hz, 4 H), 7.17-7.12 (m, 2 H), 3.04 (t, J = 7.0 Hz, 2 H), 2.44 (t, J =7.4 Hz, 2 H), 1.74 (quintet, J = 7.6 Hz, 2 H), 1.61 (quintet, J = 7.2 | |
| # Hz, 2 H), 1.50-1.42 (m, 2 H). LRMS (ESI): (calc.) 523.2 (found) 524.2 (MH)+ | A, C, A, B | | | | | |
| 203 | 206 | 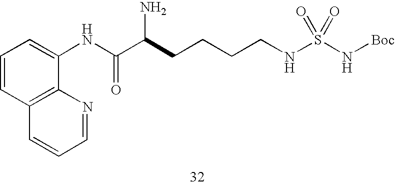 | 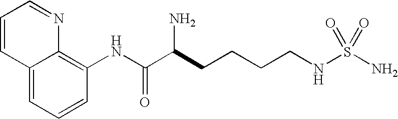 | (S)-2-amino- N-(quinolin- 8-yl)-6- (sulfamoyl- amino)- hexanamide | (CD₃OD) δ (ppm): 8.92 (dd, 1 H, J =1.6, 4.0 Hz), 8.63 (d, 1 H, J = 7.6 Hz), 8.34 (d, 1 H, J =8.0 Hz), 7.70 (d, 1 H, J = 8.4 Hz), 7.60-7.56 (m, 2 H), 4.39 (t, 1 H, J =6.8 Hz), 3.05 (t, 2 H, J = 6.4 Hz), 2.11-2.02 (m, 2 H), 1.68-1.60 (m, 4 H). LRMS (ESI): (calc.) 351.4 | |
| # (found) 352.3 (MH)+ | B | | | | | |
| 204 | 207 | 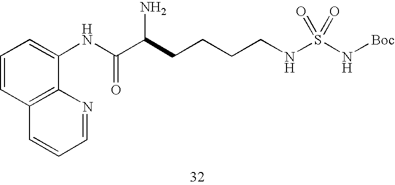 | 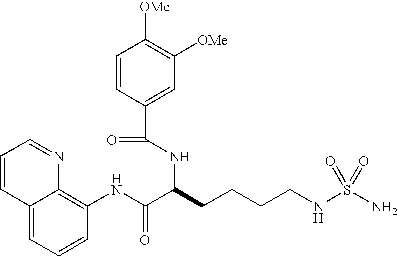 | (S)-3,4- dimethoxy- N-(1-oxo-1- (quinolin-8- ylamino)-6- (sulfamoyl- amino)hex- an-2-yl)- benzamide | (CD₃OD) δ (ppm): 8.83 (d, 1 H, J =4.4 Hz), 8.55 (d, 1 H, J = 7.6 Hz), 8.47 (d, 1 H, J = 8.4 Hz), 7.75 (d, 1 H, J = 7.6 Hz), 7.69-7.62 (m, 3 H), 7.58 (s, 1 H), 7.08 (d, 1 H, J = 8.4 Hz), 4.79 (q, 1 H, J = 5.2 Hz), 3.91 (s, 6 H), 3.08 | |
| # (t, 2 H, J = 6.4 Hz), 2.23-2.12 (m, 1 H), 2.07-1.96 (m, 1 H), 1.71-1.61 (m, 4 H). LRMS (ESI): (calc.) 515.2 (found) 516.4 (MH)+ | D, B | | | | | |
| 206 | 209 | 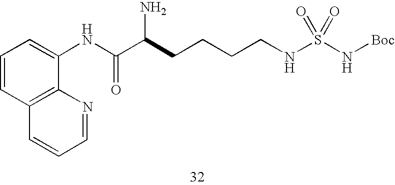 | 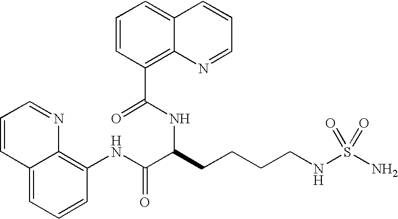 | (S)-N-(1- oxo-1- (quinolin-8- ylamino)-6- (sulfamoyl- amino)hex- an-2-yl)- quinoline-8- carboxamide | (CD₃OD) δ (ppm): 1 H: 9.11 (d, 1 H, J = 4.0 Hz), 8.68 (dd, 2 H, J = 7.6, 11.6 Hz), 8.52 (d, 1 H, J = 8.4 Hz), 8.48 (d, 1 H, J =4.0 Hz), 8.24 (d, 1 H, J = 8.4 Hz), 8.20 (d, 1 H, J = 8.0 Hz), 7.76 (t, 1 H, J = 7.6 Hz), 7.65 (q, 1 | |
| # H, J = 4.4 Hz), 7.61 (d, 1 H, J = 8.0 Hz), 7.57 (t, 1 H, J = 7.6 Hz), 7.44 (q, 1 H, J = 4.0 Hz), 5.02 (q, 1 H, J = 5.2 Hz), 3.08 (t, 2 H, J = 6.4 Hz), 2.24-2.12 (m, 2 H), 1.78-1.72 (m, 4 H). LRMS (ESI): (calc.) 506.2 (found) 507.4 (MH)+ | D, B | | | | | |
| 207 | 210 | 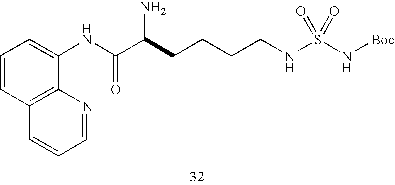 | 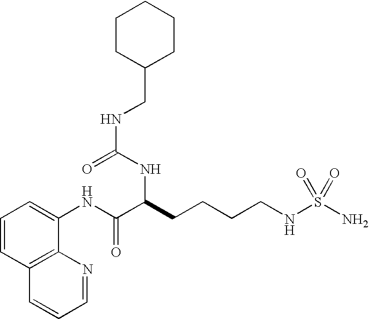 | (S)-2-(3- (cyclohexyl- methyl) ureido)-N- (quinolin-8- yl)-6-(sulfa- moylamino)- hexanamide | (CD3OD) δ (ppm) 1 H: 8.81 (s, 1 H), 8.66 (d, 1 H, J = 7.6 Hz), 8.29 (d, 1 H, J = 8.4 Hz), 7.62 (d, 1 H, J =8.0 Hz), 7.56-7.53 (m, 2 H), 4.40 (q, 1 H, J = 4.4 Hz), 3.07 (dd, 1 H, J = 6.8, 13.6 Hz), 3.05 (t, 2 H, J = 7.2 Hz), 2.94 (dd, 1 H, J = 6.4, | |
| #13.6 Hz), 2.03-1.99 (m, 1 H), 1.81-1.73 (m, 3 H), 1.69-1.52 (m, 7 H), 1.45-1.42 (m, 1 H), 1.28-1.15 (m, 3 H), 0.95-0.88 (m, 2 H). LRMS (ESI): (calc.) 490.2 (found) 491.5 (MH)+ | T, B | | | | | |
| 208 | 211 | 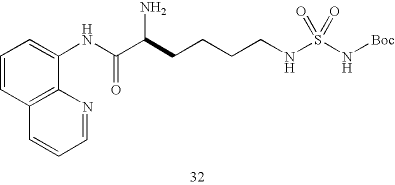 | 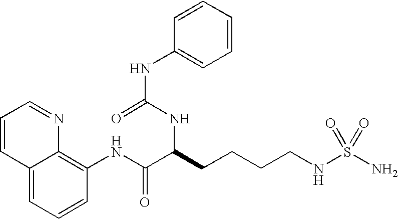 | (S)-2-(3- phenyl- ureido)-N- (quinolin-8- yl)-6-(sulfa- moylamino)- hexanamide | (DMSO-d6) δ(ppm) 1 H: 10.51 (s, 1 H), 8.86 (d, 1 H, J = 8.4 Hz), 8.74 (s, 1 H), 8.62 (d, 1 H, J = 8.0 Hz), 8.39 (d, 1 H, J = 8.0 Hz), 7.67 (d, 1 H, J =8.4 Hz), 7.62-7.56 (m, 2 H), 7.39 (d, 2 H, J = 7.6 Hz), 7.22 (t, 2 H, J = 7.2 Hz), 6.90 (t, 1 H, J = 7.6 Hz), | |
| # 6.79 (d, 1 H, J = 7.2 Hz), 6.46-6.43 (m, 3 H), 4.48-4.43 (m, 1 H), 2.85 (q, 2 H, J =6.4 Hz), 1.90-1.87 (m, 1 H), 1.72-1.67 (m, 1 H), 1.51-1.40 (m, 4 H). LRMS (ESI): (calc.) 470.2 (found) 471.4 (MH)+ | T, B | | | | | |
| 218 | 237 | 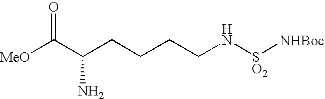 | 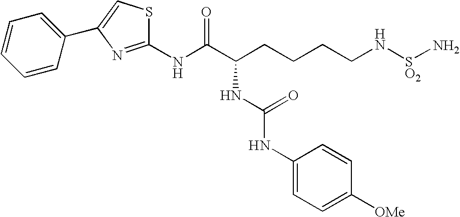 | (S)-2-(3-(4- methoxy- phenyl)- ureido)-N- (4-phenyl- thiazol-2-yl)- 6-(sulfa- moylamino)- hexanamide | (CD3CN) δ (ppm) 1 H: 10.49 (bs, 1 H), 7.91-7.88 (m, 2 H), 7.46-7.41 (m, 3 H), 7.36-7.31 (m, 4 H), 6.88-6.85 (m, 2 H), 5.81 (d, J = 7.2 Hz, 1 H), 5.17 (bs, 2 H), 5.09-5.05 (m, 1 H), 4.48-4.43 (m, 1 H), 3.75 (s, 3 H), 3.02 (q, J = 6.4 Hz, 2 H), 1.99-1.91 (m, | |
| # 1 H), 1.80-1.74 (m, 1 H), 1.64-1.47 (m, 4 H). LRMS (ESI): (calc.) 532.6 (found) 533.2 (MH)+ | T, J, U, B | | | | | |
| 219 | 238 | 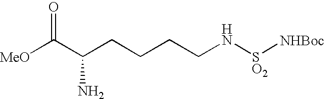 | 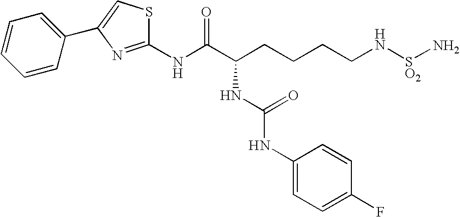 | (S)-2-(3-(4- fluoro- phenyl)- ureido)-N- (4-phenyl- thiazol-2-yl)- 6-(sulfa- moylamino)- hexanamide | (CD3CN) d (ppm) 1 H: 10.49 (bs, 1 H), 7.89-7.87 (m, 2 H), 7.60 (bs, 1 H), 7.45-7.41 (m, 4 H), 7.36-7.32 (m, 2 H), 7.04-7.00 (m, 2 H), 5.88 (d, J = 7.2 Hz, 1 H), 5.18 (bs, 2 H), 5.08-5.07 (m, 1 H), 4.51-4.46 (m, 1 H), 3.02 (q, J = 6.4 Hz, 2 H), 1.97-1.90 (m, 1 | |
| # H), 1.79-1.75 (m, 1 H), 1.62-1.46 (m, 4 H). LRMS (ESI): (calc.) 520.6 (found) 521.2 (MH)+ | T, J, U, B | | | | | |
| 220 | 239 | 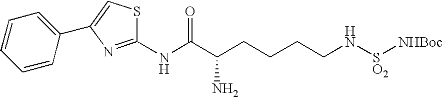 | 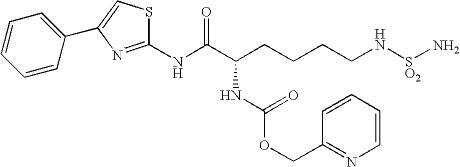 | (S)-pyridin- 2-ylmethyl 1-oxo-1-(4- phenyl- thiazol-2- ylamino)-6- (sulfamoyl- amino)hex- an-2-yl- carbamate | (CD3CN) d (ppm) 1 H: 8.61 (d, J = 4.8 Hz, 1 H), 7.91-7.89 (m, 2 H), 7.81-7.77 (m, 1 H), 7.47-7.41 (m, 3 H), 7.38-7.33 (m, 2 H), 7.30-7.27 (m, 1 H), 6.33 (d, J = 6.8 Hz, 1 H), 5.20 (s, 2 H), 5.18 (s, 2 H), 5.07 (bs, 1 H), 4.39-4.34 (m, 1 H), 3.02 (q, | |
| # J = 6.4 Hz, 2 H), 1.98-1.89 (m, 1 H), 1.80-1.74 (m, 1 H), 1.63-1.44 (m, 4 H). LRMS (ESI): (calc.) 518.6 (found) 519.2 (MH)+ | X, B | | | | | |
| 221 | 240 | 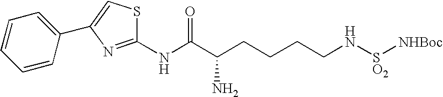 | 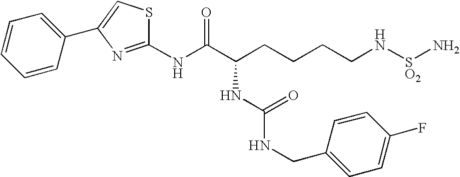 | (S)-2-(3-(4- fluoro- benzyl)- ureido)-N- (4-phenyl- thiazol-2- yl)-6-(sulfa- moylamino)-hexanamide | (CD3CN) d (ppm) 1 H: 7.91-7.88 (m, 2 H), 7.46-7.42 (m, 2 H), 7.37-7.30 (m, 1 H), 7.07-7.03 (m, 2 H), 5.76 (bs, 1 H), 5.66-5.63 (m, 1 H), 5.16 (bs, 2 H), 5.06 (bs, 1 H), 4.42-4.39 (m, 1 H), 4.33 (d, J = 5.6 Hz, 2 H), 4.33 (q, J = 6.4 Hz, 2 H), 1.98-1.85 (m, 1 H), | |
| # 1.79-1.68 (m, 1 H), 1.62-1.43 (m, 4 H). LRMS (ESI): (calc.) 534.63 (found) 535.2 (MH)+ | T, B | | | | | |
| 222 | 241 | 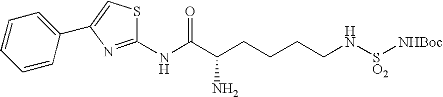 | 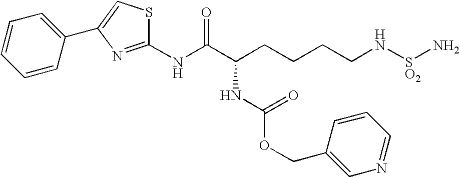 | (S)-pyridin- 3-ylmethyl 1-oxo-1-(4- phenyl- thiazol-2- ylamino)-6- (sulfa- moylamino)- hexan-2- ylcarbamate | (CD3CN) d (ppm) 1 H: 8.64-8.56 (m, 2 H), 7.92-7.89 (m, 2 H), 7.83-7.81 (m, 1 H), 7.47-7.34 (m, 6 H), 6.29-6.27 (m, 1 H), 5.22-5.08 (m, 5 H), 4.36-4.33 (m, 1 H), 3.02-2.98 (m, 1 H), 1.95-1.88 (m, 1 H), 1.80-1.71 (m, 1 H), 1.61-1.41 (m, 4 H). LRMS (ESI): (calc.) | |
| # 518.6 (found) 519.2 (MH)+ | X, B | | | | | |
| 223 | 242 | 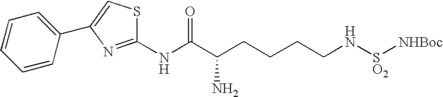 | 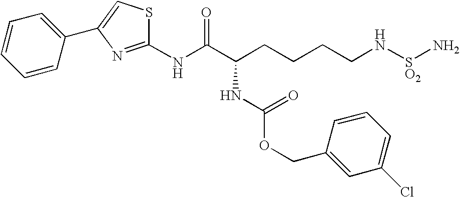 | (S)-3- chlorobenzyl 1-oxo-1-(4- phenyl- thiazol-2- ylamino)-6- (sulfa- moylamino)- hexan-2- ylcarbamate | (CD3CN) d (ppm) 1 H: 10.24 (bs, 1 H), 7.91-7.88 (m, 2 H), 7.47-7.40 (m, 3 H), 7.38-7.36 (m, 5 H), 6.23 (d, J = 7.6 Hz, 1 H), 5.14 (bs, 2 H), 5.11 (s, 2 H), 5.03 (bs, 1 H), 4.34-4.31 (m, 1 H), 3.01-3.00 (m, 2 H), 1.90-1.88 (m, 1 H), 1.79-1.72 (m, 1 H), | |
| # 1.63-1.50 (m, 4 H). LRMS (ESI): (calc.) 551.1 (found) 552.2 (MH)+ | X, B | | | | | |
| 224 | 243 | 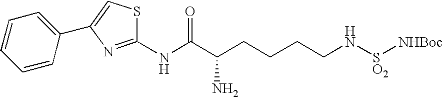 | 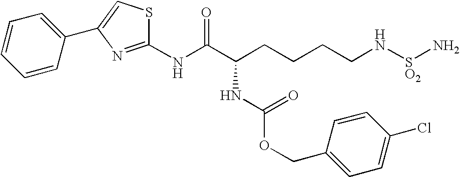 | (S)-4- chlorobenzyl 1-oxo-1-(4- phenyl- thiazol-2- ylamino)-6- (sulfa- moylamino)- hexan-2- ylcarbamate | (CD3CN) d (ppm) 1 H: 7.92-7.89 (m, 2 H), 7.47-7.43 (m, 3 H), 7.40-7.34 (m, 5 H), 6.16 (bs, 1 H), 5.13 (bs, 2 H), 5.11 (s, 2 H), 5.02 (bs, 1 H), 4.37-4.29 (m, 1 H), 3.01 (quartet, J = 6.4 Hz, 2 H), 1.94-1.88 (m, 1 H), 1.80-1.73 (m, 1 H), 1.61-1.56 (m, 2 H), | |
| #1.55-1.44 (m, 2 H). LRMS (ESI): (calc.) 551.1 (found) 552.2 (MH)+ | X, B | | | | | |
| 225 | 244 | 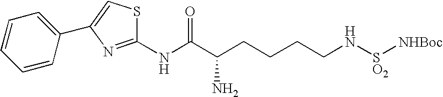 | 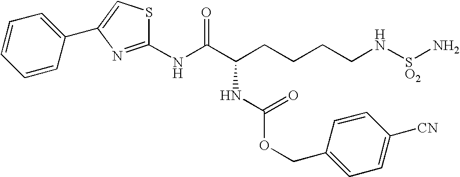 | (S)-4- cyanobenzyl 1-oxo-1-(4- phenyl- thiazol-2- ylamino)-6- (sulfa- moylamino)- hexan-2- ylcarbamate | (CD3CN) d (ppm) 1 H: 7.92-7.89 (m, 2 H), 7.75 (d, J = 8.0 Hz, 2 H), 7.55 (d, J = 7.6 Hz, 2 H), 7.47-7.43 (m, 2 H), 7.38-7.34 (m, 2 H), 6.26 (d, J = 7.6 Hz, 1 H), 5.20 (s, 2 H), 5.12 (bs, 2 H), 5.02 (bs, 1 H), 4.34-4.31 (m, 1 H), 3.01 (quartet, J = | |
| # 6.4 Hz, 2 H), 1.93-1.90 (m, 1 H), 1.80-1.73 (m, 1 H), 1.63-1.43 (m, 4 H). LRMS (ESI): (calc.) 542.1 (found) 543.2 (MH)+ | X, B | | | | | |
| 226 | 245 | 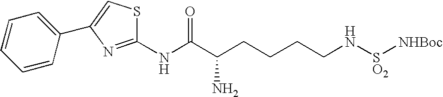 | 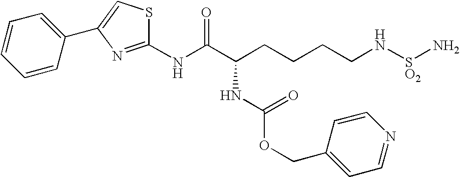 | (S)-pyridin- 4-ylmethyl 1-oxo-1-(4- phenyl- thiazol-2- ylamino)-6- (sulfa- moylamino)- hexan-2- ylcarbamate | (CD3CN) d (ppm) 1 H: 8.66-8.64 (m, 2 H), 7.90-7.88 (m, 2 H), 7.71-7.70 (m, 2 H), 7.46-7.42 (m, 2 H), 7.37-7.33 (m, 2 H), 5.25 (s, 2 H), 5.23-5.02 (m, 3 H), 4.39-4.35 (m, 1 H), 2.99 (t, J = 6.8 Hz, 2 H), 1.92-1.89 (m, 1 H), 1.82-1.75 (m, 1 H), 1.62-1.45 (m, | |
| #4 H). LRMS (ESI): (calc.) 518.6 (found) 519.2 (MH)+ | X, B | | | | | |
| 227 | 246 | 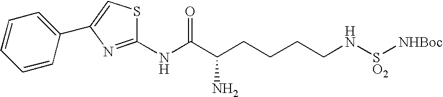 | 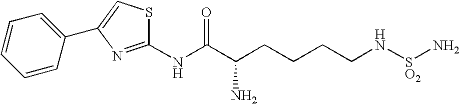 | (S)-2-amino- N-(4-phenyl- thiazol-2-yl)- 6-(sulfa- moylamino)- hexanamide | (CD3CN) d (ppm) 1 H: 7.93-7.91 (m, 2 H), 7.46-7.43 (m, 2 H), 7.37-7.34 (2 H), 5.12 (bs, 2 H), 5.02 (bs, 1 H), 3.56 (dd, J = 8.2, 4.6 Hz, 1 H), 3.03-3.00 (m, 2 H), 1.98-1.85 (m, 2 H), 3.65-1.48 (m, 6 H). LRMS (ESI): (calc.) 383.4 (found) 384.1 (MH)+ | B |
| 228 | 247 | 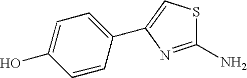 | 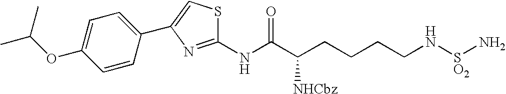 | (S)-benzyl 1-(4-(4-iso- propoxy- phenyl)- thiazol-2- ylamino)- 1-oxo-6- (sulfamoyl- amino)- hexan-2- ylcarbamate | (CD3CN) d (ppm) 1 H: 10.25 (bs, 1 H), 7.79 (d, J = 9.2 Hz, 2 H), 7.40-7.35 (m, 5 H), 7.19 (s, 1 H), 6.95 (d, J = 9.2 Hz, 2 H), 6.19 (d, J = 7.2 Hz, 1 H), 5.16-5.03 (m, 5 H), 4.65 (quintet, J = 4.5 Hz, 1 H), 4.34-4.31 (m, 1 H), 3.00 | |
| # (q, J = 6.8 Hz, 2 H), 1.94-1.86 (m, 1 H), 1.78-1.71 (m, 1 H), 1.60-1.40 (m, 4 H), 1.32 (d, J =6.4 Hz, 6 H). LRMS (ESI): (calc.) 575.7 (found) 576.3 (MH)+ | Z, U, B | | | | | |
| 229 | 248 | 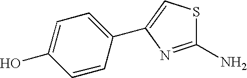 | 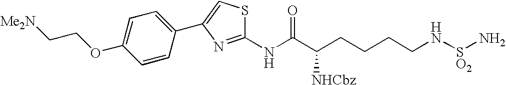 | (S)-benzyl 1-(4-(4-(2- (dimethyl- amino)- ethoxy)- phenyl)- thiazol-2- ylamino)- 1-oxo-6- (sulfamoyl- amino)- hexan-2- ylcarbamate | (CD3CN) d (ppm) 1 H: 8.33 (s, 1 H), 7.84-7.81 (m, 2 H), 7.40-7.34 (m, 5 H), 7.21 (s, 1 H), 7.10-6.97 (m, 2 H), 6.20 (d, J = 7.6 Hz, 1 H), 5.15-5.08 (m, 4 H), 4.34-4.32 (m, 1 H), 4.18 (t, J =5.6 Hz, 2 H), 2.99 (t, J = 6.8 | |
| # Hz, 2 H), 2.88 (t, J = 5.6 Hz, 2 H), 2.42 (s, 3 H), 1.81-1.70 (m, 2 H), 1.60-1.42 (m, 4 H). LRMS (ESI): (calc.) 604.7 (found) 605.3 (MH)+ | Z, U, B | | | | | |
| 324 | 269 | 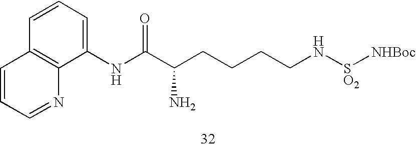 | 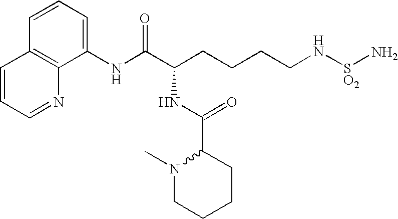 | 1-methyl-N- ((S)-1-oxo-1- (quinolin-8- ylamino)-6- (sulfamoyl- amino)- hexan-2-yl)- piperidine-2- carboxamide | (dmso-d6) d (ppm) 10.43 (s, 0.5 H), 10.37 (s, 0.5 H), 9.77 (bs, 1 H), 9.27 (bs, 1 H), 8.92-8.88 (m, 1 H), 8.63-8.60 (m, 1 H), 8.46-8.42 (m, 1 H), 7.76-7.65 (m, 2 H), 7.60 (t, J = 8.0 Hz, 1 H), 6.49-6.41 (m, 3 H), 4.66 (m, 1 H), 3.98-3.79 (m, 1 H), 3.40 (m, | |
| # 1 H), 3.20-3.03 (m, 1 H), 2.86 (q, J = 6.7 Hz, 1 H), 2.75 (bs, 3 H), 2.40 (m, 0.5), 2.14 (m, 0.5 H), 2.00-1.60 (m, 6 H), 1.60-1.37 (m, 5 H). TFA salt of a 1:1 mixture of two diasteromers. LRMS (ESI): (calc.) 476.2 (found) 477.3 (MH)+ | EE, B | | | | | |
| 236 | 270 | 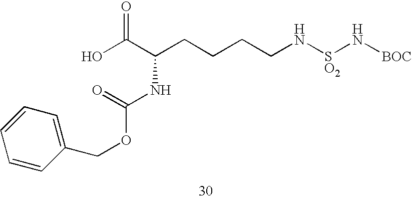 | 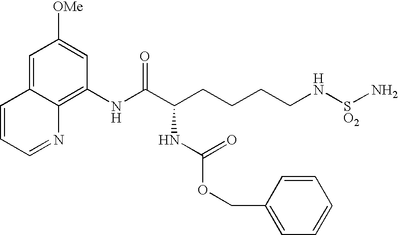 | (S)-benzyl 1- (6-methoxy- quinolin-8- ylamino)- 1-oxo-6- (sulfamoyl- amino)- hexan-2- ylcarbamate | (dmso-d6) d (ppm) 1 H: 10.42 (s, 1 H), 8.66 (dd, J = 4.3, 1.6 Hz, 1 H), 8.31-8.27 (m, 2 H), 8.10 (d ,J = 7.2 Hz, 1 H), 7.58 (dd, J =8.4, 4.3 Hz, 1 H), 7.39-7.32 (m, 2 H), 7.32-7.25 (m, 3 H), 7.09 (d, J = 2.7 Hz, 1 H), 6.44 (s, 2 H), 6.43 (t, J = 6.1 | |
| # Hz, 1 H), 5.14 (d, J = 13 Hz, 1 H), 5.03 (d, J = 13 Hz, 1 H), 4.25-4.18 (m, 1 H), 3.87 (s, 3 H), 2.84 (q, J = 6.7 Hz, 2 H), 1.90-1.78 (m, 1 H), 1.76-1.60 (m, 1 H), 1.57-1.36 (m, 4 H). LRMS (ESI): (calc.) 515.2 (found) 516.3 (MH)+ | D, B | | | | | |
| 237 | 271 | 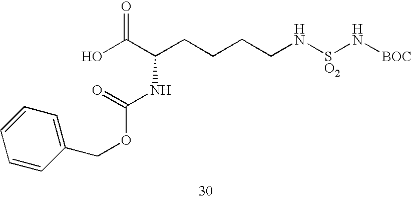 | 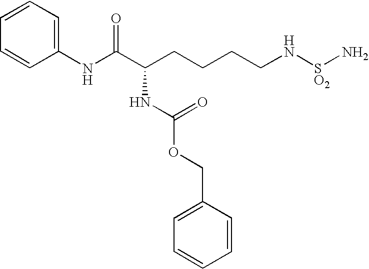 | (S)-benzyl 1-oxo-1- (phenyl- amino)-6- (sulfamoyl- amino)- hexan-2- ylcarbamate | (dmso-d6) d (ppm) 1 H: 10.00 (s, 1 H), 7.59-7.54 (m, 3 H), 7.35-7.15 (m, 7 H), 7.03 (t, J = 7.2 Hz, 1 H), 6.43 (s, 2 H), 6.42 (t, J = 6.1 Hz, 1 H), 5.01 (s, 2 H), 4.10 (q, J = 5.1 Hz, 1 H), 2.82 (q, J =6.3 Hz, 2 H), 1.71-1.52 (m, 2 H), 1.52-1.23 (m, 4 H). LRMS (ESI): | |
| # (calc.) 434.2 (found) 435.2 (MH)+ | D, B | | | | | |
| 238 | 272 | 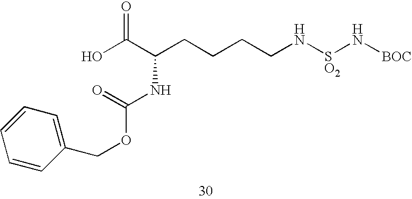 | 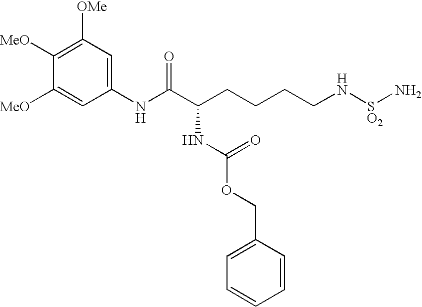 | (S)-benzyl 1-oxo-6- (sulfamoyl- amino)- 1-(3,4,5- trimethoxy- phenyl- amino)- hexan-2- ylcarbamate | (dmso-d6) d (ppm) 1 H: 9.94 (s, 1 H), 7.52 (d, J = 7.8 Hz, 1 H), 7.36-7.20 (m, 5 H), 6.99 (s, 2 H), 6.44 (s, 2 H), 6.43 (t, J = 3.5 Hz, 1 H), 5.01 (d, J = 2.5 Hz, 1 H), 3.72 (s, 6 H), 3.59 (s, 3 H), 2.82 (q, J = 6.3 Hz, 2 H), 1.70-1.23 (m, 6 H). | |
| #LRMS (ESI): (calc.) 524.2 (found) 525.2 (MH)+ | D, B | | | | | |
| 239 | 273 | 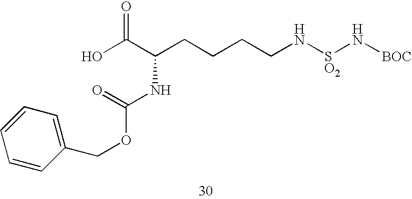 | 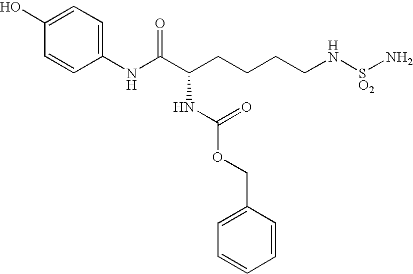 | (S)-benzyl 1- (4-hydroxy- phenyl amino)- 1-oxo-6- (sulfamoyl- amino)- hexan-2- ylcarbamate | (dmso-d6) d (ppm) 1 H: 9.73 (s, 1 H), 9.17 (s, 1 H), 7.47 (d, J = 7.8 Hz, 1 H), 7.36-7.21 (m, 7 H), 6.67 (d, J = 8.8 Hz, 2 H), 6.43 (s, 2 H), 6.42 (t, J = 6.1 Hz, 1 H), 5.01 (s, 2 H), 4.04 (q, J = 5.3 Hz, 1 H), 2.82 (q, J =6.5 Hz, 2 H), 1.69-1.52 (m, 2 H), | |
| #1.52-1.22 (m, 4 H). LRMS (ESI): (calc.) 450.2 (found) 451.3 (MH)+ | D, B | | | | | |
| 240 | 274 | 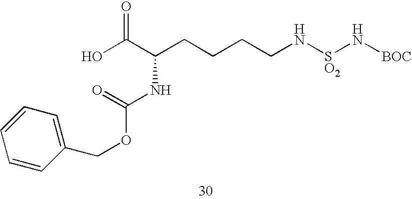 | 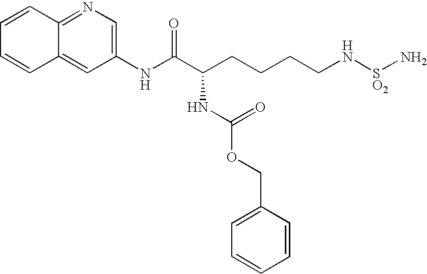 | (S)-benzyl 1-oxo-1- (quinolin-3- ylamino)-6- (sulfamoyl- amino)- hexan-2- ylcarbamate | (CD3OD) d (ppm) 1 H: 8.92 (d, J = 2.0 Hz, 1 H), 8.70 (s, 1 H), 7.97 (d, J =8.2 Hz, 1 H), 7.88 (d, J = 8.2 Hz, 1 H), 7.69 (t, J = 7.0 Hz, 1 H), 7.59 (t, J =7.6 Hz, 1 H), 7.39-7.25 (m, 4 H), 7.07 (s, 1 H), 5.12 (d, J = 3.3 Hz, 2 H), 4.31 (dd, J = 9.0, 5.3 | |
| # Hz, 1 H), 3.04 (t, J = 6.7 Hz, 2 H), 1.98-1.73 (m, 2 H), 1.69-1.43 (m, 4 H). LRMS (ESI): (calc.) 485.2 (found) 486.2 (MH)+ | D, B | | | | | |
| 241 | 275 | 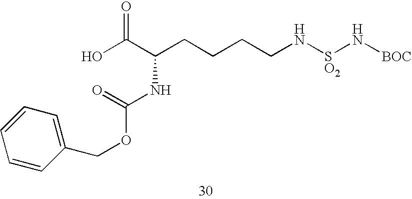 | 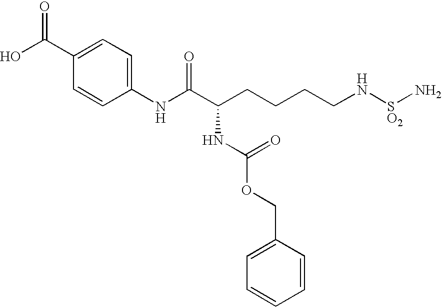 | (S)-4-(2- (benzyl- oxy- carbonyl amino)-6- (sulfamoyl- amino)hex- anamido)- benzoic acid | (dmso) d (ppm) 1 H: 10.33 (s, 1 H), 7.87 (d, J = 9.0 Hz, 2 H), 7.69 (d, J = 8.8 Hz, 2 H), 7.61 (d, J =7.6 Hz, 1 H), 7.37-7.13 (m, 5 H), 6.43 (s, 2 H), 6.42 (t, J = 3.5 Hz, 1 H), 5.01 (s, 2 H), 4.11 (q, J = 4.9 Hz, 1 H), 2.82 (q, J = 6.3 Hz, 2 H), | |
| # 1.75-1.53 (m, 2 H), 1.53-1.22 (m, 4 H). LRMS (ESI): (calc.) 478.2 (found) 479.2 (MH)+ | D, B | | | | | |
| 242 | 276 | 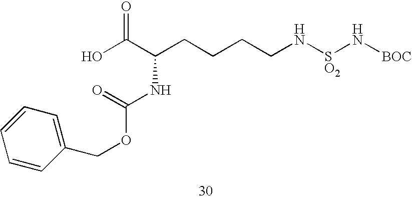 | 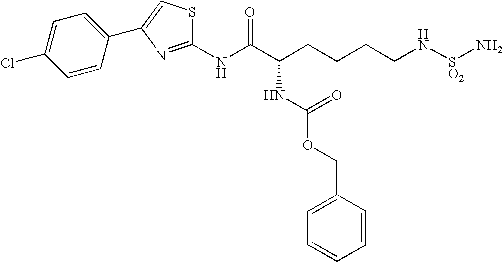 | (S)-benzyl 1-(4-(4- chloro- phenyl)- thiazol-2- ylamino)- 1-oxo-6- (sulfamoyl- amino)- hexan-2- ylcarbamate | (CD3OD) d (ppm) 1 H: 7.89 (d, J = 8.6 Hz, 2 H), 7.42-7.22 (m, 8 H), 5.10 (dd, J = 19, 13 Hz, 2 H), 4.34 (dd, J = 8.8, 5.1 Hz, 1 H), 3.03 (t, J = 6.7 Hz, 2 H), 1.93-1.69 (m, 2 H), 1.67-1.43 (m, 4 H). LRMS (ESI): (calc.) 551.1 (found) 552.1 | |
| #(MH)+ | D, B | | | | | |
| 243 | 277 | 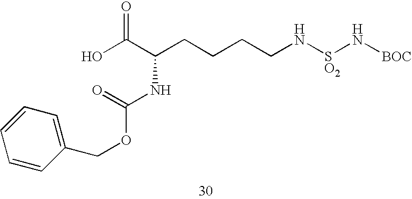 | 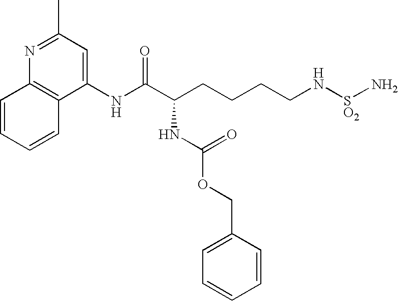 | (S)-benzyl 1-(2-methyl- quinolin-4- ylamino)- 1-oxo-6- (sulfamoyl- amino)- hexan-2- ylcarbamate | (CD3OD) d (ppm) 1 H: 8.12 (d, J = 8.6 Hz, 1 H), 8.01 (s, 1 H), 7.95 (d, J =8.4 Hz, 1 H), 7.76 (t, J = 7.4 Hz, 1 H), 7.57 (t, J = 7.6 Hz, 1 H), 7.41-7.23 (m, 4 H), 7.09 (s, 1 H), 5.15 (s, 2 H), 4.46 (dd, J = 9.0, 5.7 Hz, 1 H), 3.05 (t, J = 6.7 Hz, | |
| # 2 H), 2.69 (s, 3 H), 2.01-1.74 (m, 4 H), 1.71-1.50 (m, 6 H). LRMS (ESI): (calc.) 499.2 (found) 500.2 (MH)+ | D, B | | | | | |
| 244 | 278 | 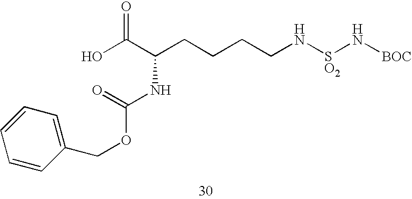 | 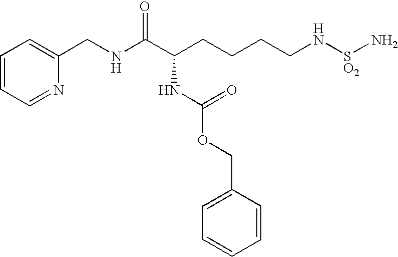 | (S)-benzyl 1-oxo-1- (pyridin-2- ylmethyl- amino)-6- (sulfamoyl- amino)- hexan-2- ylcarbamate | (CD3OD) d (ppm) 1 H: 8.47 (d, J = 4.5 Hz, 1 H), 7.83 (t, J = 7.8 Hz, 1 H), 7.43 (d, J = 8.0 Hz, 1 H), 7.38-7.27 (m, 6 H), 5.11 (s, 2 H), 4.51 (s, 2 H), 4.14 (dd, J = 8.8, 5.3 Hz, 1 H), 3.02 (t, J =6.8 Hz, 2 H), 1.90-1.78 (m, 1 H), 1.76-1.63 (m, 1 H), | |
| #1.63-1.40 (m, 4 H). LRMS (ESI): (calc.) 449.2 (found) 450.2 (MH)+ | D, B | | | | | |
| 245 | 279 | 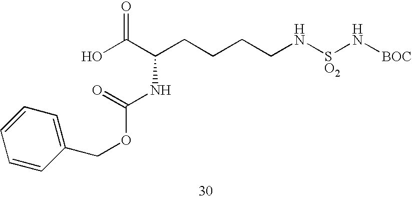 | 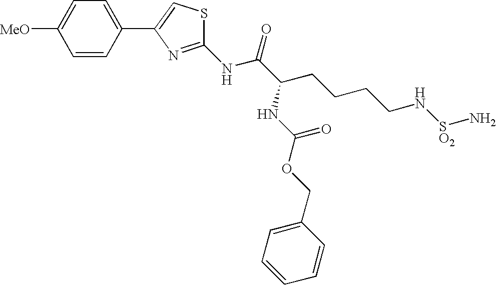 | (S)-benzyl 1- (4-(4-meth- oxyphenyl)- thiazol-2- ylamino)-1- oxo-6- (sulfamoyl- amino)- hexan-2- ylcarbamate | (CD3OD) d (ppm) 1 H: 7.82 (d, J = 9.0 Hz, 2 H), 7.39-7.29 (m, 4 H), 7.22 (s, 1 H), 7.13 (bs, 1 H), 6.95 (d, J = 9.0 Hz, 2 H), 5.13 (d, J = 13 Hz, 1 H), 5.09 (d, J = 13 Hz, 1 H), 4.34 (dd, J = 8.8, 5.5 Hz, 1 H), 3.82 (s, 3 H), 3.03 (t, J | |
| # =6.7 Hz, 2 H), 1.93-1.70 (m, 2 H), 1.67-1.43 (m, 4 H). LRMS (ESI): (calc.) 547.2 (found) 548.3 (MH)+ | D, B | | | | | |
| 246 | 280 | 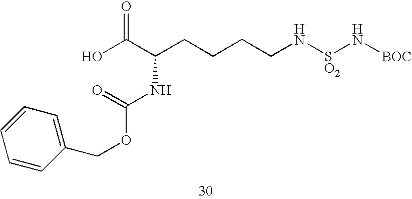 | 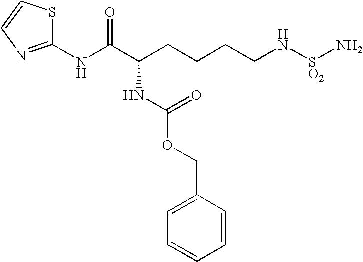 | (S)-benzyl 1-oxo-6- (sulfamoyl- amino)- 1-(thiazol-2- ylamino)- hexan-2- ylcarbamate | (dmso-d6) d (ppm) 1 H: 12.23 (s, 1 H), 7.68 (d, J = 7.4 Hz, 1 H), 7.47 (d J = 3.5 Hz, 1 H), 7.36-7.29 (m, 4 H), 7.22 (d, J = 3.5 Hz, 1 H), 7.19-7.12 (m, 1 H), 6.44 (bs, 3 H), 5.01 (s, 2 H), 4.23 (q, J =4.7 Hz, 1 H), 2.84-2.80 (d, 2 H), 1.70-1.57 (m, 2 H), 1.57-1.23 (m, | |
| # 4 H). LRMS (ESI): (calc.) 441.1 (found) 442.1 (MH)+ | D, B | | | | | |
| 247 | 281 | 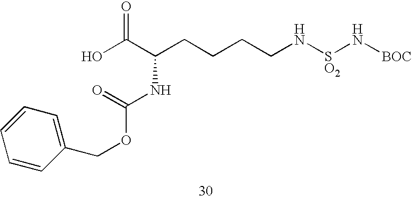 | 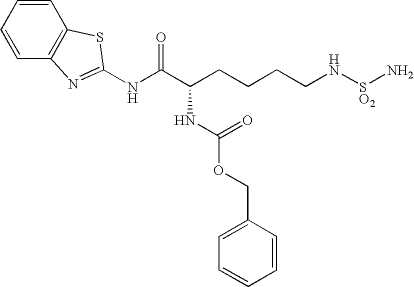 | (S)-benzyl 1- (benzo[d]- thiazol-2- ylamino)- 1-oxo-6- (sulfamoyl- amino)- hexan-2- ylcarbamate | (dmso-d6) d (ppm) 1 H: 12.51 (s, 1 H), 7.97 (d, J = 7.8 Hz, 1 H), 7.74 (d, J =8.2 Hz, 2 H), 7.43 (t, J = 7.0 Hz, 1 H), 7.41-7.10 (m, 6 H), 6.44 (s, 2 H), 6.43 (t, J = 3.1 Hz, 1 H), 5.03 (s, 2 H), 4.28 (q, J = 4.7 Hz, 1 H), 2.83 (q, J = 6.3 Hz, 2 | |
| # H), 1.78-1.59 (m, 2 H), 1.53-1.29 (m, 4 H). LRMS (ESI): (calc.) 491.1 (found) 492.2 (MH)+ | D, B | | | | | |
| 248 | 282 | 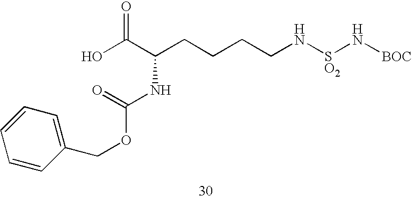 | 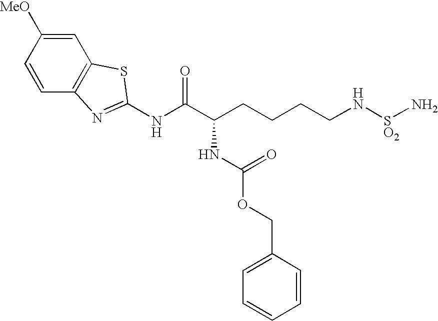 | (S)-benzyl 1- (6-methoxy- benzo[d]- thiazol-2- ylamino)-1- oxo-6- (sulfamoyl- amino)- hexan-2- ylcarbamate | (dmso-d6) d (ppm) 1 H: 12.36 (s, 1 H), 7.72 (d, J = 7.4 Hz, 1 H), 7.62 (d, J =8.8 Hz, 1 H), 7.56 (d, J = 2.5 Hz, 1 H), 7.36-7.29 (m, 4 H), 7.22-7.04 (m, 1 H), 7.02 (dd, J = 9.0, 2.7 Hz, 1 H), 6.44 (s, 2 H), 6.43 (t, J = 6.1 Hz, 1 H), 5.02 | |
| # (s, 2 H), 4.26 (q, J = 5.1 Hz, 1 H), 3.79 (s, 3 H), 2.83 (q, J = 6.5 Hz, 2 H), 1.78-1.59 (m, 2 H), 1.52-1.28 (m, 2 H). LRMS (ESI): (calc.) 521.1 (found) 522.2 (MH)+ | D, B | | | | | |
| 249 | 283 | 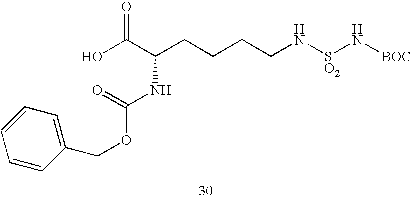 | 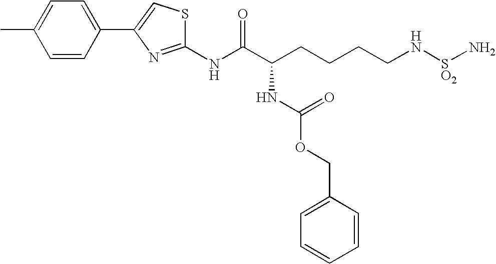 | (S)-benzyl 1-oxo-6- (sulfamoyl- amino)- 1-(4-p-tolyl- thiazol-2- ylamino)- hexan-2- ylcarbamate | (dmso-d6) d (ppm) 1 H: 12.36 (s, 1 H), 7.78 (d, J = 8.2 Hz, 2 H), 7.69 (d, J =7.4 Hz, 1 H), 7.54 (s, 1 H), 7.37-7.30 (m, 4 H), 7.23 (d, J = 8.0 Hz, 2 H), 7.20-7.10 (m, 1 H), 6.44 (s, 2 H), 6.43 (t, J = 6.3 Hz, 1 H), 5.02 (s, 2 H), 4.25 (q, J = 7.8 Hz, 1 | |
| # H), 2.83 (q, J = 6.7 Hz, 2 H), 2.32 (s, 3 H), 1.79-1.58 (m, 2 H), 1.58-1.24 (m, 4 H). LRMS (ESI): (calc.) 531.2 (found) 532.2 (MH)+ | D, B | | | | | |
| 250 | 284 | 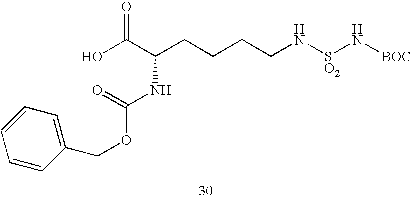 | 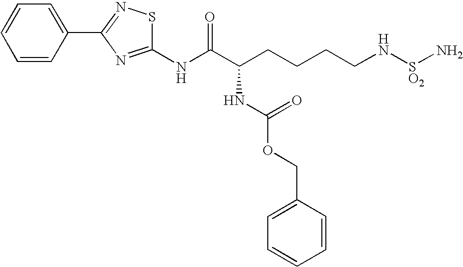 | (S)-benzyl 1-oxo-1- (3-phenyl- 1,2,4- thiadiazol-5- ylamino)-6- (sulfamoyl- amino)- hexan-2- ylcarbamate | (dmso-d6) d (ppm) 1 H: 13.29 (s, 1 H), 8.16 (dd, 7.8, 2.5 Hz, 2 H), 7.83 (s, 1 H), 7.52-7.49 (m, 3 H), 7.40-7.29 (m, 4 H), 7.10-7.04 (m, 1 H), 6.44 (s, 2 H), 6.42 (t, J = 8.2 Hz, 1 H), 5.03 (s, 2 H), 4.38-4.29 (m, 1 H), 2.84 (q, J = 6.3 Hz, 2 H), 1.80-1.60 | |
| # (m, 2 H), 1.57-1.30 (m, 4 H). LRMS (ESI): (calc.) 518.1 (found) 519.1 (MH)+ | D, B | | | | | |
| 251 | 285 | 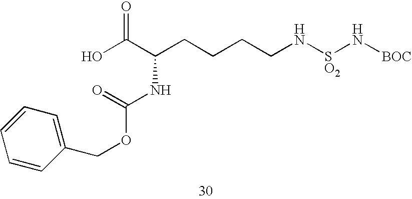 | 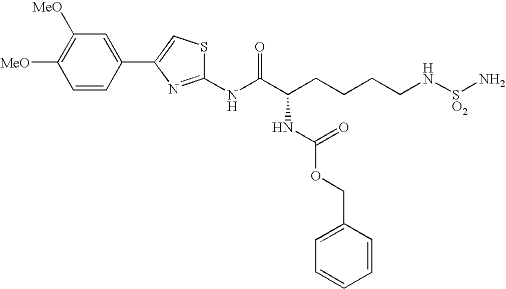 | (S)-benzyl 1-(4-(3,4- dimethoxy- phenyl)- thiazol-2- ylamino)-1- oxo-6- (sulfamoyl- amino)- hexan-2- ylcarbamate | (dmso-d6) d (ppm) 1 H: 12.36 (s, 1 H), 7.68 (d, J = 7.2 Hz, 1 H), 7.51 (s, 1 H), 7.45-7.42 (m, 2 H), 7.36-7.28 (m, 4 H), 7.12-7.06 (m, 1 H), 6.99 (d, J = 8.6 Hz, 1 H), 6.44 (s, 2 H), 6.43 (t, J = 6.1 Hz, 1 H), 5.01 (d, J =2.0. Hz, 2 H), | |
| # 4.25 (q, J = 5.5 Hz, 1 H), 3.80 (s, 3 H), 3.77 (s, 3 H), 2.83 (q, J =6.3 Hz, 2 H), 1.76-1.58 (m, 2 H), 1.56-1.27 (m, 4 H). LRMS (ESI): (calc.) 577.2 (found) 578.2 (MH)+ | D, B | | | | | |
| 252 | 286 | 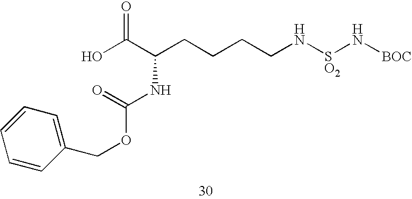 | 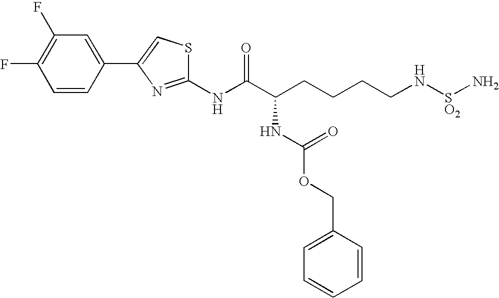 | (S)-benzyl 1-(4-(3,4- difluoro- phenyl)- thiazol-2- ylamino)-1- oxo-6- (sulfamoyl- amino)- hexan-2- ylcarbamate | (dmso-d6) d (ppm) 1 H: 12.42 (s, 1 H), 7.93-7.87 (m, 1 H), 7.73-7.69 (m, 3 H), 7.51 (q, J = 11 Hz, 1 H), 7.38-7.28 (m, 4 H), 7.21-7.10 (m, 1 H), 6.44 (s, 2 H), 6.42 (t, J = 6.1 Hz, 1 H), 5.02 (d, J =2.0 Hz, 2 H), 4.24 (q, J = 5.1 Hz, 1 H), | |
| #2.82 (q, J = 6.5 Hz, 2 H), 1.76-1.55 (m, 2 H), 1.55-1.23 (m, 4 H) LRMS (ESI): (calc.) 553.1 (found) 554.2 (MH)+ | D, B | | | | | |
| 253 | 287 | 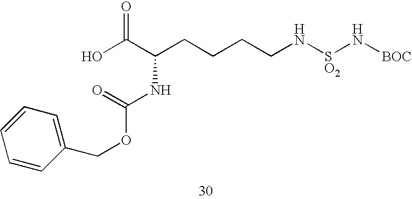 | 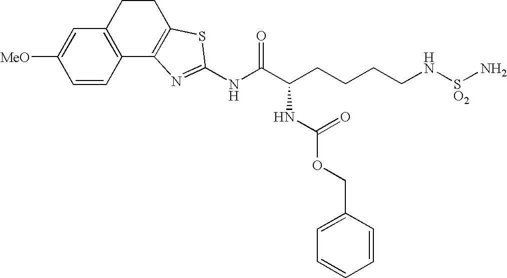 | (S)-benzyl 1- (7-methoxy- 4,5-dihydro- naphtho[1,2- d]thiazol-2- ylamino)-1- oxo-6- (sulfamoyl- amino)- hexan-2- ylcarbamate | (CD3CN) d (ppm) 1 H: 7.64 (d, J = 8.2 Hz, 1 H), 7.40-7.35 (m, 4 H), 6.87-6.82 (m, 2 H), 6.15 (bd, J = 7.0 Hz, 1 H), 5.19-5.09 (m, 4 H), 5.08-5.00 (m, 1 H), 4.30 (q, J = 7.4 Hz, 1 H), 3.82 (s, 3 H), 3.04-2.91 (m, 6 H), 2.00-1.81 | |
| # (m, 1 H), 1.80-1.68 (m, 1 H), 1.63-1.40 (m, 4 H). LRMS (ESI): (calc.) 573.2 (found) 574.3 (MH)+ | D, B | | | | | |
| 254 | 288 | 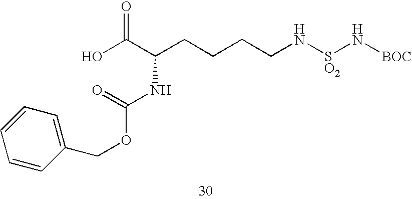 | 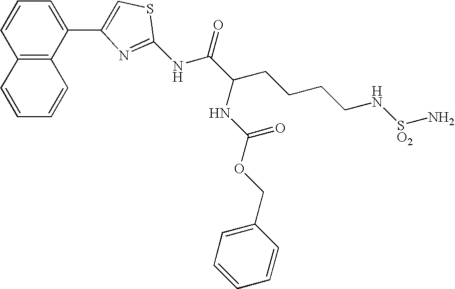 | benzyl 1-(4- (naphthalen- 1-yl)thiazol- 2-ylamino)- 1-oxo-6- (sulfamoyl- amino)- hexan-2- ylcarbamate | (CD3CN) d (ppm) 1 H: 8.33 (d, J = 6.8 Hz, 1 H), 7.97 (t, J = 8.0 Hz, 2 H), 7.69 (d, J = 7.2 Hz, 1 H), 7.59-7.52 (m, 3 H), 7.41-7.35 (m, 4 H), 7.26 (s, 1 H), 6.18 (bc, J = 7.4 Hz, 1 H), 5.20-2.00 (m, 5 H), 4.35 (q, J = 8.6 Hz, 1 H), 3.00 (q, J = | |
| # 6.5 Hz, 2 H), 1.96-1.84 (m, 1 H), 1.84-1.69 (m, 1 H), 1.64-1.41 (m, 4 H). LRMS (ESI): (calc.) 567.2 (found) 568.2 (MH)+ | D, B | | | | | |
| 255 | 289 | 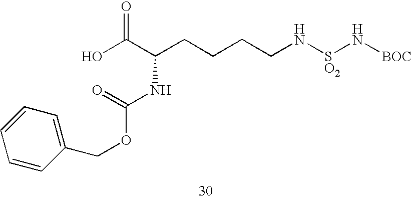 | 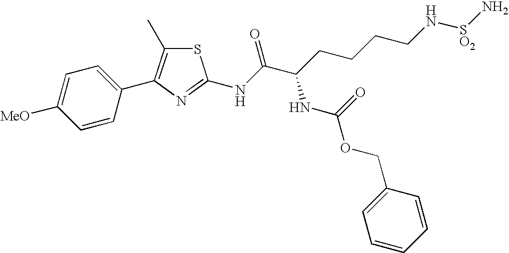 | (S)-benzyl 1-(4-(4- methoxy- phenyl)-5- methyl- thiazol-2- ylamino)- 1-oxo-6- (sulfamoyl- amino)- hexan-2- ylcarbamate | (CD3CN) d (ppm) 1 H: 7.59 (d, J = 9.0 Hz, 2 H), 7.41-7.32 (m, 4 H), 7.01 (d, J = 9.0 Hz, 2 H), 6.14 (bc, J = 7.4 Hz, 1 H), 5.19-5.00 (m, 5 H), 4.30 (q, J = 5.1 Hz, 1 H), 3.85 (s, 3 H), 2.99 (q, J = 6.5 Hz, 2 H), 2.47 (s, 3 | |
| # H), 1.94-1.80 (m, 1 H), 1.79-1.63 (m, 1 H), 1.62-1.41 (m, 4 H). LRMS (ESI): (calc.) 561.2 (found) 562.3 (MH)+ | D, B | | | | | |
| 256 | 290 | 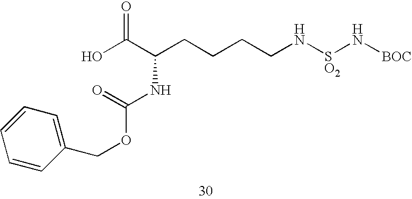 | 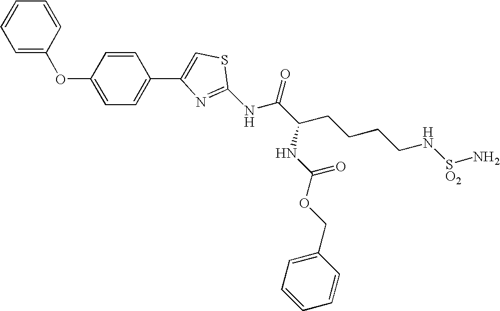 | (S)-benzyl 1- oxo-1-(4-(4- phenoxy- phenyl)- thiazol-2- ylamino)-6- (sulfamoyl- amino)- hexan-2- ylcarbamate | (CD3CN) d (ppm) 1 H: 7.90 (d, J = 8.8 H, 2 H), 7.45-7.36 (m, 7 H), 7.29 (s, 1 H), 7.19 (t, J = 7.2 Hz, 1 H), 7.09-7.04 (m, 4 H), 6.15 (bd, J = 7.2 Hz, 1 H), 5.16-5.01 5 H), 4.33 (q, J = 5.3 Hz, 1 H), 3.00 (q, J =6.5 Hz, 2 H), 1.94-1.82 | |
| # (m, 1 H), 1.82-1.69 (m, 1 H), 1.63-1.40 (m, 4 H). LRMS (ESI): (calc.) 609.3 (found) 610.4 (MH)+ | D, B | | | | | |
| 257 | 291 | 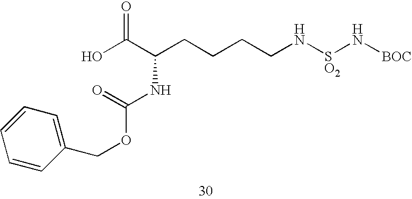 | 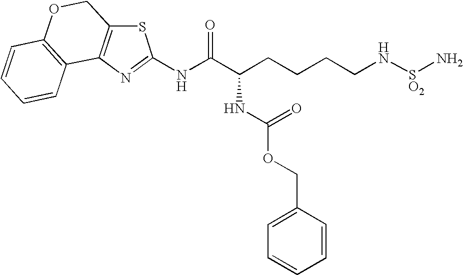 | (S)-benzyl 1-(4H- chromeno- [4,3-d]- thiazol-2- ylamino)- 1-oxo-6- (sulfamoyl- amino)- hexan-2- ylcarbamate | (CD3CN) d (ppm) 1 H: 7.64 (dd, J =7.6, 1.6 Hz, 1 H), 7.41-7.36 (m, 5 H), 7.22 (td, J = 7.6, 1.8 Hz, 1 H), 7.04 (td, J = 7.4, 1.0 Hz, 1 H), 6.94 (dd, J =8.0, 0.8 Hz, 1 H), 6.15 (bd, J = 6.3 Hz, 1 H), 5.44 (s, 2 H), 5.16-5.02 (m, 5 | |
| # H), 4.33 (q, J =4.3 Hz, 1 H), 3.01 (q, J = 6.6 Hz, 2 H), 1.94-1.82 (m, 1 H), 1.82-1.69 (m, 1 H), 1.64-1.40 (m, 4 H). LRMS (ESI): (calc.) 545.2 (found) 546.4 (MH)+ | D, B | | | | | |
| 258 | 292 | 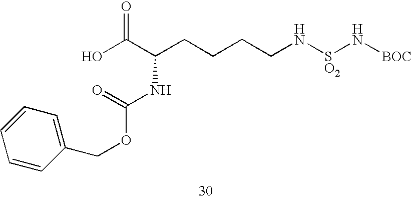 | 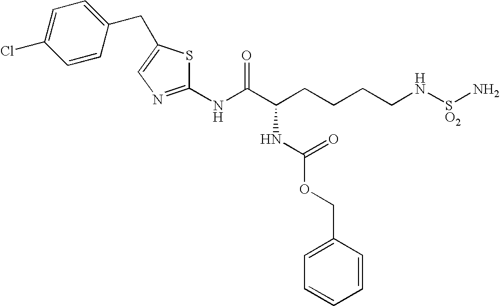 | (S)-benzyl 1-(5-(4- chloro- benzyl)- thiazol-2- ylamino)- 1-oxo-6- (sulfamoyl- amino)- hexan-2- ylcarbamate | (CD3CN) d (ppm) 1 H: 7.66-7.27 (m, 9 H), 7.20 (s, 1 H), 6.08 9 t, J = 7.0 Hz, 1 H), 5.18-4.98 (m, 5 H), 4.26 (q, J = 4.3 Hz, 1 H), 4.10 (s, 2 H), 2.98 (q, J = 6.5 Hz, 2 H), 1.92-1.78 (m, 1 H), 1.77-1.63 (m, 1 H), 1.63-1.39 (m, 4 H). LRMS (ESI): | |
| # (calc.) 565.2 (found) 566.4 (MH)+ | D, B | | | | | |
| 259 | 293 | 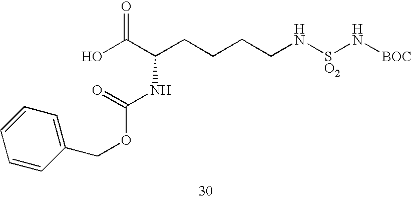 | 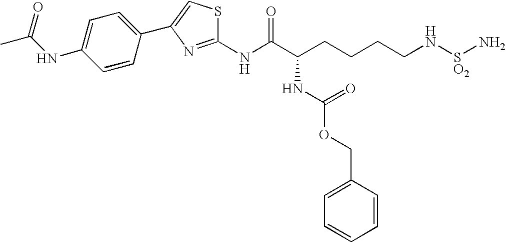 | (S)-benzyl 1-(4-(4- acetamido- phenyl)- thiazol-2- ylamino)-1- oxo-6- (sulfamoyl- amino)- hexan-2- ylcarbamate | (dmso-d6) d (ppm) 1 H: 12.37 (s, 1 H), 10.01 (s, 1 H), 7.80 (d, J = 8.8 Hz, 2 H), 7.68 (d, J = 7.2 Hz, 1 H), 7.62 (d, J =8.8 Hz, 2 H), 7.49 (s, 1 H), 7.36-7.29 (m, 4 H), 7.13-7.11 (m, 1 H), 6.44 (s, 2 H), 6.43 (t, J = 6.1 Hz, 1 H), 5.02 | |
| # (d, J = 2.0 Hz, 2 H), 4.25 (q, J = 4.9 Hz, 1 H), 2.83 (q, J =6.5 Hz, 2 H), 2.04 (s, 3 H), 1.78-1.53 (m, 2 H), 1.51-1.24 (m, 4 H). LRMS (ESI): (calc.) 574.2 (found) 575.2 (MH)+ | D, B | | | | | |
| 260 | 294 | 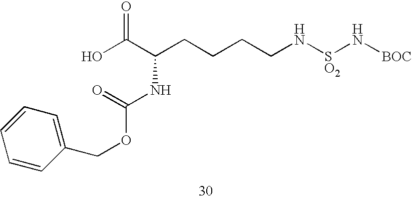 | 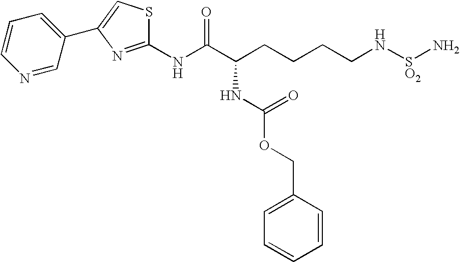 | (S)-benzyl 1-oxo-1-(4- (pyridin-3- yl)thiazol-2- ylamino)-6- (sulfamoyl- amino)- hexan-2- ylcarbamate | (dmso-d6) d (ppm) 1 H: 12.48 (s, 1 H), 9.11 (s, 1 H), 8.53 (s, 1 H), 8.25 (s, 1 H), 7.83 (s, 1 H), 7.71 (d, J = 7.2 Hz, 1 H), 7.49 (q, J =4.5 Hz, 1 H), 7.36-7.11 (m, 5 H), 6.44 (s, 2 H), 6.45-6.42 (m, 1 H), 5.02 (q, J = 2.0 Hz, 2 H), 4.26 (q, J =5.1 | |
| # Hz, 1 H), 2.83 (q, J = 5.7 Hz, 2 H), 1.78-1.57 (m, 2 H), 1.55-1.26 (m, 4 H). LRMS (ESI): (calc.) 518.1 (found) 519.2 (MH)+ | D, B | | | | | |
| 261 | 295 | 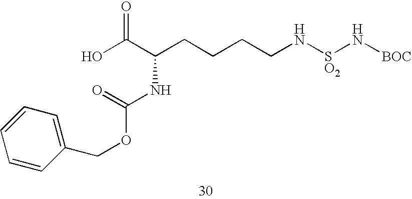 | 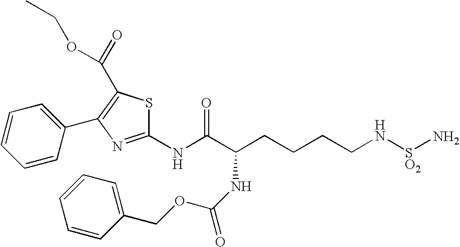 | (S)-ethyl 2- (2-(benzyl- oxycarbonyl amino)-6- (sulfamoyl- amino)- hex- anamido)-4- phenyl- thiazole-5- carboxylate | (dmso-d6) d (ppm) 1 H: 12.83 (s, 1 H), 7.75 (d, J = 6.3 Hz, 1 H), 7.70-7.67 (m, 2 H), 7.43-7.40 (m, 3 H), 7.36-7.29 (m, 4 H), 7.21-7.11 (m, 1 H), 6.44 (s, 2 H), 6.43 9 t, J = 6.1 Hz, 1 H), 5.02 (s, 2 H), 4.24 (q, J = 6.1 Hz, 1 H), 4.17 | |
| # (q, J = 7.0 Hz, 2 H), 2.82 (q, J = 6.5 Hz, 2 H), 1.77-1.55 (m, 2 H), 1.53-1.23 (m, 4 H), 1.19 (t, J =7.0 Hz, 3 H). LRMS (ESI): (calc.) 589.2 (found) 590.2 (MH)+ | D, B | | | | | |
| 262 | 296 | 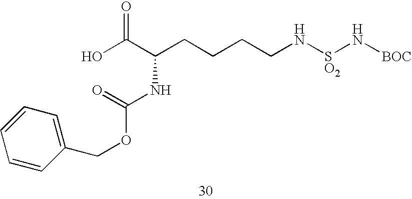 | 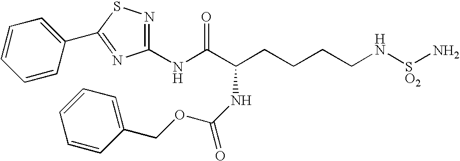 | (S)-benzyl 1-oxo-1- (5-phenyl- 1,2,4- thiadiazol-3- ylamino)-6- (sulfamoyl- amino)- hexan-2- ylcarbamate | (dmso-d6) d (ppm) 1 H: 11.50 (s, 1 H), 7.97 (d, J = 6.7 Hz, 2 H), 7.65-7.56 (m, 4 H), 7.40-7.16 (m, 5 H), 6.44 (s, 2 H), 6.43 (t, J = 4.9 Hz, 1 H), 5.02 (d, J =1.6 Hz, 2 H), 4.32-4.20 (m, 1 H), 2.83 (q, J = 6.3 Hz, 2 H), 1.77-1.56 (m, 2 H), | |
| # 1.56-1.28 (m, 4 H). LRMS (ESI): (calc.) 518.1 (found) 519.2 (MH)+ | U, B | | | | | |
| 263 | 297 | 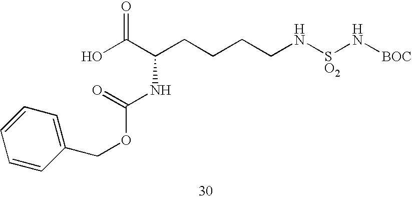 | 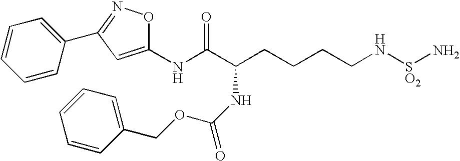 | (S)-benzyl 1-oxo-1- (3-phenyl- isoxazol- 5-ylamino)- 6-(sulfa- moylamino)- hexan-2- ylcarbamate | (dmso-d6) d (ppm) 1 H: 11.88 (s, 1 H), 7.85-7.83 (m, 2 H), 7.72 (d, J = 7.2 Hz, 1 H), 7.50-7.48 (m 3 H), 7.36-7.17 (m 5 H), 6.72 (s, 1 H), 6.44 (s, 2 H), 6.44 (t, J = 8.6 Hz, 1 H), 5.02 (s, 2 H), 4.15 (q, J = 4.9 Hz, 1 H), 2.83 (q, J = 6.5 Hz, 2 H), | |
| # 1.76-1.54 (m, 2 H), 1.52-1.26 (m 4 H). LRMS (ESI): (calc.) 501.2 (found) 502.2 (MH)+ | U, B | | | | | |
| 264 | 298 | 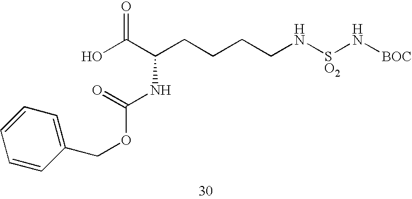 | 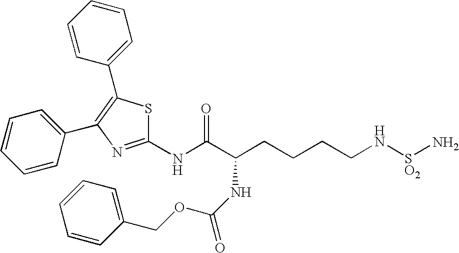 | (S)-benzyl 1-(4,5- diphenyl- thiazol-2- ylamino)- 1-oxo-6- (sulfamoyl- amino)- hexan-2- ylcarbamate | (dmso-d6) d (ppm) 1 H: 12.48 (s, 1 H), 7.71 (d, J = 7.1 Hz, 1 H), 7.42-7.11 (m, 15 H), 6.44 (s, 2 H), 6.44 (t, J = 5.9 Hz, 1 H), 5.02 (s, 2 H), 4.26 (q, J = 5.5 Hz, 1 H), 2.84 (q, J =6.5 Hz, 2 H), 1.76-1.58 (m, 2 H), 1.53-1.27 (m, 4 H). LRMS (ESI): | |
| # (calc.) 593.2 (found) 594.3 (MH)+ | U, B | | | | | |
| 265 | 299 | 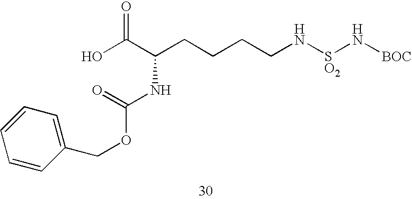 | 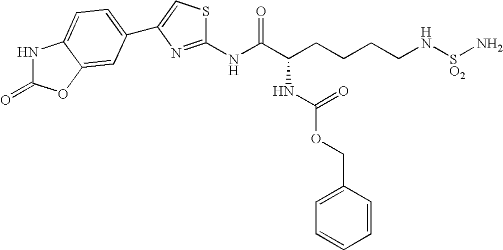 | (S)-benzyl 1-oxo-1-(4- (2-oxo-2,3- dihydro- benzo[d]- oxazol-6- yl)thiazol-2- ylamino)-6- (sulfamoyl- amino)- hexan-2- ylcarbamate | (dmso-d6) d (ppm) 1 H: 12.38 (s, 1 H), 11.72 (s, 1 H), 7.76 (s, 1 H), 7.71 (t, J =8.4 Hz, 2 H), 7.59 (s, 1 H), 7.36-7.11 (m, 6 H), 6.44 (s, 2 H), 6.44 (t, J = 3.9 Hz, 1 H), 5.02 (s, 2 H), 4.25 (q, J = 5.1 Hz, 1 H), 2.82 | |
| # (q, J = 6.3 Hz, 2 H), 1.78-1.57 (m, 2 H), 1.55-1.26 (m, 4 H). LRMS (ESI): (calc.) 574.1 (found) 575.2 (MH)+ | U, B | | | | | |
| 266 | 300 | 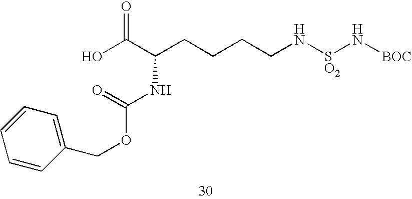 | 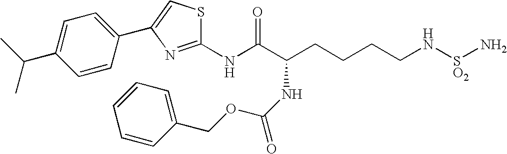 | (S)-benzyl 1-(4-(4- isopropyl- phenyl)- thiazol-2- ylamino)-1- oxo-6- (sulfamoyl- amino)- hexan-2- ylcarbamate | (CD3CN) d (ppm) 1 H: 7.83 (d, J = 8.2 Hz, 2 H), 7.41-7.25 (m, 8 H), 6.16 (d, J = 7.0 Hz, 1 H), 5.18-5.12 (m, 2 H), 5.13 (s, 2 H), 5.02 (m, 1 H), 4.33 (q, J = 5.9 Hz, 1 H), 3.02-2.93 (m, 3 H), 1.95-1.83 (m, 1 H), 1.81-1.69 (m, 1 H), 1.64-1.42 | |
| # (m, 4 H), 1.28 (d, J = 7.0 Hz, 6 H). LRMS (ESI): (calc.) 559.2 (found) 560.3 (MH)+ | U, B | | | | | |
| 267 | 301 | 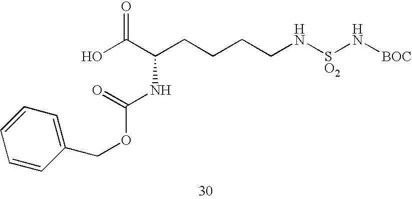 | 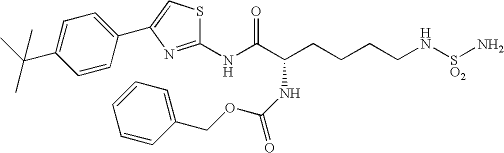 | (S)-benzyl 1-(4-(4-tert- butyl- phenyl)- thiazol-2- ylamino)- 1-oxo-6- (sulfamoyl- amino)- hexan-2- ylcarbamate | (dmso-d6) d (ppm) 1 H: 12.39 (s, 1 H), 7.80 (q, J = 8.4 Hz, 2 H), 7.68 (d, J =7.2 Hz, 1 H), 7.54 (s, 1 H), 7.43 (d, J =8.4 Hz, 2 H), 7.36-7.12 (m, 5 H), 6.44 (s, 2 H), 6.43 (t, J = 4.5 Hz, 1 H), 5.01 (d, J = 1.8 Hz, 2 H), 4.25 (q, | |
| #J = 4.9 Hz, 1 H), 2.83 (q, J = 6.3 Hz, 2 H), 1.75-1.57 (m, 2 H), 1.57-1.28 (m, 4 H), 1.29 (s, 9 H). LRMS (ESI): (calc.) 573.2 (found) 574.3 (MH)+ | U, B | | | | | |
| 268 | 302 | 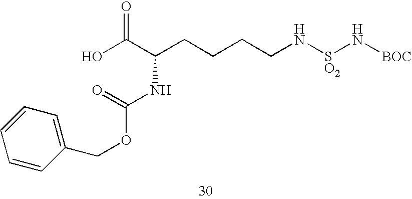 | 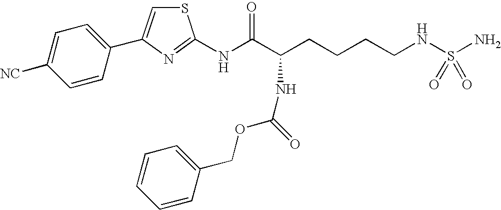 | (S)-benzyl 1-(4-(4- cyano- phenyl)- thiazol-2- ylamino)- 1-oxo-6- (sulfamoyl- amino)- hexan-2- ylcarbamate | (DMSO-d6) d (ppm) 1 H: 12.48 (s, 1 H), 8.07 (d, J = 7.8 Hz, 2 H), 7.93 (s, 1 H), 7.89 (d, J = 7.6 Hz, 2 H), 7.71 (d, J =7.4 Hz, 1 H), 7.36-7.10 (m, 5 H), 5.01 (s, 2 H), 4.25 (m, 1 H), 2.82 (m. 2 H) 1.65 (m, 2 H), 1.48-1.22 (m, 4 H) LRMS (ESI): | |
| # (calc.) 542.6 (found) 543.2 (MH)+ | U, B | | | | | |
| 269 | 303 | 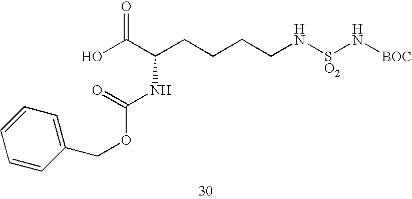 | 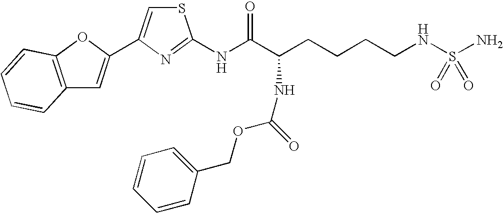 | (S)-benzyl 1-(4-(benzo- furan-2- yl)thiazol-2- ylamino)- 1-oxo-6- (sulfamoyl- amino)- hexan-2- ylcarbamate | (CD3OD) d (ppm) 1 H: 7.61 (d, 7.4 Hz, 1 H), 7.5 (overlapped signals, 2 H), 7.39-7.21 (m, 6 H), 7.13 (m, 1 H), 7.09 (s, 1 H), 5.12 (d, J = 5.8 Hz, 2 H), 4.35 (m, 1 H), 3.3 (t, J = 6.6 Hz, 2 H), 1.87 (m, 1 H), 1.77 (m, 1 H), 1.66-1.42 (m, 4 H). LRMS | |
| #(ESI): (calc.) 557.6 (found) 558.2 (MH)+ | U, B | | | | | |
| 270 | 304 | 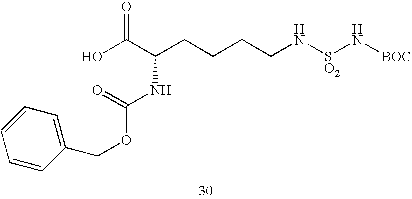 | 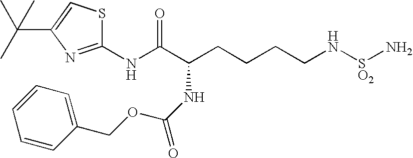 | (S)-benzyl 1-(4-tert- butyl- thiazol-2- ylamino)- 1-oxo-6- (sulfamoyl- amino)- hexan-2- ylcarbamate | (dmso-d6) d (ppm) 1 H: 12.18 (s, 1 H), 7.63 (d, J = 7.4 Hz, 1 H), 7.37-7.08 (m, 5 H), 6.73 (s, 2 H), 6.42 (t, J = 6.1 Hz, 1 H), 5.01 (d, J =1.8 Hz, 2 H), 4.20 (q, J = 5.3 Hz, 1 H), 2.82 (q, J = 6.3 Hz, 2 H), 1.72-1.53 (m, 2 H), 1.52-1.22 (m, 4 H), | |
| # 1.25 (s, 9 H). LRMS (ESI): (calc.) 497.2 (found) 498.2 (MH)+ | U, B | | | | | |
| 271 | 305 | 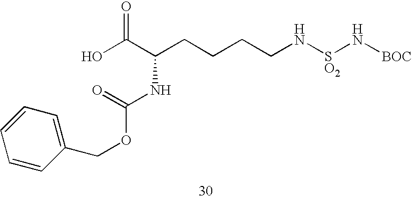 | 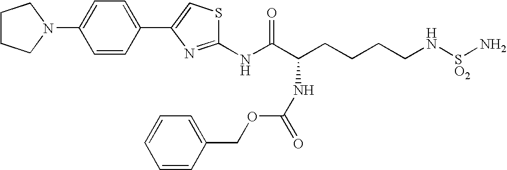 | (S)-benzyl 1- oxo-1-(4-(4- (pyrrolidin- 1-yl)phenyl)- thiazol-2- ylamino)-6- (sulfamoyl- amino)- hexan-2- ylcarbamate | (dmso-d6) d (ppm) 1 H: 12.28 (s, 1 H), 7.70-7.65 (m, 3 H), 7.36-7.13 (m, 5 H), 7.24 (s, 1 H), 6.55 (d, J 8.8 Hz, 2 H), 6.44 (bs, 3 H), 5.01 (d, J = 2.0 Hz, 2 H), 4.24 (q, J = 4.7 Hz, 1 H), 3.28-3.23 (m, 4 H), 2.83 (q, J = 5.9 Hz, 2 H), | |
| #1.97-1.89 (m, 4 H), 1.76-1.55 (m, 2 H), 1.53-1.26 (m, 4 H). LRMS (ESI): (calc.) 586.2 (found) 587.3 (MH)+ | U, B | | | | | |
| 272 | 306 | 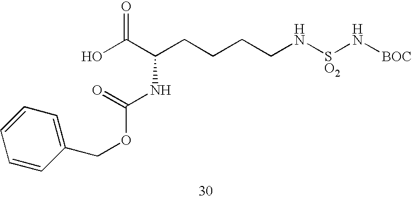 | 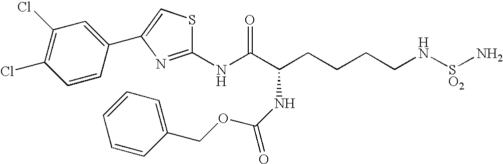 | (S)-benzyl 1-(4-(3,4- dichloro- phenyl)- thiazol-2- ylamino)- 1-oxo-6- (sulfamoyl- amino)- hexan-2- ylcarbamate | (dmso-d6) d (ppm) 1 H: 12.44 (s, 1 H), 8.14 (s, 1 H), 7.89-7.84 (m, 2 H), 7.72-7.68 (m, 2 H), 7.38-7.11 (m, 5 H), 6.44 (s, 2 H), 6.43 (t, J = 6.3 Hz, 1 H), 5.02 (d, j = 2.2 Hz, 2 H), 4.25 (q, J =5.3 Hz, 1 H), 2.83 (q, J = 6.3 Hz, 2 H), | |
| #1.75-1.58 (m, 2 H), 1.56-1.27 (m, 4 H). LRMS (ESI): (calc.) 585.1 (found) 584.1 (M − H)− | U, B | | | | | |
| 273 | 307 | 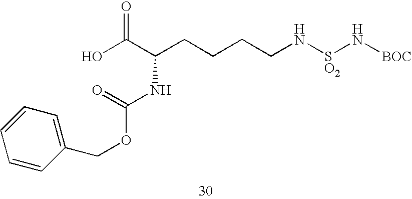 | 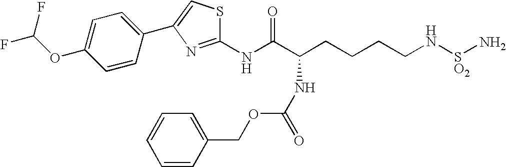 | (S)-benzyl 1-(4-(4- (difluoro-methoxy)- phenyl)- thiazol-2- ylamino)- 1-oxo-6- (sulfamoyl- amino)- hexan-2- ylcarbamate | (CD3OD) d (ppm) 1 H: 7.93 (dt, J = 8.8, 2.7 Hz, 2 H), 7.38-7.28 (m, 5 H), 7.23-7.10 (m, 3 H), 6.84 (t, J = 74 Hz, 1 H), 5.11 (d, J =6.3 Hz, 2 H), 4.34 (t, J = 5.1 Hz, 1 H), 3.03 (t, J = 6.7 Hz, 2 H), 1.94-1.69 (m, 2 H), | |
| # 1.65-1.23 (m, 4 H). LRMS (ESI): (calc.) 583.1 (found) 584.2 (MH)+ | U, B | | | | | |
| 274 | 308 | 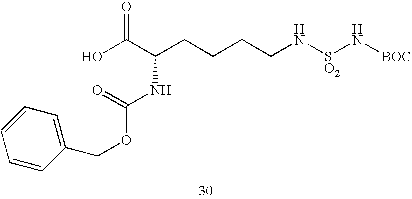 | 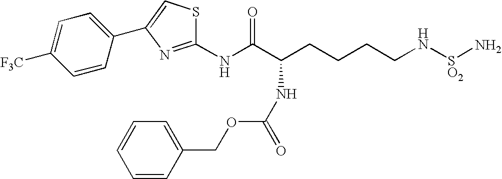 | (S)-benzyl 1-oxo-6- (sulfamoyl- amino)- 1-(4-(4- (trifluoro- methyl)- phenyl)- thiazol-2- ylamino)- hexan-2- ylcarbamate | (dmso-d6) d (ppm) 1 H: 12.48 (s, 1 H), 8.10 (d, J = 8.0 Hz, 2 H), 7.86 (s, 1 H), 7.78 (d, J = 8.4 Hz, 2 H), 7.70 (d, J =7.4 Hz, 1 H), 7.36-7.12 (m, 5 H), 6.44 (s, 2 H), 6.43 (t, J = 6.3 Hz, 1 H), 5.01 (d, J = 1.8 Hz, 2 H), | |
| # 4.25 (q, J =5.1 Hz, 1 H), 2.83 (q, J = 6.5 Hz, 2 H), 1.78-1.53 (m, 2 H), 1.52-1.23 (m, 4 H). LRMS (ESI): (calc.) 585.1 (found) 586.2 (MH)+ | U, B | | | | | |
| 275 | 309 | 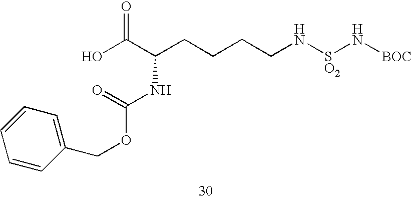 | 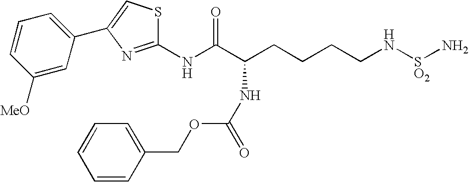 | (S)-benzyl 1-(4-(3- methoxy- phenyl)- thiazol-2- ylamino)- 1-oxo-6- (sulfamoyl- amino)- hexan-2- ylcarbamate | (dmso-d6) d (ppm) 1 H: 12.39 (s, 1 H), 7.69 (d, J = 7.2 Hz, 1 H), 7.65 (s, 1 H), 7.47-7.44 (m, 2 H), 7.36-7.11 (m, 6 H), 6.88 (dd, J = 8.2, 2.5 Hz, 1 H), 6.43 (s, 2 H), 6.43 (t, J =6.1 Hz, 1 H), 5.01 (d, J = 2.0 Hz, 2 H), 4.25 (q, J = 5.5 | |
| # Hz, 1 H), 3.78 (s, 3 H), 2.83 (q, J = 6.5 Hz, 2 H), 1.74-1.52 (m, 2 H), 1.52-1.25 (m, 4 H). LRMS (ESI): (calc.) 547.2 (found) 548.2 (MH)+ | U, B | | | | | |
| 276 | 310 | 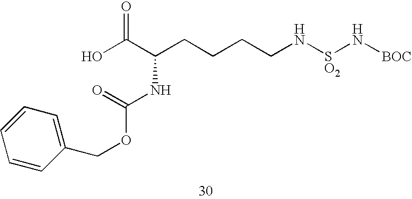 | 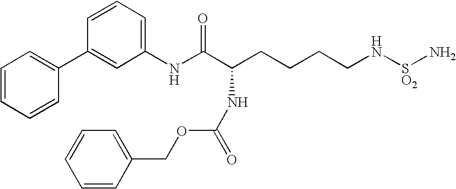 | (S)-benzyl 1- (biphenyl-3- ylamino)- 1-oxo-6- (sulfamoyl- amino)- hexan-2- ylcarbamate | (dmso-d6) d (ppm) 1 H: 10.11 (s, 1 H), 7.90 (s, 1 H), 7.62-7.56 (m, 4 H), 7.46 (t, J = 7.4 Hz, 2 H), 7.40-7.12 (m, 8 H), 6.44 (s, 2 H), 6.44 (t, J = 8.4 Hz, 1 H), 5.02 (s, 2 H), 4.12 (q, J = 5.1 Hz, 1 H), 2.83 (q, J =6.5 Hz, 2 H), 1.75-1.53 (m, 2 H), 1.52-1.24 (m, | |
| # 4 H). LRMS (ESI): (calc.) 510.2 (found) 511.3 (MH)+ | D, B | | | | | |
| 277 | 311 | 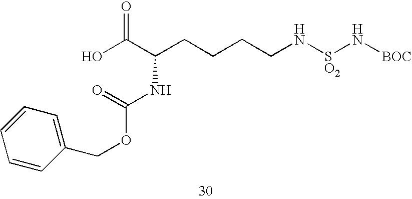 | 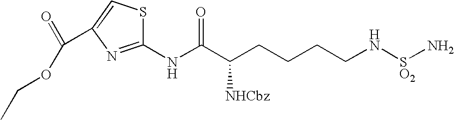 | (S)-ethyl 2-(2- (benzyloxy- carbonyl amino)-6- (sulfamoyl- amino)hex- anamido)- thiazole-4- carboxylate | (dmso-d6) d (ppm) 1 H: 12.69 (s, 1 H), 8.05 (s, 1 H), 7.69 (d, J = 7.2 Hz, 1 H), 7.38-7.11 (m, 5 H), 6.43 (s, 2 H), 5.01 (d, J = 2.2 Hz, 2 H), 4.26 (q, J = 7.0 Hz, 2 H), 4.18 (q, J =5.5 Hz, 1 H), 2.84-2.80 (m, 2 H), 1.76-1.55 (m, 2 H), 1.51-1.23 | |
| # (m, 4 H), 1.28 (t, J = 7.0 Hz, 3 H). LRMS (ESI): (calc.) 513.1 (found) 514.2 (MH)+ | U, B | | | | | |
| 278 | 312 | 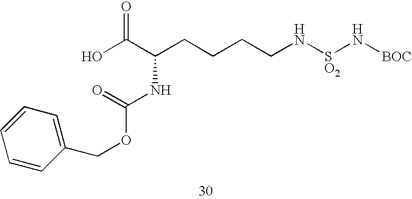 | 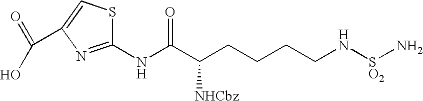 | (S)-2-(2- (benzyloxy- carbonyl amino)-6- (sulfamoyl- amino)hex- anamido)- thiazole-4- carboxylic acid | (dmso-d6) d (ppm) 1 H: 12.60 (s, 1 H), 7.97 (s, 1 H), 7.70 (d, J = 7.4 Hz, 1 H), 7.38-7.15 (m, 5 H), 6.44 (bs, 3 H), 5.01 (d, J = 2.2 Hz, 2 H), 4.19 (q, J = 5.1 Hz, 1 H), 2.82 (q, J =5.1 Hz, 2 H), 1.75-1.58 (m, 2 H), 1.55-1.27 (m, 4 H). LRMS (ESI): | |
| # (calc.) 485.1 (found) 486.2 (MH)+ | U, J, B | | | | | |
| 279 | 313 | 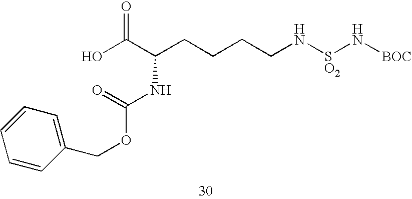 | 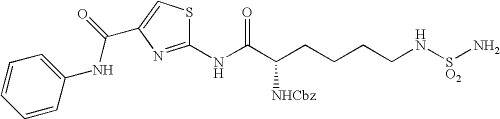 | (S)-benzyl 1-oxo-1-(4- (phenyl- carbamoyl)- thiazol-2- ylamino)-6- (sulfamoyl- amino)- hexan-2- ylcarbamate | (dmso-d6) d (ppm) 1 H: 12.51 (bs, 1 H), 9.79 (s, 1 H), 7.96 (s, 1 H), 7.75-7.72 (m, 3 H), 7.39-7.28 (m, 6 H), 7.16 (m, 6 H), 7.09 (t, J = 7.2 Hz, 1 H), 6.45-6.43 (m, 3 H), 5.02 (d, J = 2.7 Hz, 2 H), 4.27 (q, J =4.5 Hz, 1 H), 2.83 (q, J = 6.1 | |
| # Hz, 2 H), 1.75-1.58 (m, 2 H), 1.56-1.27 (m, 4 H). LRMS (ESI): (calc.) 560.2 (found) 561.3 (MH)+ | U, J, D, B | | | | | |
| 280 | 314 | 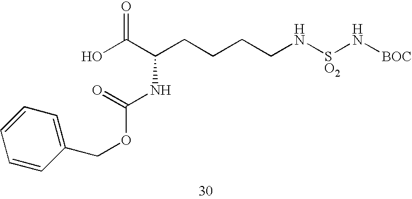 | 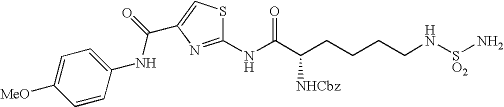 | (S)-benzyl 1-(4-(4- methoxy- phenyl- carbamoyl)- thiazol-2- ylamino)- 1-oxo-6- (sulfamoyl- amino)- hexan-2- ylcarbamate | (dmso-d6) d (ppm) 1 H: 12.47 (bs, 1 H), 9.69 (s, 1 H), 7.92 (s, 1 H), 7.72 (d, J = 6.8 Hz, 1 H), 7.65 (d, J = 9.0 Hz, 2 H), 7.38-7.28 (m, 4 H), 7.16 (bs, 1 H), 6.92 (d, J = 9.0 Hz, 1 H), 6.45-6.43 (m, 3 H), 5.02 (d, J =2.7 Hz, | |
| # 2 H), 4.27 (q, J = 4.9 Hz, 1 H), 3.73 (s, 3 H), 2.82 (q, J = 6.3 Hz, 2 H), 1.77-1.58 (m, 2 H), 1.53-1.25 (m, 4 H). LRMS (ESI): (calc.) 590.2 (found) 591.2 (MH)+ | U, J, D, B | | | | | |
| 281 | 315 | 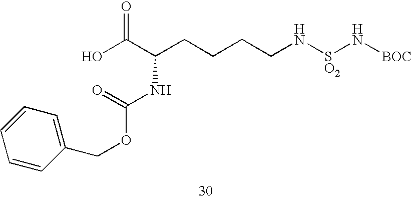 | 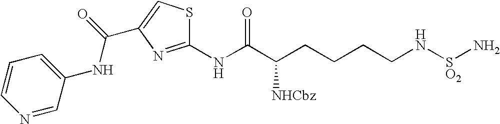 | (S)-benzyl 1-oxo-1-(4- (pyridin-3- yl- carbamoyl)- thiazol-2- ylamino)-6- (sulfamoyl- amino)- hexan-2- ylcarbamate | (dmso-d6) d (ppm) 1 H: 12.51 (bs, 1 H), 10.10 (s, 1 H), 8.90 (d, J = 1.8 Hz, 1 H), 8.30 (dd, J =4.7, 1.4 Hz, 1 H), 8.20 (d, J = 8.6 Hz, 1 H), 8.02 (s, 1 H), 7.74 (d, J = 7.4 Hz, 1 H), 7.40-7.15 (m, 6 H), 6.45-6.43 (m, 3 H), 5.02 (d, | |
| # J =2.7 Hz, 2 H), 4.29 (q, J = 4.5 Hz, 1 H), 2.83 (q, J = 6.3 Hz, 2 H), 1.77-1.57 (m, 2 H), 1.52-1.26 (m, 4 H). LRMS (ESI): (calc.) 561.1 (found) 562.2 (MH)+ | U, J, D, B | | | | | |
| 282 | 316 | 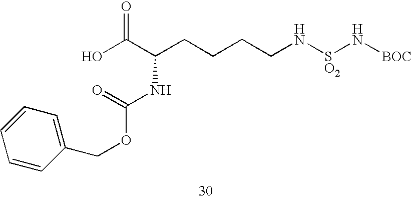 | 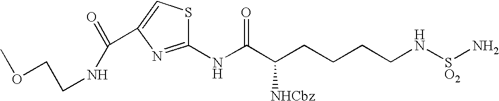 | (S)-benzyl 1-(4-(2- methoxy- ethyl- carbamoyl)- thiazol-2- ylamino)- 1-oxo-6- (sulfamoyl- amino)- hexan-2- ylcarbamate | (dmso-d6) d (ppm) 1 H: 12.61 (s, 1 H), 7.78 (s, 1 H), 7.75-7.70 (m, 1 H), 7.38-7.11 (m, 5 H), 6.44 (bs, 3 H), 5.01 (d, J = 2.2 Hz, 2 H), 4.24 (q, J = 4.7 Hz, 1 H), 3.45-3.40 (m, 4 H), 3.26 (s, 3 H), 2.82 (q, J = 6.1 Hz, 2 H), | |
| # 1.72-1.54 (m, 2 H), 1.52-1.24 (m, 4 H). LRMS (ESI): (calc.) 542.2 (found) 543.2 (MH)+ | U, J, D, B | | | | | |
| 283 | 317 | 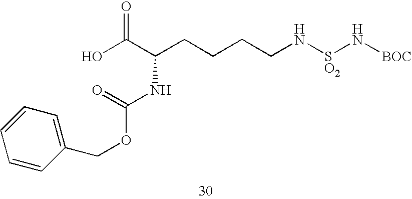 | 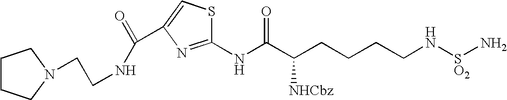 | (S)-benzyl 1-oxo-1- (4-(2- (pyrrolidin- 1-yl)ethyl- carbamoyl)- thiazol-2- ylamino)-6- (sulfamoyl- amino)- hexan-2- ylcarbamate | (dmso-d6) d (ppm) 1 H: 12.32 (s, 1 H), 9.48 (bs, 1 H), 8.24 (t, J = 6.1 Hz, 1 H), 7.84 (s, 1 H), 7.76 (d, J = 7.2 Hz, 1 H), 7.38-7.14 m, 5 H), 6.44 (m, 3 H), 5.01 (d, J = 2.7 Hz, 2 H), 4.29 (q, J =5.5 Hz, 1 H), | |
| #3.66-3.56 (m, 4 H), 3.08-2.97 (m, 2 H), 2.82 (q, J = 6.3 Hz, 2 H), 2.08-1.93 (m, 2 H), 1.89-1.78 m, 2 H), 1.70-1.56 (m, 2 H), 1.52-1.24 (m, 4 H). LRMS (ESI): (calc.) 581.2 (found) 582.3 (MH)+ | U, J, D, B | | | | | |
| 284 | 318 | 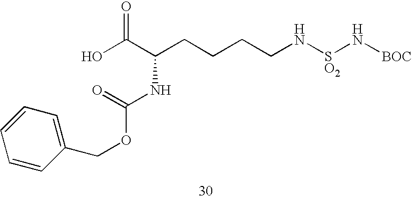 | 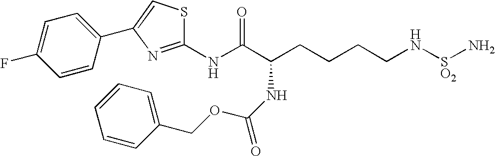 | (S)-benzyl 1-(4-(4- fluoro- phenyl)- thiazol-2- ylamino)- 1-oxo-6- (sulfamoyl- amino)- hexan-2- ylcarbamate | (dmso-d6) d (ppm) 1 H: 12.39 9 s, 1 H), 7.93 (dd, J =9.0, 5.7 Hz, 2 H), 7.69 (d, J = 7.2 Hz, 1 H), 7.61 (s, 1 H), 7.38-7.12 (m, 7 H), 6.44 (bs, 3 H), 5.01 (d, J =2.0 Hz, 2 H), 4.25 (q, J = 4.7 Hz, 1 H), 2.84-2.79 (m, 2 H), 1.76-1.56 (m, | |
| # 2 H), 1.53-1.22 (m, 4 H). LRMS (ESI): (calc.) 535.1 (found) 536.1 (MH)+ | U, B | | | | | |
| 285 | 319 | 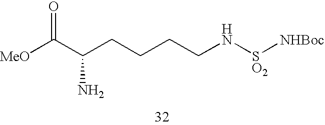 | 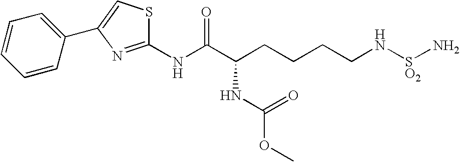 | (S)-methyl 1-oxo-1-(4- phenyl- thiazol-2- ylamino)-6- (sulfamoyl- amino)- hexan-2- ylcarbamate | (dmso-d6) d (ppm) 1 H: 12.37 (s, 1 H), 7.88 (dd, J = 8.4, 1.4 Hz, 2 H), 7.62 (s, 1 H), 7.55 (d, J =7.2 Hz, 1 H), 7.42 (t, J = 7.8 Hz, 2 H), 7.31 (t, J = 7.2 Hz, 1 H), 6.44 (s, 2 H), 6.43 (t, J = 6.3 Hz, 1 H), 4.23 (q, J =4.5 Hz, 2 H), 3.53 (s, | |
| # 3 H), 2.83 (q, J =6.3 Hz, 2 H), 1.77-1.57 (m, 2 H), 1.56-1.24 (m, 6 H). LRMS (ESI): (calc.) 441.1 (found) 442.2 (MH)+ | AA, J, D, B | | | | | |
| 291 | 336 | 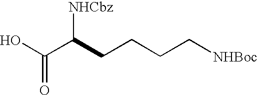 | 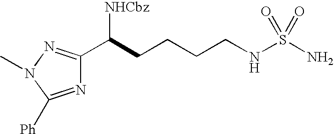 | (S)-benzyl 1-(1-methyl- 5-phenyl- 1H-1,2,4- triazol- 3-yl)-5- (sulfamoyl- amino) pentyl- carbamate | (DMSO-d₆) δ (ppm) ¹H: 8.01 (d, J = 8.0 Hz, 1 H), 7.93 (d, J = 6.8 Hz, 2 H), 7.45-7.39 (m, 3 H), 7.38-7.20 (m, should be 5 H but broadening gives only 4 H), 6.44-6.42 (m, 3 H), 5.04-4.97 (m, 2 H), 4.80 (q, J = 8.0 Hz, 1 H), 3.89 (s, | |
| # 3 H), 2.82 (q, J = 6.8 Hz, 2 H), 1.89-1.83 (m, 2 H), 1.49-1.32 (m, 3 H), 1.30-1.18 (m, 1 H). LRMS (ESI): (calc) 4725.2 (found) 473.3 (MH)⁻. | JJ, KK, E, M, B | | | | | |
| 295 | 346 | 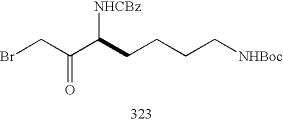 | 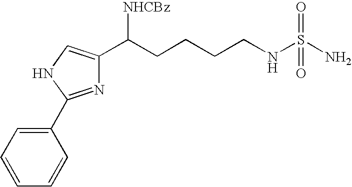 | (S)-benzyl 1-(2- phenyl-1H- imidazol- 4-yl)-5- (sulfamoyl- amino)- pentyl- carbamate | (CD₃OD) δ (ppm): 7.82 (d, J = 7.2 Hz, 2 H), 7.44 (t, J =7.2 Hz, 1 H), 7.39-7.26 (m, 6 H), 6.98 (s, 1 H), 5.13-5.07 (m, 2 H), 4.734 (t, J = 6.81 H), 3.02 (t, J = 6.8 Hz, 2 H), 2.00-1.87 (m, 1 H), 1.87-1.75 (m, 1 H), 1.67-1.55 (m, 2 H), 1.55-1.37 (m, | |
| # 2 H). LRMS: 457.18 (calc) 458.2 (found) (MH)+ | GG, B, M, E | | | | | |
| 296 | 347 | 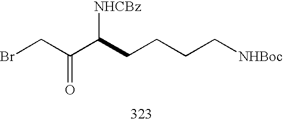 | 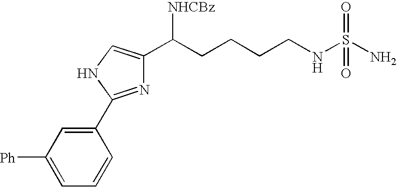 | (S)-benzyl 1-(2- (biphenyl- 3-yl)-1H- imidazol- 5-yl)-5- (sulfamoyl- amino)- pentyl- carbamate | (CD₃OD) δ (ppm): 8.14 (s, 1 H), 7.80 (d, J = 7.6 Hz, 1 H), 7.69 (d, J = 6.8 Hz, 2 H), 7.63 (d, J =8.0 Hz, 1 H), 7.52 (t, J = 7.6 Hz, 1 H), 7.46 (t, J = 7.6 HZ, 2 H), 7.38-7.25 (m, 7 H), 7.01 (s, 1 H), 5.13-5.06 (m, 2 H), 4.77-4.74 (m, 1 | |
| # H), 3.02 (t, J =7.2 Hz, 2 H), 2.00-1.90 (m, 1 H), 1.88-1.76 (m, 1 H), 1.66-1.56 (m, 2 H), 1.54-1.38 (m, 2 H). ). LRMS: 533.21 (calc) 534.3 (found) (MH)+ | GG, B, M, E | | | | | |
| 297 | 348 | 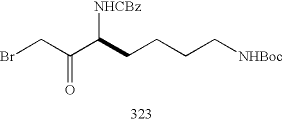 | 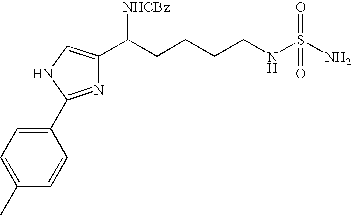 | (S)-benzyl 5- (sulfamoyl- amino)- 1-(2-p-tolyl- 1H- imidazol-4- yl)pentyl- carbamate | (CD₃OD) δ (ppm): 7.71 (d, J = 8.4 Hz, 2 H), 7.35-7.24 (m, 4 H), 7.23 (d, J =7.6 Hz, 2 H), 6.95 (s, 1 H), 5.10 (d, J =12.4 Hz, 1 H), 5.08 (d, J = 12.8 Hz, 2 H), 4.72 (m, 1 H), 3.01 (t, J = 7.2 Hz, 2 H), 1.98-1.88 (m, 1 H), 1.86-1.74 (m, 1 H), 1.66-1.54 | |
| # (m, 2 H), 1.52-1.38 (m, 2 H). LRMS: 471.2 (calc) 472.2 (found) (MH)+ | GG, B, M, E | | | | | |
| 298 | 349 | 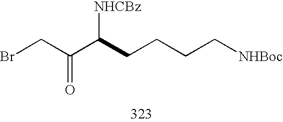 | 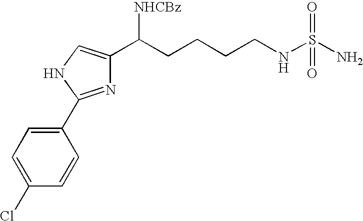 | (S)-benzyl 1-(2-(4- chloro- phenyl)-1H- imidazol- 4-yl)-5- (sulfamoyl- amino)- pentyl- carbamate | (CD₃OD) δ (ppm): 7.81 (d, J = 8.8 Hz, 2 H), 7.44 (d, J =8.8 Hz, 2 H), 7.35-7.27 (m, 5 H),, 7.00 (s, 1 H), 5.10 (d, J = 12.8 Hz, 1 H), 5.08 (d, J =12.8 Hz, 2 H), 4.73 (m, 1 H), 3.01 (t, J = 6.8 Hz, 2 H), 1.98-1.88 (m, 1 H), 1.86-1.74 | |
| # (m, 1 H), 1.66-1.56 (m, 2 H), 1.52-1.38 (m, 2 H). LRMS: 491.1 (calc) 492.2 (found) (MH)+ | GG, B, M, E | | | | | |
| 299 | 350 | 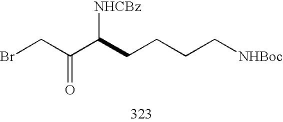 | 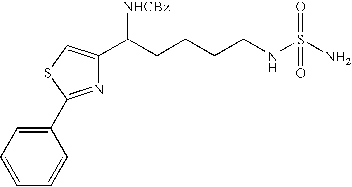 | (S)-benzyl 1-(2- phenyl- thiazol- 4-yl)-5- (sulfamoyl- amino)- pentyl- carbamate | (DMSO-d₆) δ(ppm): 7.92-7.89 (m, 2 H), 7.79 (d, J = 8.8 Hz, 1 H), 7.51-7.46 (m, 3 H), 7.39 (s, 1 H), 7.36-7.20 (m, 5 H), 6.44 (m, 2 H), 5.05 (s, 2 H), 4.75-4.69 (m, 1 H), 2.83 (q, J = 6.8 Hz, 2 H), 1.90-1.81 (m, 1 H), 1.75-1.65 (m, 1 H), 1.50-1.44 (m, 2 H), 1.42-1.37 | |
| # (m, 2 H). LRMS: 474.14 (calc) 475.1 (found) (MH)+ | HH, B, M, B | | | | | |
| 300 | 351 | 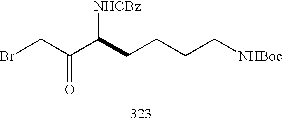 | 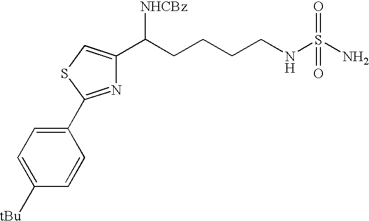 | (S)-benzyl 1-(2-(4-tert- butyl- phenyl)- thiazol- 4-yl)-5- (sulfamoyl- amino)- pentyl- carbamate | (DMSO-d₆) δ(ppm): 7.82 (d, J =8.0 Hz, 2 H), 7.78 (d, overlapping signal, 1 H), 7.50 (d, J = 8.0 Hz, 1 H), 7.36-7.20 (m, 6 H), 6.44 (m, 2 H), 5.03 (s, 2 H), 4.75-4.68 (m, 1 H), 2.83 (m, 2 H), 1.90-1.81 (m, 1 H), 1.75-1.65 (m, 1 H), 1.50-1.44 (m, 2 H), | |
| # 1.42-1.35 (m, 2 H), 1.30 (s, 9 H). LRMS: 530.20 (calc) 531.1 (found) (MH)+ | HH, B, M, B | | | | | |
| 301 | 352 | 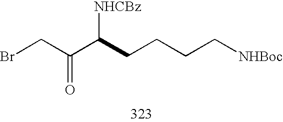 | 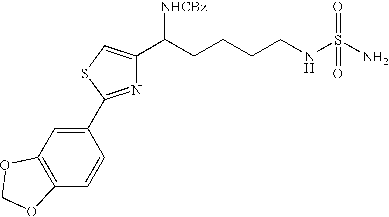 | (S)-benzyl 1-(2-(benzo- [d][1,3]- dioxol-5- yl)thiazol- 4-yl)-5- (sulfamoyl- amino)- pentyl- carbamate | (DMSO-d₆) δ(ppm): 7.76 (d, J =8.8 Hz, 1 H), 7.44-7.42 (m, 2 H), 7.92-7.89 (m, 2 H), 7.36-7.20 (m, 5 H), 7.30 (s, 1 H), 7.00 (d, J = 8.4 Hz, 1 H), 6.44 (s, 2 H), 6.10 (s, 2 H), 6.44 (m, 2 H), 5.03 (s, 2 H), 4.73-4.55 (m, 1 H), 2.82 (q, J | |
| # = 6.8 Hz, 2 H), 1.91-1.78 (m, 1 H), 1.75-1.62 (m, 1 H), 1.50-1.40 (m, 2 H), 1.42-1.35 (m, 2 H). LRMS: 518.13 (calc) 519.2 (found) (MH)+ | HH, B, M, B | | | | | |
| 302 | 353 | 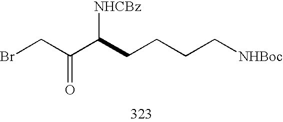 | 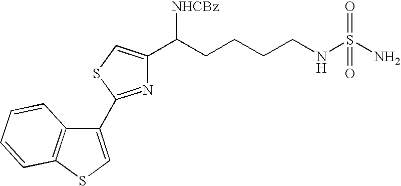 | (S)-benzyl 1-(2-(benzo- [b]thiophen- 3-yl)thiazol- 4-yl)-5- (sulfamoyl- amino)- pentyl- carbamate | (DMSO-d₆) δ(ppm): 8.76 (d, J =8.0 Hz, 1 H), 8.41 (s, 1 H), 8.08 (d, J =8.0 Hz, 1 H), 7.53 (t, J = 7.2 Hz, 1 H), 7.47 (t, J = 7.6 Hz, 1 H), 7.41 (s, 1 H), 7.35-7.20 (m, 5 H), 6.44 (s, 3 H), 5.04 (s, 2 H), 4.82-4.76 (m, 1 H), 2.85 (s, J = 6.0 | |
| # Hz, 2 H), 2.00-1.86 (m, 1 H), 1.84-1.72 (m, 1 H), 1.56-1.46 (m, 2 H), 1.44-1.30 (m, 2 H). LRMS: 530.11 (calc) 531.2 (found) (MH)+ | HH, B, M, B | | | | | |
| 303 | 354 | 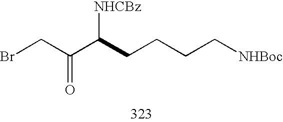 | 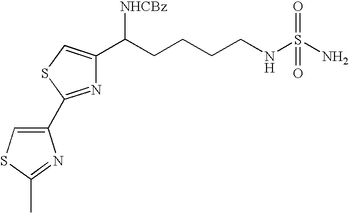 | S)-benzyl 1-(2′-methyl- 2,4′- bithiazol- 4-yl)-5- (sulfamoyl- amino)- pentyl- carbamate | (DMSO-d₆) δ(ppm): 8.01 (s, 1 H), 7.79 (d, J =9.2 Hz, 1 H), 7.40-7.20 (m, 6 H), 6.44 (m, 2 H), 5.06-4.99 (s, 2 H), 4.73-4.68 (m, 1 H), 2.82 (q, J = 8.0 Hz, 2 H), 2.70 (s, 3 H), 1.90-1.78 (m, 1 H), 1.76-1.60 (m, 1 H), 1.52-1.40 (m, 2 H), 1.40-1.24 (m, 2 | |
| # H). LRMS: 495.11 (calc) 496.2 (found) (MH)+ | HH, B, M, B | | | | | |
| 304 | 355 | 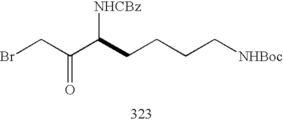 | 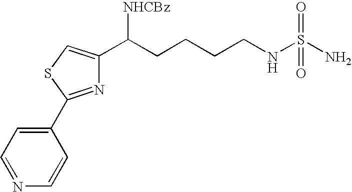 | (S)-benzyl 1-(2- (pyridin-4- yl)thiazol- 4-yl)-5- (sulfamoyl- amino)- pentyl- carbamate | (DMSO-d₆) δ(ppm): 8.68 (d, J =6.0 Hz, 2 H), 7.85 (d, J = 6.0 Hz, 2 H), 7.83 (d, overlapping signal, 1 H), 7.58 (s, 1 H), 7.35-7.29 (m, 5 H), 6.44 (m, 2 H), 5.03 (s, 2 H), 4.78-4.72 (m, 1 H), 2.8. (q, J = 6.0 Hz, 2 H), 1.90-1.78 (m, 1 H), 1.78-1.67 (m, 1 | |
| # H), 1.52-1.40 (m, 2 H), 1.40-1.24 (m, 2 H). LRMS: 475.13 (calc) 476.2 (found) (MH)+ | HH, B, M, B | | | | | |
| 305 | 356 | 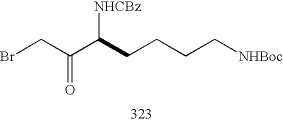 | 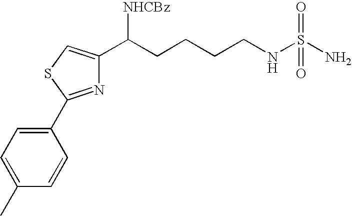 | (S)-benzyl 5- (sulfamoyl- amino)- 1-(2-p- tolylthiazol- 4-yl)pentyl- carbamate | (DMSO-d6) δ(ppm): 7.79 (d, J =8.0 Hz, 2 H), 7.78 (d, J = 8.8 Hz, 1 H), 7.35-7.26 (m, 6 H), 7.28 (d, J =8.4 Hz, 2 H), 6.44-6.41 (m, 3 H), 5.03 (s, 2 H), 4.70 (dt, J = 5.2, 8.8, 1 H), 2.82 (q, J =6.4 Hz, 2 H), 2.34 (s, 3 H), 1.89-1.82 (m, 1 H), 1.74-1.67 (m, 1 H), | |
| # 1.52-1.42 (m, 2 H), 1.39-1.24 (m, 2 H). LRMS: 488.2 (calc) 489.2 (found) (MH)+ | HH, B, M, B | | | | | |
| 306 | 357 | 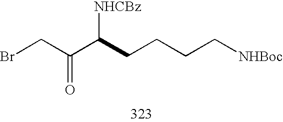 | 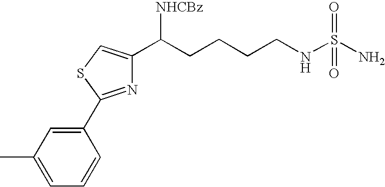 | (S)-benzyl 5- (sulfamoyl- amino)- 1-(2-m- tolylthiazol- 4-yl)pentyl- carbamate | (DMSO-d6) δ(ppm): 7.79 (d, J =8.8 Hz, 1 H), 7.73 (s, 1 H), 7.69 (d, J =7.6 Hz, 1 H), 7.38-7.32 (m, 5), 7.31-7.26 (m, 2 H), 6.44-6.41 (m, 3 H), 5.03 (s, 2 H), 4.71 (dt, J = 5.2, 8.8, 1 H), 2.83 (q, J =7.2 Hz, 2 H), 2.37 (s, 3 H), 1.89-1.82 (m, 1 H), 1.74-1.67 (m, 1 H), | |
| # 1.52-1.42 (m, 2 H), 1.39-1.24 (m, 2 H). LRMS: 488.2 (calc) 489.2 (found) (MH)+ | HH, B, M, B | | | | | |
| 307 | 358 | 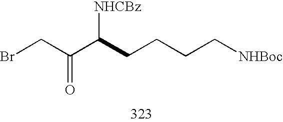 | 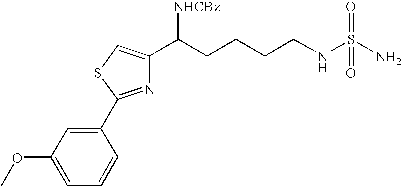 | (S)-benzyl 1-(2-(3- methoxy- phenyl)- thiazol- 4-yl)-5- (sulfamoyl- amino)- pentyl- carbamate | (DMSO-d6) δ(ppm): 7.80 (d, J =8.8 Hz, 1 H), 7.46 (d, J = 8.0 Hz, 1 H), 7.42-7.41 (m, 1 H), 7.39 (s, 1 H), 7.38-7.28 (m, 5 H), 7.04 (ddd, J = 1.2, 2.8, 8.0 Hz, 1 H), 6.44-6.41 (m, 3 H), 5.03 (s, 2 H), 4.72 (dt, J = 5.2, 8.8, 1 H), 3.81 (s, 3 H), 2.83 | |
| # (q, J = 6.4 Hz, 2 H), 1.89-1.82 (m, 1 H), 1.73-1.67 (m, 1 H), 1.50-1.44 (m, 2 H), 1.39-1.24 (m, 2 H). LRMS: 504.2 (calc) 505.2 (found) (MH)+ | HH, B, M, B | | | | | |
| 308 | 359 | 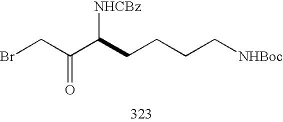 | 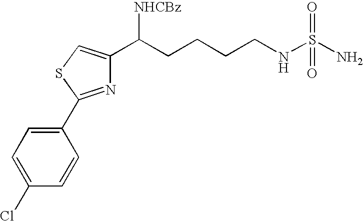 | (S)-benzyl 1-(2-(4- chloro- phenyl)- thiazol- 4-yl)-5- (sulfamoyl- amino)- pentyl- carbamate | (DMSO-d6) δ(ppm): 7.92 (d, J =8.4 Hz, 2 H), 7.80 (d, J = 8.8 Hz, 2 H), 7.54 (d, J = 8.8 Hz, 1 H), 7.42 (s, 1 H), 7.35-7.26 (m, 5 H), 6.44-6.41 (m, 3 H), 5.03 (s, 2 H), 4.71 (dt, J = 5.2, 8.4, 1 H), 2.82 (q, J =6.4 Hz, 2 H), 1.89-1.80 (m, 1 H), | |
| #1.74-1.67 (m, 1 H), 1.52-1.42 (m, 2 H), 1.40-1.24 (m, 2 H). LRMS: 508.1 (calc) 509.1 (found) (MH)+ | HH, B, M, B | | | | | |
| 309 | 360 | 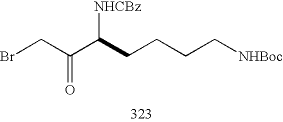 | 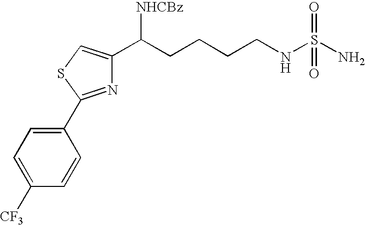 | (S)-benzyl 5- (sulfamoyl- amino)- 1-(2-(4- (trifluoro- methyl)- phenyl)- thiazol-4- yl)pentyl- carbamate | (DMSO-d6) δ(ppm): 8.12 (d, J =8.0 Hz, 2 H), 7.84 (d, J = 8.0 Hz, 2 H), 7.83 (d, J = 8.8 Hz, 1 H), 7.52 (s, 1 H), 7.35-7.26 (m, 5 H), 6.44-6.41 (m, 3 H), 5.03 (m, 2 H), 4.75 (dt, J = 5.6, 8.8, 1 H), 2.82 (q, J =7.2 Hz, 2 H), 1.92-1.80 | |
| # (m, 1 H), 1.76-1.67 (m, 1 H), 1.52-1.42 (m, 2 H)+NL, 1.40-1.24 (m, 2 H). LRMS: 542.2 (calc) 543.1 (found) | HH, B, M, B | | | | | |
| 310 | 361 | 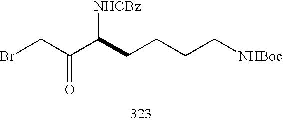 | 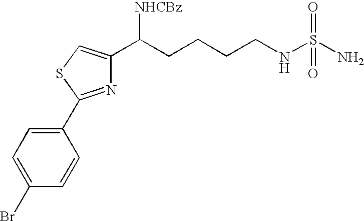 | (S)-benzyl 1-(2-(4- bromo- phenyl)- thiazol- 4-yl)-5- (sulfamoyl- amino)- pentyl- carbamate | (DMSO-d6) δ(ppm): 7.85 (d, J =8.8 Hz, 2 H), 7.80 (d, J = 8.4 Hz, 1 H), 7.68 (d, J = 8.8 Hz, 2 H), 7.42 (s, 1 H), 7.35-7.26 (m, 5 H), 6.44-6.41 (m, 3 H), 5.03 (m, 2 H), 4.72 (dt, J = 5.2, 8.4, 1 H), 2.82 (q, J =6.4 Hz, 2 H), 1.89-1.80 (m, 1 H), | |
| #1.75-1.65 (m, 1 H), 1.51-1.43 (m, 2 H), 1.40-1.24 (m, 2 H). LRMS: 552.05 (calc) 553.1 (found) (MH)+ | HH, B, M, B | | | | | |
| 311 | 362 | 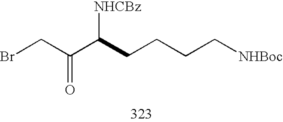 | 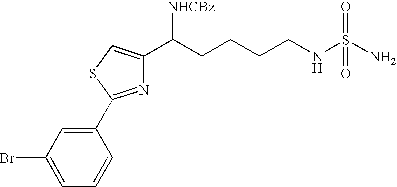 | (S)-benzyl 1-(2-(3- bromo- phenyl)- thiazol- 4-yl)-5- (sulfamoyl- amino)- pentyl- carbamate | (DMSO-d6) δ(ppm): 8.08 (s, 1 H), 7.89 (d, J =8.4 Hz, 1 H), 7.81 (d, J = 8.8 Hz, 1 H), 7.66 (d, J = 8.0 Hz, 1 H), 7.85 (d, J =8.8 Hz, 2 H), 7.46 (s, 1 H), 7.43 (d, J =7.6 Hz, 1 H), 7.35-7.26 (m, 5 H), 6.44-6.41 (m, 3 H), 5.03 (s, 2 H), 4.72 (dt, | |
| # J = 5.2, 8.8, 1 H), 2.82 (q, J =6.8 Hz, 2 H), 1.90-1.80 (m, 1 H), 1.74-1.67 (m, 1 H), 1.51-1.43 (m, 2 H), 1.40-1.24 (m, 2 H). LRMS: 552.05 (calc) 553.0 (found) (MH)+ | HH, B, M, B | | | | | |
| 312 | 363 | 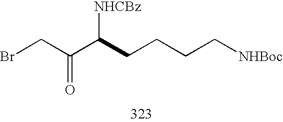 | 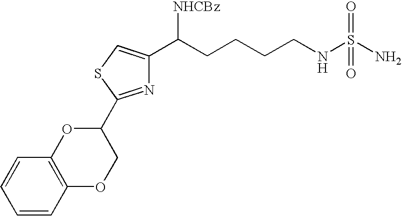 | benzyl (1S)- 1-(2-(2,3- dihydro- benzo[b]- [1,4]dioxin- 2-yl)thiazol- 4-yl)-5- (sulfamoyl- amino)- pentyl- carbamate | (DMSO-d6) δ(ppm): 7.77 (t, J =9.2 Hz, 1 H), 7.40-7.29 (m, 6 H), 7.01-6.98 (m, 1 H), 6.92-6.86 (m, 3 H), 6.44-6.41 (m, 3 H), 5.68 (dd, J = 2.8, 6.0 Hz, 1 H), 5.05-4.98 (m, 2 H), 4.69 (dt, J = 5.2, 8.8 Hz, 1 H), 4.52 (d, | |
| # J = 11.6 Hz, 1 H), 4.34 (dd, J =6.0, 12.0, 1 H), 2.81 (q, J = 6.8 Hz, 2 H), 1.84-1.72 (m, 1 H), 1.68-1.60 (m, 1 H), 1.48-1.40 (m, 2 H), 1.38-1.22 (m, 2 H). LRMS: 532.1 (calc) 533.2 (found) (MH)+ | HH, B, M, B | | | | | |
| 313 | 364 | 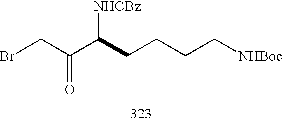 | 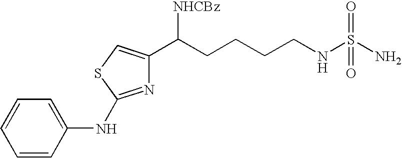 | (S)-benzyl 1-(2-(phenyl- amino)- thiazol- 4-yl)-5- (sulfamoyl- amino)- pentyl- carbamate | (DMSO-d6) δ(ppm): 10.12 (s, 1 H), 7.63 (d, J =8.8 Hz, 1 H), 7.59 (d, J = 9.6 Hz, 2 H), 7.35-7.25 (m, 7 H), 6.90 (t, J = 7.2 Hz, 1 H), 6.502 (s, 1 H), 6.44-6.41 (m, 3 H), 5.03 (m, 2 H), 4.50 (dt, J = 5.6, 8.8 Hz, 1 H), 2.82 (q, J =6.8 Hz, 2 H), 1.88-1.78 | |
| # (m, 1 H), 1.68-1.58 (m, 1 H), 1.52-1.42 (m, 2 H), 1.40-1.26 (m, 2 H). LRMS: 489.2 (calc) 490.1 (found) (MH)+ | HH, B, M, B | | | | | |
| 314 | 365 | 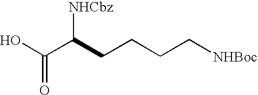 | 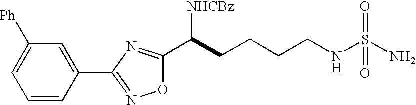 | (S)-benzyl 1-(3- (biphenyl- 3-yl)-1,2,4- oxadiazol-5- yl)-5- (sulfamoyl- amino)- pentyl- carbamate | (DMSO-d6) δ(ppm): 8.25 (d, J =7.6 Hz, 1 H), 8.19 (s, 2 H), 7.98 (d, J =7.6 Hz, 1 H), 7.90-7.87 (m, 1 H), 7.70 (d, J = 7.6 Hz, 2 H), 7.67 (t, J =7.6 Hz, 1 H), 7.51 (t, J = 7.2 Hz, 2 H), 7.44-7.40 (m, 1 H), 7.36-7.16 (m, 5 H), 6.46 (m, 3 H), | |
| # 5.07 (d, J = 12.8, 1 H), 5.06 (d, J = 12.8 Hz, 1 H), 4.97-4.91 (m, 1 H), 2.84 (q, J = 6.4 Hz, 2 H), 2.00-1.80 (m, 2 H), 1.55-1.30 (m, 4 H). LRMS: 535.13 (calc) 536.3 (found) (MH)+ | MM, B, M, B | | | | | |
| 315 | 366 | 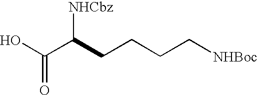 | 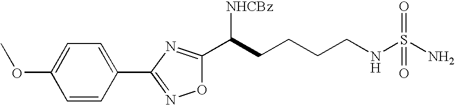 | (S)-benzyl 1-(5-(4- methoxy- phenyl)- 1,2,4- oxadiazol-3- yl)-5- (sulfamoyl- amino)- pentyl- carbamate | (CD₃OD) δ (ppm) 1 H: 7.97 (d, 2 H, J = 8.8 Hz), 7.29-7.38 (m, 4 H), 7.18-7.21 (m, 1 H), 7.04 (d, 2 H, J =8.9 Hz), 5.12 (s, 2 H), 4.98-5.02 (m, 1 H), 3.86 (s, 3 H), 3.03 (t, 2 H, J =6.6 Hz), 1.91-2.10 (m, 2 H), 1.49-1.65 (m, 4 H). LRMS: | |
| # 489.17 (calc) 490.2 (found) (MH)+ | LL, B, M, B | | | | | |
| 316 | 367 | 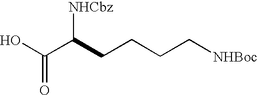 | 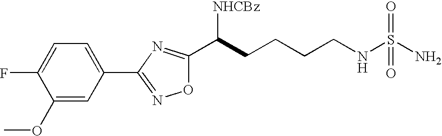 | (S)-benzyl 1-(5-(4- fluoro-3- methoxy- phenyl)- 1,2,4- oxadiazol-3- yl)-5- (sulfamoyl- amino)- pentyl- carbamate | (CD₃OD) δ (ppm) 1 H: 7.73 (d, 1 H, J = 8.4 Hz), 7.63-7.65 (m, 1 H), 7.29-7.38 (m, 4 H), 7.18-7.25 (m, 2 H), 5.12 (s, 2 H), 4.93-5.04 (m, 1 H), 3.94 (s, 3 H), 3.03 (t, 2 H, J = 6.6 Hz), 1.88-2.07 (m, 2 H), 1.51-1.62 (m, 4 H). LRMS: | |
| # 507.16 (calc) 508.2 (found) (MH)+ | LL, B, M, B | | | | | |
| 317 | 368 | 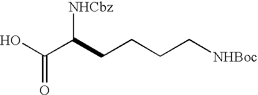 | 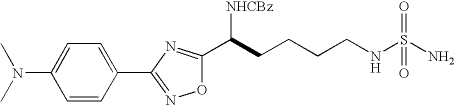 | (S)-benzyl 1-(5-(4- (dimethyl- amino)- phenyl)- 1,2,4- oxadiazol-3- yl)-5- (sulfamoyl- amino)- pentyl- carbamate | (CD₃OD) δ (ppm) 1 H: 7.85 (d, 2 H, J = 8.8 Hz), 7.28-7.38 (m, 4 H), 7.19-7.21 (m, 1 H), 6.80 (d, 2 H, J = 9.1 Hz), 5.12 (s, 2 H), 4.96-5.00 (m, 1 H), 3.01-3.05 (m, 8 H), 1.93-2.01 (m, 2 H), 1.50-1.60 (m, 4 H). LRMS: 502.2 (calc) | |
| #503.3 (found) (MH)+ | LL, B, M, B | | | | | |
| 318 | 369 | 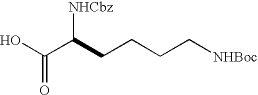 | 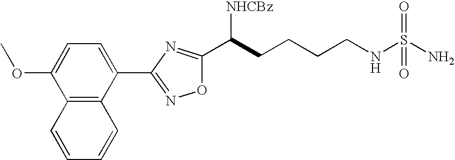 | (S)-benzyl 1-(3-(4- methoxy- naphthalen- 1-yl)-1,2,4- oxadiazol-5- yl)-5- (sulfamoyl- amino)- pentyl- carbamate | (DMSO-d6) δ(ppm) 1 H: 8.82 (d, 1 H, J = 8.6 Hz), 8.20-8.30 (m, 3 H), 7.70-7.72 (m, 1 H), 7.68-7.69 (m, 1 H), 7.30-7.37 (m, 4 H), 7.15 (d, 1 H, 8.4 Hz), 6.45-6.48 (m, 3 H), 5.07 (s, 2 H), 4.93-4.97 (m, 1 H), 4.05 (s, 3 H), 2.85 (q, 2 H, J =6.4 | |
| # Hz), 1.89-2.02 (m, 2 H), 1.37-1.54 (m, 4 H). LRMS: 539.18 (calc) 540.2 (found) (MH)+ | MM, B, M, B | | | | | |
| 319 | 370 | 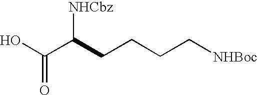 | 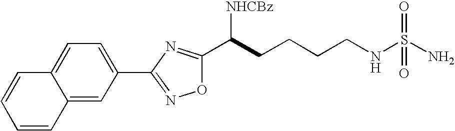 | (S)-benzyl 1-(3- (naphthalen- 2-yl)-1,2,4- oxadiazol-5- yl)-5- (sulfamoyl- amino)- pentyl- carbamate | (DMSO-d6) δ(ppm) 1 H: 8.62 (s, 1 H), 8.25 (d, 1 H, J = 7.2 Hz), 8.00-8.14 (m, 4 H), 7.59-7.66 (m, 2 H), 7.28-7.37 (m, 4 H), 6.46-6.48 (m, 3 H), 5.07 (s, 2 H), 4.95 (q, 1 H, J = 8.4 Hz), 2.83-2.86 (m, 2 H), 1.88-1.96 (m, 2 H), 1.37-1.50 (m, 4 H). LRMS: | |
| # 507.17 (calc) 510.2 (found) (MH)+ | MM, B, M, B | | | | | |
| 320 | 371 | 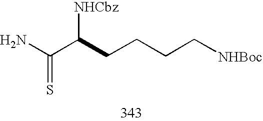 | 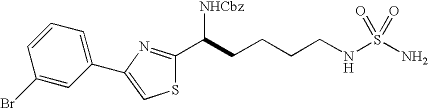 | (S)-benzyl 1-(4-(3- bromo- phenyl)- thiazol- 2-yl)-5- (sulfamoyl- amino)- pentyl- carbamate | (DMSO-d6) δ(ppm) 1 H: 8.28 (d, 1 H, J = 8.0 Hz), 8.19-8.21 (m, 2 H), 8.02 (dt, 1 H, J =7.8 Hz, 1.5 Hz), 7.59-7.62 (m, 1 H), 7.36-7.50 (m, 5 H), 7.28-7.31 (m, 1 H), 7.21-7.24 (m, 1 H), 6.52-6.55 (m, 3 H), 5.15 (q, 2 H, J = 7.0 Hz), 4.89-4.95 (m, 1 H), | |
| # 2.90-2.96 (m, 2 H), 2.00-2.10 (m, 1 H), 1.8-1.92 (m, 1 H), 1.45-1.65 (m, 4 H). LRMS: (calc) 552.05 (found) 553.1 (MH)+ | HH, B, M, B | | | | | |
| 321 | 372 | 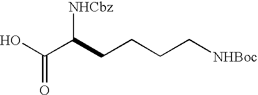 | 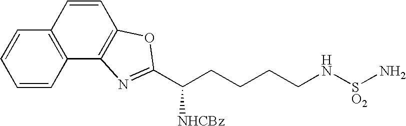 | (S)-benzyl 1- (naphtho- [1,2-d]- oxazol- 2-yl)-5- (sulfamoyl- amino)- pentyl- carbamate | (CD₃OD) δ (ppm) 1 H: 8.43 (d, 1 H, J = 8.2 Hz), 8.01 (d, 1 H, J = 8.2 Hz), 7.88 (d, 1 H, J =8.8 Hz), 7.75 (d, 1 H, J = 8.9 Hz), 7.66 (t, 1 H, J =8.0 Hz), 7.55 (t, 1 H, J = 8.0 Hz), 7.27-7.39 (m, 4 H), 5.07-5.16 (m, 3 H), 3.02-3.05 (m, 2 | |
| # H), 2.01-2.08 (m, 1 H), 2.12-2.21 (m, 1 H), 1.48-1.65 (m, 4 H). | A, Q, M, B | | | | | |
| 322 | 373 | 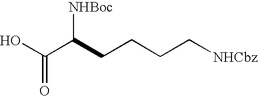 | 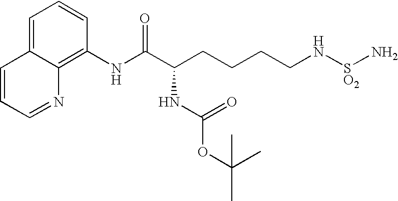 | (S)-tert- butyl 1- oxo-1- (quinolin-8- ylamino)-6- (sulfamoyl- amino)- hexan-2- ylcarbamate | (DMSO-d6) δ(ppm) 1 H: 10.48 (s, 1 H), 8.86-8.88 (m, 1 H), 8.62-8.65 (d, 1 H, J = 7.6 Hz), 8.42 (d, 1 H, J = 8.4 Hz), 7.55-7.69 (m, 4 H), 4.03-4.06 (m, 1 H), 2.84 (q, 2 H, J1 = 13 Hz, J2 =6.8 Hz), 1.81 (m, 1 H), 1.63-1.67 (m, 1 H), 1.38-1.48 (m, 13 | |
| # H), 1.13-1.17 (m, 1 H). LRMS: (calc) 451.19 (found) 452.3 (MH)+ | U, C, O, C | | | | | |
| 324 | 379 | | 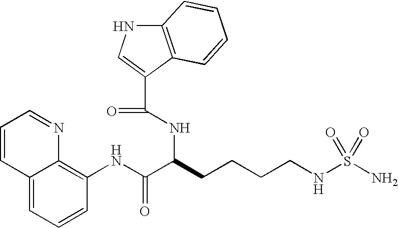 | (S)-N-(1- oxo-1- (quinolin-8- ylamino)-6- (sulfamoyl- amino)- hexan-2- yl)-1H- indole-3- carboxamide | (CD3OD) d (ppm) 1 H: 8.69-8.66 (m, 2 H), 8.27 (d, 1 H, J = 8.4 Hz), 8.16 (s, 1 H), 8.14 (d, 1 H, J = 7.6 Hz), 7.62 (d, 1 H, J =7.2 Hz), 7.55 (t, 1 H, J = 8.0 Hz), 7.50-7.45 (m, 2 H), 7.21 (t, 1 H, J =6.8 Hz), 7.16 (t, 1 H, J = 7.2 Hz), 4.84 | |
| # (q, 1 H, J =4.4 Hz), 3.08 (t, 2 H, J = 6.4 Hz), 2.21-2.18 (m, 1 H), 2.10-1.97 (m, 1 H), 1.73-1.62 (m, 4 H). LRMS (ESI): (calc.) 494.2 (found) 495.4 (MH)+ | A, B | | | | | |

Legend: NHCbz is

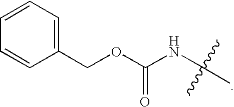

Compositions

In a second aspect, the invention provides compositions comprising a compound according to the invention or an N-oxide, hydrate, solvate, pharmaceutically acceptable salt, complex or prodrug thereof, or a racemic or scalemic mixture, diastereomer, enantiomer or tautomer thereof, and a pharmaceutically acceptable carrier, excipient, or diluent. Compounds of the invention may be formulated by any method well known in the art and may be prepared for administration by any route, including, without limitation, parenteral, oral, sublingual, transdermal, topical, intranasal, intratracheal, intravenous or intrarectal. In certain preferred embodiments, compounds of the invention are administered intravenously in a hospital setting. In certain other preferred embodiments, administration may preferably be by the oral route. The compositions may be in the form of liquid solutions or suspensions; for oral administration, formulations may be in the form of tablets or capsules; and for intranasal formulations, in the form of powders, nasal dropsa or aerosols. The compositions of the invention may be administered systemically or locally.

The characteristics of the carrier will depend on the route of administration. As used herein, the term “pharmaceutically acceptable” means a non-toxic material that is compatible with a biological system such as a cell, cell culture, tissue, or organism, and that does not interfere with the effectiveness of the biological activity of the active ingredient(s). Thus, compositions according to the invention may contain, in addition to the inhibitor, diluents, fillers, salts, buffers, stabilizers, solubilizers, and other materials well known in the art. The preparation of pharmaceutically acceptable formulations is described in, e.g., Remington's Pharmaceutical Sciences, 18th Edition, ed. A. Gennaro, Mack Publishing Co., Easton, Pa., 1990.

In a preferred embodiment of the second aspect, the composition comprises a compound, N-oxide, hydrate, solvate, pharmaceutically acceptable salt, complex or prodrug of a compound according to the present invention as described herein present in at least about 30% enantiomeric or diastereomeric excess. In certain desirable embodiments of the invention, the compound, N-oxide, hydrates, solvate, pharmaceutically acceptable salt, complex or prodrug is present in at least about 50%, at least about 80%, or even at least about 90% enantiomeric or diastereomeric excess. In certain other desirable embodiments of the invention, the compound, N-oxide, hydrate, solvate, pharmaceutically acceptable salt, complex or prodrug is present in at least about 95%, more preferably at least about 98% and even more preferably at least about 99% enantiomeric or diastereomeric excess. In other embodiments of the invention, a compound, N-oxide, hydrate, solvate, pharmaceutically acceptable salt, complex or prodrug is present as a substantially racemic mixture. In a preferred embodiment, the composition further comprises an additional therapeutic or inhibitory agent.

As used herein, the term “pharmaceutically acceptable salts” is intended to mean salts that retain the desired biological activity of the above-identified compounds and exhibit minimal or no undesired toxicological effects. Examples of such salts include, but are not limited to acid addition salts formed with inorganic acids (for Example, hydrochloric acid, hydrobromic acid, sulfuric acid, phosphoric acid, nitric acid, and the like), and salts formed with organic acids such as acetic acid, oxalic acid, tartaric acid, succinic acid, malic acid, ascorbic acid, benzoic acid, tannic acid, pamoic acid, alginic acid, polyglutamic acid, naphthalenesulfonic acid, naphthalenedisulfonic acid, and polygalacturonic acid. The compounds can also be administered as pharmaceutically acceptable quaternary salts known by those skilled in the art, which specifically include the quaternary ammonium salt of the formula —NR+Z-, wherein R is hydrogen, alkyl, or benzyl, and Z is a counterion, including chloride, bromide, iodide, —O-alkyl, toluenesulfonate, methylsulfonate, sulfonate, phosphate, or carboxylate (such as benzoate, succinate, acetate, glycolate, maleate, malate, citrate, tartrate, ascorbate, benzoate, cinnamoate, mandeloate, benzyloate, and diphenylacetate). As used herein, the term “salt” is also meant to encompass complexes, such as with an alkaline metal or an alkaline earth metal.

The active compound is included in the pharmaceutically acceptable carrier or diluent in an amount sufficient to deliver an inhibition effective amount without causing serious toxic effects. A preferred dose of the active compound for all of the above-mentioned conditions is in the range from about 0.01 to 300 mg/kg, preferably 0.1 to 100 mg/kg per day, more generally 0.5 to about 25 mg per kilogram body weight of the recipient per day. A typical topical dosage will range from 0.01-3% wt/wt in a suitable carrier. The effective dosage range of the pharmaceutically acceptable derivatives can be calculated based on the weight of the parent compound to be delivered. If the derivative exhibits activity in itself, the effective dosage can be estimated as above using the weight of the derivative, or by other means known to those skilled in the art.

In certain preferred embodiments of the second aspect of the invention, the composition further comprises an antisense oligonucleotide that inhibits the expression of a histone deacetylase gene. The combined use of a nucleic acid level inhibitor (e.g., antisense oligonucleotide) and a protein level inhibitor (i.e., inhibitor of histone deacetylase enzyme activity) results in an improved inhibitory effect, thereby reducing the amounts of the inhibitors required to obtain a given inhibitory effect as compared to the amounts necessary when either is used individually. The antisense oligonucleotides according to this aspect of the invention are complementary to regions of RNA or double-stranded DNA that encode HDAC-1, HDAC-2, HDAC-3, HDAC-4, HDAC-5, HDAC-6, HDAC-7, HDAC-8, HDAC-9, HDAC-10, HDAC-11 (see e.g., GenBank Accession Number U50079 for HDAC-1, GenBank Accession Number U31814 for HDAC-2, and GenBank Accession Number U75697 for HDAC-3).

Additional HDAC inhibitory agents may also be present in the compositions of this invention, where the combination causes no unacceptable adverse effects.

Examples of additional HDAC inhibitors include, but are not limited to, SAHA, MS-275, MGCD0103, PXD101, NVP-LAQ824, LBH589, cyclic tetrapeptides (desipeptide, or Romidepsin®) and those described in WO 03/024448, WO 2004/069823, US 2006/0058298, US 2005/0288282, WO 00/071703, WO 01/38322, WO 01/70675, WO 03/006652, WO 2004/035525, WO 2005/030705, WO 2005/092899.

Inhibition of Histone Deacetylase

In a third aspect, the invention provides a method of inhibiting histone deacetylase, the method comprising contacting the histone deacetylase with an inhibition effective amount of a compound according to the present invention, or with an inhibition effective amount of a composition according to the present invention. Inhibition of histone deacetylase activity can be in a cell or a multicellular organism. If in a cell, the method according to this aspect comprises contacting the cell with an inhibition effective amount of a compound according to the present invention, or with an inhibition effective amount of a composition according to the present invention. If in a multicellular organism, the method according to this aspect of the invention comprises administering to the organism an inhibition effective amount of a compound according to the present invention, or an inhibition effective amount of a composition according to the present invention. Preferably the organism is a mammal, more preferably a human. In a preferred embodiment, the method further comprises contacting the histone deacetylase, or the cell, with an effective amount of an additional HDAC inhibitory agent, or if in a multicellular organism, concurrently or sequentially administering an inhibition effective amount of an additional HDAC inhibitory agent.

In another preferred embodiment, the method is a method of treating a disease responsive to an inhibitor of HDAC and comprises administering to an individual in need thereof an effective amount of a compound according to the present invention. In certain preferred embodiments, the method of treatment further comprises administering an effective amount of an additional therapeutic agent, wherein the additional therapeutic agent is a therapeutic agent appropriate for treating the disease.

Because compounds of the invention inhibit histone deacetylase, they are useful research tools for in vitro study histone deacetylases and their role in biological processes.

Measurement of the enzymatic activity of a histone deacetylase can be achieved using known methodologies. For Example, Yoshida et al., J. Biol. Chem., 265: 17174-17179 (1990), describes the assessment of histone deacetylase enzymatic activity by the detection of acetylated histones in trichostatin A treated cells. Taunton et al., Science, 272: 408-411 (1996), similarly describes methods to measure histone deacetylase enzymatic activity using endogenous and recombinant HDAC-1.

In some preferred embodiments, the histone deacetylase inhibitor interacts with and reduces the activity of all histone deacetylases in a cell. In some other preferred embodiments according to this aspect of the invention, the histone deacetylase inhibitor interacts with and reduces the activity of fewer than all histone deacetylases in the cell. In certain preferred embodiments, the inhibitor interacts with and reduces the activity of one histone deacetylase (e.g., HDAC-1), but does not interact with or reduce the activities of other histone deacetylases (e.g., HDAC-2, HDAC-3, HDAC-4, HDAC-5, HDAC-6, HDAC-7, HDAC-8, HDAC-9, HDAC-10, HDAC-11).

The term “inhibition effective amount” is meant to denote a dosage sufficient to cause inhibition of histone deacetylase activity in a cell, which cell can be in a multicellular organism. The multicellular organism can be a plant or an animal, preferably a mammal, more preferably a human If in a multicellular organism, the method according to this aspect of the invention comprises administering to the organism a compound or composition according to the present invention. Administration may be by any route, including, without limitation, parenteral, oral, sublingual, transdermal, topical, intranasal, intratracheal, intravenous or intrarectal. In certain particularly preferred embodiments, compounds of the invention are administered intravenously in a hospital setting. In certain other preferred embodiments, administration may preferably be by the oral route.

In certain preferred embodiments of the third aspect of the invention, the method further comprises contacting a histone deacetylase enzyme or a cell expressing histone deacetylase activity with an antisense oligonucleotide that inhibits the expression of a histone deacetylase gene. The combined use of a nucleic acid level inhibitor (e.g., antisense oligonucleotide) and a protein level inhibitor (i.e., inhibitor of histone deacetylase enzyme activity) results in an improved inhibitory effect, thereby reducing the amounts of the inhibitors required to obtain a given inhibitory effect as compared to the amounts necessary when either is used individually. The antisense oligonucleotides according to this aspect of the invention are complementary to regions of RNA or double-stranded DNA that encode HDAC-1, HDAC-2, HDAC-3, HDAC-4, HDAC-5, HDAC-6, HDAC-7, HDAC-8, HDAC-9, HDAC-10 or HDAC-11 (see e.g., GenBank Accession Number U50079 for HDAC-1, GenBank Accession Number U31814 for HDAC-2, and GenBank Accession Number U75697 for HDAC-3).

For purposes of the invention, the term “oligonucleotide” includes polymers of two or more deoxyribonucleosides, ribonucleosides, or 2′-substituted ribonucleoside residues, or any combination thereof. Preferably, such oligonucleotides have from about 6 to about 100 nucleoside residues, more preferably from about 8 to about 50 nucleoside residues, and most preferably from about 12 to about 30 nucleoside residues. The nucleoside residues may be coupled to each other by any of the numerous known internucleoside linkages. Such internucleoside linkages include without limitation phosphorothioate, phosphorodithioate, alkylphosphonate, alkylphosphonothioate, phosphotriester, phosphoramidate, siloxane, carbonate, carboxymethylester, acetamidate, carbamate, thioether, bridged phosphoramidate, bridged methylene phosphonate, bridged phosphorothioate and sulfone internucleoside linkages. In certain preferred embodiments, these internucleoside linkages may be phosphodiester, phosphotriester, phosphorothioate, or phosphoramidate linkages, or combinations thereof. The term oligonucleotide also encompasses such polymers having chemically modified bases or sugars and/or having additional substituents, including without limitation lipophilic groups, intercalating agents, diamines and adamantane.

For purposes of the invention the term “2′-substituted ribonucleoside” includes ribonucleosides in which the hydroxyl group at the 2′ position of the pentose moiety is substituted to produce a 2′-O-substituted ribonucleoside. Preferably, such substitution is with a lower alkyl group containing 1-6 saturated or unsaturated carbon atoms, or with an aryl or allyl group having 2-6 carbon atoms, wherein such alkyl, aryl or allyl group may be unsubstituted or may be substituted, e.g., with halo, hydroxy, trifluoromethyl, cyano, nitro, acyl, acyloxy, alkoxy, carboxyl, carbalkoxyl, or amino groups. The term “2′-substituted ribonucleoside” also includes ribonucleosides in which the 2′-hydroxyl group is replaced with an amino group or with a halo group, preferably fluoro.

Particularly preferred antisense oligonucleotides utilized in this aspect of the invention include chimeric oligonucleotides and hybrid oligonucleotides.

For purposes of the invention, a “chimeric oligonucleotide” refers to an oligonucleotide having more than one type of internucleoside linkage. One preferred example of such a chimeric oligonucleotide is a chimeric oligonucleotide comprising a phosphorothioate, phosphodiester or phosphorodithioate region, preferably comprising from about 2 to about 12 nucleotides, and an alkylphosphonate or alkylphosphonothioate region (see e.g., Pederson et al. U.S. Pat. Nos. 5,635,377 and 5,366,878). Preferably, such chimeric oligonucleotides contain at least three consecutive internucleoside linkages selected from phosphodiester and phosphorothioate linkages, or combinations thereof.

For purposes of the invention, a “hybrid oligonucleotide” refers to an oligonucleotide having more than one type of nucleoside. One preferred example of such a hybrid oligonucleotide comprises a ribonucleotide or 2′-substituted ribonucleotide region, preferably comprising from about 2 to about 12 2′-substituted nucleotides, and a deoxyribonucleotide region. Preferably, such a hybrid oligonucleotide contains at least three consecutive deoxyribonucleosides and also contains ribonucleosides, 2′-substituted ribonucleosides, preferably 2′-O-substituted ribonucleosides, or combinations thereof (see e.g., Metelev and Agrawal, U.S. Pat. No. 5,652,355).

The exact nucleotide sequence and chemical structure of an antisense oligonucleotide utilized in the invention can be varied, so long as the oligonucleotide retains its ability to inhibit expression of the gene of interest. This is readily determined by testing whether the particular antisense oligonucleotide is active. Useful assays for this purpose include quantitating the mRNA encoding a product of the gene, a Western blotting analysis assay for the product of the gene, an activity assay for an enzymatically active gene product, or a soft agar growth assay, or a reporter gene construct assay, or an in vivo tumor growth assay, all of which are described in detail in this specification or in Ramchandani et al. (1997) Proc. Natl. Acad. Sci. USA 94: 684-689.

Antisense oligonucleotides utilized in the invention may conveniently be synthesized on a suitable solid support using well known chemical approaches, including H-phosphonate chemistry, phosphoramidite chemistry, or a combination of H-phosphonate chemistry and phosphoramidite chemistry (i.e., H-phosphonate chemistry for some cycles and phosphoramidite chemistry for other cycles). Suitable solid supports include any of the standard solid supports used for solid phase oligonucleotide synthesis, such as controlled-pore glass (CPG) (see, e.g., Pon, R. T. (1993) Methods in Molec. Biol. 20: 465-496).

In certain preferred embodiments of the invention, the antisense oligonucleotide and the HDAC inhibitor of the present invention are administered separately to a mammal, preferably a human. For example, the antisense oligonucleotide may be administered to the mammal prior to administration to the mammal of the HDAC inhibitor of the present invention. The mammal may receive one or more dosages of antisense oligonucleotide prior to receiving one or more dosages of the HDAC inhibitor of the present invention.

In another embodiment, the HDAC inhibitor of the present invention may be administered to the mammal prior to administration of the antisense oligonucleotide. The mammal may receive one or more dosages of the HDAC inhibitor of the present invention prior to receiving one or more dosages of antisense oligonucleotide.

In certain preferred embodiments of the present invention, the HDAC inhibitor of the present invention may be administered together with another HDAC inhibitor known in the art or which will be discovered. Administration of such HDAC inhibitors may be done sequentially or concurrently. In certain preferred embodiments of the present invention the compositions comprise an HDAC inhibitor of the present invention and/or an antisense oligonucleotide and/or another HDAC inhibitor known in the art or which will be discovered. The active ingredients of such compositions preferably act synergistically to produce a therapeutic effect.

Examples of other HDAC inhibitors include, but are not limited to, TSA, depudecin, trapoxin, CI-994, sodium butyrate, SAHA, MS-275, MGCD0103, PXD101, NVP-LAQ824, LBH589, cyclic tetrapeptides (desipeptide, or Romidepsin®) and those described in WO 03/024448, WO 2004/069823, US 2006/0058298, US 2005/0288282, WO 00/071703, WO 01/38322, WO 01/70675, WO 03/006652, WO 2004/035525, WO 2005/030705, WO 2005/092899, U.S. Pat. No. 6,541,661.

The following Examples are intended to further illustrate certain preferred embodiments of the invention, and are not intended to limit the scope of the invention.

### ASSAY EXAMPLES

### Assay Example 1

### Inhibition of Histone Deacetylase Enzymatic Activity

The following protocol is used to assay the compounds of the invention. In the assay, the buffer used is 25 mM HEPES, pH 8.0, 137 mM NaCl, 2.7 mM KCl, 1 mM MgCl₂ and the subtrate is Boc-Lys(Ac)-AMC in a 50 mM stock solution in DMSO. The enzyme stock solution is 4.08 μg/mL in buffer.

The compounds are pre-incubated (2 μl in DMSO diluted to 13 μl in buffer for transfer to assay plate) with enzyme (20 μl of 4.08 μg/ml) for 10 minutes at room temperature (35 μl pre-incubation volume). The mixture is pre-incubated for 5 minutes at room temperature. The reaction is started by bringing the temperature to 37° C. and adding 16 μl substrate. Total reaction volume is 50 μl. The reaction is stopped after 20 minutes by addition of 50 μl developer, prepared as directed by Biomol (Fluor-de-Lys developer, Cat. #KI-105). A plate is incubated in the dark for 10 minutes at room temperature before reading (λ_{Ex}=360 nm, λ_{Em}=470 nm, Cutoff filter at 435 nm).

### Assay Example 2

Alternatively, HDAC inhibitory activity was determined using the following assay.

A 30 mM stock of Boc-Lys(trifluoroacetyl)-AMC substrate is prepared in DMSO. 2 μL of test compound in DMSO is diluted to 50 μL in buffer (25 mM HEPES, pH 8.0, 137 mM NaCl, 2.7 mM KCl, 1 mM MgCl₂, 0.1% BSA) and pre-incubated with HDAC enzyme (30 μL of a final enzyme concentration of 0.1-0.2 nM) for 10 minutes at room temperature. Reaction is started by adding 18 μL Boc-Lys(trifluoroacetyl)-AMC substrate and incubating at 37° C. for 20-30 minutes. The reaction is stopped by adding 50 μL trypsin (1 mg/mL) and a known HDAC inhibitor (such as LAQ-824). The plate is then incubated in the dark for 20 minutes at room temperature and read with Ex=360 nm, Em=470 nm, cutoff filter at 435 nm.

All compounds exemplified in the application show inhibitory activity against one or more of HDAC-1, HDAC-2, HDAC-3, HDAC-4, HDAC-5, HDAC-6, HDAC-7, HDAC-8, HDAC-9,HDAC-10 and HDAC-11. The IC₅₀ values of selected compounds exemplified in the application are shown in Table 3. In the table, A≦0.2 μM; 0.2 μM<B≦0.5 μM; 0.5 M<C≦1 μM; and 1 μM<D≦6 μM.

TABLE 3

| IC₅₀ | Cpd Number | IC₅₀ | Cpd Number | IC₅₀ | Cpd Number |
|---|---|---|---|---|---|
| A | 4 | B | 289 | A | 313 |
| B | 43 | A | 78 | A | 314 |
| A | 40 | B | 207 | A | 230a |
| B | 51 | B | 201 | A | 242 |
| A | 55 | B | 203 | A | 335 |
| A | 34 | B | 209 | A | 327 |
| A | 60 | A | 79 | B | 361 |
| B | 62 | A | 211 | C | 364 |
| A | 270 | A | 308 | B | 318 |
| A | 274 | B | 309 | B | 348 |
| A | 319 | B | 240 | | |
| B | 252 | A | 310 | | |

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth, and as follows in the scope of the appended claims.

What is claimed is:

1. A compound of the formula (I), and racemic and scalemic mixtures, diastereomers and enantiomers thereof: 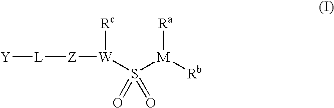
or an N-oxide, hydrate, solvate, pharmaceutically acceptable salt, prodrug or complex thereof, wherein
M is nitrogen or oxygen; wherein when M is oxygen, R^{b} is absent and W is nitrogen;
W is nitrogen or oxygen; wherein when W is oxygen, R^{c} is absent and M is nitrogen;
R^{a} is independently selected from the group consisting of —H, —C₁-C₆alkyl, a protecting group, —C₁-C₆alkyl-aryl, aryl, —C₁-C₆alkyl-heteroaryl, heteroaryl, —C₁-C₆alkyl-cycloalkyl, cycloalkyl, —C₁-C₆alkyl-heterocyclyl, heterocyclyl, —C(O)—O—C₁-C₆alkyl, —C(O)—O—C₁-C₆alkyl-heterocyclyl, —C(O)—O—C₁-C₆alkyl-alkenyl, —C(O)—O—C₁-C₆alkyl-aryl, —CO—CF₃ and 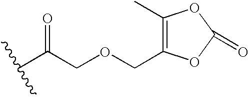
each of which is optionally substituted; and
when M is nitrogen, R^{a} is further selected from —C(O)—H;
R^{b} and R^{c}, when present, are independently selected from the group consisting of —H, —OH, —CN, —O-alkyl, —C₁-C₆alkyl, —C(O)-alkyl, —NH₂, —NH-alkyl, —C(O)—H, a protecting group, —C₁-C₆alkyl-aryl, aryl, —C₁-C₆alkyl-heteroaryl, -heteroaryl, —C₁-C₆alkyl-cycloalkyl, cycloalkyl, —C₁-C₆alkyl-heterocyclyl, heterocyclyl, —C(O)—C₀-C₃alkyl-aryl, —C(O)—C₀-C₃alkyl-heteroaryl, —C(O)—C₀-C₃alkyl-cycloalkyl, —C(O)—C₀-C₃alkyl-heterocycyl), —C(O)—O—C₁-C₆alkyl, —C(O)—O—C₁-C₆alkyl-heterocyclyl, —C(O)—O—C₁-C₆alkyl-alkenyl, —C(O)—O—C₁-C₆alkyl-aryl, —CO—CF₃ and 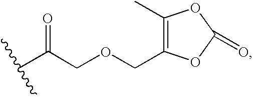
each of which is optionally substituted;
wherein, when M is nitrogen, R^{a} and R^{b} together with the nitrogen atom to which they are attached optionally form a 3 to 9-membered heterocyclyl, heteroaryl, heterocyclyl-aryl or heterocyclyl-heteroaryl, each of which is optionally substituted; and
wherein, when R^{a} is —C₁-C₆alkyl, —C₁-C₆alkyl-aryl, —C₁-C₆alkyl-heteroaryl, —C₁-C₆alkyl-heterocyclyl, or —C₁-C₆alkyl-cycloalkyl and R^{c} is —C₁-C₆alkyl, then R^{a} and R^{c} are optionally connected with a carbon atom to form a 5 to 10-membered heterocyclyl, heterocyclyl-aryl, heterocyclyl-heteroaryl, heterocyclyl-heterocyclyl or heterocyclyl-cycloalkyl, each of which is optionally substituted;
Z is selected from the group consisting of a covalent bond, —C₃-C₈alkyl-, —C₀-C₃alkyl-C₁-C₈heteroalkyl-C₀-C₃alkyl-, —C₀-C₃alkyl-C₂-C₅alkenyl-C₀-C₃alkyl-, —C₀-C₃alkyl-C₂-C₅alkynyl-C₀-C₃alkyl-, —C₀-C₆alkyl-aryl-C₀-C₆alkyl-, —C₀-C₆alkyl-aryl-C₂-C₆heteroalkyl-, —C₀-C₆alkyl-cycloalkyl-C₀-C₆alkyl-, —C₀-C₆alkyl-cycloalkyl-C₂-C₆heteroalkyl-, —C₄-C₆heterocyclyl-aryl-C₀-C₆alkyl-, —C₄-C₆heterocyclyl-aryl-C₀-C₆heteroalkyl-, —C₀-C₆alkyl-C₄-C₆-heterocyclyl-C₀-C₆alkyl-, —C₀-C₆alkyl-C₄-C₆-heterocyclyl-C₀-C₆heteroalkyl-, —C₀-C₆-alkyl-heteroaryl-C₀-C₆alkyl-, —C₀-C₆-alkyl-heteroaryl-C₀-C₆heteroalkyl-, —C₄-C₆heterocyclyl-heteroaryl-C₀-C₆alkyl-, —C₄-C₆heterocyclyl-heteroaryl-C₀-C₆heteroalkyl-, —C₀-C₆alkyl-aryl-C₃-C₆alkynyl-, —C₀-C₆alkyl-heteroaryl-C₂-C₆alkynyl-, —C₀-C₆alkyl-cycloalkyl-C₃-C₆alkynyl-, —C₀-C₆alkyl-heterocyclyl-C₃-C₆alkynyl-, —C₀-C₆alkyl-aryl-C₂-C₆alkynyl-C₂-C₆alkenyl-, —C₀-C₆alkyl-aryl-C₂-C₆alkenyl-, —C₀-C₆alkyl-heteroaryl-C₂-C₆alkenyl-, —C₀-C₆alkyl-cycloalkyl-C₂-C₆alkenyl-, —C₀-C₆alkyl-heterocyclyl-C₂-C₆alkenyl-, —C₀-C₆alkyl-aryl-aryl-C₀-C₆alkyl-, —C₀-C₆alkyl-aryl-heteroaryl-C₀-C₆alkyl-, —C₀-C₆alkyl-heteroaryl-aryl-C₀-C₆-alkyl-, —CO—C₆alkyl-heteroaryl-heteroaryl-C₀-C₆-alkyl-, —C₀-C₃alkyl-heteroaryl-C₀-C₃alkyl-C₂-C₈ alkenyl-C₀-C₃-alkyl-, —C₀-C₃alkyl-aryl-C₀-C₃alkyl-C₂-C₈ alkenyl-C₀-C₃-alkyl-, —C₀-C₃alkyl-heterocyclyl-C₀-C₃alkyl-C₂-C₈ alkenyl-C₀-C₃-alkyl-, —C₀-C₃alkyl-cycloalkyl-C₀-C₃alkyl-C₂-C₈ alkenyl-C₀-C₃-alkyl-, —C₀-C₃alkyl-heteroaryl-C₀-C₃alkyl-C₂-C₈ alkynyl-C₀-C₃-alkyl-, —C₀-C₃alkyl-aryl-C₀-C₃alkyl-C₂-C₈ alkynyl-C₀-C₃-alkyl-, —C₀-C₃alkyl-heterocyclyl-C₀-C₃alkyl-C₂-C₈ alkynyl-C₀-C₃-alkyl- and —CO—C₃alkyl-cycloalkyl-C₀-C₃alkyl-C₂-C₈ alkynyl-C₀-C₃-alkyl-, wherein each alkyl, alkenyl, alkynyl, heteroalkyl, aryl, heteroaryl, heterocyclyl, and cycloalkyl moiety is optionally substituted; and
when W is N, Z is further selected from the group consisting of —C₁-C₈alkyl-C(O)—, —C₁-C₈ alkyl-S(O)₂—, -aryl-C₀-C₈ alkyl-S(O)₂—, -heteroaryl-C₀-C₈ alkyl-S(O)₂—, -cycloalkyl-C₀-C₈ alkyl-S(O)₂—, -heterocycloalkyl-C₀-C₈ alkyl-S(O)₂—, —C₁-C₈ alkyl-CH═, —C₁-C₈ alkyl-C(CH₃)═, —C₀-C₆alkyl-CH═CH—C(O), —C₀-C₄alkyl-C(O)—, —C₁-C₈ alkyl-(NH₂)C═, —C₀-C₃alkyl-C₁-C₈heteroalkyl-C₀-C₃alkyl-C(O)—, —C₀-C₃alkyl-C₂-C₈alkenyl-C₀-C₃alkyl-C(O)—, —C₀-C₃alkyl-C₂-C₈alkynyl-C₀-C₃alkyl-C(O)—, —C₀-C₆alkyl-aryl-C₀-C₆alkyl-C(O)—, —C₀-C₆alkyl-aryl-C₂-C₆heteroalkyl-C(O)—, —C₀-C₆alkyl-cycloalkyl-C₀-C₆alkyl-C(O)—, —C₀-C₆alkyl-cycloalkyl-C₂-C₆heteroalkyl-C(O)—, —C₄-C₆heterocyclyl-aryl-C₀-C₆alkyl-C(O)—, —C₄-C₆heterocyclyl-aryl-C₀-C₆heteroalkyl-C(O)—, —C₀-C₆alkyl-C₄-C₆-heterocyclyl-C₀-C₆alkyl-C(O)—, —C₀-C₆alkyl-C₄-C₆-heterocyclyl-C₀-C₆heteroalkyl-C(O)—, —C₀-C₆-alkyl-heteroaryl-C₀-C₆alkyl-C(O)—, —C₀-C₆-alkyl-heteroaryl-C₀-C₆heteroalkyl-C(O)—, —C₄-C₆heterocyclyl-heteroaryl-C₀-C₆alkyl-C(O)—, —C₄-C₆heterocyclyl-heteroaryl-C₀-C₆heteroalkyl-C(O)—, —C₀-C₆alkyl-aryl-C₃-C₆alkynyl-C(O)—, —C₀-C₆alkyl-heteroaryl-C₂-C₆alkynyl-C(O)—, —C₀-C₆alkyl-cycloalkyl-C₃-C₆alkynyl-C(O)—, —C₀-C₆alkyl-heterocyclyl-C₃-C₆alkynyl-C(O)—, —C₀-C₆alkyl-aryl-C₂-C₆alkynyl-C₂-C₆alkenyl-C(O)—, —C₀-C₆alkyl-aryl-C₂-C₆alkenyl-C(O)—, —C₀-C₆alkyl-heteroaryl-C₂-C₆alkenyl-C(O)—, —C₀-C₆alkyl-cycloalkyl-C₂-C₆alkenyl-C(O)—, —C₀-C₆alkyl-heterocyclyl-C₂-C₆alkenyl-C(O)—, —C₀-C₆alkyl-aryl-aryl-C₀-C₆alkyl-C(O)—, —C₀-C₆alkyl-aryl-heteroaryl-C₀-C₆alkyl-C(O)—, —C₀-C₆alkyl-heteroaryl-aryl-C₀-C₆-alkyl-C(O)— and —C₀-C₆alkyl-heteroaryl-heteroaryl-C₀-C₆-alkyl-C(O)—; or
Z-W is selected from the group consisting of —C₁-C₈ alkyl-(NH₂)C═N—, —C₁₋₈alkyl-C═N— and —C₁₋₈alkyl-C(CH₃)═N—, when R^{c} is absent;
L is selected from the group consisting of a covalent bond, —C₁-C₆alkyl-, —C₀-C₆alkyl-(CR³═CR³)₁₋₂—CO—C₆alkyl-, —C₀-C₆alkyl-(C≡C)₁₋₂—C₀-C₆alkyl-, —C₀-C₆alkyl-aryl-C₀-C₆alkyl-, —C₀-C₆alkyl-heteroaryl-C₀-C₆alkyl-, —C₀-C₃alkyl-heterocyclyl-C₀-C₃alkyl-, —C₀-C₃alkyl-cycloalkyl-C₀-C₃alkyl-, —C₀-C₆alkyl-aryl-(CR³═CR³)₁₋₂—C₀-C₆alkyl-, —C₀-C₆ alkyl-aryl-(C≡C)₁₋₂—C₀-C₆alkyl-, —C₀-C₆alky-heteroaryl-(CR³═CR³)₁₋₂—C₀-C₆alkyl-, —C₀-C₆ alkyl-heteroaryl-(C≡C)₁₋₂—C₀-C₆alkyl-, —C₀-C₆alkyl-N(R³)—C(O)—C₀-C₃alkyl-, —C₀-C₆alkyl-N(R³)—C(S)—C₀-C₃alkyl-, —C₀-C₆alkyl-N(R³)—C(O)-alkenyl-C₀-C₃alkyl-, —C₀-C₆alkyl-N(R³)—C(S)-alkenyl-C₀-C₃alkyl-, —C₀-C₆alkyl-N(R³)—C(O)-alkynyl-C₀-C₃alkyl-, —C₀-C₆alkyl-N(R³)—C(S)-alkynyl-C₀-C₃alkyl-, —C₀-C₆alkyl-N(R³)—C(O)—C₁-C₃alkyl-N(R³)—C(O)—, —CO—C₆alkyl-N(R³)—C(S)—C₁-C₃alkyl-N(R³)—C(O)—, —C₀-C₆alkyl-N(R³)—C(O)—C₁-C₃alkyl-N(R³)—C(O)—C₀-C₃alkyl-, —C₀-C₆alkyl-N(R³)—C(S)—C₁-C₃alkyl-N(R³)—C(O)—C₀-C₃alkyl-, —C₀-C₆alkyl-N(R³)—C(O)—C₁-C₃alkyl-N(R³)—C(S)—C₀-C₃alkyl-, —C₀-C₆alkyl-N(R³)—C(S)—C₁-C₃alkyl-N(R³)—C(S)—C₀-C₃alkyl-, —C₀-C₆alkyl-C(O)—N(R³)—C₀-C₃alkyl-, —C₀-C₆alkyl-C(S)—N(R³)—C₀-C₃alkyl-, —C₀-C₆alkyl-C(O)—N(R³)—C₂-C₄alkyl-O—C₀-C₃alkyl-, —C₀-C₆alkyl-C(S)—N(R³)—C₂-C₄alkyl-O—C₀-C₃alkyl-, —C₀-C₆alkyl-C(O)—N(R³)—C₂-C₄alkyl-N(R³)—C₀-C₃alkyl-, —C₀-C₆alkyl-C(S)—N(R³)—C₂-C₄alkyl-N(R³)—C₀-C₃alkyl-, —C₀-C₆alkyl-C(O)—C₀-C₃alkyl-, —C₀-C₆alkyl-C(NOH)—C₀-C₃alkyl-, —C₀-C₆alkyl-C(O)-alkenyl-C₀-C₃alkyl-, —C₀-C₆alkyl-C(NOH)-alkenyl-C₀-C₃alkyl-, —C₀-C₆alkyl-S(O)₂—N(R³)—C₀-C₃alkyl-, —C₀-C₆alkyl-N(R³)—S(O)₂—C₀-C₃alkyl-, —C₀-C₃alkyl-N(R³)— S(O)₂—N(R³)—C₀-C₃alkyl-, —C₀-C₆alkyl-C(O)—N(R³)—S(O)₂—C₀-C₃alkyl-, —C₀-C₆alkyl-N(R³)—C₀-C₃alkyl-, —C₀-C₆alkyl-N(R³)—C₁-C₃alkyl-alkenyl-, —C₀-C₆alkyl-N(R³)—C₁-C₃alkyl-alkynyl-, —C₀-C₆alkyl-N(R⁷)—C₀-C₃alkyl-, —C₀-C₆alkyl-N(R⁷)—C₁-C₃alkyl-alkenyl-, —C₀-C₆alkyl-N(R⁷)—C₁-C₃alkyl-alkynyl-, —C₀-C₆alkyl-N(R³)—C₂-C₄alkyl-N(R³)—C(O)-alkyl-, —C₀-C₆alkyl-N(R³)—C₂-C₄alkyl-N(R³)—C(S)-alkyl-, —C₀-C₆alkyl-O—C₂-C₄alkyl-N(R³)C(O)—C₀-C₃alkyl-, —C₀-C₆alkyl-S—C₂-C₄alkyl-N(R³)C(O)—C₀-C₃alkyl-, —C₀-C₆alkyl-O—C₂-C₄alkyl-N(R³)C(S)—C₀-C₃alkyl-, —C₀-C₆alkyl-S—C₂-C₄alkyl-N(R³)C(S)—C₀-C₃alkyl-, —C₀-C₆alkyl-N(R³)—C₂-C₃heteroalkyl-, —C₀-C₆alkyl-N(R⁷)—C₂-C₃heteroalkyl-, —C₀-C₆alkyl-C(O)—N(R³)—C₂-C₃heteroalkyl-, —C₀-C₆alkyl-C(S)—N(R³)—C₂-C₃heteroalkyl-, —C₀-C₆alkyl-C(O)—N(R⁷)—C₂-C₃heteroalkyl-, —C₀-C₆alkyl-C(S)—N(R⁷)—C₂-C₃heteroalkyl-, —C₀-C₆alkyl-N(R³)—C(O)—C₀-C₃heteroalkyl-, —C₀-C₆alkyl-N(R³)—C(S)—C₀-C₃heteroalkyl-, —C₀-C₆alkyl-N(R⁷)—C(O)—C₀-C₃heteroalkyl-, —C₀-C₆alkyl-N(R⁷)—C(S)—C₀-C₃heteroalkyl-, —C₀-C₆alkyl-S—C₀-C₃alkyl-, —C₀-C₆alkyl-O—C₀-C₃alkyl-, —C₀-C₆alkyl-S(O)—C₀-C₃alkyl-, —C₀-C₆alkyl-S(O)₂—C₀-C₃alkyl-, —C₀-C₆alkyl-N(R³)—C(O)—N(R³)—C₀-C₃alkyl-, —C₀-C₆alkyl-N(R³)—C(S)—N(R³)—C₀-C₃alkyl-, —C₀-C₆alkyl-O—C₀-C₃alkyl-heterocyclyl-, —C₀-C₆alkyl-O—C₀-C₃alkyl-cycloalkyl-, —C₀-C₆alkyl-S(O)₀₋₂—C₀-C₃alkyl-heterocycly-, —C₀-C₆alkyl-S(O)₀₋₂—C₀-C₃alkyl-cycloalkyl-, —C₀-C₆alkyl-N(R³)—C₀-C₃alkyl-heterocycly-, —C₀-C₆alkyl-N(R³)—C₀-C₃alkyl-cycloalkyl-, -heterocyclyl-C₀-C₆alkyl-O—C₀-C₃alkyl-, -cycloalkyl-C₀-C₆alkyl-O—C₀-C₃alkyl-, -heterocyclyl-C₀-C₆alkyl-S(O)₀₋₂—C₀-C₃alkyl-, -cycloalkyl-C₀-C₆alkyl-S(O)₀₋₂—C₀-C₃alkyl-, -heterocyclyl-C₀-C₆alkyl-N(R³)—C₀-C₃alkyl-, -cycloalkyl-C₀-C₆alkyl-N(R³)—C₀-C₃alkyl-, —C₀-C₆alkyl-N(R³)—C(O)—O—C₀-C₃alkyl-, —C₀-C₆alkyl-N(R³)—C(S)—O—C₀-C₃alkyl-, —C₀-C₆alkyl-O—C(O)—N(R³)—C₀-C₃alkyl-, —C₀-C₆alkyl-O—C(S)—N(R³)—C₀-C₃alkyl-, —C₀-C₃alkyl-N(R³)—C(O)—O-heterocyclyl-C₀-C₃alkyl-, —C₀-C₃alkyl-N(R³)—C(S)—O-heterocyclyl-C₀-C₃alkyl-, —C₀-C₃alkyl-N(R³)—C(O)—O-cycloalkyl-C₀-C₃alkyl-, —C₀-C₃alkyl-N(R³)—C(S)—O-cycloalkyl-C₀-C₃alkyl-, —C₀-C₆alkyl-S(O)₂-heterocyclyl-C₀-C₃alkyl-, —C₀-C₆alkyl-S(O)₂-cycloalkyl-C₀-C₃alkyl-, —C₀-C₆ alkyl-N(R³)—S(O)₂-heterocyclyl-C₀-C₃alkyl-, —C₀-C₆ alkyl-N(R³)—S(O)₂-cycloalkyl-C₀-C₃alkyl-, —C₀-C₃alkyl-heterocyclyl-C₀-C₃alkyl-O—C₀-C₃alkyl-, —C₀-C₃alkyl-cycloalkyl-C₀-C₃alkyl-O—C₀-C₃alkyl-, —C₀-C₃alkyl-heterocyclyl-C₀-C₅alkyl-N(R³)—C₀-C₃alkyl-, —C₀-C₃alkyl-cycloalkyl-C₀-C₅alkyl-N(R³)—C₀-C₃alkyl-, —C₀-C₃alkyl-O—C₀-C₃alkyl-heterocyclyl-C₀-C₃alkyl-, —C₀-C₃alkyl-O—C₀-C₃alkyl-cycloalkyl-C₀-C₃alkyl-, —C₀-C₃alkyl-heterocyclyl-C₀-C₃alkyl-S—C₀-C₃alkyl-, —C₀-C₃alkyl-cycloalkyl-C₀-C₃alkyl-S—C₀-C₃alkyl-, —C₀-C₃alkyl-N(R³)—C₀-C₃alkyl-heterocyclyl-C₀-C₃alkyl-, —C₀-C₃alkyl-N(R³)—C₀-C₃alkyl-cycloalkyl-C₀-C₃alkyl-, —C₀-C₃alkyl-S—C₀-C₃alkyl-heterocyclyl-C₀-C₃alkyl-, —C₀-C₃alkyl-S—C₀-C₃alkyl-cycloalkyl-C₀-C₃alkyl-, —C₀-C₃alkyl-S(O)₂N(R³)—C₀-C₃alkyl-heterocyclyl-C₀-C₃alkyl-, —C₀-C₃alkyl-S(O)₂N(R³)—C₀-C₃alkyl-cycloalkyl-C₀-C₃alkyl-, —C₀-C₃alkyl-C(O)—N(R³)—C₀-C₃alkyl-heterocyclyl-C₀-C₃alkyl-, —C₀-C₃alkyl-C(O)—N(R³)—C₀-C₃alkyl-cycloalkyl-C₀-C₃alkyl-, —C₀-C₃alkyl-C(S)—N(R³)—C₀-C₃alkyl-heterocyclyl-C₀-C₃alkyl-, —C₀-C₃alkyl-C(S)—N(R³)—C₀-C₃alkyl-cycloalkyl-C₀-C₃alkyl-, —C₀-C₃alkyl-C(O)-heterocyclyl-C₀-C₃alkyl-, —C₀-C₃alkyl-C(O)-cycloalkyl-C₀-C₃alkyl-, —C₀-C₃alkyl-O—C(O)-heterocyclyl-C₀-C₃alkyl-, —C₀-C₃alkyl-O—C(O)-cycloalkyl-C₀-C₃alkyl-, —C₀-C₃alkyl-O—C(O)—N(R³)—C₀-C₃alkyl-heterocyclyl-C₀-C₃alkyl-, —C₀-C₃alkyl-O—C(O)—N(R³)—C₀-C₃alkyl-cycloalkyl-C₀-C₃alkyl-, —C₀-C₃alkyl-O—C(S)—N(R³)—C₀-C₃alkyl-heterocyclyl-C₀-C₃alkyl-, —C₀-C₃alkyl-O—C(S)—N(R³)—C₀-C₃alkyl-cycloalkyl-C₀-C₃alkyl-, —C₀-C₃alkyl-N(R³)—C(O)—N(R³)—C₀-C₃alkyl-heterocyclyl-C₀-C₃alkyl-, —C₀-C₃alkyl-N(R³)—C(O)—N(R³)—C₀-C₃alkyl-cycloalkyl-C₀-C₃alkyl-, —C₀-C₃alkyl-N(R³)—C(S)—N(R³)—C₀-C₃alkyl-heterocyclyl-C₀-C₃alkyl-, —C₀-C₃alkyl-N(R³)—C(S)—N(R³)—C₀-C₃alkyl-cycloalkyl-C₀-C₃alkyl-, —C₀-C₃alkyl-N(R³)—C(O)-heterocyclyl-C₀-C₃alkyl-, —C₀-C₃alkyl-N(R³)—C(O)-cycloalkyl-C₀-C₃alkyl-, —C₀-C₃alkyl-N(R³)—C(S)-heterocyclyl-C₀-C₃alkyl-, —C₀-C₃alkyl-N(R³)—C(S)-cycloalkyl-C₀-C₃alkyl-, —C₀-C₃alkyl-N(R³)—S(O)₂—N(R³)—C₀-C₃alkyl-heterocyclyl-C₀-C₃alkyl-, —C₀-C₃alkyl-N(R³)—S(O)₂—N(R³)—C₀-C₃alkyl-cycloalkyl-C₀-C₃alkyl-, —C₀-C₆alkyl-heterocyclyl-C(O)—C₀-C₆alkyl-, —C₀-C₆alkyl-cycloalkyl-C(O)—C₀-C₆alkyl-, —C₀-C₆alkyl-N(R³)—C(O)-heterocyclyl-C(O)—C₀-C₆alkyl-, —C₀-C₆alkyl-N(R³)—C(O)-cycloalkyl-C(O)—C₀-C₆alkyl-, —C₀-C₆alkyl-N(R³)—C(S)-heterocyclyl-C(O)—C₀-C₆alkyl-, —C₀-C₆alkyl-N(R³)—C(S)-cycloalkyl-C(O)—C₀-C₆alkyl-, —C₀-C₆alkyl-heterocyclyl-S(O)₂—C₀-C₆alkyl-, —C₀-C₆alkyl-cycloalkyl-S(O)₂—C₀-C₆alkyl-, —C₀-C₆alkyl-heterocyclyl-N(R³)—C(O)—C₀-C₆alkyl-, —C₀-C₆alkyl-cycloalkyl-N(R³)—C(O)—C₀-C₆alkyl-, —C₀-C₆alkyl-heterocyclyl-O—C(O)—C₀-C₆alkyl-, —C₀-C₆alkyl-cycloalkyl-O—C(O)—C₀-C₆alkyl-, —C₀-C₆alkyl-heterocyclyl-N(R³)—C(S)—C₀-C₆alkyl-, —C₀-C₆alkyl-cycloalkyl-N(R³)—C(S)—C₀-C₆alkyl-, —C₀-C₆alkyl-heterocyclyl-O—C(S)—C₀-C₆alkyl-, —C₀-C₆alkyl-cycloalkyl-O—C(S)—C₀-C₆alkyl-, —C₀-C₃alkyl-CH═N—O—C₀-C₃alkyl-, —C₀-C₃alkyl-C═N(OH)C(O)—N(R³)—C₂-C₄-alkyl-S—S-alkyl-, —C₀-C₆alkyl-heteroalkyl-C₀-C₆alkyl-C(O)—N(R³)—C₀-C₃alkyl-, —C₀-C₆alkyl-heteroalkyl-C₀-C₆alkyl-C(S)—N(R³)—C₀-C₃alkyl-, —C₀-C₆alkyl-aryl-heteroaryl-C₀-C₆alkyl-, —C₀-C₆alkyl-heteroaryl-aryl-C₀-C₆alkyl-, —C₀-C₆alkyl-heteroaryl-heteroaryl-C₀-C₆alkyl-, —C₀-C₆alkyl-aryl-aryl-C₀-C₆alkyl-, —C₁-C₃alkyl-N(R³)—C(O)—C₁-C₇alkyl-, —C₁-C₃alkyl-N(R³)—C(S)—C₁-C₇alkyl-, —C₀-C₃alkyl-alkenyl-C(O)—C₀-C₆alkyl-, —C₀-C₆alkyl-N(R³)—C(O)—N(R³)—S(O)₀₋₂—C₀-C₃alkyl-, —C₀-C₃alkyl-O-aryl-C₀-C₆ alkyl-N(R³)—C(O)—C₁-C₃ alkyl-, —C₀-C₃alkyl-S-aryl-C₀-C₆ alkyl-N(R³)C(O)—C₁-C₃ alkyl-, —C₀-C₃alkyl-O-aryl-C₀-C₆ alkyl-N(R³)C(S)—C₁-C₃ alkyl-, —C₀-C₃alkyl-S-aryl-C₀-C₆ alkyl-N(R³)C(S)—C₁-C₃ alkyl-, —C₀-C₃alkyl-O-heteroaryl-C₀-C₆ alkyl-N(R³)—C(O)—C₁-C₃alkyl-, —C₀-C₃alkyl-S-heteroaryl-C₀-C₆ alkyl-N(R³)—C(O)—C₁-C₃alkyl-, —C₀-C₃alkyl-O-heteroaryl-C₀-C₆ alkyl-N(R³)—C(S)—C₁-C₃alkyl-, —C₀-C₃alkyl-S-heteroaryl-C₀-C₆ alkyl-N(R³)—C(S)—C₁-C₃alkyl-, —C₀-C₃alkyl-O-heterocyclyl-C₀-C₆ alkyl-N(R³)—C(O)—C₁-C₃alkyl-, —C₀-C₃alkyl-S-heterocyclyl-C₀-C₆ alkyl-N(R³)—C(O)—C₁-C₃alkyl-, —C₀-C₃alkyl-O-heterocyclyl-C₀-C₆ alkyl-N(R³)—C(S)—C₁-C₃alkyl-, —C₀-C₃alkyl-S-heterocyclyl-C₀-C₆ alkyl-N(R³)—C(S)—C₁-C₃alkyl-, —C₀-C₃alkyl-O-cycloalkyl-C₀-C₆alkyl-N(R³)—C(O)—C₁-C₃alkyl-, —C₀-C₃alkyl-S-cycloalkyl-C₀-C₆alkyl-N(R³)—C(O)—C₁-C₃alkyl-, —C₀-C₃alkyl-O-cycloalkyl-C₀-C₆alkyl-N(R³)—C(S)—C₁-C₃alkyl-, —C₀-C₃alkyl-S-cycloalkyl-C₀-C₆alkyl-N(R³)—C(S)—C₁-C₃alkyl-, —C₀-C₃alkyl-N(R³)-aryl-C₀-C₆alkyl-N(R³)—C(O)—C₁-C₃alkyl-, —C₀-C₃alkyl-N(R³)-aryl-C₀-C₆alkyl-N(R³)—C(S)—C₁-C₃alkyl-, —C₀-C₃alkyl-N(R³)-heteroaryl-C₀-C₆alkyl-N(R³)—C(O)—C₁-C₃alkyl-, —C₀-C₃alkyl-N(R³)-heteroaryl-C₀-C₆alkyl-N(R³)—C(S)—C₁-C₃alkyl-, —C₀-C₃alkyl-N(R³)-heterocyclyl-C₀-C₆alkyl-N(R³)—C(O)—C₁-C₃ alkyl-, —C₀-C₃alkyl-N(R³)-heterocyclyl-C₀-C₆alkyl-N(R³)—C(S)—C₁-C₃ alkyl-, —C₀-C₃alkyl-N(R³)-cycloalkyl-C₀-C₆alkyl-N(R³)—C(O)—C₁-C₃alkyl-, —C₀-C₃alkyl-N(R³)-cycloalkyl-C₀-C₆alkyl-N(R³)—C(S)—C₁-C₃alkyl-, —C₀-C₃alkyl-N(R³)—C(O)—O-aryl-C₀-C₆alkyl-N(R³)—C(O)—C₁-C₃alkyl-, —C₀-C₃alkyl-N(R³)—C(S)—O-aryl-C₀-C₆alkyl-N(R³)—C(O)—C₁-C₃alkyl-, —C₀-C₃alkyl-N(R³)—C(O)—S-aryl-C₀-C₆alkyl-N(R³)—C(O)—C₁-C₃alkyl-, —C₀-C₃alkyl-N(R³)—C(O)—O-aryl-C₀-C₆alkyl-N(R³)—C(S)—C₁-C₃alkyl-, —C₀-C₃alkyl-N(R³)—C(S)—O-aryl-C₀-C₆alkyl-N(R³)—C(S)—C₁-C₃alkyl-, —C₀-C₃alkyl-N(R³)—C(O)—S-aryl-C₀-C₆alkyl-N(R³)—C(S)—C₁-C₃alkyl-, —C₀-C₃alkyl-N(R³)—C(O)—O-heteroaryl-C₀-C₆alkyl-N(R³)—C(O)—C₁-C₃alkyl-, —C₀-C₃alkyl-N(R³)—C(S)—O-heteroaryl-C₀-C₆alkyl-N(R³)—C(O)—C₁-C₃alkyl-, —C₀-C₃alkyl-N(R³)—C(O)—S-heteroaryl-C₀-C₆alkyl-N(R³)—C(O)—C₁-C₃alkyl-, —C₀-C₃alkyl-N(R³)—C(O)—O-heteroaryl-C₀-C₆alkyl-N(R³)—C(S)—C₁-C₃alkyl-, —C₀-C₃alkyl-N(R³)—C(S)—O-heteroaryl-C₀-C₆alkyl-N(R³)—C(S)—C₁-C₃alkyl-, —C₀-C₃alkyl-N(R³)—C(O)—O-heterocyclyl-C₀-C₆alkyl-N(R³)—C(O)—C₁-C₃alkyl-, —C₀-C₃alkyl-N(R³)—C(S)—O-heterocyclyl-C₀-C₆alkyl-N(R³)—C(O)—C₁-C₃alkyl-, —C₀-C₃alkyl-N(R³)—C(O)—O-heterocyclyl-C₀-C₆alkyl-N(R³)—C(S)—C₁-C₃alkyl-, —C₀-C₃alkyl-N(R³)—C(S)—O-heterocyclyl-C₀-C₆alkyl-N(R³)—C(S)—C₁-C₃alkyl-, —C₀-C₃alkyl-N(R³)—C(O)—O-cycloalkyl-C₀-C₆alkyl-N(R³)—C(O)—C₁-C₃alkyl-, —C₀-C₃alkyl-N(R³)—C(S)—O-cycloalkyl-C₀-C₆alkyl-N(R³)—C(O)—C₁-C₃alkyl-, —C₀-C₃alkyl-N(R³)—C(O)—O-cycloalkyl-C₀-C₆alkyl-N(R³)—C(S)—C₁-C₃alkyl-, —C₀-C₃alkyl-N(R³)—C(S)—O-cycloalkyl-C₀-C₆alkyl-N(R³)—C(S)—C₁-C₃alkyl-, —C₀-C₃alkyl-C(O)—N(R³)-aryl-C₀-C₆alkyl-N(R³)—C(O)—C₁-C₃alkyl-, —C₀-C₃alkyl-C(S)—N(R³)-aryl-C₀-C₆alkyl-N(R³)—C(O)—C₁-C₃alkyl-, —C₀-C₃alkyl-C(O)—N(R³)-aryl-C₀-C₆alkyl-N(R³)—C(S)—C₁-C₃alkyl-, —C₀-C₃alkyl-C(S)—N(R³)-aryl-C₀-C₆alkyl-N(R³)—C(S)—C₁-C₃alkyl-, —C₀-C₃alkyl-C(O)—N(R³)-heteroaryl-C₀-C₆alkyl-N(R³)C(O)—C₁-C₃alkyl-, —C₀-C₃alkyl-C(S)—N(R³)-heteroaryl-C₀-C₆alkyl-N(R³)C(O)—C₁-C₃alkyl-, —C₀-C₃alkyl-C(O)—N(R³)-heteroaryl-C₀-C₆alkyl-N(R³)C(S)—C₁-C₃alkyl-, —C₀-C₃alkyl-C(S)—N(R³)-heteroaryl-C₀-C₆alkyl-N(R³)C(S)—C₁-C₃alkyl-, —C₀-C₃alkyl-C(O)—N(R³)-heterocyclyl-C₀-C₆alkyl-N(R³)—C(O)—C₁-C₃alkyl-, —C₀-C₃alkyl-C(S)—N(R³)-heterocyclyl-C₀-C₆alkyl-N(R³)—C(O)—C₁-C₃alkyl-, —C₀-C₃alkyl-C(O)—N(R³)-heterocyclyl-C₀-C₆alkyl-N(R³)—C(S)—C₁-C₃alkyl-, —C₀-C₃alkyl-C(S)—N(R³)-heterocyclyl-C₀-C₆alkyl-N(R³)—C(S)—C₁-C₃alkyl-, —C₀-C₃alkyl-C(O)—N(R³)-cycloalkyl-C₀-C₆alkyl-N(R³)—C(O)—C₁-C₃alkyl-, —C₀-C₃alkyl-C(S)—N(R³)-cycloalkyl-C₀-C₆alkyl-N(R³)—C(O)—C₁-C₃alkyl-, —C₀-C₃alkyl-C(O)—N(R³)-cycloalkyl-C₀-C₆alkyl-N(R³)—C(S)—C₁-C₃alkyl-, —C₀-C₃alkyl-C(S)—N(R³)-cycloalkyl-C₀-C₆alkyl-N(R³)—C(S)—C₁-C₃alkyl-, —C₀-C₃alkyl-O—C(O)—N(R³)-aryl-C₀-C₆alkyl-N(R³)—C(O)—C₁-C₃alkyl-, —C₀-C₃alkyl-O—C(S)—N(R³)-aryl-C₀-C₆alkyl-N(R³)—C(O)—C₁-C₃alkyl-, —C₀-C₃alkyl-O—C(O)—N(R³)-aryl-C₀-C₆alkyl-N(R³)—C(S)—C₁-C₃alkyl-, —C₀-C₃alkyl-O—C(S)—N(R³)-aryl-C₀-C₆alkyl-N(R³)—C(S)—C₁-C₃alkyl-, —C₀-C₃alkyl-O—C(O)—N(R³)-heteroaryl-C₀-C₆alkyl-N(R³)—C(O)—C₁-C₃alkyl-, —C₀-C₃alkyl-O—C(S)—N(R³)-heteroaryl-C₀-C₆alkyl-N(R³)—C(O)—C₁-C₃alkyl-, —C₀-C₃alkyl-O—C(O)—N(R³)-heteroaryl-C₀-C₆alkyl-N(R³)—C(S)—C₁-C₃alkyl-, —C₀-C₃alkyl-O—C(S)—N(R³)-heteroaryl-C₀-C₆alkyl-N(R³)—C(S)—C₁-C₃alkyl-, —C₀-C₃alkyl-O—C(O)—N(R³)-heterocyclyl-C₀-C₆alkyl-N(R³)—C(O)—C₁-C₃alkyl-, —C₀-C₃alkyl-O—C(S)—N(R³)-heterocyclyl-C₀-C₆alkyl-N(R³)—C(O)—C₁-C₃alkyl-, —C₀-C₃alkyl-O—C(O)—N(R³)-heterocylcyl-C₀-C₆alkyl-N(R³)—C(S)—C₁-C₃alkyl-, —CO—C₃alkyl-O—C(S)—N(R³)-heterocyclyl-C₀-C₆alkyl-N(R³)—C(S)—C₁-C₃alkyl-, —C₀-C₃alkyl-O—C(O)—N(R³)-cycloalkyl-C₀-C₆alkyl-N(R³)—C(O)—C₁-C₃alkyl-, —C₀-C₃alkyl-O—C(S)—N(R³)-cycloalkyl-C₀-C₆alkyl-N(R³)—C(O)—C₁-C₃alkyl-, —C₀-C₃alkyl-O—C(O)—N(R³)-cycloalkyl-C₀-C₆alkyl-N(R³)—C(S)—C₁-C₃alkyl-, —C₀-C₃alkyl-O—C(S)—N(R³)-cycloalkyl-C₀-C₆alkyl-N(R³)—C(S)—C₁-C₃alkyl-, —C₀-C₃alkyl-S(O)₂—N(R³)-aryl-C₀-C₆alkyl-N(R³)—C(O)—C₁-C₃alkyl-, —C₀-C₃alkyl-S(O)₂—N(R³)-aryl-C₀-C₆alkyl-N(R³)—C(S)—C₁-C₃alkyl-, —C₀-C₃ alkyl-S(O)₂—N(R³)-heteroaryl-C₀-C₆alkyl-N(R³)—C(O)—C₁-C₃alkyl-, —C₀-C₃ alkyl-S(O)₂—N(R³)-heteroaryl-C₀-C₆alkyl-N(R³)—C(S)—C₁-C₃alkyl-, —C₀-C₃alkyl-S(O)₂—N(R³)-heterocyclyl-C₀-C₆alkyl-N(R³)—C(O)—C₁-C₃alkyl-, —C₀-C₃alkyl-S(O)₂—N(R³)-heterocyclyl-C₀-C₆alkyl-N(R³)—C(S)—C₁-C₃alkyl-, —C₀-C₃alkyl-S(O)₂—N(R³)-cycloalkyl-C₀-C₆alkyl-N(R³)—C(O)—C₁-C₃alkyl-, —C₀-C₃alkyl-S(O)₂—N(R³)-cycloalkyl-C₀-C₆alkyl-N(R³)—C(S)—C₁-C₃alkyl-, —C₀-C₃alkyl-N(R³)—S(O)₂—N(R³)-aryl-C₀-C₆alkyl-N(R³)—C(O)—C₁-C₃alkyl-, —C₀-C₃alkyl-N(R³)—S(O)₂—N(R³)-aryl-C₀-C₆alkyl-N(R³)—C(S)—C₁-C₃alkyl-, —C₀-C₃alkyl-N(R³)—S(O)₂—N(R³)-heteroaryl-C₀-C₆alkyl-N(R³)—C(O)—C₁-C₃alkyl-, —C₀-C₃alkyl-N(R³)—S(O)₂—N(R³)-heteroaryl-C₀-C₆alkyl-N(R³)—C(S)—C₁-C₃alkyl-, —C₀-C₃alkyl-N(R³)—S(O)₂—N(R³)-heterocyclyl-C₀-C₆alkyl-N(R³)—C(O)—C₁-C₃alkyl-, —C₀-C₃alkyl-N(R³)—S(O)₂—N(R³)-heterocyclyl-C₀-C₆alkyl-N(R³)—C(S)—C₁-C₃alkyl-, —C₀-C₃alkyl-N(R³)— S(O)₂—N(R³)-cycloalkyl-C₀-C₆alkyl-N(R³)—C(O)—C₁-C₃alkyl-, —C₀-C₃alkyl-N(R³)— S(O)₂—N(R³)-cycloalkyl-C₀-C₆alkyl-N(R³)—C(S)—C₁-C₃alkyl-, —C₀-C₃alkyl-heterocyclyl-O-aryl-C₀-C₆alkyl-N(R³)—C(O)—C₁-C₃alkyl-, —C₀-C₃alkyl-heterocyclyl-S-aryl-C₀-C₆alkyl-N(R³)—C(O)—C₁-C₃alkyl-, —C₀-C₃alkyl-heterocyclyl-O-aryl-C₀-C₆alkyl-N(R³)—C(S)—C₁-C₃alkyl-, —C₀-C₃alkyl-heterocyclyl-S-aryl-C₀-C₆alkyl-N(R³)—C(S)—C₁-C₃alkyl-, —C₀-C₃alkyl-heterocyclyl-O-heteroaryl-C₀-C₆alkyl-N(R³)—C(O)—C₁-C₃alkyl-, —C₀-C₃alkyl-heterocyclyl-S-heteroaryl-C₀-C₆alkyl-N(R³)—C(O)—C₁-C₃alkyl-, —C₀-C₃alkyl-heterocyclyl-O-heteroaryl-C₀-C₆alkyl-N(R³)—C(S)—C₁-C₃alkyl-, —C₀-C₃alkyl-heterocyclyl-S-heteroaryl-C₀-C₆alkyl-N(R³)—C(S)—C₁-C₃alkyl-, —C₀-C₃alkyl-heterocyclyl-O-heterocyclyl-C₀-C₆alkyl-N(R³)—C(O)—C₁-C₃alkyl-, —C₀-C₃alkyl-heterocyclyl-S-heterocyclyl-C₀-C₆alkyl-N(R³)—C(O)—C₁-C₃alkyl-, —C₀-C₃alkyl-heterocyclyl-O-heterocyclyl-C₀-C₆alkyl-N(R³)—C(S)—C₁-C₃alkyl-, —C₀-C₃alkyl-heterocyclyl-S-heterocyclyl-C₀-C₆alkyl-N(R³)—C(S)—C₁-C₃alkyl-, —C₀-C₃alkyl-heterocyclyl-O-cycloalkyl-C₀-C₆alkyl-N(R³)—C(O)—C₁-C₃alkyl-, —C₀-C₃alkyl-heterocyclyl-S-cycloalkyl-C₀-C₆alkyl-N(R³)—C(O)—C₁-C₃alkyl-, —C₀-C₃alkyl-heterocyclyl-O-cycloalkyl-C₀-C₆alkyl-N(R³)—C(S)—C₁-C₃alkyl-, —C₀-C₃alkyl-heterocyclyl-S-cycloalkyl-C₀-C₆alkyl-N(R³)—C(S)—C₁-C₃alkyl-, —C₀-C₃alkyl-C(O)-heterocyclyl-O-aryl-C₀-C₆alkyl-N(R³)—C(O)—C₁-C₃alkyl-, —C₀-C₃alkyl-C(S)-heterocyclyl-O-aryl-C₀-C₆alkyl-N(R³)—C(O)—C₁-C₃alkyl-, —C₀-C₃alkyl-C(O)-heterocyclyl-S-aryl-C₀-C₆alkyl-N(R³)—C(O)—C₁-C₃alkyl-, —C₀-C₃alkyl-C(O)-heterocyclyl-O-aryl-C₀-C₆alkyl-N(R³)—C(S)—C₁-C₃alkyl-, —C₀-C₃alkyl-C(S)-heterocyclyl-S-aryl-C₀-C₆alkyl-N(R³)—C(O)—C₁-C₃alkyl-, —C₀-C₃alkyl-C(S)-heterocyclyl-O-aryl-C₀-C₆alkyl-N(R³)—C(S)—C₁-C₃alkyl-, —C₀-C₃alkyl-C(O)-heterocyclyl-S-aryl-C₀-C₆alkyl-N(R³)—C(S)—C₁-C₃alkyl-, —C₀-C₃alkyl-C(S)-heterocyclyl-S-aryl-C₀-C₆alkyl-N(R³)—C(S)—C₁-C₃alkyl-, —C₀-C₃alkyl-C(O)-heterocyclyl-O-heteroaryl-C₀-C₆alkyl-N(R³)—C(O)—C₁-C₃alkyl-, —C₀-C₃alkyl-C(S)-heterocyclyl-O-heteroaryl-C₀-C₆alkyl-N(R³)—C(O)—C₁-C₃alkyl-, —C₀-C₃alkyl-C(O)-heterocyclyl-5-heteroaryl-C₀-C₆alkyl-N(R³)—C(O)—C₁-C₃alkyl-, —C₀-C₃alkyl-C(O)-heterocyclyl-O-heteroaryl-C₀-C₆alkyl-N(R³)—C(S)—C₁-C₃alkyl-, —C₀-C₃alkyl-C(S)-heterocyclyl-S-heteroaryl-C₀-C₆alkyl-N(R³)—C(O)—C₁-C₃alkyl-, —C₀-C₃alkyl-C(S)-heterocyclyl-O-heteroaryl-C₀-C₆alkyl-N(R³)—C(S)—C₁-C₃alkyl-, —C₀-C₃alkyl-C(O)-heterocyclyl-S-heteroaryl-C₀-C₆alkyl-N(R³)—C(S)—C₁-C₃alkyl-, —C₀-C₃alkyl-C(S)-heterocyclyl-S-heteroaryl-C₀-C₆alkyl-N(R³)—C(S)—C₁-C₃alkyl-, —C₀-C₃alkyl-C(O)-heterocyclyl-O-heterocyclyl-C₀-C₆alkyl-N(R³)—C(O)—C₁-C₃alkyl-, —C₀-C₃alkyl-C(S)-heterocyclyl-O-heterocyclyl-C₀-C₆alkyl-N(R³)—C(O)—C₁-C₃alkyl-, —C₀-C₃alkyl-C(O)-heterocyclyl-S-heterocyclyl-C₀-C₆alkyl-N(R³)—C(O)—C₁-C₃alkyl-, —C₀-C₃alkyl-C(O)-heterocyclyl-O-heterocyclyl-C₀-C₆alkyl-N(R³)—C(S)—C₁-C₃alkyl-, —C₀-C₃alkyl-C(S)-heterocyclyl-S-heterocyclyl-C₀-C₆alkyl-N(R³)—C(O)—C₁-C₃alkyl-, —C₀-C₃alkyl-C(S)-heterocyclyl-O-heterocyclyl-C₀-C₆alkyl-N(R³)—C(S)—C₁-C₃alkyl-, —C₀-C₃alkyl-C(O)-heterocyclyl-S-heterocyclyl-C₀-C₆alkyl-N(R³)—C(S)—C₁-C₃alkyl-, —C₀-C₃alkyl-C(S)-heterocyclyl-S-heterocycyl-C₀-C₆alkyl-N(R³)—C(S)—C₁-C₃alkyl-, —CO—C₃alkyl-C(O)-heterocyclyl-O-cycloalkyl-C₀-C₆alkyl-N(R³)—C(O)—C₁-C₃alkyl-, —C₀-C₃alkyl-C(S)-heterocyclyl-O-cycloalkyl-C₀-C₆alkyl-N(R³)—C(O)—C₁-C₃alkyl-, —C₀-C₃alkyl-C(O)-heterocyclyl-S-cycloalkyl-C₀-C₆alkyl-N(R³)—C(O)—C₁-C₃alkyl-, —C₀-C₃alkyl-C(O)-heterocyclyl-O-cycloalkyl-C₀-C₆alkyl-N(R³)—C(S)—C₁-C₃alkyl-, —C₀-C₃alkyl-C(S)-heterocyclyl-S-cycloalkyl-C₀-C₆alkyl-N(R³)—C(O)—C₁-C₃alkyl-, —C₀-C₃alkyl-C(S)-heterocyclyl-O-cycloalkyl-C₀-C₆alkyl-N(R³)—C(S)—C₁-C₃alkyl-, —C₀-C₃alkyl-C(O)-heterocyclyl-S-cycloalkyl-C₀-C₆alkyl-N(R³)—C(S)—C₁-C₃alkyl-, —C₀-C₃alkyl-C(S)-heterocyclyl-S-cycloalkyl-C₀-C₆alkyl-N(R³)—C(S)—C₁-C₃alkyl-, —C₀-C₃alkyl-N(R³)—C(O)-heterocyclyl-O-aryl-C₀-C₆alkyl-N(R³)—C(O)—C₁-C₃alkyl-, —C₀-C₃alkyl-N(R³)—C(S)-heterocyclyl-O-aryl-C₀-C₆alkyl-N(R³)—C(O)—C₁-C₃alkyl-, —C₀-C₃alkyl-N(R³)—C(O)-heterocyclyl-S-aryl-C₀-C₆alkyl-N(R³)—C(O)—C₁-C₃alkyl-, —C₀-C₃alkyl-N(R³)—C(O)-heterocyclyl-O-aryl-C₀-C₆alkyl-N(R³)—C(S)—C₁-C₃alkyl-, —C₀-C₃alkyl-N(R³)—C(S)-heterocyclyl-S-aryl-C₀-C₆alkyl-N(R³)—C(O)—C₁-C₃alkyl-, —CO—C₃alkyl-N(R³)—C(S)-heterocyclyl-O-aryl-C₀-C₆alkyl-N(R³)—C(S)—C₁-C₃alkyl-, —C₀-C₃alkyl-N(R³)—C(O)-heterocyclyl-S-aryl-C₀-C₆alkyl-N(R³)—C(S)—C₁-C₃alkyl-, —C₀-C₃alkyl-N(R³)—C(S)-heterocyclyl-S-aryl-C₀-C₆alkyl-N(R³)—C(S)—C₁-C₃alkyl-, —C₀-C₃alkyl-N(R³)—C(O)-heterocyclyl-O-heteroaryl-C₀-C₆alkyl-N(R³)—C(O)—C₁-C₃alkyl-, —C₀-C₃alkyl-N(R³)—C(S)-heterocyclyl-O-heteroaryl-C₀-C₆alkyl-N(R³)—C(O)—C₁-C₃alkyl-, —C₀-C₃alkyl-N(R³)—C(O)-heterocyclyl-S-heteroaryl-C₀-C₆alkyl-N(R³)—C(O)—C₁-C₃alkyl-, —C₀-C₃alkyl-N(R³)—C(O)-heterocyclyl-O-heteroaryl-C₀-C₆alkyl-N(R³)—C(S)—C₁-C₃alkyl-, —C₀-C₃alkyl-N(R³)—C(S)-heterocyclyl-S-heteroaryl-C₀-C₆alkyl-N(R³)—C(O)—C₁-C₃alkyl-, —C₀-C₃alkyl-N(R³)—C(S)-heterocyclyl-O-heteroaryl-C₀-C₆alkyl-N(R³)—C(S)—C₁-C₃alkyl-, —C₀-C₃alkyl-N(R³)—C(O)-heterocyclyl-S-heteroaryl-C₀-C₆alkyl-N(R³)—C(S)—C₁-C₃alkyl-, —C₀-C₃alkyl-N(R³)—C(S)-heterocyclyl-S-heteroaryl-C₀-C₆alkyl-N(R³)—C(S)—C₁-C₃alkyl-, —C₀-C₃alkyl-N(R³)—C(O)-heterocyclyl-O-heterocyclyl-C₀-C₆alkyl-N(R³)—C(O)—C₁-C₃alkyl-, —C₀-C₃alkyl-N(R³)—C(S)-heterocyclyl-O-heterocyclyl-C₀-C₆alkyl-N(R³)—C(O)—C₁-C₃alkyl-, —C₀-C₃alkyl-N(R³)—C(O)-heterocyclyl-S-heterocyclyl-C₀-C₆alkyl-N(R³)—C(O)—C₁-C₃alkyl-, —C₀-C₃alkyl-N(R³)—C(O)-heterocyclyl-O-heterocyclyl-C₀-C₆alkyl-N(R³)—C(S)—C₁-C₃alkyl-, —C₀-C₃alkyl-N(R³)—C(S)-heterocyclyl-S-heterocyclyl-C₀-C₆alkyl-N(R³)—C(O)—C₁-C₃alkyl-, —C₀-C₃alkyl-N(R³)—C(S)-heterocyclyl-O-heterocyclyl-C₀-C₆alkyl-N(R³)—C(S)—C₁-C₃alkyl-, —C₀-C₃alkyl-N(R³)—C(O)-heterocyclyl-S-heterocyclyl-C₀-C₆alkyl-N(R³)—C(S)—C₁-C₃alkyl-, —C₀-C₃alkyl-N(R³)—C(S)-heterocyclyl-S-heterocyclyl-C₀-C₆alkyl-N(R³)—C(S)—C₁-C₃alkyl-, —C₀-C₃alkyl-N(R³)—C(O)-heterocyclyl-O-cycloalkyl-C₀-C₆alkyl-N(R³)—C(O)—C₁-C₃alkyl-, —C₀-C₃alkyl-N(R³)—C(S)-heterocyclyl-O-cycloalkyl-C₀-C₆alkyl-N(R³)—C(O)—C₁-C₃alkyl-, —C₀-C₃alkyl-N(R³)—C(O)-heterocyclyl-S-cycloalkyl-C₀-C₆alkyl-N(R³)—C(O)—C₁-C₃alkyl-, —C₀-C₃alkyl-N(R³)—C(O)-heterocyclyl-O-cycloalkyl-C₀-C₆alkyl-N(R³)C(S)—C₁-C₃alkyl-, —C₀-C₃alkyl-N(R³)—C(S)-heterocyclyl-S-cycloalkyl-C₀-C₆alkyl-N(R³)—C(O)—C₁-C₃alkyl-, —C₀-C₃alkyl-N(R³)—C(S)-heterocyclyl-O-cycloalkyl-C₀-C₆alkyl-N(R³)C(S)—C₁-C₃alkyl-, —C₀-C₃alkyl-N(R³)—C(O)-heterocyclyl-S-cycloalkyl-C₀-C₆alkyl-N(R³)—C(S)—C₁-C₃alkyl-, —C₀-C₃alkyl-N(R³)—C(S)-heterocyclyl-S-cycloalkyl-C₀-C₆alkyl-N(R³)—C(S)—C₁-C₃alkyl-, —C₀-C₃alkyl-S(O)₂-heterocyclyl-O-aryl-C₀-C₆alkyl-N(R³)—C(O)—C₁-C₃alkyl-, —C₀-C₃alkyl-S(O)₂-heterocyclyl-S-aryl-C₀-C₆alkyl-N(R³)—C(O)—C₁-C₃alkyl-, —C₀-C₃alkyl-S(O)₂-heterocyclyl-O-aryl-C₀-C₆alkyl-N(R³)C(S)—C₁-C₃alkyl-, —CO—C₃alkyl-S(O)₂-heterocyclyl-S-aryl-C₀-C₆alkyl-N(R³)—C(S)—C₁-C₃alkyl-, —C₀-C₃alkyl-S(O)₂-heterocyclyl-O-heteroaryl-C₀-C₆alkyl-N(R³)—C(O)—C₁-C₃alkyl-, —C₀-C₃alkyl-S(O)₂-heterocyclyl-S-heteroaryl-C₀-C₆alkyl-N(R³)—C(O)—C₁-C₃alkyl-, —C₀-C₃alkyl-S(O)₂-heterocyclyl-O-heteroaryl-C₀-C₆alkyl-N(R³)—C(S)—C₁-C₃alkyl-, —C₀-C₃alkyl-S(O)₂-heterocyclyl-S-heteroaryl-C₀-C₆alkyl-N(R³)—C(S)—C₁-C₃alkyl-, —C₀-C₃alkyl-S(O)₂-heterocyclyl-O-heterocyclyl-C₀-C₆alkyl-N(R³)—C(O)—C₁-C₃alkyl-, —C₀-C₃alkyl-S(O)₂-heterocyclyl-S-heterocyclyl-C₀-C₆alkyl-N(R³)C(O)—C₁-C₃alkyl-, —C₀-C₃alkyl-S(O)₂-heterocyclyl-O-heterocyclyl-C₀-C₆alkyl-N(R³)C(S)—C₁-C₃alkyl-, —C₀-C₃alkyl-S(O)₂-heterocyclyl-S-heterocyclyl-C₀-C₆alkyl-N(R³)—C(S)—C₁-C₃alkyl-, —C₀-C₃alkyl-S(O)₂-heterocyclyl-O-cycloalkyl-C₀-C₆alkyl-N(R³)—C(O)—C₁-C₃alkyl-, —C₀-C₃alkyl-S(O)₂-heterocyclyl-S-cycloalkyl-C₀-C₆alkyl-N(R³)—C(O)—C₁-C₃alkyl-, —C₀-C₃alkyl-S(O)₂-heterocyclyl-O-cycloalkyl-C₀-C₆alkyl-N(R³)—C(S)—C₁-C₃alkyl-, —C₀-C₃alkyl-S(O)₂-heterocyclyl-S-cycloalkyl-C₀-C₆alkyl-N(R³)—C(S)—C₁-C₃alkyl-, —C₀-C₃alkyl-heterocyclyl-N(R³)-aryl-C₀-C₆alkyl-N(R³)C(O)—C₁-C₃alkyl-, —C₀-C₃alkyl-heterocyclyl-N(R³)-aryl-C₀-C₆alkyl-N(R³)—C(S)—C₁-C₃alkyl-, —C₀-C₃alkyl-heterocyclyl-N(R³)-heteroaryl-C₀-C₆alkyl-N(R³)—C(O)—C₁-C₃alkyl-, —C₀-C₃alkyl-heterocyclyl-N(R³)-heteroaryl-C₀-C₆alkyl-N(R³)—C(S)—C₁-C₃alkyl-, —C₀-C₃alkyl-heterocyclyl-N(R³)-heterocyclyl-C₀-C₆alkyl-N(R³)—C(O)—C₁-C₃alkyl-, —C₀-C₃alkyl-heterocyclyl-N(R³)-heterocyclyl-C₀-C₆alkyl-N(R³)—C(S)—C₁-C₃alkyl-, —C₀-C₃alkyl-heterocyclyl-N(R³)-cycloalkyl-C₀-C₆alkyl-N(R³)—C(O)—C₁-C₃alkyl-, —C₀-C₃alkyl-heterocyclyl-N(R³)-cycloalkyl-C₀-C₆alkyl-N(R³)—C(S)—C₁-C₃alkyl-, —C₀-C₃alkyl-C(O)-heterocyclyl-N(R³)-aryl-C₀-C₆alkyl-N(R³)—C(O)—C₁-C₃alkyl-, —C₀-C₃alkyl-C(S)-heterocyclyl-N(R³)-aryl-C₀-C₆alkyl-N(R³)—C(O)—C₁-C₃alkyl-, —C₀-C₃alkyl-C(O)-heterocyclyl-N(R³)-aryl-C₀-C₆alkyl-N(R³)—C(S)—C₁-C₃alkyl-, —C₀-C₃alkyl-C(S)-heterocyclyl-N(R³)-aryl-C₀-C₆alkyl-N(R³)—C(S)—C₁-C₃alkyl-, —C₀-C₃alkyl-C(O)-heterocyclyl-N(R³)-heteroaryl-C₀-C₆alkyl-N(R³)—C(O)—C₁-C₃alkyl-, —C₀-C₃alkyl-C(S)-heterocyclyl-N(R³)-heteroaryl-C₀-C₆alkyl-N(R³)—C(O)—C₁-C₃alkyl-, —C₀-C₃alkyl-C(O)-heterocyclyl-N(R³)-heteroaryl-C₀-C₆alkyl-N(R³)—C(S)—C₁-C₃alkyl-, —C₀-C₃alkyl-C(S)-heterocyclyl-N(R³)-heteroaryl-C₀-C₆alkyl-N(R³)—C(S)—C₁-C₃alkyl-, —C₀-C₃alkyl-C(O)-heterocyclyl-N(R³)-heterocyclyl-C₀-C₆alkyl-N(R³)—C(O)—C₁-C₃alkyl-, —C₀-C₃alkyl-C(S)-heterocyclyl-N(R³)-heterocyclyl-C₀-C₆alkyl-N(R³)—C(O)—C₁-C₃alkyl-, —C₀-C₃alkyl-C(O)-heterocyclyl-N(R³)-heterocyclyl-C₀-C₆alkyl-N(R³)—C(S)—C₁-C₃alkyl-, —C₀-C₃alkyl-C(S)-heterocyclyl-N(R³)-heterocyclyl-C₀-C₆alkyl-N(R³)—C(S)—C₁-C₃alkyl-, —C₀-C₃alkyl-C(O)-heterocyclyl-N(R³)-cycloalkyl-C₀-C₆alkyl-N(R³)—C(O)—C₁-C₃alkyl-, —C₀-C₃alkyl-C(S)-heterocyclyl-N(R³)-cycloalkyl-C₀-C₆alkyl-N(R³)—C(O)—C₁-C₃alkyl-, —C₀-C₃alkyl-C(O)-heterocyclyl-N(R³)-cycloalkyl-C₀-C₆alkyl-N(R³)—C(S)—C₁-C₃alkyl-, —C₀-C₃alkyl-C(S)-heterocyclyl-N(R³)-cycloalkyl-C₀-C₆alkyl-N(R³)—C(S)—C₁-C₃alkyl-, —C₀-C₃alkyl-N(R³)—C(O)-heterocyclyl-N(R³)-aryl-C₀-C₆alkyl-N(R³)—C(O)—C₁-C₃alkyl-, —C₀-C₃alkyl-N(R³)—C(S)-heterocyclyl-N(R³)-aryl-C₀-C₆alkyl-N(R³)—C(O)—C₁-C₃alkyl-, —C₀-C₃alkyl-N(R³)—C(O)-heterocyclyl-N(R³)-aryl-C₀-C₆alkyl-N(R³)—C(S)—C₁-C₃alkyl-, —C₀-C₃alkyl-N(R³)—C(S)-heterocyclyl-N(R³)-aryl-C₀-C₆alkyl-N(R³)—C(S)—C₁-C₃alkyl-, —C₀-C₃alkyl-N(R³)—C(O)-heterocyclyl-N(R³)-heteroaryl-C₀-C₆alkyl-N(R³)—C(O)—C₁-C₃alkyl-, —C₀-C₃alkyl-N(R³)—C(S)-heterocyclyl-N(R³)-heteroaryl-C₀-C₆alkyl-N(R³)—C(O)—C₁-C₃alkyl-, —C₀-C₃alkyl-N(R³)—C(O)-heterocyclyl-N(R³)-heteroaryl-C₀-C₆alkyl-N(R³)—C(S)—C₁-C₃alkyl-, —C₀-C₃alkyl-N(R³)—C(S)-heterocyclyl-N(R³)-heteroaryl-C₀-C₆alkyl-N(R³)—C(S)—C₁-C₃alkyl-, —C₀-C₃alkyl-N(R³)—C(O)-heterocyclyl-N(R³)-heterocyclyl-C₀-C₆alkyl-N(R³)—C(O)—C₁-C₃alkyl-, —C₀-C₃alkyl-N(R³)—C(S)-heterocyclyl-N(R³)-heterocyclyl-C₀-C₆alkyl-N(R³)—C(O)—C₁-C₃alkyl-, —C₀-C₃alkyl-N(R³)—C(O)-heterocyclyl-N(R³)-heterocyclyl-C₀-C₆alkyl-N(R³)—C(S)—C₁-C₃alkyl-, —C₀-C₃alkyl-N(R³)—C(S)-heterocyclyl-N(R³)-heterocyclyl-C₀-C₆alkyl-N(R³)—C(S)—C₁-C₃alkyl-, —C₀-C₃alkyl-N(R³)—C(O)-heterocyclyl-N(R³)-cycloalkyl-C₀-C₆alkyl-N(R³)—C(O)—C₁-C₃alkyl-, —C₀-C₃alkyl-N(R³)—C(S)-heterocyclyl-N(R³)-cycloalkyl-C₀-C₆alkyl-N(R³)—C(O)—C₁-C₃alkyl-, —C₀-C₃alkyl-N(R³)—C(O)-heterocyclyl-N(R³)-cycloalkyl-C₀-C₆alkyl-N(R³)—C(S)—C₁-C₃alkyl-, —C₀-C₃alkyl-N(R³)—C(S)-heterocyclyl-N(R³)-cycloalkyl-C₀-C₆alkyl-N(R³)—C(S)—C₁-C₃alkyl-, —C₀-C₃alkyl-S(O)₂-heterocyclyl-N(R³)-aryl-C₀-C₆alkyl-N(R³)—C(O)—C₁-C₃alkyl-, —CO—C₃alkyl-S(O)₂-heterocyclyl-N(R³)-aryl-C₀-C₆alkyl-N(R³)—C(S)—C₁-C₃alkyl-, —C₀-C₃alkyl-S(O)₂-heterocyclyl-N(R³)-heteroaryl-C₀-C₆alkyl-N(R³)—C(O)—C₁-C₃alkyl-, —C₀-C₃alkyl-S(O)₂-heterocyclyl-N(R³)-heteroaryl-C₀-C₆alkyl-N(R³)—C(S)—C₁-C₃alkyl-, —C₀-C₃alkyl-S(O)₂-heterocyclyl-N(R³)-heterocyclyl-C₀-C₆alkyl-N(R³)—C(O)—C₁-C₃alkyl-, —C₀-C₃alkyl-S(O)₂-heterocyclyl-N(R³)-heterocyclyl-C₀-C₆alkyl-N(R³)—C(S)—C₁-C₃alkyl-, —C₀-C₃alkyl-S(O)₂-heterocyclyl-N(R³)-cycloalkyl-C₀-C₆alkyl-N(R³)—C(O)—C₁-C₃alkyl-, —C₀-C₃alkyl-S(O)₂-heterocyclyl-N(R³)-cycloalkyl-C₀-C₆alkyl-N(R³)—C(S)—C₁-C₃alkyl-, —C₀-C₆alkyl-N(R³)—C(O)—C₀-C₆alkyl-heterocycloalkyl-C(O)—C₀-C₃alkyl-, —C₀-C₆alkyl-N(R³)—C(S)—C₀-C₆alkyl-heterocycloalkyl-C(O)—C₀-C₃alkyl-, —C₀-C₆alkyl-N(R³)—C(O)—C₀-C₆alkyl-heterocycloalkyl-C(S)—C₀-C₃alkyl-, —C₀-C₆alkyl-N(R³)—C(S)—C₀-C₆alkyl-heterocycloalkyl-C(S)—C₀-C₃alkyl-, —C₀-C₆alkyl-N(R³)—C(O)—C₀-C₆alkyl-heterocycloalkyl-C(O)—N(R³)—C₀-C₃alkyl-, —C₀-C₆alkyl-N(R³)—C(S)—C₀-C₆alkyl-heterocycloalkyl-C(O)—N(R³)—C₀-C₃alkyl-, —C₀-C₆alkyl-N(R³)—C(O)—C₀-C₆alkyl-heterocycloalkyl-C(S)—N(R³)—C₀-C₃alkyl-, —C₀-C₆alkyl-N(R³)—C(S)—C₀-C₆alkyl-heterocycloalkyl-C(S)—N(R³)—C₀-C₃alkyl-, —C₀-C₆alkyl-C(O)—C₀-C₆alkyl-heterocycloalkyl-C(O)—N(R³)—C₀-C₃alkyl-, —C₀-C₆alkyl-C(S)—C₀-C₆alkyl-heterocycloalkyl-C(O)—N(R³)—C₀-C₃alkyl-, —C₀-C₆alkyl-C(O)—C₀-C₆alkyl-heterocycloalkyl-C(S)—N(R³)—C₀-C₃alkyl-, —C₀-C₆alkyl-C(S)—C₀-C₆alkyl-heterocycloalkyl-C(S)—N(R³)—C₀-C₃alkyl-, —C₀-C₆alkyl-O—C₀-C₃alkyl-C(O)—N(R³)—C₀-C₃alkyl- and —C₀-C₆alkyl-O—C(S)—N(R³)—C₀-C₃alkyl-, wherein each alkyl, alkenyl, alkynyl, heteroalkyl, cycloalkyl, heterocycloalkyl, heterocyclyl, aryl and heteroaryl moiety of the aforementioned L are optionally substituted; wherein
Y is selected from the group consisting of H, alkyl, heteroalkyl, cycloalkyl, heterocyclyl, cycloalkylalkyl, heterocyclylalkyl, aryl, arylalkyl, heteroaryl, heteroarylalkyl, aryl-heteroaryl, aryl-heteroarylalkyl, heteroaryl-alkylaryl, aryl-aryl, aryl-arylalkyl, aryl-alkylaryl, aryl-C₀-C₃alkyl-O—C₀-C₃alkyl-aryl, aryl-C₀-C₃alkyl-S(O)₀₋₂—C₀-C₃alkyl-aryl, —C₀-C₃alkyl-S(O)₀₋₂—C₀-C₃alkyl-aryl, aryl-C₀-C₃alkyl-N(R³)—C₀-C₃alkyl-aryl, aryl-C₀-C₃alkyl-C(O)—N(R³)—C₀-C₃alkyl-aryl, aryl-C₀-C₃alkyl-C(S)—N(R³)—C₀-C₃alkyl-aryl, aryl-C₀-C₃alkyl-N(R³)—C(O)—C₀-C₃alkyl-aryl, aryl-C₀-C₃alkyl-N(R³)—C(S)—C₀-C₃alkyl-aryl, heteroaryl-heteroaryl, heteroaryl-aryl, heteroaryl-arylalkyl, aryl-alkylheteroaryl, heteroaryl-aryl-aryl, aryl-aryl-aryl, aryl-heteroaryl-aryl, aryl-heteroaryl-heteroaryl, heteroaryl-heteroaryl-heteroaryl, heteroaryl-heteroaryl-aryl, aryl-aryl-heteroaryl, heteroaryl-aryl-arylalkyl, aryl-aryl-alkylheteroaryl, heteroaryl-aryl-alkylaryl, aryl-aryl-alkylaryl, aryl-aryl-arylalkyl, aryl-aryl-heteroarylalkyl, heteroaryl-aryl-heteroaryl, heteroaryl-aryl-heteroarylalkyl, heteroaryl-aryl-alkylheteroaryl, heteroaryl-heteroarylalkyl, heteroaryl-alkylheteroaryl, heterocyclyl-heteroaryl, cycloalkyl-aryl, cycloalkyl-heteroaryl, heteroaryl-heterocyclyl, heteroaryl-cycloalkyl, aryl-cycloalkyl, heterocyclyl-aryl, aryl-heterocyclyl, heterocyclyl-alkyl-aryl, heterocyclyl-alkylheteroaryl, cycloalkyl-alkylaryl, cycloalkyl-alkylheteroaryl, aryl-alkyl-heterocyclyl, aryl-alkylcycloalkyl, heteroaryl-alkylcycloalkyl, heteroaryl-alkylheterocyclyl, arylalkyl-aryl, aryl-arylalkyl, aryl-heteroarylalkyl, heteroaryl-arylalkyl, heteroaryl-heteroarylalkyl, heteroaryl-C₀-C₃alkyl-O—C₀-C₃alkyl-aryl, heteroaryl-C₀-C₃alkyl-O—C₀-C₃alkyl-heteroaryl, aryl-C₀-C₃alkyl-O—C₀-C₃alkyl-heteroaryl, heteroaryl-C₀-C₃alkyl-N(R³)—C₀-C₃alkyl-aryl, aryl-C₀-C₃alkyl-N(R³)—C₀-C₃alkyl-heteroaryl, heteroaryl-C₀-C₃alkyl-N(R³)—C₀-C₃alkyl-heteroaryl, heteroaryl-C₀-C₃alkyl-C(O)—N(R³)—C₀-C₃alkyl-aryl, aryl-C₀-C₃alkyl-C(O)—N(R³)—C₀-C₃alkyl-heteroaryl, heteroaryl-C₀-C₃alkyl-C(O)—N(R³)—C₀-C₃alkyl-heteroaryl, aryl-C₀-C₃alkyl-C(S)—N(R³)—C₀-C₃alkyl-aryl, aryl-C₀-C₃alkyl-C(S)—N(R³)—C₀-C₃alkyl-heteroaryl, heteroaryl-C₀-C₃alkyl-C(S)—N(R³)—C₀-C₃alkyl-aryl, heteroaryl-C₀-C₃alkyl-C(S)—N(R³)—C₀-C₃alkyl-heteroaryl, heteroaryl-C₀-C₃alkyl-N(R³)—C(O)—C₀-C₃alkyl-aryl, heteroaryl-C₀-C₃alkyl-N(R³)—C(O)—C₀-C₃alkyl-heteroaryl, aryl-C₀-C₃alkyl-N(R³)—C(O)—C₀-C₃alkyl-heteroaryl, aryl-C₀-C₃alkyl-N(R³)—C(S)—C₀-C₃alkyl-aryl, aryl-C₀-C₃alkyl-N(R³)—C(S)—C₀-C₃alkyl-heteroaryl, heteroaryl-C₀-C₃alkyl-N(R³)—C(S)—C₀-C₃alkyl-aryl, heteroaryl-C₀-C₃alkyl-N(R³)—C(S)—C₀-C₃alkyl-heteroaryl, R³-heterocyclyl-C₀-C₃alkyl, R³-cycloalkyl-C₀-C₃alkyl, (R³)(R^{3a})N—C₂-C₄alkyl-O-aryl-, (R³)(R^{3a})N—C₂-C₄alkyl-S(O)₀₋₂-aryl-, (R³)(R^{3a})N—C₂-C₄alkyl-O-heteroaryl-, (R³)(R^{3a})N—C₂-C₄alkyl-S(O)₀₋₂-heteroaryl-, C₀-C₃alkyl-aryl-C₀-C₃alkyl, CO—C₃alkyl-heteroaryl-C₀-C₃alkyl, aryl-C₁-C₃alkyl-heteroaryl, aryl-C₁-C₃alkyl-aryl, heteroaryl-C₁-C₃alkyl-aryl, heteroaryl-C₁-C₃alkyl-heteroaryl, R³-heterocyclyl-C₀-C₃alkyl-N(R³)—C(O)—N(R³)-heteroaryl-, R³-heterocyclyl-C₁-C₃alkyl-N(R³)—C(O)—N(R³)-aryl-, R³-cycloalkyl-C₀-C₃alkyl-N(R³)—C(O)—N(R³)-heteroaryl-, R³-cycloalkyl-C₁-C₃alkyl-N(R³)—C(O)—N(R³)-aryl-, R³-heterocyclyl-C₀-C₃alkyl-N(R³)—C(S)—N(R³)-heteroaryl-, R³-heterocyclyl-C₁-C₃alkyl-N(R³)—C(S)—N(R³)-aryl-, R³-cycloalkyl-C₀-C₃alkyl-N(R³)—C(S)—N(R³)-heteroaryl-, R³-cycloalkyl-C₁-C₃alkyl-N(R³)—C(S)—N(R³)-aryl-, heteroaryl-C(O)—C₀-C₆alkyl-heteroaryl-, heteroaryl-C(O)—C₀-C₆alkyl-aryl-, aryl-C(O)—C₀-C₆alkyl-aryl-, aryl-C(O)—C₀-C₆alkyl-heteroaryl-, heteroaryl-C(O)—N(R³)—C₀-C₆alkyl-aryl-, heteroaryl-C(O)—N(R³)—C₀-C₆alkyl-heteroaryl-, heteroaryl-S(O)₂—C₀-C₆alkyl-heteroaryl-, heteroaryl-S(O)₂—CO—C₆alkyl-aryl-, aryl-S(O)₀₋₂—C₀-C₆alkyl-aryl, aryl-C₀-C₃alkyl-S(O)₀₋₂—C₀-C₆alkyl-heteroaryl, R³—O—C(O)—N(R³)—C₀-C₃alkyl-heteroaryl-, R³—O—C(O)—N(R³)—C₀-C₃alkyl-aryl-, R³—O—C(S)—N(R³)—C₀-C₃alkyl-heteroaryl-, R³—O—C(S)—N(R³)—C₀-C₃alkyl-aryl-, R³—C(O)-heterocyclyl-C₀-C₃alkyl-heteroaryl-R³—C(O)-heterocyclyl-C₀-C₃alkyl-aryl-, R³—C(O)-cycloalkyl-C₀-C₃alkyl-heteroaryl-, R³—C(O)-cycloalkyl-C₀-C₃alkyl-aryl-, R³-heterocyclyl-C₀-C₃alkyl-N(R³)—C(O)—N(R³)—C₀-C₃alkyl-heteroaryl-, R³-heterocyclyl-C₀-C₃alkyl-N(R³)—S(O)₂—C₀-C₃alkyl-heteroaryl-, R³-heterocyclyl-C₀-C₃alkyl-N(R³)—S(O)₂—C₀-C₃alkyl-aryl-, R³-cycloalkyl-C₀-C₃alkyl-N(R³)—S(O)₂—C₀-C₃alkyl-heteroaryl-, R³-cycloalkyl-C₀-C₃alkyl-N(R³)— S(O)₂—C₀-C₃alkyl-aryl-, R³-heterocyclyl-C₀-C₃alkyl-N(R³)—C(O)—C₀-C₃alkyl-heteroaryl-, R³-heterocyclyl-C₀-C₃alkyl-N(R³)—C(O)—C₀-C₃alkyl-aryl-, R³-cycloalkyl-C₀-C₃alkyl-N(R³)—C(O)—C₀-C₃alkyl-heteroaryl-, R³-cycloalkyl-C₀-C₃alkyl-N(R³)—C(O)—C₀-C₃alkyl-aryl-, R³-heterocyclyl-C₀-C₃alkyl-N(R³)—C(S)—C₀-C₃alkyl-heteroaryl-, R³-heterocyclyl-C₀-C₃alkyl-N(R³)—C(S)—C₀-C₃alkyl-aryl-, R³-cycloalkyl-C₀-C₃alkyl-N(R³)—C(S)—C₀-C₃alkyl-heteroaryl-, R³-cycloalkyl-C₀-C₃alkyl-N(R³)—C(S)—C₀-C₃alkyl-aryl-, R³—C(O)—C₀-C₃alkyl-heteroaryl-, R³—C(O)—C₀-C₃alkyl-aryl-, heterocyclyl-C(O)—, an aromatic polycycle, a non-aromatic polycycle, a mixed aryl and non-aryl polycycle, a polyheteroaryl, a non-aromatic polyheterocycle, and a mixed aryl and non-aryl polyheterocycle, each of which is optionally substituted with one or more groups selected from R³, R⁴ or R⁷;
or
Y is selected from the group consisting of
A^{2a}-aryl-C₀-C₃alkyl-N(R³)—C(O)—C₁-C₇alkyl-, wherein the C₁-C₇alkyl is optionally substituted with a moiety selected from the group consisting of —N(R³)—C(O)—C₁-C₅alkyl-(C₂-C₄alkenyl)₀₋₁-C₁-C₃alkyl-O-A^{2b}, —N(R³)—C(O)—N(R³)—C₁-C₅alkyl-(C₂-C₄ alkenyl)₀₋₁-C₁-C₃alkyl-O-A^{2b}, —N(R³)—C(O)—O—C₁-C₅alkyl-(C₂-C₄alkenyl)₀₋₁-C₁-C₃alkyl-O-A^{2b}, —N(R³)—C₁-C₅alkyl-(C₂-C₄alkenyl)₀₋₁-C₁-C₃alkyl-O-A^{2b}, —N(R³)—S(O)₂—N(R³)—C₁-C₅alkyl-(C₂-C₄alkenyl)o—C₁-C₃alkyl-O-A^{2b} and —N(R³)—S(O)₂—C₁-C₅alkyl-(C₂-C₄alkenyl)₀₋₁-C₁-C₃alkyl-O-A^{1b}
A^{2a}-heteroarylene-C₀-C₃alkyl-N(R³)—C(O)—C₁-C₇alkyl-, wherein the C₁-C₇alkyl is optionally substituted with a moeity selected from the group consisting of —N(R³)—C(O)—C₁-C₅alkyl-(C₂-C₄alkenyl)₀₋₁-C₁-C₃alkyl-O-A^{2b}, —N(R³)—C(O)—N(R³)—C₁-C₅alkyl-(C₂-C₄alkenyl)₀₋₁-C₁-C₃alkyl-O-A^{2b}, —N(R³)—C(O)—O—C₁-C₅alkyl-(C₂-C₄alkenyl)₀₋₁-C₁-C₃alkyl-O-A^{2b}, —N(R³)—C₁-C₅alkyl-(C₂-C₄alkenyl)₀₋₁-C₁-C₃alkyl-O-A^{2b}, —N(R³)—S(O)₂—N(R³)—C₁-C₅alkyl-(C₂-C₄alkenyl)₀₋₁-C₁-C₃alkyl-O-A^{2b} and —N(R³)—S(O)₂—C₁-C₅alkyl-(C₂-C₄alkenyl)o—C₁-C₃alkyl-O-A^{2b} and
B²—B¹—N(R³)—C(O)—C₁-C₇alkyl-, wherein the C₁-C₇alkyl is optionally substituted with —NR³—B³ wherein the amine of B³ is connected with the acid of B² to form a peptide bond; and wherein
A^{2a} and A^{2b} together are a covalent bond and are attached to form a ring; and
B¹, B² and B³ are each independently a natural or synthetic amino acid and when any of B¹, B² and B³ are linked together they are linked together via a peptide bond;
each R³ and R^{3a} are independently selected from the group consisting of —H, —OH, —C(O)H, heterocyclyl, C₁-C₆alkyl, C₂-C₆alkenyl, C₂-C₆alkynyl, —C₂-C₄alkyl-OR^{a}—C(O)—O—C₂-C₄alkyl-NR^{a}R^{a}, heteroalkyl, C₀-C₆alkylheteroaryl, C(O)CF₃, —C(O)—NH₂, —C(O)—NH—C₁-C₆alkyl, —NH₂, C₃-C₆cycloalkyl, —C₁-C₆alkylaryl, heteroaryl-aryl, aryl and alkylheteroaryl, wherein each alkyl, alkenyl, alkynyl, heteroalkyl, cycloalkyl, heterocyclyl, aryl and heteroaryl moiety is optionally substituted;
each R⁴ is independently selected from the group consisting of —H, —C(NR^{a})—N(R^{a})₂, C₁-C₆alkyl, C₂-C₆alkenyl, C₂-C₆alkynyl, —C₁-C₆alkyl-R^{a}, —C₀-C₆alkyl-O—R^{a}, —C₀-C₆alkyl-S(O)₀₋₂—R^{a}, —C₀-C₆alkyl-C(O)—OR^{a}, —C₀-C₆alkyl-C(O)—N(R³)(R^{3a}), —C₀-C₆alkyl-C(S)—N(R³)(R^{3a}), —CH═CH—C(O)—OR^{a}, —CH═CH—C(O)—N(R³)(R^{3a}), —CH═CH—C(S)—N(R³)(R^{3a}), —N(R³)—C(O)—CF₃, —N(R³)—C₂-C₆alkyl-N(R³)(R^{3a}), —C₀-C₆alkyl-N(R³)(R^{3a}), —N(R³)—C(O)—C₁-C₆alkyl-R³, —N(R³)—C(S)—C₁-C₆alkyl-R³, —N(R³)—S(O)₂—C₁-C₆alkyl-R³, —S(O)₂—N(R³)R^{3a}, —O—C₂-C₆alkyl-N(R³)(R^{3a}), —S(O)₀₋₂—C₂-C₆alkyl-N(R³)(R^{3a}), —S—R³, —S(O)₀₋₂—C₂-C₆alkyl-R³, —C₃-C₆cycloalkyl, —C₃-C₆cycloalkyl-R^{a}, heterocyclyl, —C₄-C₇heterocyclyl-R^{a}, —O—C₀-C₄alkyl-cycloalkyl, —S(O)₀₋₂—C₀-C₄alkyl-cycloalkyl, —O—C₂-C₄alkyl-heterocyclyl (when the alkyl is linked via a N in the heterocyclyl), —O—C₀-C₄alkyl-heterocyclyl (when the alkyl is linked via a C in the heterocyclyl), —S(O)₀₋₁—C₀-C₄alkyl-heterocyclyl (when the alkyl is linked via a C in the heterocyclyl), —S(O)₀₋₁—, —C₂-C₄alkyl-heterocyclyl (when the alkyl is linked via a N in the heterocyclyl, —S(O)₂—C₀-C₄alkyl-heterocyclyl, —O—C₀-C₄alkyl-heterocyclyl-C(O)—OR^{a} (when the alkyl is linked via a C in the heterocyclyl, —O—C₂-C₄alkyl-heterocyclyl-C(O)—OR^{a} (when the alkyl is linked via a N in the heterocyclyl), —O-cycloalkyl-C(O)—OR^{a}, —O—C(O)OR^{a}, —O-heteroaryl-C(O)—OR^{a}, —S(O)₀₋₁—C₀-C₄alkyl-heterocyclyl-C(O)—OR^{a} (when the alkyl is linked via a C in the heterocyclyl), —S(O)₀₋₁—C₂-C₄alkyl-heterocyclyl-C(O)—OR^{a} (when the alkyl is linked via a N in the heterocyclyl), —S(O)₂—C₀-C₄alkyl-heterocyclyl-C(O)—OR^{a}, —S(O)₀₋₂-cycloalkyl-C(O)—OR^{a}, —S(O)₀₋₂-aryl-C(O)—OR^{a}, —S(O)₀₋₂-heteroaryl-C(O)—OR^{a}, —O—C₀-C₄alkyl-aryl, —S(O)₀₋₂—C₀-C₄alkyl-aryl, —O—C₀-C₄alkyl-heteroaryl, —S(O)₀₋₂—C₀-C₄alkyl-heteroaryl, —O—C(O)—N(R³)—C₀-C₄alkyl-aryl, —O—C(S)—N(R³)—C₀-C₄alkyl-aryl, —O—C(O)—N(R³)—C₀-C₄alkyl-heteroaryl, —O—C(S)—N(R³)—C₀-C₄alkyl-heteroaryl, —O—C(O)—N(R³)—C₀-C₄alkyl-cycloalkyl, —O—C(S)—N(R³)—C₀-C₄alkyl-cycloalkyl, —O—C(O)—N(R³)—C₀-C₄alkyl-heterocyclyl, —O—C(S)—N(R³)—C₀-C₄alkyl-heterocyclyl, —O—C₀-C₄alkyl-heterocyclyl-aryl (when the alkyl is linked via a C in the heterocyclyl), —O—C₂-C₄alkyl-heterocyclyl-aryl (when the alkyl is linked via a N in the heterocyclyl), —O—C₀-C₄alkyl-heterocyclyl-heteroaryl (when the alkyl is linked via a C in the heterocyclyl), —O—C₂-C₄alkyl-heterocyclyl-heteroaryl (when the alkyl is linked via a N in the heterocyclyl), —O—C₀-C₄alkyl-heterocyclyl-cycloalkyl (when the alkyl is linked via a C in the heterocyclyl), —O—C₂-C₄alkyl-heterocyclyl-cycloalkyl (when the alkyl is linked via a N in the heterocyclyl), —O—C₀-C₄alkyl-heterocyclyl-heterocyclyl (when the alkyl is linked via a C in the heterocyclyl), —O—C₂-C₄alkyl-heterocyclyl-heterocyclyl (when the alkyl is linked via a N in the heterocyclyl), —S(O)₀₋₁—C₀-C₄alkyl-heterocyclyl-aryl (when the alkyl is linked via a C in the heterocyclyl), —S(O)₀₋₁—C₂-C₄alkyl-heterocyclyl-aryl (when the alkyl is linked via a N in the heterocyclyl), —S(O)₂—C₀-C₄alkyl-heterocyclyl-aryl, —S(O)₀₋₁—C₀-C₄alkyl-heterocyclyl-heteroaryl (when the alkyl is linked via a C in the heterocyclyl), —S(O)₀₋₁—C₂-C₄alkyl-heterocyclyl-heteroaryl (when the alkyl is linked via a N in the heterocyclyl), —S(O)₂—C₀-C₄alkyl-heterocyclyl-heteroaryl, —S(O)₀₋₁—C₀-C₄alkyl-heterocyclyl-cycloalkyl (when the alkyl is linked via a C in the heterocyclyl), —S(O)₀₋₁—C₂-C₄alkyl-heterocyclyl-cycloalkyl (when the alkyl is linked via a N in the heterocyclyl), —S(O)₂—C₀-C₄alkyl-heterocyclyl-cycloalkyl, —S(O)₀₋₁—C₀-C₄alkyl-heterocyclyl-heterocyclyl (when the alkyl is linked via a C in the heterocyclyl), —S(O)₀₋₁—C₂-C₄alkyl-heterocyclyl-heterocyclyl (when the alkyl is linked via a N in the heterocyclyl), —S(O)₂—C₀-C₄alkyl-heterocyclyl-heterocyclyl, —N(R³)—C₂-C₄alkyl-heterocyclyl, —N(R³)—C₂-C₄alkyl-cycloalkyl, —N(R³)—C₂-C₄alkyl-heteroaryl, —N(R³)—C₂-C₄alkyl-aryl, —N(R³)—C(O)—N(R³)—C₀-C₄alkyl-heterocyclyl-R³, —N(R³)—C(S)—N(R³)—C₀-C₄alkyl-heterocyclyl-R³, —N(R³)—C(O)—N(R³)—C₀-C₄alkyl-cycloalkyl-R³, —N(R³)—C(S)—N(R³)—C₁-C₄alkyl-cycloalkyl-R³, —N(R³)—C(O)—N(R³)—C₀-C₄alkyl-aryl-R³, —N(R³)—C(S)—N(R³)—C₀-C₄alkyl-aryl-R³, —N(R³)—C(O)—N(R³)—C₀-C₄alkyl-heteroaryl-R³, —N(R³)—C(S)—N(R³)—C₀-C₄alkyl-heteroaryl-R³, —C₀-C₄alkyl-O—C(O)—R^{a}, —C₀-C₄alkyl-N(R³)—C(O)—O—R^{a}, —C₀-C₄alkyl-N(R³)—C(S)—O—R^{a}, —C₀-C₄alkyl-heterocyclyl-C(O)—O—R^{a}, —C₀-C₄alkyl-cycloalkyl-C(O)—O—R^{a}, —C₀-C₄alkyl-hetero aryl-C(O)—O—R^{a}, —C₀-C₄alkyl-aryl-(O)—R^{a}—N(R³)—C₂-C₄alkyl-heterocyclyl, —N(R³)—C₂-C₄alkyl-cycloalkyl, —N(R³)—C₂-C₄alkyl-heteroaryl, —N(R³)—C₂-C₄alkyl-aryl, F, Cl, Br, I, —CF₃, —SO₃H, —CN, aryl, heteroaryl, cycloalkyl and heterocyclyl, wherein each alkyl, alkenyl, alkynyl, cycloalkyl, heterocyclyl, aryl and heteroaryl moeity of the aforementioned R⁴ is optionally substituted;
each R⁷ and R^{7a} is independently selected from the group consisting of —H, C₁-C₆alkyl-, C₂-C₆alkenyl, C₂-C₆alkynyl, C₁-C₆heteroalkyl, R^{a}—O—C₂-C₆alkyl-, R^{a}—S(O)₀₋₂—C₂-C₆alkyl-, N(R³)(R^{3a})—C₂-C₆alkyl-, a protecting group, C₁-C₆alkyl-O—C(O)—, aryl-C₀-C₄alkyl-O—C(O)—, heteroaryl-C₀-C₄alkyl-O—C(O)—, benzyl-O—C(O)—, heterocyclyl-C₁-C₆alkyl-, cycloalkyl-C₁-C₆alkyl-, heteroaryl-C₁-C₆alkyl-, aryl-C₁-C₆alkyl-, wherein each alkyl, alkenyl, alkynyl, heteroalkyl, aryl, heteroaryl, cycloalkyl, benzyl and heterocyclyl moiety is independently optionally substituted; provided that
R⁷ is —OR^{a} when attached to the N atom of an indolyl moiety; and
wherein in a —N(R³)(R^{3a}) group, optionally the R³ and R^{3a} together with the nitrogen atom to which they are attached form a heterocyclyl group.
with the proviso that Formula (I) excludes compounds of Formula (X-1), Formula (X-2), Formula (X-3) and Formula (X-4): 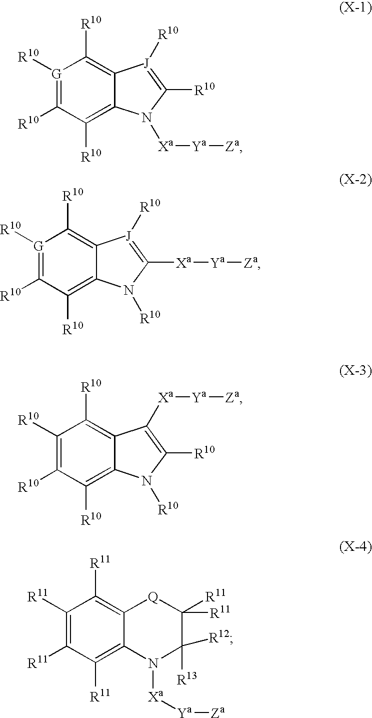
wherein,
G is N or C, subject to the proviso that R¹⁰ is absent when G is N;
J is N or C, subject to the proviso that R¹⁰ is absent when J is N;
Q is selected from the group consisting of S, O, SO₂ and NR¹¹;
X^{a} is —C(O)—, —S(O)₂— or a covalent bond;
Y^{a} is selected from the group consisting of alkyl, alkenyl, cycloalkyl, alkylcycloalkyl, alkylcycloalkylalkyl, alkyloxyalkyl, aryl, alkyaryl, alkylarylalkyl, arylalkyl, cycloalkylalkyl, alkylheterocycle, heterocyclealkyl, alkylheterocyclealkyl, heterocycle, aminoalkyl, oxyalkyl, aminoaryl and oxyaryl when 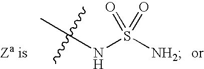
Y^{a} is selected from the group consisting of aminoalkyl and aminoaryl when
Z^{a} is selected from the group consisting of 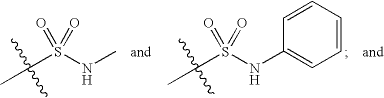
each of R¹⁰, R¹¹, R¹² and R¹³ is independently selected from the group consisting of R⁴; or
R¹² and R¹³ together are ═O or ═S; and
with the proviso that Formula (I) excludes compounds of Formula (X-5): 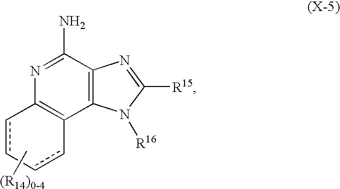
wherein each R¹⁴ is independently selected from the group consisting of C₁₋₁₀alkyl, C₁₋₁₀alkoxy, halogen and trifluoromethyl;
R¹⁵ is selected from the group consisting of R⁴;
R¹⁶ is -alkyl-N(R¹⁷)—S(O)₂—N(R¹⁹)—R¹⁸ or -alkenyl-N(R¹⁷)—S(O)₂—N(R¹⁹)—R¹⁸;
R¹⁷ is hydrogen or C₁₋₁₀alkyl;
R¹⁸ is selected from the group consisting of aryl, heteroaryl, heterocyclyl, alkyl or alkenyl, each of which is optionally substituted; and
R¹⁹ is selected from the group consisting of H and C₁₋₁₀alkyl, or R¹⁸ and R¹⁹ can combine to form a 3 to 7 membered heterocyclic or substituted heterocyclyl ring; and
with the proviso that Formula (I) excludes compounds of Formula (X-6): 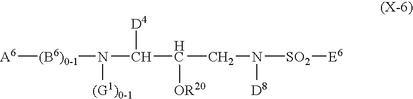
wherein
A⁶ is selected from the group consisting of C₃₋₇cycloalkyl, C₅₋₇cycloakenyl, C₆₋₁₀aryl and a 5-7 membered saturated or unsaturated heterocycle, each optionally substituted;
B⁶ is selected from the group consisting of —N(R²¹)—C(R²²)₂—C(O)—
R²¹ is selected from H and C₁₋₄alkyl optionally substituted with a 3-7 membered saturated or unsaturated carbocyclic ring system or a 5-7 membered saturated or unsaturated heterocyclic ring;
each R²² is independently selected from the group consisting of H, C₃₋₇cycloalkyl, C₅₋₇cycloalkenyl, C₆₋₁₀aryl, 5-7 membered saturated or unsaturated heterocycle, C₁₋₆alkyl, C₂₋₆alkenyl, each of which except H is optionally substituted;
G¹ is selected from H and C₁₋₄alkyl;
D⁴ and D⁸ are independently selected from a 3-7 membered saturated or unsaturated optionally substituted carbocyclic ring system, a 5-7 membered saturated or unsaturated optionally substituted heterocyclic ring, C₁₋₆alkyl, which is optionally substituted with one or more groups selected from C₃₋₆cycloalkyl, —OR²¹, R²², —O-(3-7 membered saturated or unsaturated optionally substituted carbocyclic ring system), —O-(5-7 membered saturated or unsaturated optionally substituted heterocyclic ring), 3-7 membered saturated or unsaturated optionally substituted carbocyclic ring system, and 5-7 membered saturated or unsaturated optionally substituted heterocyclic ring; C₂₋₄alkenyl, which is substituted with one or more groups selected from C₃₋₆cycloalkyl, —OR²¹, —R²²—O-(3-7 membered saturated or unsaturated optionally substituted carbocyclic ring system), —O-(5-7 membered saturated or unsaturated optionally substituted heterocyclic ring), 3-7 membered saturated or unsaturated optionally substituted carbocyclic ring system, and 5-7 membered saturated or unsaturated optionally substituted heterocyclic ring; C₃₋₆cycloalkyl, which is optionally substituted with or fused to a 5-7 membered saturated or unsaturated optionally substituted heterocyclic ring or a 3-7 membered saturated or unsaturated optionally substituted carbocyclic ring system; and C₅₋₆cycloalkenyl, optionally substituted with or fused to a 3-7 membered saturated or unsaturated optionally substituted carbocyclic ring system, or a 5-7 membered saturated or unsaturated optionally substituted heterocyclic ring; 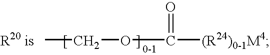
R²⁴ is C(R²¹)₂, O or N(R²¹);
M⁴ is selected from the group consisting of H, optionally substituted C₁₋₁₂alkyl, optionally substituted C₂₋₁₂alkenyl, a 5-6 membered saturated or unsaturated optionally substituted carbocyclic or heterocyclic ring, an 8-10 membered saturated or unsaturated optionally substituted bicyclic ring system, wherein 1 to 4-CH₂ radicals of the alkyl or alkenyl group is optionally replaced by a heteroatom group selected from O, S, S(O), S(O)₂ and N(R²¹); and
E⁶ is selected from the group consisting of —O—R²² and —N(R²¹)(R²²); and
with the proviso that Formula (I) excludes compounds of Formula (X-7): 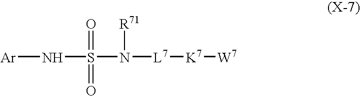
wherein,
Ar is selected from the group consisting of a mono-cyclic aryl, a mono-cyclic heteroaryl, a bicyclic aryl and a bicyclic heteroaryl
R⁷¹ is selected from the group consisting of H and C₁₋₃ straight chained alkyl, wherein the alkyl is optionally substituted with a group selected from H, OR⁷⁵, CN, C₁₋₆alkyl, CH₂OR⁷⁵, CON(R⁷⁵)₂, CO₂R⁷⁵, phenyl, pyridyl, thiophenyl, and naphthyl;
each R⁷⁵ is independently selected from the group consisting of H, C₁₋₃alkyl, C₁₋₃monohaloalkyl and C₁₋₃polyhaloalkyl;
L⁷ is selected from the group consisting of —C₃₋₉alkyl-, —C₃₋₉alkenyl-, —C₃₋₉alkynyl-, 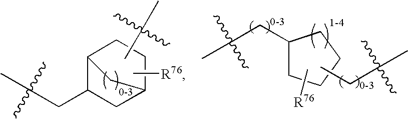
and optionally substituted 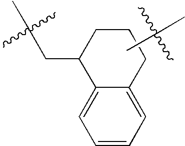
wherein one dashed line is a double bond and the other dashed line is a single bond;
each R⁷⁶ is independently selected from the group consisting of H, CN, OR⁷⁵, C₁₋₅alkyl, CH₂OR⁷⁵, CON(R⁷⁵)₂, CO₂R⁷⁵, phenyl, pyridyl, thiophenyl and naphthyl, wherein the phenyl, pyridyl, thiophenyl and naphthyl is substituted with H, F, Cl, Br, I, CF₃, C₁₋₄alkyl, C₁₋₄alkoxy, C₁₋₄alkylthio or NO₂,
K⁷ is selected from the group consisting of —CH₂—NR⁷¹⁰—CHR⁷⁷—(CH₂)₀₋₃—, —CH₂—NR⁷¹⁰—CO—(CH₂)₀₋₃—, —CH₂—NH—CO—NH—(CH₂)₀₋₃—, —CO—NH—CHR⁷⁷—(CH₂)₀₋₃—, —CH₂—NR⁷¹⁰—CO—CHR⁷⁷—(CH₂)₀₋₃—, —CH₂—NR⁷¹⁰—CS—(CH₂)₀₋₃—, —CH₂—NH—CS—NH—(CH₂)₁₋₄, —CS—NH—CHR⁷⁷—(CH₂)₀₋₃—, —CH₂—NR⁷¹⁰—CS—CHR⁷⁷—(CH₂)₀₋₃— and —CH₂—N═CSR⁷¹—NH—(CH₂)₀₋₃—;
R⁷⁷ is selected from the group consisting of H, C₁₋₆alkyl, CH₂OR⁷⁵, —(CH₂)₀₋₂NHCO₂R⁷⁵, —(CH₂)₀₋₂NHSO₂R⁷⁵, CH₂N(R⁷¹¹)₂, phenyl, pyridyl, thiophenyl and naphthyl,
W⁷ is selected from the group consisting of a mono-cyclic aryl, a mono-cyclic heteroaryl, a bicyclic aryl, a bicyclic heteroaryl, a fused aryl-cycloalkyl, and a fused aryl-heterocyclyl, each of which is optionally substituted;
R⁷¹⁰ is H or C₁₋₆alkyl; and
R⁷¹¹ is selected from the group consisting of H, COR⁷⁵, COR^{a}SO₂R⁷⁵ and SO₂R⁷¹²; wherein
R⁷¹² is selected from the group consisting of phenoxy, phenyl, pyridyl, thiophenyl, and naphthyl, wherein the phenoxy, phenyl, pyridyl, thiophenyl and naphthyl is substituted with H, F, Cl, Br, I, CF₃, C₁₋₄alkyl, C₁₋₄alkoxy, C₁₋₄alkylthio, NO₂, phenyl, pyridyl, and thiophenyl;
with the proviso that Formula (I) excludes the compounds: 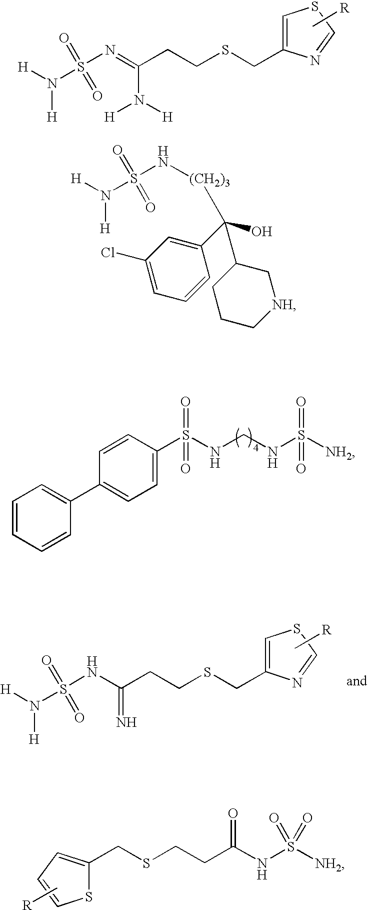
wherein R is a substituent;
with the proviso that Formula (I) excludes compounds of Formula (X-8) and Formula (X-9): 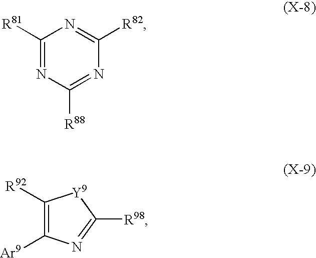
wherein
R⁸¹ is selected from the group consisting of F, Cl, Br, I, NR⁸³R⁸⁴, phenyl and heteroaryl, wherein the phenyl and heteroaryl may be substituted with one or more of F, Cl, Br, I, —CN, —NO₂, —NR⁸⁵R⁸⁶, —SO₂R⁸⁵, —(CH₂)₀₋₆C(Y⁸)R⁸⁷, —(CH₂)₀₋₆—Y⁸R⁸⁵, —(CH₂)₀₋₆C(Y⁸)NR⁸⁵R⁸⁶, —(CH₂)₀₋₆NR⁸⁵C(Y⁸)R⁸⁵, —(CH₂)₀₋₆CO₂R⁸⁵, —(CH₂)₀₋₆SO₂NR⁸⁵R⁸⁶, a straight chained or branched C₁₋₇alkyl, monofluoroalkyl, polyfluoroalkyl, aminoalkyl, C₂₋₇alkenyl, C₂₋₇alkynyl, C₃₋₇cycloalkyl and C₃₋₇cycloalkenyl;
R⁸² is NR⁸³R⁸⁴;
R⁸³ is selected from the group consisting of H, —(CH₂)₂₋₄Y⁸R⁸⁵, —(CH₂)₁₋₄C(Y⁸)NR⁸⁵R⁸⁶, —(CH₂)₂₋₄NR⁸⁵C(Y⁸)R⁸⁵, —(CH₂)₁₋₄C(Y⁸)R⁸⁷, —(CH₂)₁₋₄CO₂R⁸⁵, —(CH₂)₂₋₄NR⁸⁵R⁸⁶, —(CH₂)₂₋₄CN, —C(Y⁸)R⁸⁵, —C(Y⁸)NR⁸⁵R⁸⁶, —CO₂R⁸⁵, straight chained or branched C₁₋₇alkyl, C₂₋₇alkenyl, C₂₋₇alkynyl, C₃₋₇cycloalkyl, C₃₋₇cycloalkenyl, phenyl, C₁₋₆phenylalkyl and C₁₋₆heteroarylalkyl, wherein the phenyl, C₁₋₆phenylalkyl and C₁₋₆heteroarylalkyl may be substituted with one or more of F, Cl, Br, I, —CN, —NO₂, —NR⁸⁵R⁸⁶, —SO₂R⁸⁵, —(CH₂)₀₋₆C(Y⁸)R⁸⁷, —(CH₂)₀₋₆—Y⁸R⁸⁵, —(CH₂)₀₋₆C(Y⁸)NR⁸⁵R⁸⁶, —(CH₂)₀₋₆NR⁸⁵C(Y⁸)R⁸⁵, —(CH₂)₀₋₆CO₂R⁸⁵—(CH₂)₀₋₆SO₂NR⁸⁵R⁸⁶, a straight chained or branched C₁₋₇alkyl, monofluoroalkyl, polyfluoroalkyl, aminoalkyl, C₂₋₇alkenyl, C₂₋₇alkynyl, C₃₋₇cycloalkyl and C₃₋₇cycloalkenyl;
R⁸⁴ is selected from the group consisting of H, —(CH₂)₂₋₄Y⁸R⁸⁵, —(CH₂)₁₋₄C(Y⁸)NR⁸⁵R⁸⁶, —(CH₂)₂₋₄NR⁸⁵C(Y⁸)R⁸⁵, —(CH₂)₁₋₄C(Y⁸)R⁸⁷, —(CH₂)₁₋₄CO₂R⁸⁵, —(CH₂)₂₋₄NR⁸⁵R⁸⁶, —(CH₂)₂₋₄CN, straight chained or branched C₁₋₇alkyl, C₂₋₇alkenyl, C₂₋₇alkynyl, C₃₋₇cycloalkyl, C₃₋₇cycloalkenyl, phenyl, C₁₋₆phenylalkyl, wherein the phenyl and C₁₋₆phenylalkyl may be substituted with one or more of F, Cl, Br, I, —CN, —NO₂, —NR⁸⁵R⁸⁶, —SO₂R⁸⁵, —(CH₂)₀₋₆C(Y⁸)R⁸⁷, —(CH₂)₀₋₆—Y₈R⁸⁵, —(CH₂)₀₋₆C(Y⁸)NR⁸⁵R⁸⁶, —(CH₂)₀₋₆NR⁸⁵C(Y⁸)R⁸⁵, —(CH₂)₀₋₆CO₂R⁸⁵, —(CH₂)₀₋₆SO₂NR⁸⁵R⁸⁶, a straight chained or branched C₁₋₇alkyl, monofluoroalkyl, polyfluoroalkyl, aminoalkyl, C₂₋₇alkenyl, C₂₋₇alkynyl, C₃₋₇cycloalkyl and C₃₋₇cycloalkenyl; or
R⁸³ and R⁸⁴ taken together with the nitrogen atom to which they are attached are 1-azetidinyl, 1-pyrrolidinyl, 1-piperidinyl or 1H-azepanyl, each of which is optionally substituted;
or R⁸³ and R⁸⁴ taken together with the nitrogen atom to which they are attached are morpholinyl, thiomorpholinyl, [1,4]oxazepanyl, [1,4]thiazepanyl, piperazinyl or [1,4]diazepanyl, each of which is optionally substituted;
each of R⁸⁵, R⁸⁶ and R⁸⁷ is independently selected from the group consisting of H and straight chained or branched C₁₋₇alkyl;
Y⁸ is O or S;
Y⁹ is selected from the group consisting of O, S and NH;
R⁸⁸ is selected from the group consisting of 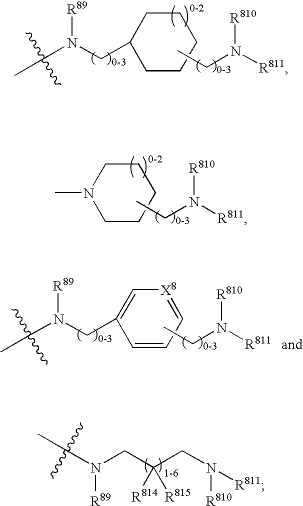
R⁹⁸ is selected from the group consisting of 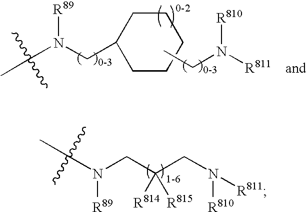
each of R⁸⁹ and R⁸¹⁰ is independently H or a straight chained or branched C₁₋₄alkyl;
R⁸¹¹ is —SO₂—NR⁸³R⁸⁴;
R⁸¹⁴ is selected from the group consisting of H, straight chained or branched C₁₋₇alkyl, F and —(CH₂)₀₋₆OR⁸⁵;
—R⁸¹⁵ is selected from the group consisting of H, straight chained or branched C₁₋₇alkyl and F;
Ar⁹ is a heteroaryl ring;
R⁹² is selected from the group consisting of H, straight chained or branched C₁₋₄alkyl, —(CH₂)₁₋₄OR⁹⁵, phenyl optionally substituted with one or more of F, Cl, Br, CF₃, CN, NO₂, NR⁹⁵R⁹⁶, —SO₂R⁹⁵, —(CH₂)₀₋₆OR⁹⁵ and straight chained or branched C₁₋₄alkyl;
X⁸ is N or C; and
R⁹⁵ and R⁹⁶ are independently H or straight chained or branched C₁₋₇alkyl; and
with the proviso that Formula (I) excludes those compounds wherein when: 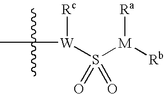
is NR^{c}—SO₂—NR^{a}R^{b}, wherein
each of R^{a}, R^{b} and R^{c} is independently selected from the group consisting of H and C₁₋₄alkyl; then Y-L-Z- is not 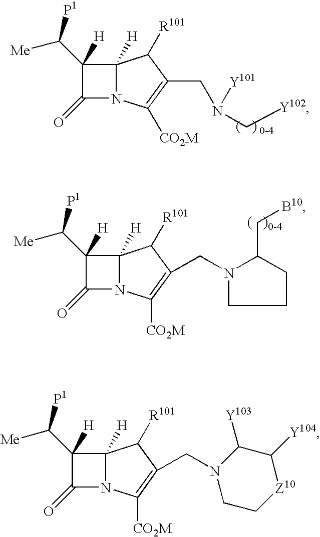
wherein
P¹ is H or hydroxy;
R¹⁰¹ is H or methyl;
Y¹⁰¹ is H or C₁₋₄alkyl;
the group CO₂M is selected from the group consisting of a carboxylic acid, a carboxylate anion, a pharmaceutically acceptable ester group, and a carboxylic acid protected by a protecting group;
Y¹⁰² is —C(O)— or absent and Y¹⁰² is the point of attachment to W of Formula (I);
Z¹⁰ is —O—, —S—, —N(H)— or —N(C₁-C₄alkyl)-;
B¹⁰ is —C(O)— or absent and B¹⁰ is the point of attachment to W of Formula (I); and
one of Y¹⁰³ and Y¹⁰⁴ is selected from the group consisting of H and —(CH₂)₀₋₄-A¹⁰, wherein A¹⁰ is selected from the group consisting of —N(R¹⁰²)₂, —CO₂R¹⁰², —CO—N(R¹⁰²)₂, —CO—NR¹⁰²SO₂N(R¹⁰²)₂, —NR¹⁰²SO₂N(R¹⁰²)₂, —NH—C(═NR¹⁰²)—N(R¹⁰²)₂, and —S—C(═NR¹⁰²)—N(R¹⁰²)₂, and the other one of Y¹⁰³ and Y¹⁰⁴ is the point of attachment to W of Formula (I) and is either —C(O)— or absent, wherein each R¹⁰² is independently H or C₁₋₄alkyl; and
with the proviso that Formula (I) excludes those compounds wherein Y-L-Z- is selected from the group consisting of aryl-(CH₂)₂—, heteroaryl-(CH₂)₂—, heterocycle-(CH₂)₂— and cycloalkyl-(CH₂)₂—.

2. The compound according to claim 1, wherein each alkyl, alkenyl, alkynyl, heteroalkyl, cycloalkyl, heterocyclyl, aryl and heteroaryl moiety of Y, L, Z, R^{a}, R^{b}, R^{c}, R³ and R^{3a} is independently optionally substituted with one or more groups independently selected from R⁴.

3. The compound according to claim 2, wherein each alkyl, alkenyl, alkynyl, heteroalkyl, cycloalkyl, heterocyclyl, aryl and heteroaryl moiety of Y, L, Z, R^{a}, R^{b}, R^{c}, R³ and R^{3a} is independently optionally substituted with one or more groups independently selected from oxo, —OH, —CN, C₁-C₆alkyl, C₁-C₆alkoxy, —NO₂, —N(R^{a})₂, —N(R⁷)(R^{7a}), R⁴, halo, —SH, —S—C₁-C₆alkyl, —S(O)—C₁-C₆alkyl, —S—C(O)—C₁-C₆alkyl and mono- to per-halogenated C₁-C₆alkyl.

4. The compound according to claim 1, wherein a C₁-C₆alkyl moiety of an R⁴ is optionally substituted with a substituent selected from the group consisting of —OH, —NO₂ and C₀-C₆ alkyl-C(O)—N(R³)(R^{3a}).

5. The compound according to claim 1, wherein each alkyl, alkenyl, alkynyl, heteroalkyl, aryl, heteroaryl, cycloalkyl and heterocyclyl moiety of Z is independently optionally substituted with one or more substituents independently selected from the group consisting of oxo, —OH, —CN, C₁-C₆alkyl, C₁-C₆alkoxy, —NO₂, —N(R³)(R^{3a}), halo, —SH and mono- to per-halogenated C₁-C₆alkyl.

6. The compound according to claim 1, wherein L is selected from the group consisting of
—C₀-C₆alkyl-N(R³)—C₀-C₃alkyl-, wherein when the —C₀-C₆alkyl is —C₁-C₆alkyl it is optionally substituted with a substituent selected from the group consisting of —C₁-C₃alkyl-OR^{a} and —C₁-C₃alkyl-C(O)OR^{a},
—C₀-C₆alkyl-N(R³)—C(O)—C₀-C₃alkyl-, wherein when the —C₀-C₆alkyl is C₁-C₆alkyl it is optionally substituted with a substituent selected from the group consisting of —C₁-C₃alkyl-OR^{a}, —C₁-C₃alkyl-NR³R^{3a} and —C₁-C₃alkyl-C(O)OR^{a},
—C₀-C₆alkyl-N(R³)—C(O)—C₀-C₃alkyl-, wherein the —C₀-C₃alkyl is C₁-C₃alkyl it is optionally substituted with a substituent selected from the group consisting of —N(R³)—C(O)—C₀-C₃alkyl-Y, —N(R³)—C(S)—C₀-C₃alkyl-Y, —C(O)—N(R³)—C₀-C₃alkyl-Y, —C(S)—N(R³)—C₀-C₃alkyl-Y, —N(R³)—C(O)—C₀-C₃alkyl-C₄-C₆cycloalkyl-, —N(R³)—C(S)—C₀-C₃alkyl-C₄-C₆cycloalkyl-, —N(R³)—C₀-C₃alkyl-Y, —N(R³)(R^{3a}), —N(R³)—C₀-C₃alkyl-C₄-C₆heterocyclyl, —N(R³)—C₂-C₃alkyl-N(R³)(R^{3a}), —N(R³)—C₂-C₃alkyl-OR^{a}, —N(R³)—C₀-C₃alkyl-C₀-C₃heteroalkyl-Y, —N(R³)—C(O)—O—C₀-C₃alkyl-Y, —N(R³)—C(S)—O—C₀-C₃alkyl-Y, —N(R³)—S(O)₂—C₀-C₃alkyl-Y, —N(R³)—C(O)—N(R³)—C₀-C₃alkyl-Y, —N(R³)—C(S)—N(R³)—C₀-C₃alkyl-Y, —N(R³)—C(O)—C₀-C₃alkyl-C₀-C₃heterocyclyl, —N(R³)—C(S)—C₀-C₃alkyl-C₀-C₃heterocyclyl, —N(R³)—C(O)—C₀-C₃alkyl-C₀-C₃heterocyclyl-Y, —N(R³)—C(S)—C₀-C₃alkyl-C₀-C₃heterocyclyl-Y and —N(R³)—S(O)₂—N(R³)—CO—C₃alkyl-Y,
—C₀-C₆alkyl-N(R³)—C(S)—C₀-C₃alkyl-, wherein the —C₀-C₃alkyl is C₁-C₃alkyl it is optionally substituted with a substituent selected from the group consisting of —N(R³)—C(O)—C₀-C₃alkyl-Y, —N(R³)—C(S)—C₀-C₃alkyl-Y, —C(O)—N(R³)—C₀-C₃alkyl-Y, —C(S)—N(R³)—C₀-C₃alkyl-Y, —N(R³)—C(O)—C₀-C₃alkyl-C₄-C₆cycloalkyl-, —N(R³)—C(S)—C₀-C₃alkyl-C₄-C₆cycloalkyl-, —N(R³)—C₀-C₃alkyl-Y, —N(R³)(R^{3a}), —N(R³)—C₀-C₃alkyl-C₄-C₆heterocyclyl, —N(R³)—C₂-C₃alkyl-N(R³)(R^{3a}), —N(R³)—C₂-C₃alkyl-OR^{a}, —N(R³)—C₀-C₃alkyl-C₀-C₃heteroalkyl-Y, —N(R³)—C(O)—O—C₀-C₃alkyl-Y, —N(R³)—C(S)—O—C₀-C₃alkyl-Y, —N(R³)—S(O)₂—C₀-C₃alkyl-Y, —N(R³)—C(O)—N(R³)—C₀-C₃alkyl-Y, —N(R³)—C(S)—N(R³)—C₀-C₃alkyl-Y, —N(R³)—C(O)—C₀-C₃alkyl-C₀-C₃heterocyclyl, —N(R³)—C(S)—C₀-C₃alkyl-C₀-C₃heterocyclyl, —N(R³)—C(O)—C₀-C₃alkyl-C₀-C₃heterocyclyl-Y, —N(R³)—C(S)—C₀-C₃alkyl-C₀-C₃heterocyclyl-Y and —N(R³)—S(O)₂—N(R³)—CO—C₃alkyl-Y,
—C₀-C₆alkyl-C(O)—C₀-C₃alkyl-, wherein when the —C₀-C₆alkyl is C₁-C₃alkyl it is optionally substituted with a substituent selected from the group consisting of —N(R³)—C(O)—C₀-C₃alkyl-Y, —N(R³)—C(S)—C₀-C₃alkyl-Y, —C(O)—N(R³)(R^{3a}), —C(S)—N(R³)(R^{3a}), —C(O)—N(R³)—C₀-C₃alkyl-Y, —C(S)—N(R³)—C₀-C₃alkyl-Y, —N(R³)—C(O)—C₀-C₃alkyl-C₄-C₆cycloalkyl-, —N(R³)—C(S)—C₀-C₃alkyl-C₄-C₆cycloalkyl-, —N(R³)—C₀-C₃alkyl-Y, —N(R³)(R^{3a}), —N(R³)—C₀-C₃alkyl-C₄-C₆heterocyclyl, —N(R³)—C₂-C₃alkyl-N(R³)(R^{3a}), —N(R³)—C₂-C₃alkyl-OR^{a}—, —N(R³)—C₀-C₃heteroalkyl-Y, —N(R³)—C(O)—O—C₀-C₃alkyl-Y, —N(R³)—C(S)—O—C₀-C₃alkyl-Y, —N(R³)—S(O)₂—C₀-C₃alkyl-Y, —N(R³)—C(O)—N(R³)—C₀-C₃alkyl-Y, —N(R³)—C(S)—N(R³)—C₀-C₃alkyl-Y, —N(R³)—C(O)—C₀-C₃alkyl-C₀-C₃heterocyclyl, —N(R³)—C(S)—C₀-C₃alkyl-C₀-C₃heterocyclyl, —N(R³)—C(O)—C₀-C₃alkyl-C₀-C₃heterocyclyl-Y, —N(R³)—C(S)—C₀-C₃alkyl-C₀-C₃heterocyclyl-Y and —N(R³)—S(O)₂—N(R³)—C₀-C₃alkyl-Y,
—C₀-C₆alkyl-C(S)—C₀-C₃alkyl-, wherein when the —C₀-C₆alkyl is C₁-C₃alkyl it is optionally substituted with a substituent selected from the group consisting of —N(R³)—C(O)—C₀-C₃alkyl-Y, —N(R³)—C(S)—C₀-C₃alkyl-Y, —C(O)—N(R³)(R^{3a}), —C(S)—N(R³)(R^{3a}), —C(O)—N(R³)—C₀-C₃alkyl-Y, —C(S)—N(R³)—C₀-C₃alkyl-Y, —N(R³)—C(O)—C₀-C₃alkyl-C₄-C₆cycloalkyl-, —N(R³)—C(S)—C₀-C₃alkyl-C₄-C₆cycloalkyl-, —N(R³)—C₀-C₃alkyl-Y, —N(R³)(R^{3a}), —N(R³)—C₀-C₃alkyl-C₄-C₆heterocyclyl, —N(R³)—C₂-C₃alkyl-N(R³)(R^{3a}), —N(R³)—C₂-C₃alkyl-OR^{a}—, —N(R³)—C₀-C₃heteroalkyl-Y, —N(R³)—C(O)—O—C₀-C₃alkyl-Y, —N(R³)—C(S)—O—C₀-C₃alkyl-Y, —N(R³)—S(O)₂—C₀-C₃alkyl-Y, —N(R³)—C(O)—N(R³)—C₀-C₃alkyl-Y, —N(R³)—C(S)—N(R³)—C₀-C₃alkyl-Y, —N(R³)—C(O)—C₀-C₃alkyl-C₀-C₃heterocyclyl, —N(R³)—C(S)—C₀-C₃alkyl-C₀-C₃heterocyclyl, —N(R³)—C(O)—C₀-C₃alkyl-C₀-C₃heterocyclyl-Y, —N(R³)—C(S)—C₀-C₃alkyl-C₀-C₃heterocyclyl-Y and —N(R³)—S(O)₂—N(R³)—C₀-C₃alkyl-Y,
—C₀-C₆alkyl-, wherein when the —C₀-C₆alkyl is C₁-C₆alkyl it is optionally substituted with a substituent selected from the group consisting of —N(R³)—C(O)—C₀-C₃alkyl-Y, —N(R³)—C(O)—C₀-C₃alkyl-heterocyclyl and —N(R³)—C(O)—C₀-C₃alkyl-cycloalkyl,
—C₀-C₆alkyl-C(O)—N(R³)—C₀-C₃alkyl-, wherein when the —C₀-C₃alkyl is C₁-C₃alkyl it is optionally substituted with a substituent selected from the group consisting of —C(O)—N(R³)—C₀-C₃alkyl-Y, —C(O)-heterocyclyl, —C(O)—N(R³)(R^{3a}), aryl-aryl, aryl-heteroaryl, -heteroaryl-aryl, heteraryl-heteroaryl, heteroaryl, heterocyclyl-heteroaryl and heterocyclyl,
—C₀-C₆alkyl-heteroalkyl-C₀-C₆alkyl-C(O)—N(R³)—C₀-C₃alkyl-, wherein when the —C₀-C₃alkyl is C₁-C₃alkyl it is optionally substituted with a substituent selected from the group consisting of —C(O)—N(R³)—C₀-C₃alkyl-Y, —C(O)-heterocyclyl, —C(O)—N(R³)(R^{3a}), aryl-aryl, aryl-heteroaryl, -heteroaryl-aryl, heteraryl-heteroaryl, heteroaryl, heterocyclyl-heteroaryl and heterocyclyl,
—C₀-C₆alkyl-C(O)—N(R³)—C₀-C₃alkyl-, wherein when the —C₀-C₆alkyl is C₁-C₆alkyl it is optionally substituted with a substituent selected from the group consisting of —N(R⁷)(R^{7a}), —N(R³)(R^{3a}), —N(R³)—C(O)—C₀-C₃alkyl-Y, —N(R³)—C(O)—O—C₀-C₃alkyl-Y, —N(R³)—C(O)—N(R³)—C₀-C₃alkyl-Y and —N(R³)—S(O)₂—C₀-C₃alkyl-Y,
—C₀-C₆alkyl-heteroaryl-C₀-C₃alkyl-, wherein when the —C₀-C₃alkyl is C₁-C₃alkyl it is optionally substituted with a substituent selected from the group consisting of —C₀-C₃alkyl-N(R³)—C(O)—C₀-C₃alkyl-N(R³)(R^{3a}), —N(R³)—C(O)—C₀-C₃alkyl-Y, —N(R³)—C(O)—O—C₀-C₃alkyl-Y, —N(R³)—C(O)—N(R³)—C₀-C₃alkyl-Y and —N(R³)—S(O)₂—C₀-C₃alkyl-Y,
—C₀-C₆alkyl-aryl-C₀-C₃alkyl-, wherein when the —C₀-C₃alkyl is C₁-C₃alkyl it is optionally substituted with a substituent selected from the group consisting of —N(R³)—C(O)—C₀-C₃alkyl-Y, —N(R³)—C(O)—C₀-C₃alkyl-Y, —N(R³)—C(O)—O—C₀-C₃alkyl-Y, —N(R³)—C(O)—N(R³)—C₀-C₃alkyl-Y and —N(R³)—S(O)₂—C₀-C₃alkyl-Y,
—C₀-C₆alkyl-aryl-heteroaryl-C₀-C₃alkyl-, wherein when the —C₀-C₃alkyl is C₁-C₃alkyl it is optionally substituted with a substituent selected from the group consisting of —N(R³)—C(O)—C₀-C₃alkyl-Y, —N(R³)—C(O)—C₀-C₃alkyl-Y, —N(R³)—C(O)—O—C₀-C₃alkyl-Y, —N(R³)—C(O)—N(R³)—C₀-C₃alkyl-Y and —N(R³)—S(O)₂—C₀-C₃alkyl-Y,
—C₀-C₆alkyl-heteroaryl-heteroaryl-C₀-C₃alkyl-, wherein when the —C₀-C₃alkyl is C₁-C₃alkyl it is optionally substituted with a substituent selected from the group consisting of —N(R³)—C(O)—C₀-C₃alkyl-Y, —N(R³)—C(O)—C₀-C₃alkyl-Y, —N(R³)—C(O)—O—C₀-C₃alkyl-Y, —N(R³)—C(O)—N(R³)—C₀-C₃alkyl-Y and —N(R³)—S(O)₂—C₀-C₃alkyl-Y,
—C₀-C₆alkyl-heteroaryl-C₀-C₃alkyl-, wherein when the —C₀-C₃alkyl is C₁-C₃alkyl it is optionally substituted with a substituent selected from the group consisting of —N(R³)—C(O)—C₀-C₃alkyl-Y, —N(R³)—C(O)—C₀-C₃alkyl-Y, —N(R³)—C(O)—O—C₀-C₃alkyl-Y, —N(R³)—C(O)—N(R³)—C₀-C₃alkyl-Y and —N(R³)—S(O)₂—C₀-C₃alkyl-Y,
—C₀-C₆alkyl-O—C(O)—N(R³)—C₀-C₃alkyl-, wherein when the —C₀-C₃alkyl is C₁-C₃ alkyl it is optionally substituted with a group selected from —C(O)—OR^{a}, —C(S)—OR^{a}, —C(O)—N(R³)—C₁-C₃alkyl, —C(S)—N(R³)—C₁-C₃alkyl, —C(O)—N(R³)(R^{3a})—, —C(S)—N(R³)(R^{3a})—, —C(O)—N(R³)—C₀-C₃alkyl-aryl, —C(S)—N(R³)—C₀-C₃alkyl-aryl, —C(O)—N(R³)—C₀-C₃alkyl-heteroaryl, —C(S)—N(R³)—C₀-C₃alkyl-heteroaryl, —C(O)—N(R³)—C₀-C₃alkyl-cycloalkyl, —C(S)—N(R³)—C₀-C₃alkyl-cycloalkyl, —C(O)-heterocyclyl, —C(S)-heterocyclyl, —C(O)—N(R³)—C₀-C₃alkyl-Y, —C(S)—N(R³)—C₀-C₃alkyl-Y, —C(O)—N(R³)-heterocyclyl, —C(S)—N(R³)-heterocyclyl, —C(O)—N(R³)—C₀-C₃alkyl-heterocycloalkyl and —C(S)—N(R³)—C₀-C₃alkyl-heterocycloalkyl;
—C₀-C₆alkyl-O—C(S)—N(R³)—C₀-C₃alkyl-, wherein when the —C₀-C₃alkyl is C₁-C₃ alkyl it is optionally substituted with a group selected from —C(O)—OR^{a}, —C(S)—OR^{a}, —C(O)—N(R³)—C₁-C₃alkyl, —C(S)—N(R³)—C₁-C₃alkyl, —C(O)—N(R³)(R^{3a})—, —C(S)—N(R³)(R^{3a})—, —C(O)—N(R³)—C₀-C₃alkyl-aryl, —C(S)—N(R³)C₀-C₃alkyl-aryl, —C(O)—N(R³)—C₀-C₃alkyl-heteroaryl, —C(S)—N(R³)—C₀-C₃alkyl-heteroaryl)C₀-C₃alkyl-cycloalkyl, —C(S)—N(R³)—C₀-C₃alkyl-cycloalkyl, —C(O)-heterocyclyl, —C(S)-heterocyclyl, —C(O)—N(R³)—C₀-C₃alkyl-Y, —C(S)—N(R³)—C₀-C₃alkyl-Y, —C(O)—N(R³)-heterocyclyl, —C(S)—N(R³)-heterocyclyl, —C(O)—N(R³)—C₀-C₃alkyl-heterocycloalkyl and —C(S)—N(R³)—C₀-C₃alkyl-heterocycloalkyl; and
—C₁-C₃alkyl-N(R³)—C(O)—C₁-C₇alkyl-, wherein the C₁-C₃alkyl is optionally substituted with —C(O)N(R³)—C₁-C₃alkyl-A¹³ and the C₁-C₇alkyl is optionally substituted with a substituent selected from the group consisting of —N(R³)—C(O)—O—C₁-C₃alkyl-A^{1b}, —N(R³)—C(O)—C₁-C₃alkyl-A^{1b}, —N(R³)—S(O)₂—C₁-C₃alkyl-A^{1b}, —N(R³)—S(O)₂—N(R³)—C₁-C₃alkyl-A^{1b}, —N(R³)—C(O)—N(R³)—C₁-C₃alkyl-A^{1b} and —N(R³)—S(O)₂—N(R³)—C₁-C₃alkyl-A^{1b}, wherein
A^{1a} and A^{1b} are independently selected from the group consisting of alkyl, alkenyl and a protecting group; or
A^{1a} and A^{1b} together via a —C₂-C₆alkylene-, —C₂-C₆alkenylene-, —C₂-C₆alkynylene- or —C₀-C₃alky-heteroaryl-C₀-C₃alky-linker, form an optionally substituted ring.

7. The compound according to claim 1, wherein L is selected from the group consisting of
—C₁-C₆alkyl-N(R³)—C₀-C₃alkyl-, wherein the C₁-C₆alkyl is optionally substituted with a substitutent selected from the group consisting of —C₁-C₄ alkyl-OR^{a}, —C₁-C₆ alkyl-N(R³)(R^{3a})—, —C₀-C₄ alkyl-C(O)OR^{a} and —C₀-C₃alkyl-C(O)—N(R³)(R^{3a}),
—C₀-C₆alkyl-N(R³)—C(O)—C₀-C₃alkyl-, wherein the C₁-C₆alkyl is optionally substituted with a substituent selected from the group consisting of —C₁-C₄alkyl-O(R^{a})—, —C₀-C₆alkyl-C(O)O(R^{a})— and —C₁-C₆alkyl-N(R³)(R^{3a})—, and
—C₀-C₆alkyl-C(O)—N(R³)—C₀-C₃alkyl-, wherein the C₁-C₆alkyl is optionally substituted with a substituent selected from the group consisting of —C₀-C₆alkyl-O(R^{a})—, —C₀-C₆alkyl-C(O)O(R^{a})—, —C₀-C₃alkyl-C(O)—N(R³)(R^{3a}) and —C₀-C₆alkyl-N(R³)(R^{3a})—.

8. The compound according to claim 1, wherein L is selected from the group consisting of
—C₀-C₆alkyl-N(R³)—C(O)—C₁-C₇alkyl-, wherein the C₁-C₇alkyl is optionally substituted with a substituent selected from the group consisting of —N(R⁷)(R^{7a}), —N(R³)C(O)—C₀-C₃alkyl-heterocyclyl, —N(R³)—C(O)—C₀-C₆alkylaryl-R^{a}, —N(R³)—C(O)—C₁-C₆alkyl-R^{a} and —N(R³)—C(O)—O—C₀-C₃alkyl-Y wherein heterocyclyl is optionally substituted,
—C₀-C₆alkyl-C(O)—N(R³)—C₁-C₇alkyl-, wherein the C₁-C₆alkyl is optionally substituted with —N(R⁷)(R^{7a}),
—C₀-C₆alkyl-C(O)—N(R³)—C₁-C₇alkyl-, wherein the C₁-C₇alkyl is optionally substituted with a substituent selected from the group consisting of aryl-aryl, aryl-heteroaryl, heteroaryl-heteroaryl, heteroaryl-aryl and heteroaryl, and
—C₁-C₃alkyl-N(R³)—C(O)—C₁-C₇ alkyl-, wherein the C₁-C₃alkyl is optionally substituted with —C(O)N(R³)—C₁-C₃alkyl-Ala and the C₁-C₇alkyl is optionally substituted with a substituent selected from the group consisting of —N(R³)—C(O)O—C₁-C₃alkyl-A^{1b}, —N(R³)C(O)—C₁-C₃alkyl-A^{1b}, —N(R³)—S(O)₂—C₁-C₃alkyl-A^{1b}, —N(R³)—C(O)—N(R³)—C₃alkyl-A^{1b} and —N(R³)— S(O)₂—N(R³)—C₁-C₃alkyl-A^{1b}, wherein
A₁ₐ and A_{1b} are independently selected from the group consisting of alkyl, alkenyl and a protecting group; or
A₁ₐ and A_{1b} together via a —C₂-C₆alkylene, —C₂-C₆alkenylene, —C₂-C₆alkynylene, —C₀-C₃alkyl-heteroaryl-C₀-C₃alkyl-linker or —C₀-C₃alkyl-aryl-C₀-C₃alkyl-linker, form an optionally substituted ring, and

9. The compound according to claim 1, wherein L is a selected from the group consisting of
—C₀-C₇alkyl-N(R₃)—C(O)-heterocyclyl-C₀-C₆alkyl-, wherein a C₁-C₇alkyl is optionally substituted with —C₀-C₃alkyl-C(O)OR^{a} or —C₁-C₃alkyl-OR^{a}, and
—C₀-C₇alkyl-O—C(O)-heterocyclyl-C₀-C₆alkyl-, wherein a C₁-C₇alkyl is optionally substituted with —C₀-C₃alkyl-C(O)OR^{a} or —C₀-C₃alkyl-OR^{a}.

10. The compound according to claim 1, wherein L is selected from the group consisting of a covalent bond, —(CH₂)₁₋₄—, —(CH₂)₀₋₄—(CR³═CR³)—(CH₂)₀₋₄—, —(CH₂)₀₋₄—(C≡C)—(CH₂)₀₋₄—, —(CH₂)₀₋₃ N(R³)C(O)—, —(CH₂)₀₋₃—C(O)N(R³)—, —(CH₂)₀₋₃ N(R³)C(O)—(CR^{a}═CR^{a})—, —(CH₂)₀₋₃—N(R³)—(CH₂)₂₋₄ N(R³)C(O)—, —(CH₂)₀₋₃—O—(CH₂)₂₋₄—N(R³)C(O)—, —(CH₂)₀₋₃C(O)—(CH₂)₀₋₃—, —(CH₂)₀₋₃—(CR^{a}═CR^{a})—C(O)—(CH₂)₀₋₃—, —(CH₂)₀₋₃C(O)—(CR^{a}═CR^{a})—, —(CH₂)₀₋₃—, —C₀-C₆alkyl-N(R³)—C(O)-heterocyclyl-C₀-C₃alkyl-, —C₀-C₆alkyl-S(O)₂-heterocyclyl-C₀-C₃alkyl-, —(CH₂)₀₋₃—S(O)₂—N(R³)—(CH₂)₀₋₃—, —(CH₂)₀₋₃ N(R³)—S(O)₂—(CH₂)₀₋₃—, —(CH₂)₀₋₃N(R³)—(CH₂)₀₋₃—, —(CH₂)₀₋₃N(R³)—(CH₂)₁₋₃—(CR^{a}═CR^{a})—, —(CH₂)₀₋₃C═N—O—(CH₂)₀₋₃—, —(CH₂)₀₋₃N(R⁷)—(CH₂)₀₋₃—, —(CH₂)₀₋₃S—(CH₂)₀₋₃—, —(CH₂)₀₋₃O—(CH₂)₀₋₃—, —(CH₂)₀₋₃S(O)—(CH₂)₀₋₃—, —(CH₂)₀₋₃S(O)₂—(CH₂)₀₋₃—, —(CH₂)₀₋₃CH═CH—(CH₂)₂₋₃—, —(CH₂)₀₋₃N(R³)—C(O)—N(R³)—(CH₂)₀₋₃, —(CH₂)₀₋₃N(R³)—C(O)—O—(CH₂)₀₋₃, —(CH₂)₀₋₃O—C(O)—N(R³)—(CH₂)₀₋₃—, —(CH₂)₀₋₃ N(R³)—C(O)—N(R₃)—S(O)₂—(CH₂)₀₋₃—, and —(CH₂)₀₋₃N(R³)—C(O)—N(R³)—C(O)—(CH₂)₀₋₃—.

11. The compound according to claim 1, wherein B₁, B₂ and B₃ are independently selected from the group consisting of D-Gly, L-Gly, D-Pro, L-Pro, D-Tyr, L-Tyr, D-Tyr(OR^{a}), L-Tyr(OR^{a}), D-Phe, L-Phe, D-PheR₄, L-PheR₄, D-Aib, L-Aib, D-Ala, L-Ala, D-ProR₃, L-ProR₃, D-Ile, L-Ile, D-Leu, L-Leu D-PheR₃, L-PheR₃, D-Pip and L-Pip.

12. The compound according to claim 1, wherein each alkyl, alkenyl, alkynyl, heteroalkyl, benzyl and heterocyclyl moiety of R⁷ and R^{7a} is independently optionally substituted with one or more substituents selected from the group consisting of oxo, —OH, —CN, C₁-C₆alkyl, C₁-C₆alkoxy, —NO₂, —N(R³)(R^{3a}), halo, —SH and mono- to per-halogenated C₁-C₆alkyl.

13. The compound according to claim 1, wherein Y is selected from the group consisting of aromatic polycycle, non-aromatic polycycle, mixed aryl and non-aryl polycycle, polyheteroaryl, non-aromatic polyheterocycle, mixed aryl and non-aryl polyheterocycle, each of which is optionally substituted.

14. The compound according to claim 1, wherein Y is selected from the group consisting of aryl, aryl-aryl, heteroaryl, aryl-heteroaryl, heteroaryl-aryl, cycloalkyl, heterocyclyl and heterocyclyl-heteroaryl, each of which is optionally substituted.

15. The compound according to claim 1, wherein R^{a}, R^{b} and R^{c} are independently selected from the group consisting of —H, C₁-C₃alkyl, C₃-C₆cycloalkyl, aryl, heteroaryl, and aryl-C₁-C₃alkyl-.

16. The compound according to claim 1, wherein R^{a} and R^{b} together with the nitrogen atom to which they are attached form a 3 to 9-membered heterocyclyl, heteroaryl, or heterocyclyl-aryl, wherein each of the heterocyclyl, heteroaryl and heterocyclyl-aryl is optionally substituted;

17. The compound according to claim 1, wherein R³ and R^{3a} are independently selected from the group consisting of —H, OH, C₁-C₆alkyl, C₃-C₆cycloalkyl, —C(O)CF₃, —C(O)H, —C₁-C₄alkyl-C(O)OR^{a}, heterocyclyl, —C₂-C₄alkyl-OR^{a}, C₂-C₄alkylene; C₂-C₆alkenyl, C₂-C₆ hydroxyalkyl-C₁-C₆ alkylaryl, aryl, —C₀-C₆alkylheteroaryl, and —C₁-C₃alkyl-C(O)NR-heteroaryl.

18. The compound according to claim 17, wherein R³ and R^{3a} are independently selected from the group consisting of —C₁-C₆alkylaryl, t-butyl, benzyl and aryl.

19. The compound according to claim 17, wherein R³ and R^{3a} are independently selected from the group consisting of ethanol, tetrahydro-2H-pyran, phenyl and benzyl.

20. The compound according to claim 17, wherein R³ and R^{3a} are independently C₁-C₄ alkyl.

21. The compound according to claim 1, wherein in a —N(R³)(R^{3a}) group, the R³ and the R^{3a} together with the nitrogen atom to which they are attached optionally form a ring selected from the group consisting of morpholinyl, piperazinyl, piperidinyl, pyrrolydinyl, and azetidinyl.

22. The compound according to claim 1, wherein R⁴ is selected from the group consisting of —H, —CH₃, —S(O)₂—N(R³)(R^{3a}), —SO₃H, —O—C₂-C₄alkyl-heterocyclyl, —O—C₀-C₄alkyl-aryl, —O—C₀-C₄alkyl-heteroaryl, —O—C(O)N(R³)—C₀-C₄alkyl-aryl, —O—C(O)N(R³)—C₀-C₄alkyl-heteroaryl, —O—C₀-C₄alkyl-heterocyclyl-aryl, —O—C₀-C₄alkyl-heterocyclyl-heteroaryl, —N(R³)—C₂-C₄alkyl-heterocyclyl, —(CH₂)₀₋₄OR^{a}, —(CH₂)₀₋₄N(R³)(R^{3a}), —F, —Cl, —Br, —CF₃, —CN, —CH₂OH, —OH, —OCH₃, —NO₂, Ph, aryl, heteroaryl, —N(R³)C(O)CH₂R³, —N(R³)SO₂CH₂R^{a}, —O(CH₂)₂₋₄N(R³)(R^{3a}), —SR^{a}, —S(O)CH₂R^{a}, —SO₂CH₂R^{a}, —(CH₂)₀₋₄C(O)OR^{a}, —CH═CHC(O)OR^{a}, —CH═CHC(O)N(R³)(R^{3a}), —N(R³)C(O)CF₃ and —N(R³)(CH₂)₂N(R³)(R^{3a}).

23. The compound according to claim 1, wherein L is selected from the group consisting of 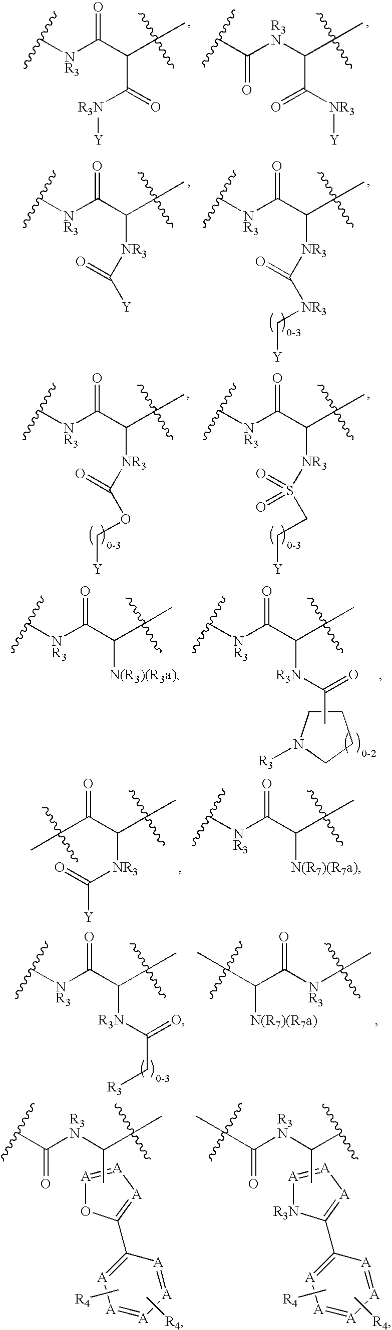 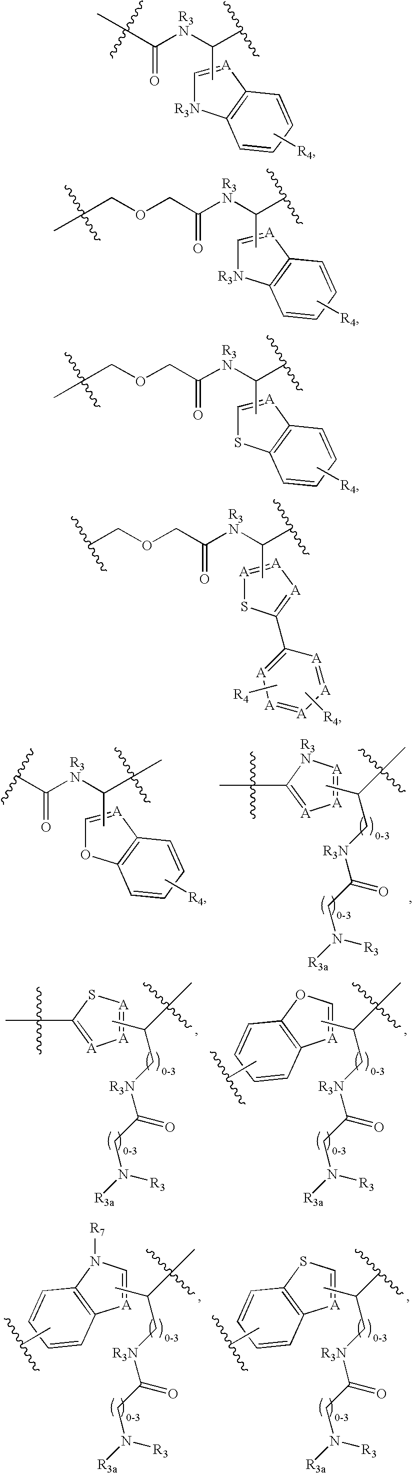 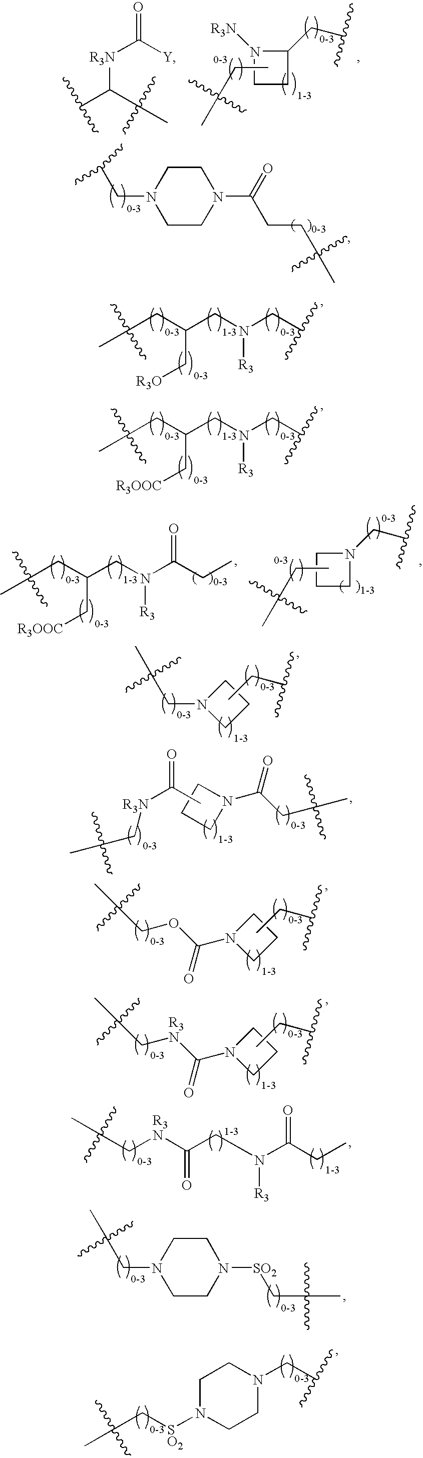 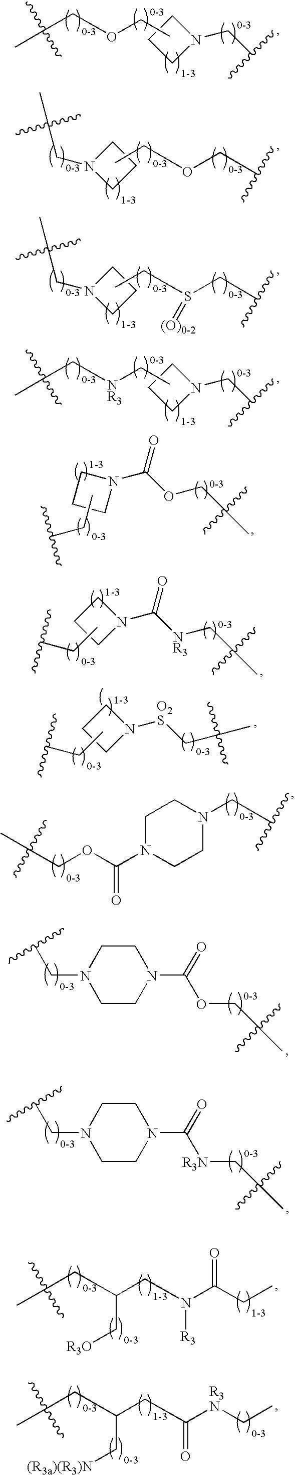 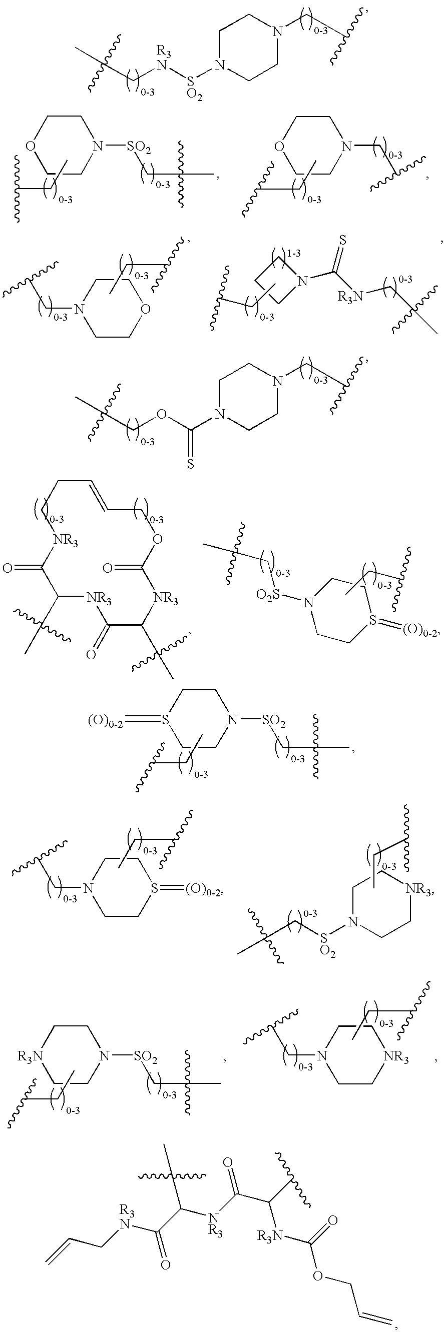 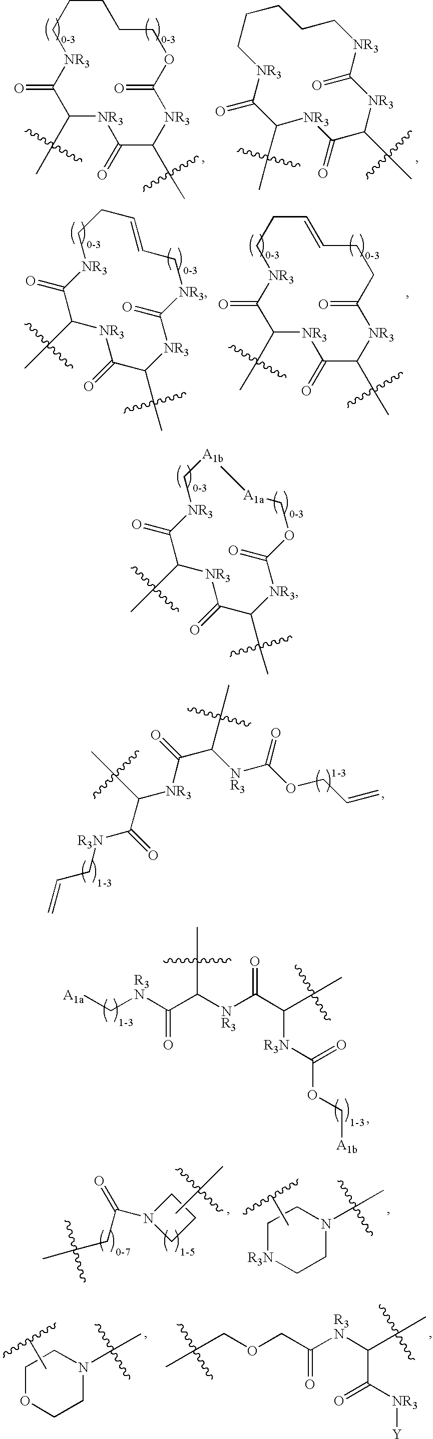 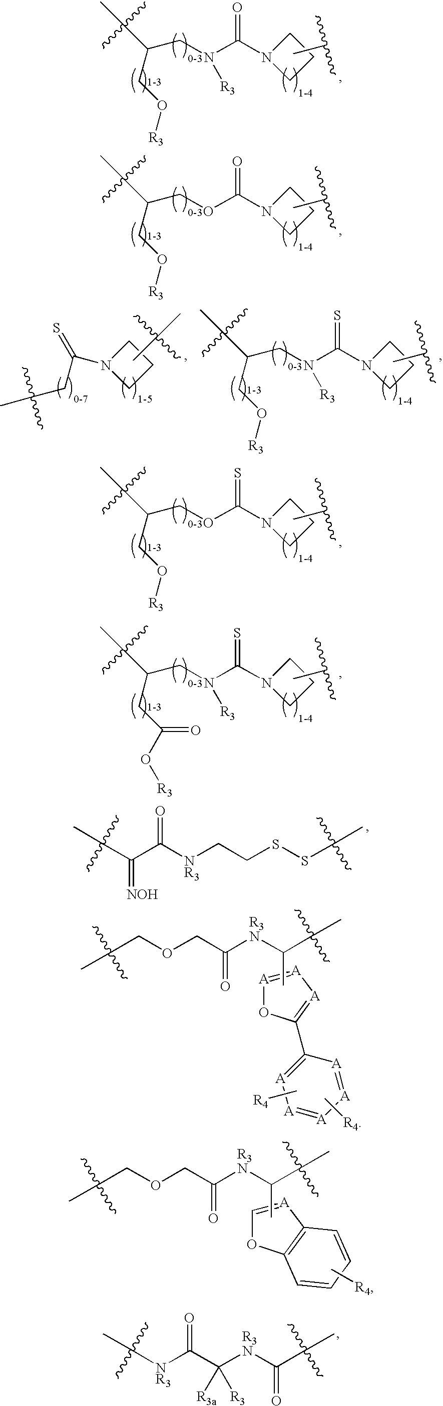 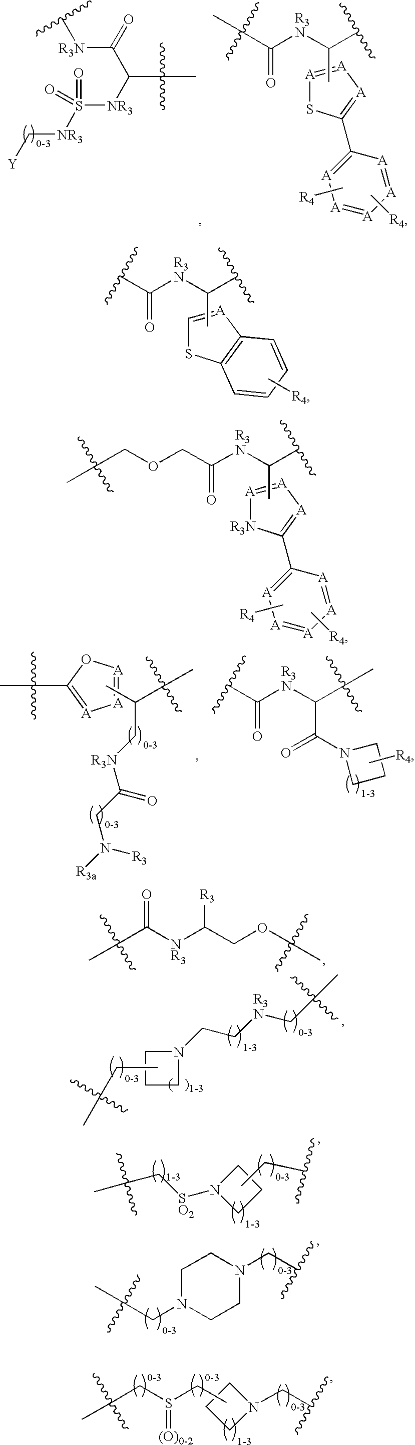 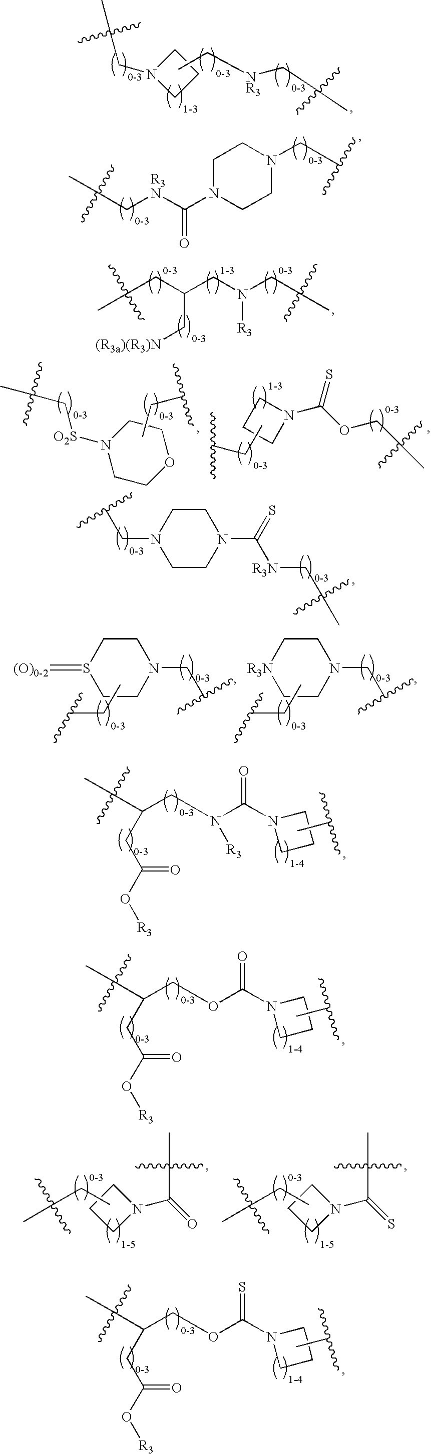 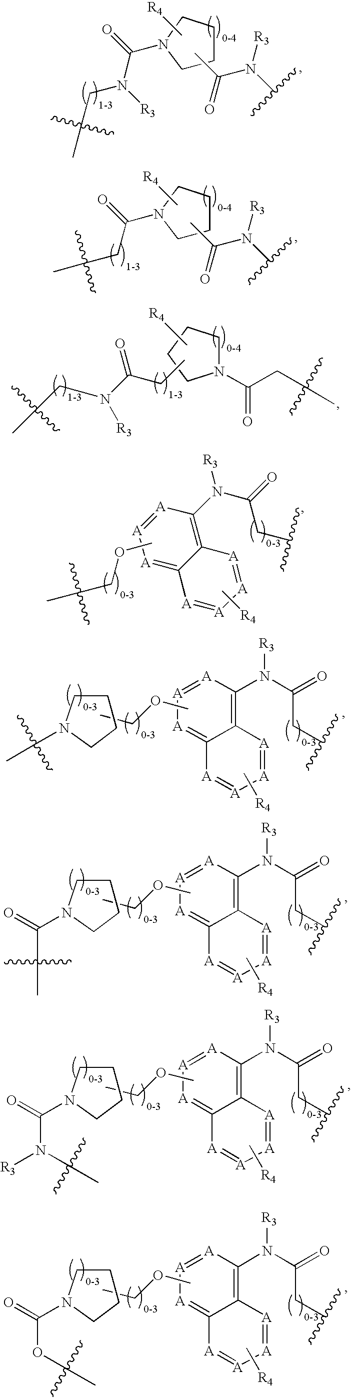 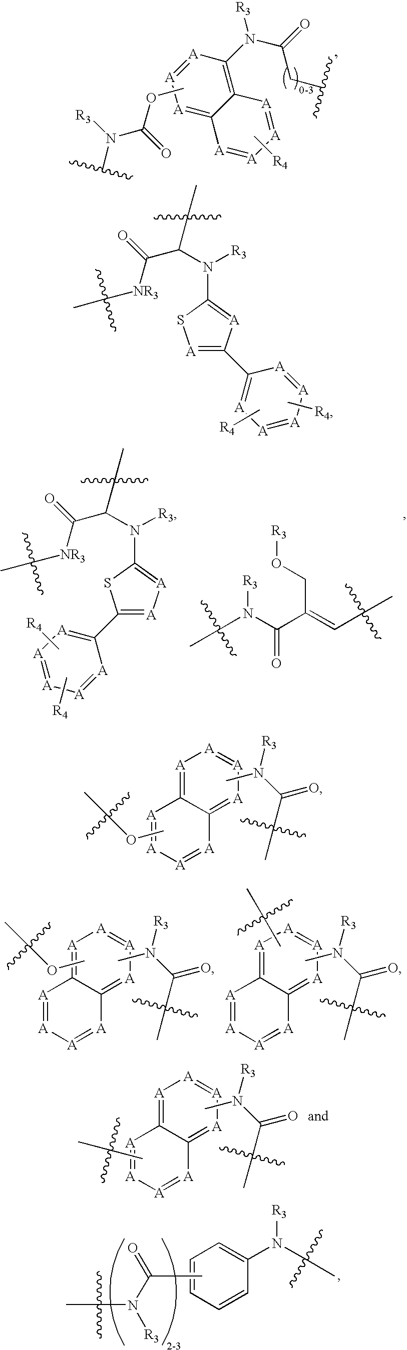
wherein
A₁ₐ and A_{1b} are independently selected from the group consisting of alkyl, alkenyl and protecting group and
A is N, CH or C (when A is attached to Y or Z), wherein there may be 0, 1, 2 or 3 nitrogen.

24. The compound according to claim 1, wherein Z is selected from the group consisting of 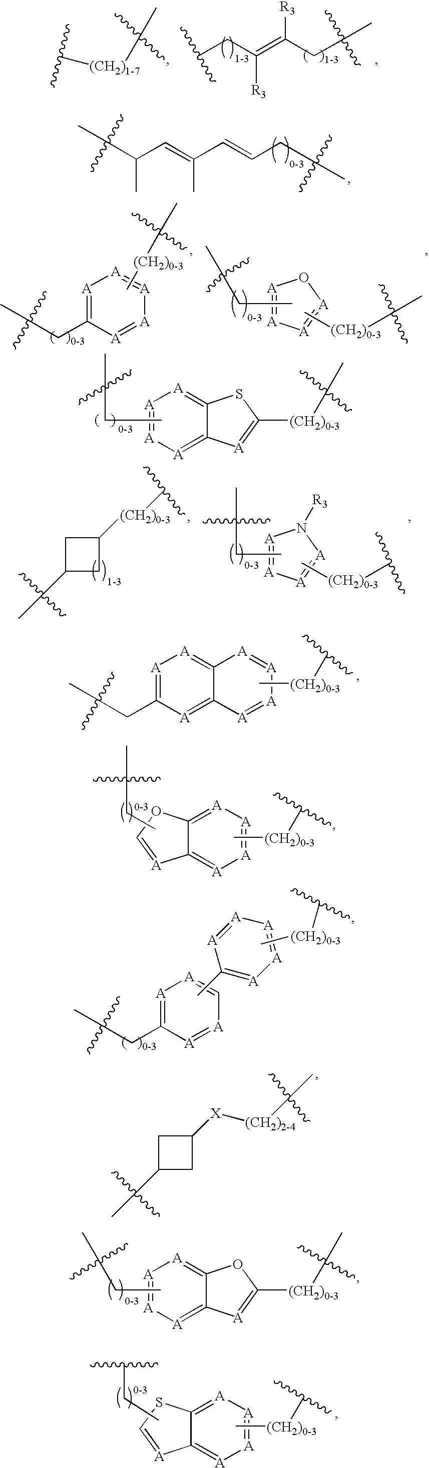 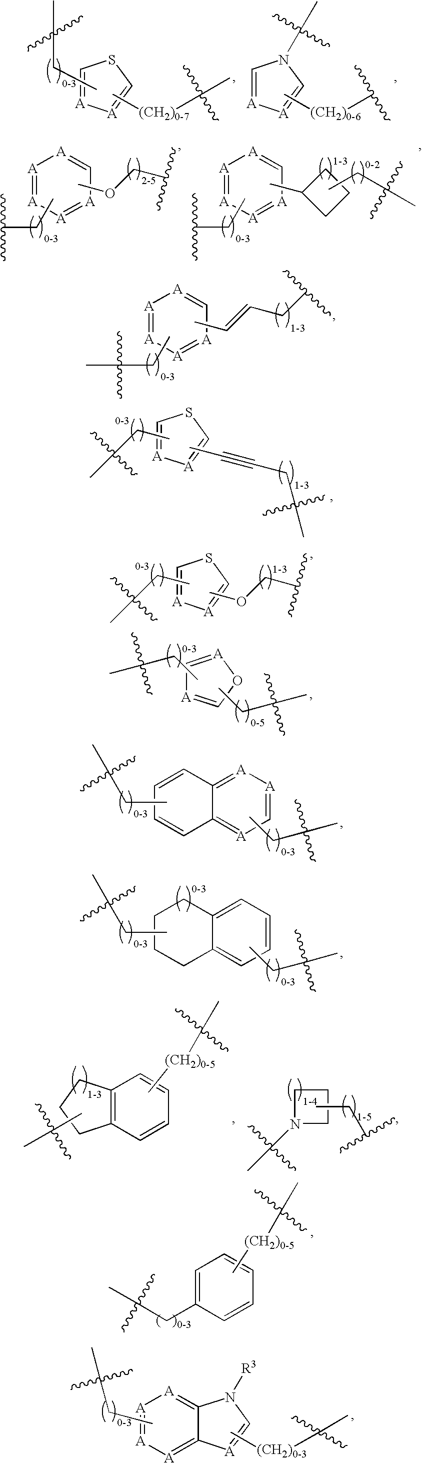 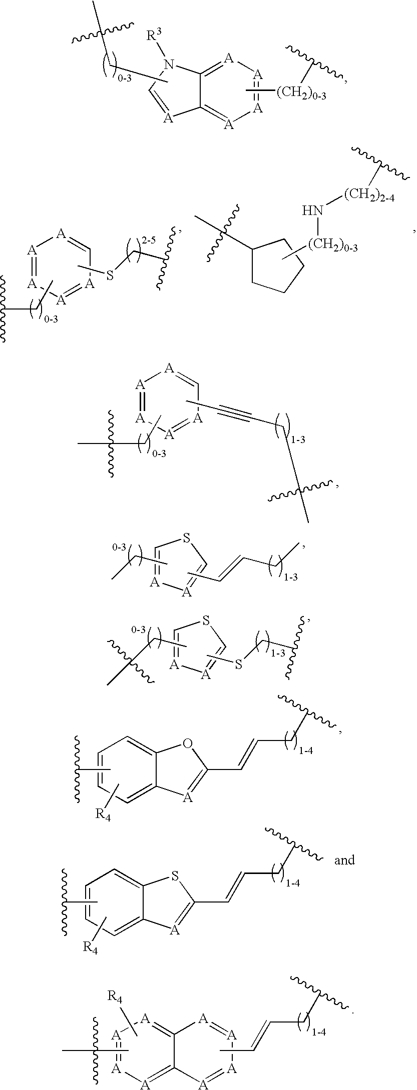
wherein A is nitrogen, —CH═ or —C(R⁴)═, wherein there may be 0, 1, 2 or 3 nitrogen.

25. The compound according to claim 1, wherein Y is selected from the group consisting of 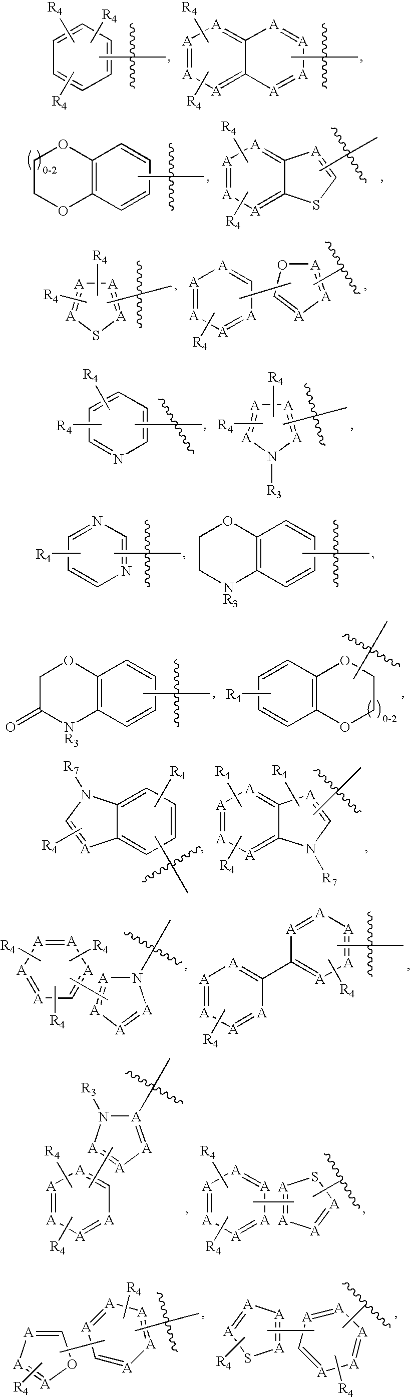 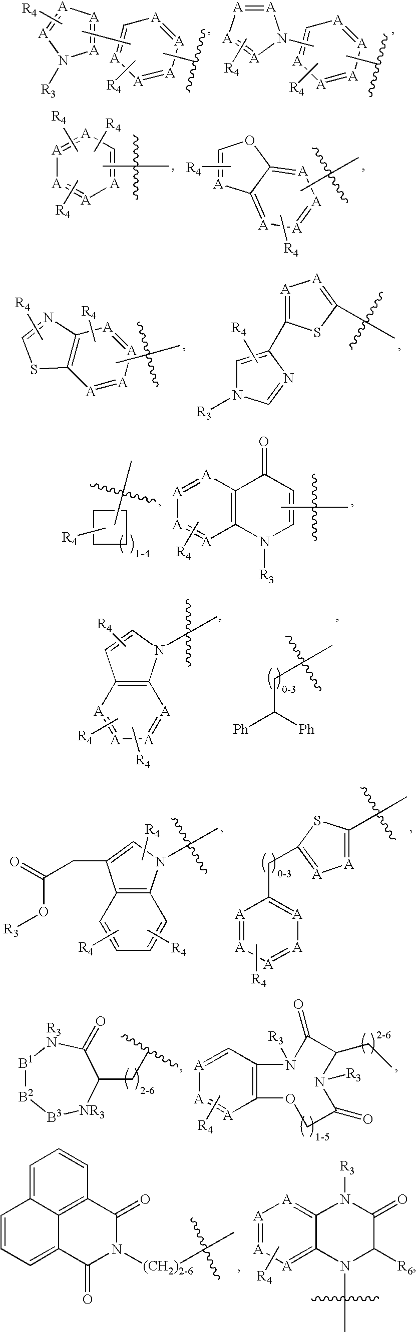 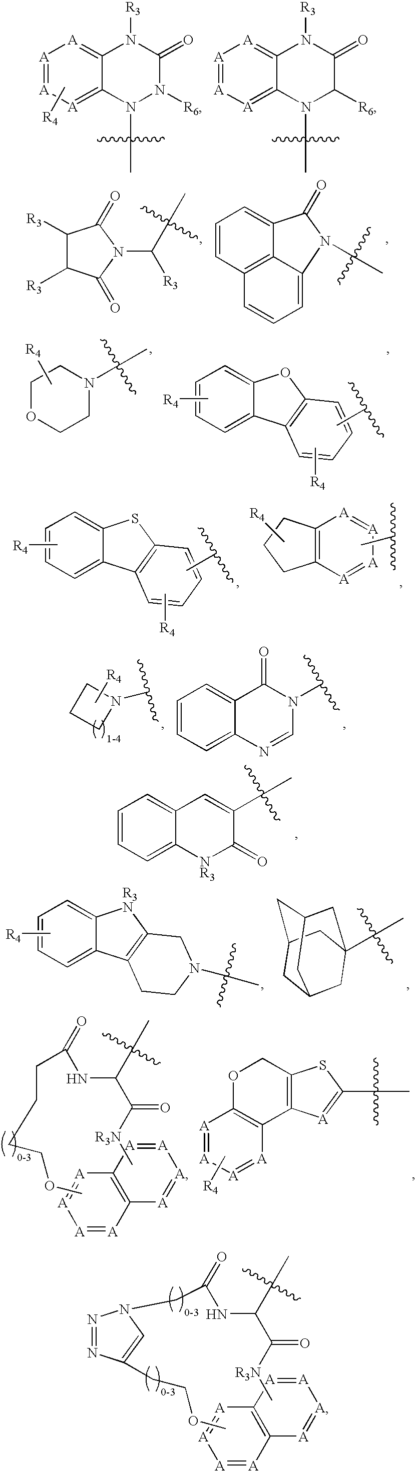 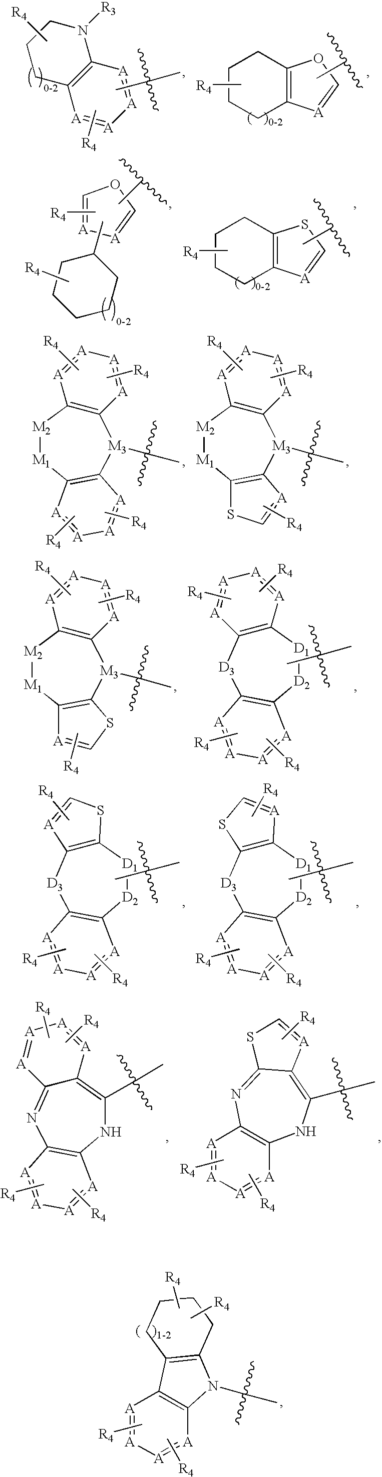 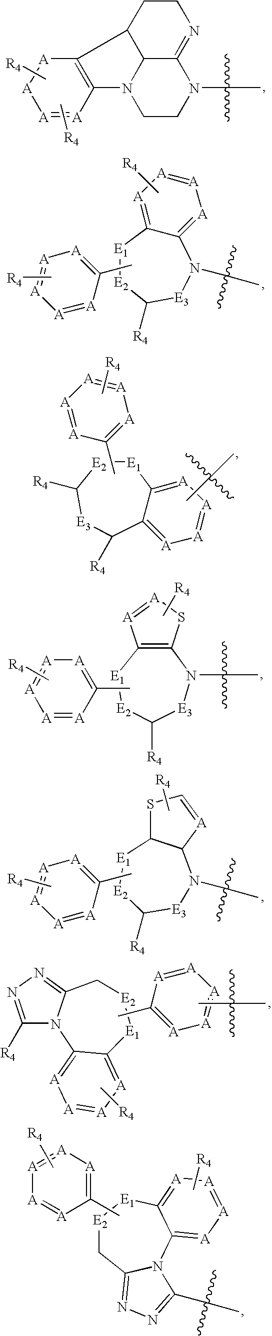 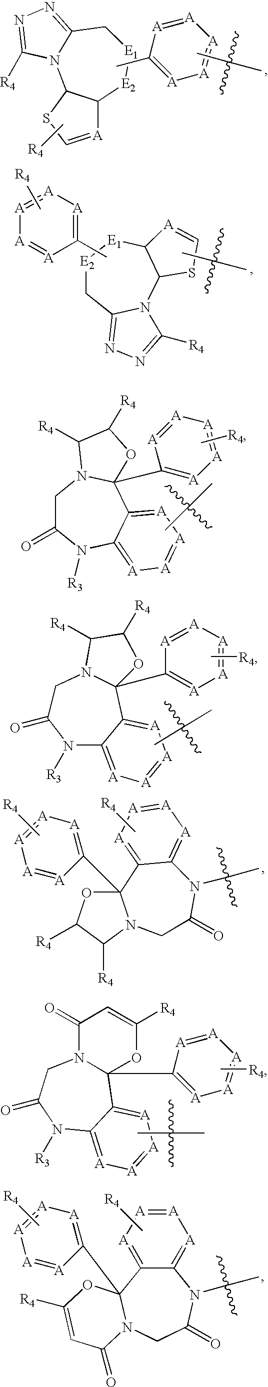 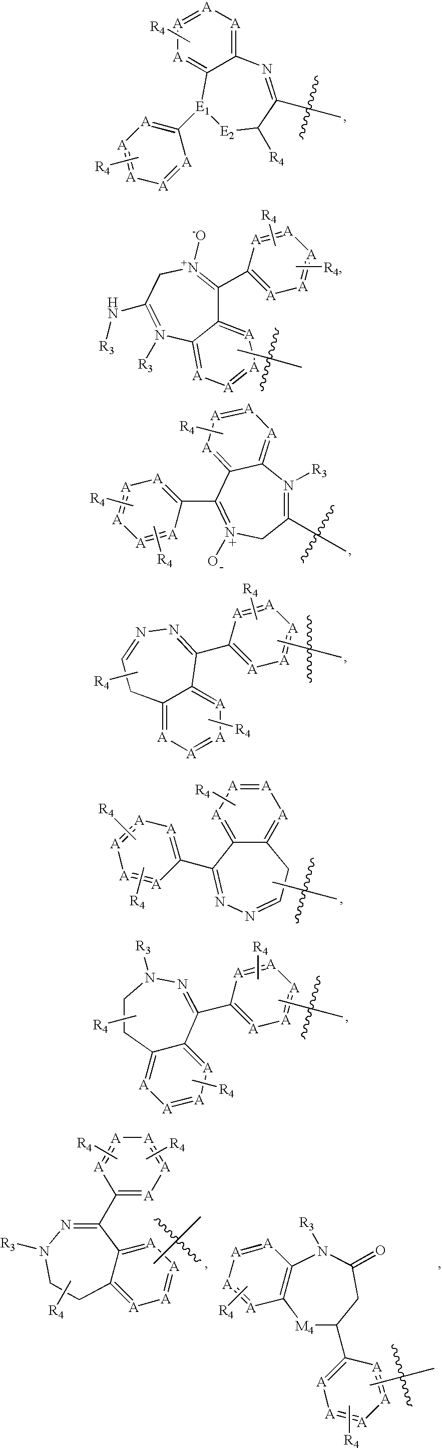 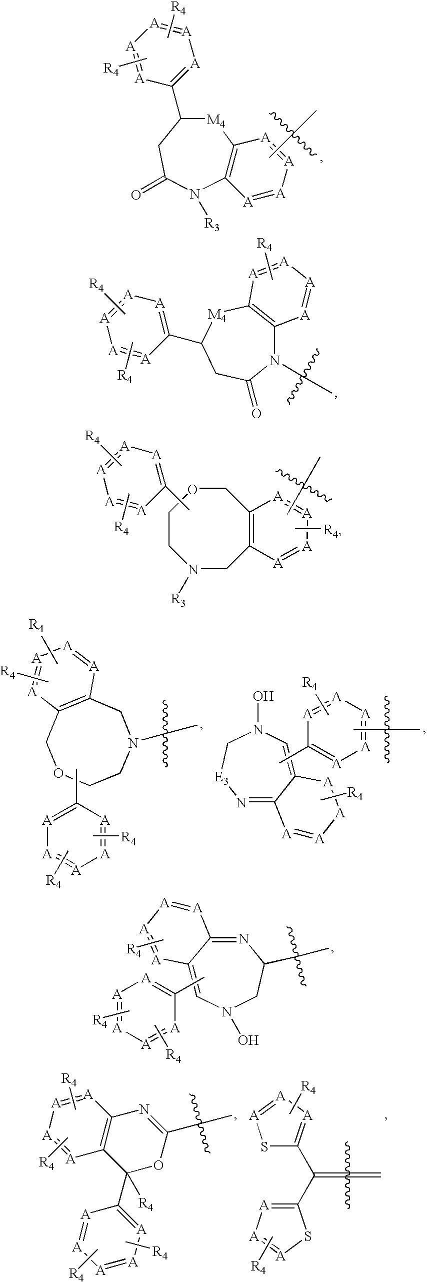 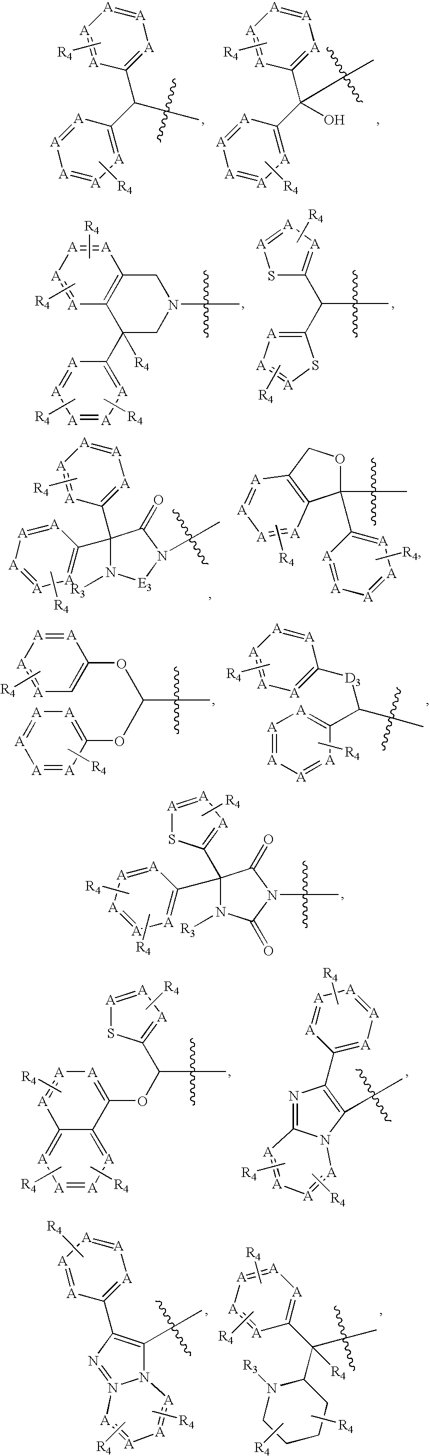 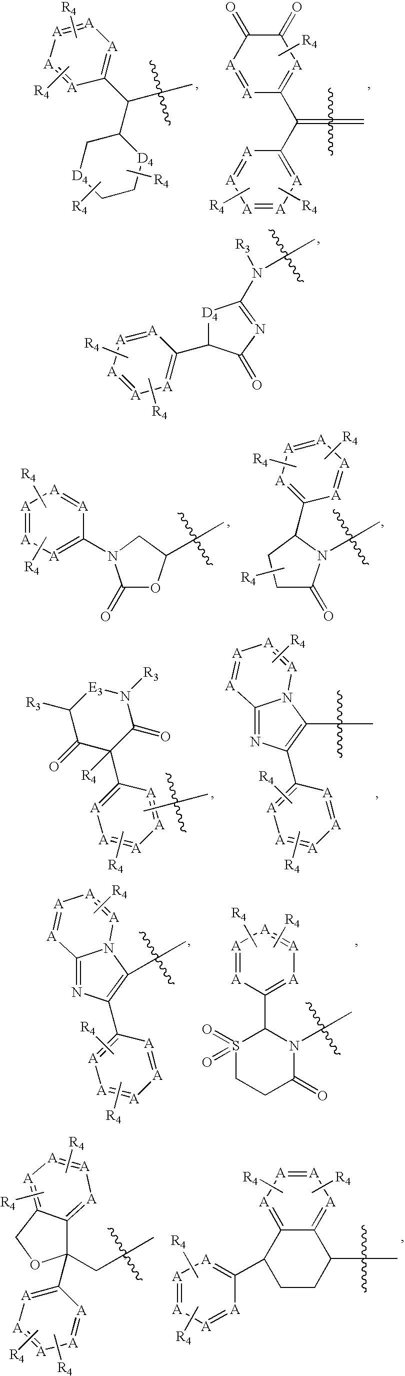 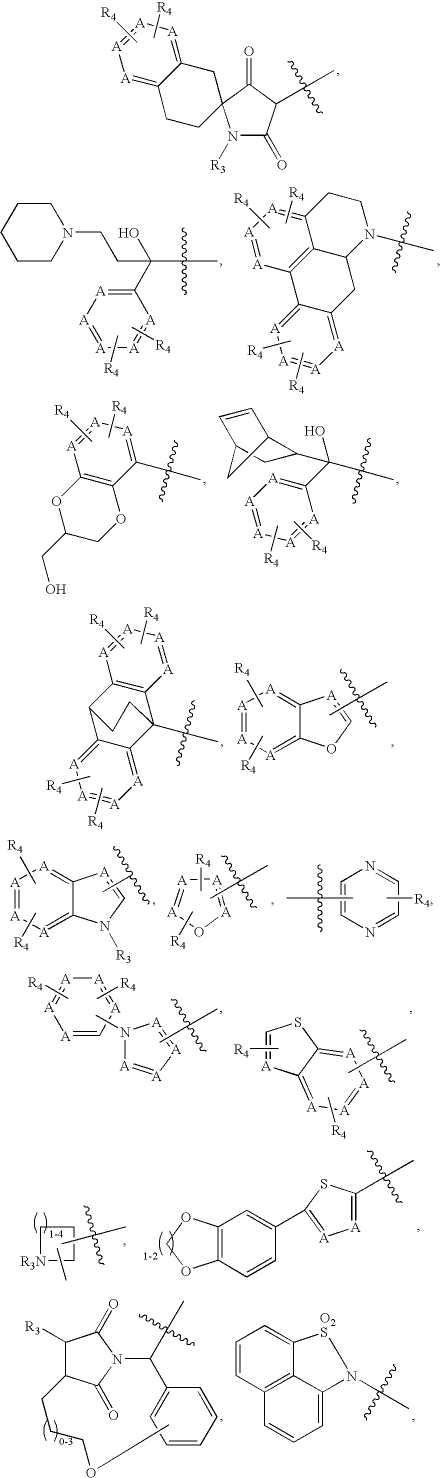 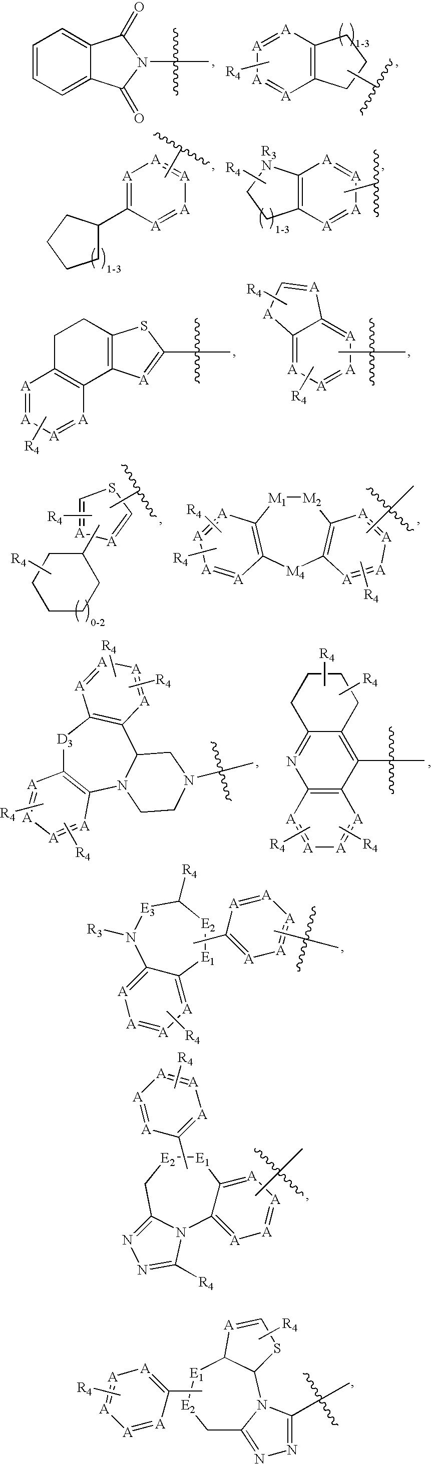 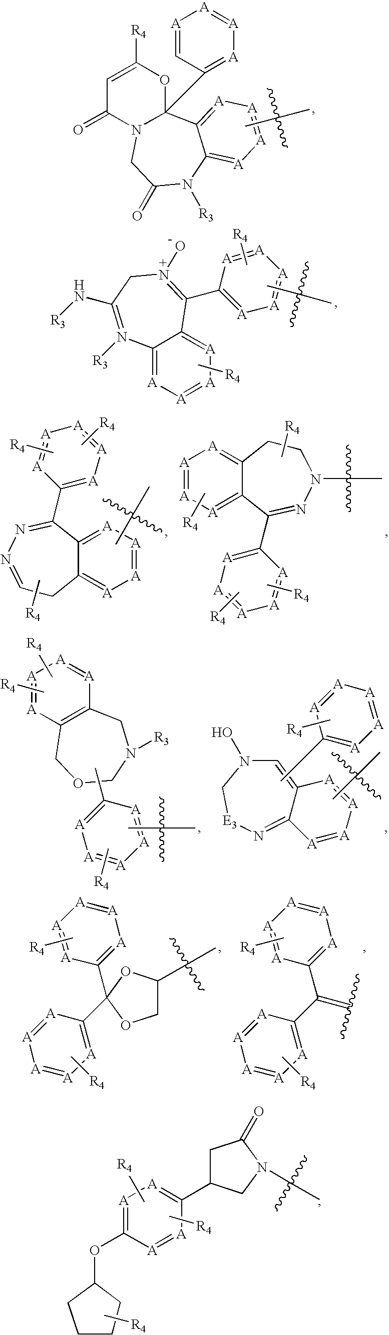 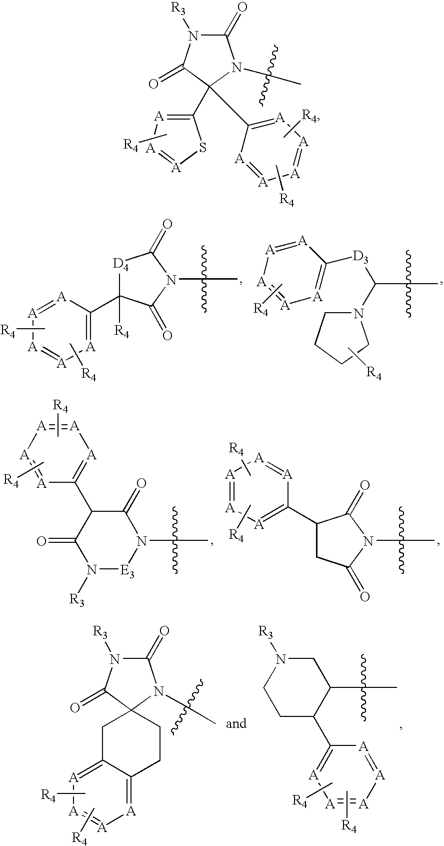
wherein
A is nitrogen, —C(H)═ or —C(R⁴)═, wherein there may be 0, 1, 2 or 3 nitrogen;
B¹, B² and B³ are each independently a natural or synthetic amino acid;
M₁-M₂ is selected from the group consisting of a covalent bond, —N(R³)CH₂—, —CH₂N(R³)—, —S(O)₀₋₂—CH₂—, —CH₂S(O)₀₋₂—, —O—CH₂—, —CH₂—O—, —C(O)N(R^{a})—, —N(R³)C(O)—, —SO₂N(R³)—, —N(R³)SO₂—, —CH(R^{a})CH₂—, —CH₂CH(R^{a})—, —N═C(R^{a})—, —C(R^{a})═N—, —CH₂—CH₂—, —CH═CH—, —CH(R^{a})—CH(R^{a})—, —C(R^{a})═C(R^{a})—, —CH₂—, —C(R^{a})(R^{a})—, —S—, —N(R³)— and absent;
M₃ is selected from the group consisting of
M₄ is selected from the group consisting of 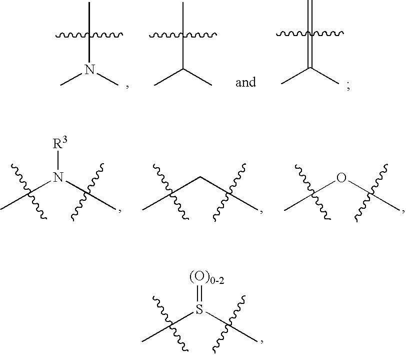
and covalent bond;
wherein, when M₁-M₂ is covalent bond, M₄ is 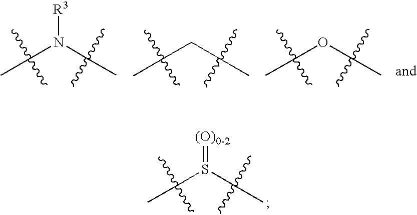
D₁-D₂ is selected from the group consisting of a 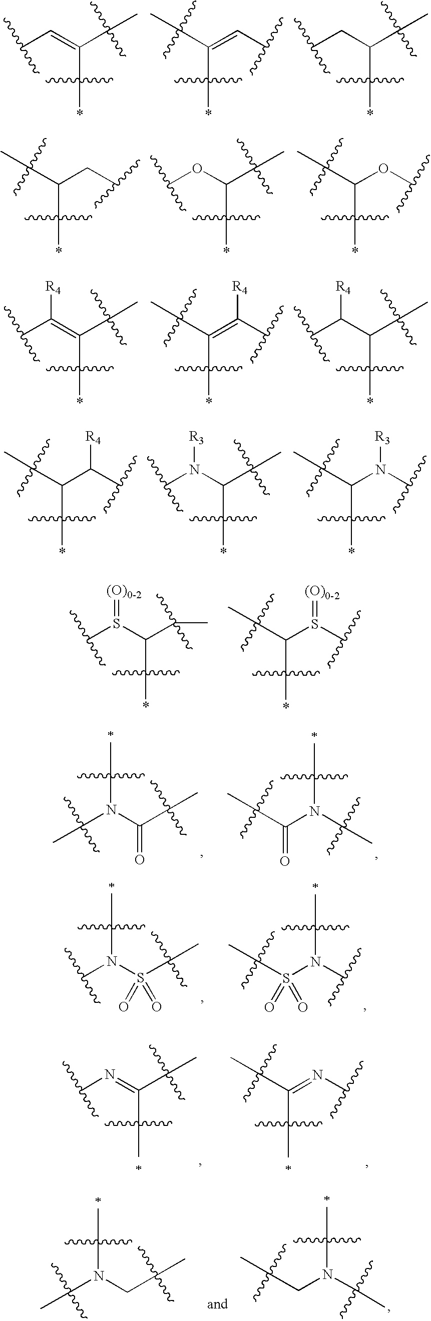
wherein, * represents the point of attachment to Q;
D₃ is selected from the group consisting of a covalent bond, 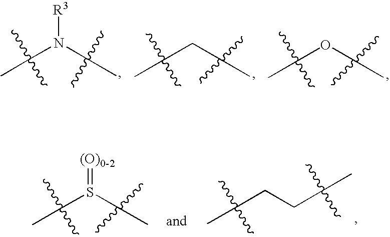
wherein the 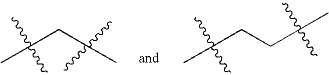
are
optionally substituted
D₄ is selected from the group consisting of 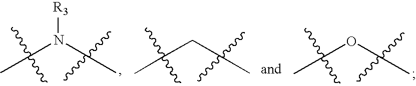
wherein the 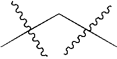
is optionally substituted,
E₁-E₂ is selected from the group consisting of 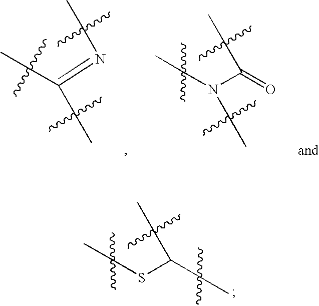
E₃ is selected from the group consisting of —C(O)—, —C(S)—, —CH₂—, —C(OH)₂— and —C═NR₃—; and
R⁶ is selected from the group consisting of —H, —C₁-C₆alkyl, —C₂-C₆alkenyl, —C₂-C₆alkynyl, —C₁-C₆heteroalkyl, heterocyclyl-C₀-C₆alkyl-, aryl-C₀-C₆alkyl-, heteroaryl-C₀-C₆alkyl-, C₃-C₆cycloalkyl-C₀-C₆alkyl-, N(R³)(R^{3a})—C₁-C₆alkyl-, N(R³)(R^{3a})—C(O)—C₁-C₆alkyl- and N(R³)(R^{3a})—C(S)—C₁-C₆alkyl-, wherein each alkyl, alkenyl, alkynyl, heteroalkyl, cycloalkyl, aryl, heteroaryl, or heterocyclyl moiety is optionally substituted.

26. The compound according to claim 25, wherein each alkyl, alkenyl, alkynyl, heteroalkyl, cycloalkyl, aryl, heteroaryl, and heterocyclyl moiety of R₆ is independently optionally substituted with one or more groups independently selected from R⁴.

27. The compound according to claim 25, wherein R⁶ is selected from the group consisting of

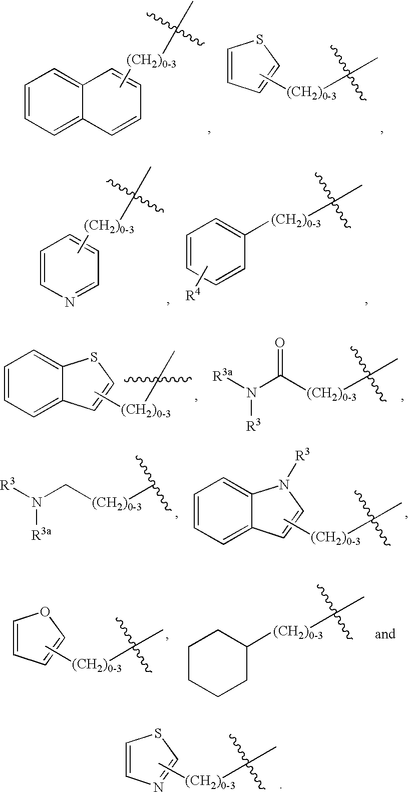

28. The compound according to claim 1, wherein R⁷ is selected from the group consisting of —H, optionally substituted C₁-C₆ alkyl, —(CH₂)₂₋₄OR^{a}, —OMe, —(CH₂)₂₋₄N(R³)(R^{3a}), —C(O)Ot-butyl, —C(O)O-benzyl, —(CH₂)₂-morpholinyl or —(CH₂)₂-piperazynnyl.

29. The compound according to claim 1, wherein
W and M are nitrogen;
R^{a}, R^{b} and R^{c} are —H;
Z is —C₁-C₈ alkyl-;
L is covalent bond, —C₀-C₆ alkyl-N(R³)C(O)—C₀-C₃ alkyl- or —C₀-C₆ alkyl-C(O)N(R³)—C₀-C₃ alkyl-; and
Y is selected from the group consisting of aryl, heteroaryl, aryl-aryl, heteroaryl-aryl, aryl-heteroaryl and polycycle, wherein each alkyl, aryl, heteroaryl and polycycle group is optionally substituted.

30. The compound according to claim 29, wherein L is a covalent bond or —C₀-C₆ alkyl-N(R³)C(O)—C₀-C₃ alkyl-.

31. The compound according to claim 29, wherein the alkyl, aryl, heteroaryl and polycycle groups are optionally substituted with aryl-C₀-C₆alkyl-O—, heteroaryl-C₀-C₆alkyl-O—, heteroaryl-O— or aryl-, said aryl-C₀-C₆alkyl-O—, heteroaryl-C₀-C₆alkyl-O—, heteroaryl-O— or aryl-groups being further optionally substituted.

32. The compound according to claim 29 of the formula (II) 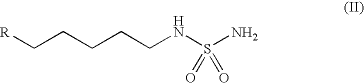
or an N-oxide, hydrate, solvate, pharmaceutically acceptable salt, prodrug or complex thereof, wherein R is selected from the group consisting of:

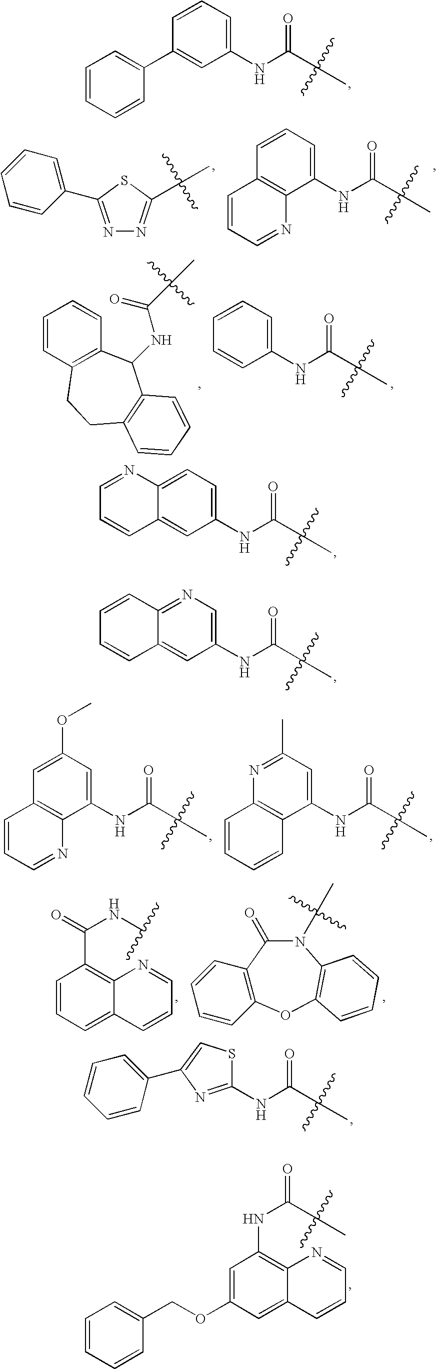

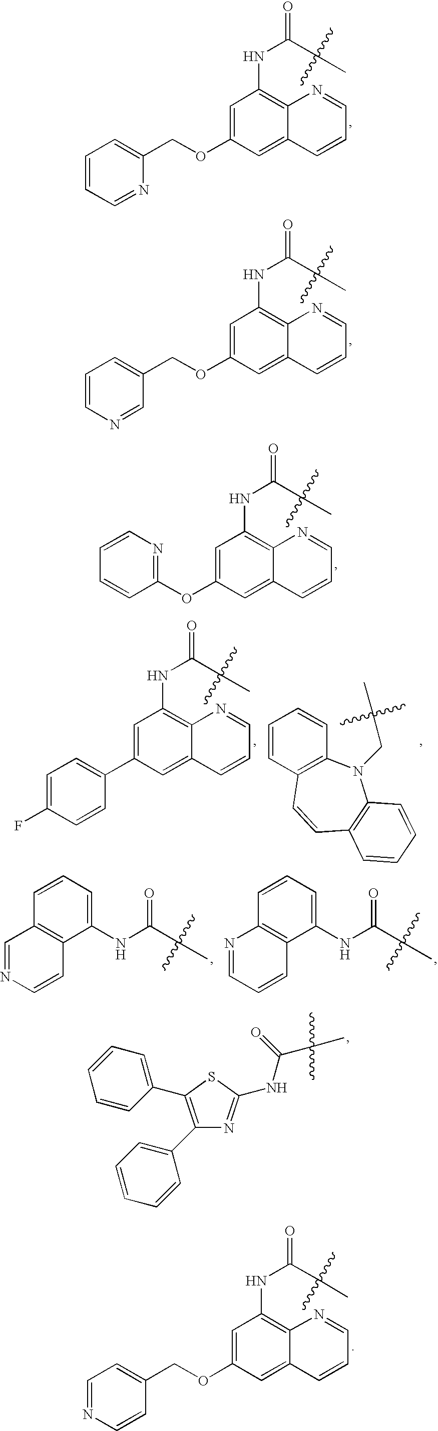

33. The compound according to claim 1, wherein
W is nitrogen or oxygen;
M is nitrogen;
R^{a}, R^{b} and R^{c} are —H;
Z is —C₁-C₈ alkyl- or —C₁-C₈ alkyl-C(O)—;
L is —C₀-C₆ alkyl-N(R³)C(O)—C₀-C₃ alkyl; and
Y is aryl, heteroaryl, heteroaryl-aryl or aryl-heteroaryl wherein the alkyl, aryl and heteroaryl groups are optionally substituted.

34. The compound according to claim 33, wherein the alkyl, aryl and heteroaryl groups are optionally substituted with a substituent selected from the group consisting of alkoxy, alkyl, aryl, —O-alkyl-heteroaryl and —O-alkyl-aryl.

35. The compound according to claim 33 of the formula 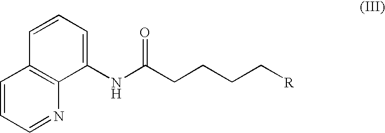
or an N-oxide, hydrate, solvate, pharmaceutically acceptable salt, prodrug or complex thereof, wherein R is selected from the group consisting of:

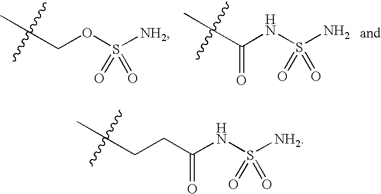

36. The compound according to claim 1, wherein
W and M are nitrogen;
R^{a}, R^{b} and R^{c} are —H;
R³ is —H or C₁-C₆alkyl;
Z is optionally substituted —C₁-C₈ alkyl-;
L is selected from the group consisting of
—C₀-C₆ alkyl-C(O)—N(R³)—C₀-C₃ alkyl-, —C₀-C₆alkyl-N(R³)—C(O)—N(R³)—C₀-C₃alkyl-, or —C₀-C₆alkyl-O—C₀-C₃alkyl-C(O)—N(R³)—C₀-C₃alkyl-, wherein when a C₀-C₃alkyl is C₁-C₃alkyl, the C₁-C₃ alkyl is optionally substituted with —C(O)—N(R³)—C₀-C₃ alkyl-Y, aryl, heteroaryl, -heteroaryl-aryl, -aryl-heteroaryl, -aryl-aryl, heteroaryl-heteroaryl, —N(R³)(R^{3a}) or —N(R³)—Y, wherein each heteroaryl or aryl moeity is optionally substituted;
—C₀-C₆ alkyl-heteroalkyl-C₀-C₆ alkyl-C(O)—N(R³)—C₀-C₃ alkyl-, wherein when the C₀-C₃alkyl is C₁-C₃alkyl, the C₁-C₃ alkyl is optionally substituted with heteroaryl, —N(R³)(R^{3a}) or —N(R³)—Y; and
—C₀-C₆ alkyl-N(R³)C(O)—C₀-C₃ alkyl-, wherein when the C₀-C₃alkyl is C₁-C₃alkyl, the C₁-C₃ alkyl is optionally substituted with —N(R³)—C(O)—O—C₀-C₃alkyl-Y, —NH₂, —NH—S(O)₂—Y, —NH—C(O)—NH—C₀-C₃alkyl-Y, —NH-heteroaryl-aryl or —N(R³)C(O)—C₀-C₃ alkyl-Y, —N(R³)(R^{3a}) or —N(R³)—Y; and
Y is selected from the group consisting of H, cycloalkyl, aryl, heterocyclyl, heteroaryl-aryl, aryl-heteroaryl, heterocyclyl-O—, aryl-N(R³)—C(O)-heteroaryl, heteroaryl-N(R³)—C(O)-heteroaryl, aryl-N(R³)—C(O)-aryl, heterocyclyl-C₀-C₆alkyl-N(R³)—C(O)-heteroaryl and B²—B¹—N(R³)—C(O)—C₁-C₇ alkyl-, wherein the alkyl, cycloalkyl, aryl, heteroaryl and heterocyclyl moieties are optionally substituted and the C₁-C₇ alkyl is optionally substituted with —NR³—B³ and the amine of B³ is connected with the acid of B² to form a peptide bond, and wherein when Y is B²—B¹—N(R³)—C(O)—C₁-C₇ alkyl-, then Z and L are covalent bonds; wherein
when any of B¹, B² and B³ are attached together, they are attached by a peptide bond, and B¹, B² and B³ are independently selected from the group consisting of D-Pro, L-ile and D-Phe-4-CF₃.

37. The compound according to claim 36, wherein
Y is selected from the group consisting of H, cycloalkyl, aryl, heteroaryl, heteroaryl-aryl, aryl-heteroaryl, heterocyclyl-O—, aryl-N(R³)—C(O)-heteroaryl, heteroaryl-N(R³)—C(O)-heteroaryl, aryl-N(R³)—C(O)-aryl, heterocyclyl-C₀-C₆alkyl-N(R³)—C(O)-heteroaryl and B²—B¹—N(R³)—C(O)—C₁-C₇ alkyl-, wherein the cycloalkyl, aryl, heteroaryl, heterocyclyl and alkyl moieties are optionally substituted with one, two or three (preferably one or two, more preferably one) substituents selected from the group consisting of halo, alkoxy, optionally substituted C₁-C₆alkyl, alkoxycarbonyl-, —OH, —CN, —C(O)—OH, optionally substituted aryl, optionally substituted -alkylaryl, optionally substituted heteroaryl, optionally substituted —O—C₁-C₆alkyl-aryl, optionally substituted —C(O)—O—C₁-C₆alkyl, —NH₂, optionally substituted -aryl-heterocyclyl and optionally substituted fused heterocyle, and the C₁-C₇ alkyl is optionally substituted with —NR³—B³ and the amine of B³ is connected with the acid of B² to form a peptide bond, and wherein when Y is B²—B¹—N(R³)—C(O)—C₁-C₇ alkyl-, then Z and L are covalent bonds; wherein
when any of B¹, B² and B³ are attached together, they are attached by a peptide bond, and B¹, B² and B³ are independently selected from the group consisting of D-Pro, L-ile and D-Phe-4-CF₃.

38. The compound according to claim 36, wherein
L is —C₀-C₆ alkyl-N(R³)C(O)—C₀-C₃ alkyl-, wherein when the C₀-C₃alkyl is C₁-C₃alkyl, the C₁-C₃ alkyl is optionally substituted with —N(R³)—C(O)—O—C₀-C₃alkyl-Y, —NH₂, —NH—S(O)₂—Y, —NH—C(O)—NH—C₀-C₃alkyl-Y, —NH-heteroaryl-aryl, —N(R³)C(O)—C₀-C₃ alkyl-Y, —N(R³)(R^{3a}) or —N(R³)—Y.

39. The compound according to claim 36, wherein
L is —C₀-C₆ alkyl-N(R³)C(O)—C₀-C₃ alkyl-, wherein when the C₀-C₃alkyl is C₁-C₃alkyl, the C₁-C₃ alkyl is optionally substituted with —N(R³)—C(O)—O—C₀-C₃alkyl-Y, —NH₂, —NH—S(O)₂—Y, —NH—C(O)—NH—C₀-C₃alkyl-Y, —NH-heteroaryl-aryl, —N(R³)C(O)—C₀-C₃ alkyl-Y, —N(R³)(R^{3a}) or —N(R³)—Y; and
Y is selected from the group consisting of H, cycloalkyl, aryl, heteroaryl, heteroaryl-aryl, aryl-heteroaryl, heterocyclyl-O—, aryl-N(R³)—C(O)-heteroaryl, heteroaryl-N(R³)—C(O)-heteroaryl, aryl-N(R³)—C(O)-aryl, heterocyclyl-C₀-C₆alkyl-N(R³)—C(O)-heteroaryl and B²—B¹—N(R³)—C(O)—C₁-C₇ alkyl-, wherein the cycloalkyl, aryl, heteroaryl, heterocyclyl and alkyl groups are optionally substituted with one, two or three (preferably one or two, more preferably one) substituents selected from the group consisting of halo, alkoxy, optionally substituted C₁-C₆alkyl, alkoxycarbonyl-, —OH, —CN, —C(O)—OH, optionally substituted aryl, optionally substituted -alkylaryl, optionally substituted heteroaryl, optionally substituted —O—C₁-C₆alkyl-aryl, optionally substituted —C(O)—O—C₁-C₆alkyl, —NH₂, optionally substituted -aryl-heterocyclyl and optionally substituted fused heterocyle, and the C₁-C₇ alkyl is optionally substituted with —NR³—B³ and the amine of B³ is connected with the acid of B² to form a peptide bond, and wherein when Y is B 2-B¹—N(R³)—C(O)—C₁-C₇ alkyl-, then Z and L are covalent bonds; wherein
when any of B¹, B² and B³ are attached together, they are attached by a peptide bond, and B¹, B² and B³ are independently selected from the group consisting of D-Pro, L-ile and D-Phe-4-CF₃.

40. The compound according to claim 36, wherein
W and M are nitrogen;
R^{a}, R^{b} and R^{c} are —H;
R³ is —H;
Z is optionally substituted —C₁-C₈ alkyl-;
L is selected from the group consisting of
—C₀-C₆ alkyl-C(O)—N(R³)—C₀-C₃ alkyl-, wherein when the C₀-C₃alkyl is C₁-C₃alkyl, the C₁-C₃ alkyl is optionally substituted with —C(O)—N(R³)—C₀-C₃ alkyl-Y, -heteroaryl-aryl, heteroaryl, —N(R³)(R^{3a}) or —N(R³)—Y;
—C₀-C₆ alkyl-heteroalkyl-C₀-C₆ alkyl-C(O)—N(R³)—C₀-C₃ alkyl-, wherein when the C₀-C₃alkyl is C₁-C₃alkyl, the C₁-C₃ alkyl is optionally substituted with heteroaryl, —N(R³)(R^{3a}) or —N(R³)—Y; and
—C₀-C₆ alkyl-N(R³)C(O)—C₀-C₃ alkyl-, wherein when the C₀-C₃alkyl is C₁-C₃alkyl, the C₁-C₃ alkyl is optionally substituted with —N(R³)C(O)—C₀-C₃ alkyl-Y, —N(R³)(R^{3a}) or —N(R³)—Y; and
Y is selected from the group consisting of aryl, heteroaryl, heteroaryl-aryl, aryl-heteroaryl and B²—B¹—N(R³)—C(O)—C₁-C₇ alkyl-, wherein the aryl and heteroaryl are optionally substituted, and the C₁-C₇ alkyl is optionally substituted with —NR³—B³ and the amine of B³ is connected with the acid of B² to form a peptide bond, and wherein when Y is B²—B¹—N(R³)—C(O)—C₁-C₇ alkyl-, then Z and L are covalent bonds; wherein
when any of B¹, B² and B³ are attached together, they are attached by a peptide bond, and
B¹, B² and B³ are independently selected from the group consisting of D-Pro, L-ile and D-Phe-4-CF₃.

41. The compound according to claim 40, wherein
Y is selected from the group consisting of aryl, heteroaryl, heteroaryl-aryl, aryl-heteroaryl and B²—B¹—N(R³)—C(O)—C₁-C₇ alkyl-, wherein the aryl and heteroaryl groups are optionally substituted with one, two or three (preferably one or two, more preferably one) substituents selected from the group consisting of halo, alkoxy, optionally substituted C₁-C₆alkyl, alkoxycarbonyl-, —OH, —CN, —C(O)—OH, optionally substituted aryl, optionally substituted -alkylaryl, optionally substituted heteroaryl, optionally substituted —O—C₁-C₆alkyl-aryl, optionally substituted —C(O)—O—C₁-C₆alkyl, —NH₂, optionally substituted -aryl-heterocyclyl and optionally substituted fused heterocyle, and the C₁-C₇ alkyl is optionally substituted with —NR³—B³ and the amine of B³ is connected with the acid of B² to form a peptide bond, and wherein when Y is B²—B¹—N(R³)—C(O)—C₁-C₇ alkyl-, then Z and L are covalent bonds; wherein
when any of B¹, B² and B³ are attached together, they are attached by a peptide bond, and B¹, B² and B³ are independently selected from the group consisting of D-Pro, L-ile and D-Phe-4-CF₃.

42. The compound according to claim 40, wherein
L is selected from the group consisting of
—C₀-C₆ alkyl-N(R³)C(O)—C₀-C₃ alkyl-, wherein when the C₀-C₃alkyl is C₁-C₃alkyl, the C₁-C₃ alkyl is optionally substituted with —N(R³)C(O)—C₀-C₃ alkyl-Y, —N(R³)(R^{3a}) or —N(R³)—Y; and
Y is selected from the group consisting of aryl, heteroaryl, heteroaryl-aryl, aryl-heteroaryl and B²—B¹—N(R³)—C(O)—C₁-C₇ alkyl-, wherein the aryl and heteroaryl groups are optionally substituted, and the C₁-C₇ alkyl is optionally substituted with —NR³—B³ and the amine of B³ is connected with the acid of B² to form a peptide bond, and wherein when Y is B²—B¹—N(R³)—C(O)—C₁-C₇ alkyl-, then Z and L are covalent bonds; wherein
when any of B¹, B² and B³ are attached together, they are attached by a peptide bond, and
B¹, B² and B³ are independently selected from the group consisting of D-Pro, L-ile and D-Phe-4-CF₃.

43. The compound according to claim 40, wherein
L is —C₀-C₆ alkyl-N(R³)C(O)—C₀-C₃ alkyl-, wherein when the C₀-C₃alkyl is C₁-C₃alkyl, the C₁-C₃ alkyl is optionally substituted with —N(R³)C(O)—C₀-C₃ alkyl-Y, —N(R³)(R^{3a}) or —N(R³)—Y; and
Y is selected from the group consisting of aryl, heteroaryl, heteroaryl-aryl, aryl-heteroaryl and B²—B¹—N(R³)—C(O)—C₁-C₇ alkyl-, wherein the aryl and heteroaryl groups are optionally substituted with one, two or three (preferably one or two, more preferably one) substituents selected from the group consisting of halo, alkoxy, optionally substituted C₁-C₆alkyl, alkoxycarbonyl-, —OH, —CN, —C(O)—OH, optionally substituted aryl, optionally substituted -alkylaryl, optionally substituted heteroaryl, optionally substituted —O—C₁-C₆alkyl-aryl, optionally substituted —C(O)—O—C₁-C₆alkyl, —NH₂, optionally substituted -aryl-heterocyclyl and optionally substituted fused heterocyle, and the C₁-C₇ alkyl is optionally substituted with —NR³—B³ and the amine of B³ is connected with the acid of B² to form a peptide bond, and wherein when Y is B²—B¹—N(R³)—C(O)—C₁-C₇ alkyl-, then Z and L are covalent bonds; wherein
when any of B¹, B² and B³ are attached together, they are attached by a peptide bond, and
B¹, B² and B³ are independently selected from the group consisting of D-Pro, L-ile and D-Phe-4-CF₃.

44. The compound according to claim 36, wherein
L is —C₀-C₆ alkyl-C(O)—N(R³)—C₀-C₃ alkyl-, wherein when the C₀-C₃alkyl is C₁-C₃alkyl, the C₁-C₃ alkyl is optionally substituted with aryl, heteroaryl, -heteroaryl-aryl, -aryl-heteroaryl, -aryl-aryl or heteroaryl-heteroaryl, wherein each heteroaryl or aryl moeity is optionally substituted; and
Y is aryl or heteroaryl, each of which is optionally substituted.

45. The compound according to claim 44 selected from the group consisting of

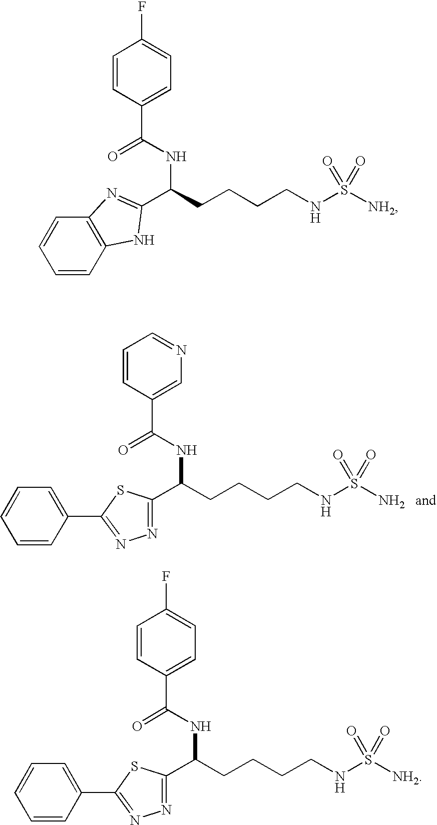

46. The compound according to claim 36, wherein
L is —C₀-C₆alkyl-O—C₀-C₁alkyl-C(O)—N(R³)—C₀-C₃alkyl-, wherein when the C₀-C₃alkyl is C₁-C₃alkyl, the C₁-C₃ alkyl is optionally substituted with -heteroaryl-aryl, -heteroaryl-heteroaryl, heteroaryl, -heteroaryl-heterocylcyl, wherein each heteroaryl and aryl moeity is optionally substituted; and
Y is optionally substituted aryl.

47. The compound according to claim 46, wherein when the C₀-C₃alkyl is C₁-C₃alkyl, the C₁-C₃ alkyl is optionally substituted with -heteroaryl-aryl, -heteroaryl-heteroaryl, heteroaryl, -heteroaryl-heterocylcyl, wherein each heteroaryl and aryl moeity is further optionally substituted with 1 to 3 of optionally substituted aryl, alkoxy, —N(alkyl)₂, halogen, alkyl, fused heterocyclyl, —CF₃, optionally substituted heterocyclyl, —O—C₁-C₆alkyl-N(alkyl)₂, —O—C₁-C₆alkyl-NH2 and —NH-aryl.

48. The compound of claim 46 selected from the group consisting of

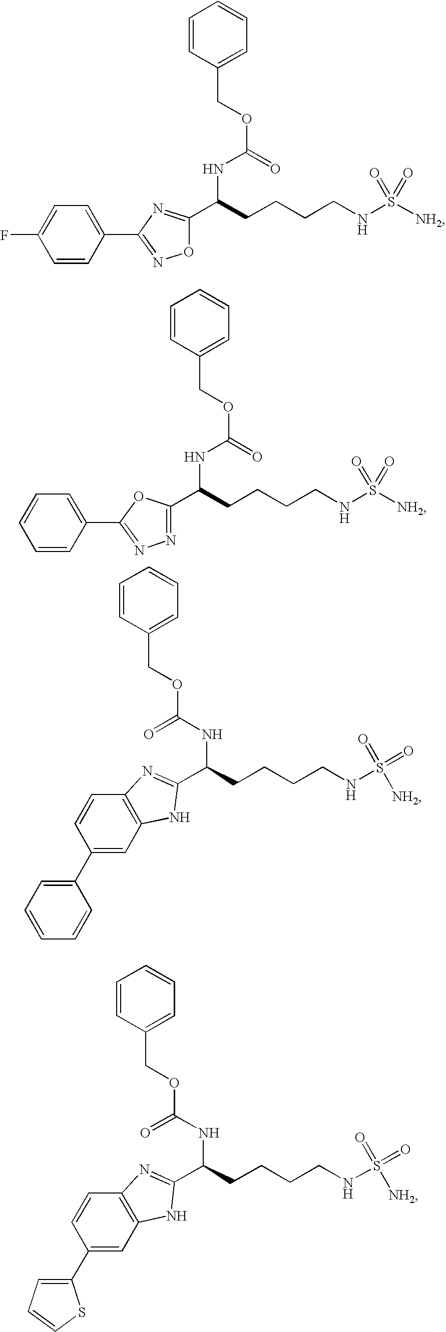

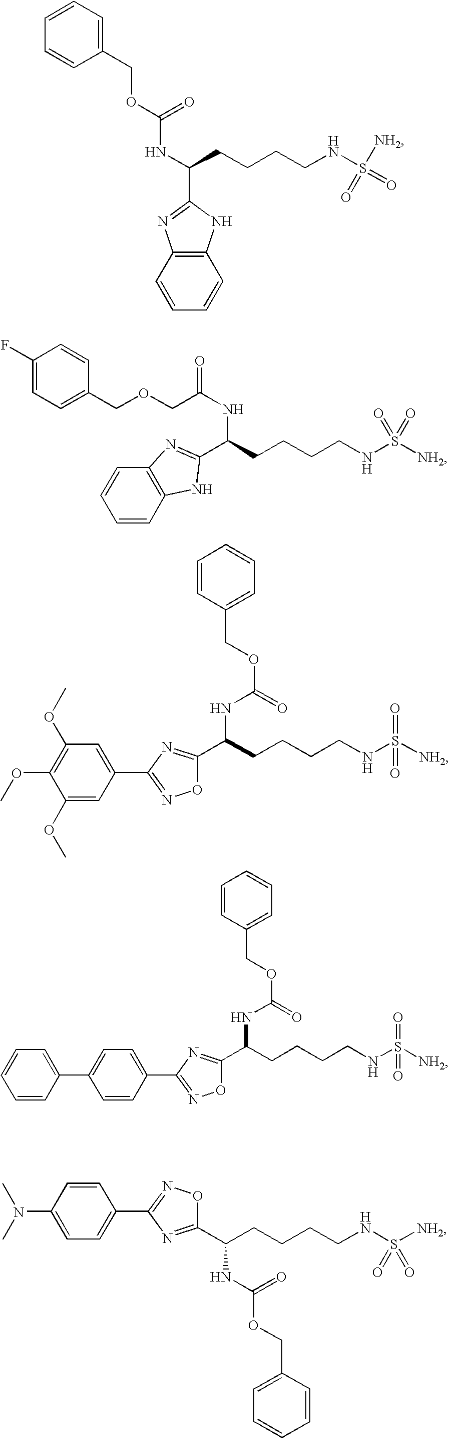

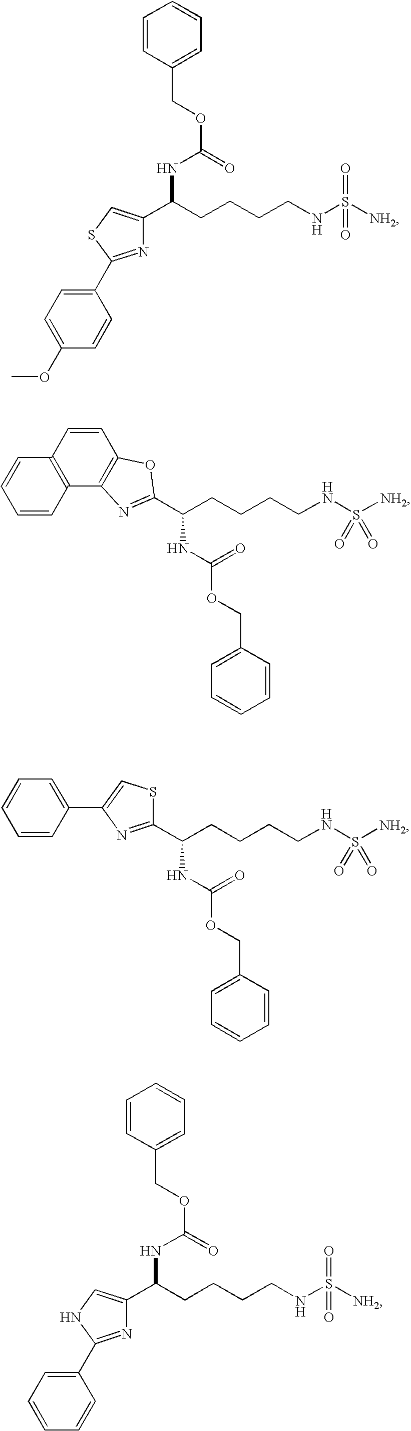

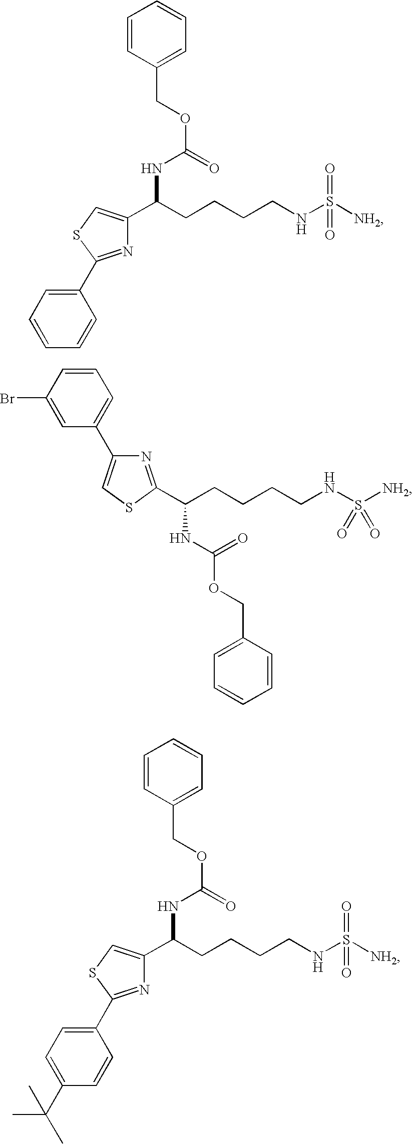

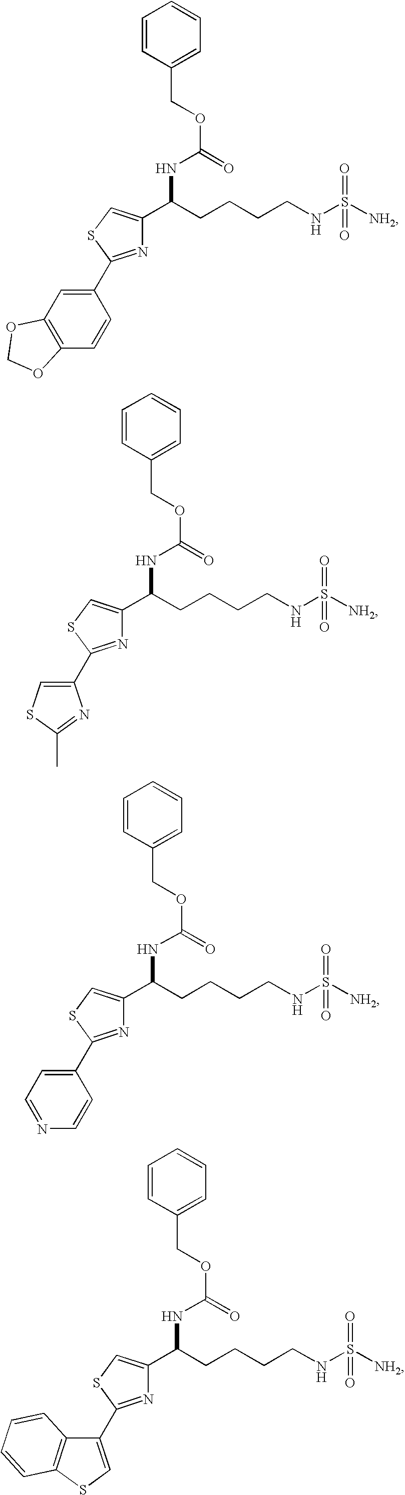

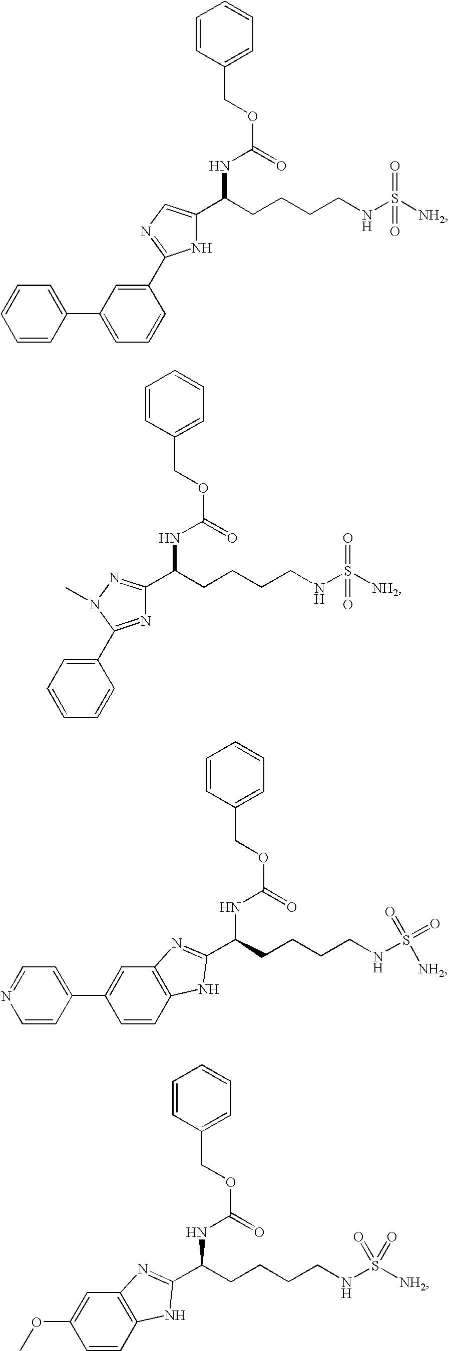

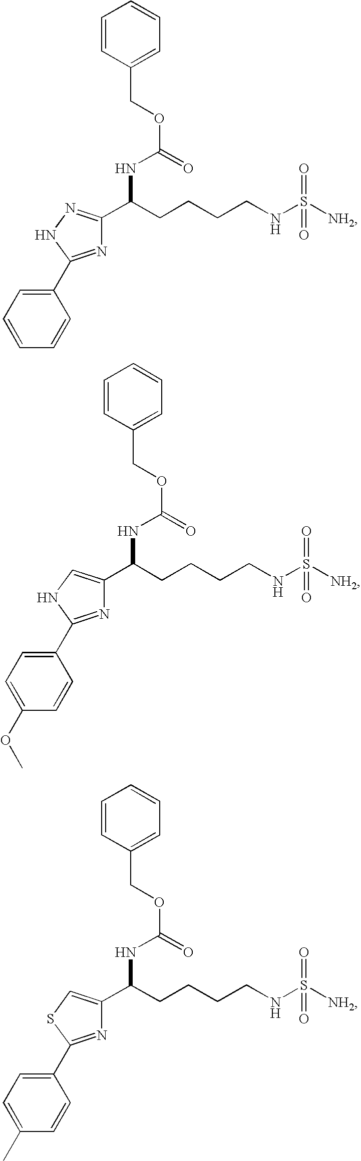

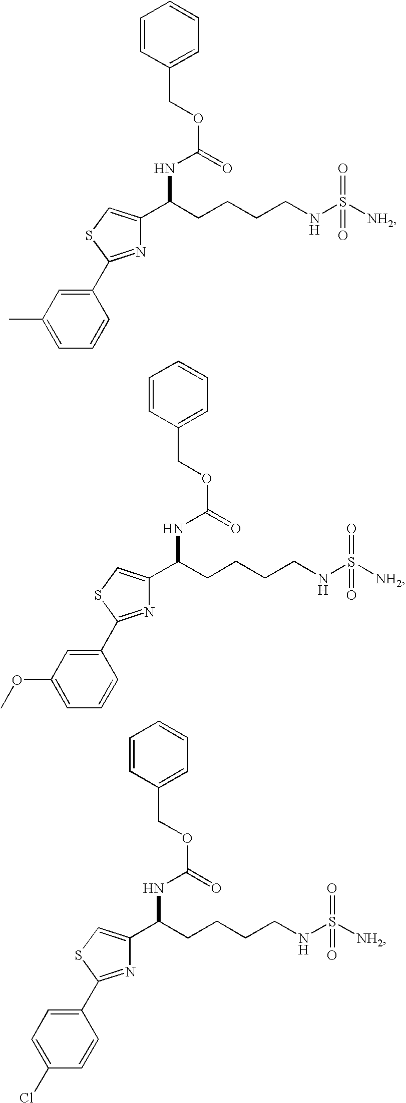

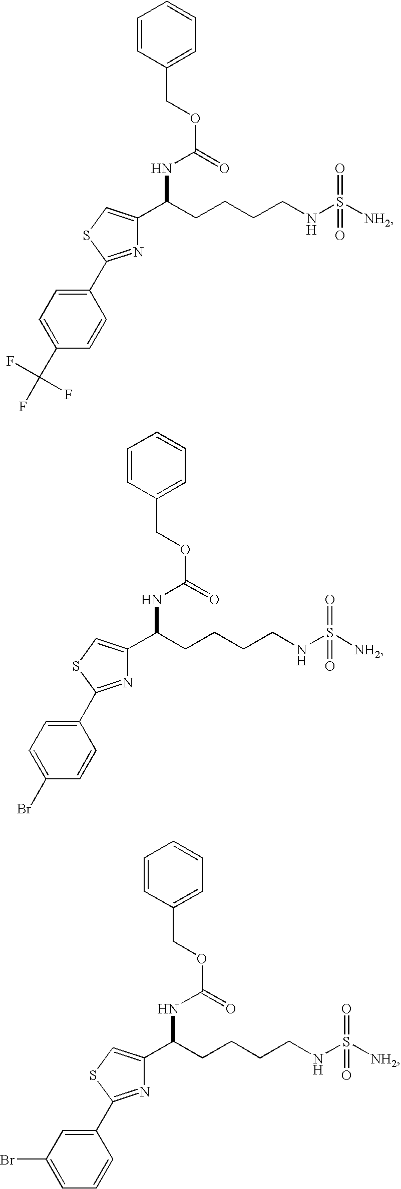

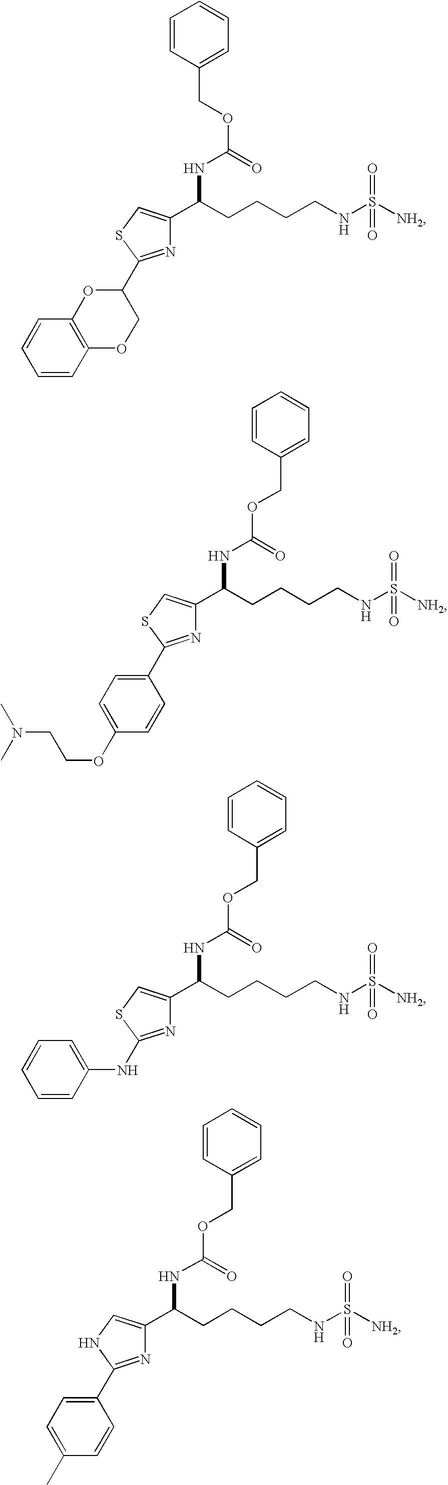

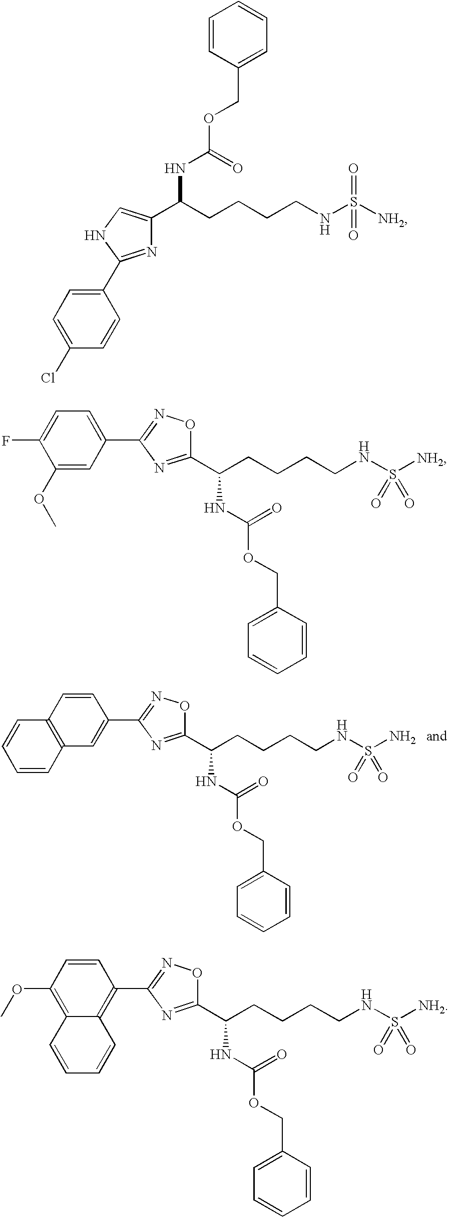

49. The compound according to claim 36, wherein
L is —C₀-C₆ alkyl-C(O)—N(R³)—C₀-C₃ alkyl-, wherein when the C₀-C₃alkyl is C₁-C₃alkyl, the C₁-C₃ alkyl is optionally substituted with —C(O)—N(R³)—C₀-C₃alkyl-heteroaryl, —C(O)—N(R³)—C₀-C₃alkyl-aryl, wherein each heteroaryl or aryl moeity is optionally substituted; and
Y is H, optionally substituted aryl, optionally substituted heterocyclyl.

50. The compound according to claim 49, wherein Y is optionally substituted heteroaryl.

51. The compound according to claim 49, wherein Y is optionally substituted aryl, optionally substituted heteroaryl, wherein each heteroaryl or aryl moeity is optionally substituted with 1 or 2 independently selected halogen, alkyl or alkoxy.

52. The compound according to claim 49 selected from the group consisting of

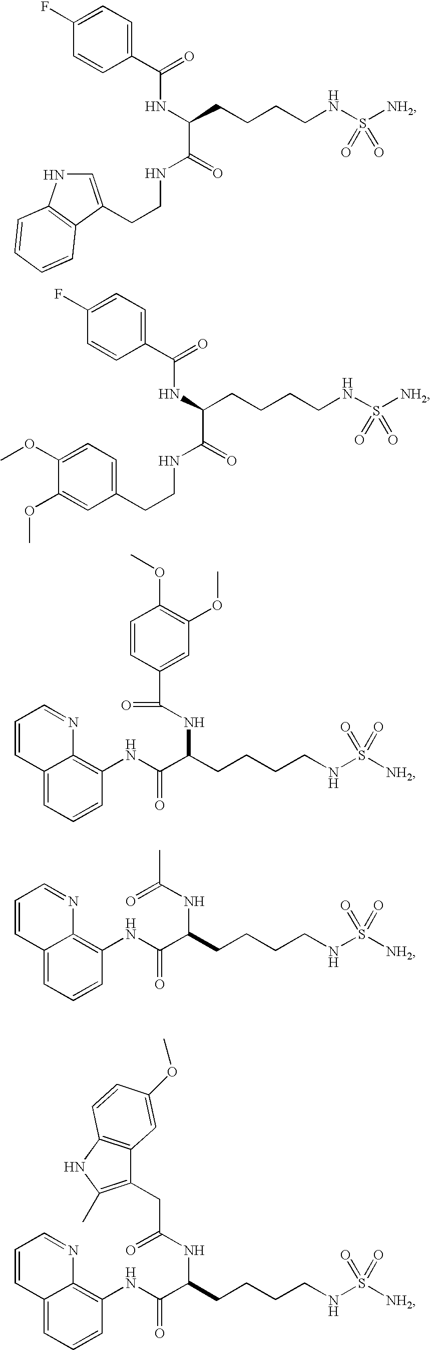

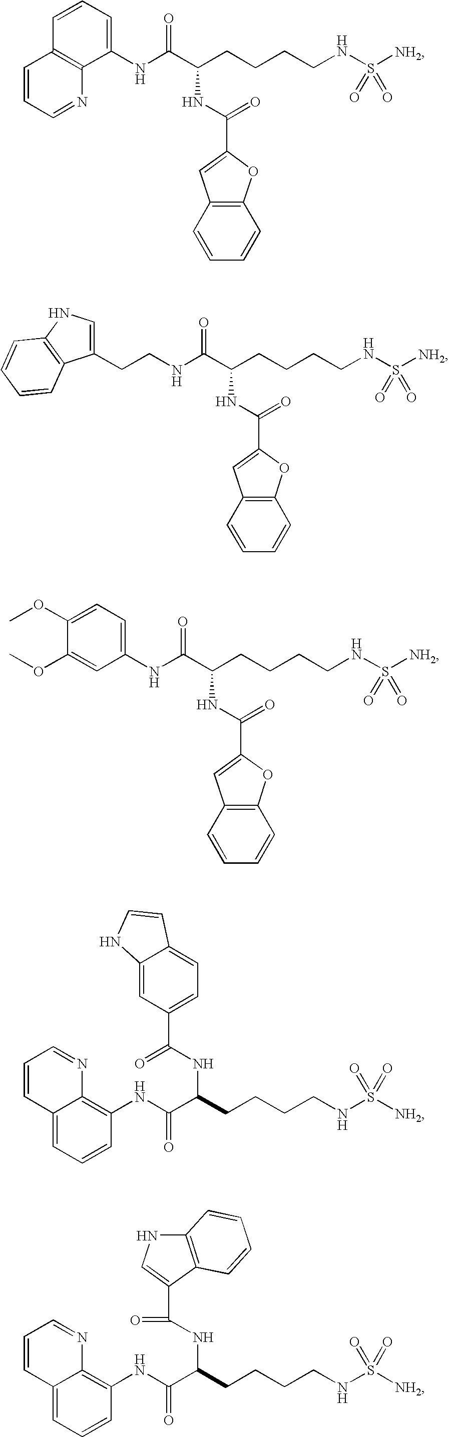

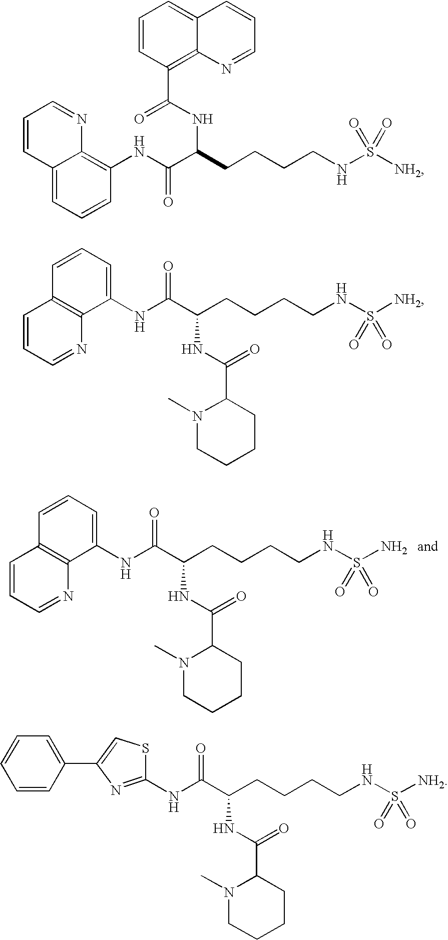

53. The compound according to claim 36, wherein
L is —C₀-C₆alkyl-O—C₀-C₁alkyl-C(O)—N(R³)—C₀-C₃alkyl-, wherein when the C₀-C₃alkyl is C₁-C₃alkyl, the C₁-C₃ alkyl is optionally substituted with —C(O)—N(R³)—C₀-C₃ alkyl-heterocyclyl or —C(O)—N(R³)—C₀-C₃ alkyl-aryl, wherein each heterocyclyl or aryl moeity is optionally substituted; and
Y is optionally substituted aryl, or optionally substituted heteroaryl.

54. The compound according to claim 53, wherein Y is optionally substituted aryl.

55. The compound according to claim 53, wherein —C(O)—N(R³)—C₀-C₃ alkyl-heterocyclyl is —C(O)—N(R³)—C₀-C₃ alkyl-heteroaryl.

56. The compound according to claim 53, wherein Y-L- is phenyl-CH₂—O—C(O)—NH—.

57. The compound according to claim 53, wherein
L is —C₀-C₆alkyl-O—C₀-C₁alkyl-C(O)—N(R³)—C₀-C₃alkyl-, wherein when the C₀-C₃alkyl is C₁-C₃alkyl, the C₁-C₃ alkyl is optionally substituted with —C(O)—N(R³)—C₀-C₃ alkyl-heteroaryl or —C(O)—N(R³)—C₀-C₃ alkyl-aryl, wherein each heteroaryl or aryl moeity is optionally substituted with 1 to 3 independent substituents selected from the group consisting of halogen, —OH, —NH₂, alkyl, —C(O)—OH, —C(O)—O-alkyl, —C(O)—NH-optionally substituted aryl, —C(O)—NH-optionally substituted heteroaryl, —C(O)—NH-alkyl-O-alkyl, —C(O)—NH-alkyl-heterocyclyl, -alkyl-optionally substituted aryl, alkoxy, optionally substituted aryl, optionally substituted heteroaryl.

58. The compound according to claim 57, wherein the substituents selected from the group consisting of —C(O)—NH-optionally substituted aryl, —C(O)—NH-optionally substituted heteroaryl, -alkyl-optionally substituted aryl, optionally substituted aryl and optionally substituted heteroaryl, are optionally substituted with 1 or 2 independently selected substituents selected from the group consisting of halogen, alkoxy, alkyl, —O-aryl, —NH—C(O)-alkyl, oxo, —CN, heterocyclyl, —O-halosubstitutedalkyl, —CF₃ and —O-alkyl-O-alkyl.

59. The compound according to claim 53, wherein
L is phenyl-CH₂—O—C(O)—NH—C₁-C₃alkyl-, wherein the C₁-C₃ alkyl is substituted with —C(O)—NH-thiazolyl, wherein the thiazolyl is optionally substituted.

60. The compound according to claim 53, wherein
L is phenyl-CH₂—O—C(O)—NH—C₁-C₃alkyl-, wherein the C₁-C₃ alkyl is substituted with —C(O)—NH-thiazolyl, wherein the thiazolyl is optionally substituted with 1 or 2 independently selected substituents selected from the group consisting of optionally substituted aryl, alkyl, —C(O)—O-alkyl, —C(O)—OH, —C(O)—NH-optionally substituted aryl, —C(O)—NH-optionally substituted heteroaryl, —C(O)—NH-alkyl-O-alkyl, —C(O)—NH-alkyl-heterocyclyl, fused optionally substituted cycloalkyl, fused optionally substituted heterocyclyl and fused optionally substituted aryl.

61. The compound according to claim 53, selected from the group consisting of

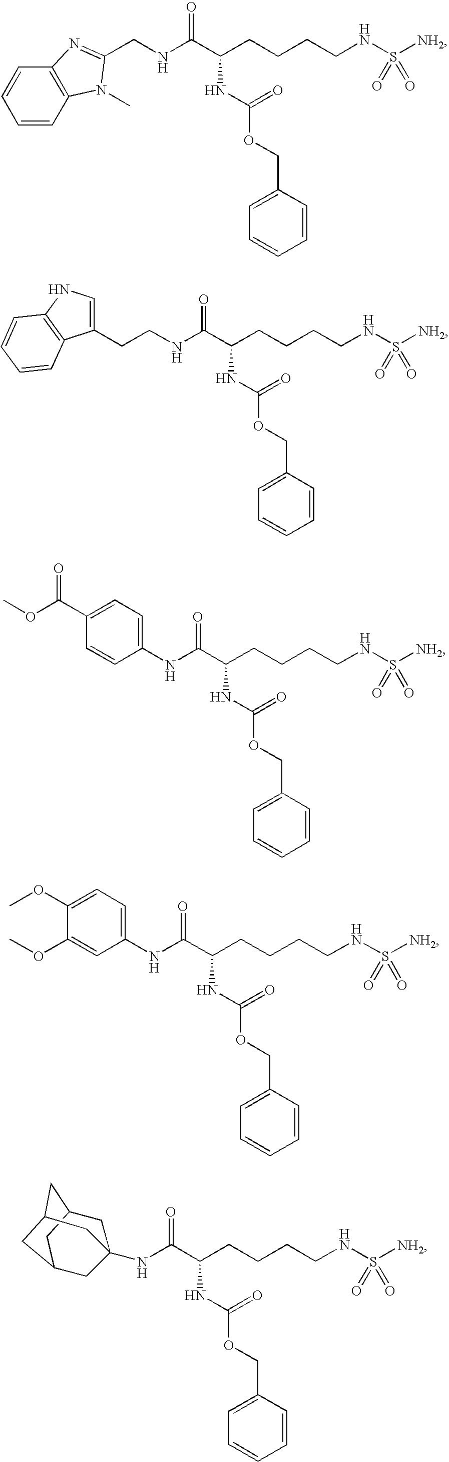

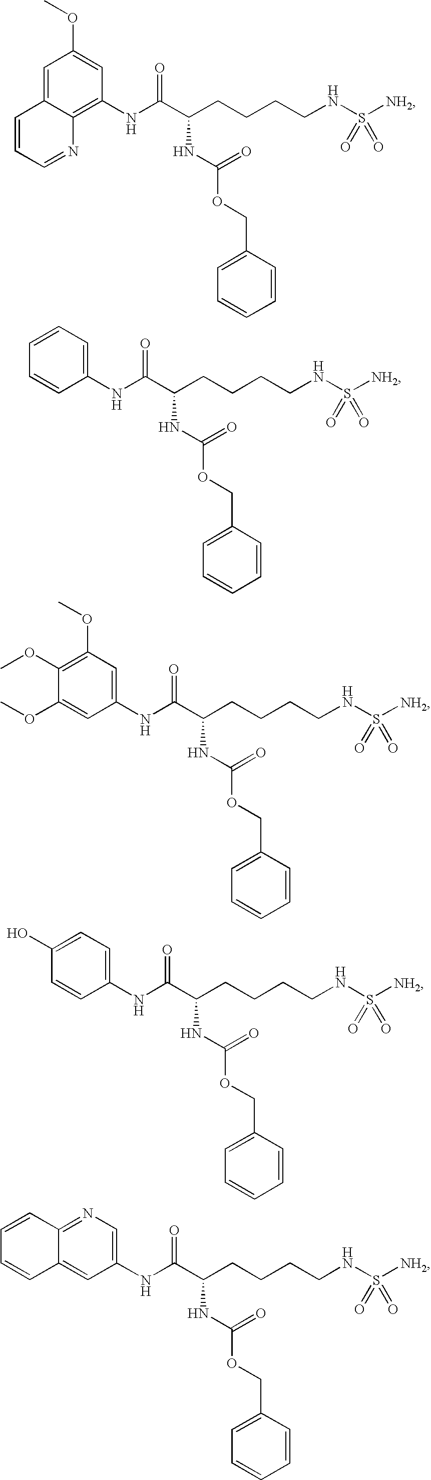

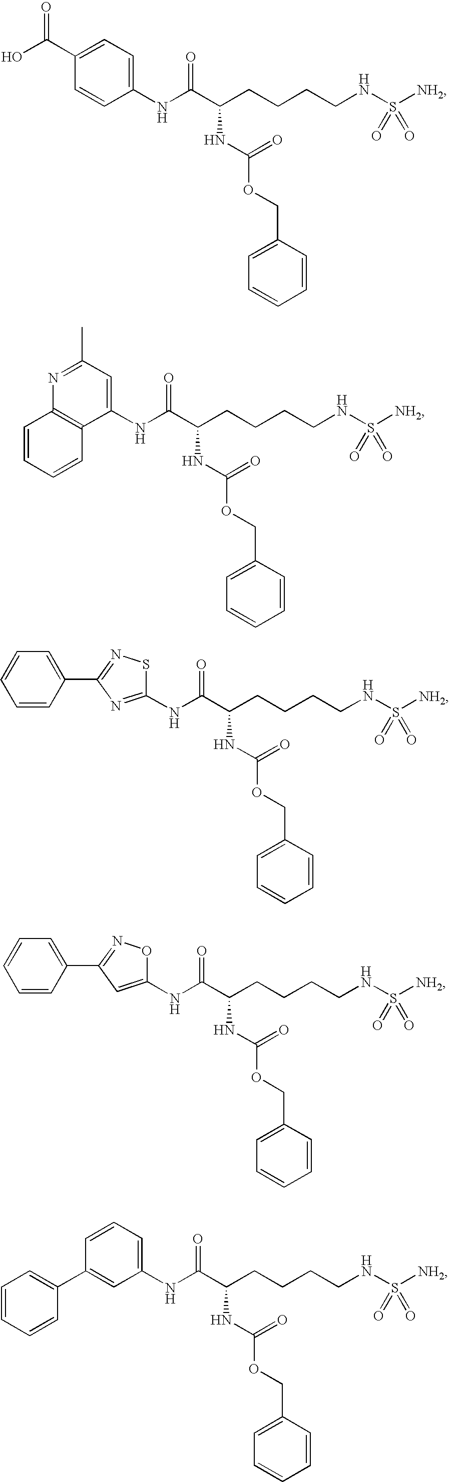

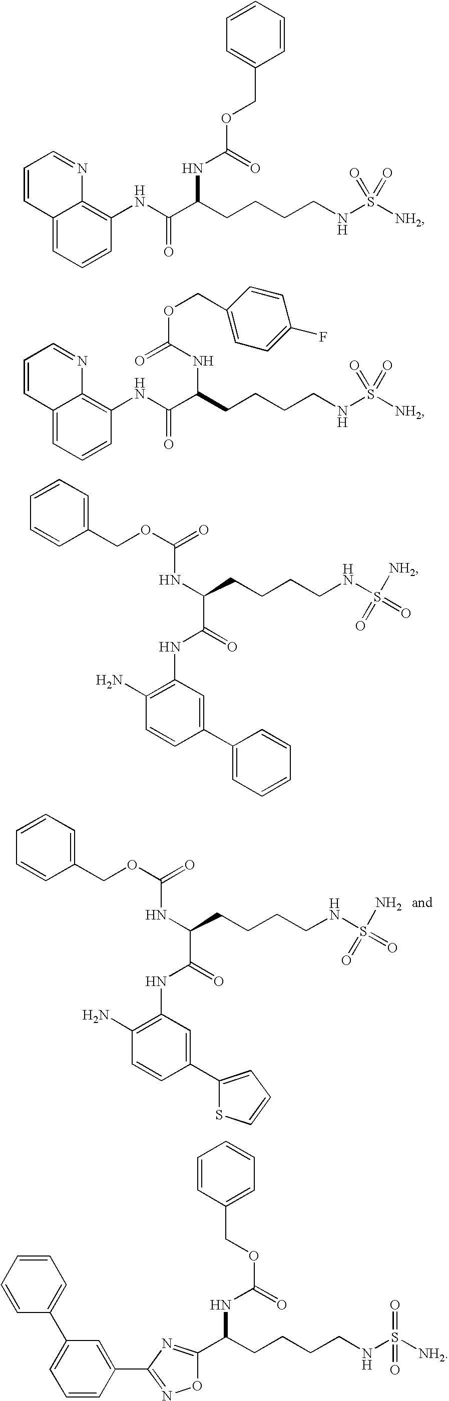

62. The compound according to claim 53, selected from the group consisting of

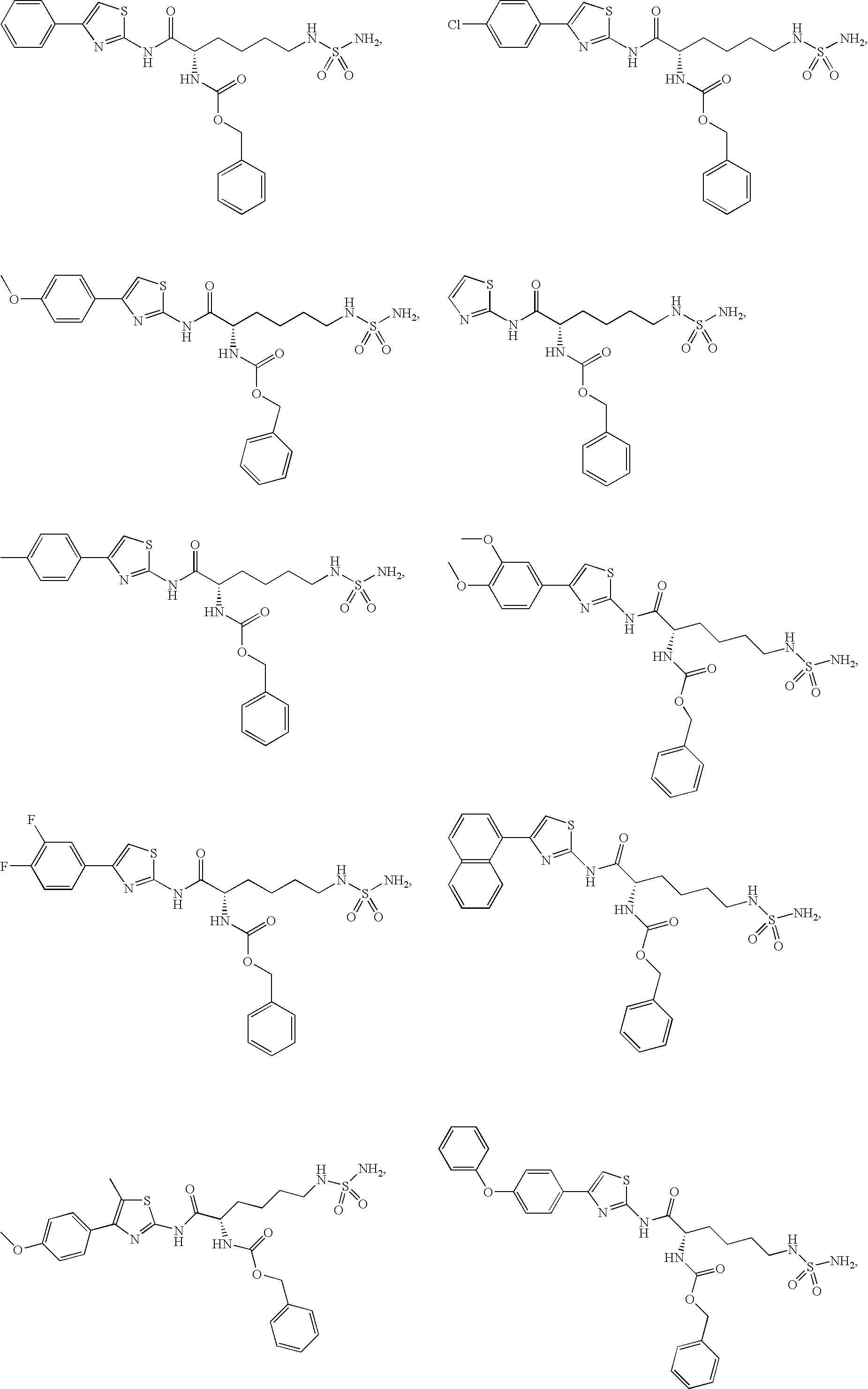

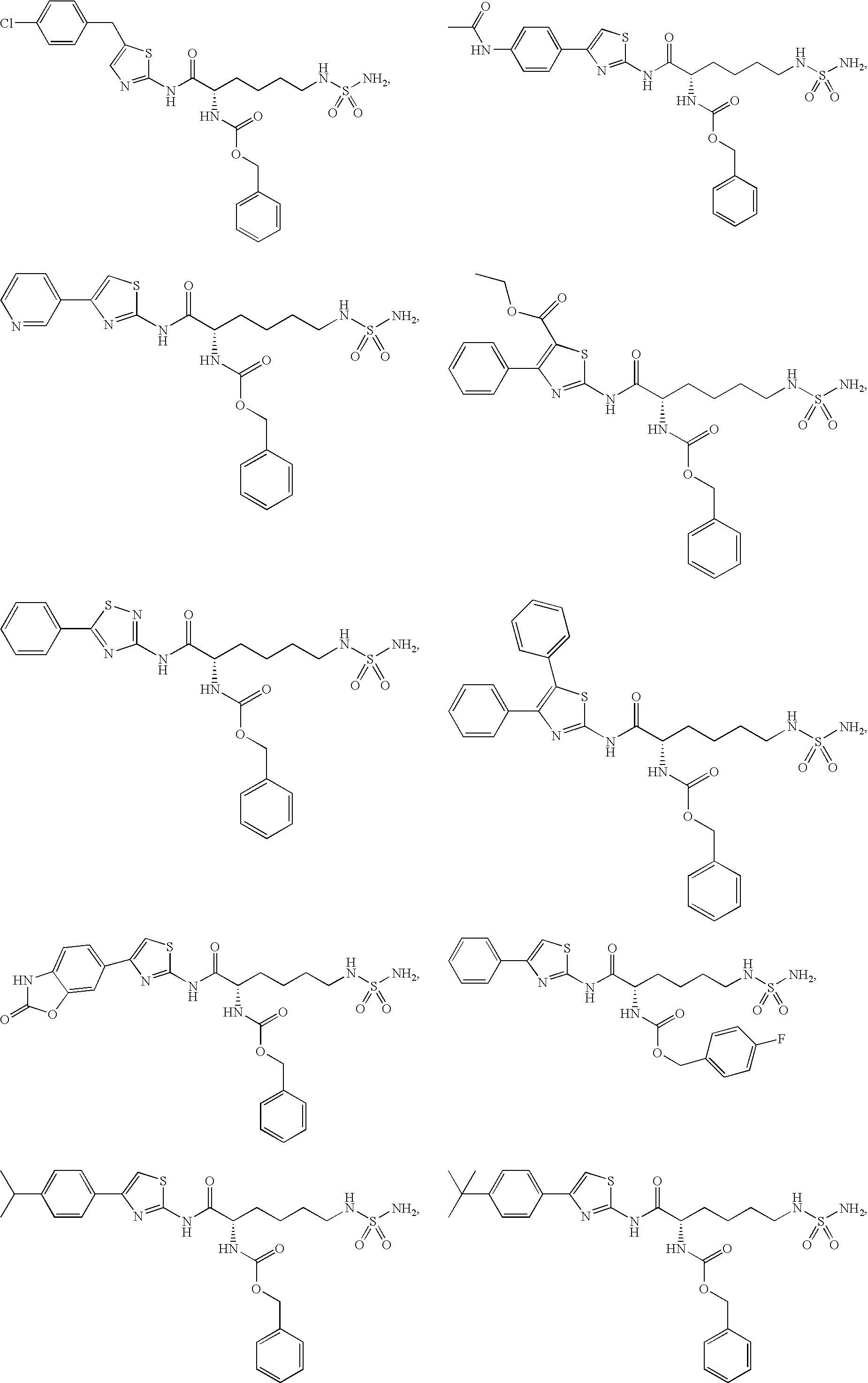

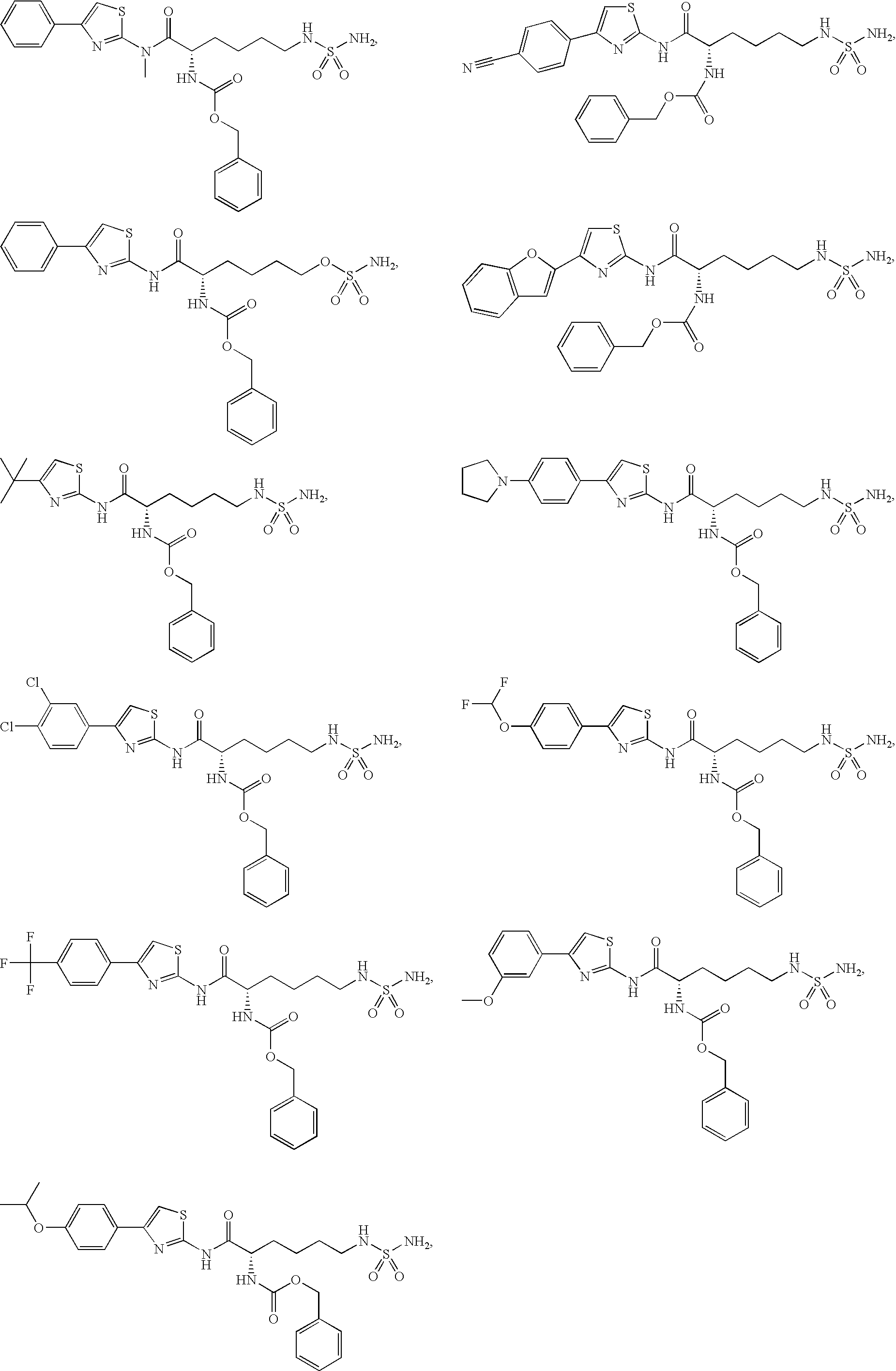

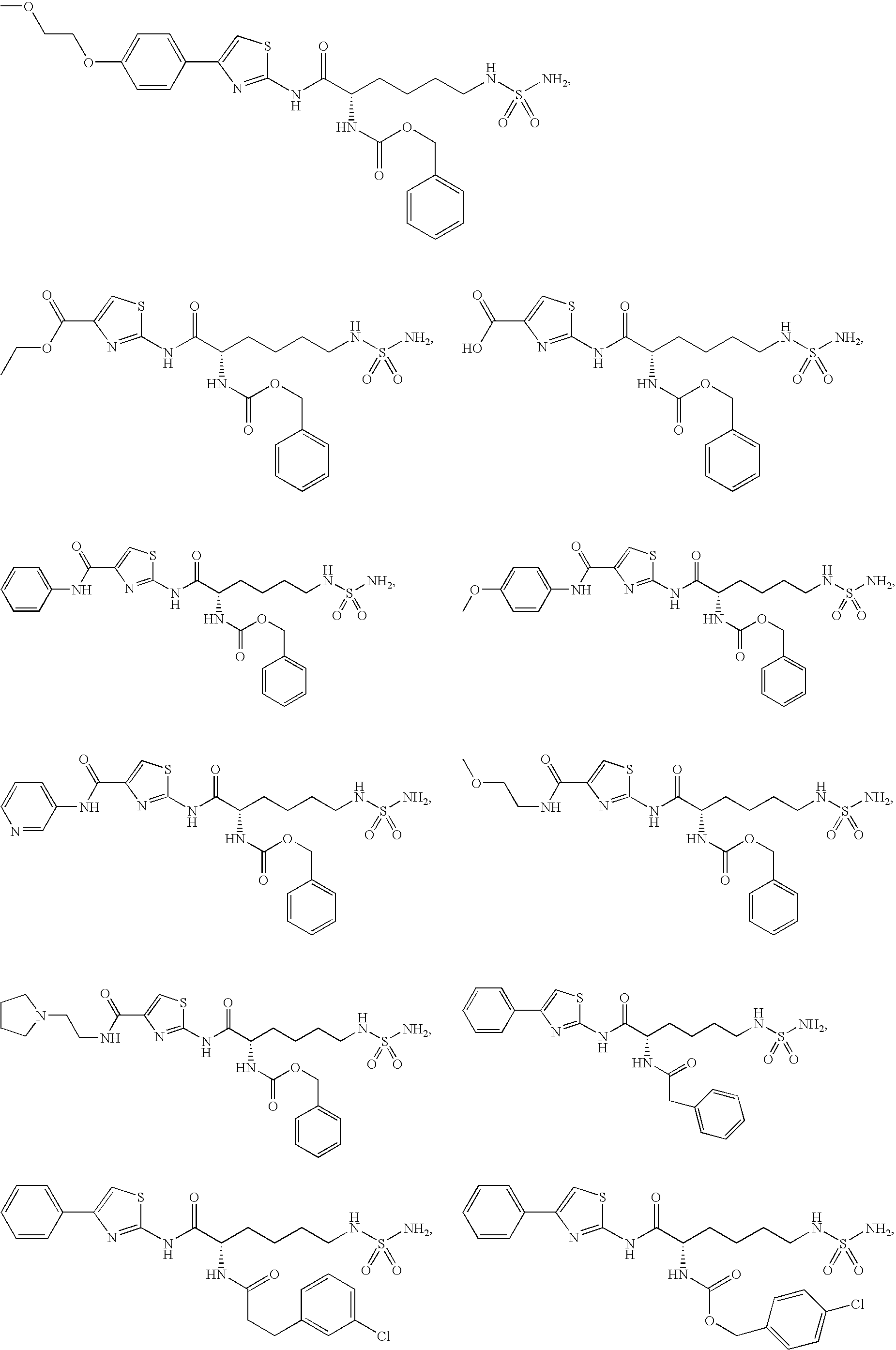

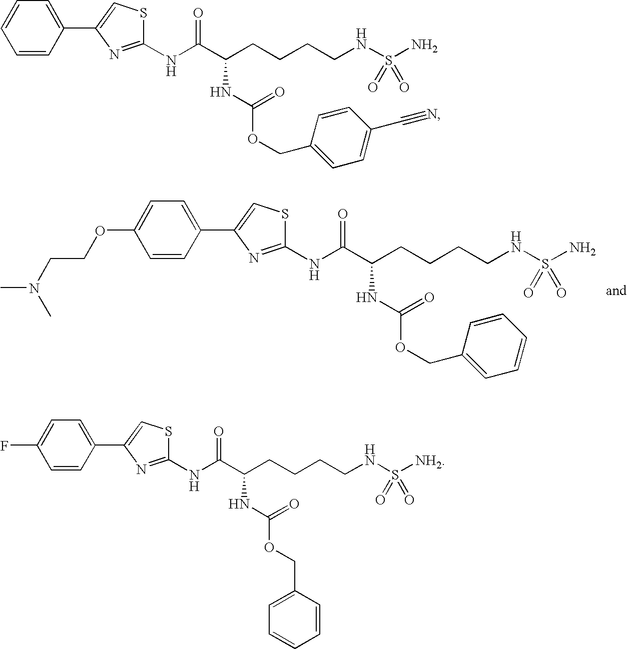

63. The compound according to claim 1, selected from the group consisting of

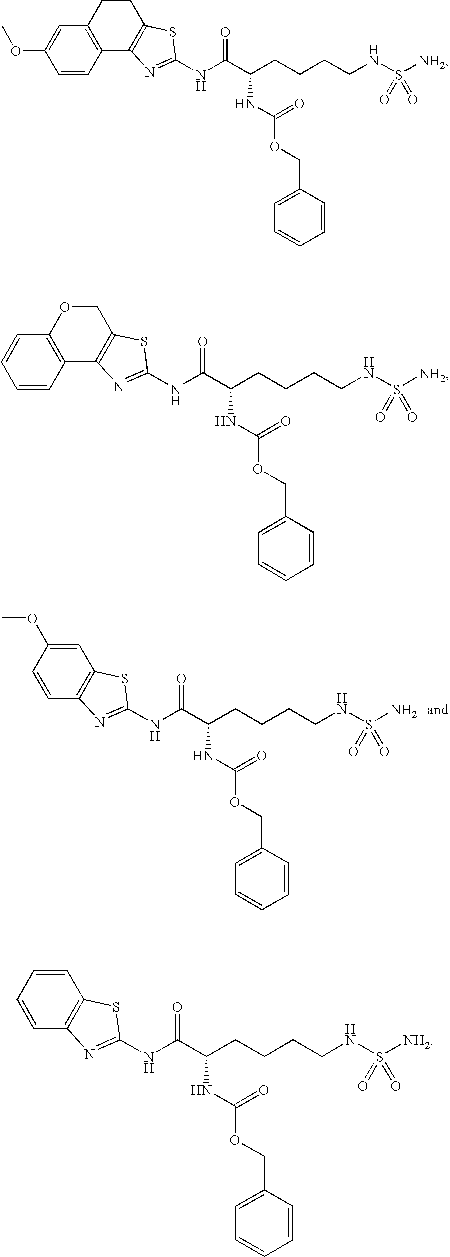

64. The compound according to claim 1, selected from the group consisting of

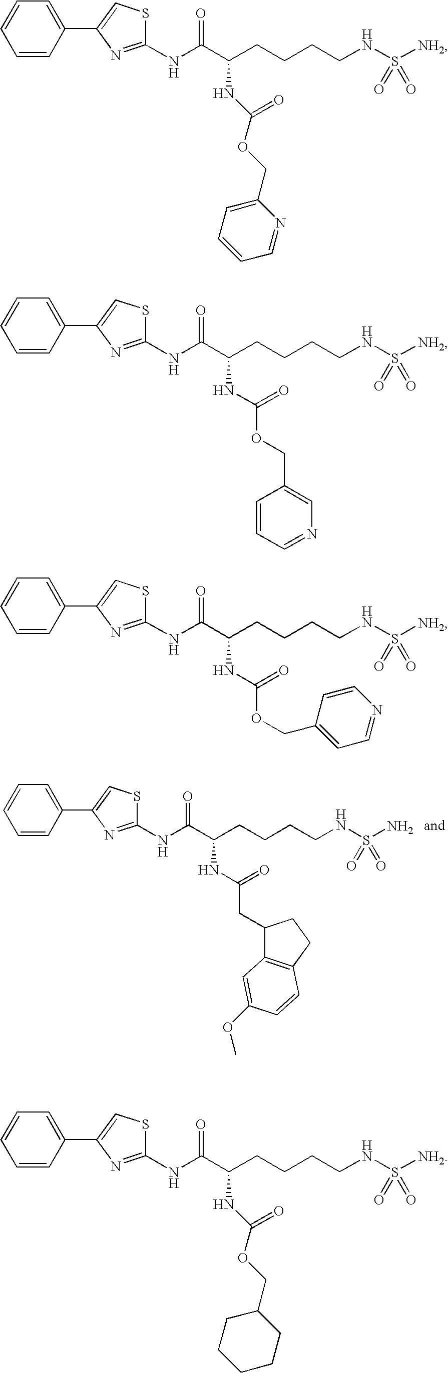

65. The compound according to claim 36, wherein
L is —C₀-C₆alkyl-N(R³)—C(O)—N(R³)—C₀-C₃alkyl-,wherein when the C₀-C₃alkyl is C₁-C₃alkyl, the C₁-C₃ alkyl is optionally substituted with —C(O)—N(R³)—C₀-C₃alkyl-heteroaryl-aryl, —C(O)—N(R³)—C₀-C₃alkyl-heteroaryl, or —C(O)—N(R³)—C₀-C₃alkyl-aryl, wherein each heteroaryl or aryl moeity is optionally substituted; and
Y is optionally substituted aryl, optionally substituted heterocyclyl or optionally substituted cycloalkyl.

66. The compound according to claim 65, wherein Y is an optionally substituted heteroaryl.

67. The compound according to claim 1, selected from the group consisting of

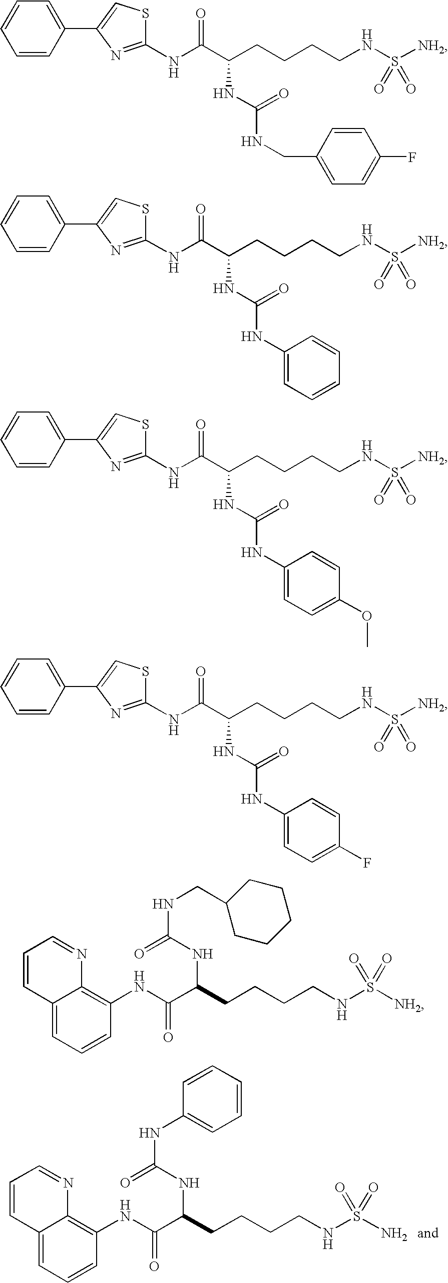

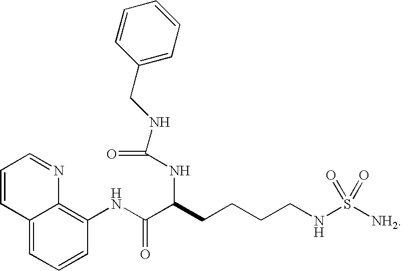

68. The compound according to claim 36, wherein
L is —C₀-C₆alkyl-O—C₀-C₃alkyl-C(O)—N(R³)—C₀-C₃alkyl-, wherein when a C₀-C₃alkyl is C₁-C₃alkyl, the C₁-C₃ alkyl is optionally substituted with —C(O)—N(R³)—C₀-C₃alkyl-heteroaryl, —C(O)—N(R³)—C₀-C₃alkyl-aryl, —C(O)—N(R³)—C₀-C₃alkyl-heteroaryl-aryl, —C(O)—N(R³)—C₀-C₃alkyl-heteroaryl-heteroaryl, —C(O)—N(R³)—C₀-C₃alkyl-aryl-aryl and —C(O)—N(R³)—C₀-C₃alkyl-aryl-heteroaryl, wherein each heteroaryl or aryl moeity is optionally substituted; and
Y is H.

69. The compound according to claim 68, selected from the group consisting of

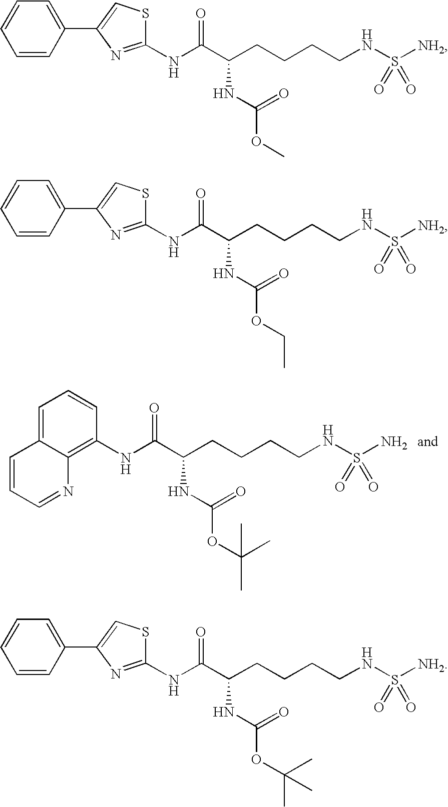

70. The compound according to claim 36, wherein W is further selected from O.

71. The compound according to claim 36 of the formula (IV) 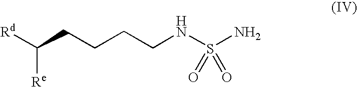
or an N-oxide, hydrate, solvate, pharmaceutically acceptable salt, prodrug or complex thereof, wherein R^{d} and R^{e} are any one of the following combinations:

| R^{d} | R^{e} |
|---|---|
| 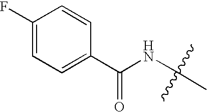 | 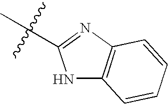 |
| 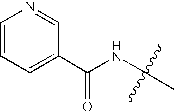 | 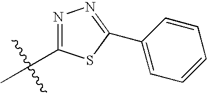 |
| 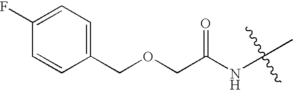 | 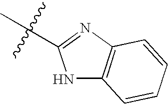 |
| 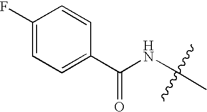 | 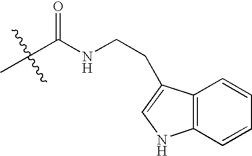 |
| 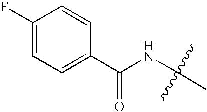 | 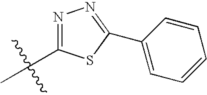 |
| 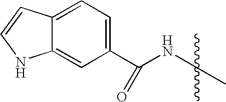 | 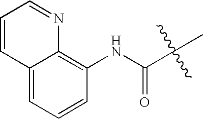 |
| 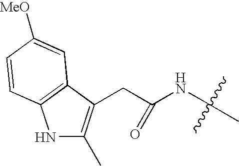 | 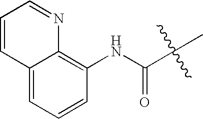 |

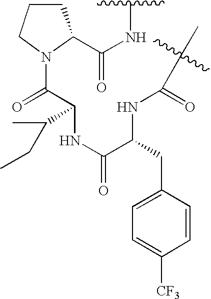

72. The compound according to claim 1, of the formula (V) 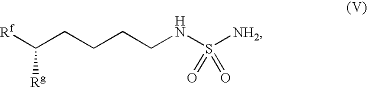
wherein
er an N-oxide, hydrate, solvate, pharmaceutically acceptable salt, prodrug or complex thereof, wherein R^{f} and R^{g} are selected from the group consisting of the following combinations

| R^{g} | R^{f} |
|---|---|
| 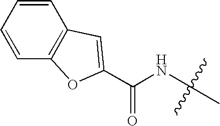 | 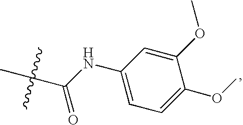 |
| 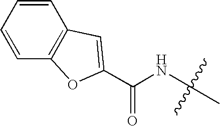 | 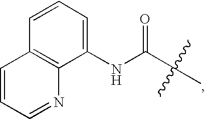 |
| 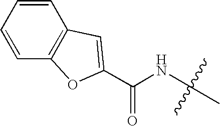 | 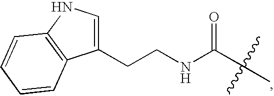 |
| 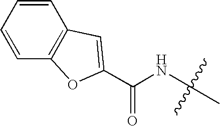 | 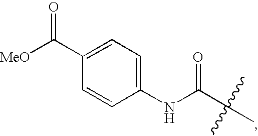 |
| 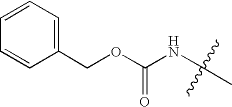 | 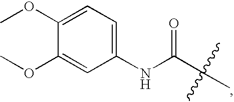 |
| 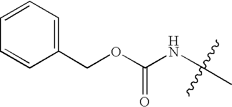 | 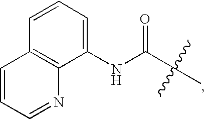 |
| 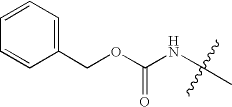 | 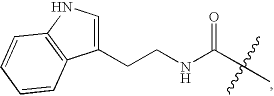 |
| 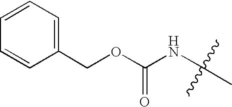 | 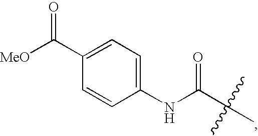 |
| 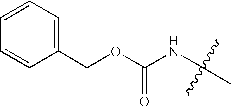 | 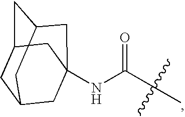 |
| 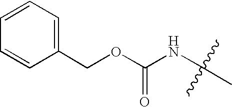 | 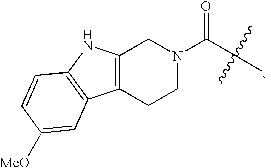 |
| 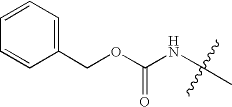 | 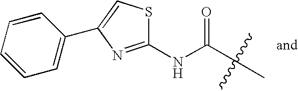 |
| 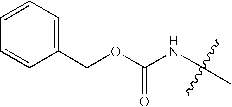 | 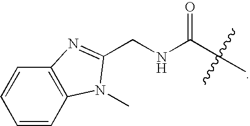 |

73. The compound according to claim 1, wherein 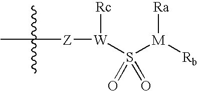
is the structure

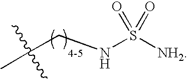

74. The compound according to claim 1, wherein 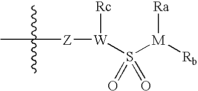
is the structure

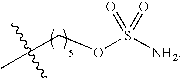

75. The compound according to claim 1, wherein 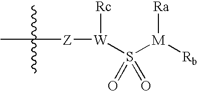
is the structure

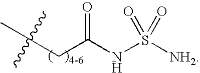

76. A compound according to claim 1 that is selected from the group consisting of

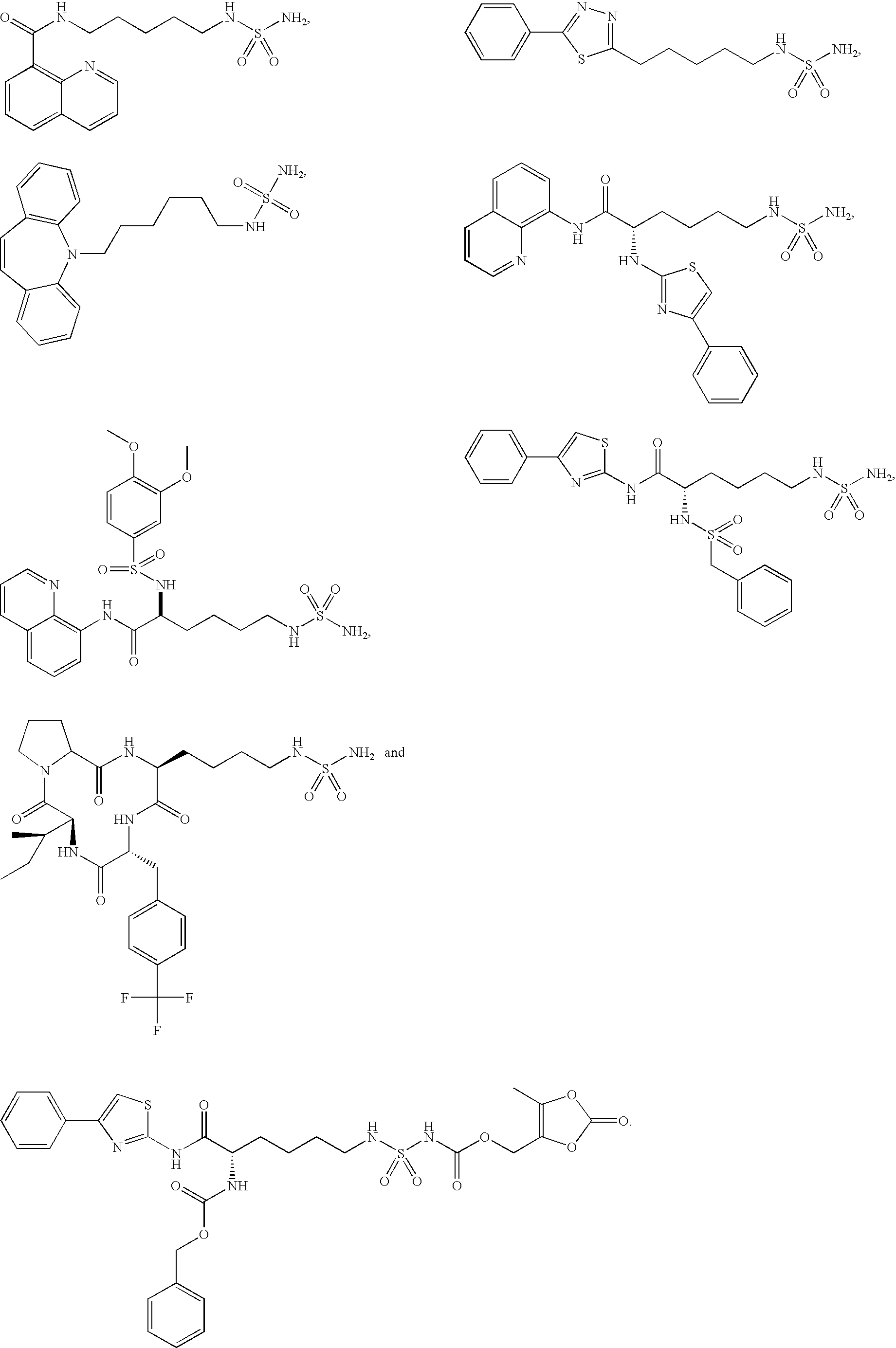

77. A compound according to claim 1 that is selected from the group consisting of

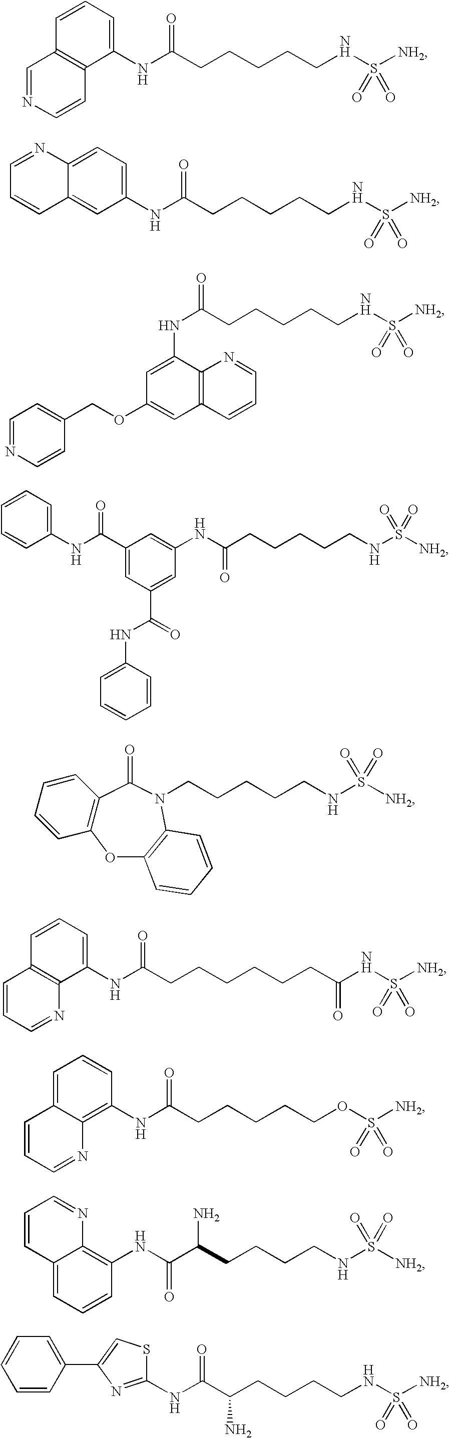

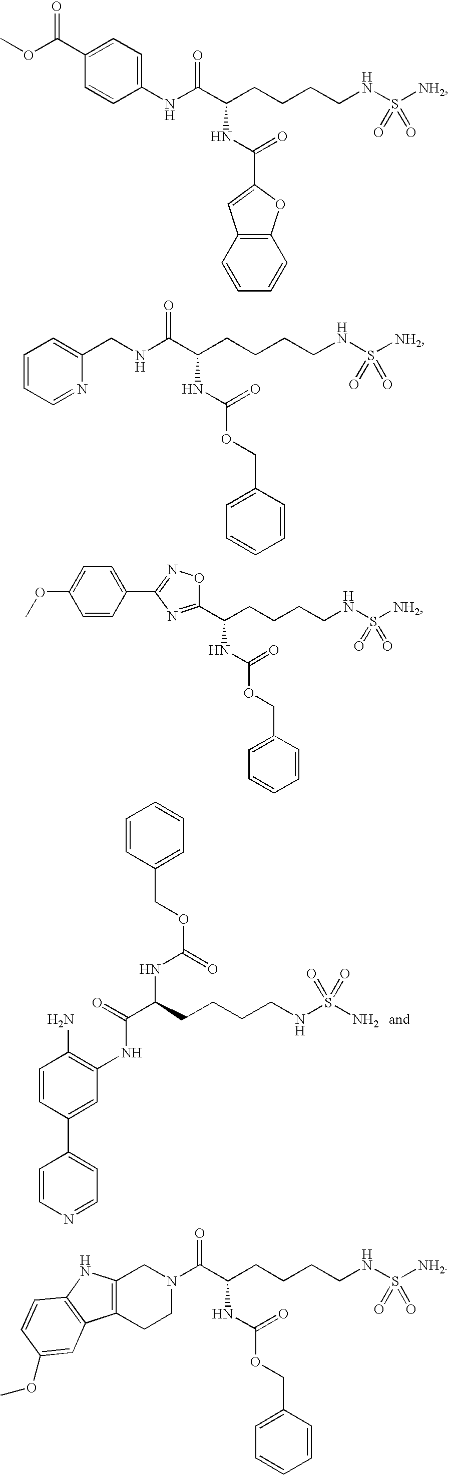

78. A composition comprising a compound according to claim 1 and a pharmaceutically acceptable carrier.

79. The composition according to claim 78, further comprising an additional therapeutic agent.

80. A method of inhibiting histone deacetylase, the method comprising contacting the histone deacetylase with an inhibiting effective amount of a compound according to claim 1 or a composition thereof.

81. A method of inhibiting histone deacetylase in a cell, the method comprising contacting the cell with an inhibiting effective amount of compound according to claim 1 or a composition thereof.
